(19) United States
(12) Patent Application Publication
(10) Pub. No.: US 2025/0098525 A1
(43) Pub. Date: Mar. 20, 2025

(54) COMPOUND AND USE THEREOF IN ORGANIC OPTOELECTRONIC DEVICE

(71) Applicant: SHANGHAI QUADRISTAR ELECTRONIC TECHNOLOGY CO.,LTD., SHANGHAI (CN)

(72) Inventors: PENG WANG, SHANGHAI (CN); XIANGCHENG WANG, SHANGHAI (CN); MU HE, SHANGHAI (CN)

(73) Assignee: SHANGHAI QUADRISTAR ELECTRONIC TECHNOLOGY CO.,LTD., SHANGHAI (CN)

(21) Appl. No.: 18/951,551

(22) Filed: Nov. 18, 2024

Related U.S. Application Data
(63) Continuation of application No. PCT/CN2023/085522, filed on Mar. 31, 2023.

(51) Int. Cl. H10K 85/60; C07C 211/61; C07D 209/88; C07D 209/90; C07D 211/26; C07D 235/06; C07D 241/42; C07D 263/57; C07D 265/38; C07D 307/91; C07D 317/72; C07D 333/76; C07D 337/08; C07D 401/12; C07D 403/04; C07D 403/12; C07D 405/04; C07D 405/10; C07D 405/12; C07D 405/14; C07D 407/12; C07D 409/04; C07D 409/12; C07D 409/14; C07D 413/12; C07D 417/10; C07F 5/02; C07F 7/08; H10K 50/10; H10K 85/40

(52) U.S. Cl. CPC H10K 85/633; C07C 211/61; C07D 209/88; C07D 209/90; C07D 211/26; C07D 235/06; C07D 241/42; C07D 263/57; C07D 265/38; C07D 307/91; C07D 317/72; C07D 333/76; C07D 337/08; C07D 401/12; C07D 403/04; C07D 403/12; C07D 405/04; C07D 405/10; C07D 405/12; C07D 405/14; C07D 407/12; C07D 409/04; C07D 409/12; C07D 409/14; C07D 413/12; C07D 417/10; C07F 5/027; C07F 7/0816; H10K 85/40; H10K 85/636; H10K 85/658; H10K 50/10; H10K 85/615; H10K 85/622; H10K 85/623; H10K 85/624; H10K 85/626; H10K 85/653; H10K 85/654; H10K 85/655; H10K 85/657; H10K 85/6572; H10K 85/6574; H10K 85/6576

(57) ABSTRACT

Embodiments are related to the field of organic electroluminescent materials, and in particular to a compound and a use thereof in an organic optoelectronic device. The chemical structure of the compound is as shown in formula (I). The compound is applied to an organic device, which enables the device to have high hole mobility, and to effectively prevent electrons and excitons from entering a hole transport layer, thereby improving the efficiency of the device. In addition, the molecule has high stability, which further improves the light-emitting efficiency and the service life of the device.

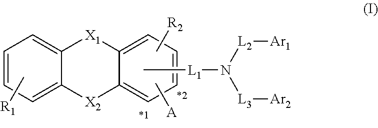

### CROSS-REFERENCE OF RELATED APPLICATION

This application is a continuation of International Application PCT/CN2023/085522 filed on Mar. 31, 2023, which claims priority to Chinese Patent Application No. 202210557908.7, filed on May 19, 2022. The entire disclosures of these applications are incorporated herein by reference.

### TECHNICAL FIELD

The present disclosure relates to the field of organic light emitting diode materials, and in particular to a compound and use thereof in organic optoelectronic device.

### BACKGROUND

Organic light emitting diode (OLED) devices are a type of devices with a sandwich-like structure, which includes a positive electrode film and a negative electrode film as well as an organic functional material layer sandwiched between the two electrode films. At present, this technology has been widely used in display panels of new lighting fixtures, smart phones, tablets and other products, and it will also be further expanded to large-size display products such as televisions. It is a new display technology with fast development and high technical requirements. Common functional organic materials used in OLED devices include hole injection materials, hole transport materials, hole blocking materials, electron injection materials, electron transport materials, electron blocking materials, primary luminescent materials, auxiliary luminescent materials (dyes) and the like. Based on this, the OLED material filed has been committed to developing a new organic OLED material to achieve a low device startup voltage, high luminous efficiency and a better service life. So far, the development of existing OLED optoelectronic functional materials is far behind the requirements of panel manufacturers for OLED materials. Therefore, it is particularly urgent to develop organic functional materials with better performance to meet the current needs of industrial development. Currently, the hole transport material mainly uses aromatic amine compounds with good hole transport properties. N,N′-diphenyl-N,N′-(1-naphthyl)-1,1′-biphenyl-4,4′-diamine (NPB) is widely used in OLED devices with various colors of light, due to its moderate highest occupied orbital energy level and good hole mobility. However, the glass transition temperature of this molecule is low (98° C.), and the device is prone to phase change due to the accumulated Joule heat when working for a long time, which has a great impact on the service life of the device. Therefore, it is necessary to design a hole transport material with both high mobility and glass transition temperature. In addition, in green light devices, there have always been some problems with the lifespan of the hole transport material, which has restricted the use of the devices. Therefore, it is of great significance to develop a hole transport material with high efficiency and long service life.

In the prior art, in order to improve the efficiency and lifetime of the hole transport material, electron-donating groups are added or the degree of conjugation of molecules is increased. For example, Patent Document 1 and Patent Document 2 record that some alkyl groups are introduced on a side chain away from the nitrogen atom, which can improve the migration efficiency of the material. However, with the introduction of the alkyl groups, the thermal stability and electrical stability of the material are reduced, so that the lifetime of the device cannot be guaranteed. Patent Document 3 records that, benzoalkyl groups are introduced into the triarylamine structure which is based on Spiro[fluorene-9,9′-xanthene], and the mobility of the material is improved, but the effect is not significant. According to the simulation calculation results of the molecule, the HOMO and LUMO energy level distributions of the molecules do not have the contribution of the xanthene group, resulting in insufficient improvement in the mobility of the molecule.

Patent Document 4: By introducing a benzene ring near a nitrogen atom, the lifetime of the material is improved, but the mobility needs to be further improved.

### PATENT DOCUMENTATION

Patent Document 1: CN113773207A
Patent Document 2: KR1020220049676A
Patent Document 3: CN114507222A
Patent Document 4: KR1020170092092

### SUMMARY

As described above, in the prior art, the introduction of an alkyl group on the side chain away from the nitrogen atom can improve the mobility of the material, but it has not yet solved the problem of efficiency and lifetime of the material. In view of the above-mentioned shortcomings of the prior art, embodiments of the present disclosure provide a compound and use thereof in an organic optoelectronic device, so as to alleviate the problems in the prior art.

To achieve the above-mentioned objects and other related objects, in one aspect, the embodiments of the present disclosure provides a compound, and a chemical structure of the compound is shown in formula (I):

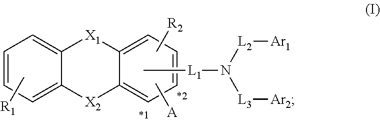

where group A is one or more selected from following groups:

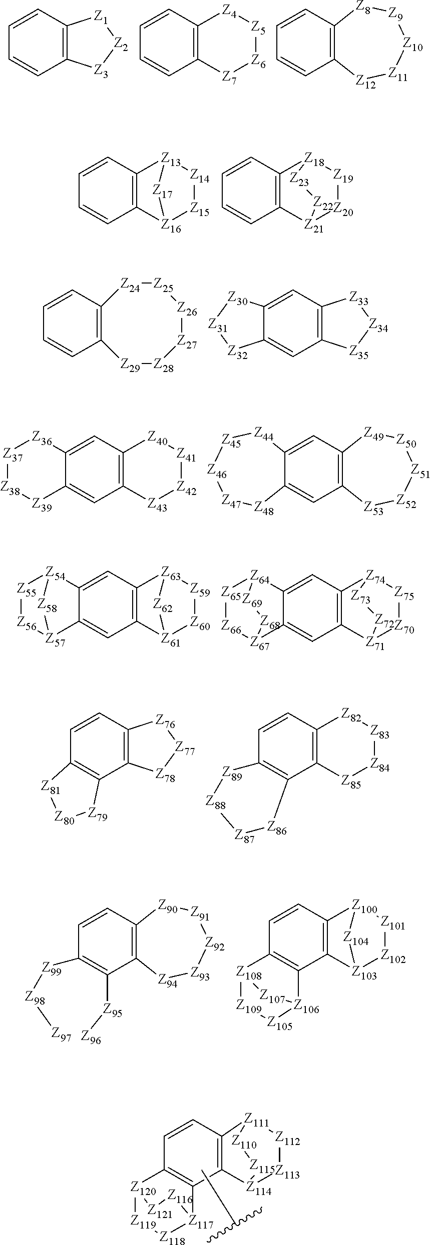

Z₁-Z₇₅, Z₇₆-Z₁₂₁ are each independently selected from CR₃R₄, NR₅, SiR₆R₇, BR₈, O or S. *1 and *2 are connection sites of group A, and *1 or *2 is capable of being connected to any position on group A.

X₁, X₂ are each independently selected from a single bond, CR₉R₁₀, NR₁₁, SiR₁₂R₁₃, O or S. R₁-R₁₃ are the same or different, and are each independently selected from hydrogen, deuterium, substituted or unsubstituted straight or branched C1-C30 alkyl groups, substituted or unsubstituted C1-C30 heteroalkyl groups, substituted or unsubstituted C3-C30 cycloalkyl groups, substituted or unsubstituted C3-C30 heterocycloalkyl groups, substituted or unsubstituted C6-C30 aryl groups, or substituted or unsubstituted C6-C30 heteroaryl groups.

L₁-L₃ are the same or different, and are each independently selected from a single bond, substituted or unsubstituted C6-C30 arylene groups, or substituted or unsubstituted C3-C30 heteroarylene groups.

Ar₁ and Ar₂ are the same or different, and are each independently selected from substituted or unsubstituted C6-C30 aryl groups, or substituted or unsubstituted C6-C30 heteroaryl groups.

In another aspect, the embodiments of the present disclosure provide an organic layer, including the compound as described in the first aspect of the present disclosure.

The compound of the present disclosure can form an organic layer with other components, which can be used in organic optoelectronic devices.

In yet another aspect, the embodiments of the present disclosure provide an organic optoelectronic device, which includes a first electrode, a second electrode and the organic layer as described above in the present disclosure. The organic layer is at least one of a hole injection layer, a hole transport layer, a light-emitting layer, an electron injection layer or an electron transport layer.

In still another aspect, the embodiments of the present disclosure provide a display or lighting device, which includes the organic optoelectronic device as described above in the present disclosure.

Compared with the prior art, the present disclosure has the following beneficial effects:

The compound provided by the embodiments of the disclosure has a benzoalkyl group introduced into the group adjacent to the nitrogen atom. Compared with the aryl group, the benzoalkyl group has better electron transport ability, so that the overall compound has good hole transport performance. In addition, because the introduction of the benzoalkyl group near the nitrogen atom, the triplet energy level of the molecule can be improved, and the triplet state of the material is stabilized, thereby increasing the lifetime. Furthermore, there is a weak conjugation effect between the nitrogen atom and the benzoalkyl group, which enhances the hole transport ability of the molecule. Therefore, when the compounds of the present disclosure are applied to organic devices, it can enable the devices to have high hole mobility, and electrons and excitons can be effectively blocked from entering the hole transport layer, thereby improving the efficiency of the devices. Also, the molecule has high stability, which can further improve the luminous efficiency and service life of the device.

Furthermore, when L₁ is not a single bond, because of the introduction of aromatic groups, the HOMO and LUMO energy levels of the molecule are adjusted to better match the device. In addition, the introduction of aromatic groups lowers the triplet energy level of the molecule and improves the thermal stability of the molecule. What is even more surprising is that, compared with a case where L₁ is a single bond, when L₁ is not a single bond, the efficiency and lifetime of the material molecule are significantly improved, and the material is more suitable for use in blue light devices.

### DETAILED DESCRIPTION OF PREFERRED EMBODIMENTS

Hereinafter, the embodiments of the compound and use thereof in organic optoelectronic devices are described in detail. Those skilled in the art can easily understand other advantages and effects of the present disclosure from the contents disclosed in this specification. The present disclosure may also be implemented or applied through other different specific implementations, and the details in this specification may also be modified or changed in various ways based on different viewpoints and applications without departing from the spirit of the present disclosure.

Before further describing the specific embodiments of the present disclosure, it should be understood that the scope of protection of the present disclosure is not limited to the specific embodiments described below. It should also be understood that the terms used in the examples of the present disclosure are for describing the specific embodiments rather than limiting the scope of protection of the present disclosure. In the present specification and claims, unless otherwise expressly stated herein, the singular forms “a”, “an” and “this” include plural forms.

When numerical ranges are given in the embodiments, it should be understood that the two endpoints of each numerical range and any numerical value between the two endpoints can be used unless otherwise specified in the present disclosure. Unless defined otherwise, all technical and scientific terms used herein have the same meanings as commonly understood by one of ordinary skill in the art. In addition to the specific methods, equipment, and materials used in the embodiments, based on the understanding of the prior art by those skilled in the art and the description of the present disclosure, any methods, equipment, and materials of the prior art that are similar or equivalent to the methods, equipment, and materials described in the embodiments of the present disclosure may also be used to implement the present disclosure.

After extensive research, the inventors of the present disclosure provide a compound based on a series of benzoalkyl. The inventors of the present disclosure found that, by introducing benzoalkane derivatives into a triarylamine system, a series of hole transport materials with excellent performance were obtained. The introduction of the benzoalkane derivatives near the nitrogen atom was originally intended to improve the mobility of the material by utilizing the electron-donating properties of the aliphatic ring. Generally, the thermal stability and service life of the material would decrease after the introduction of the aliphatic ring. However, what is unexpected is that, the lifetime of this material is greatly improved. There are two possible reasons: 1) the introduction of the benzoalkyl group leads to an increase in the steric hindrance between molecular groups, thereby increasing the triplet energy level of the molecules, increasing the triplet stability of the molecules and further increasing the lifetime of the material; and 2) there is a weak conjugation between the benzoalkyl group and the nitrogen atom, which can stabilize the molecule and thus increase the lifetime of the material. Therefore, this type of compound can not also improve the mobility, but also provide a long service life for the OLED device. On this basis, the present disclosure is proposed.

Examples of substituents in the present disclosure are described below, but the substituents are not limited thereto.

[Substituted or unsubstituted] means being substituted with one or more substituents selected from the group consisting of deuterium, a halogen group, a nitrile group, a nitro group, a hydroxyl group, a carbonyl group, an ester group, an imide group, an amino group, a phosphine oxide group, an alkoxy group, an aryloxy group, an alkylthio group, an arylthio group, an alkylsulfonyl group, an arylsulfonyl group, a silyl group, a boron group, an alkyl group, a cycloalkyl group, an alkenyl group, an aryl group, an aralkyl group, an aralkenyl group, an alkylaryl group, an alkylamine group, an aralkylamine group, a heteroarylamine group, an arylamine group, an arylphosphino group and a heteroaryl group, an acenaphthenyl group, a compound group; or being unsubstituted; alternatively, being substituted with a substituent connecting two or more of the substituents exemplified above, or being unsubstituted. For example, “a substituent connecting two or more substituents” may include a biphenyl group, that is, the biphenyl group may be an aryl group, or a substituent connecting two phenyl groups.

The [alkyl] group may be straight or branched, and the number of carbon atoms therein is not particularly limited. In some embodiments, the alkyl includes, but is not limited to, methyl, ethyl, propyl, n-propyl, isopropyl, butyl, n-butyl, isobutyl, tert-butyl, sec-butyl, 1-methyl-butyl, 1-ethyl-butyl, pentyl, n-pentyl, isopentyl, neopentyl, tert-pentyl, hexyl, n-hexyl, 1-methylpentyl, 2-methylpentyl, 4-methyl-2-pentyl, 3,3-dimethylbutyl, 2-ethylbutyl, heptyl, n-heptyl, 1-methylhexyl, cyclopentylmethyl, cyclohexylmethyl, octyl, n-octyl, tert-octyl, 1-methylheptyl, 2-ethylhexyl, 2-propylpentyl, n-nonyl, 2,2-dimethylheptyl, 1-ethyl-propyl, 1,1-dimethyl-propyl, isohexyl, 4-methylhexyl, and 5-methylhexyl.

The above description of alkyl also applies to alkyl in the aralkyl group, aralkylamine group, alkylaryl group and alkylamine group.

The [heteroalkyl group] may be a straight-chain or branched-chain alkyl group containing a heteroatom, and the number of carbon atoms therein is not particularly limited. In some embodiments, the heteroalkyl includes, but is not limited to, alkoxy, alkylthio, alkylsulfonyl, and the like. The alkoxy includes, but is not limited to, methoxy, ethoxy, n-propoxy, isopropoxy, i-propyloxy, n-butoxy, isobutoxy, tert-butoxy, sec-butoxy, n-pentoxy, neopentoxy, isopentoxy, n-hexyloxy, 3,3-dimethylbutoxy, 2-ethylbutoxy, n-octoxy, n-nonyloxy, n-decyloxy, benzyloxy, p-methylbenzyloxy, and the like. The alkylthio for example includes, but is not limited to, methylthio, ethylthio, n-propylthio, isopropylthio, isopropylthio, n-butylthio, isobutylthio, tert-butylthio, sec-butylthio, n-pentylthio, neopentylthio, isopentylthio, n-hexylthio, 3,3-dimethylbutylthio, 2-ethylbutylthio, n-octylthio, n-nonylthio, n-decylthio, benzylthio, and the like.

The [cycloalkyl group] may be cyclic, and the number of carbon atoms therein is not particularly limited. In some embodiments, the cycloalkyl includes, but is not limited to, cyclopropyl, cyclobutyl, cyclopentyl, 3-methylcyclopentyl, 2,3-dimethylcyclopentyl, cyclohexyl, 3-methylcyclohexyl, 4-methylcyclohexyl, 2,3-dimethylcyclohexyl, 3,4,5-trimethylcyclohexyl, 4-tert-butylcyclohexyl, cycloheptyl, cyclooctyl, and the like.

The [heterocycloalkyl group] may be a cycloalkyl group containing a heteroatom, and the number of carbon atoms therein is not particularly limited. In some embodiments, the heterocycloalkyl includes, but is not limited to,

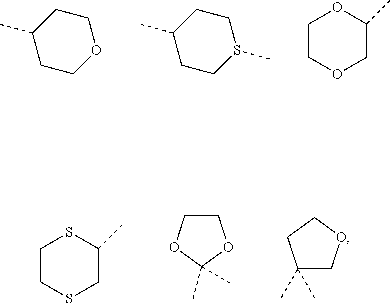

and the like.

For [aryl group], there are no particular limitations on the aryl group, and the aryl group may be a monocyclic aryl group or a polycyclic aryl group. In some embodiments, the monocyclic aryl group includes, but is not limited to, phenyl, biphenyl, terphenyl, quaterphenyl, pentphenyl, and the like. The polycyclic aromatic group includes, but is not limited to, naphthyl, anthracenyl, phenanthrenyl, pyrenyl, perylenyl, fluorenyl, and the like. The fluorenyl group may be substituted, for example, 9,9′-dimethylfluorenyl, 9,9′-dibenzofluorenyl, and the like. In addition, two of the substituents may be combined with each other to form a spiro ring structure, such as 9,9′-spirobifluorenyl and the like.

The above description of the aryl group can be applied to the arylene group, except that the arylene group is divalent.

The above description of the aryl group may be applied to aryl in the aryloxy group, arylthio group, arylsulfonyl group, arylphosphino group, arylalkyl group, arylalkylamino group, arylalkenyl group, alkylaryl group, arylamine group and arylheteroarylamine group.

The [heteroaryl group] contains one or more of N, O, P, S, Si and Se as heteroatoms. The heteroaryl includes, but is not limited to, pyridinyl, pyrrolyl, pyrimidinyl, pyridazinyl, furanyl, thienyl, imidazolyl, pyrazolyl, oxazolyl, isoxazolyl, thiazolyl, isothiazolyl, triazolyl, diazolyl, thiadiazolyl, dithiazolyl, tetrazolyl, pyranyl, thiopyranyl, pyrazinyl, azinyl, thiazinyl, dioxinyl, triazinyl, tetrazinyl, quinolyl, isoquinolyl, quinolyl, quinazolinyl, quinoxalinyl, naphthyridinyl, acridinyl, xanthenyl, phenanthridinyl, phthalazine, triazatruxene, indolyl, indolinyl, indolizinyl, phthalazinyl, pyridopyrimidinyl, pyridopyrazinyl, pyrazinopyrazinyl, benzothiazolyl, benzoxazolyl, benzimidazolyl, benzothienyl, benzofuranyl, dibenzothienyl, dibenzofuranyl, carbazolyl, benzocarbazolyl, dibenzocarbazolyl, indolecarbazolyl, indenocarbazolyl, phenazinyl, imidazopyridinyl, phenazine, phenanthridinyl, phenanthroline, phenothiazinyl, imidazopyridinyl, imidazophenanthridinyl, benzimidazoquinazolinyl, benzimidazophenanthridinyl, spiro[fluorene-9,9′-xanthene], benzophenaphthyl, dinaphthofuranyl, naphthiobenzofuranyl, dinaphthothiophene, naphthiobenzothiophene, triphenylphosphine oxide, triphenylborane, etc.

The above description of the heteroaryl group may be applied to the heteroaryl in the heteroarylamine group and the arylheteroarylamine group.

The above description of the heteroaryl group may be applied to heteroarylene group, except that the heteroarylene group is divalent.

In one aspect, the embodiments of the present disclosure provides a compound, and the chemical structure of the compound is shown in formula (I):

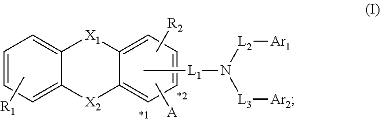

where group A is one or more selected from the following groups:

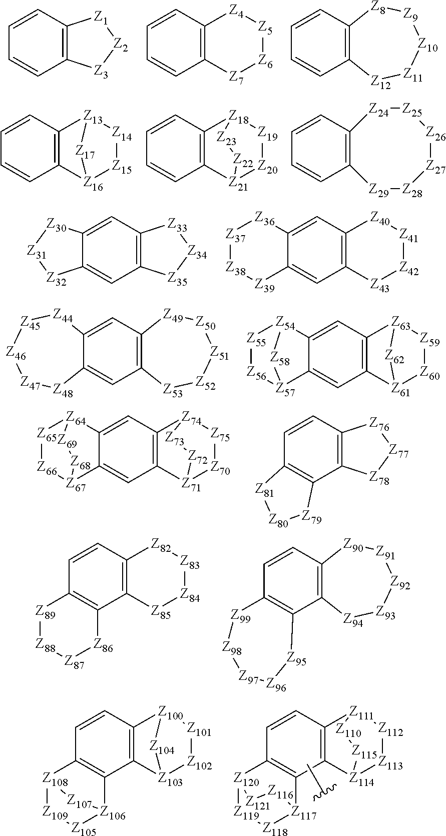

Z₁-Z₇₅, Z₇₆-Z₁₂₁ are each independently selected from CR₃R₄, NR₅, SiR₆R₇, BR₈, O or S. *1 and *2 are connection sites of group A, and *1 or *2 is capable of being connected to any position on group A.

X₁, X₂ are each independently selected from a single bond, CR₉R₁₀, NR₁₁, SiR₁₂R₁₃, O or S, or R₉, R₁₀ are bonded to form a ring. A single bond is a type of direct bond. For example, X₁ is a single bond, representing a direct connection to the carbons on two benzene rings connected to X₁. With regard to the ring bonded by R₉ and R₁₀, it means forming an aliphatic hydrocarbon ring, an aromatic hydrocarbon ring, an aliphatic heterocyclic ring, an aromatic heterocyclic ring, or a condensed ring thereof. For example, R₉ and R₁₀ are bonded to form

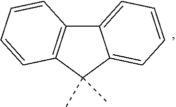

which is combined into formula (I) to form

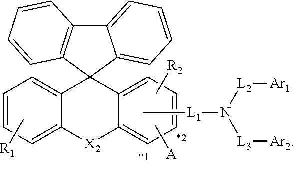

R₁-R₁₃ are the same or different, and are each independently selected from hydrogen, deuterium, substituted or unsubstituted straight or branched C1-C30 alkyl groups, substituted or unsubstituted C1-C30 heteroalkyl groups, substituted or unsubstituted C3-C30 cycloalkyl groups, substituted or unsubstituted C3-C30 heterocycloalkyl groups, substituted or unsubstituted C6-C30 aryl groups, or substituted or unsubstituted C6-C30 heteroaryl groups.

Among them, R₁ and R₂ not only represent a single substituent group, but also represent multiple identical or different substituent groups. For example, it may be selected from the following structures:

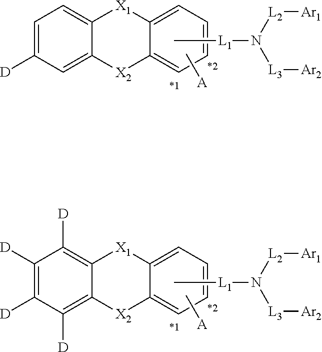

L₁-L₃ are the same or different, and each are independently selected from a single bond, substituted or unsubstituted C6-C30 arylene groups, or substituted or unsubstituted C3-C30 heteroarylene groups.

Ar₁ and Ar₂ are the same or different, and are each independently selected from substituted or unsubstituted C6-C30 aryl groups, or substituted or unsubstituted C6-C30 heteroaryl groups.

When Z (Z₁-Z₇₅, Z₇₆-Z₁₂₁) mentioned above is selected from CR₃R₄, NR₅ or SiR₆R₇, and when X (X₁, X₂) is selected from CR₉R₁₀, NR₁₁, or SiR₁₂R₁₃, since the fatty alkyl group, the nitrogen atom and the silane group have an electron donating effect, the A ring has an electron donating effect, which can fully stabilize the nitrogen atom of the triarylamine, and make it more stable, thereby improving the lifetime of the material. When Z (Z₁-Z₇₅, Z₇₆-Z₁₂₁) is selected from BRs, O or S, and when X (X₁, X₂) is selected from O or S, due to the electron-withdrawing properties of BRs, O or S, electrons would shift toward them, thus affecting the lifetime of the material. But surprisingly, in the case where Z (Z₁-Z₇₅, Z₇₆-Z₁₂₁) is selected from BRs, O or S, when the material is applied to red light devices, there is basically no effect on the lifetime; however, when the material is applied to green light devices, their lifetime is lower than that when Z is selected from groups having the electron-donating effect. The possible reason is that, in the green light devices, the energy of excitons is high and thus there is high stability requirements on the molecules; as thus, there is no difference in lifetime when being used in the red light devices, but has differences in efficiency and lifetime when being used in the green light devices.

In some embodiments, in formula (I), the number of carbon atoms in the aforementioned alkyl group may also be 1 to 10, 1 to 20, or 20 to 30, etc. The number of carbon atoms in the aforementioned cycloalkyl group may be 3 to 10, 3 to 20, or 3 to 30. The number of carbon atoms in the aforementioned heteroalkyl group may also be 3 to 10, 1 to 20, or 20 to 30. The number of carbon atoms in the aforementioned heterocycloalkyl group may also be 3 to 10, 3 to 20, or 20 to 30. The number of carbon atoms in the aforementioned aryl group may be 6 to 10, 6 to 20, or 20 to 30. The number of carbon atoms in the aforementioned heteroaryl group may also be 6 to 10, 6 to 20, or 20 to 30.

The above description of the number of carbon atoms in the aryl group and the heteroaryl group is applicable to the arylene group and the heteroarylene group mentioned in the present disclosure.

In the compound provided by the present disclosure, group A is any one selected from following groups:

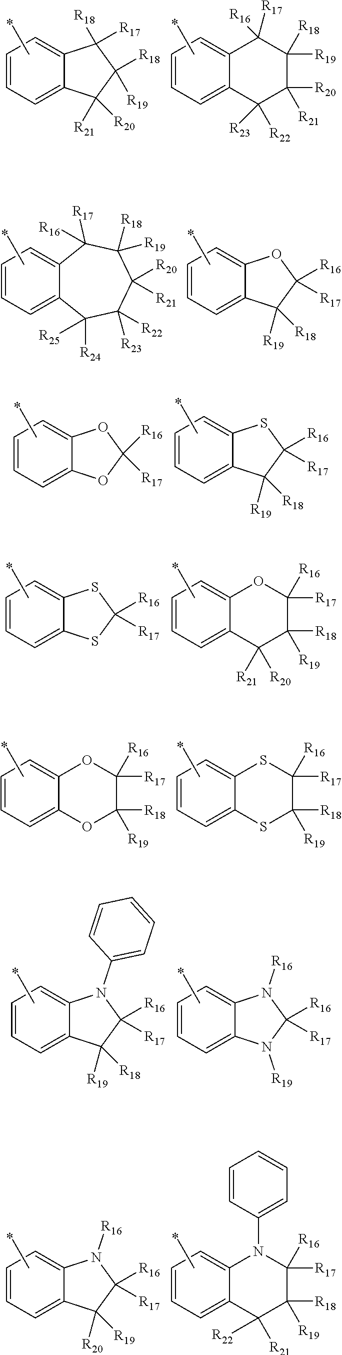

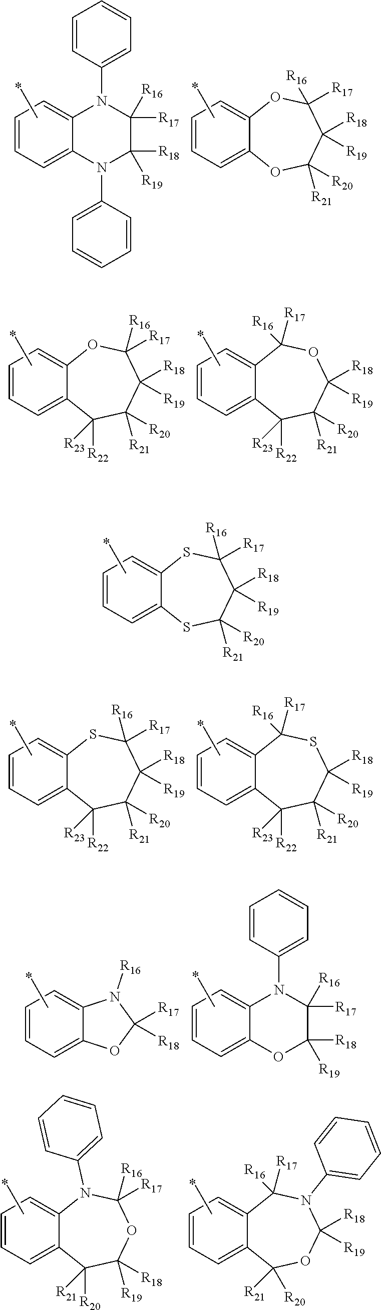

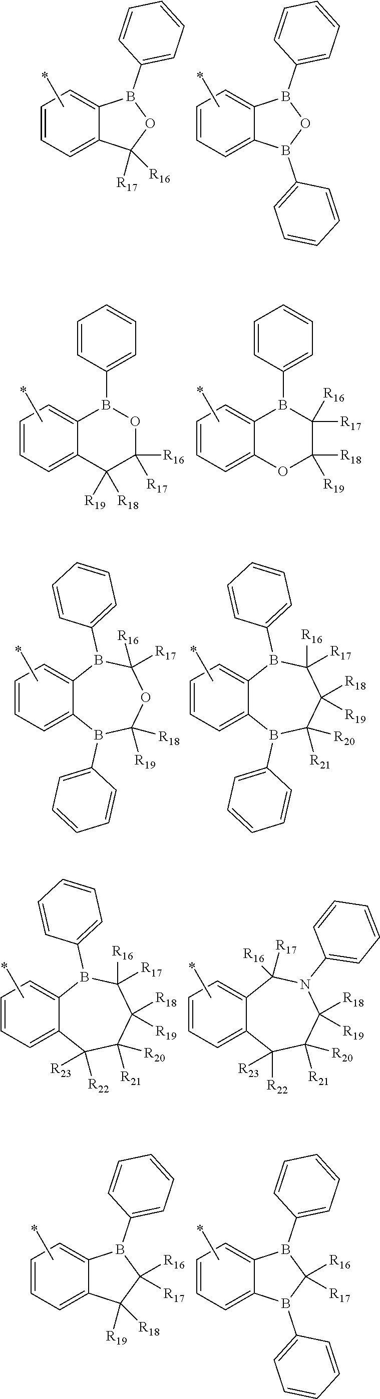

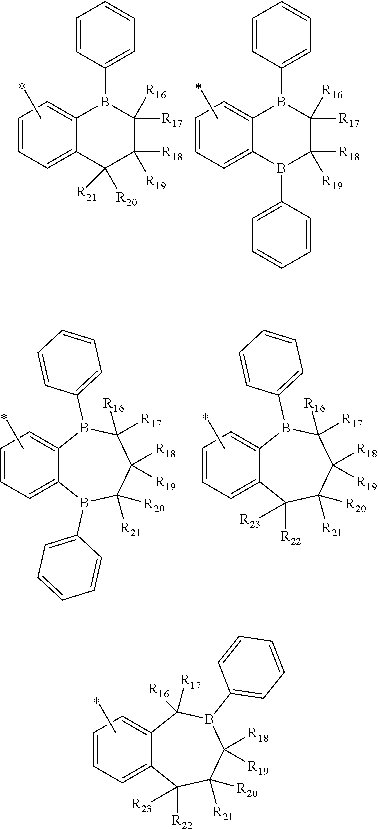

where R₁₆-R₂₃ are each independently selected one or more from hydrogen, deuterium, substituted or unsubstituted C1-C60 alkyl groups, substituted or unsubstituted C1-C60 cycloalkyl groups, substituted or unsubstituted C1-C60 heteroalkyl groups, substituted or unsubstituted C1-C60 heterocycloalkyl groups, substituted or unsubstituted C1-C60 aryl groups or substituted or unsubstituted C1-C60 heteroaryl groups. [symbol]
   is the connection site of the atom, which is not limited to a single link, but may also represent multiple links. The connection site is not limited to the ring of the aforementioned group A, but also represents any position of the groups shown. It may also represent bonding with adjacent atoms to form a ring.

In the compounds provided by the present disclosure, in some embodiments, group A is any one selected from the following groups:

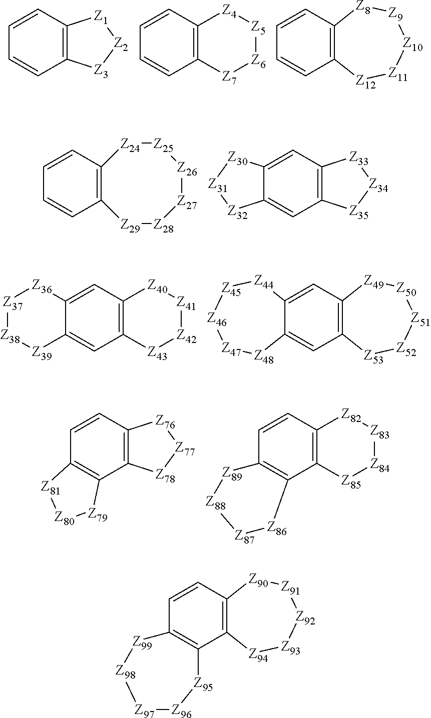

Preferably, it is

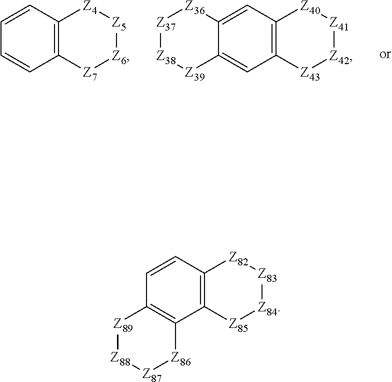

Further, group A is one or more selected from the following groups:

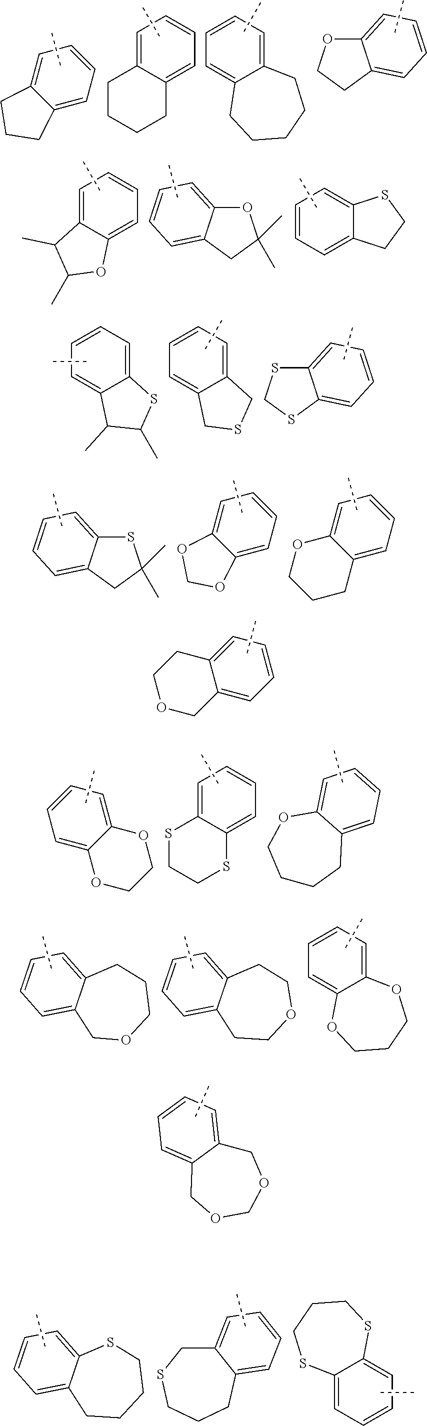

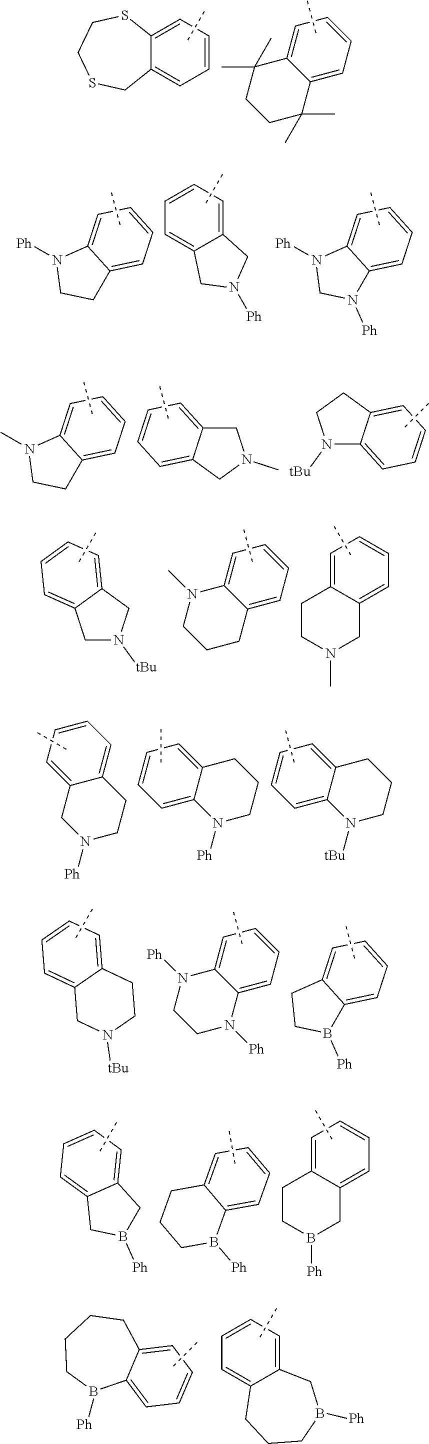

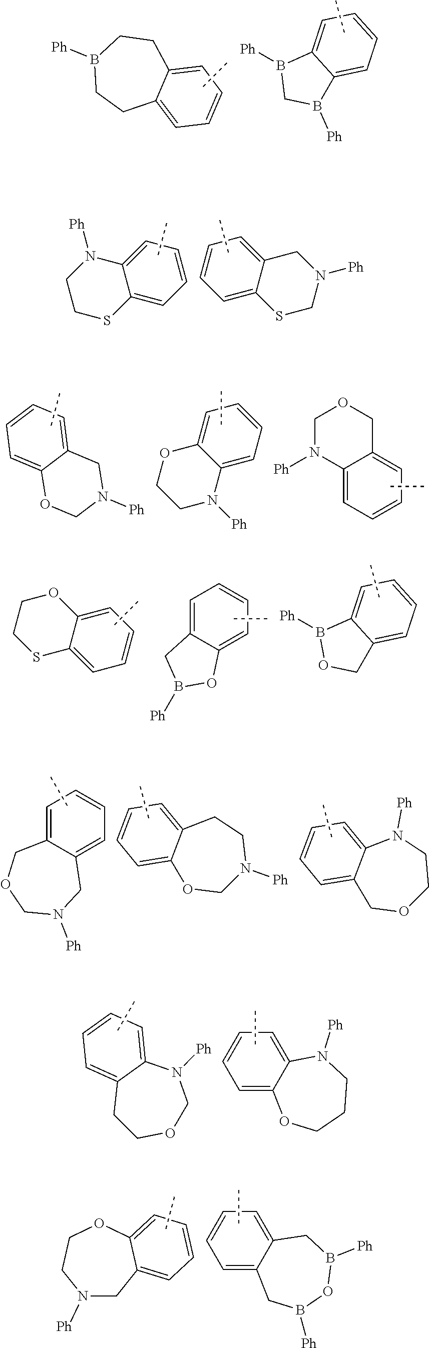

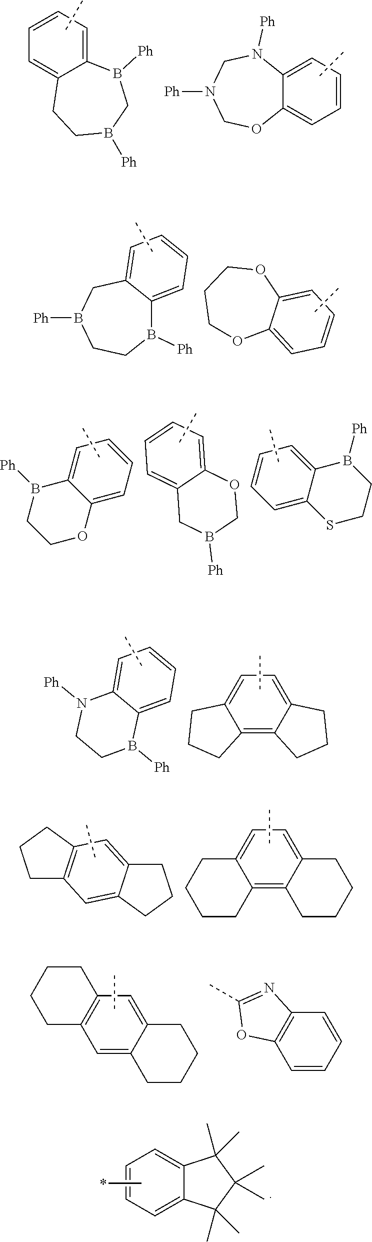

In the compounds provided by the present disclosure, X₁ and X₂ are the same or different, and are each independently selected from a single bond, O, S,

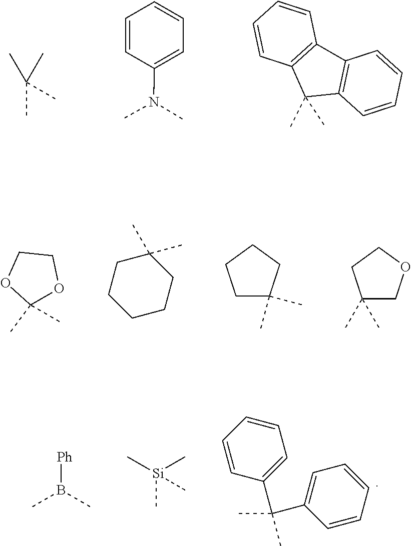

The above ----- represents a connecting bond, connecting to the adjacent group. For example, in X₁, ------ represents connection to a benzene ring. For example,

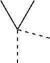

connects to an adjacent benzene ring to form

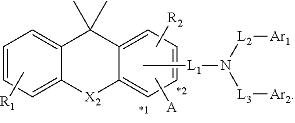

For another example, in X₂, ------ represents connection to a benzene ring.

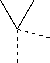

connects to the adjacent benzene ring to form

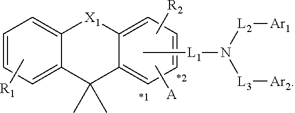

Considering the simplicity and cost of synthesis, R₁ and R₂ are selected from hydrogen.

Generally, the lifetime of the material is improved by replacing hydrogen atoms with deuterium atoms, and therefore R₁ and R₂ are preferably selected from deuterium.

In the compounds provided by the present disclosure, R₃-R₁₃ are the same or different, and are each independently selected from hydrogen, deuterium, substituted or unsubstituted straight or branched C1-C30 alkyl groups, substituted or unsubstituted C1-C12 alkoxy groups, substituted or unsubstituted C1-C12 alkylthio groups, substituted or unsubstituted C3-C30 cycloalkyl groups, substituted or unsubstituted C3-C30 heterocycloalkyl groups, substituted or unsubstituted C6-C30 aryl groups, or substituted or unsubstituted C6-C30 heteroaryl groups.

In the compounds provided by the present disclosure, the L₁-L₃ are each independently selected from a single bond, a substituted or unsubstituted phenylene group, a substituted or unsubstituted phenylene group, a substituted or unsubstituted naphthylene group, a substituted or unsubstituted biphenylene group, a substituted or unsubstituted terphenylene group, a substituted or unsubstituted anthrylene group, a substituted or unsubstituted phenanthrenyl group,

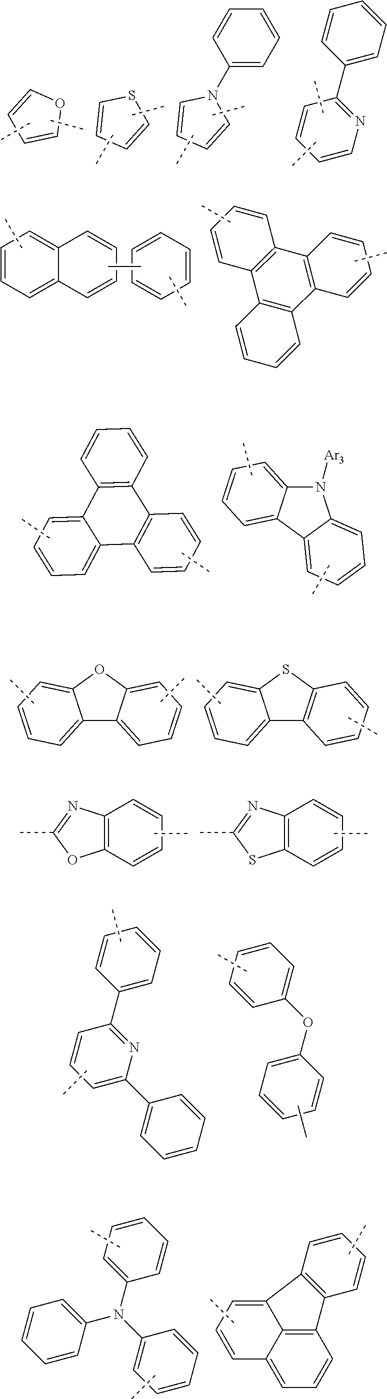

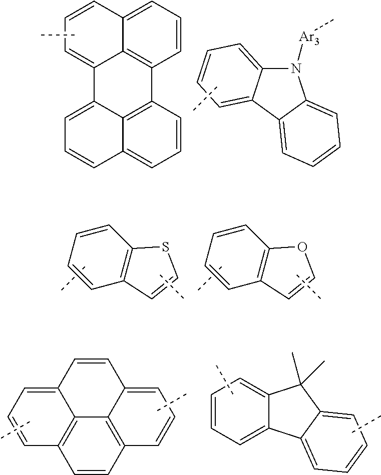

and the like. Ar₃ is selected from C6-C20 aryl groups or C2-C15 heteroaryl groups. Ar₃ is preferably phenyl, naphthyl, etc.

Preferably, L₁-L₃ are each independently selected from phenylene, naphthylene, biphenylene,

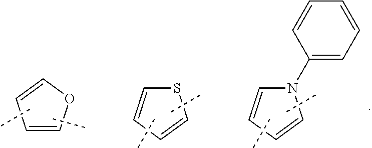

The above ----- represents a connecting bond, connecting to the adjacent group. For example, in L₂, ----- represents connection with Ar₁ or N. For another example, in L₃, ----- represents connection with Ar₂ or N.

In the compounds provided by the present disclosure, Ar₁ and Ar₂ are each independently selected from a substituted or unsubstituted phenyl group, a substituted or unsubstituted naphthyl group, a substituted or unsubstituted biphenyl group, a substituted or unsubstituted terphenyl group,

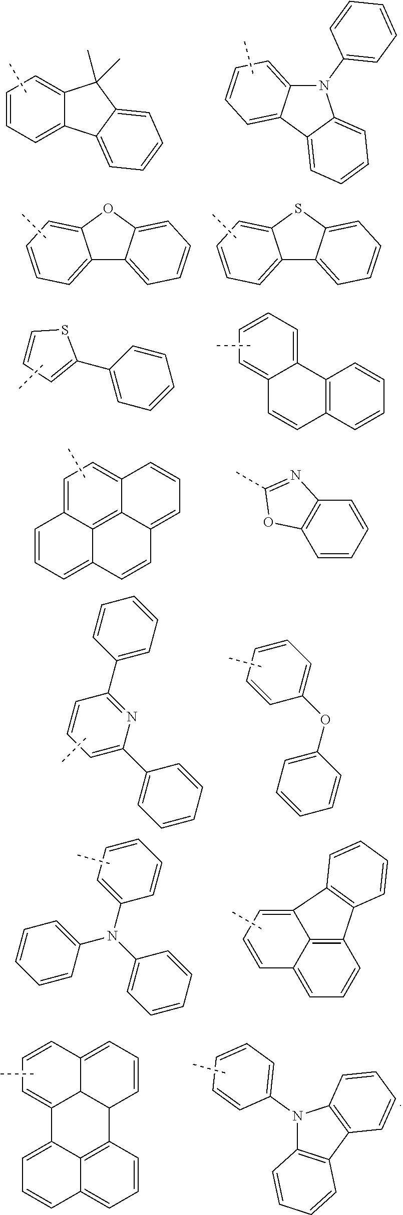

Considering the simplicity and cost of molecular synthesis, one of X₁ and X₂ is a single bond and the other is CR₉R₁₀. This can be inferred unambiguously from the compound of formula (I). That is, X₁ is selected from a single bond, X₂ is selected from CR₉R₁₀, and the following general compound is obtained when other conditions remain unchanged.

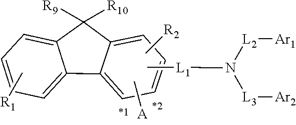

R₁, R₂, A, L₁, L₂, L₃, Ar₁, and Ar₂ are as defined in the compound of formula (I).

R₉ and R₁₀ are independently selected from substituted or unsubstituted straight or branched C1-C30 alkyl groups, substituted or unsubstituted C1-C12 alkoxy groups, substituted or unsubstituted C1-C12 alkylthio groups, substituted or unsubstituted C3-C30 cycloalkyl groups, substituted or unsubstituted C3-C30 heterocycloalkyl groups, substituted or unsubstituted C6-C30 aryl groups, or substituted or unsubstituted C6-C30 heteroaryl groups; or are bonded to adjacent groups to form a ring. From the perspective of simplicity and cost of molecular synthesis, when L₁ is selected from a single bond, one of X₁ and X₂ is a single bond and the other is CR₉R₁₀. This can be inferred unambiguously from the compound of formula (I). That is, when X₁ is selected from a single bond, X₂ is selected from CR₉R₁₀, and other conditions remain unchanged, the compound represented by formula (II) is obtained.

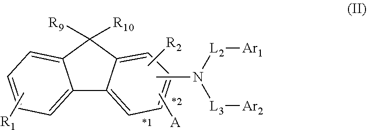

In which, R₁ and R₂ are each independently selected from hydrogen and deuterium.

R₉ and R₁₀ are independently selected from substituted or unsubstituted straight or branched C1-C30 alkyl groups, substituted or unsubstituted C1-C12 alkoxy groups, substituted or unsubstituted C1-C12 alkylthio groups, substituted or unsubstituted C3-C30 cycloalkyl groups, substituted or unsubstituted C3-C30 heterocycloalkyl groups, substituted or unsubstituted C6-C30 aryl groups, or substituted or unsubstituted C6-C30 heteroaryl groups; or are bonded to adjacent groups to form a ring. A, L₂, L₃, Ar₁, and Ar₂ are as defined in the compound of formula (I).

Preferably, X₁ and X₂ described in the specification are each independently selected from a single bond,

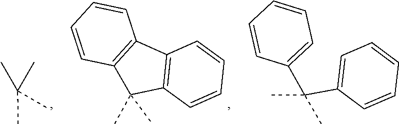

and the like. The chemical formula (II) may be represented by the following structural formula:

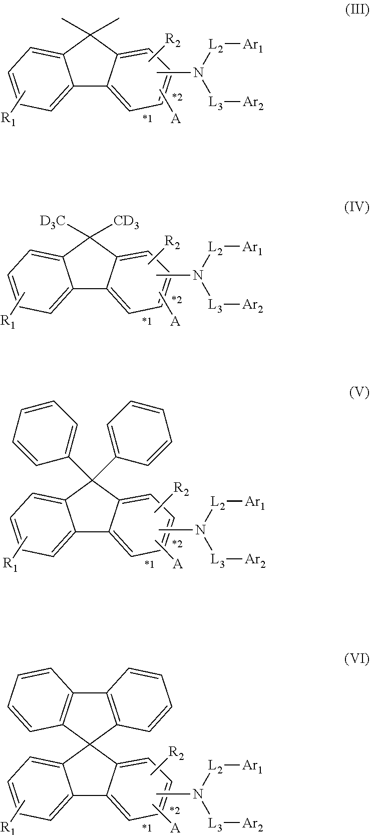

where R₁, R₂, A, L₂, L₃, Ar₁, and Ar₂ are as defined in the compound of formula (II).

Preferably, ring A is selected from the following structures:

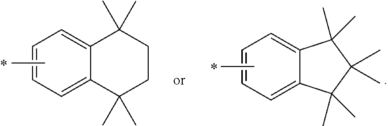

Preferably, the compound has the structural formula

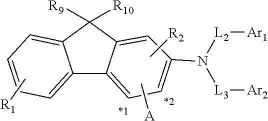

where R₁, R₂, R₉, R₁₀, A, L₂, L₃, Ar₁, and Ar₂ are as defined in the compound of formula (II).

Further preferably, the structural formula of the compound is:

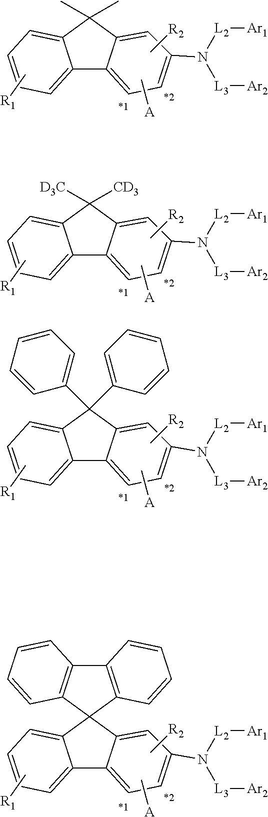

where R₁, R₂, A, L₂, L₃, Ar₁, and Ar₂ are as defined in the compound of formula (II).

From the perspective of simplicity and cost of molecular synthesis, when L₁ is selected from phenyl, one of X₁ and X₂ is a single bond and the other is CR₉R₁₀. This can be inferred unambiguously from the compound of formula (I). That is, when X₁ is selected from a single bond, X₂ is selected from CR₉R₁₀, L₁ is selected from phenyl, and other factors remain unchanged, the compound represented by formula (VII) is obtained.

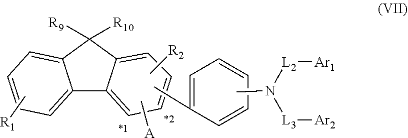

where R₁ and R₂ are each independently selected from hydrogen or deuterium.

In which, R₉ and R₁₀ are independently selected from substituted or unsubstituted straight or branched C1-C30 alkyl groups, substituted or unsubstituted C1-C12 alkoxy groups, substituted or unsubstituted C1-C12 alkylthio groups, substituted or unsubstituted C3-C30 cycloalkyl groups, substituted or unsubstituted C3-C30 heterocycloalkyl groups, substituted or unsubstituted C6-C30 aryl groups, or substituted or unsubstituted C6-C30 heteroaryl groups; or are bonded to adjacent groups to form a ring. A, L₂, L₃, Ar₁, and Ar₂ are as defined in the compound of formula (I).

Preferably, X₁ and X₂ described in the specification are each independently selected from a single bond,

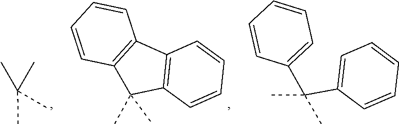

and the like. The chemical formula (VII) may be represented by the following structural formula:

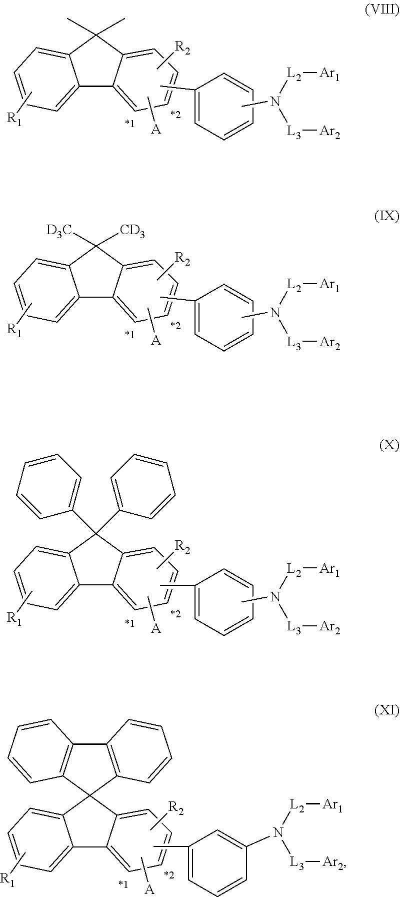

where R₁, R₂, A, L₂, L₃, Ar₁, and Ar₂ are as defined in the compound of formula (VII).

From the perspective of the cost of synthesis and the availability of raw materials, preferably, the A ring is selected from the following structures:

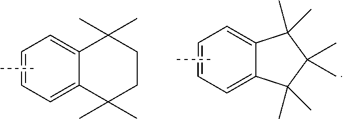

Preferably, the compound has the structural formula

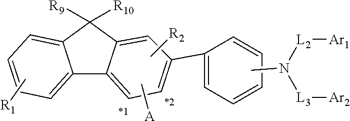

where R₁, R₂, R₉, R₁₀, A, L₂, L₃, Ar₁, and Ar₂ are as defined in the compound of formula (VII).

Further preferably, the structural formula of the compound is:

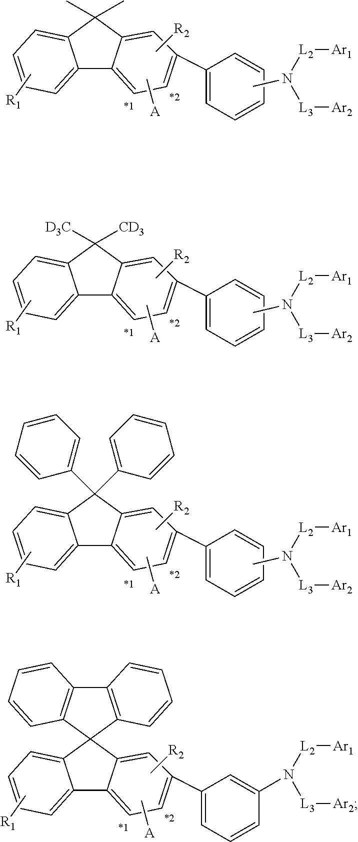

where R₁, R₂, A, L₂, L₃, Ar₁, and Ar₂ are as defined in the compound of formula (VII).

In some embodiments, the chemical structure of the compound is selected from following structures:

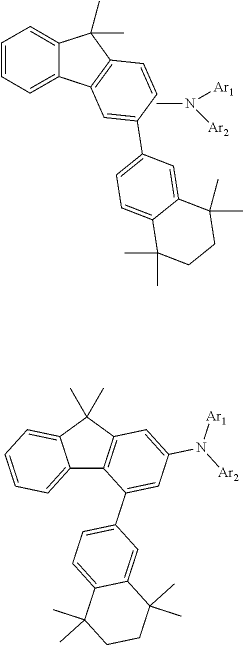

where Ar₁ and Ar₂ are each independently selected from phenyl, diphenyl, terphenyl, naphthyl, phenanthryl, dimethylfluorene, diphenylfluorene, spirofluorene, dibenzothienyl, dibenzofuranyl, carbazolyl, or a combination of any two of these groups. When Ar₁ and Ar₂ are selected from dimethylfluorene, diphenylfluorene, and spirofluorene, their adjacent N position is not connected with other groups. Ar₁ and Ar₂ are not selected as a combination of diphenyl and carbazolyl.

In the compound provided by the present disclosure, the compound is any one selected from the following chemical structures:

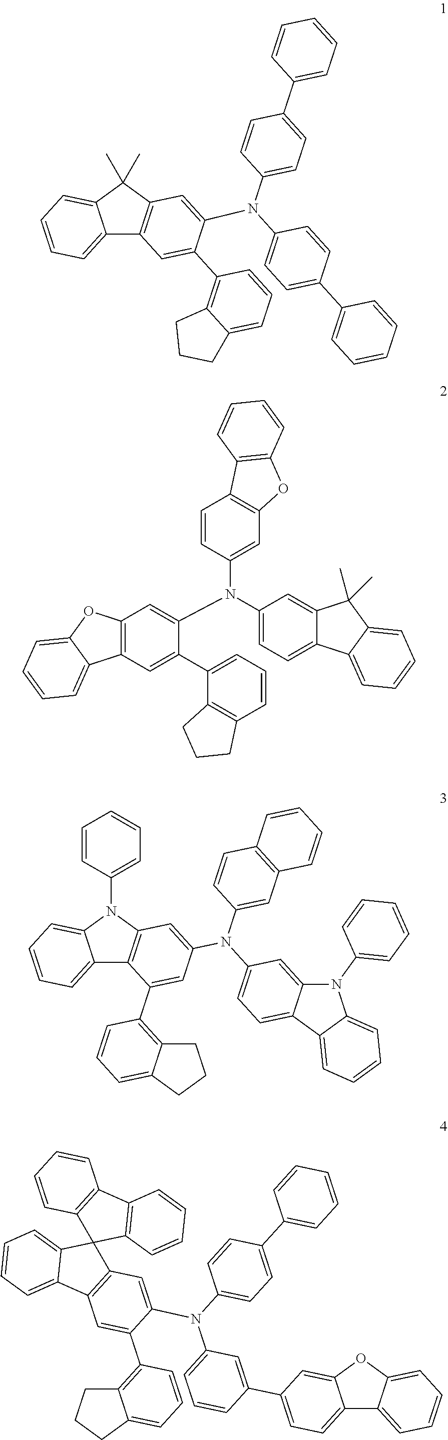

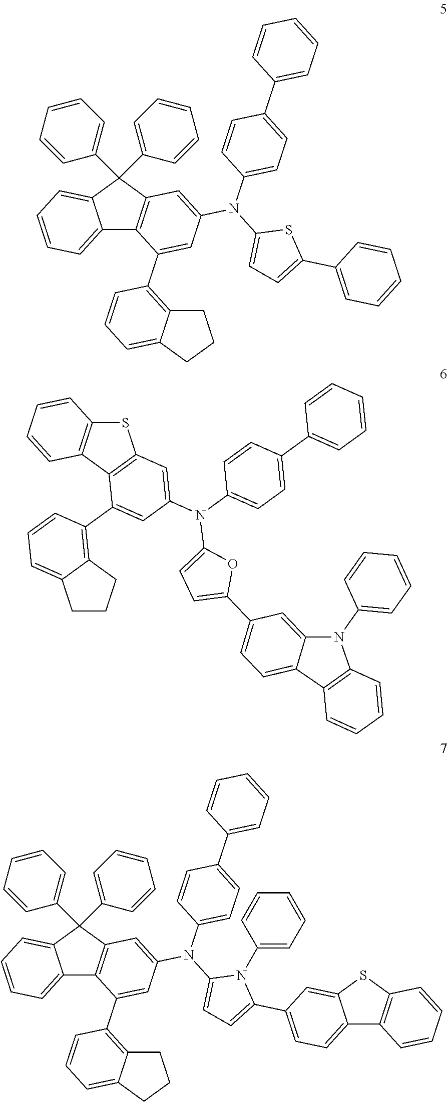 [SMILES: c1ccc(-c2ccc(N(c3cc(-c4cccc5c4CCC5)c4c(c3)C(c3ccccc3)(c3ccccc3)c3ccccc3-4)c3ccc(-c4ccccc4)s3)cc2)cc1 ; c1ccc(-c2ccc(N(c3cc(-c4cccc5c4CCC5)c4c(c3)sc3ccccc34)c3ccc(-c4ccc5c6ccccc6n(-c6ccccc6)c5c4)o3)cc2)cc1 ; c1ccc(-c2ccc(N(c3cc(-c4cccc5c4CCC5)c4c(c3)C(c3ccccc3)(c3ccccc3)c3ccccc3-4)c3ccc(-c4ccc5c(c4)sc4ccccc45)n3-c3ccccc3)cc2)cc1]

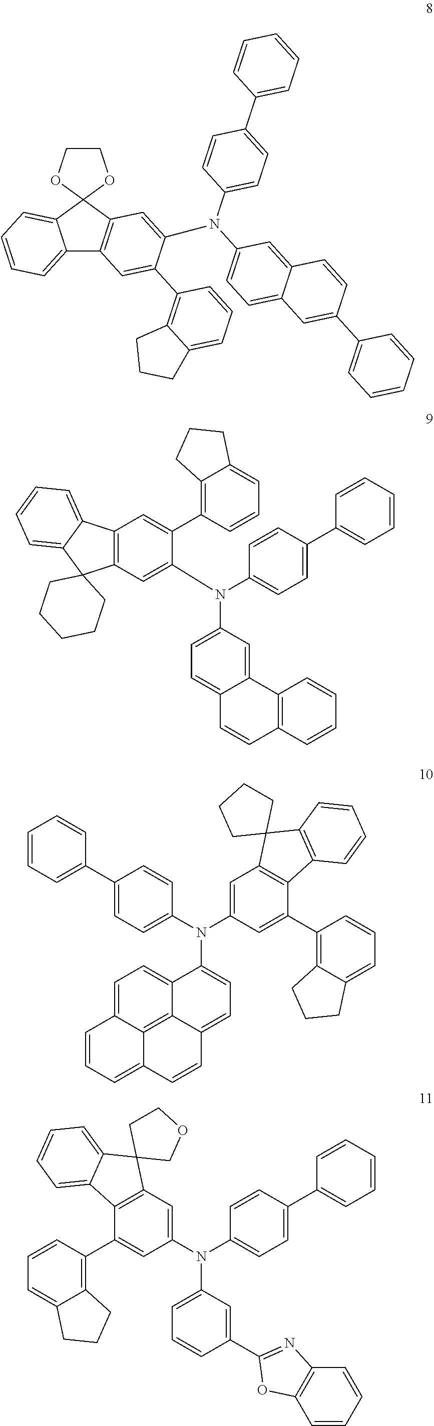

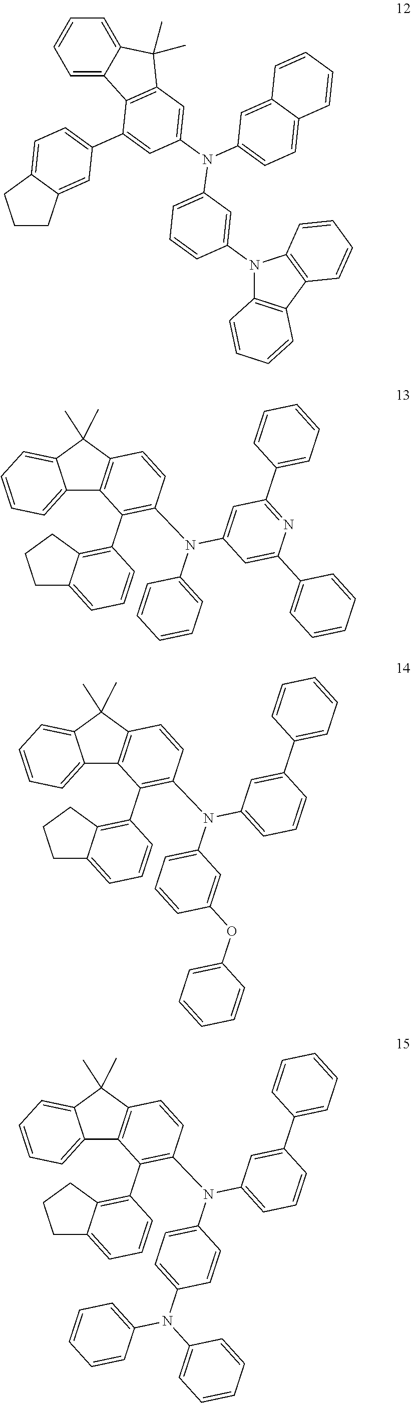

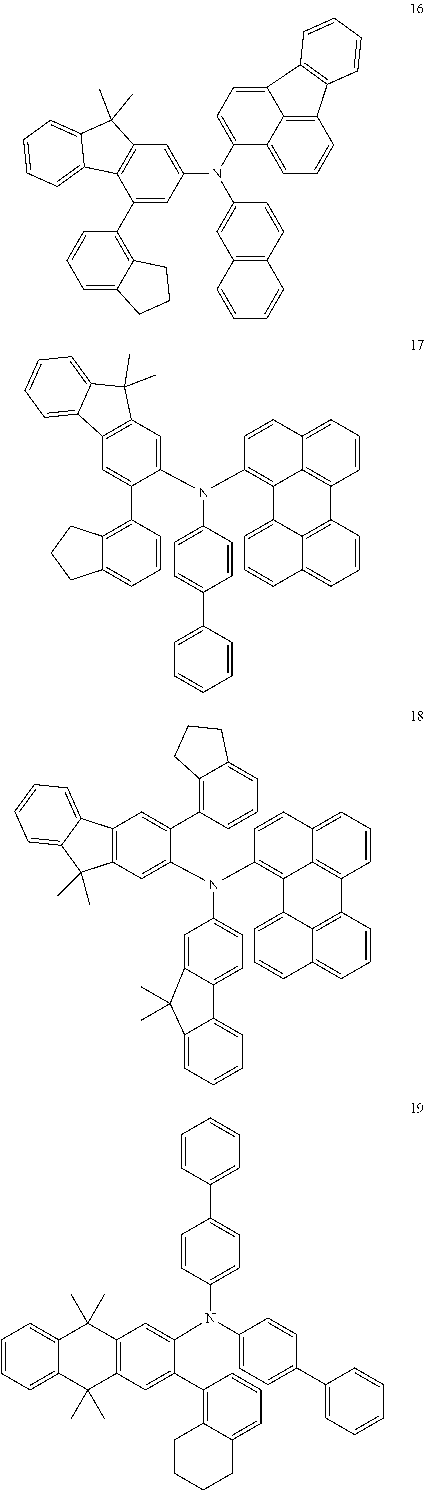

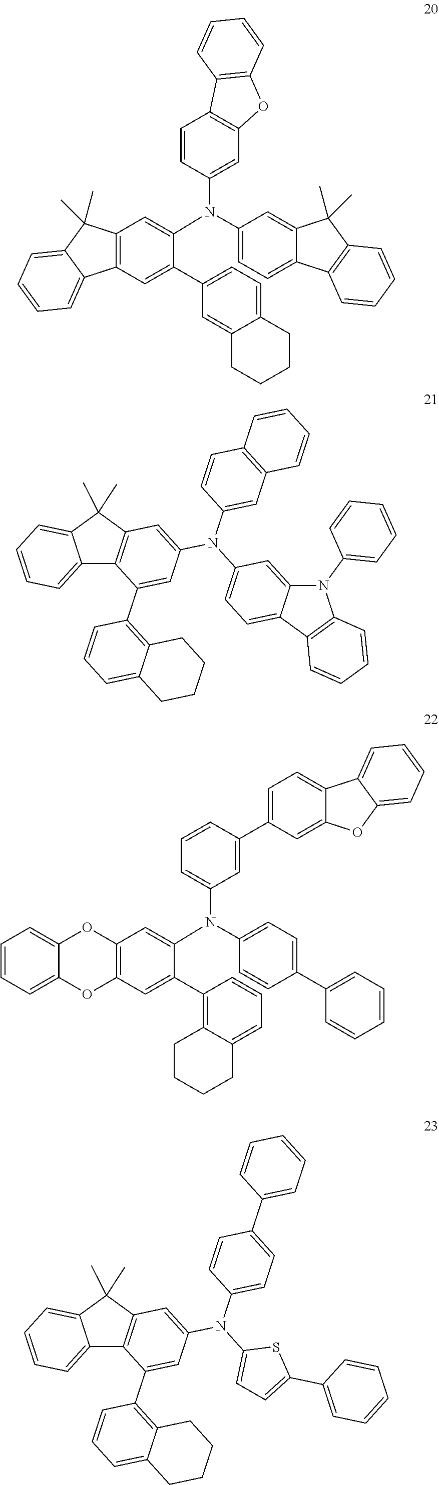

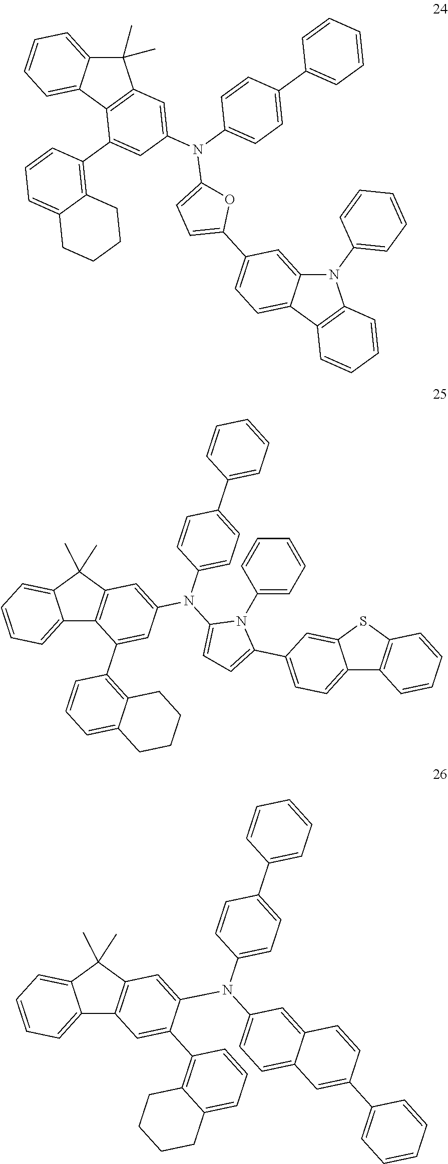 [SMILES: CC1(C)c2ccccc2-c2c(-c3cccc4c3CCCC4)cc(N(c3ccc(-c4ccccc4)cc3)c3ccc(-c4ccc5c6ccccc6n(-c6ccccc6)c5c4)o3)cc21 ; CC1(C)c2ccccc2-c2c(-c3cccc4c3CCCC4)cc(N(c3ccc(-c4ccccc4)cc3)c3ccc(-c4ccc5c(c4)sc4ccccc45)n3-c3ccccc3)cc21 ; CC1(C)c2ccccc2-c2cc(-c3cccc4c3CCCC4)c(N(c3ccc(-c4ccccc4)cc3)c3ccc4cc(-c5ccccc5)ccc4c3)cc21]

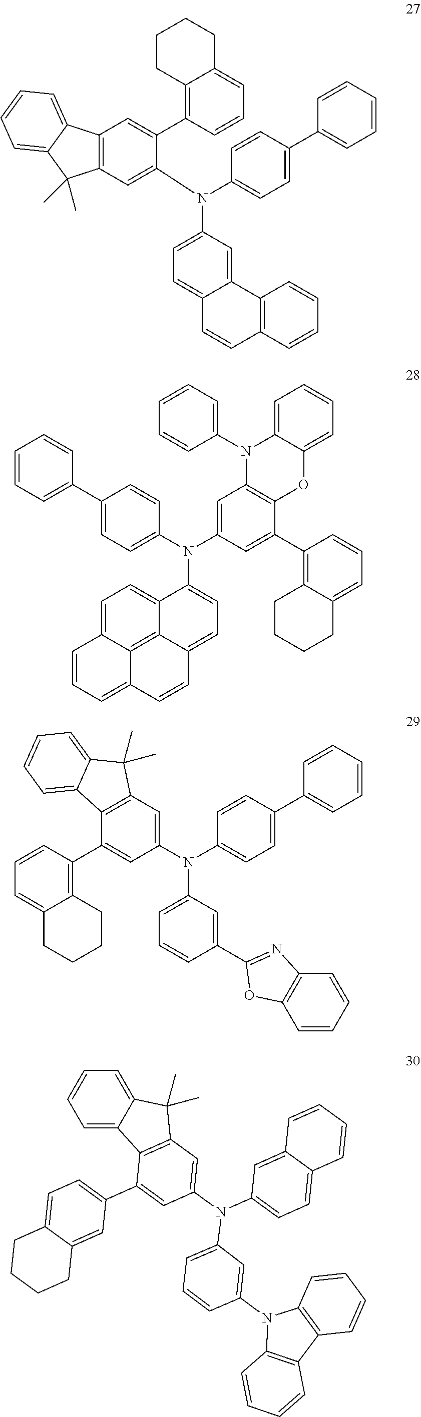

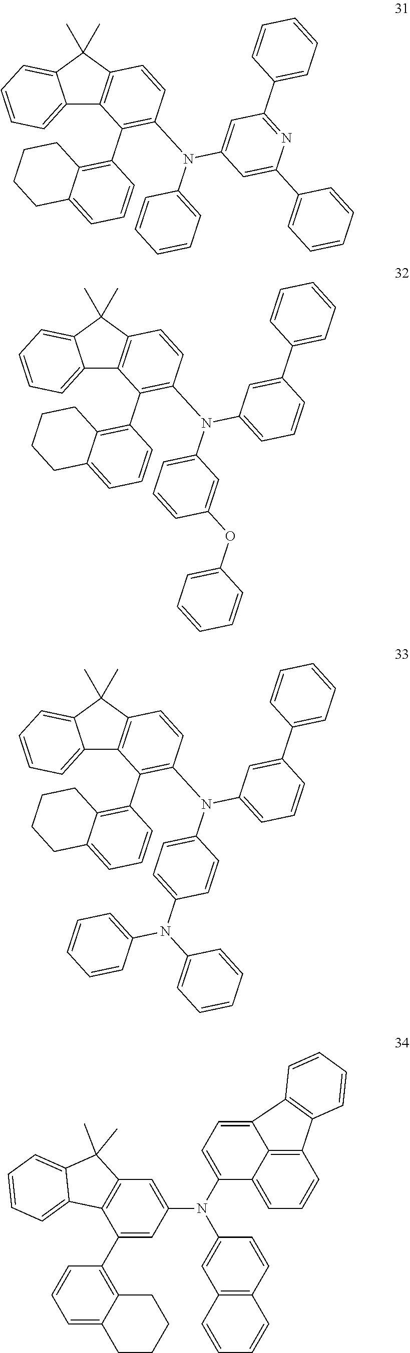

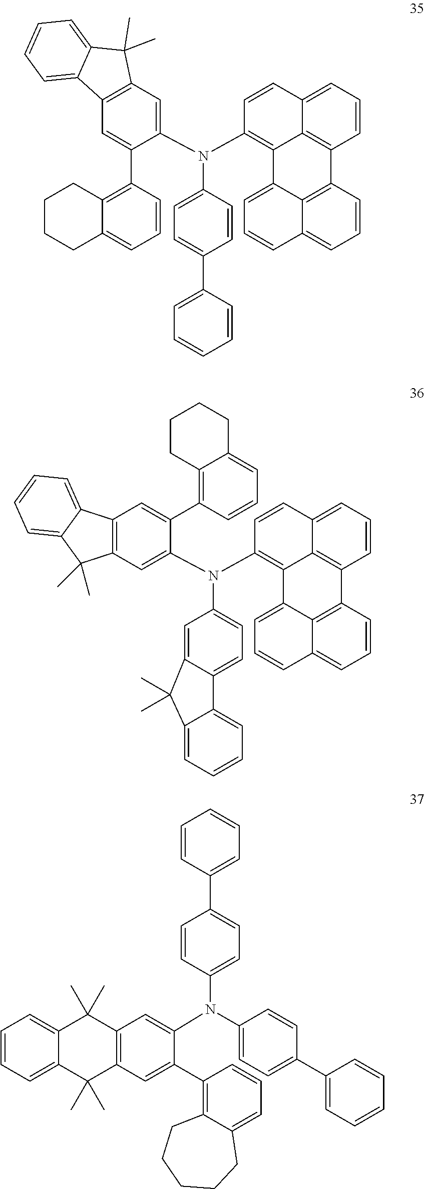

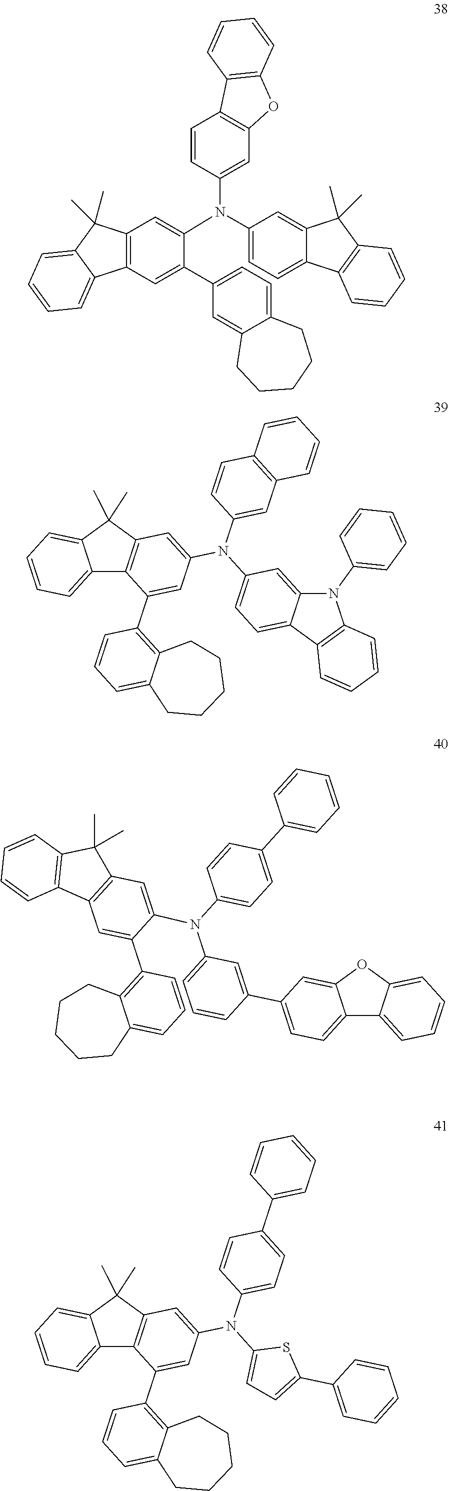

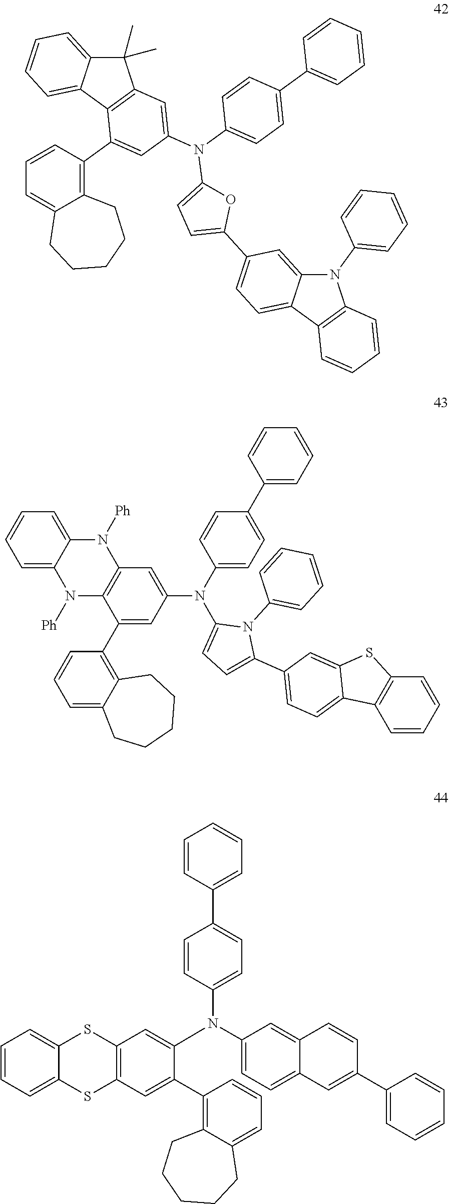 [SMILES: CC1(C)c2ccccc2-c2c(-c3cccc4c3CCCCC4)cc(N(c3ccc(-c4ccccc4)cc3)c3ccc(-c4ccc5c6ccccc6n(-c6ccccc6)c5c4)o3)cc21 ; c1ccc(-c2ccc(N(c3cc(-c4cccc5c4CCCCC5)c4c(c3)N(c3ccccc3)c3ccccc3N4c3ccccc3)c3ccc(-c4ccc5c(c4)sc4ccccc45)n3-c3ccccc3)cc2)cc1 ; c1ccc(-c2ccc(N(c3ccc4cc(-c5ccccc5)ccc4c3)c3cc4c(cc3-c3cccc5c3CCCCC5)Sc3ccccc3S4)cc2)cc1]

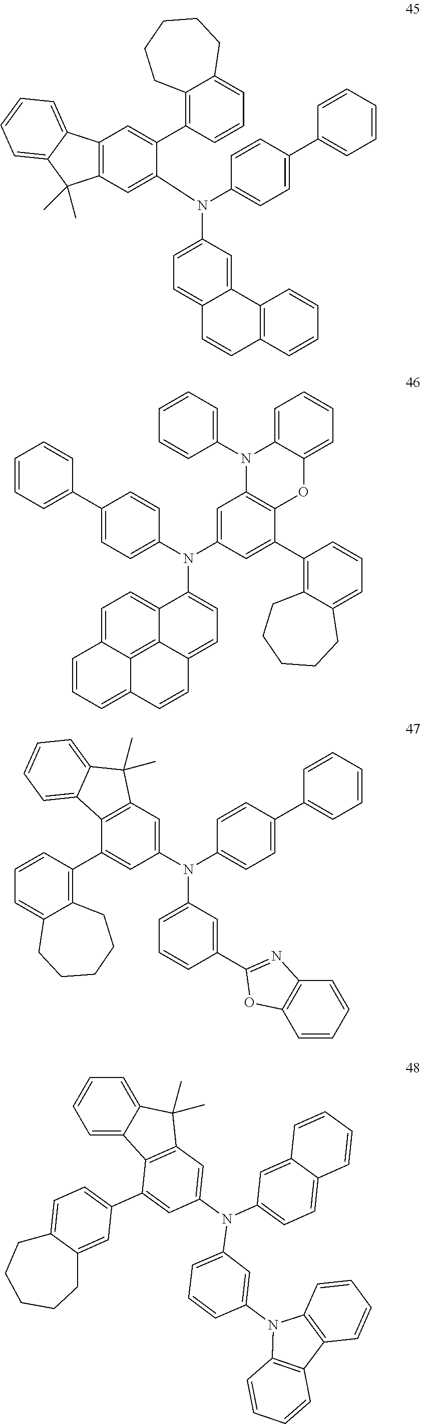

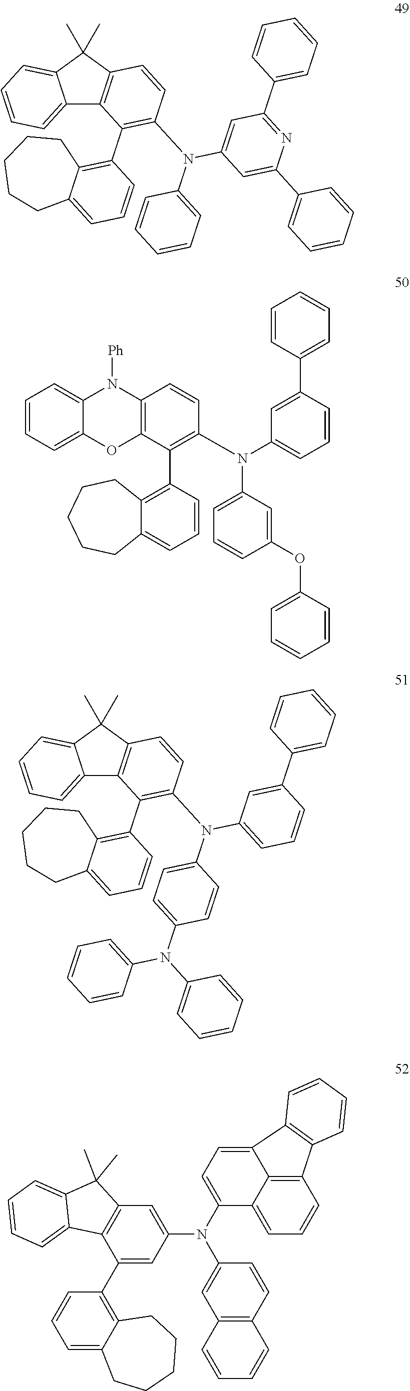

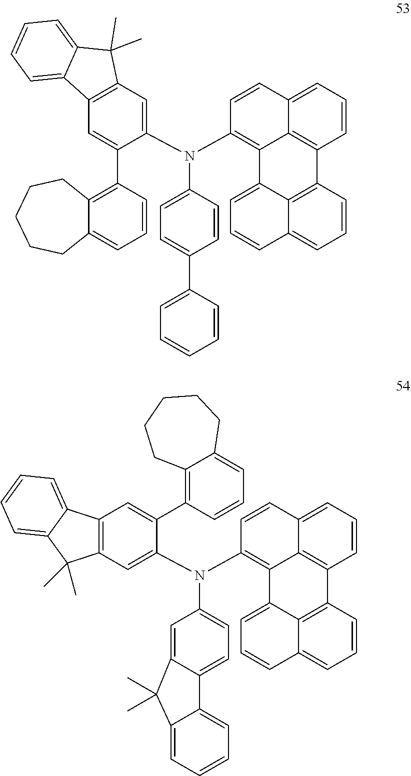

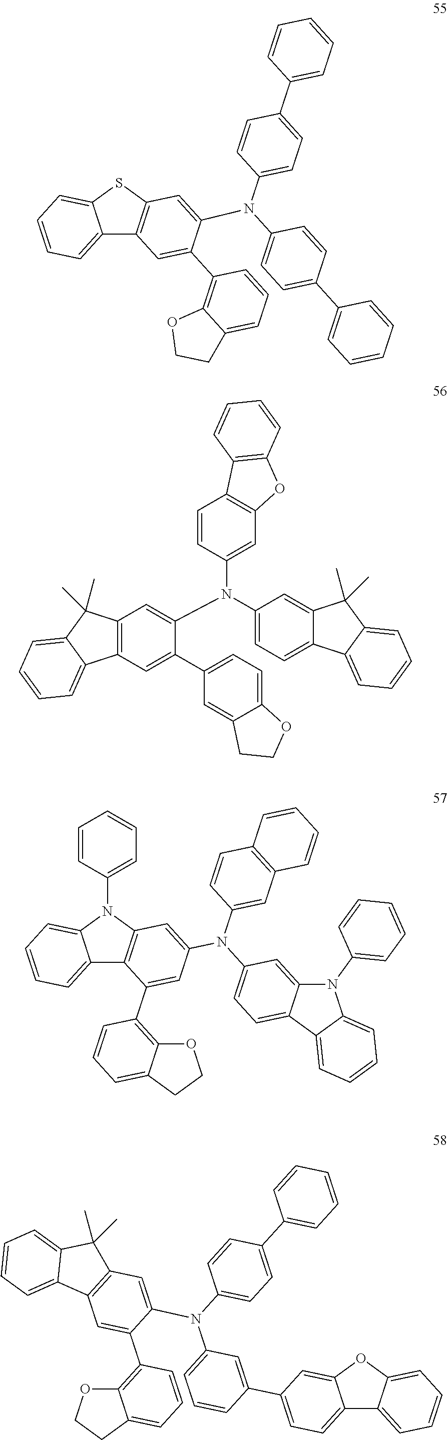

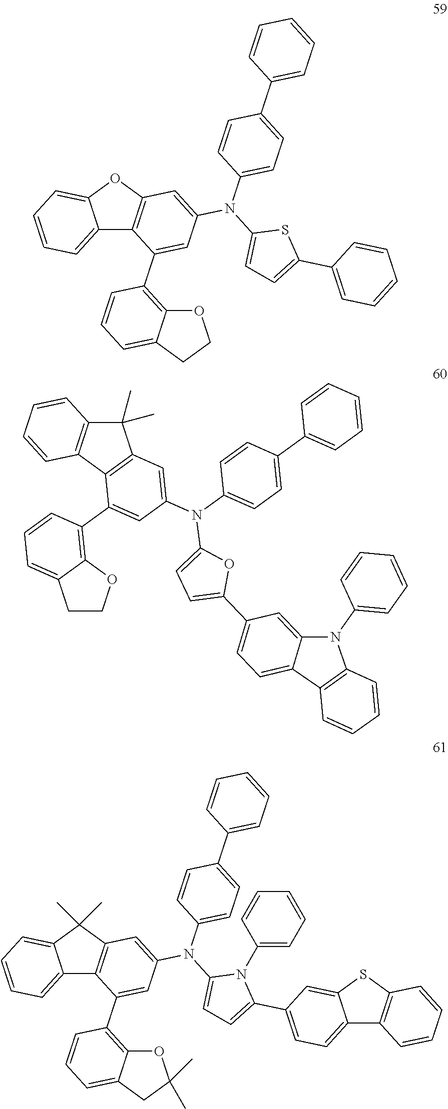

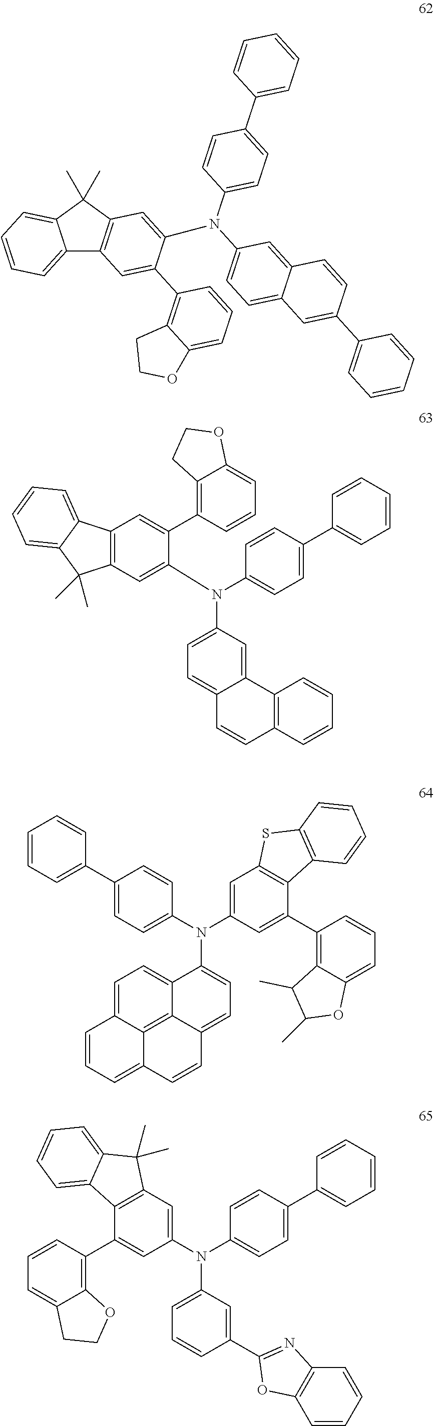 [SMILES: CC1(C)c2ccccc2-c2cc(-c3cccc4c3CCO4)c(N(c3ccc(-c4ccccc4)cc3)c3ccc4cc(-c5ccccc5)ccc4c3)cc21 ; CC1(C)c2ccccc2-c2cc(-c3cccc4c3CCO4)c(N(c3ccc(-c4ccccc4)cc3)c3ccc4ccc5ccccc5c4c3)cc21 ; CC1Oc2cccc(-c3cc(N(c4ccc(-c5ccccc5)cc4)c4ccc5ccc6cccc7ccc4c5c67)cc4sc5ccccc5c34)c2C1C ; CC1(C)c2ccccc2-c2c(-c3cccc4c3OCC4)cc(N(c3ccc(-c4ccccc4)cc3)c3cccc(-c4nc5ccccc5o4)c3)cc21]

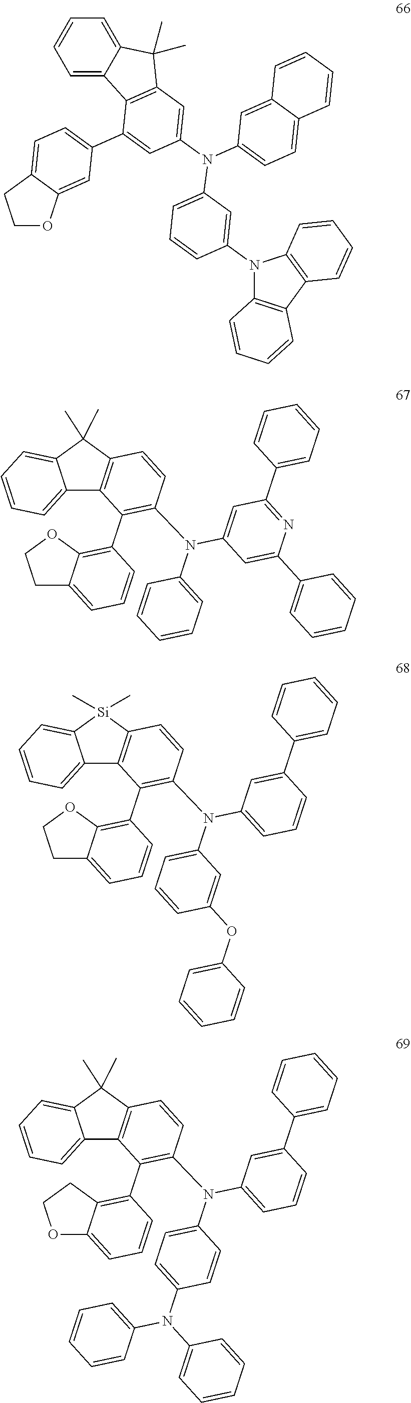

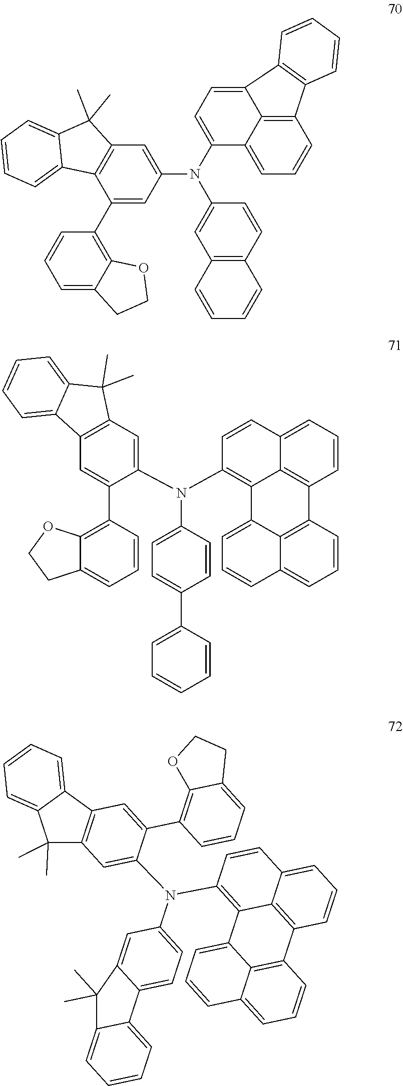

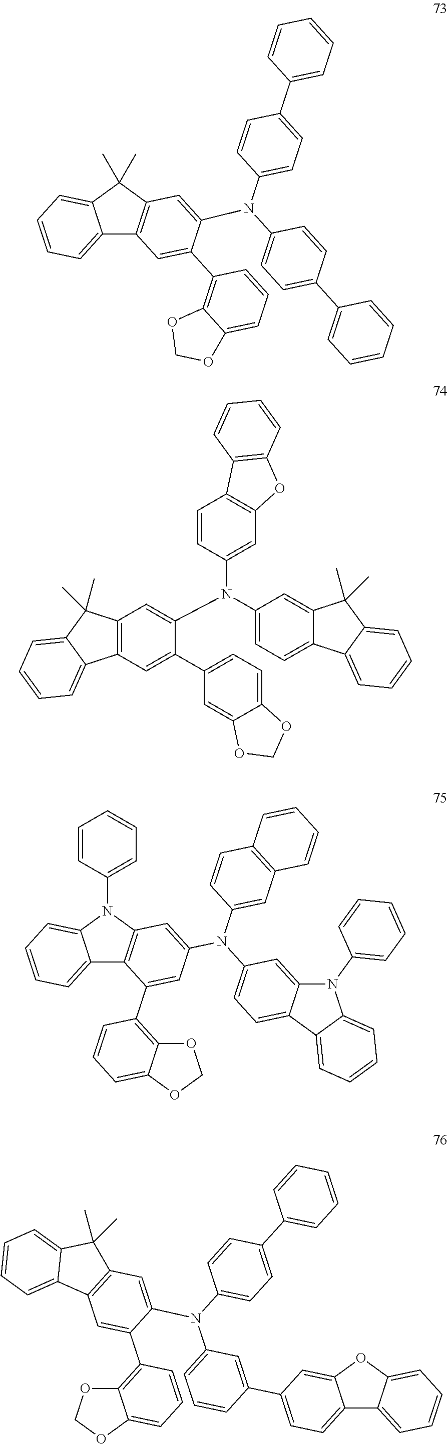

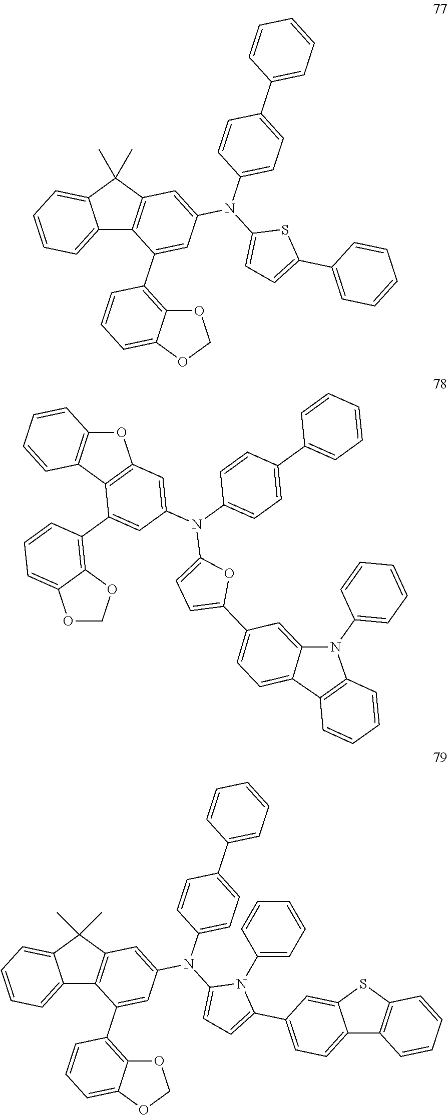 [SMILES: CC1(C)c2ccccc2-c2c(-c3cccc4c3OCO4)cc(N(c3ccc(-c4ccccc4)cc3)c3ccc(-c4ccccc4)s3)cc21 ; c1ccc(-c2ccc(N(c3cc(-c4cccc5c4OCO5)c4c(c3)oc3ccccc34)c3ccc(-c4ccc5c6ccccc6n(-c6ccccc6)c5c4)o3)cc2)cc1 ; CC1(C)c2ccccc2-c2c(-c3cccc4c3OCO4)cc(N(c3ccc(-c4ccccc4)cc3)c3ccc(-c4ccc5c(c4)sc4ccccc45)n3-c3ccccc3)cc21]

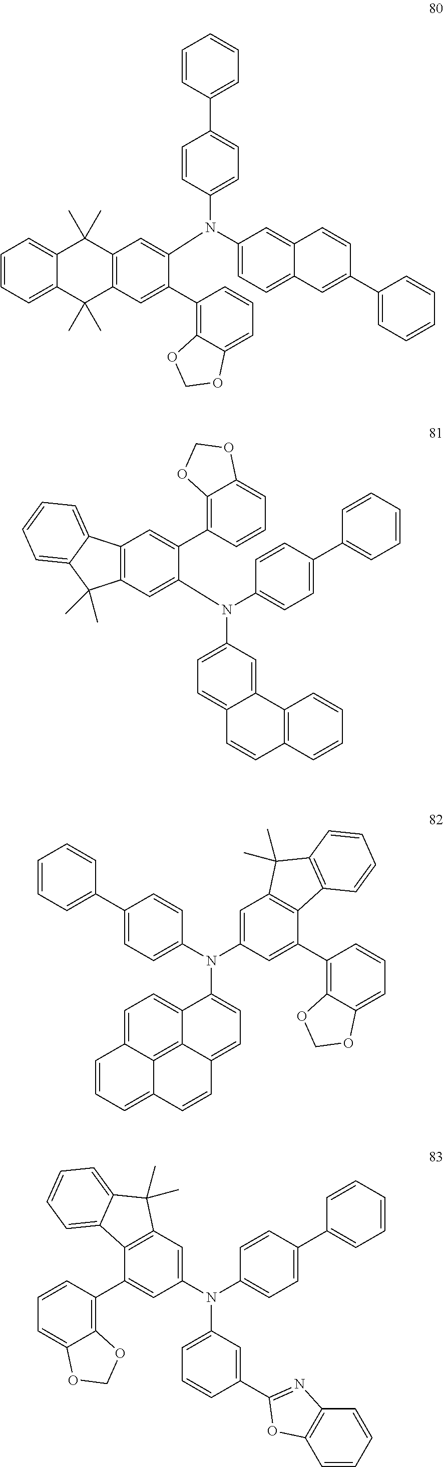 [SMILES: CC1(C)c2ccccc2C(C)(C)c2cc(N(c3ccc(-c4ccccc4)cc3)c3ccc4cc(-c5ccccc5)ccc4c3)c(-c3cccc4c3OCO4)cc21 ; CC1(C)c2ccccc2-c2cc(-c3cccc4c3OCO4)c(N(c3ccc(-c4ccccc4)cc3)c3ccc4ccc5ccccc5c4c3)cc21 ; CC1(C)c2ccccc2-c2c(-c3cccc4c3OCO4)cc(N(c3ccc(-c4ccccc4)cc3)c3ccc4ccc5cccc6ccc3c4c56)cc21 ; CC1(C)c2ccccc2-c2c(-c3cccc4c3OCO4)cc(N(c3ccc(-c4ccccc4)cc3)c3cccc(-c4nc5ccccc5o4)c3)cc21]

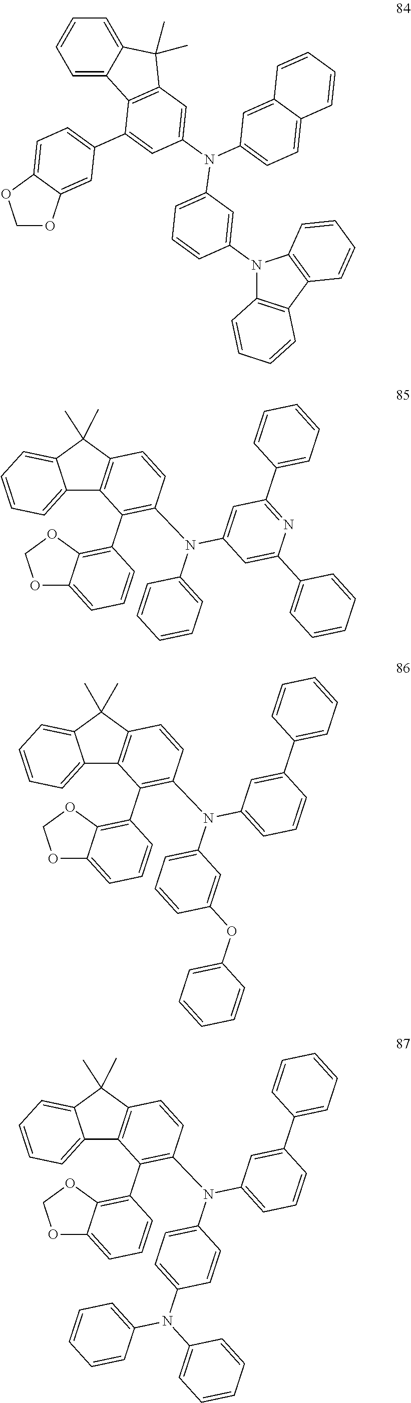

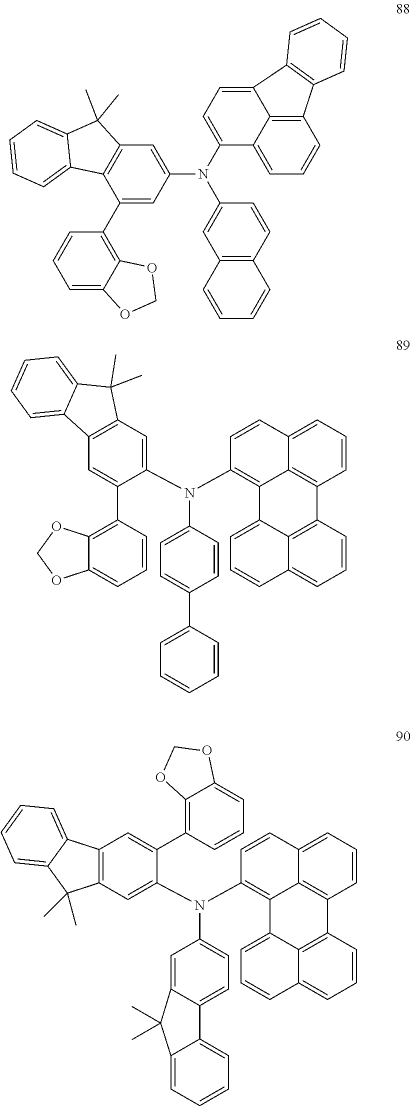

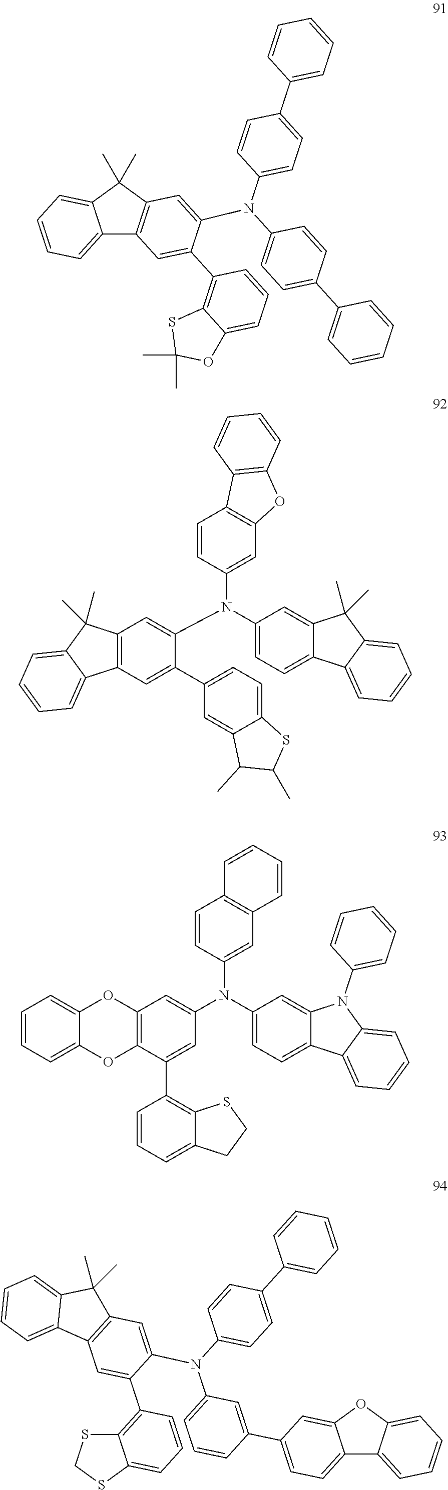 [SMILES: CC1(C)Oc2cccc(-c3cc4c(cc3N(c3ccc(-c5ccccc5)cc3)c3ccc(-c5ccccc5)cc3)C(C)(C)c3ccccc3-4)c2S1 ; CC1Sc2ccc(-c3cc4c(cc3N(c3ccc5c(c3)C(C)(C)c3ccccc3-5)c3ccc5c(c3)oc3ccccc35)C(C)(C)c3ccccc3-4)cc2C1C ; c1ccc(-n2c3ccccc3c3ccc(N(c4cc5c(c(-c6cccc7c6SCC7)c4)Oc4ccccc4O5)c4ccc5ccccc5c4)cc32)cc1 ; CC1(C)c2ccccc2-c2cc(-c3cccc4c3SCS4)c(N(c3ccc(-c4ccccc4)cc3)c3cccc(-c4ccc5c(c4)oc4ccccc45)c3)cc21]

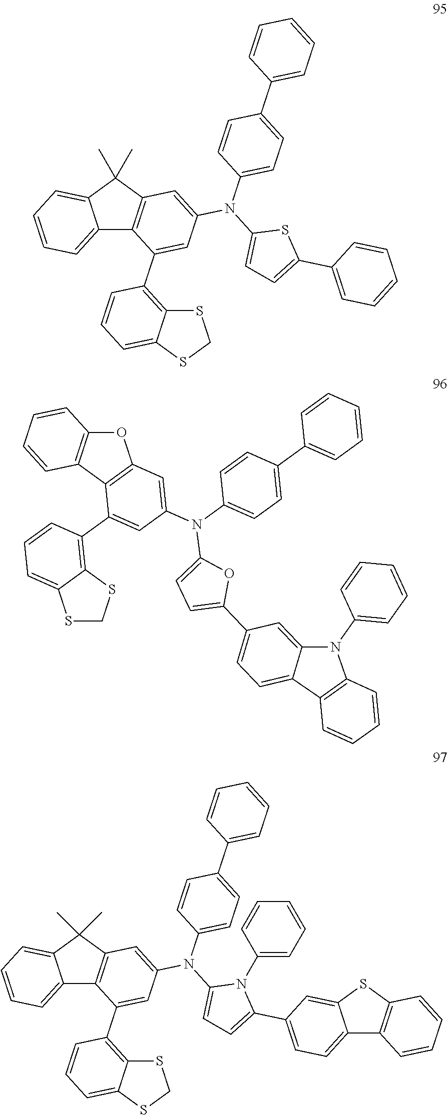 [SMILES: CC1(C)c2ccccc2-c2c(-c3cccc4c3SCS4)cc(N(c3ccc(-c4ccccc4)cc3)c3ccc(-c4ccccc4)s3)cc21 ; c1ccc(-c2ccc(N(c3cc(-c4cccc5c4SCS5)c4c(c3)oc3ccccc34)c3ccc(-c4ccc5c6ccccc6n(-c6ccccc6)c5c4)o3)cc2)cc1 ; CC1(C)c2ccccc2-c2c(-c3cccc4c3SCS4)cc(N(c3ccc(-c4ccccc4)cc3)c3ccc(-c4ccc5c(c4)sc4ccccc45)n3-c3ccccc3)cc21]

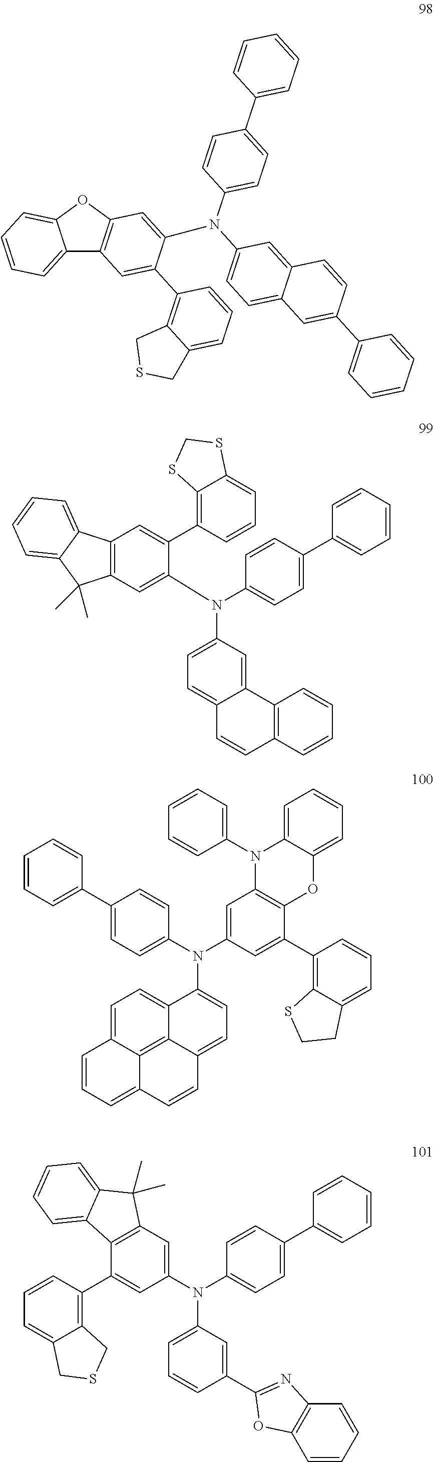

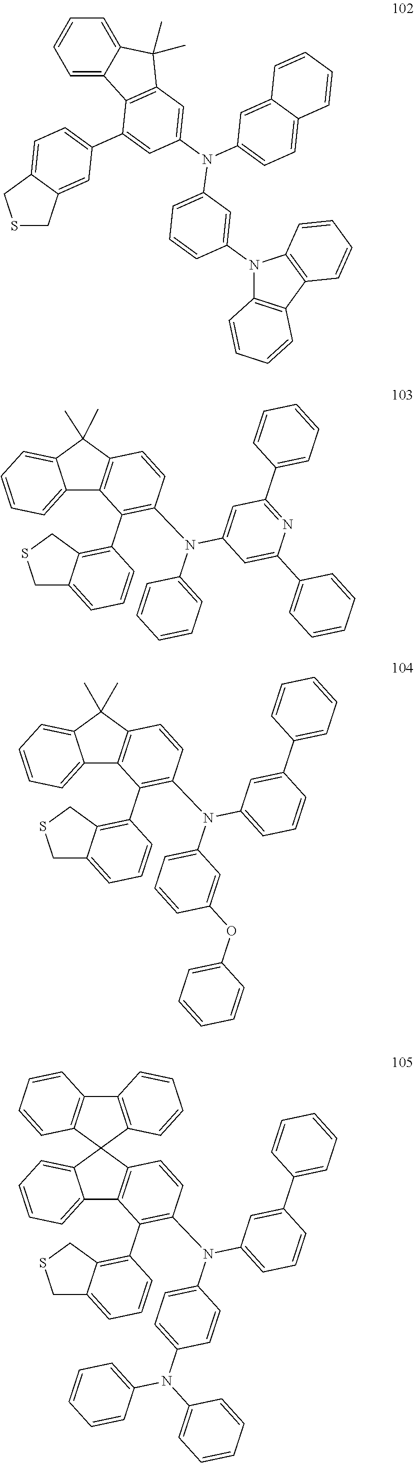

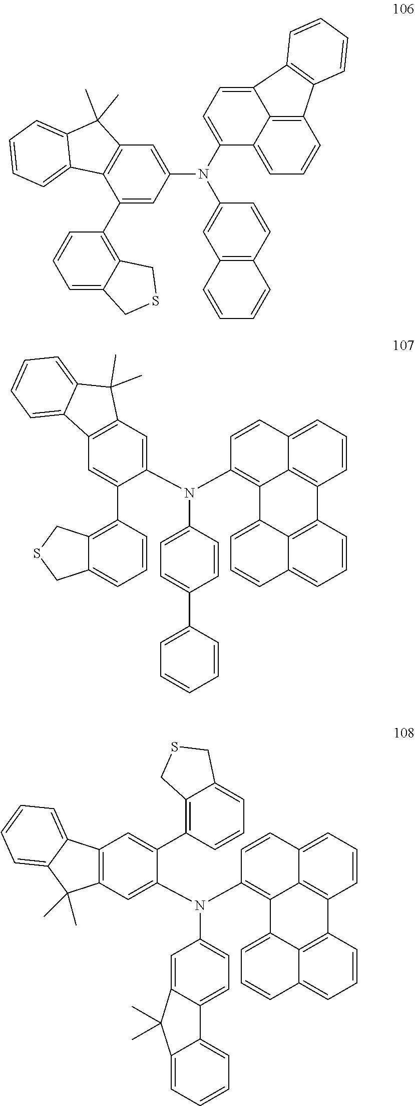

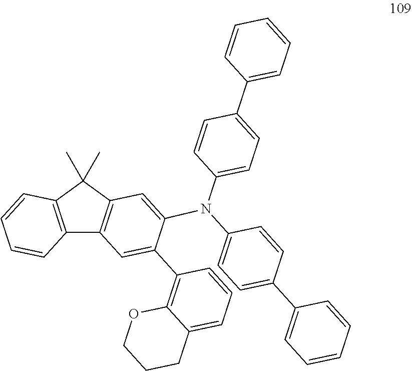 [SMILES: CC1(C)c2ccccc2-c2cc(-c3cccc4c3OCCC4)c(N(c3ccc(-c4ccccc4)cc3)c3ccc(-c4ccccc4)cc3)cc21]

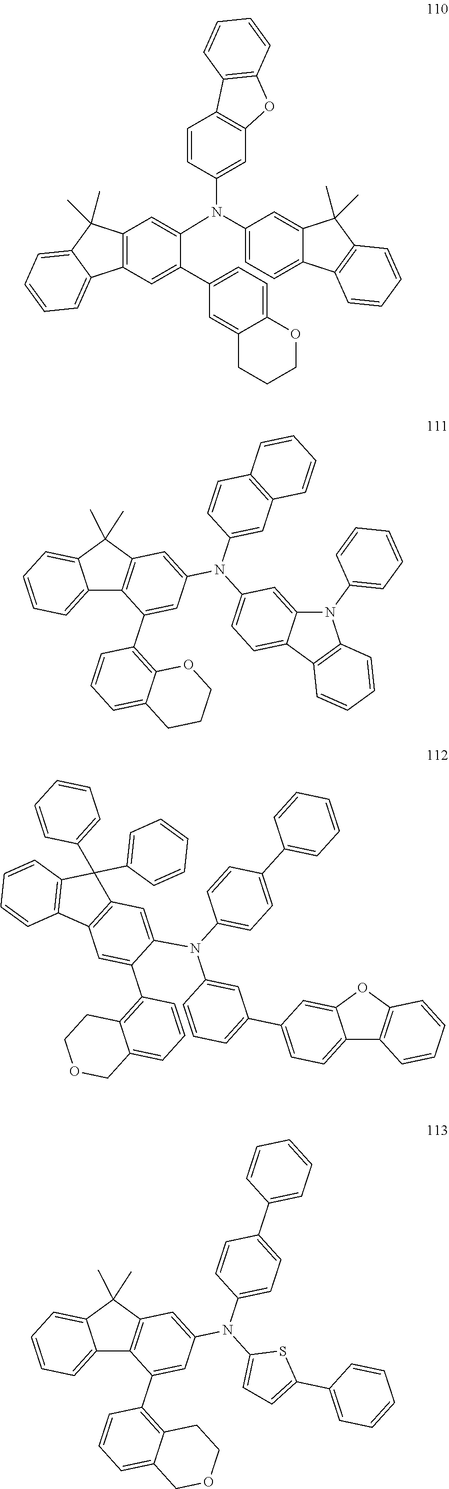 [SMILES: CC1(C)c2ccccc2-c2ccc(N(c3ccc4c(c3)oc3ccccc34)c3cc4c(cc3-c3ccc5c(c3)CCCO5)-c3ccccc3C4(C)C)cc21 ; CC1(C)c2ccccc2-c2c(-c3cccc4c3OCCC4)cc(N(c3ccc4ccccc4c3)c3ccc4c5ccccc5n(-c5ccccc5)c4c3)cc21 ; c1ccc(-c2ccc(N(c3cccc(-c4ccc5c(c4)oc4ccccc45)c3)c3cc4c(cc3-c3cccc5c3CCOC5)-c3ccccc3C4(c3ccccc3)c3ccccc3)cc2)cc1 ; CC1(C)c2ccccc2-c2c(-c3cccc4c3CCOC4)cc(N(c3ccc(-c4ccccc4)cc3)c3ccc(-c4ccccc4)s3)cc21]

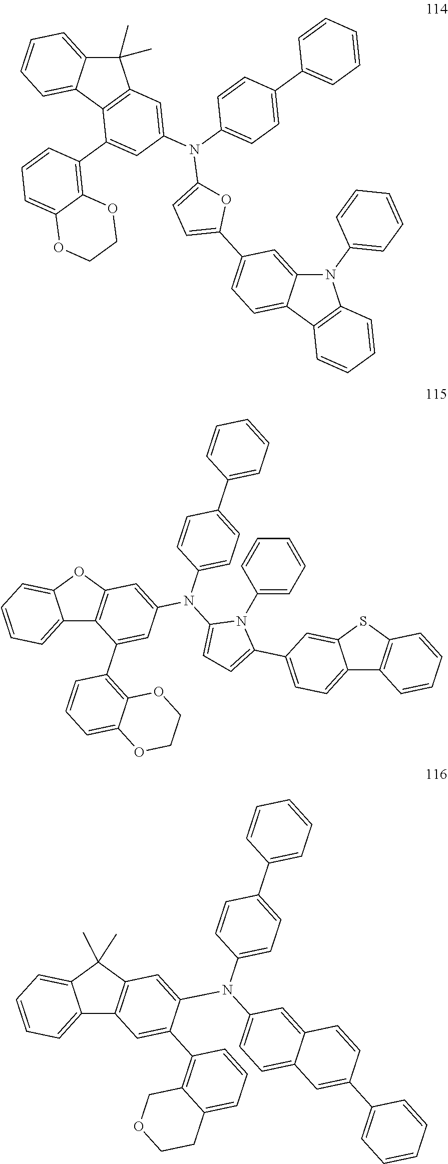 [SMILES: CC1(C)c2ccccc2-c2c(-c3cccc4c3OCCO4)cc(N(c3ccc(-c4ccccc4)cc3)c3ccc(-c4ccc5c6ccccc6n(-c6ccccc6)c5c4)o3)cc21 ; c1ccc(-c2ccc(N(c3cc(-c4cccc5c4OCCO5)c4c(c3)oc3ccccc34)c3ccc(-c4ccc5c(c4)sc4ccccc45)n3-c3ccccc3)cc2)cc1 ; CC1(C)c2ccccc2-c2cc(-c3cccc4c3COCC4)c(N(c3ccc(-c4ccccc4)cc3)c3ccc4cc(-c5ccccc5)ccc4c3)cc21]

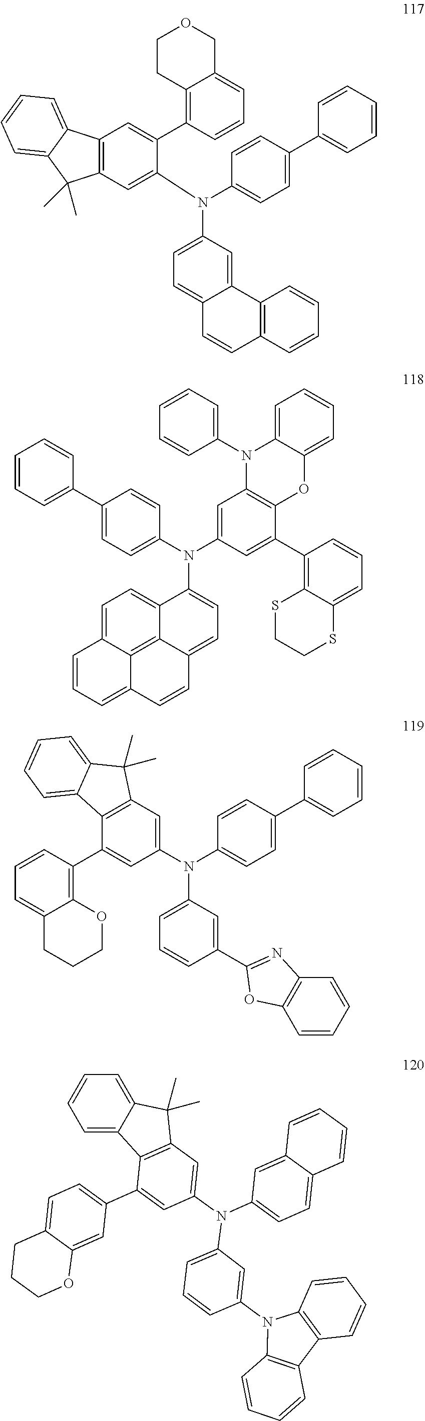

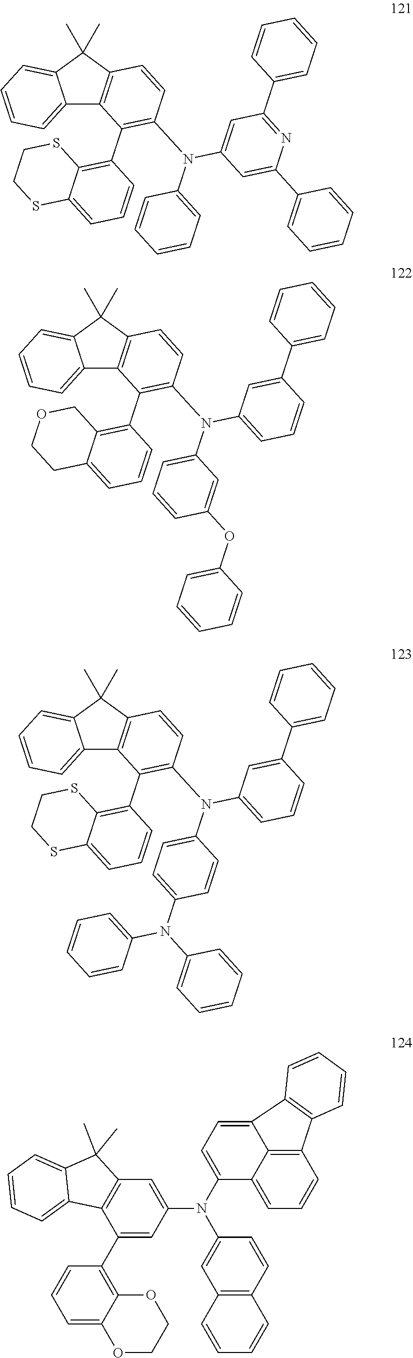

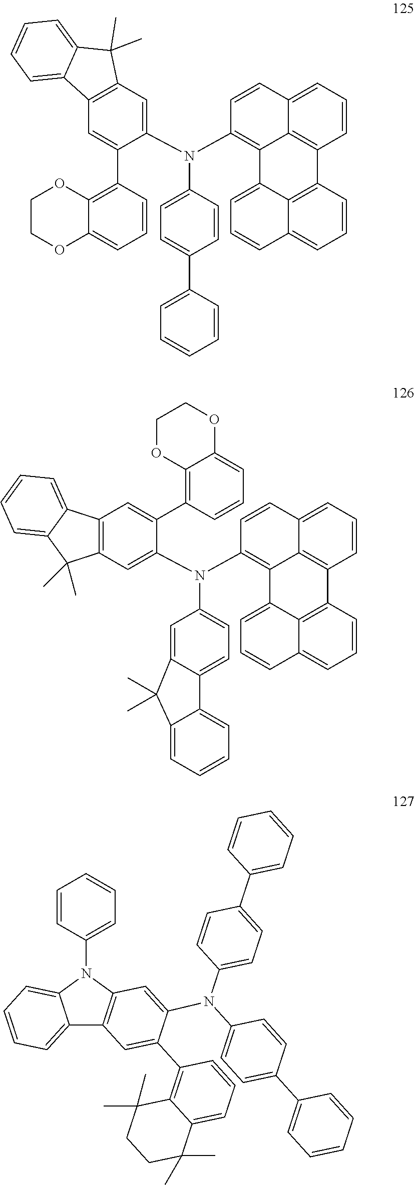 [SMILES: CC1(C)c2ccccc2-c2cc(-c3cccc4c3OCCO4)c(N(c3ccc(-c4ccccc4)cc3)c3ccc4cccc5c6cccc7cccc(c3c45)c76)cc21 ; CC1(C)c2ccccc2-c2ccc(N(c3cc4c(cc3-c3cccc5c3OCCO5)-c3ccccc3C4(C)C)c3ccc4cccc5c6cccc7cccc(c3c45)c76)cc21 ; CC1(C)CCC(C)(C)c2c(-c3cc4c5ccccc5n(-c5ccccc5)c4cc3N(c3ccc(-c4ccccc4)cc3)c3ccc(-c4ccccc4)cc3)cccc21]

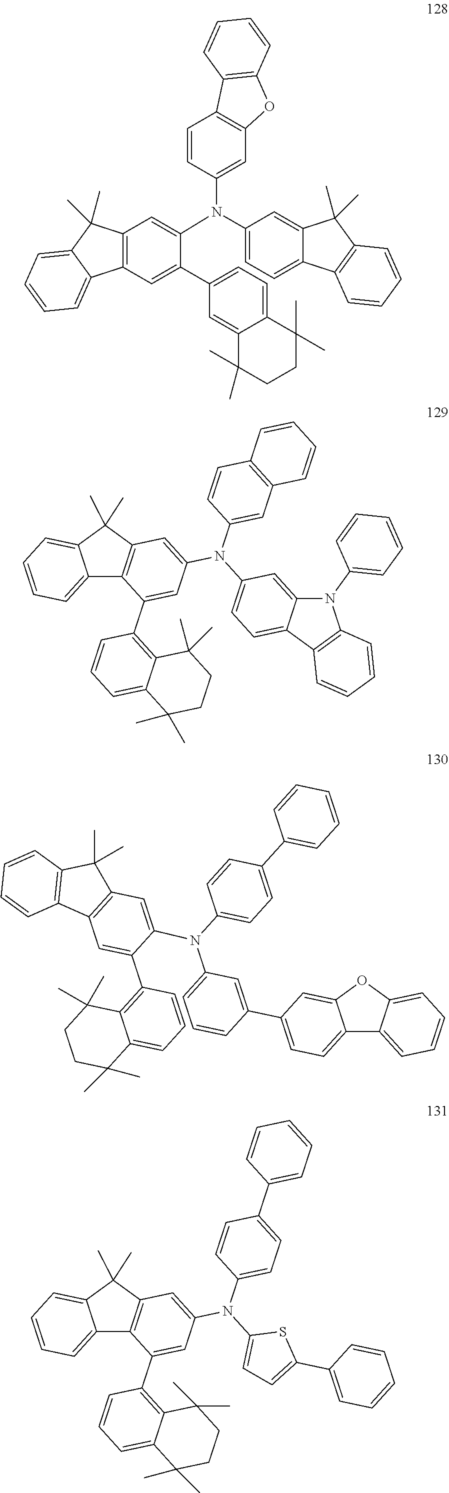

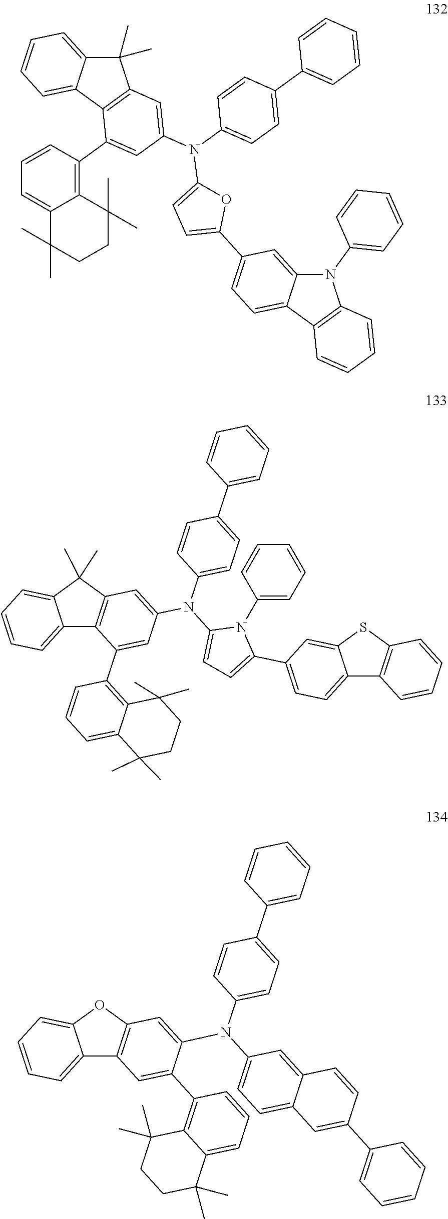 [SMILES: CC1(C)CCC(C)(C)c2c(-c3cc(N(c4ccc(-c5ccccc5)cc4)c4ccc(-c5ccc6c7ccccc7n(-c7ccccc7)c6c5)o4)cc4c3-c3ccccc3C4(C)C)cccc21 ; CC1(C)CCC(C)(C)c2c(-c3cc(N(c4ccc(-c5ccccc5)cc4)c4ccc(-c5ccc6c(c5)sc5ccccc56)n4-c4ccccc4)cc4c3-c3ccccc3C4(C)C)cccc21 ; CC1(C)CCC(C)(C)c2c(-c3cc4c(cc3N(c3ccc(-c5ccccc5)cc3)c3ccc5cc(-c6ccccc6)ccc5c3)oc3ccccc34)cccc21]

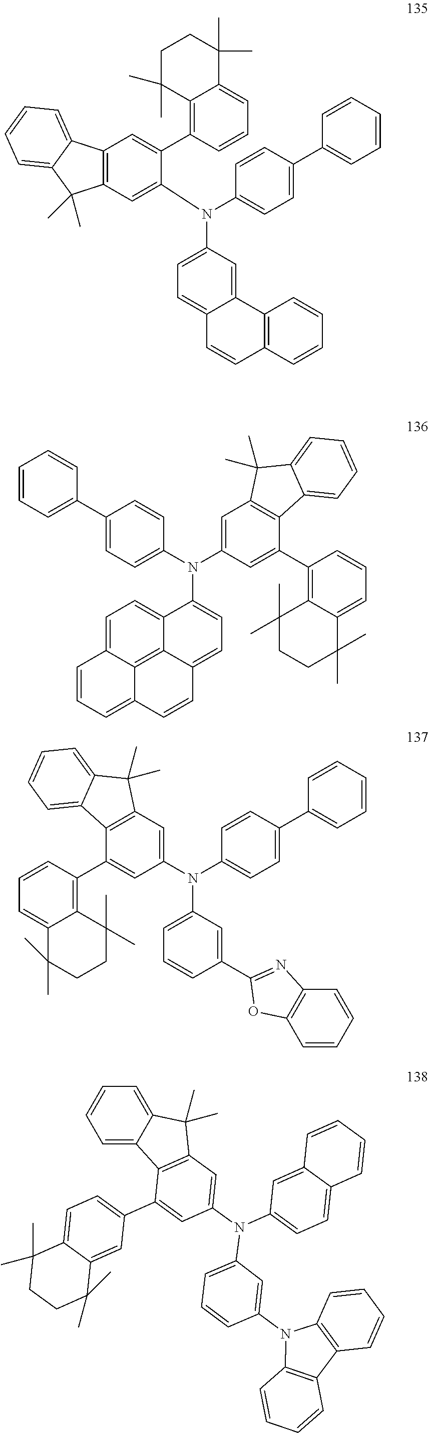

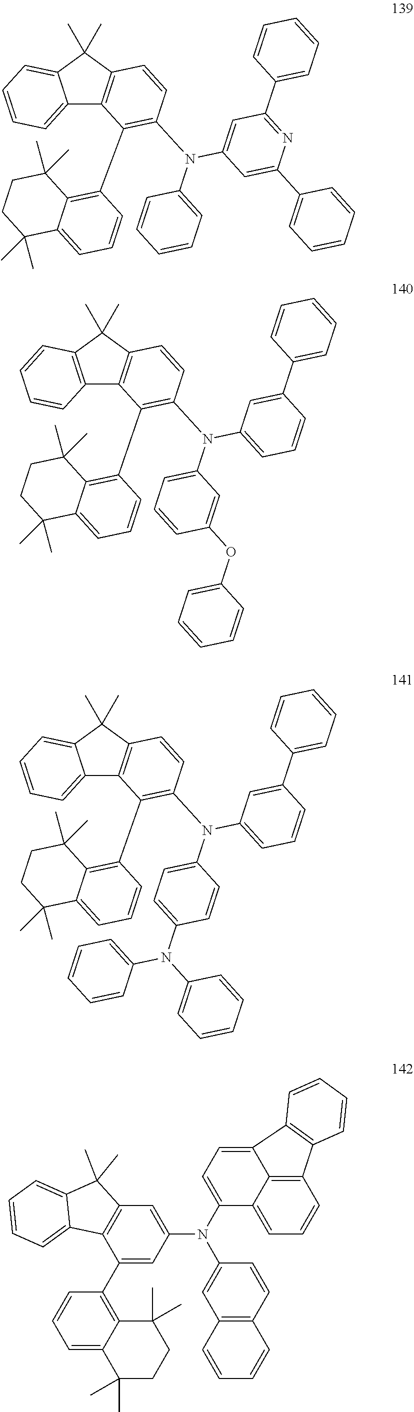 [SMILES: CC1(C)CCC(C)(C)c2c(-c3c(N(c4ccccc4)c4cc(-c5ccccc5)nc(-c5ccccc5)c4)ccc4c3-c3ccccc3C4(C)C)cccc21 ; CC1(C)CCC(C)(C)c2c(-c3c(N(c4cccc(Oc5ccccc5)c4)c4cccc(-c5ccccc5)c4)ccc4c3-c3ccccc3C4(C)C)cccc21 ; CC1(C)CCC(C)(C)c2c(-c3c(N(c4ccc(N(c5ccccc5)c5ccccc5)cc4)c4cccc(-c5ccccc5)c4)ccc4c3-c3ccccc3C4(C)C)cccc21 ; CC1(C)CCC(C)(C)c2c(-c3cc(N(c4ccc5ccccc5c4)c4ccc5c6c(cccc46)-c4ccccc4-5)cc4c3-c3ccccc3C4(C)C)cccc21]

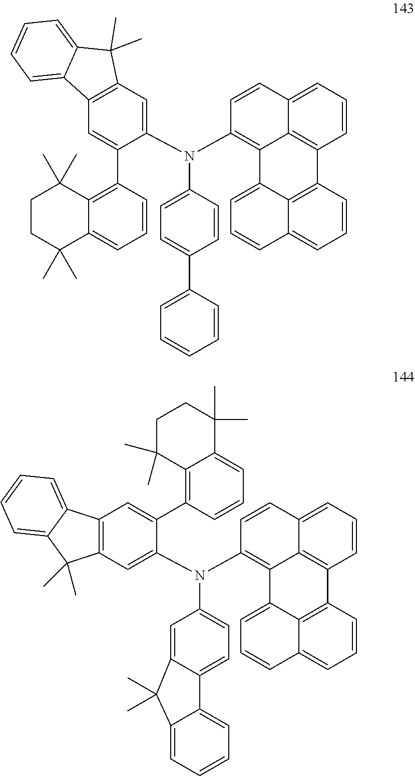

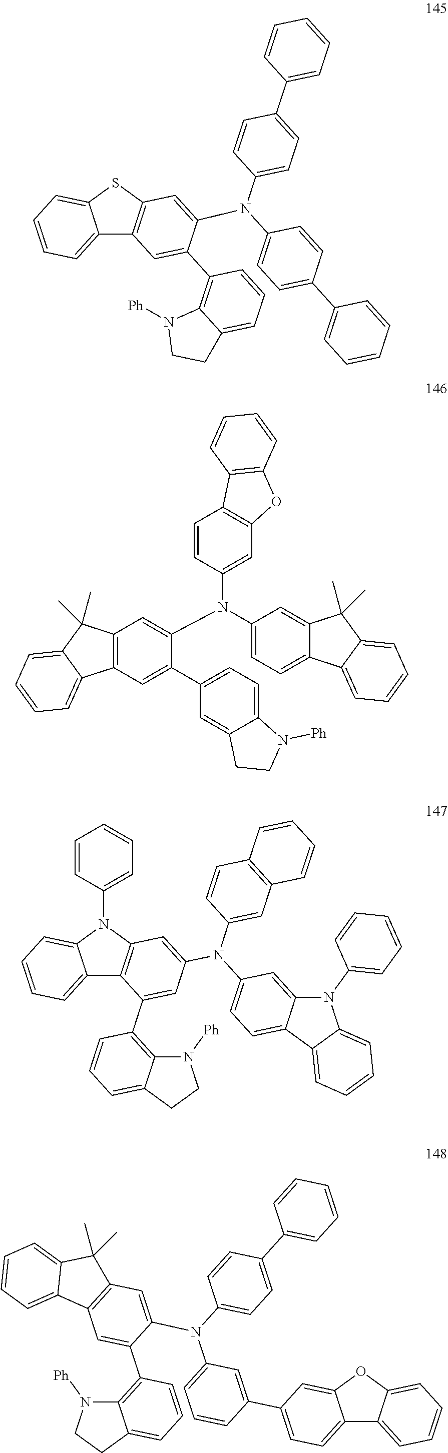 [SMILES: c1ccc(-c2ccc(N(c3ccc(-c4ccccc4)cc3)c3cc4sc5ccccc5c4cc3-c3cccc4c3N(c3ccccc3)CC4)cc2)cc1 ; CC1(C)c2ccccc2-c2ccc(N(c3ccc4c(c3)oc3ccccc34)c3cc4c(cc3-c3ccc5c(c3)CCN5c3ccccc3)-c3ccccc3C4(C)C)cc21 ; c1ccc(N2CCc3cccc(-c4cc(N(c5ccc6ccccc6c5)c5ccc6c7ccccc7n(-c7ccccc7)c6c5)cc5c4c4ccccc4n5-c4ccccc4)c32)cc1 ; CC1(C)c2ccccc2-c2cc(-c3cccc4c3N(c3ccccc3)CC4)c(N(c3ccc(-c4ccccc4)cc3)c3cccc(-c4ccc5c(c4)oc4ccccc45)c3)cc21]

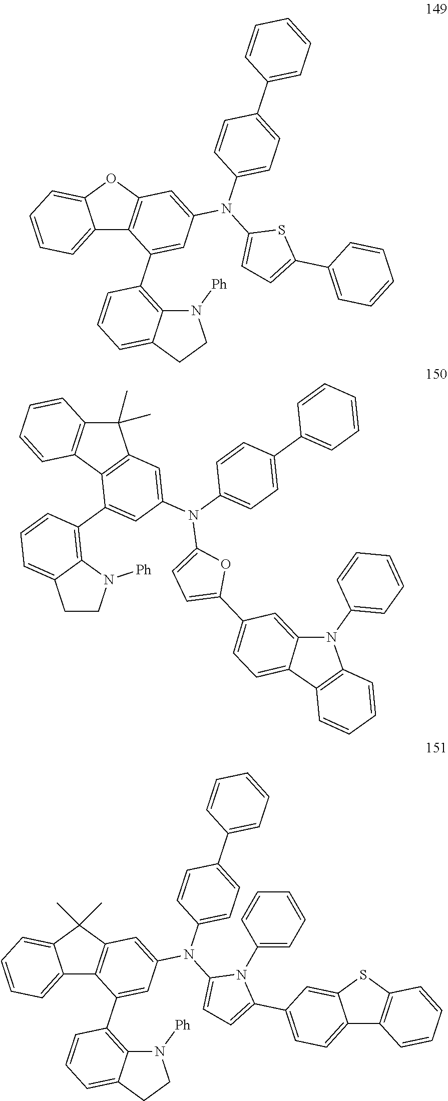

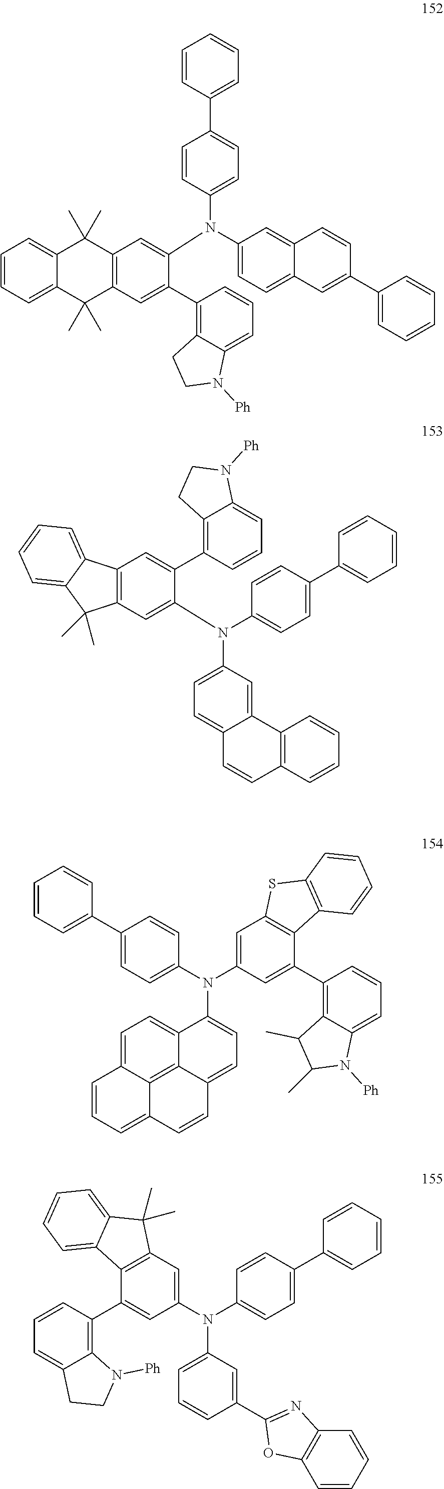

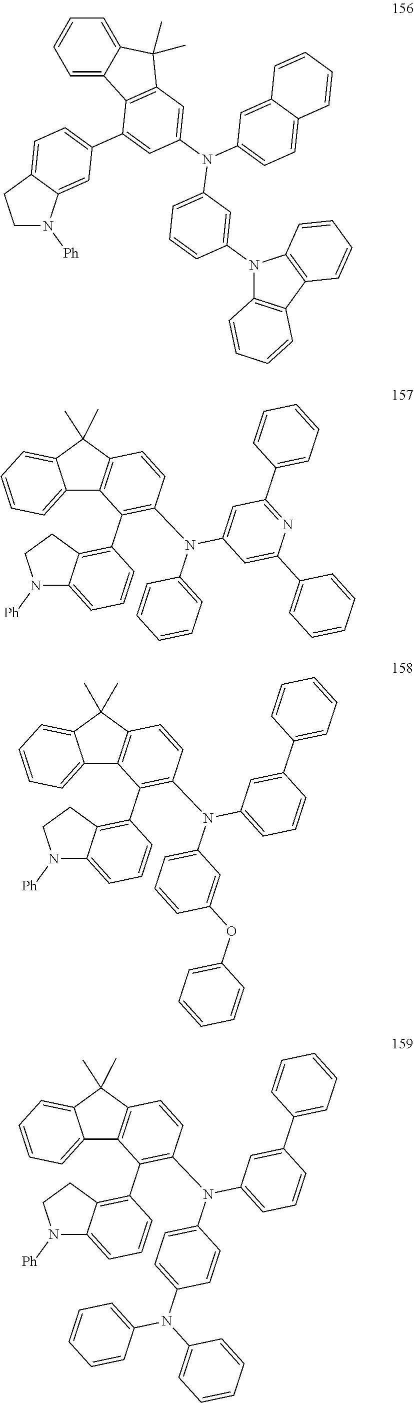

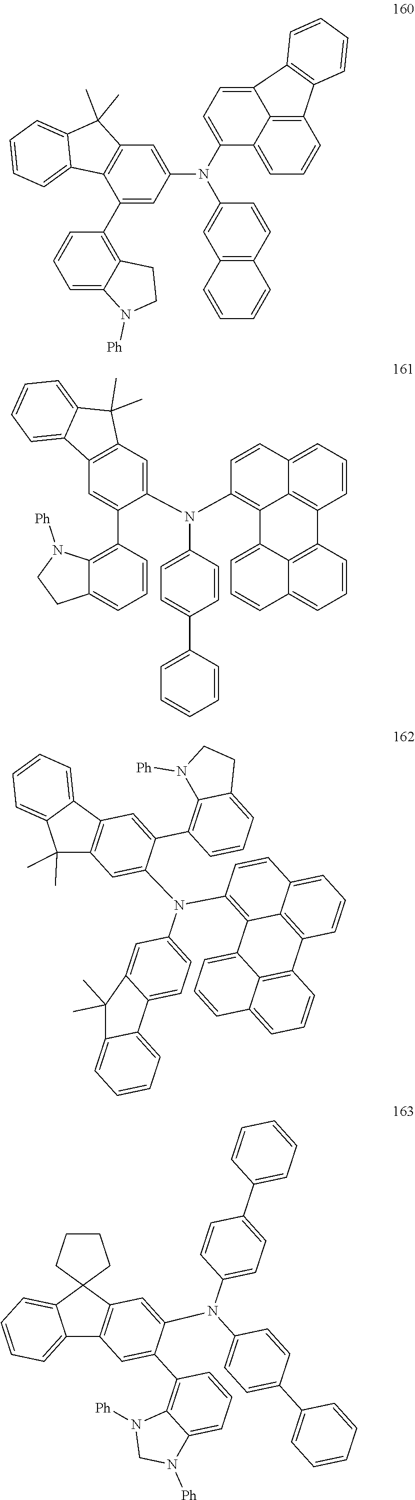

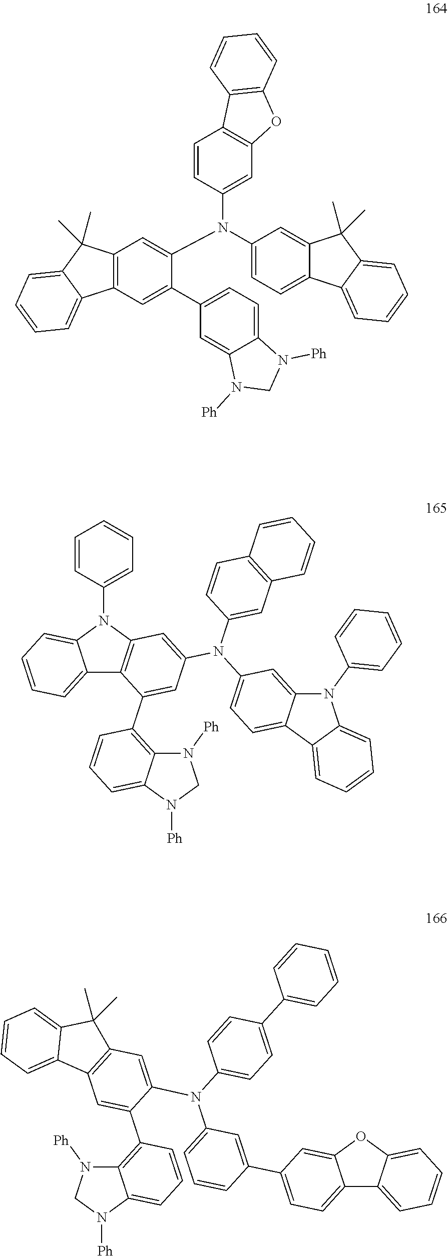 [SMILES: CC1(C)c2ccccc2-c2ccc(N(c3ccc4c(c3)oc3ccccc34)c3cc4c(cc3-c3ccc5c(c3)N(c3ccccc3)CN5c3ccccc3)-c3ccccc3C4(C)C)cc21 ; c1ccc(N2CN(c3ccccc3)c3c(-c4cc(N(c5ccc6ccccc6c5)c5ccc6c7ccccc7n(-c7ccccc7)c6c5)cc5c4c4ccccc4n5-c4ccccc4)cccc32)cc1 ; CC1(C)c2ccccc2-c2cc(-c3cccc4c3N(c3ccccc3)CN4c3ccccc3)c(N(c3ccc(-c4ccccc4)cc3)c3cccc(-c4ccc5c(c4)oc4ccccc45)c3)cc21]

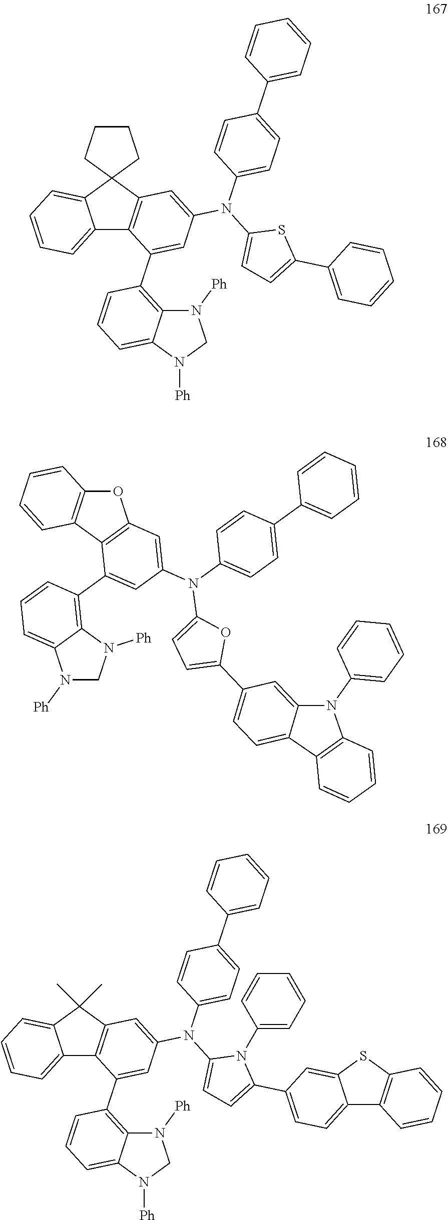 [SMILES: c1ccc(-c2ccc(N(c3cc(-c4cccc5c4N(c4ccccc4)CN5c4ccccc4)c4c(c3)C3(CCCC3)c3ccccc3-4)c3ccc(-c4ccccc4)s3)cc2)cc1 ; c1ccc(-c2ccc(N(c3cc(-c4cccc5c4N(c4ccccc4)CN5c4ccccc4)c4c(c3)oc3ccccc34)c3ccc(-c4ccc5c6ccccc6n(-c6ccccc6)c5c4)o3)cc2)cc1 ; CC1(C)c2ccccc2-c2c(-c3cccc4c3N(c3ccccc3)CN4c3ccccc3)cc(N(c3ccc(-c4ccccc4)cc3)c3ccc(-c4ccc5c(c4)sc4ccccc45)n3-c3ccccc3)cc21]

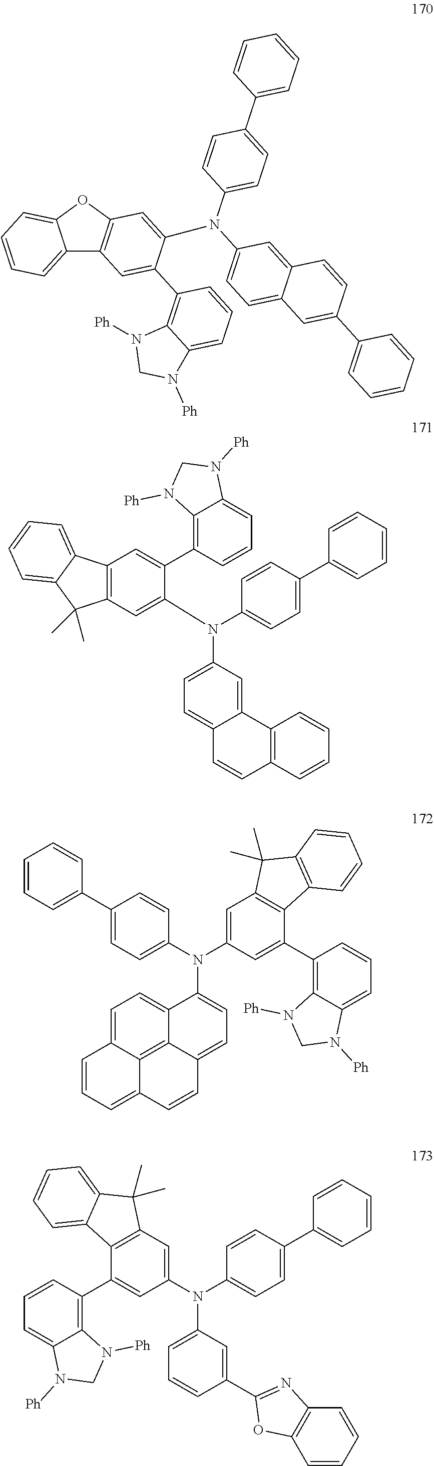 [SMILES: c1ccc(-c2ccc(N(c3ccc4cc(-c5ccccc5)ccc4c3)c3cc4oc5ccccc5c4cc3-c3cccc4c3N(c3ccccc3)CN4c3ccccc3)cc2)cc1 ; CC1(C)c2ccccc2-c2cc(-c3cccc4c3N(c3ccccc3)CN4c3ccccc3)c(N(c3ccc(-c4ccccc4)cc3)c3ccc4ccc5ccccc5c4c3)cc21 ; CC1(C)c2ccccc2-c2c(-c3cccc4c3N(c3ccccc3)CN4c3ccccc3)cc(N(c3ccc(-c4ccccc4)cc3)c3ccc4ccc5cccc6ccc3c4c56)cc21 ; CC1(C)c2ccccc2-c2c(-c3cccc4c3N(c3ccccc3)CN4c3ccccc3)cc(N(c3ccc(-c4ccccc4)cc3)c3cccc(-c4nc5ccccc5o4)c3)cc21]

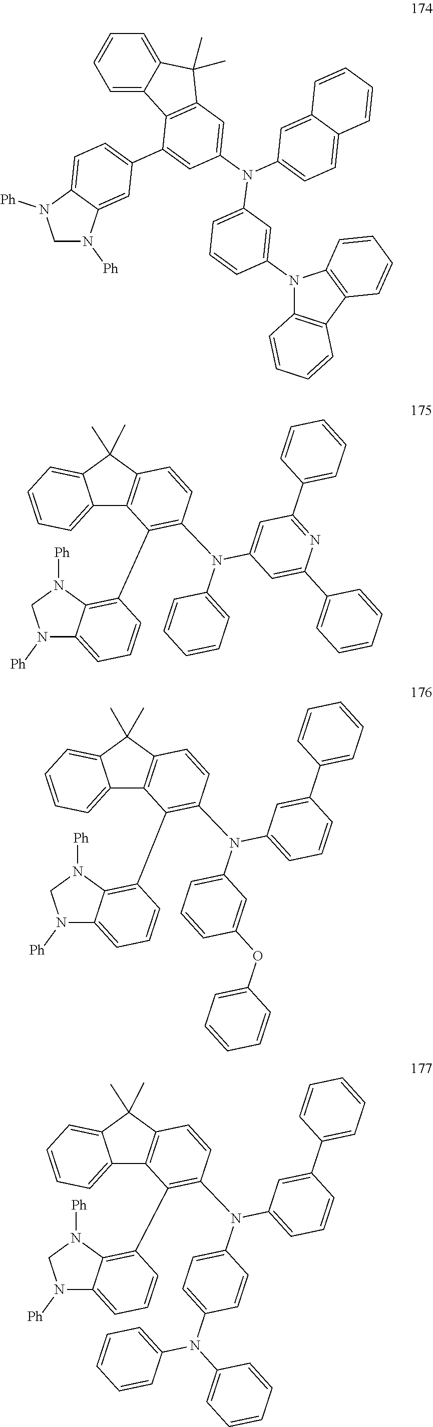

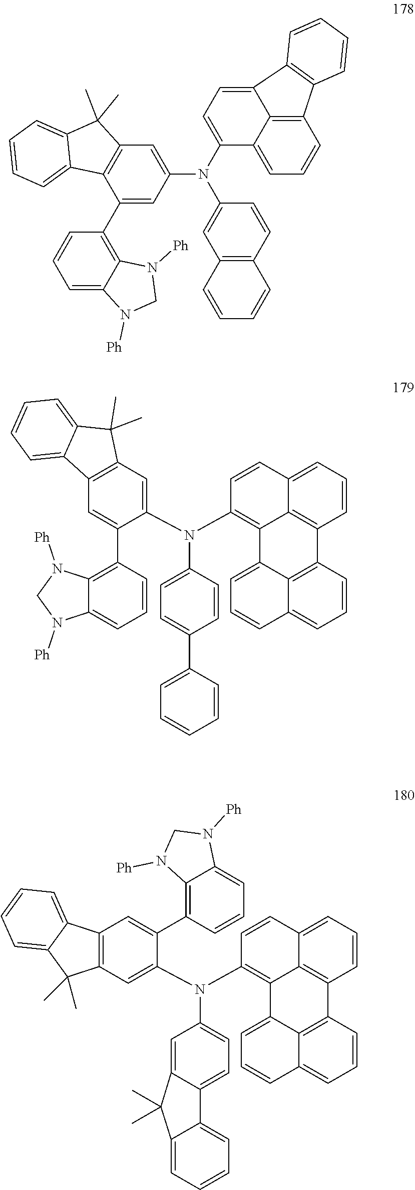 [SMILES: CC1(C)c2ccccc2-c2c(-c3cccc4c3N(c3ccccc3)CN4c3ccccc3)cc(N(c3ccc4ccccc4c3)c3ccc4c5c(cccc35)-c3ccccc3-4)cc21 ; CC1(C)c2ccccc2-c2cc(-c3cccc4c3N(c3ccccc3)CN4c3ccccc3)c(N(c3ccc(-c4ccccc4)cc3)c3ccc4cccc5c6cccc7cccc(c3c45)c76)cc21 ; CC1(C)c2ccccc2-c2ccc(N(c3cc4c(cc3-c3cccc5c3N(c3ccccc3)CN5c3ccccc3)-c3ccccc3C4(C)C)c3ccc4cccc5c6cccc7cccc(c3c45)c76)cc21]

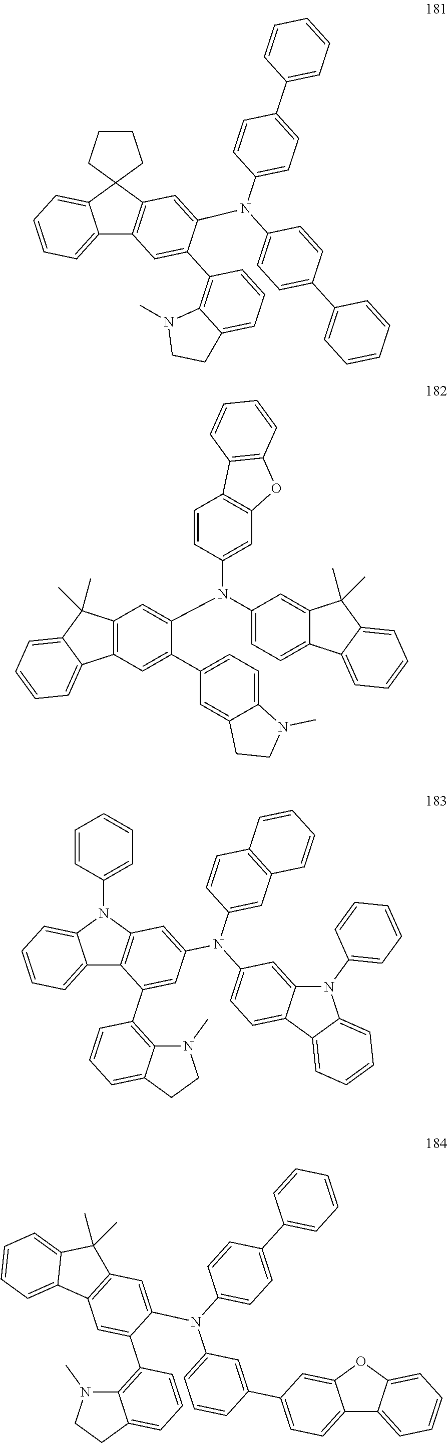

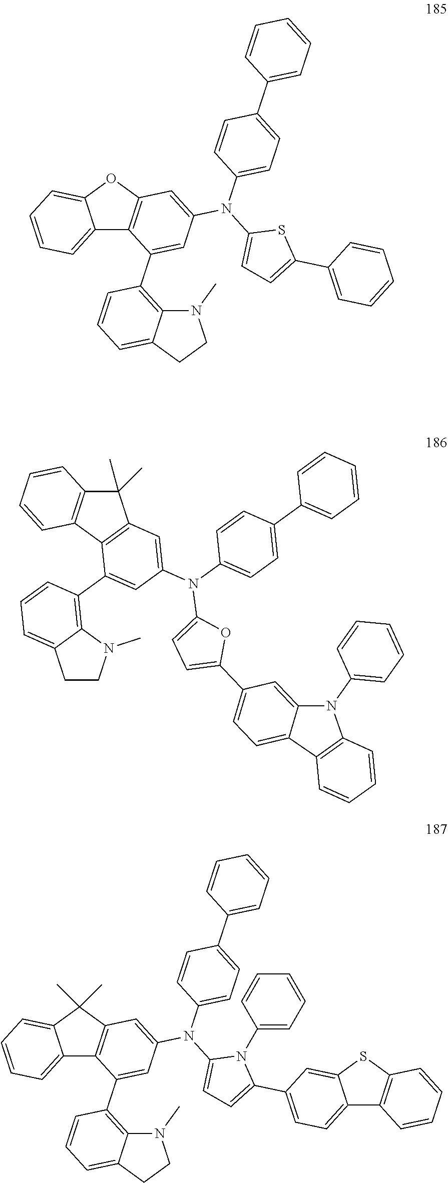 [SMILES: CN1CCc2cccc(-c3cc(N(c4ccc(-c5ccccc5)cc4)c4ccc(-c5ccccc5)s4)cc4oc5ccccc5c34)c21 ; CN1CCc2cccc(-c3cc(N(c4ccc(-c5ccccc5)cc4)c4ccc(-c5ccc6c7ccccc7n(-c7ccccc7)c6c5)o4)cc4c3-c3ccccc3C4(C)C)c21 ; CN1CCc2cccc(-c3cc(N(c4ccc(-c5ccccc5)cc4)c4ccc(-c5ccc6c(c5)sc5ccccc56)n4-c4ccccc4)cc4c3-c3ccccc3C4(C)C)c21]

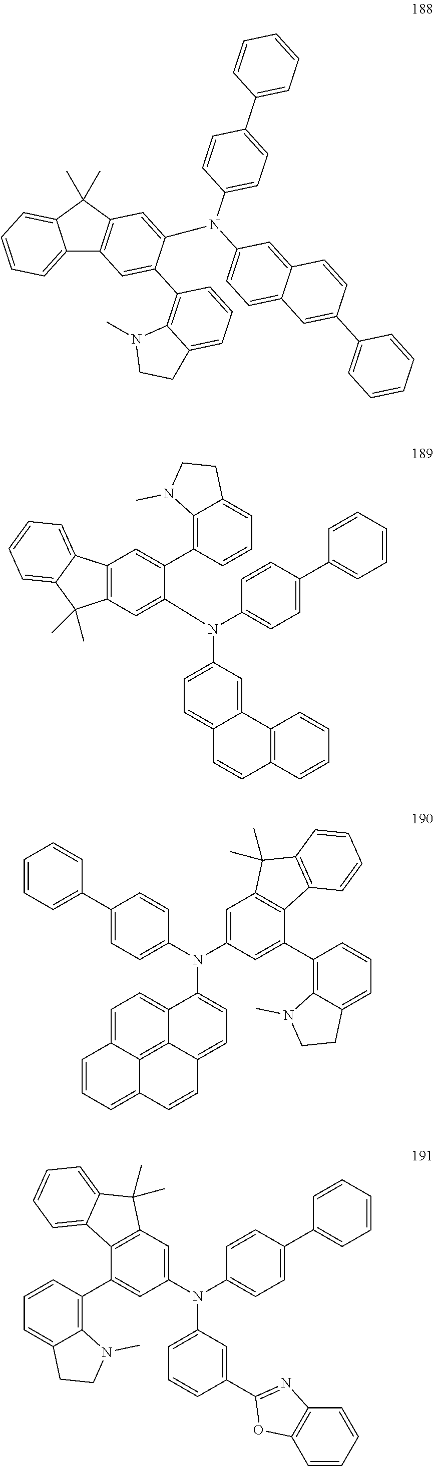 [SMILES: CN1CCc2cccc(-c3cc4c(cc3N(c3ccc(-c5ccccc5)cc3)c3ccc5cc(-c6ccccc6)ccc5c3)C(C)(C)c3ccccc3-4)c21 ; CN1CCc2cccc(-c3cc4c(cc3N(c3ccc(-c5ccccc5)cc3)c3ccc5ccc6ccccc6c5c3)C(C)(C)c3ccccc3-4)c21 ; CN1CCc2cccc(-c3cc(N(c4ccc(-c5ccccc5)cc4)c4ccc5ccc6cccc7ccc4c5c67)cc4c3-c3ccccc3C4(C)C)c21 ; CN1CCc2cccc(-c3cc(N(c4ccc(-c5ccccc5)cc4)c4cccc(-c5nc6ccccc6o5)c4)cc4c3-c3ccccc3C4(C)C)c21]

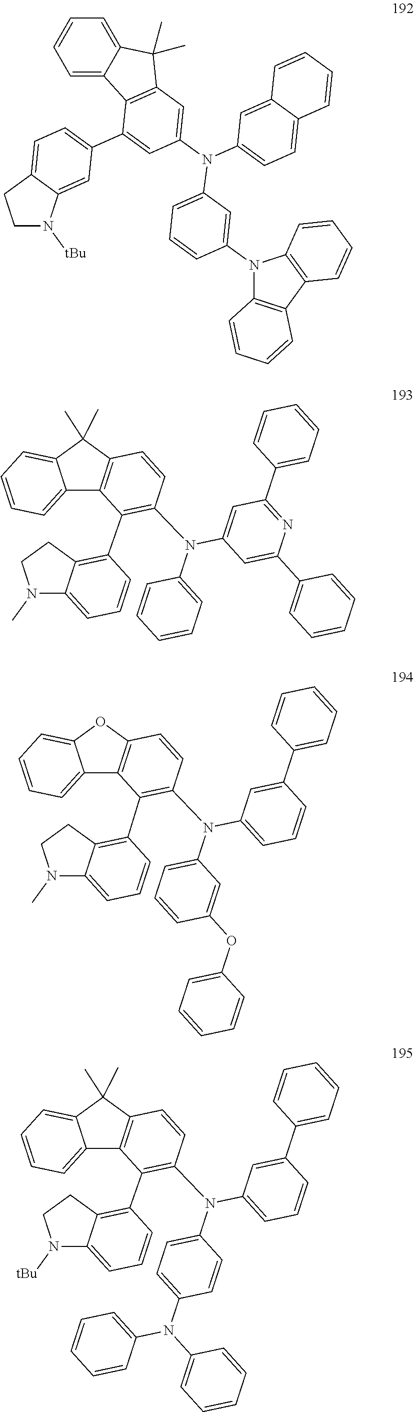 [SMILES: CC1(C)c2ccccc2-c2c(-c3ccc4c(c3)N(C(C)(C)C)CC4)cc(N(c3cccc(-n4c5ccccc5c5ccccc54)c3)c3ccc4ccccc4c3)cc21 ; CN1CCc2c(-c3c(N(c4ccccc4)c4cc(-c5ccccc5)nc(-c5ccccc5)c4)ccc4c3-c3ccccc3C4(C)C)cccc21 ; CN1CCc2c(-c3c(N(c4cccc(Oc5ccccc5)c4)c4cccc(-c5ccccc5)c4)ccc4oc5ccccc5c34)cccc21 ; CC1(C)c2ccccc2-c2c1ccc(N(c1ccc(N(c3ccccc3)c3ccccc3)cc1)c1cccc(-c3ccccc3)c1)c2-c1cccc2c1CCN2C(C)(C)C]

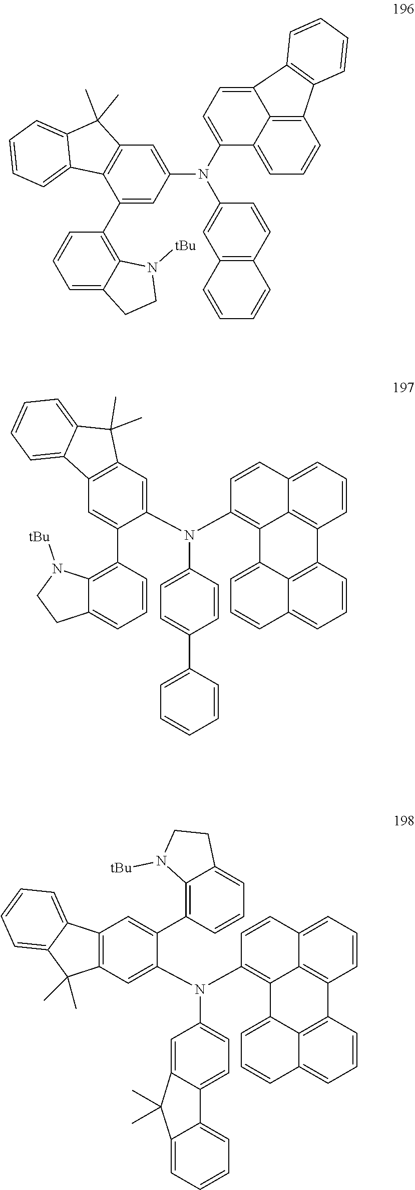 [SMILES: CC1(C)c2ccccc2-c2c(-c3cccc4c3N(C(C)(C)C)CC4)cc(N(c3ccc4ccccc4c3)c3ccc4c5c(cccc35)-c3ccccc3-4)cc21 ; CC1(C)c2ccccc2-c2cc(-c3cccc4c3N(C(C)(C)C)CC4)c(N(c3ccc(-c4ccccc4)cc3)c3ccc4cccc5c6cccc7cccc(c3c45)c76)cc21 ; CC1(C)c2ccccc2-c2ccc(N(c3cc4c(cc3-c3cccc5c3N(C(C)(C)C)CC5)-c3ccccc3C4(C)C)c3ccc4cccc5c6cccc7cccc(c3c45)c76)cc21]

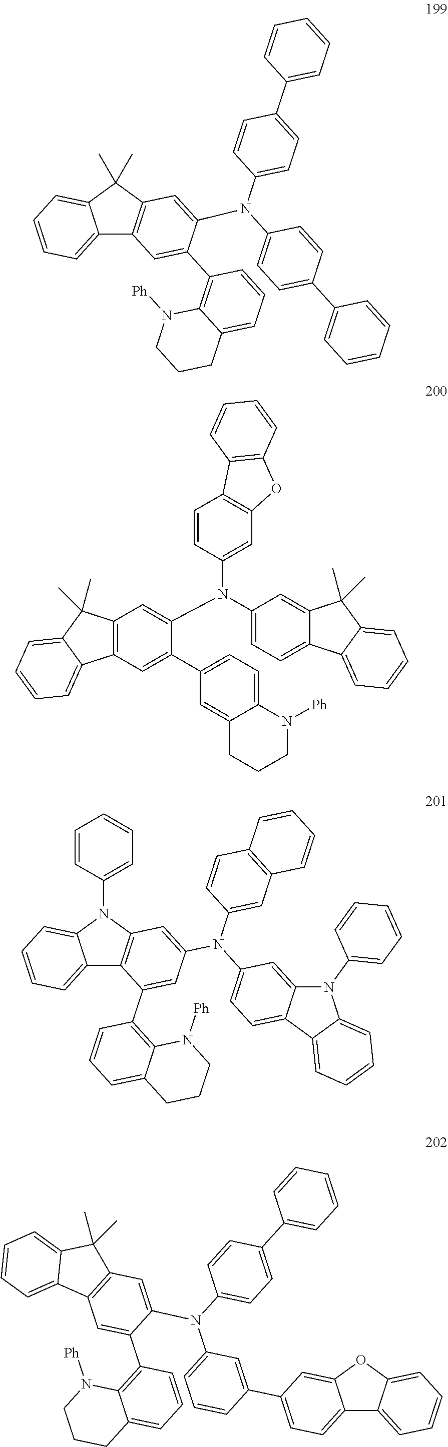

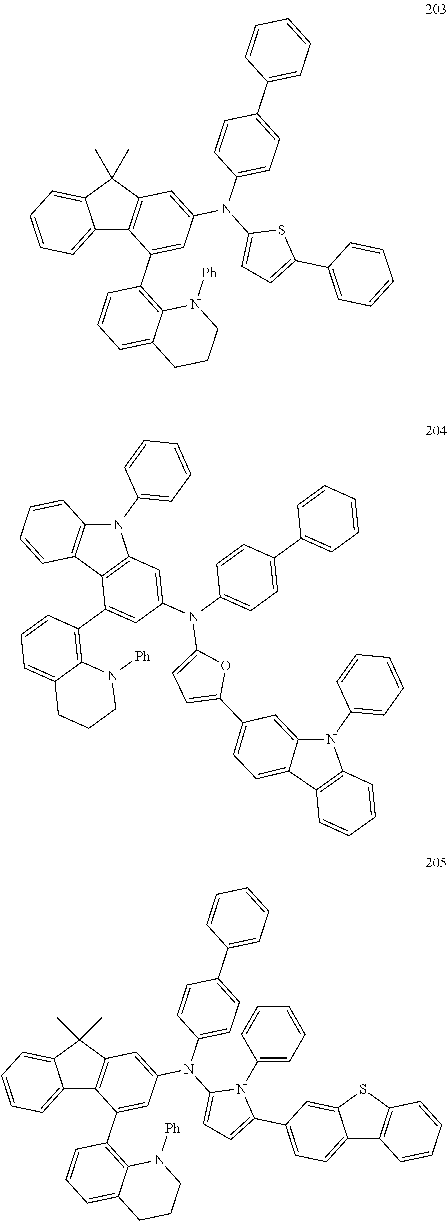 [SMILES: CC1(C)c2ccccc2-c2c(-c3cccc4c3N(c3ccccc3)CCC4)cc(N(c3ccc(-c4ccccc4)cc3)c3ccc(-c4ccccc4)s3)cc21 ; c1ccc(-c2ccc(N(c3cc(-c4cccc5c4N(c4ccccc4)CCC5)c4c5ccccc5n(-c5ccccc5)c4c3)c3ccc(-c4ccc5c6ccccc6n(-c6ccccc6)c5c4)o3)cc2)cc1 ; CC1(C)c2ccccc2-c2c(-c3cccc4c3N(c3ccccc3)CCC4)cc(N(c3ccc(-c4ccccc4)cc3)c3ccc(-c4ccc5c(c4)sc4ccccc45)n3-c3ccccc3)cc21]

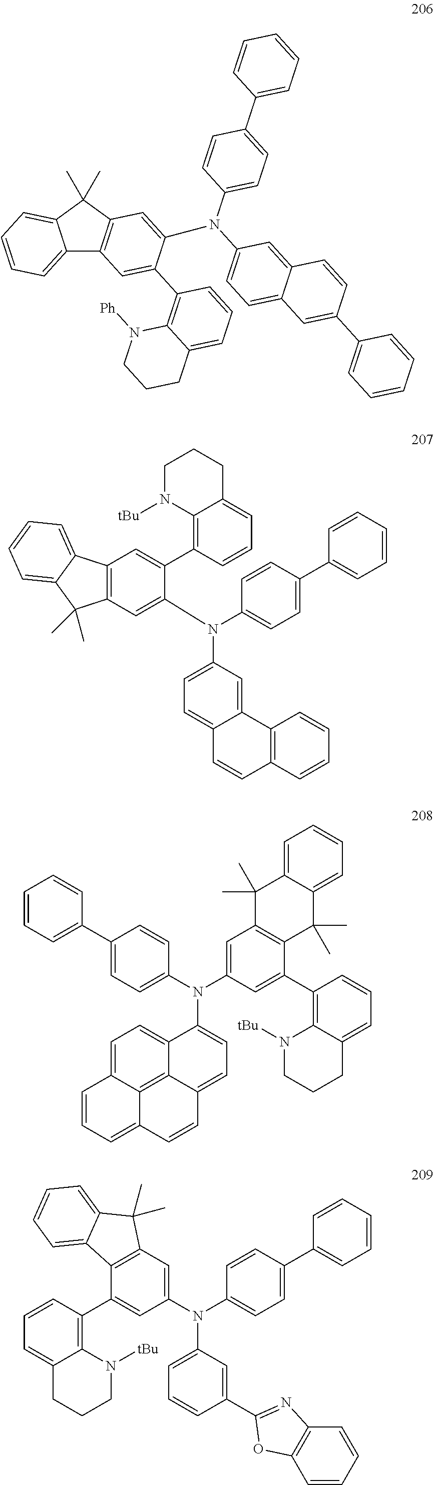 [SMILES: CC1(C)c2ccccc2-c2cc(-c3cccc4c3N(c3ccccc3)CCC4)c(N(c3ccc(-c4ccccc4)cc3)c3ccc4cc(-c5ccccc5)ccc4c3)cc21 ; CC1(C)c2ccccc2-c2cc(-c3cccc4c3N(C(C)(C)C)CCC4)c(N(c3ccc(-c4ccccc4)cc3)c3ccc4ccc5ccccc5c4c3)cc21 ; CC1(C)c2ccccc2C(C)(C)c2c(-c3cccc4c3N(C(C)(C)C)CCC4)cc(N(c3ccc(-c4ccccc4)cc3)c3ccc4ccc5cccc6ccc3c4c56)cc21 ; CC1(C)c2ccccc2-c2c(-c3cccc4c3N(C(C)(C)C)CCC4)cc(N(c3ccc(-c4ccccc4)cc3)c3cccc(-c4nc5ccccc5o4)c3)cc21]

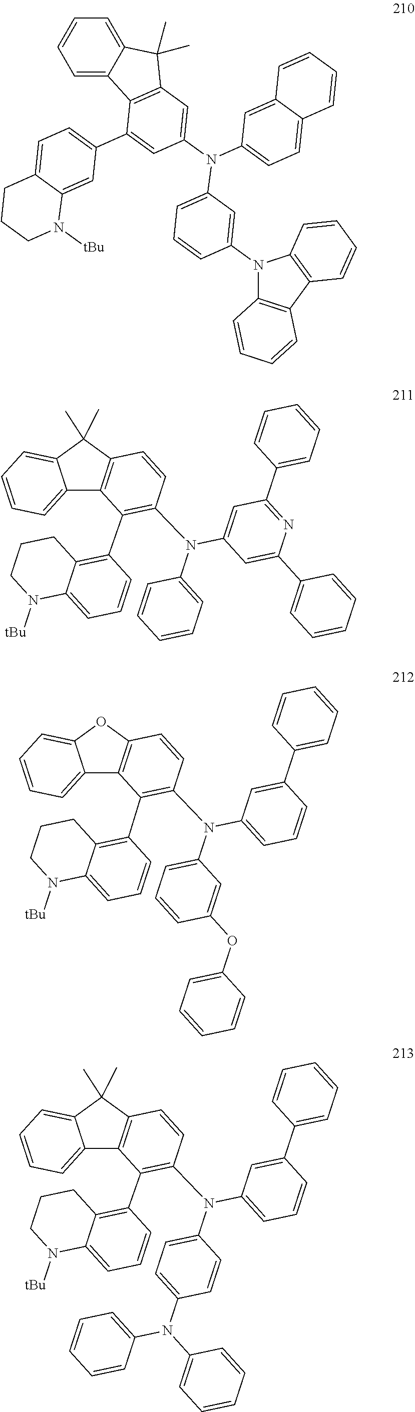 [SMILES: CC1(C)c2ccccc2-c2c(-c3ccc4c(c3)N(C(C)(C)C)CCC4)cc(N(c3cccc(-n4c5ccccc5c5ccccc54)c3)c3ccc4ccccc4c3)cc21 ; CC1(C)c2ccccc2-c2c1ccc(N(c1ccccc1)c1cc(-c3ccccc3)nc(-c3ccccc3)c1)c2-c1cccc2c1CCCN2C(C)(C)C ; CC(C)(C)N1CCCc2c(-c3c(N(c4cccc(Oc5ccccc5)c4)c4cccc(-c5ccccc5)c4)ccc4oc5ccccc5c34)cccc21 ; CC1(C)c2ccccc2-c2c1ccc(N(c1ccc(N(c3ccccc3)c3ccccc3)cc1)c1cccc(-c3ccccc3)c1)c2-c1cccc2c1CCCN2C(C)(C)C]

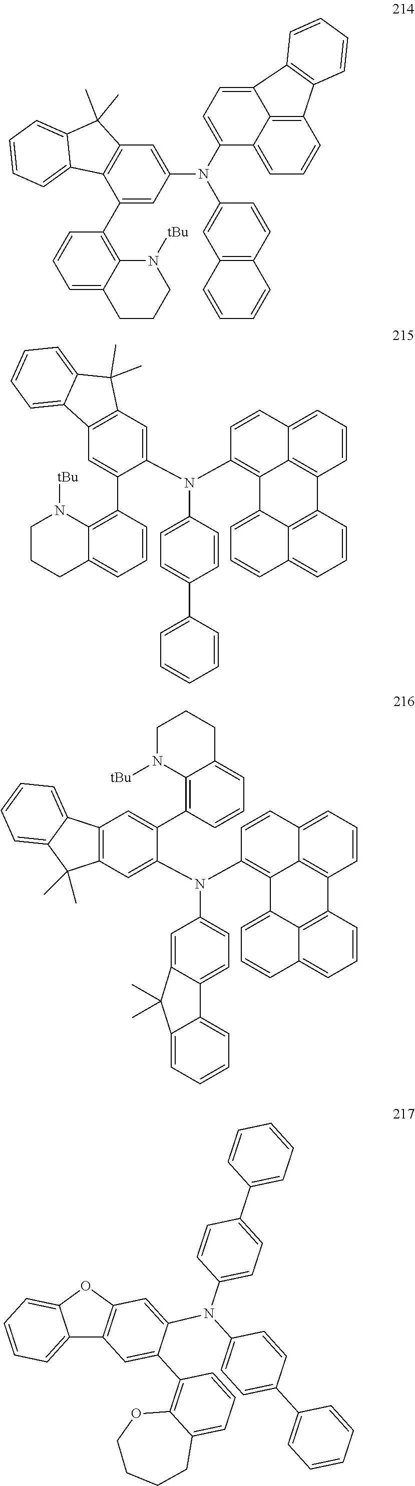

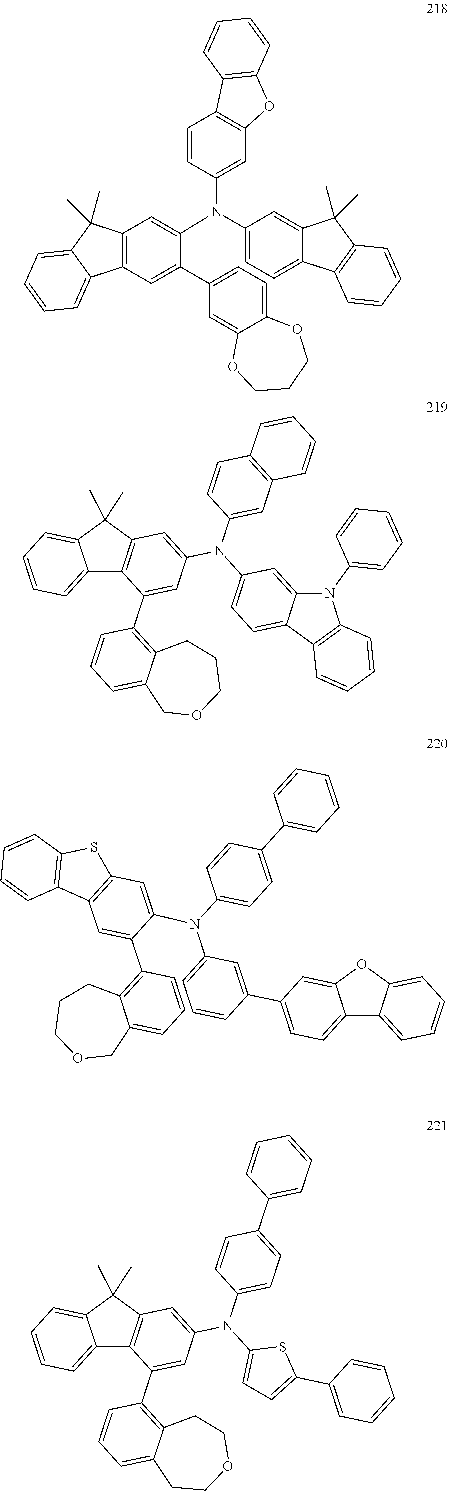

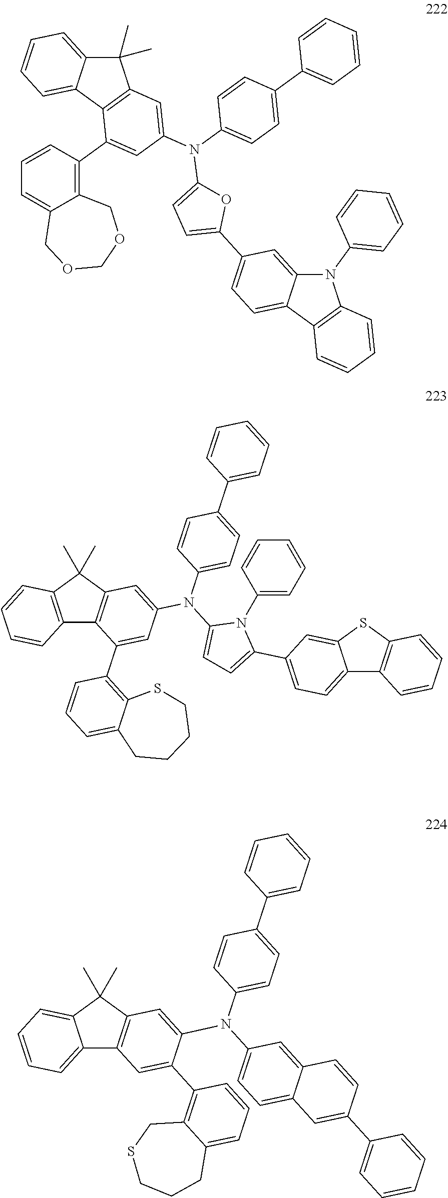 [SMILES: CC1(C)c2ccccc2-c2c(-c3cccc4c3COCOC4)cc(N(c3ccc(-c4ccccc4)cc3)c3ccc(-c4ccc5c6ccccc6n(-c6ccccc6)c5c4)o3)cc21 ; CC1(C)c2ccccc2-c2c(-c3cccc4c3SCCCC4)cc(N(c3ccc(-c4ccccc4)cc3)c3ccc(-c4ccc5c(c4)sc4ccccc45)n3-c3ccccc3)cc21 ; CC1(C)c2ccccc2-c2cc(-c3cccc4c3CSCCC4)c(N(c3ccc(-c4ccccc4)cc3)c3ccc4cc(-c5ccccc5)ccc4c3)cc21]

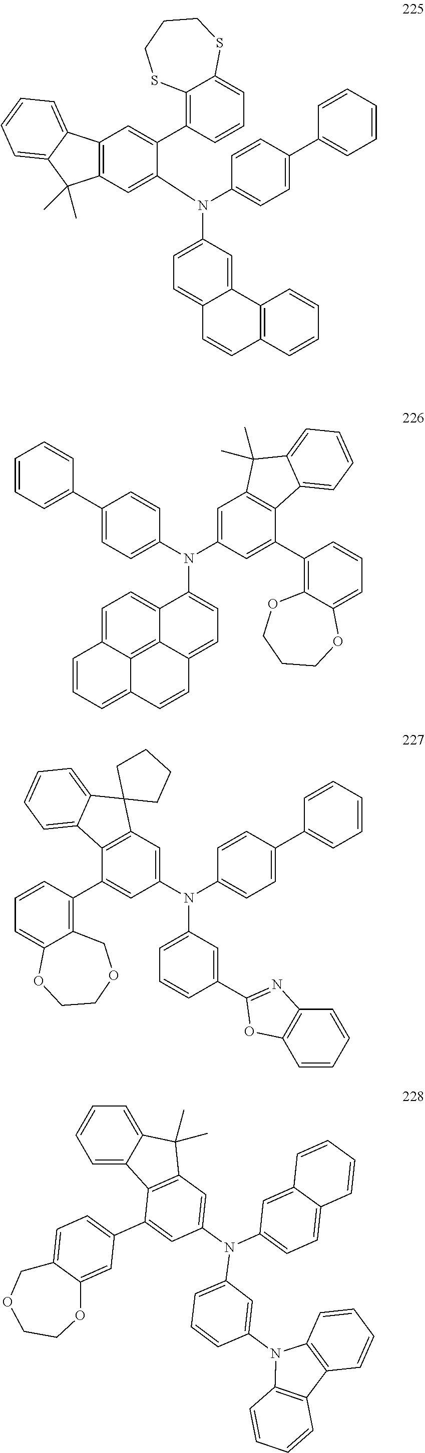

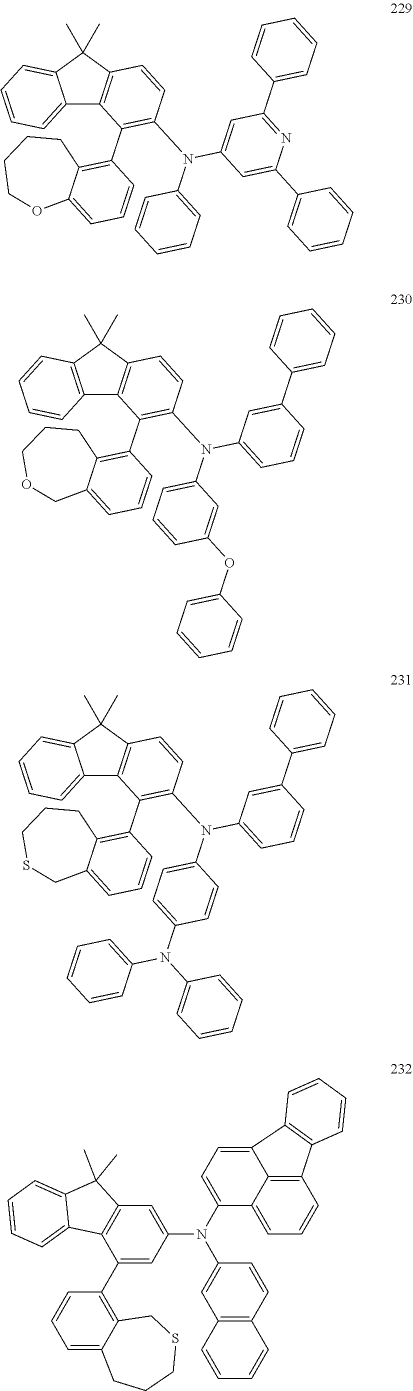 [SMILES: CC1(C)c2ccccc2-c2c1ccc(N(c1ccccc1)c1cc(-c3ccccc3)nc(-c3ccccc3)c1)c2-c1cccc2c1CCCCO2 ; CC1(C)c2ccccc2-c2c1ccc(N(c1cccc(Oc3ccccc3)c1)c1cccc(-c3ccccc3)c1)c2-c1cccc2c1CCCOC2 ; CC1(C)c2ccccc2-c2c1ccc(N(c1ccc(N(c3ccccc3)c3ccccc3)cc1)c1cccc(-c3ccccc3)c1)c2-c1cccc2c1CCCSC2 ; CC1(C)c2ccccc2-c2c(-c3cccc4c3CSCCC4)cc(N(c3ccc4ccccc4c3)c3ccc4c5c(cccc35)-c3ccccc3-4)cc21]

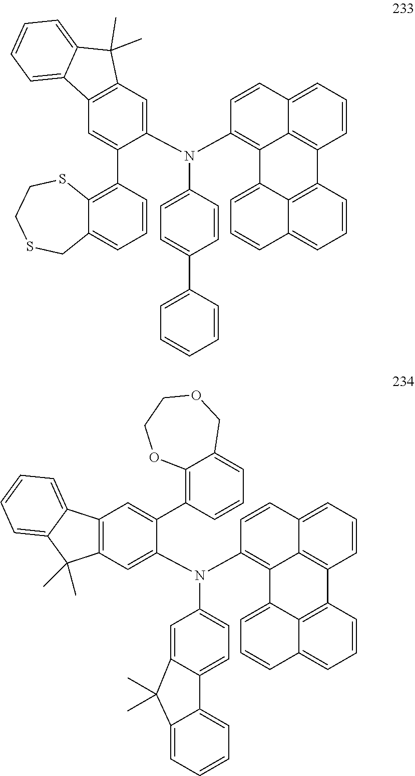

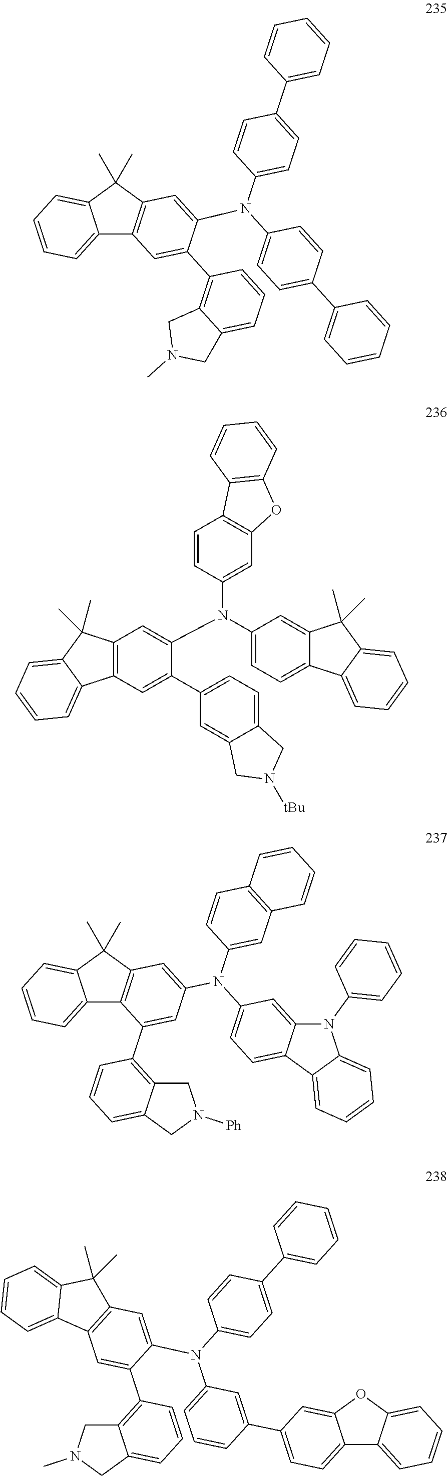

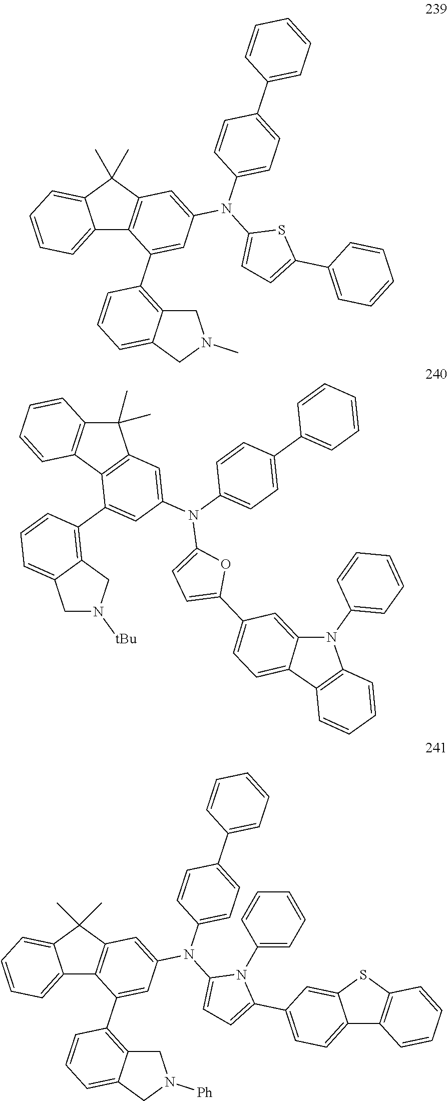

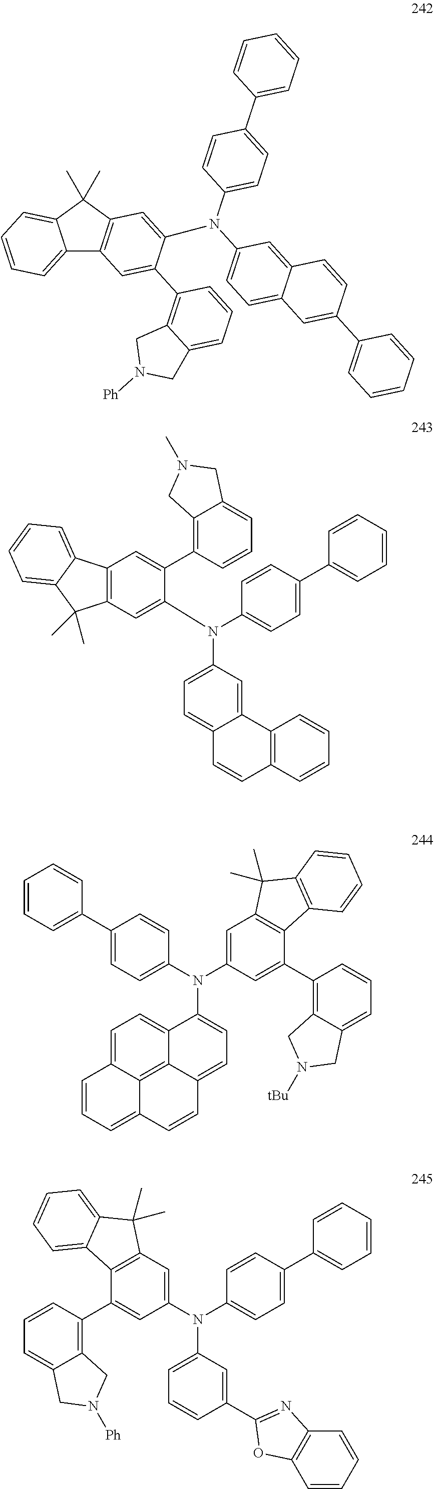 [SMILES: CC1(C)c2ccccc2-c2cc(-c3cccc4c3CN(c3ccccc3)C4)c(N(c3ccc(-c4ccccc4)cc3)c3ccc4cc(-c5ccccc5)ccc4c3)cc21 ; CN1Cc2cccc(-c3cc4c(cc3N(c3ccc(-c5ccccc5)cc3)c3ccc5ccc6ccccc6c5c3)C(C)(C)c3ccccc3-4)c2C1 ; CC1(C)c2ccccc2-c2c(-c3cccc4c3CN(C(C)(C)C)C4)cc(N(c3ccc(-c4ccccc4)cc3)c3ccc4ccc5cccc6ccc3c4c56)cc21 ; CC1(C)c2ccccc2-c2c(-c3cccc4c3CN(c3ccccc3)C4)cc(N(c3ccc(-c4ccccc4)cc3)c3cccc(-c4nc5ccccc5o4)c3)cc21]

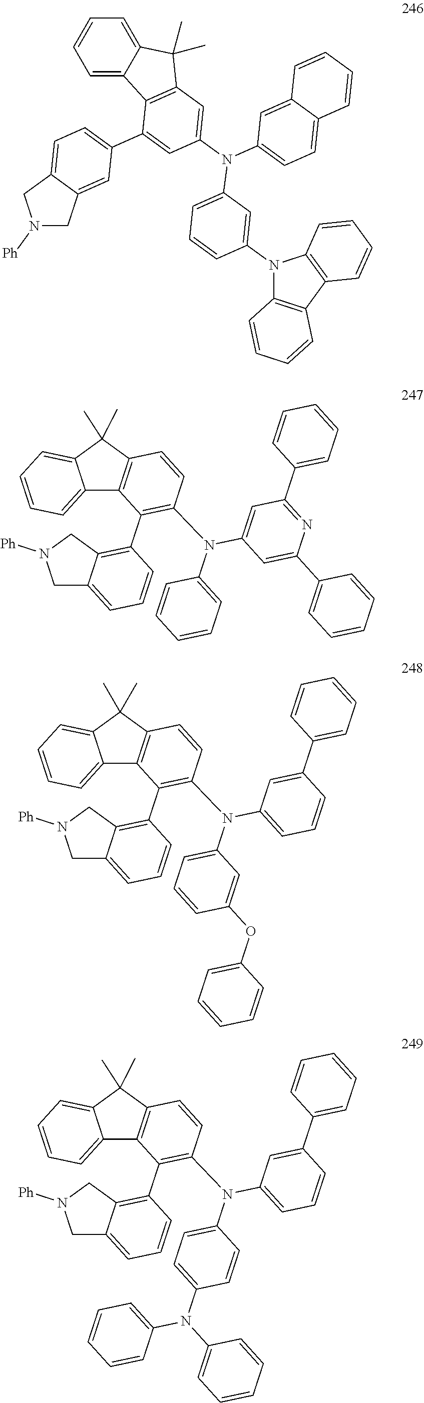

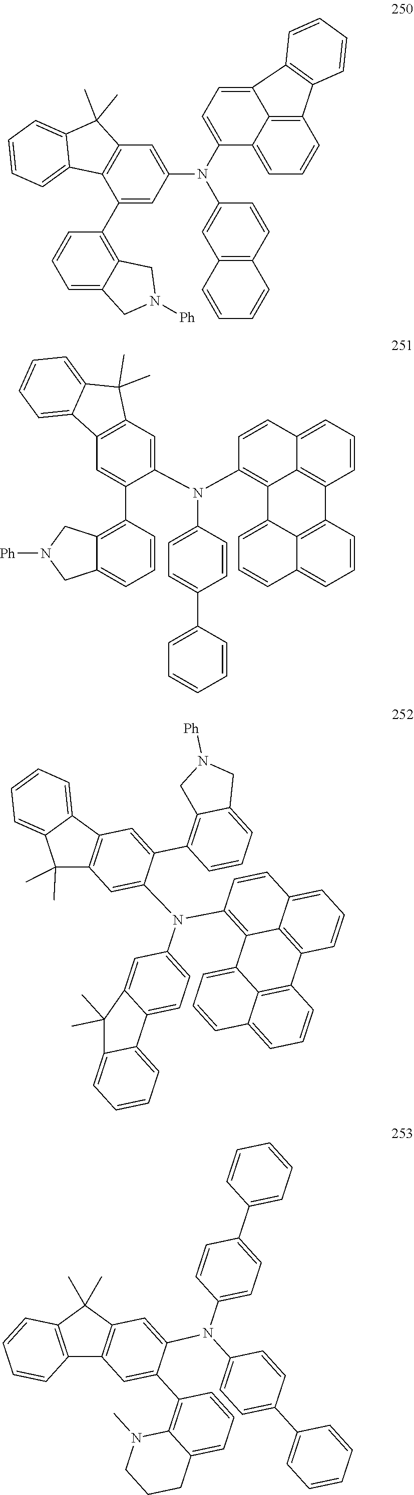

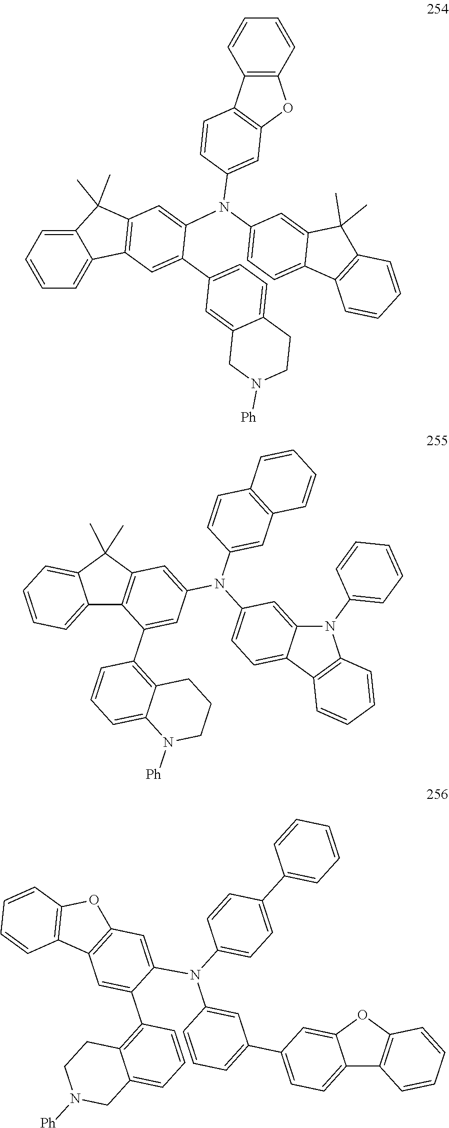

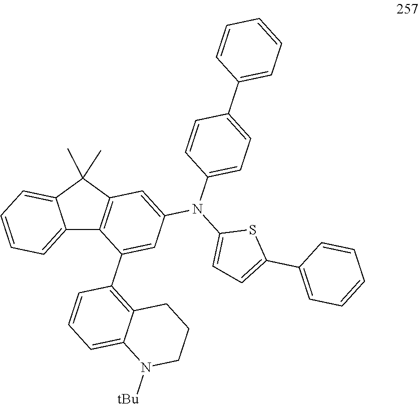 [SMILES: CC1(C)c2ccccc2-c2c(-c3cccc4c3CCCN4C(C)(C)C)cc(N(c3ccc(-c4ccccc4)cc3)c3ccc(-c4ccccc4)s3)cc21]

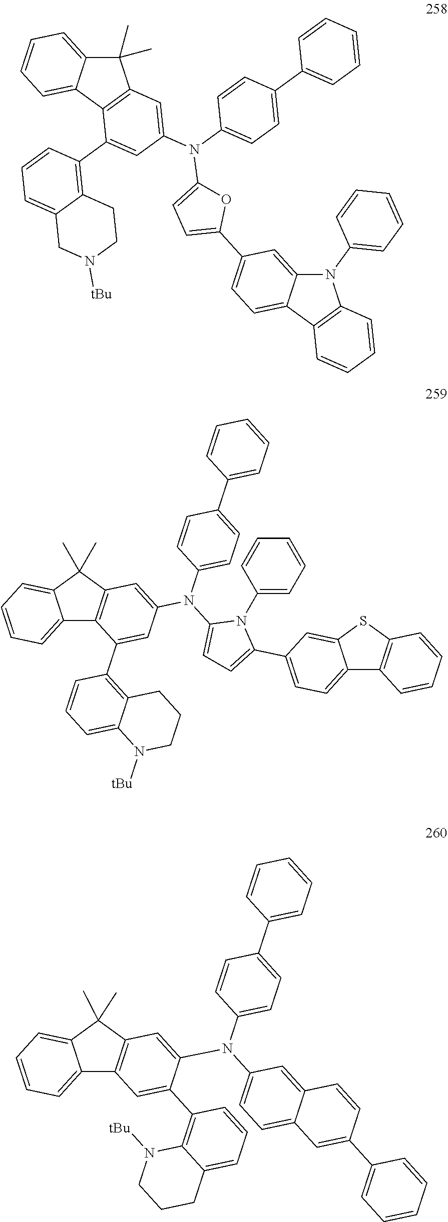 [SMILES: CC1(C)c2ccccc2-c2c(-c3cccc4c3CCN(C(C)(C)C)C4)cc(N(c3ccc(-c4ccccc4)cc3)c3ccc(-c4ccc5c6ccccc6n(-c6ccccc6)c5c4)o3)cc21 ; CC1(C)c2ccccc2-c2c(-c3cccc4c3CCCN4C(C)(C)C)cc(N(c3ccc(-c4ccccc4)cc3)c3ccc(-c4ccc5c(c4)sc4ccccc45)n3-c3ccccc3)cc21 ; CC1(C)c2ccccc2-c2cc(-c3cccc4c3N(C(C)(C)C)CCC4)c(N(c3ccc(-c4ccccc4)cc3)c3ccc4cc(-c5ccccc5)ccc4c3)cc21]

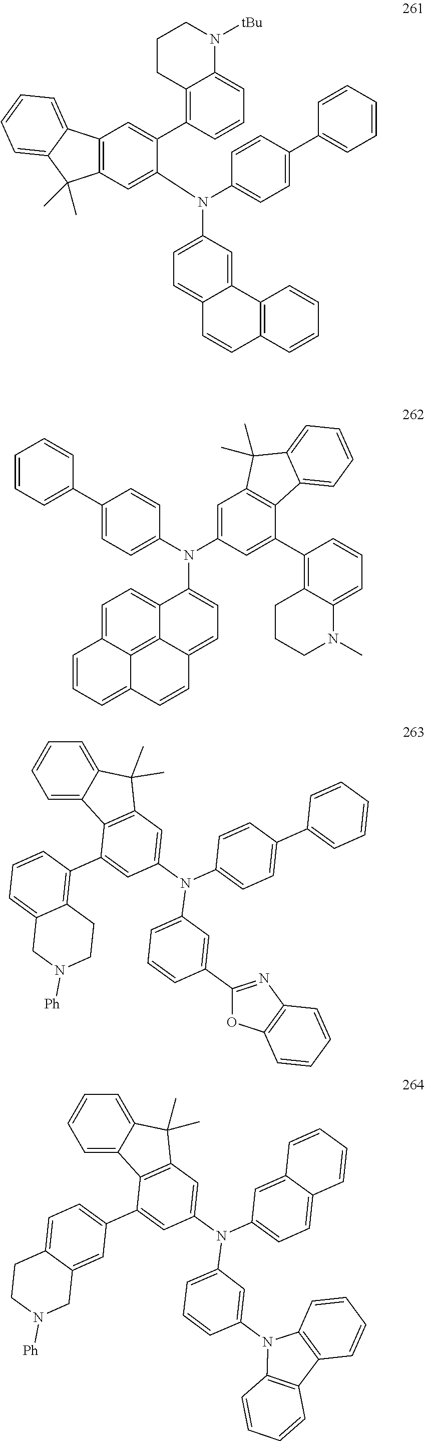

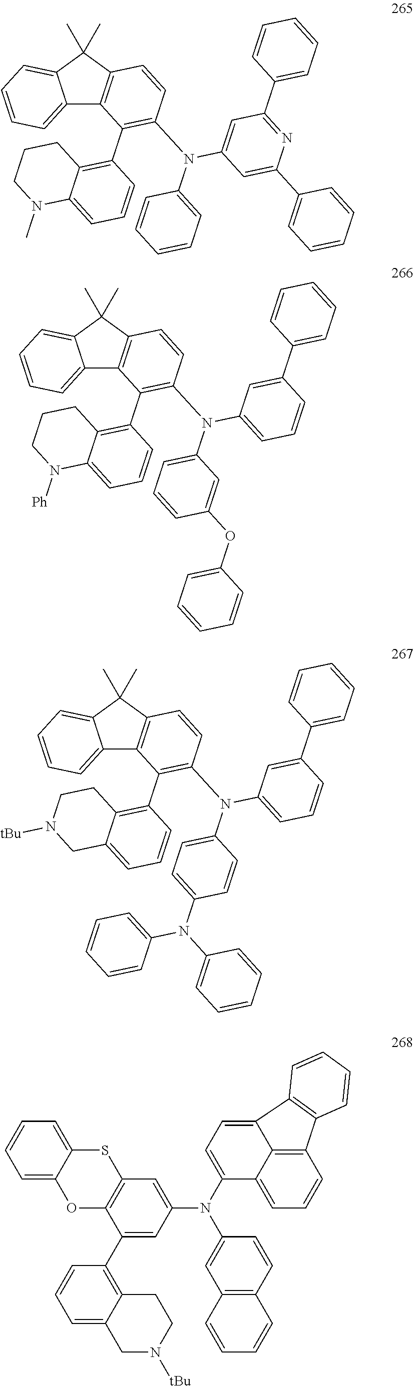

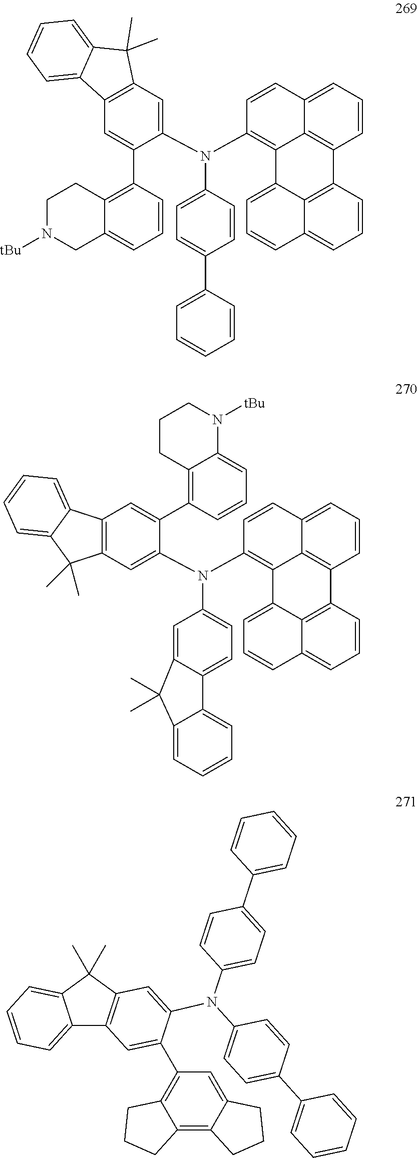 [SMILES: CC1(C)c2ccccc2-c2cc(-c3cccc4c3CCN(C(C)(C)C)C4)c(N(c3ccc(-c4ccccc4)cc3)c3ccc4cccc5c6cccc7cccc(c3c45)c76)cc21 ; CC1(C)c2ccccc2-c2ccc(N(c3cc4c(cc3-c3cccc5c3CCCN5C(C)(C)C)-c3ccccc3C4(C)C)c3ccc4cccc5c6cccc7cccc(c3c45)c76)cc21 ; CC1(C)c2ccccc2-c2cc(-c3cc4c(c5c3CCC5)CCC4)c(N(c3ccc(-c4ccccc4)cc3)c3ccc(-c4ccccc4)cc3)cc21]

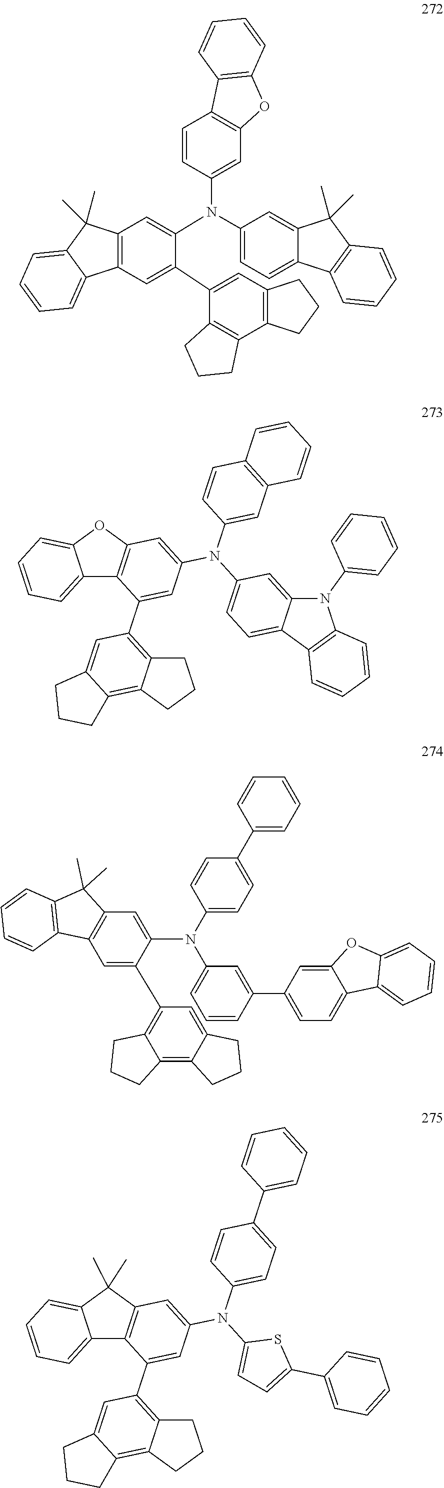 [SMILES: CC1(C)c2ccccc2-c2ccc(N(c3ccc4c(c3)oc3ccccc34)c3cc4c(cc3-c3cc5c(c6c3CCC6)CCC5)-c3ccccc3C4(C)C)cc21 ; c1ccc(-n2c3ccccc3c3ccc(N(c4ccc5ccccc5c4)c4cc(-c5cc6c(c7c5CCC7)CCC6)c5c(c4)oc4ccccc45)cc32)cc1 ; CC1(C)c2ccccc2-c2cc(-c3cc4c(c5c3CCC5)CCC4)c(N(c3ccc(-c4ccccc4)cc3)c3cccc(-c4ccc5c(c4)oc4ccccc45)c3)cc21 ; CC1(C)c2ccccc2-c2c(-c3cc4c(c5c3CCC5)CCC4)cc(N(c3ccc(-c4ccccc4)cc3)c3ccc(-c4ccccc4)s3)cc21]

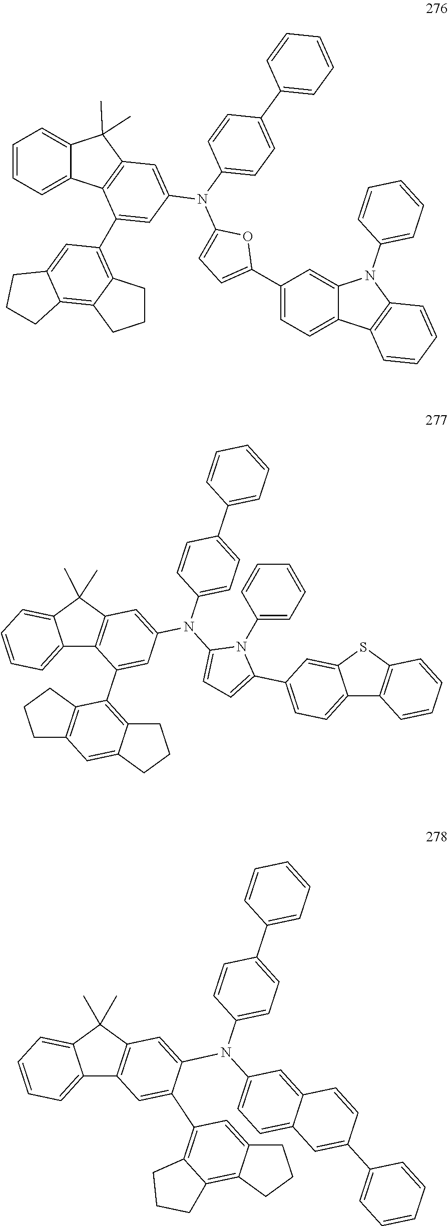 [SMILES: CC1(C)c2ccccc2-c2c(-c3cc4c(c5c3CCC5)CCC4)cc(N(c3ccc(-c4ccccc4)cc3)c3ccc(-c4ccc5c6ccccc6n(-c6ccccc6)c5c4)o3)cc21 ; CC1(C)c2ccccc2-c2c(-c3c4c(cc5c3CCC5)CCC4)cc(N(c3ccc(-c4ccccc4)cc3)c3ccc(-c4ccc5c(c4)sc4ccccc45)n3-c3ccccc3)cc21 ; CC1(C)c2ccccc2-c2cc(-c3cc4c(c5c3CCC5)CCC4)c(N(c3ccc(-c4ccccc4)cc3)c3ccc4cc(-c5ccccc5)ccc4c3)cc21]

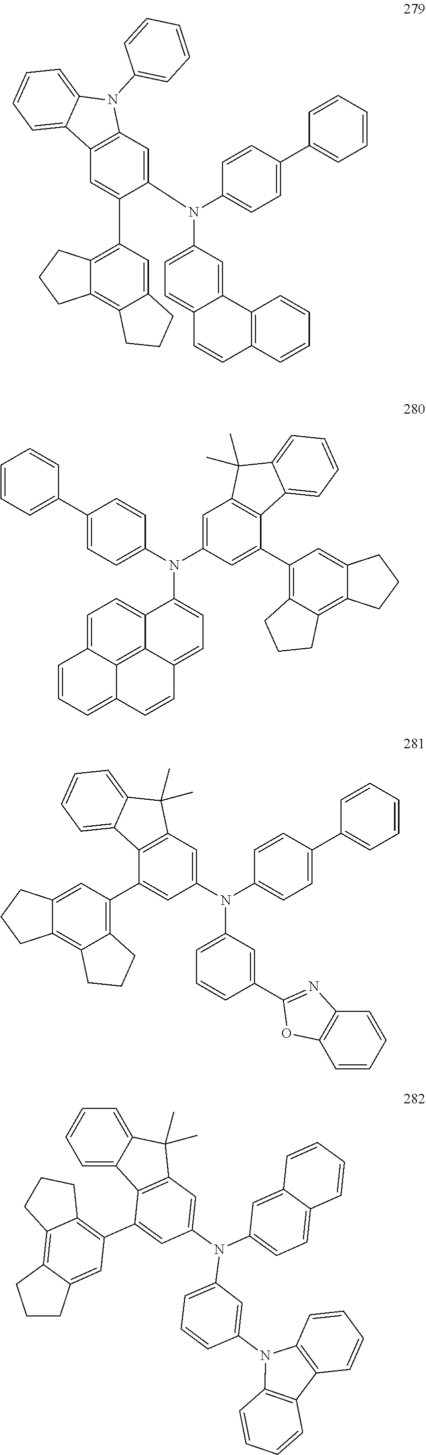 [SMILES: c1ccc(-c2ccc(N(c3ccc4ccc5ccccc5c4c3)c3cc4c(cc3-c3cc5c(c6c3CCC6)CCC5)c3ccccc3n4-c3ccccc3)cc2)cc1 ; CC1(C)c2ccccc2-c2c(-c3cc4c(c5c3CCC5)CCC4)cc(N(c3ccc(-c4ccccc4)cc3)c3ccc4ccc5cccc6ccc3c4c56)cc21 ; CC1(C)c2ccccc2-c2c(-c3cc4c(c5c3CCC5)CCC4)cc(N(c3ccc(-c4ccccc4)cc3)c3cccc(-c4nc5ccccc5o4)c3)cc21 ; CC1(C)c2ccccc2-c2c(-c3cc4c(c5c3CCC5)CCC4)cc(N(c3cccc(-n4c5ccccc5c5ccccc54)c3)c3ccc4ccccc4c3)cc21]

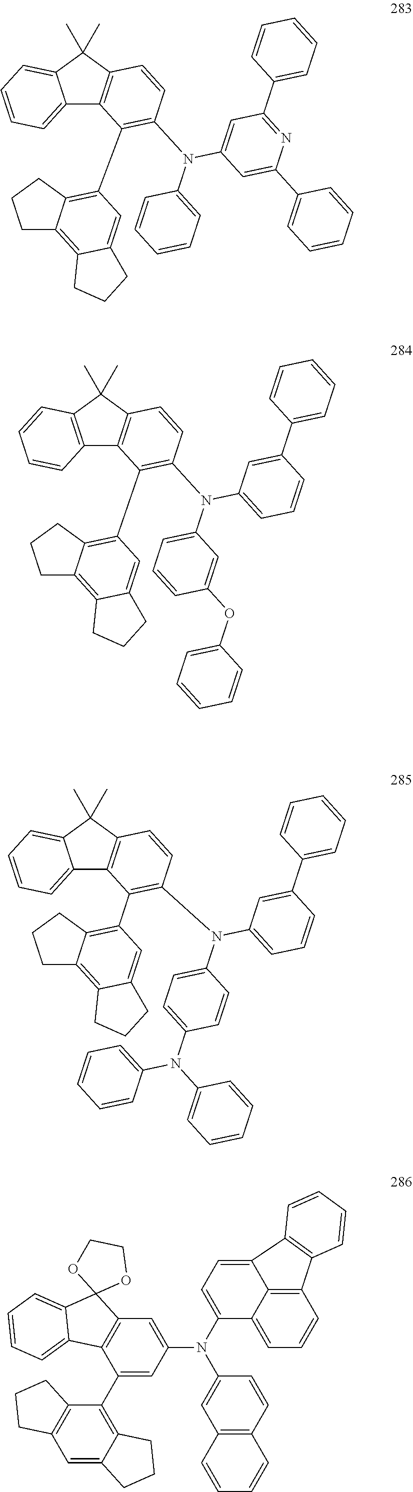 [SMILES: CC1(C)c2ccccc2-c2c1ccc(N(c1ccccc1)c1cc(-c3ccccc3)nc(-c3ccccc3)c1)c2-c1cc2c(c3c1CCC3)CCC2 ; CC1(C)c2ccccc2-c2c1ccc(N(c1cccc(Oc3ccccc3)c1)c1cccc(-c3ccccc3)c1)c2-c1cc2c(c3c1CCC3)CCC2 ; CC1(C)c2ccccc2-c2c1ccc(N(c1ccc(N(c3ccccc3)c3ccccc3)cc1)c1cccc(-c3ccccc3)c1)c2-c1cc2c(c3c1CCC3)CCC2 ; c1ccc2c(c1)-c1cccc3c(N(c4cc(-c5c6c(cc7c5CCC7)CCC6)c5c(c4)C4(OCCO4)c4ccccc4-5)c4ccc5ccccc5c4)ccc-2c13]

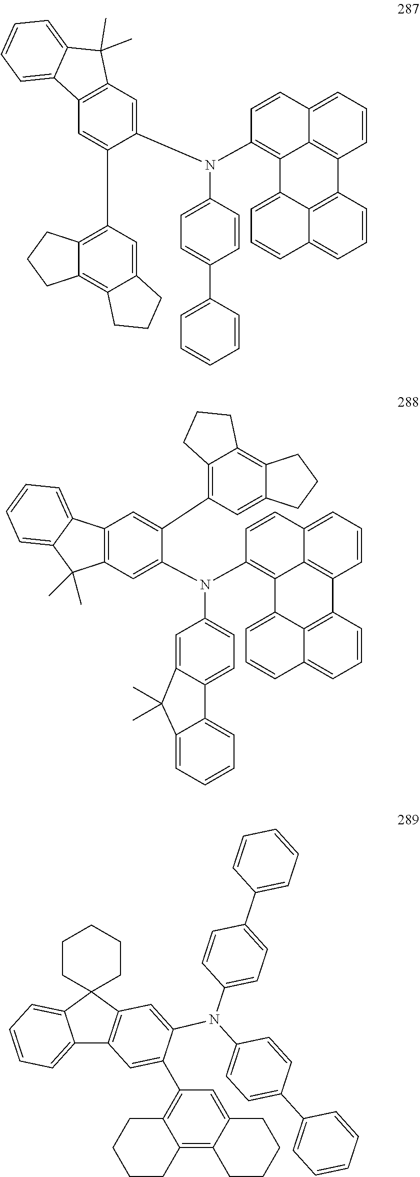 [SMILES: CC1(C)c2ccccc2-c2cc(-c3cc4c(c5c3CCC5)CCC4)c(N(c3ccc(-c4ccccc4)cc3)c3ccc4cccc5c6cccc7cccc(c3c45)c76)cc21 ; CC1(C)c2ccccc2-c2ccc(N(c3cc4c(cc3-c3cc5c(c6c3CCC6)CCC5)-c3ccccc3C4(C)C)c3ccc4cccc5c6cccc7cccc(c3c45)c76)cc21 ; c1ccc(-c2ccc(N(c3ccc(-c4ccccc4)cc3)c3cc4c(cc3-c3cc5c(c6c3CCCC6)CCCC5)-c3ccccc3C43CCCCC3)cc2)cc1]

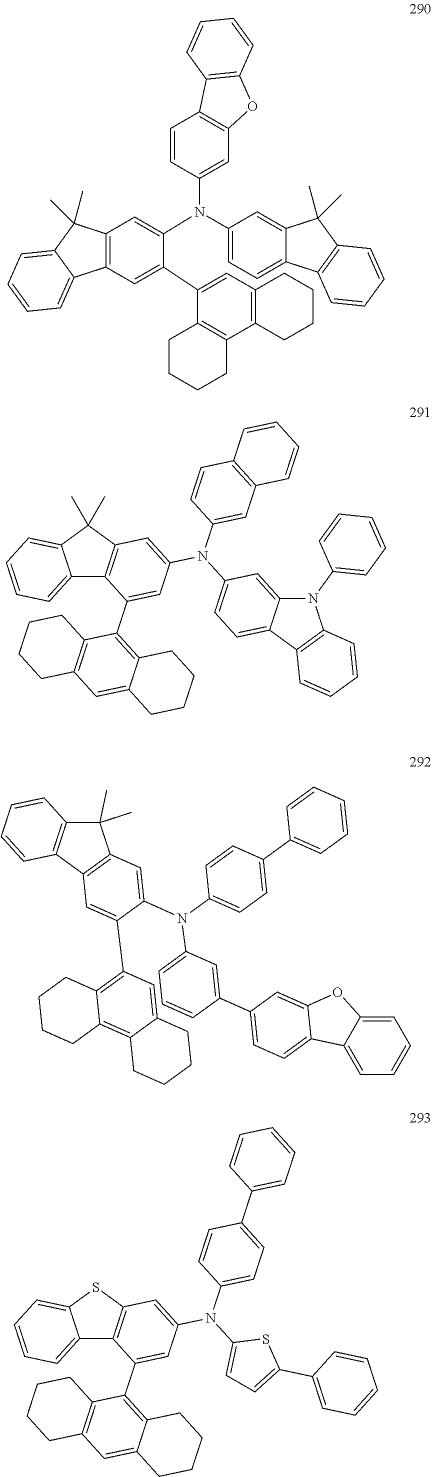 [SMILES: CC1(C)c2ccccc2-c2ccc(N(c3ccc4c(c3)oc3ccccc34)c3cc4c(cc3-c3cc5c(c6c3CCCC6)CCCC5)-c3ccccc3C4(C)C)cc21 ; CC1(C)c2ccccc2-c2c(-c3c4c(cc5c3CCCC5)CCCC4)cc(N(c3ccc4ccccc4c3)c3ccc4c5ccccc5n(-c5ccccc5)c4c3)cc21 ; CC1(C)c2ccccc2-c2cc(-c3cc4c(c5c3CCCC5)CCCC4)c(N(c3ccc(-c4ccccc4)cc3)c3cccc(-c4ccc5c(c4)oc4ccccc45)c3)cc21 ; c1ccc(-c2ccc(N(c3cc(-c4c5c(cc6c4CCCC6)CCCC5)c4c(c3)sc3ccccc34)c3ccc(-c4ccccc4)s3)cc2)cc1]

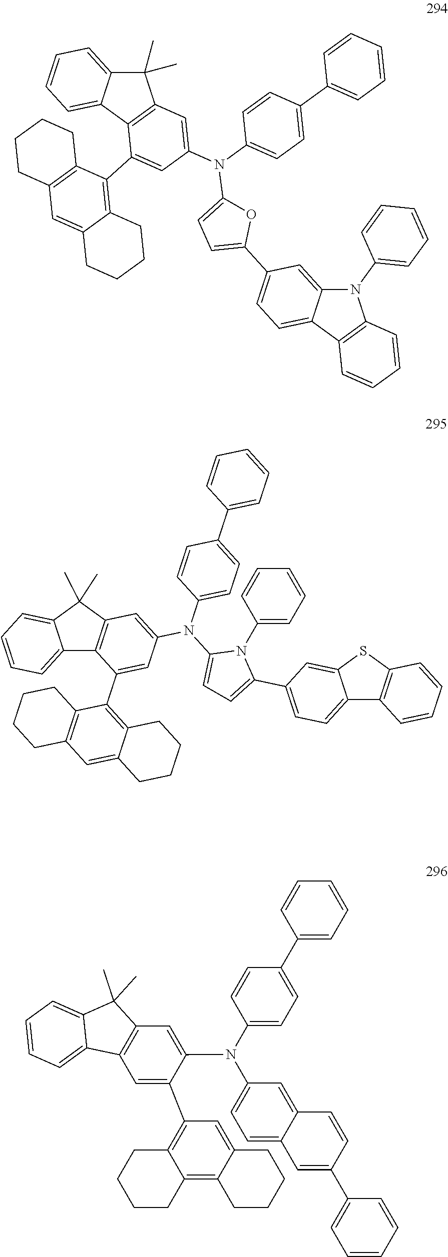 [SMILES: CC1(C)c2ccccc2-c2c(-c3c4c(cc5c3CCCC5)CCCC4)cc(N(c3ccc(-c4ccccc4)cc3)c3ccc(-c4ccc5c6ccccc6n(-c6ccccc6)c5c4)o3)cc21 ; CC1(C)c2ccccc2-c2c(-c3c4c(cc5c3CCCC5)CCCC4)cc(N(c3ccc(-c4ccccc4)cc3)c3ccc(-c4ccc5c(c4)sc4ccccc45)n3-c3ccccc3)cc21 ; CC1(C)c2ccccc2-c2cc(-c3cc4c(c5c3CCCC5)CCCC4)c(N(c3ccc(-c4ccccc4)cc3)c3ccc4cc(-c5ccccc5)ccc4c3)cc21]

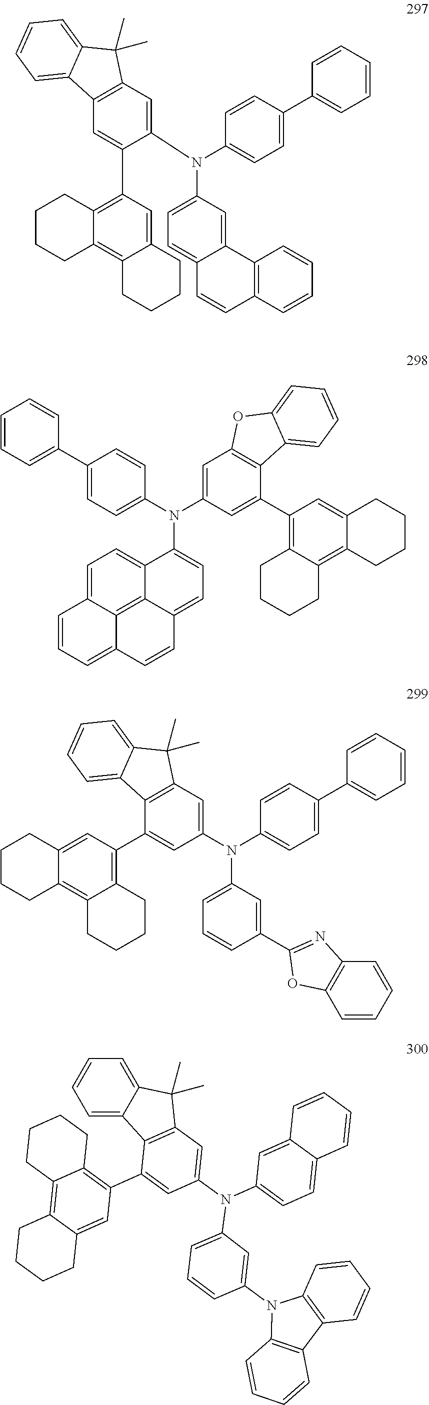 [SMILES: CC1(C)c2ccccc2-c2cc(-c3cc4c(c5c3CCCC5)CCCC4)c(N(c3ccc(-c4ccccc4)cc3)c3ccc4ccc5ccccc5c4c3)cc21 ; c1ccc(-c2ccc(N(c3cc(-c4cc5c(c6c4CCCC6)CCCC5)c4c(c3)oc3ccccc34)c3ccc4ccc5cccc6ccc3c4c56)cc2)cc1 ; CC1(C)c2ccccc2-c2c(-c3cc4c(c5c3CCCC5)CCCC4)cc(N(c3ccc(-c4ccccc4)cc3)c3cccc(-c4nc5ccccc5o4)c3)cc21 ; CC1(C)c2ccccc2-c2c(-c3cc4c(c5c3CCCC5)CCCC4)cc(N(c3cccc(-n4c5ccccc5c5ccccc54)c3)c3ccc4ccccc4c3)cc21]

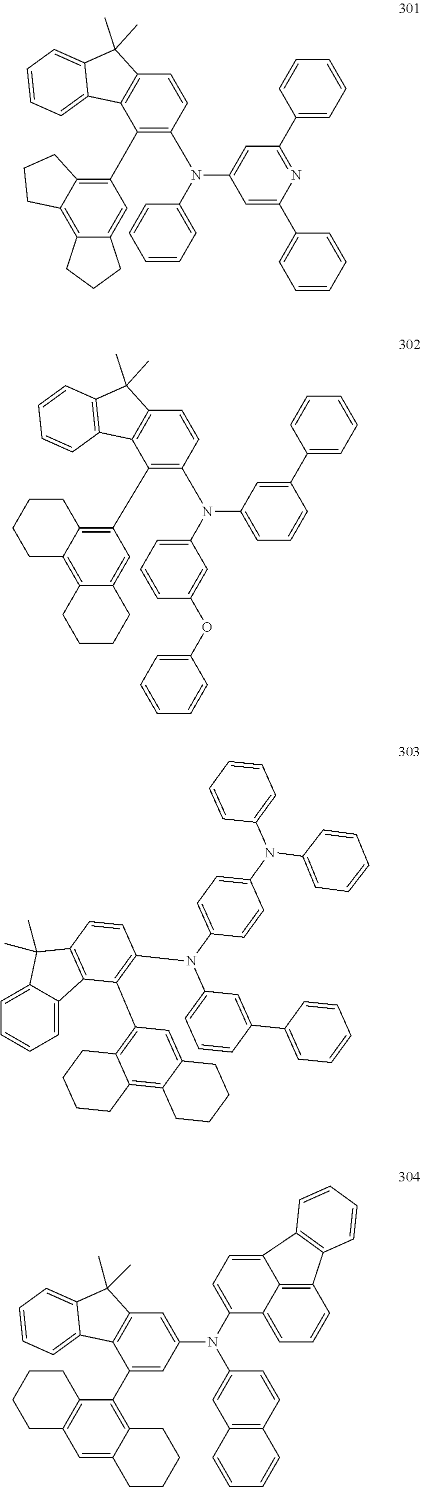 [SMILES: CC1(C)c2ccccc2-c2c1ccc(N(c1ccccc1)c1cc(-c3ccccc3)nc(-c3ccccc3)c1)c2-c1cc2c(c3c1CCC3)CCC2 ; CC1(C)c2ccccc2-c2c1ccc(N(c1cccc(Oc3ccccc3)c1)c1cccc(-c3ccccc3)c1)c2-c1cc2c(c3c1CCCC3)CCCC2 ; CC1(C)c2ccccc2-c2c1ccc(N(c1ccc(N(c3ccccc3)c3ccccc3)cc1)c1cccc(-c3ccccc3)c1)c2-c1cc2c(c3c1CCCC3)CCCC2 ; CC1(C)c2ccccc2-c2c(-c3c4c(cc5c3CCCC5)CCCC4)cc(N(c3ccc4ccccc4c3)c3ccc4c5c(cccc35)-c3ccccc3-4)cc21]

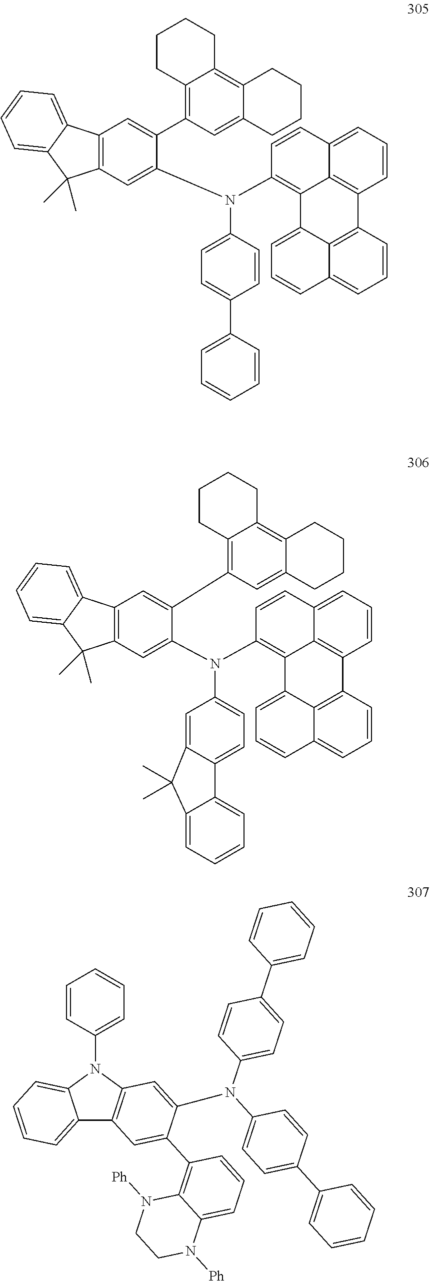 [SMILES: CC1(C)c2ccccc2-c2cc(-c3cc4c(c5c3CCCC5)CCCC4)c(N(c3ccc(-c4ccccc4)cc3)c3ccc4cccc5c6cccc7cccc(c3c45)c76)cc21 ; CC1(C)c2ccccc2-c2ccc(N(c3cc4c(cc3-c3cc5c(c6c3CCCC6)CCCC5)-c3ccccc3C4(C)C)c3ccc4cccc5c6cccc7cccc(c3c45)c76)cc21 ; c1ccc(-c2ccc(N(c3ccc(-c4ccccc4)cc3)c3cc4c(cc3-c3cccc5c3N(c3ccccc3)CCN5c3ccccc3)c3ccccc3n4-c3ccccc3)cc2)cc1]

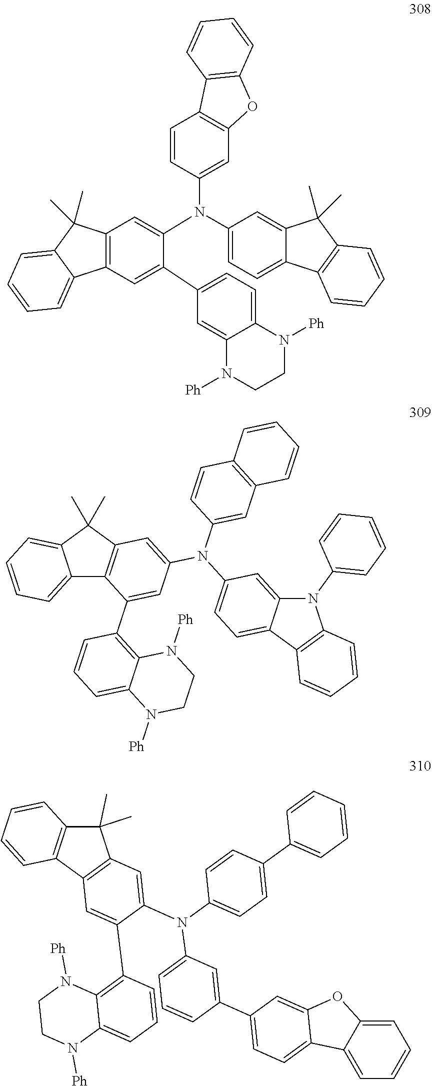

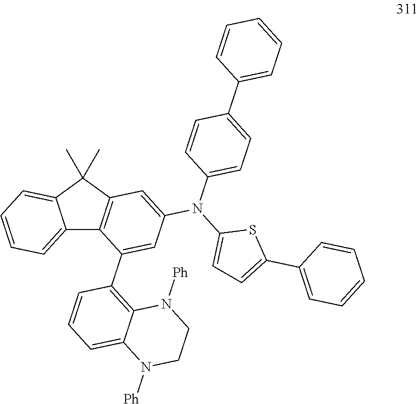 [SMILES: CC1(C)c2ccccc2-c2c(-c3cccc4c3N(c3ccccc3)CCN4c3ccccc3)cc(N(c3ccc(-c4ccccc4)cc3)c3ccc(-c4ccccc4)s3)cc21]

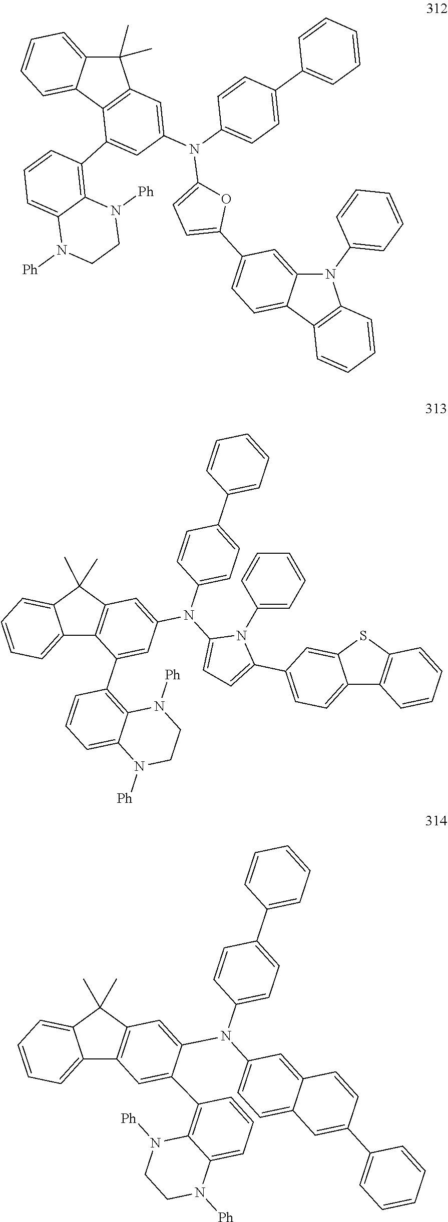 [SMILES: CC1(C)c2ccccc2-c2c(-c3cccc4c3N(c3ccccc3)CCN4c3ccccc3)cc(N(c3ccc(-c4ccccc4)cc3)c3ccc(-c4ccc5c6ccccc6n(-c6ccccc6)c5c4)o3)cc21 ; CC1(C)c2ccccc2-c2c(-c3cccc4c3N(c3ccccc3)CCN4c3ccccc3)cc(N(c3ccc(-c4ccccc4)cc3)c3ccc(-c4ccc5c(c4)sc4ccccc45)n3-c3ccccc3)cc21 ; CC1(C)c2ccccc2-c2cc(-c3cccc4c3N(c3ccccc3)CCN4c3ccccc3)c(N(c3ccc(-c4ccccc4)cc3)c3ccc4cc(-c5ccccc5)ccc4c3)cc21]

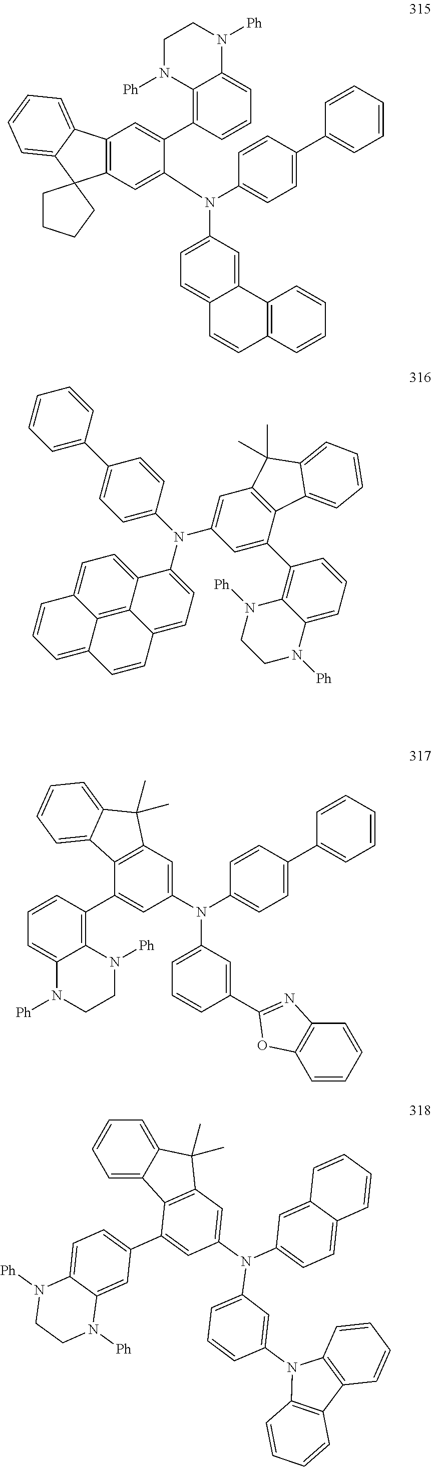 [SMILES: c1ccc(-c2ccc(N(c3ccc4ccc5ccccc5c4c3)c3cc4c(cc3-c3cccc5c3N(c3ccccc3)CCN5c3ccccc3)-c3ccccc3C43CCCC3)cc2)cc1 ; CC1(C)c2ccccc2-c2c(-c3cccc4c3N(c3ccccc3)CCN4c3ccccc3)cc(N(c3ccc(-c4ccccc4)cc3)c3ccc4ccc5cccc6ccc3c4c56)cc21 ; CC1(C)c2ccccc2-c2c(-c3cccc4c3N(c3ccccc3)CCN4c3ccccc3)cc(N(c3ccc(-c4ccccc4)cc3)c3cccc(-c4nc5ccccc5o4)c3)cc21 ; CC1(C)c2ccccc2-c2c(-c3ccc4c(c3)N(c3ccccc3)CCN4c3ccccc3)cc(N(c3cccc(-n4c5ccccc5c5ccccc54)c3)c3ccc4ccccc4c3)cc21]

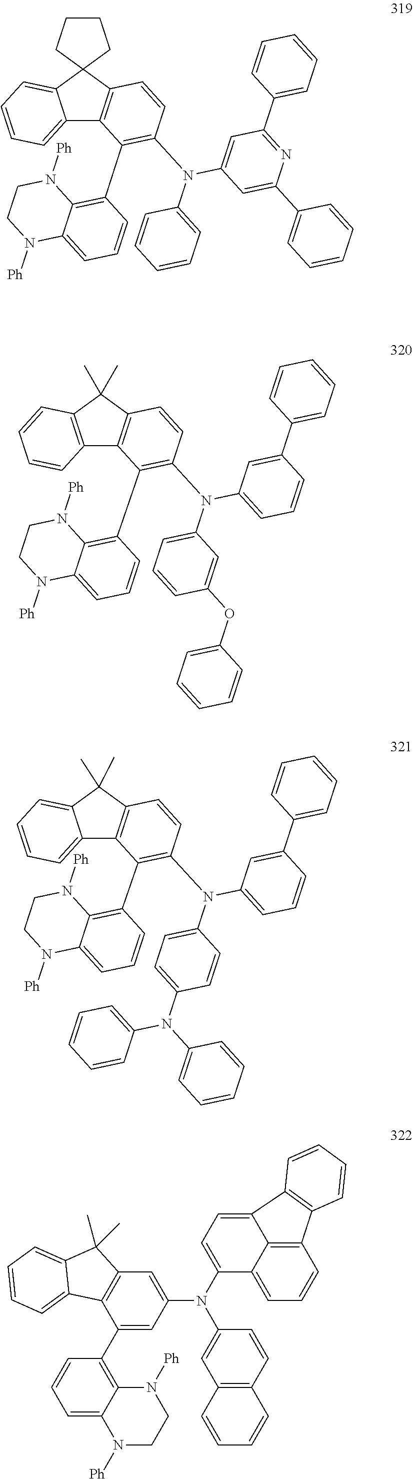 [SMILES: c1ccc(-c2cc(N(c3ccccc3)c3ccc4c(c3-c3cccc5c3N(c3ccccc3)CCN5c3ccccc3)-c3ccccc3C43CCCC3)cc(-c3ccccc3)n2)cc1 ; CC1(C)c2ccccc2-c2c1ccc(N(c1cccc(Oc3ccccc3)c1)c1cccc(-c3ccccc3)c1)c2-c1cccc2c1N(c1ccccc1)CCN2c1ccccc1 ; CC1(C)c2ccccc2-c2c1ccc(N(c1ccc(N(c3ccccc3)c3ccccc3)cc1)c1cccc(-c3ccccc3)c1)c2-c1cccc2c1N(c1ccccc1)CCN2c1ccccc1 ; CC1(C)c2ccccc2-c2c(-c3cccc4c3N(c3ccccc3)CCN4c3ccccc3)cc(N(c3ccc4ccccc4c3)c3ccc4c5c(cccc35)-c3ccccc3-4)cc21]

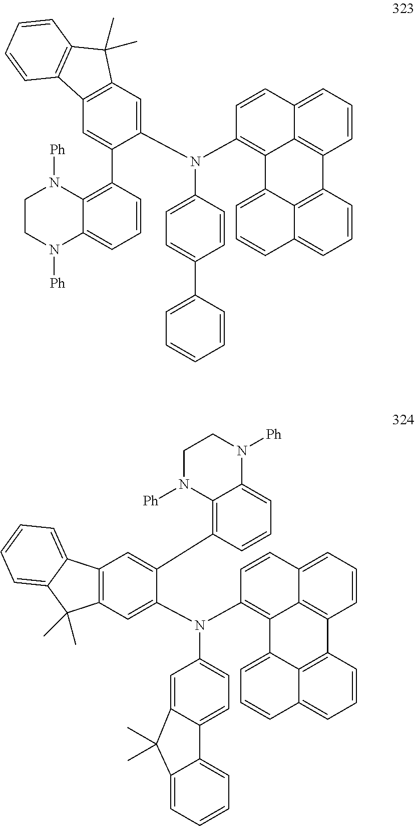 [SMILES: CC1(C)c2ccccc2-c2cc(-c3cccc4c3N(c3ccccc3)CCN4c3ccccc3)c(N(c3ccc(-c4ccccc4)cc3)c3ccc4cccc5c6cccc7cccc(c3c45)c76)cc21 ; CC1(C)c2ccccc2-c2ccc(N(c3cc4c(cc3-c3cccc5c3N(c3ccccc3)CCN5c3ccccc3)-c3ccccc3C4(C)C)c3ccc4cccc5c6cccc7cccc(c3c45)c76)cc21]

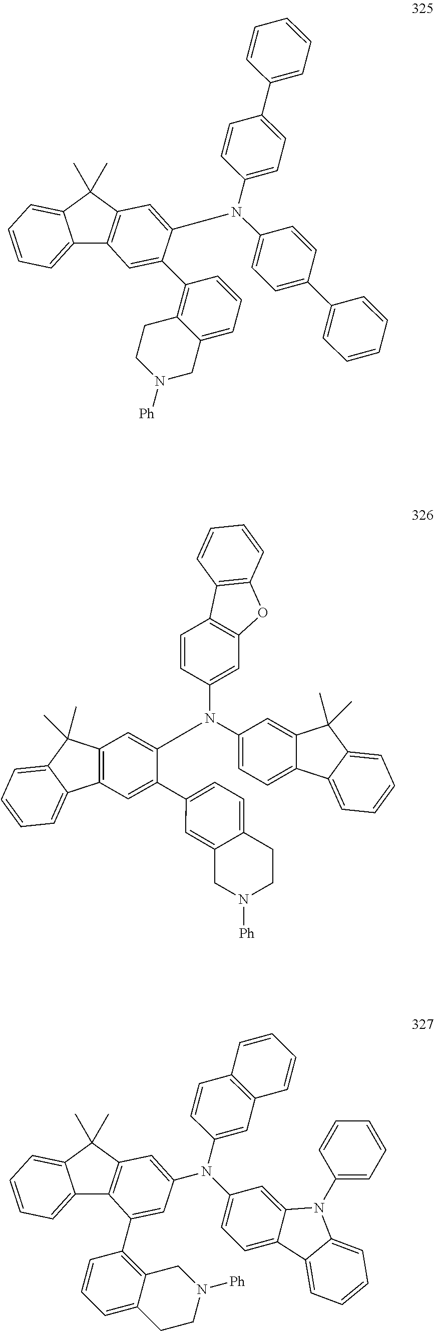 [SMILES: CC1(C)c2ccccc2-c2cc(-c3cccc4c3CCN(c3ccccc3)C4)c(N(c3ccc(-c4ccccc4)cc3)c3ccc(-c4ccccc4)cc3)cc21 ; CC1(C)c2ccccc2-c2ccc(N(c3ccc4c(c3)oc3ccccc34)c3cc4c(cc3-c3ccc5c(c3)CN(c3ccccc3)CC5)-c3ccccc3C4(C)C)cc21 ; CC1(C)c2ccccc2-c2c(-c3cccc4c3CN(c3ccccc3)CC4)cc(N(c3ccc4ccccc4c3)c3ccc4c5ccccc5n(-c5ccccc5)c4c3)cc21]

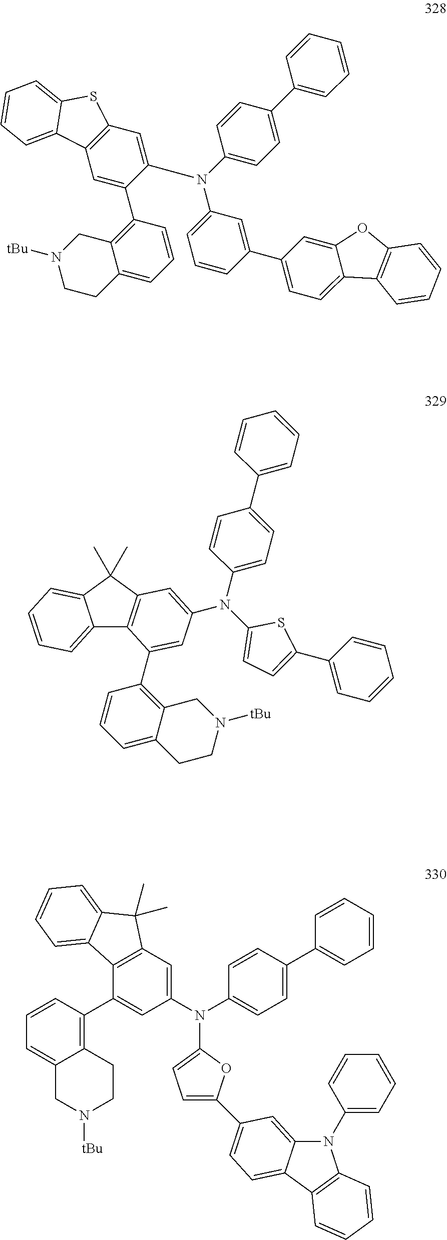 [SMILES: CC(C)(C)N1CCc2cccc(-c3cc4c(cc3N(c3ccc(-c5ccccc5)cc3)c3cccc(-c5ccc6c(c5)oc5ccccc56)c3)sc3ccccc34)c2C1 ; CC1(C)c2ccccc2-c2c(-c3cccc4c3CN(C(C)(C)C)CC4)cc(N(c3ccc(-c4ccccc4)cc3)c3ccc(-c4ccccc4)s3)cc21 ; CC1(C)c2ccccc2-c2c(-c3cccc4c3CCN(C(C)(C)C)C4)cc(N(c3ccc(-c4ccccc4)cc3)c3ccc(-c4ccc5c6ccccc6n(-c6ccccc6)c5c4)o3)cc21]

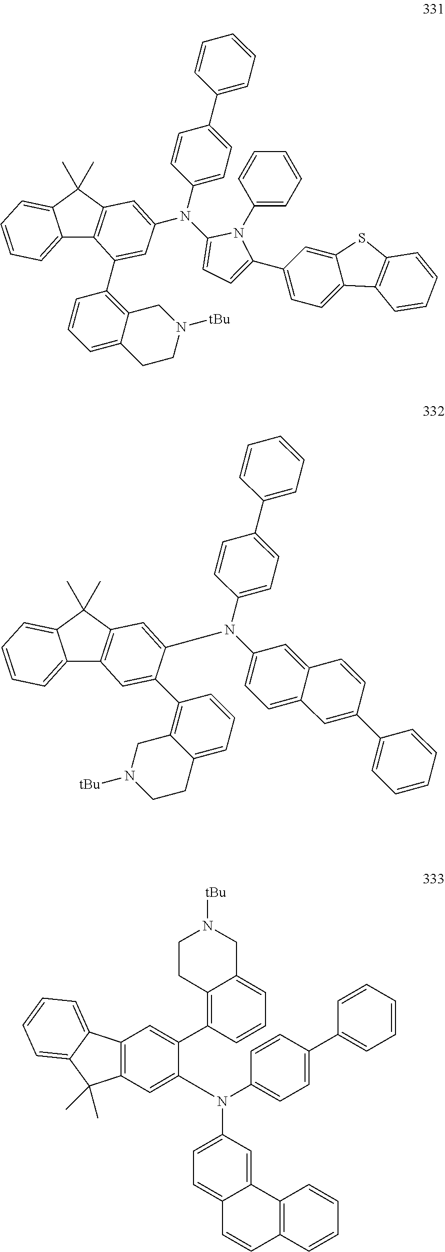 [SMILES: CC1(C)c2ccccc2-c2c(-c3cccc4c3CN(C(C)(C)C)CC4)cc(N(c3ccc(-c4ccccc4)cc3)c3ccc(-c4ccc5c(c4)sc4ccccc45)n3-c3ccccc3)cc21 ; CC1(C)c2ccccc2-c2cc(-c3cccc4c3CN(C(C)(C)C)CC4)c(N(c3ccc(-c4ccccc4)cc3)c3ccc4cc(-c5ccccc5)ccc4c3)cc21 ; CC1(C)c2ccccc2-c2cc(-c3cccc4c3CCN(C(C)(C)C)C4)c(N(c3ccc(-c4ccccc4)cc3)c3ccc4ccc5ccccc5c4c3)cc21]

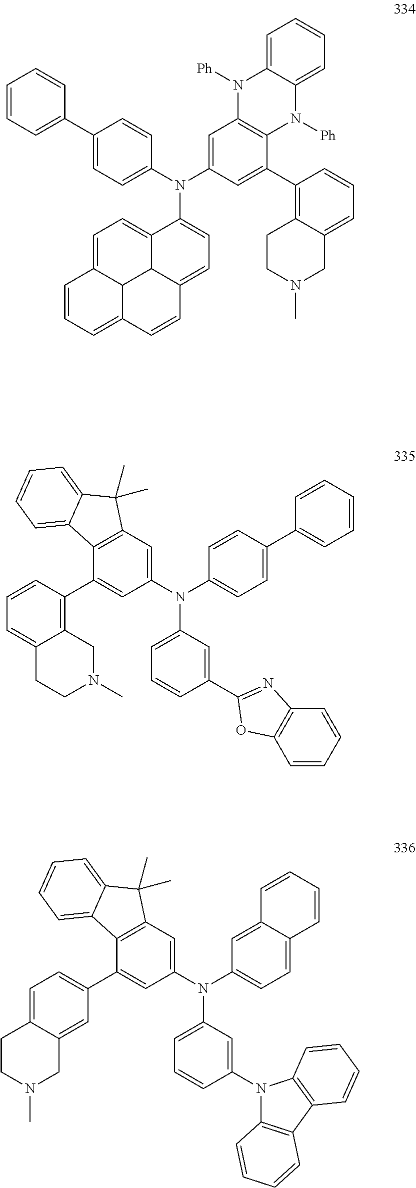 [SMILES: CN1CCc2c(cccc2-c2cc(N(C3=CC=C4C=CC5=CC=CC6=CC=C3C4C56)c3ccc(-c4ccccc4)cc3)cc3c2N(c2ccccc2)c2ccccc2N3c2ccccc2)C1 ; CN1CCc2cccc(-c3cc(N(c4ccc(-c5ccccc5)cc4)c4cccc(-c5nc6ccccc6o5)c4)cc4c3-c3ccccc3C4(C)C)c2C1 ; CN1CCc2ccc(-c3cc(N(c4cccc(-n5c6ccccc6c6ccccc65)c4)c4ccc5ccccc5c4)cc4c3-c3ccccc3C4(C)C)cc2C1]

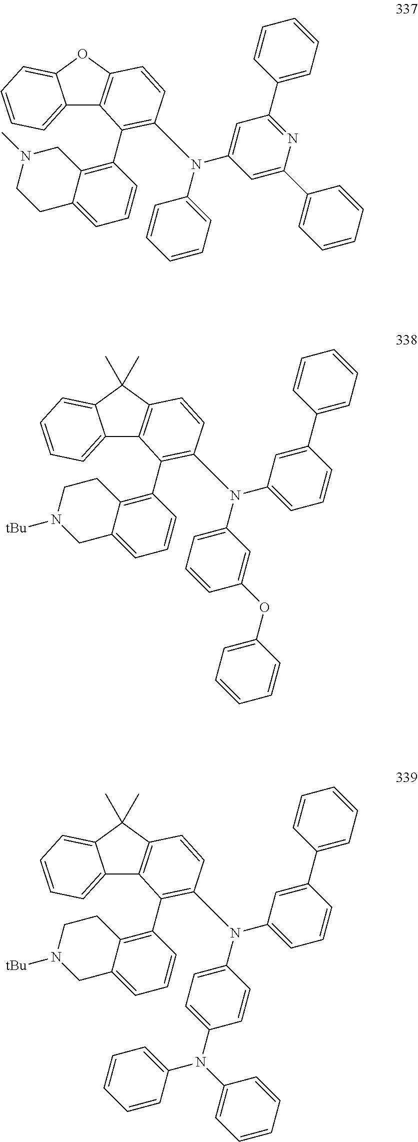 [SMILES: CN1CCc2cccc(-c3c(N(c4ccccc4)c4cc(-c5ccccc5)nc(-c5ccccc5)c4)ccc4oc5ccccc5c34)c2C1 ; CC1(C)c2ccccc2-c2c1ccc(N(c1cccc(Oc3ccccc3)c1)c1cccc(-c3ccccc3)c1)c2-c1cccc2c1CCN(C(C)(C)C)C2 ; CC1(C)c2ccccc2-c2c1ccc(N(c1ccc(N(c3ccccc3)c3ccccc3)cc1)c1cccc(-c3ccccc3)c1)c2-c1cccc2c1CCN(C(C)(C)C)C2]

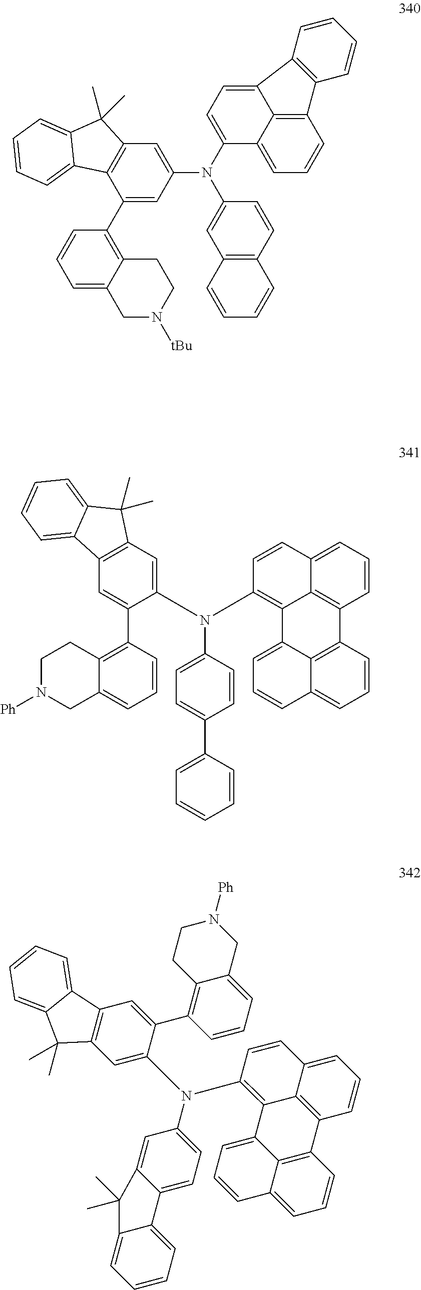 [SMILES: CC1(C)c2ccccc2-c2c(-c3cccc4c3CCN(C(C)(C)C)C4)cc(N(c3ccc4ccccc4c3)c3ccc4c5c(cccc35)-c3ccccc3-4)cc21 ; CC1(C)c2ccccc2-c2cc(-c3cccc4c3CCN(c3ccccc3)C4)c(N(c3ccc(-c4ccccc4)cc3)c3ccc4cccc5c6cccc7cccc(c3c45)c76)cc21 ; CC1(C)c2ccccc2-c2ccc(N(c3cc4c(cc3-c3cccc5c3CCN(c3ccccc3)C5)-c3ccccc3C4(C)C)c3ccc4cccc5c6cccc7cccc(c3c45)c76)cc21]

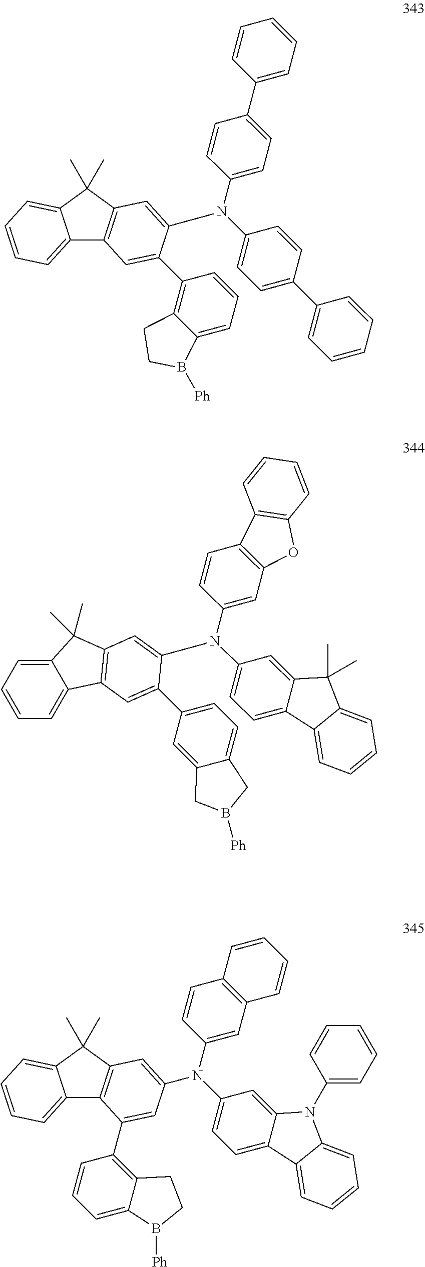 [SMILES: CC1(C)c2ccccc2-c2cc(-c3cccc4c3CCB4c3ccccc3)c(N(c3ccc(-c4ccccc4)cc3)c3ccc(-c4ccccc4)cc3)cc21 ; CC1(C)c2ccccc2-c2ccc(N(c3ccc4c(c3)oc3ccccc34)c3cc4c(cc3-c3ccc5c(c3)CB(c3ccccc3)C5)-c3ccccc3C4(C)C)cc21 ; CC1(C)c2ccccc2-c2c(-c3cccc4c3CCB4c3ccccc3)cc(N(c3ccc4ccccc4c3)c3ccc4c5ccccc5n(-c5ccccc5)c4c3)cc21]

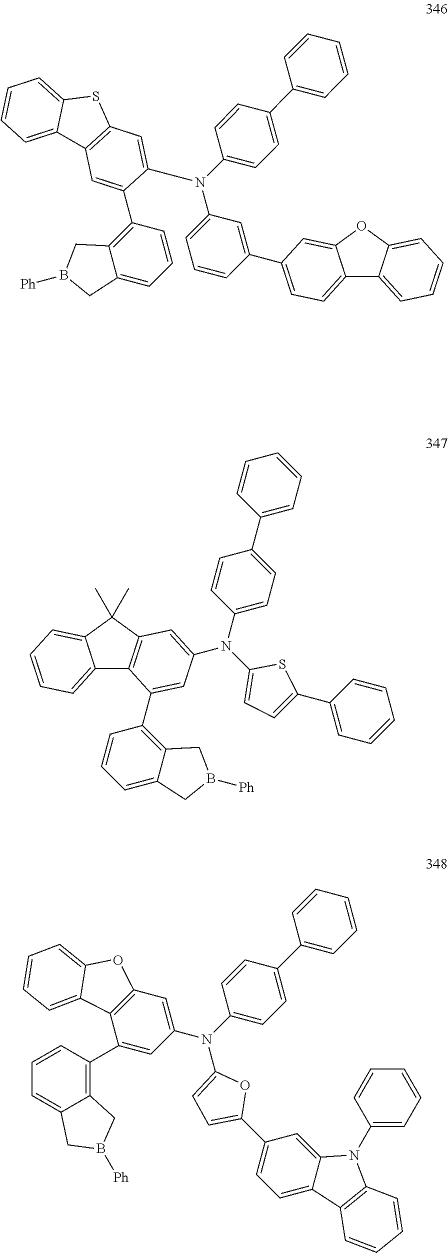 [SMILES: c1ccc(B2Cc3cccc(-c4cc5c(cc4N(c4ccc(-c6ccccc6)cc4)c4cccc(-c6ccc7c(c6)oc6ccccc67)c4)sc4ccccc45)c3C2)cc1 ; CC1(C)c2ccccc2-c2c(-c3cccc4c3CB(c3ccccc3)C4)cc(N(c3ccc(-c4ccccc4)cc3)c3ccc(-c4ccccc4)s3)cc21 ; c1ccc(B2Cc3cccc(-c4cc(N(c5ccc(-c6ccccc6)cc5)c5ccc(-c6ccc7c8ccccc8n(-c8ccccc8)c7c6)o5)cc5oc6ccccc6c45)c3C2)cc1]

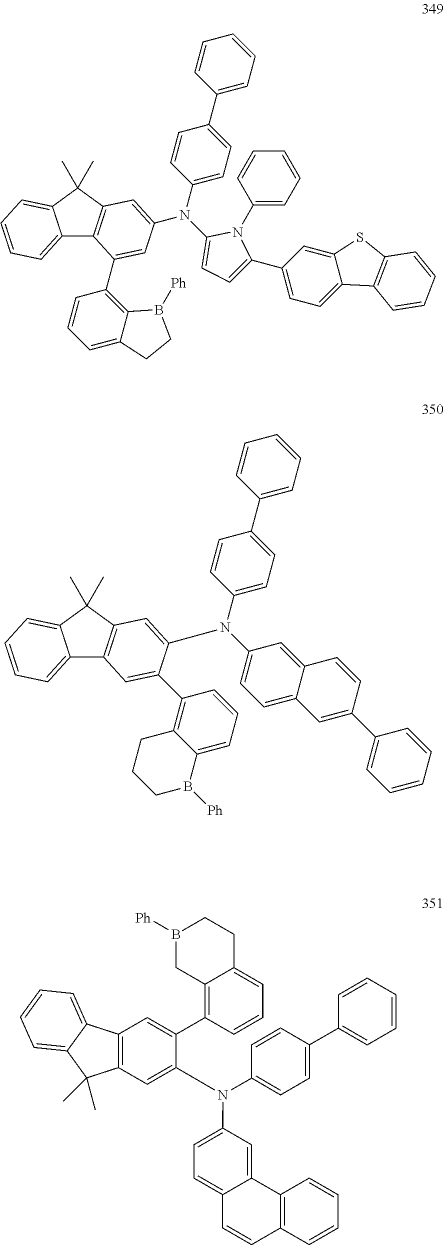 [SMILES: CC1(C)c2ccccc2-c2c(-c3cccc4c3B(c3ccccc3)CC4)cc(N(c3ccc(-c4ccccc4)cc3)c3ccc(-c4ccc5c(c4)sc4ccccc45)n3-c3ccccc3)cc21 ; CC1(C)c2ccccc2-c2cc(-c3cccc4c3CCCB4c3ccccc3)c(N(c3ccc(-c4ccccc4)cc3)c3ccc4cc(-c5ccccc5)ccc4c3)cc21 ; CC1(C)c2ccccc2-c2cc(-c3cccc4c3CB(c3ccccc3)CC4)c(N(c3ccc(-c4ccccc4)cc3)c3ccc4ccc5ccccc5c4c3)cc21]

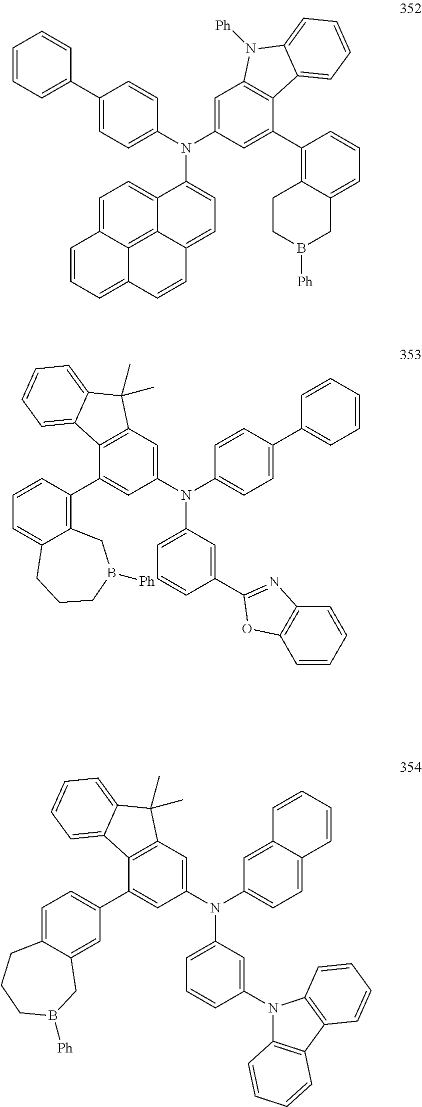 [SMILES: c1ccc(B2CCc3c(cccc3-c3cc(N(c4ccc(-c5ccccc5)cc4)c4ccc5ccc6cccc7ccc4c5c67)cc4c3c3ccccc3n4-c3ccccc3)C2)cc1 ; CC1(C)c2ccccc2-c2c(-c3cccc4c3CB(c3ccccc3)CCC4)cc(N(c3ccc(-c4ccccc4)cc3)c3cccc(-c4nc5ccccc5o4)c3)cc21 ; CC1(C)c2ccccc2-c2c(-c3ccc4c(c3)CB(c3ccccc3)CCC4)cc(N(c3cccc(-n4c5ccccc5c5ccccc54)c3)c3ccc4ccccc4c3)cc21]

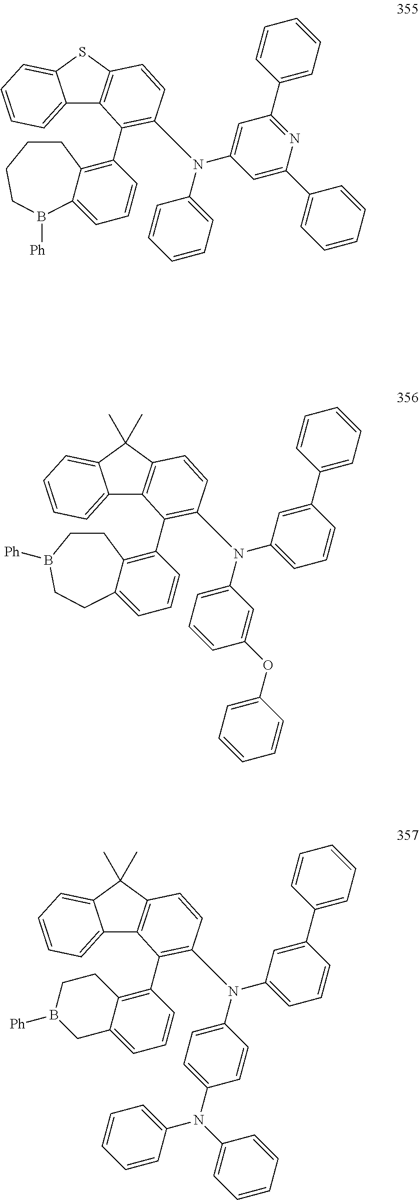 [SMILES: c1ccc(B2CCCCc3c2cccc3-c2c(N(c3ccccc3)c3cc(-c4ccccc4)nc(-c4ccccc4)c3)ccc3sc4ccccc4c23)cc1 ; CC1(C)c2ccccc2-c2c1ccc(N(c1cccc(Oc3ccccc3)c1)c1cccc(-c3ccccc3)c1)c2-c1cccc2c1CCB(c1ccccc1)CC2 ; CC1(C)c2ccccc2-c2c1ccc(N(c1ccc(N(c3ccccc3)c3ccccc3)cc1)c1cccc(-c3ccccc3)c1)c2-c1cccc2c1CCB(c1ccccc1)C2]

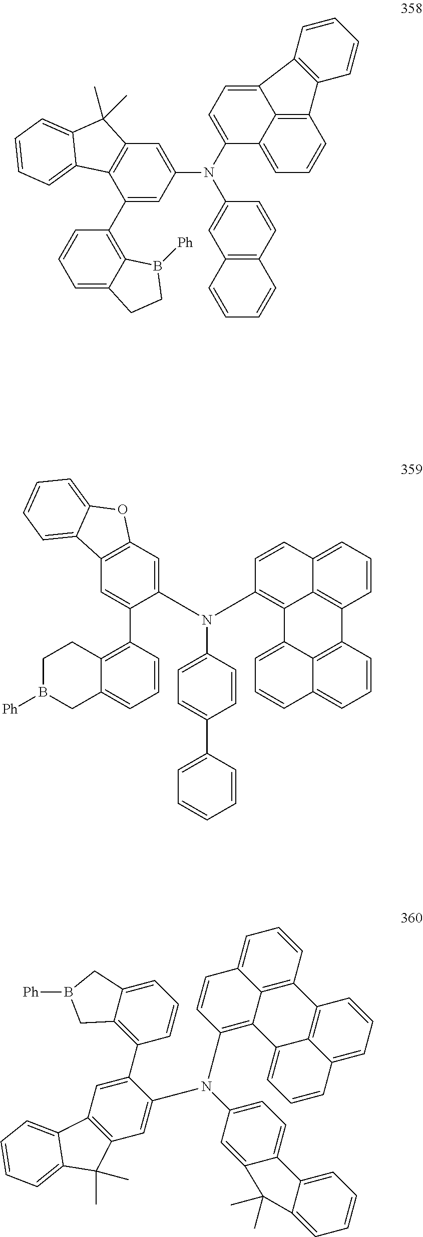 [SMILES: CC1(C)c2ccccc2-c2c(-c3cccc4c3B(c3ccccc3)CC4)cc(N(c3ccc4ccccc4c3)c3ccc4c5c(cccc35)-c3ccccc3-4)cc21 ; c1ccc(B2CCc3c(cccc3-c3cc4c(cc3N(c3ccc(-c5ccccc5)cc3)c3ccc5cccc6c7cccc8cccc(c3c56)c87)oc3ccccc34)C2)cc1 ; CC1(C)c2ccccc2-c2ccc(N(c3cc4c(cc3-c3cccc5c3CB(c3ccccc3)C5)-c3ccccc3C4(C)C)c3ccc4cccc5c6cccc7cccc(c3c45)c76)cc21]

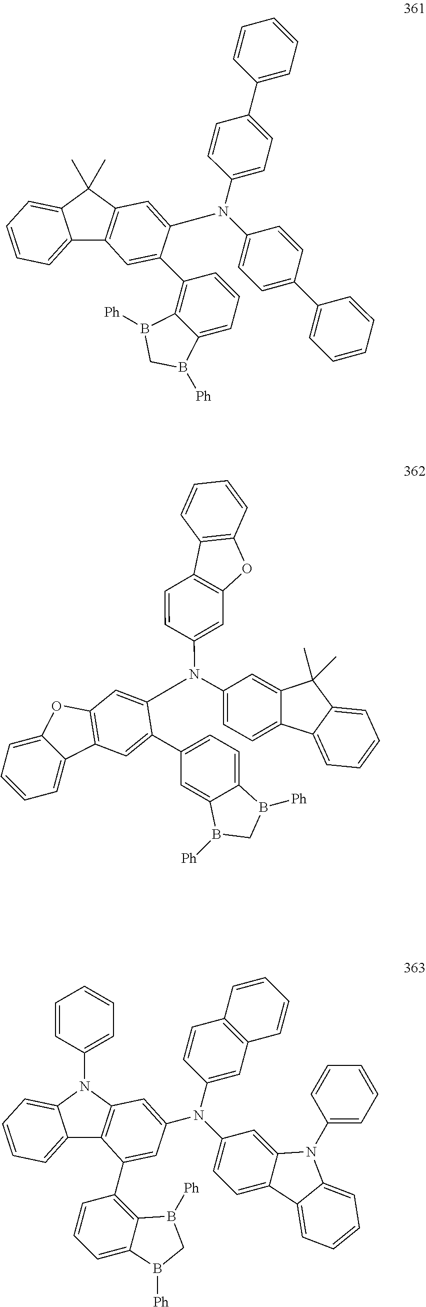 [SMILES: CC1(C)c2ccccc2-c2cc(-c3cccc4c3B(c3ccccc3)CB4c3ccccc3)c(N(c3ccc(-c4ccccc4)cc3)c3ccc(-c4ccccc4)cc3)cc21 ; CC1(C)c2ccccc2-c2ccc(N(c3ccc4c(c3)oc3ccccc34)c3cc4oc5ccccc5c4cc3-c3ccc4c(c3)B(c3ccccc3)CB4c3ccccc3)cc21 ; c1ccc(B2CB(c3ccccc3)c3c2cccc3-c2cc(N(c3ccc4ccccc4c3)c3ccc4c5ccccc5n(-c5ccccc5)c4c3)cc3c2c2ccccc2n3-c2ccccc2)cc1]

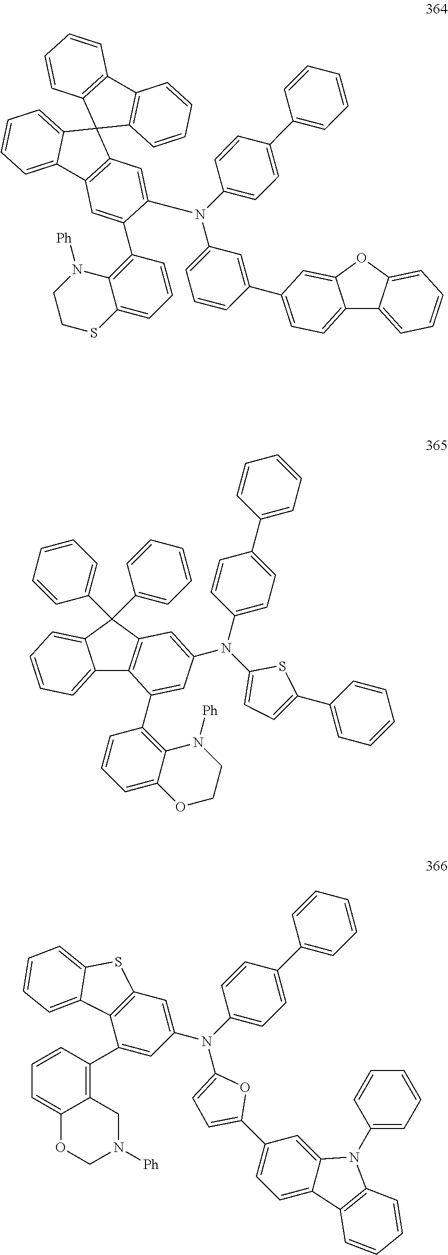 [SMILES: c1ccc(-c2ccc(N(c3cccc(-c4ccc5c(c4)oc4ccccc45)c3)c3cc4c(cc3-c3cccc5c3N(c3ccccc3)CCS5)-c3ccccc3C43c4ccccc4-c4ccccc43)cc2)cc1 ; c1ccc(-c2ccc(N(c3cc(-c4cccc5c4N(c4ccccc4)CCO5)c4c(c3)C(c3ccccc3)(c3ccccc3)c3ccccc3-4)c3ccc(-c4ccccc4)s3)cc2)cc1 ; c1ccc(-c2ccc(N(c3cc(-c4cccc5c4CN(c4ccccc4)CO5)c4c(c3)sc3ccccc34)c3ccc(-c4ccc5c6ccccc6n(-c6ccccc6)c5c4)o3)cc2)cc1]

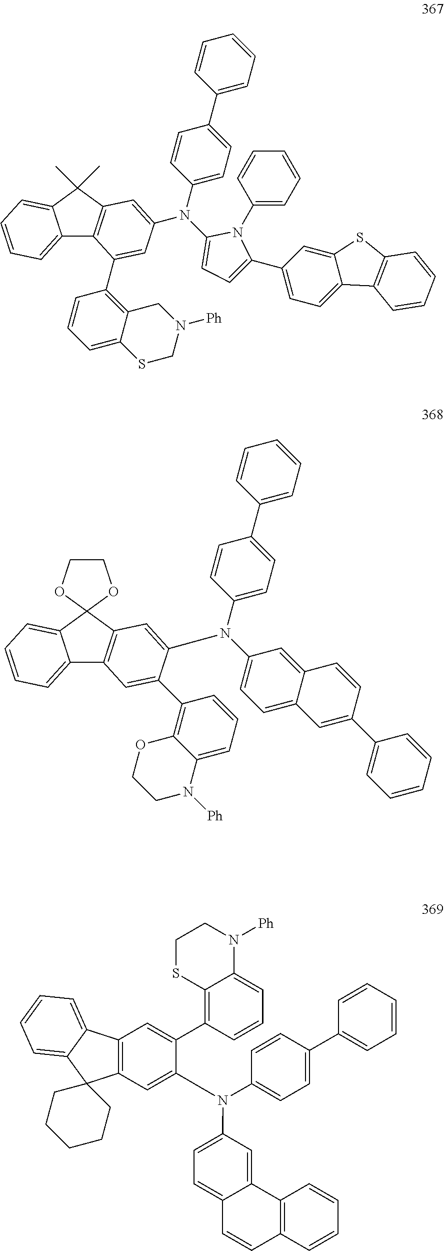 [SMILES: CC1(C)c2ccccc2-c2c(-c3cccc4c3CN(c3ccccc3)CS4)cc(N(c3ccc(-c4ccccc4)cc3)c3ccc(-c4ccc5c(c4)sc4ccccc45)n3-c3ccccc3)cc21 ; c1ccc(-c2ccc(N(c3ccc4cc(-c5ccccc5)ccc4c3)c3cc4c(cc3-c3cccc5c3OCCN5c3ccccc3)-c3ccccc3C43OCCO3)cc2)cc1 ; c1ccc(-c2ccc(N(c3ccc4ccc5ccccc5c4c3)c3cc4c(cc3-c3cccc5c3SCCN5c3ccccc3)-c3ccccc3C43CCCCC3)cc2)cc1]

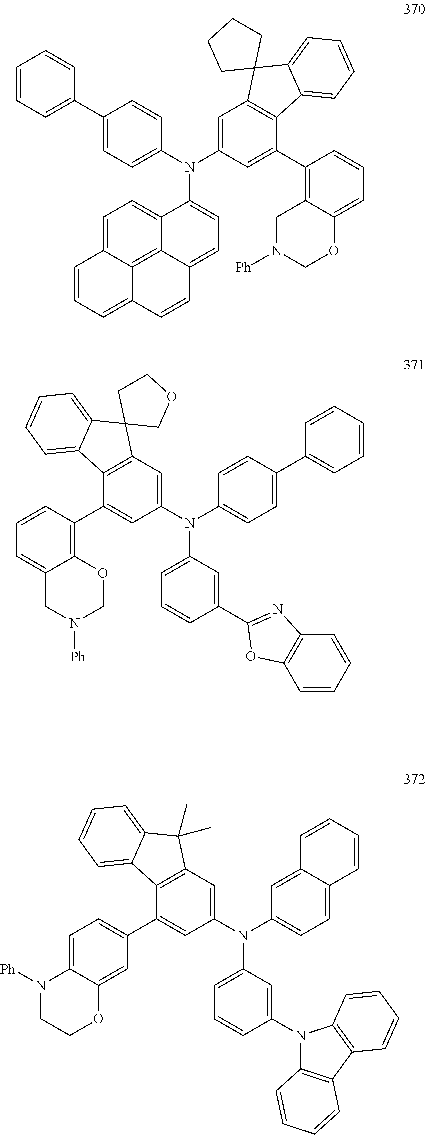 [SMILES: c1ccc(-c2ccc(N(c3cc(-c4cccc5c4CN(c4ccccc4)CO5)c4c(c3)C3(CCCC3)c3ccccc3-4)c3ccc4ccc5cccc6ccc3c4c56)cc2)cc1 ; c1ccc(-c2ccc(N(c3cccc(-c4nc5ccccc5o4)c3)c3cc(-c4cccc5c4OCN(c4ccccc4)C5)c4c(c3)C3(CCOC3)c3ccccc3-4)cc2)cc1 ; CC1(C)c2ccccc2-c2c(-c3ccc4c(c3)OCCN4c3ccccc3)cc(N(c3cccc(-n4c5ccccc5c5ccccc54)c3)c3ccc4ccccc4c3)cc21]

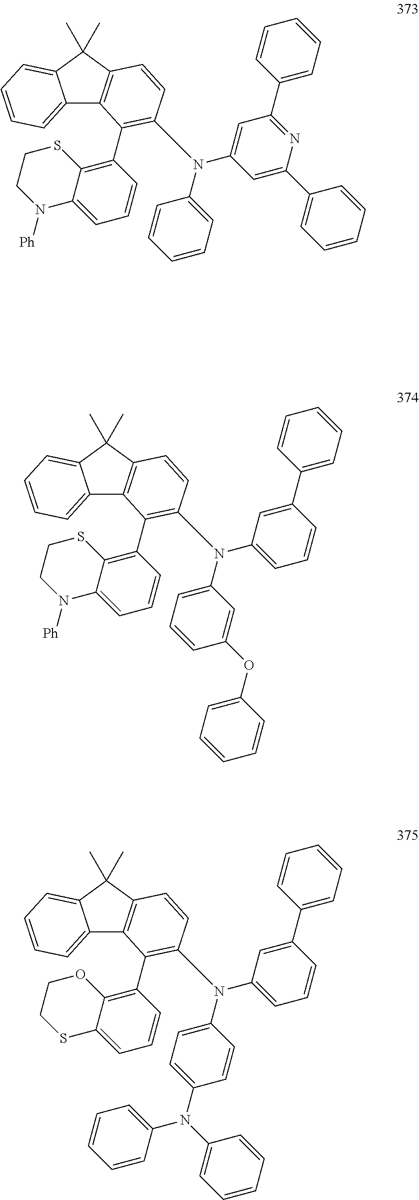 [SMILES: CC1(C)c2ccccc2-c2c1ccc(N(c1ccccc1)c1cc(-c3ccccc3)nc(-c3ccccc3)c1)c2-c1cccc2c1SCCN2c1ccccc1 ; CC1(C)c2ccccc2-c2c1ccc(N(c1cccc(Oc3ccccc3)c1)c1cccc(-c3ccccc3)c1)c2-c1cccc2c1SCCN2c1ccccc1 ; CC1(C)c2ccccc2-c2c1ccc(N(c1ccc(N(c3ccccc3)c3ccccc3)cc1)c1cccc(-c3ccccc3)c1)c2-c1cccc2c1OCCS2]

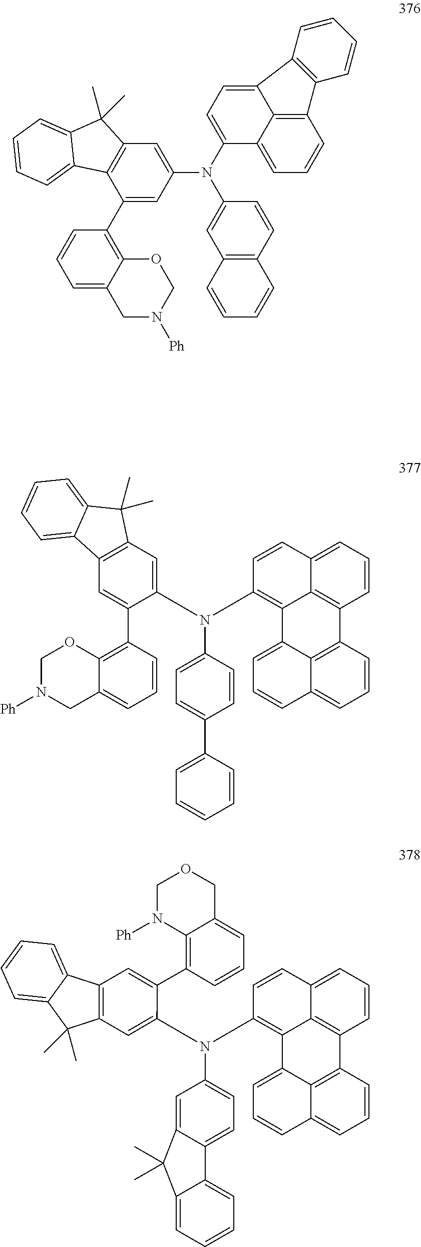 [SMILES: CC1(C)c2ccccc2-c2c(-c3cccc4c3OCN(c3ccccc3)C4)cc(N(c3ccc4ccccc4c3)c3ccc4c5c(cccc35)-c3ccccc3-4)cc21 ; CC1(C)c2ccccc2-c2cc(-c3cccc4c3OCN(c3ccccc3)C4)c(N(c3ccc(-c4ccccc4)cc3)c3ccc4cccc5c6cccc7cccc(c3c45)c76)cc21 ; CC1(C)c2ccccc2-c2ccc(N(c3cc4c(cc3-c3cccc5c3N(c3ccccc3)COC5)-c3ccccc3C4(C)C)c3ccc4cccc5c6cccc7cccc(c3c45)c76)cc21]

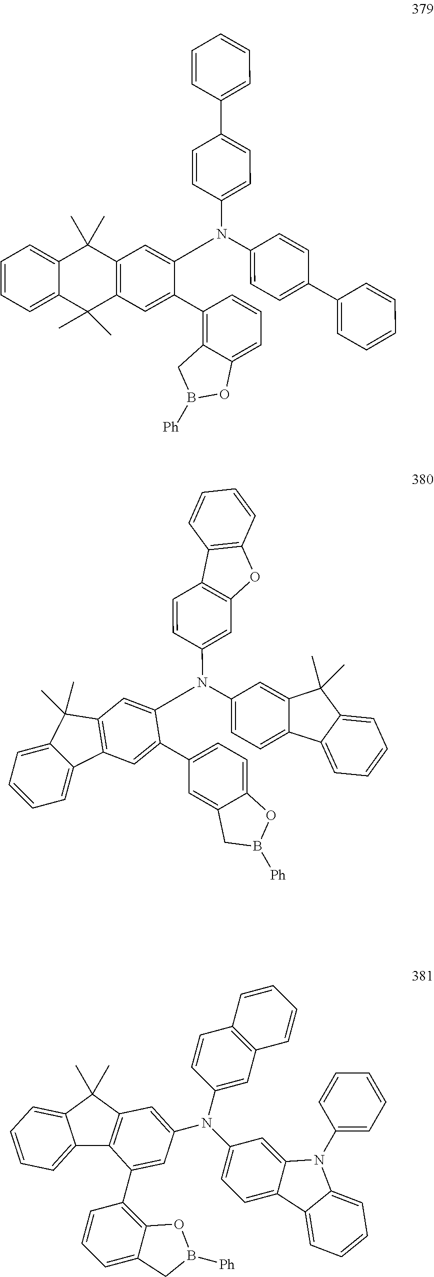 [SMILES: CC1(C)c2ccccc2C(C)(C)c2cc(N(c3ccc(-c4ccccc4)cc3)c3ccc(-c4ccccc4)cc3)c(-c3cccc4c3CB(c3ccccc3)O4)cc21 ; CC1(C)c2ccccc2-c2ccc(N(c3ccc4c(c3)oc3ccccc34)c3cc4c(cc3-c3ccc5c(c3)CB(c3ccccc3)O5)-c3ccccc3C4(C)C)cc21 ; CC1(C)c2ccccc2-c2c(-c3cccc4c3OB(c3ccccc3)C4)cc(N(c3ccc4ccccc4c3)c3ccc4c5ccccc5n(-c5ccccc5)c4c3)cc21]

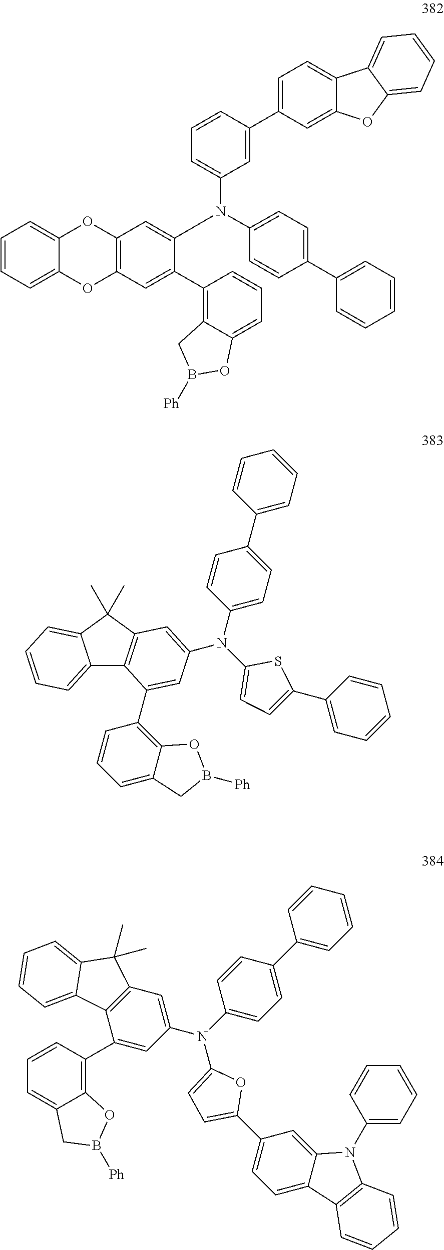 [SMILES: c1ccc(B2Cc3c(cccc3-c3cc4c(cc3N(c3ccc(-c5ccccc5)cc3)c3cccc(-c5ccc6c(c5)oc5ccccc56)c3)Oc3ccccc3O4)O2)cc1 ; CC1(C)c2ccccc2-c2c(-c3cccc4c3OB(c3ccccc3)C4)cc(N(c3ccc(-c4ccccc4)cc3)c3ccc(-c4ccccc4)s3)cc21 ; CC1(C)c2ccccc2-c2c(-c3cccc4c3OB(c3ccccc3)C4)cc(N(c3ccc(-c4ccccc4)cc3)c3ccc(-c4ccc5c6ccccc6n(-c6ccccc6)c5c4)o3)cc21]

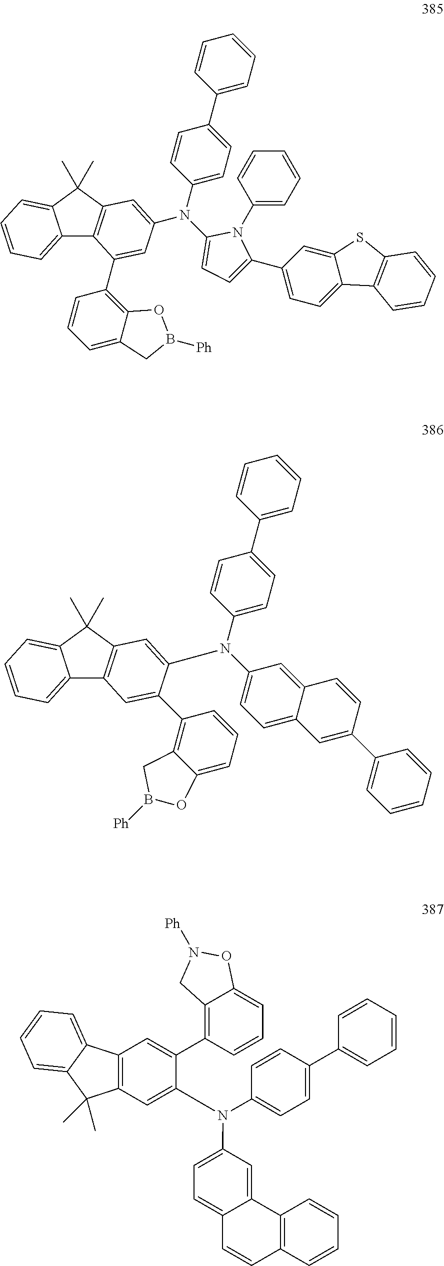 [SMILES: CC1(C)c2ccccc2-c2c(-c3cccc4c3OB(c3ccccc3)C4)cc(N(c3ccc(-c4ccccc4)cc3)c3ccc(-c4ccc5c(c4)sc4ccccc45)n3-c3ccccc3)cc21 ; CC1(C)c2ccccc2-c2cc(-c3cccc4c3CB(c3ccccc3)O4)c(N(c3ccc(-c4ccccc4)cc3)c3ccc4cc(-c5ccccc5)ccc4c3)cc21 ; CC1(C)c2ccccc2-c2cc(-c3cccc4c3CN(c3ccccc3)O4)c(N(c3ccc(-c4ccccc4)cc3)c3ccc4ccc5ccccc5c4c3)cc21]

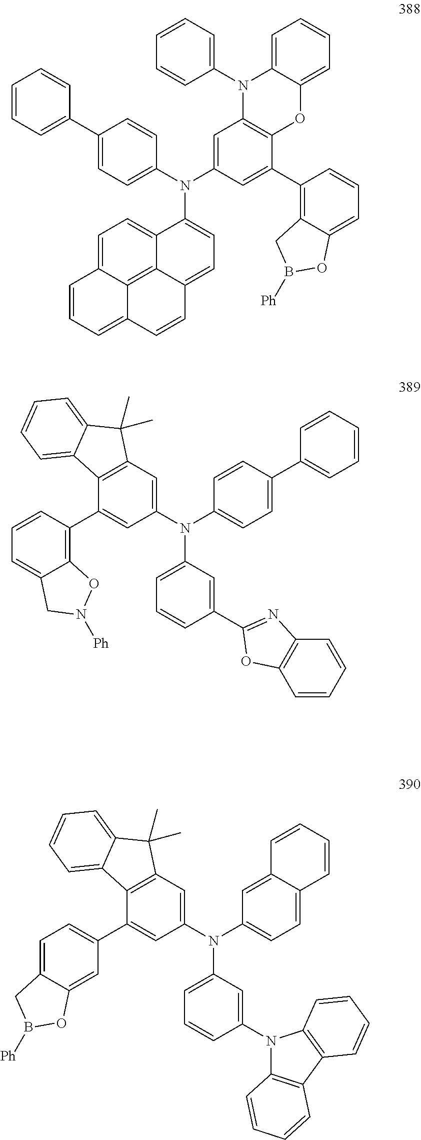 [SMILES: c1ccc(B2Cc3c(cccc3-c3cc(N(c4ccc(-c5ccccc5)cc4)c4ccc5ccc6cccc7ccc4c5c67)cc4c3Oc3ccccc3N4c3ccccc3)O2)cc1 ; CC1(C)c2ccccc2-c2c(-c3cccc4c3ON(c3ccccc3)C4)cc(N(c3ccc(-c4ccccc4)cc3)c3cccc(-c4nc5ccccc5o4)c3)cc21 ; CC1(C)c2ccccc2-c2c(-c3ccc4c(c3)OB(c3ccccc3)C4)cc(N(c3cccc(-n4c5ccccc5c5ccccc54)c3)c3ccc4ccccc4c3)cc21]

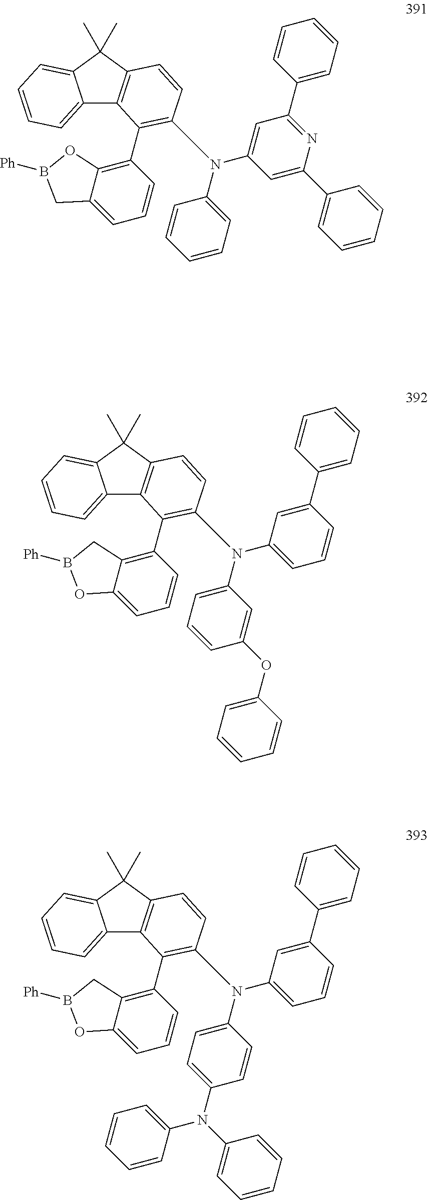 [SMILES: CC1(C)c2ccccc2-c2c1ccc(N(c1ccccc1)c1cc(-c3ccccc3)nc(-c3ccccc3)c1)c2-c1cccc2c1OB(c1ccccc1)C2 ; CC1(C)c2ccccc2-c2c1ccc(N(c1cccc(Oc3ccccc3)c1)c1cccc(-c3ccccc3)c1)c2-c1cccc2c1CB(c1ccccc1)O2 ; CC1(C)c2ccccc2-c2c1ccc(N(c1ccc(N(c3ccccc3)c3ccccc3)cc1)c1cccc(-c3ccccc3)c1)c2-c1cccc2c1CB(c1ccccc1)O2]

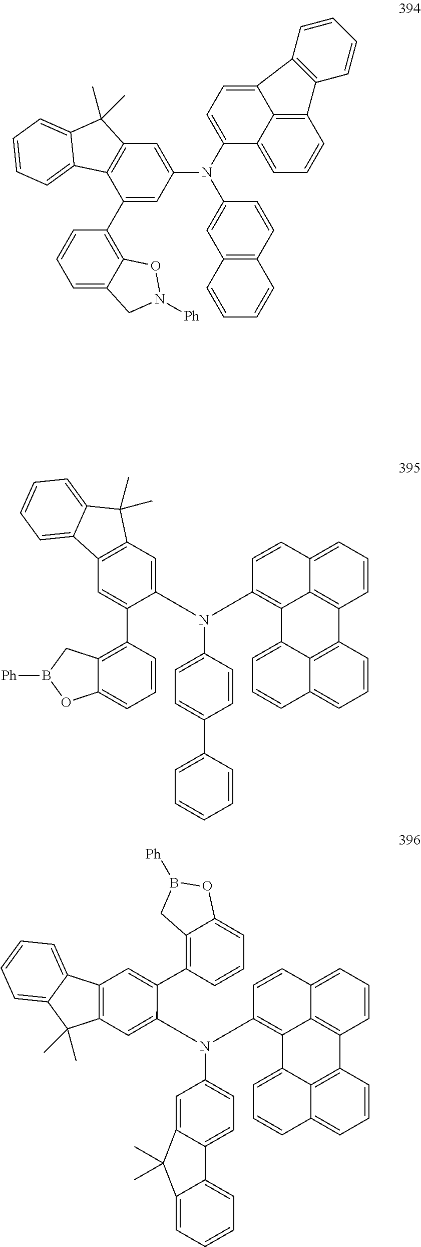 [SMILES: CC1(C)c2ccccc2-c2c(-c3cccc4c3ON(c3ccccc3)C4)cc(N(c3ccc4ccccc4c3)c3ccc4c5c(cccc35)-c3ccccc3-4)cc21 ; CC1(C)c2ccccc2-c2cc(-c3cccc4c3CB(c3ccccc3)O4)c(N(c3ccc(-c4ccccc4)cc3)c3ccc4cccc5c6cccc7cccc(c3c45)c76)cc21 ; CC1(C)c2ccccc2-c2ccc(N(c3cc4c(cc3-c3cccc5c3CB(c3ccccc3)O5)-c3ccccc3C4(C)C)c3ccc4cccc5c6cccc7cccc(c3c45)c76)cc21]

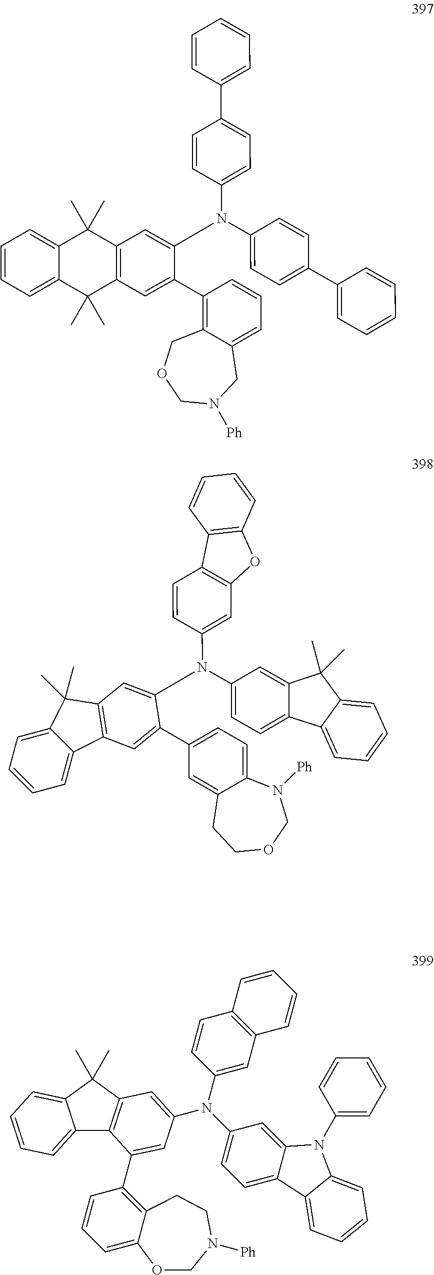

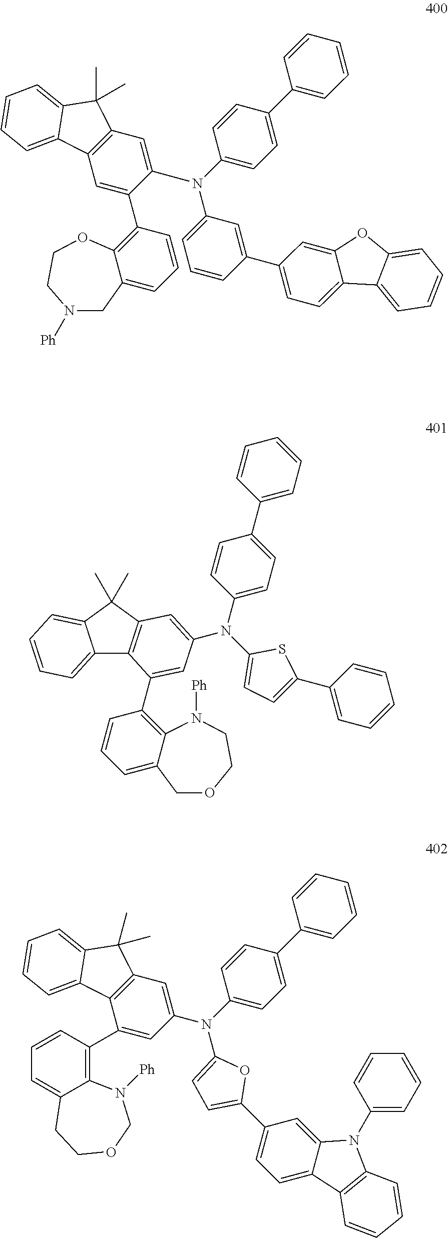 [SMILES: CC1(C)c2ccccc2-c2cc(-c3cccc4c3OCCN(c3ccccc3)C4)c(N(c3ccc(-c4ccccc4)cc3)c3cccc(-c4ccc5c(c4)oc4ccccc45)c3)cc21 ; CC1(C)c2ccccc2-c2c(-c3cccc4c3N(c3ccccc3)CCOC4)cc(N(c3ccc(-c4ccccc4)cc3)c3ccc(-c4ccccc4)s3)cc21 ; CC1(C)c2ccccc2-c2c(-c3cccc4c3N(c3ccccc3)COCC4)cc(N(c3ccc(-c4ccccc4)cc3)c3ccc(-c4ccc5c6ccccc6n(-c6ccccc6)c5c4)o3)cc21]

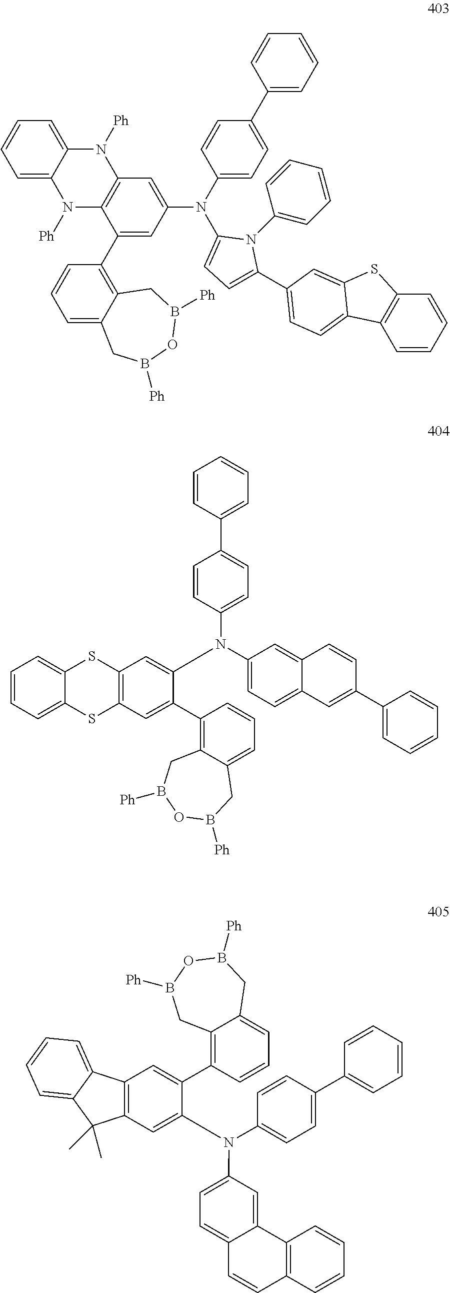 [SMILES: c1ccc(B2Cc3cccc(-c4cc(N(c5ccc(-c6ccccc6)cc5)c5ccc(-c6ccc7c(c6)sc6ccccc67)n5-c5ccccc5)cc5c4N(c4ccccc4)c4ccccc4N5c4ccccc4)c3CB(c3ccccc3)O2)cc1 ; c1ccc(B2Cc3cccc(-c4cc5c(cc4N(c4ccc(-c6ccccc6)cc4)c4ccc6cc(-c7ccccc7)ccc6c4)Sc4ccccc4S5)c3CB(c3ccccc3)O2)cc1 ; CC1(C)c2ccccc2-c2cc(-c3cccc4c3CB(c3ccccc3)OB(c3ccccc3)C4)c(N(c3ccc(-c4ccccc4)cc3)c3ccc4ccc5ccccc5c4c3)cc21]

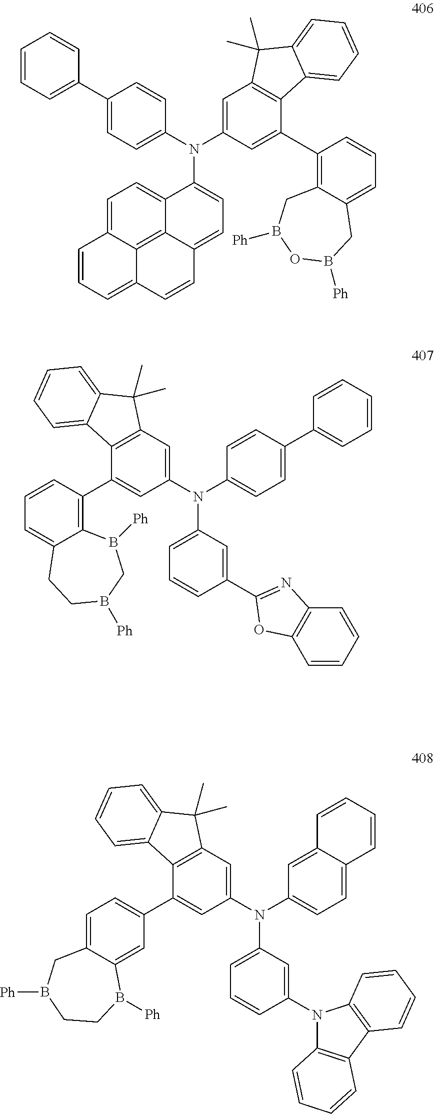 [SMILES: CC1(C)c2ccccc2-c2c(-c3cccc4c3CB(c3ccccc3)OB(c3ccccc3)C4)cc(N(c3ccc(-c4ccccc4)cc3)c3ccc4ccc5cccc6ccc3c4c56)cc21 ; CC1(C)c2ccccc2-c2c(-c3cccc4c3B(c3ccccc3)CB(c3ccccc3)CC4)cc(N(c3ccc(-c4ccccc4)cc3)c3cccc(-c4nc5ccccc5o4)c3)cc21 ; CC1(C)c2ccccc2-c2c(-c3ccc4c(c3)B(c3ccccc3)CCB(c3ccccc3)C4)cc(N(c3cccc(-n4c5ccccc5c5ccccc54)c3)c3ccc4ccccc4c3)cc21]

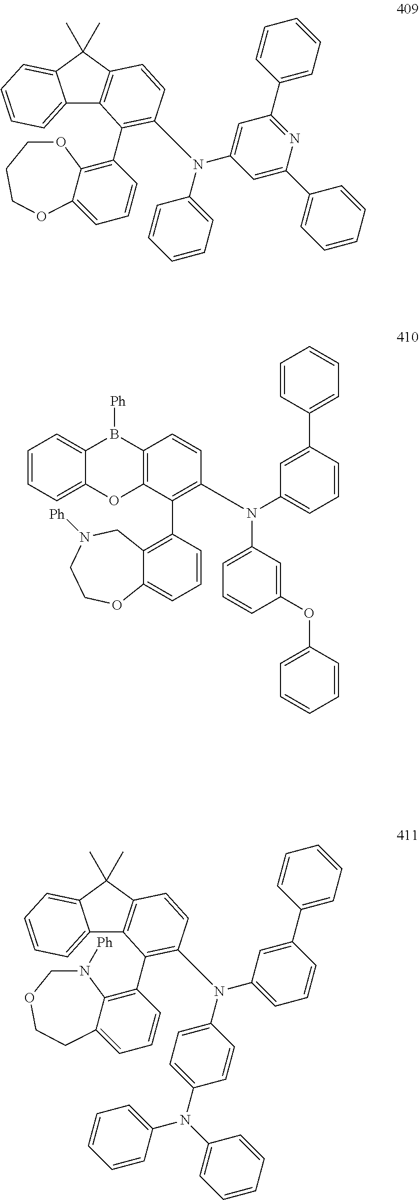 [SMILES: CC1(C)c2ccccc2-c2c1ccc(N(c1ccccc1)c1cc(-c3ccccc3)nc(-c3ccccc3)c1)c2-c1cccc2c1OCCCO2 ; c1ccc(Oc2cccc(N(c3cccc(-c4ccccc4)c3)c3ccc4c(c3-c3cccc5c3CN(c3ccccc3)CCO5)Oc3ccccc3B4c3ccccc3)c2)cc1 ; CC1(C)c2ccccc2-c2c1ccc(N(c1ccc(N(c3ccccc3)c3ccccc3)cc1)c1cccc(-c3ccccc3)c1)c2-c1cccc2c1N(c1ccccc1)COCC2]

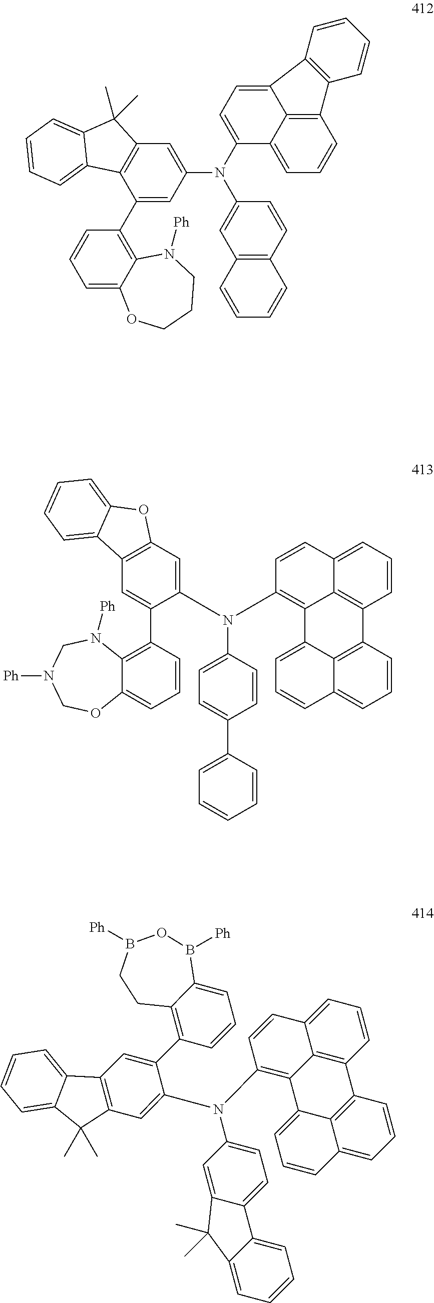 [SMILES: CC1(C)c2ccccc2-c2c(-c3cccc4c3N(c3ccccc3)CCCO4)cc(N(c3ccc4ccccc4c3)c3ccc4c5c(cccc35)-c3ccccc3-4)cc21 ; c1ccc(-c2ccc(N(c3cc4oc5ccccc5c4cc3-c3cccc4c3N(c3ccccc3)CN(c3ccccc3)CO4)c3ccc4cccc5c6cccc7cccc(c3c45)c76)cc2)cc1 ; CC1(C)c2ccccc2-c2ccc(N(c3cc4c(cc3-c3cccc5c3CCB(c3ccccc3)OB5c3ccccc3)-c3ccccc3C4(C)C)c3ccc4cccc5c6cccc7cccc(c3c45)c76)cc21]

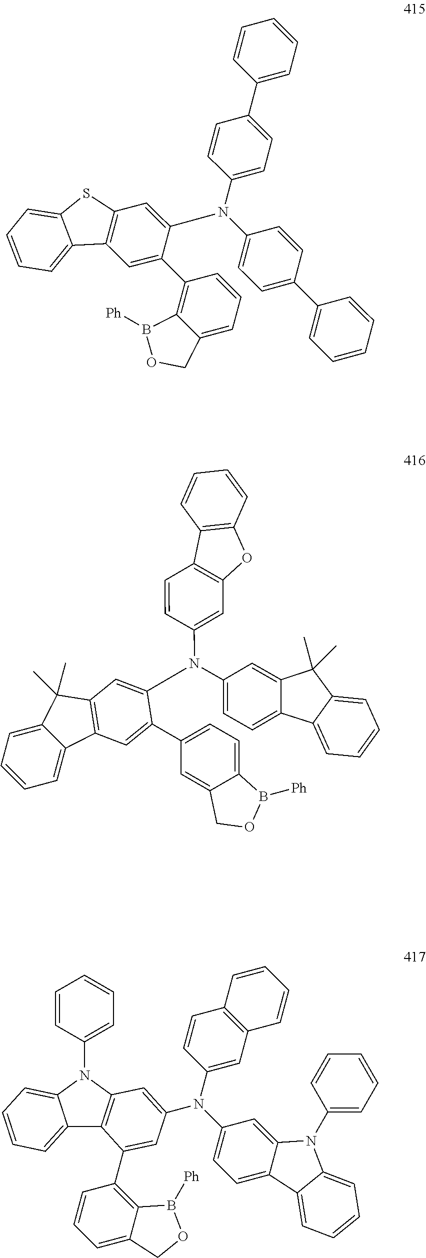 [SMILES: c1ccc(B2OCc3cccc(-c4cc5c(cc4N(c4ccc(-c6ccccc6)cc4)c4ccc(-c6ccccc6)cc4)sc4ccccc45)c32)cc1 ; CC1(C)c2ccccc2-c2ccc(N(c3ccc4c(c3)oc3ccccc34)c3cc4c(cc3-c3ccc5c(c3)COB5c3ccccc3)-c3ccccc3C4(C)C)cc21 ; c1ccc(B2OCc3cccc(-c4cc(N(c5ccc6ccccc6c5)c5ccc6c7ccccc7n(-c7ccccc7)c6c5)cc5c4c4ccccc4n5-c4ccccc4)c32)cc1]

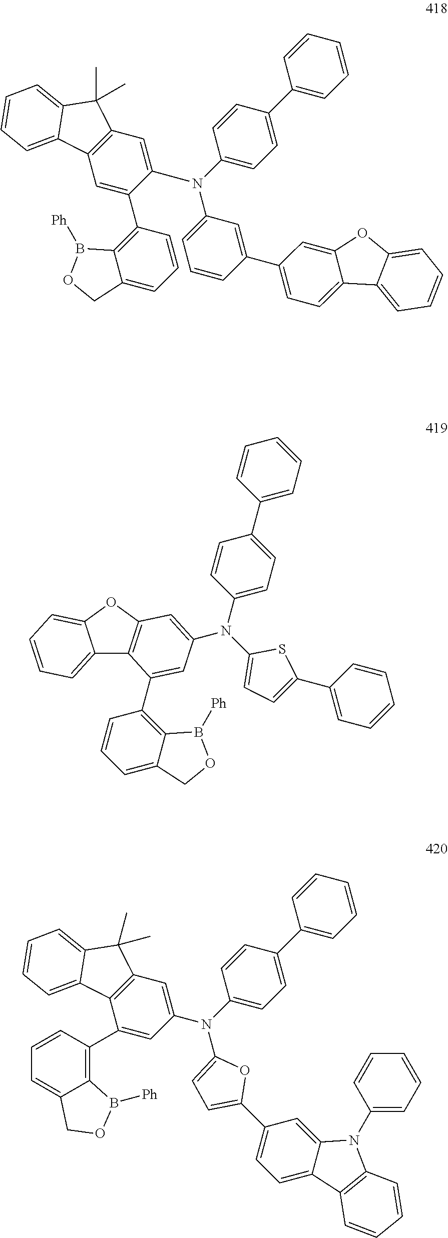 [SMILES: CC1(C)c2ccccc2-c2cc(-c3cccc4c3B(c3ccccc3)OC4)c(N(c3ccc(-c4ccccc4)cc3)c3cccc(-c4ccc5c(c4)oc4ccccc45)c3)cc21 ; c1ccc(B2OCc3cccc(-c4cc(N(c5ccc(-c6ccccc6)cc5)c5ccc(-c6ccccc6)s5)cc5oc6ccccc6c45)c32)cc1 ; CC1(C)c2ccccc2-c2c(-c3cccc4c3B(c3ccccc3)OC4)cc(N(c3ccc(-c4ccccc4)cc3)c3ccc(-c4ccc5c6ccccc6n(-c6ccccc6)c5c4)o3)cc21]

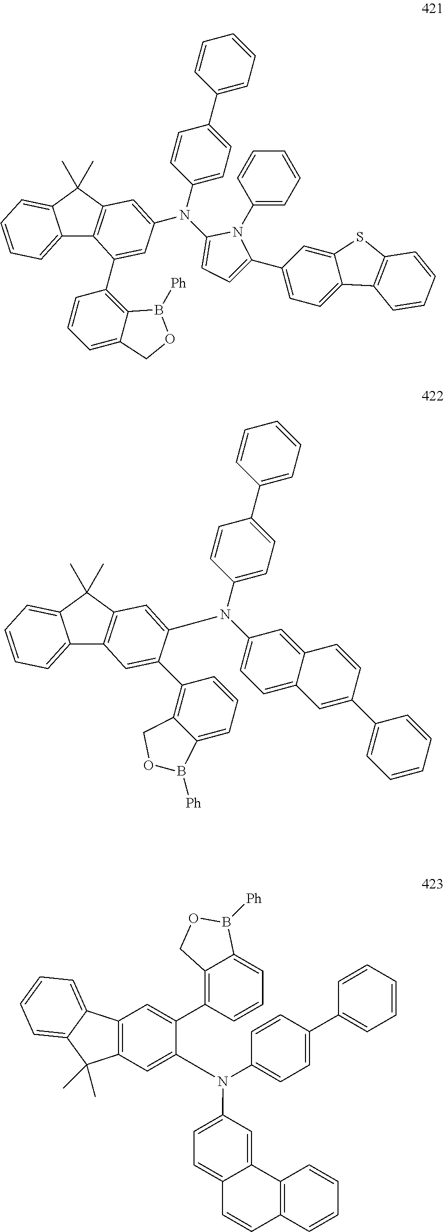 [SMILES: CC1(C)c2ccccc2-c2c(-c3cccc4c3B(c3ccccc3)OC4)cc(N(c3ccc(-c4ccccc4)cc3)c3ccc(-c4ccc5c(c4)sc4ccccc45)n3-c3ccccc3)cc21 ; CC1(C)c2ccccc2-c2cc(-c3cccc4c3COB4c3ccccc3)c(N(c3ccc(-c4ccccc4)cc3)c3ccc4cc(-c5ccccc5)ccc4c3)cc21 ; CC1(C)c2ccccc2-c2cc(-c3cccc4c3COB4c3ccccc3)c(N(c3ccc(-c4ccccc4)cc3)c3ccc4ccc5ccccc5c4c3)cc21]

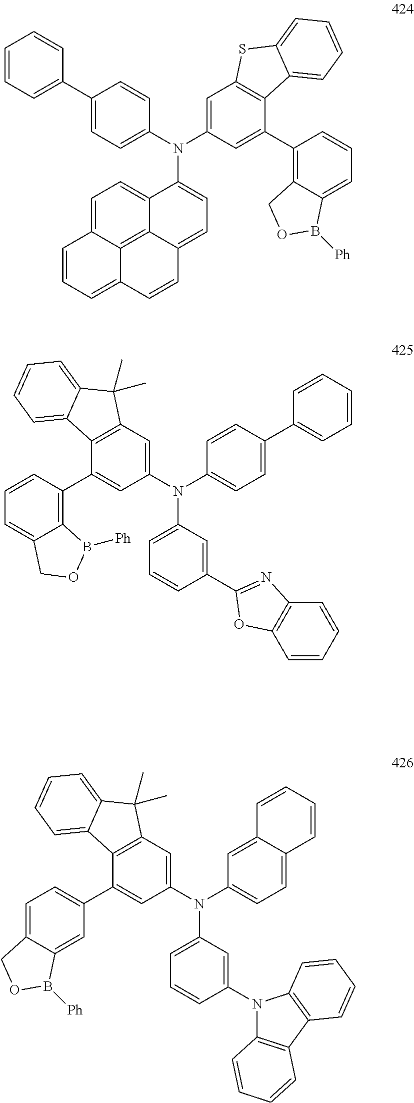 [SMILES: c1ccc(B2OCc3c2cccc3-c2cc(N(c3ccc(-c4ccccc4)cc3)c3ccc4ccc5cccc6ccc3c4c56)cc3sc4ccccc4c23)cc1 ; CC1(C)c2ccccc2-c2c(-c3cccc4c3B(c3ccccc3)OC4)cc(N(c3ccc(-c4ccccc4)cc3)c3cccc(-c4nc5ccccc5o4)c3)cc21 ; CC1(C)c2ccccc2-c2c(-c3ccc4c(c3)B(c3ccccc3)OC4)cc(N(c3cccc(-n4c5ccccc5c5ccccc54)c3)c3ccc4ccccc4c3)cc21]

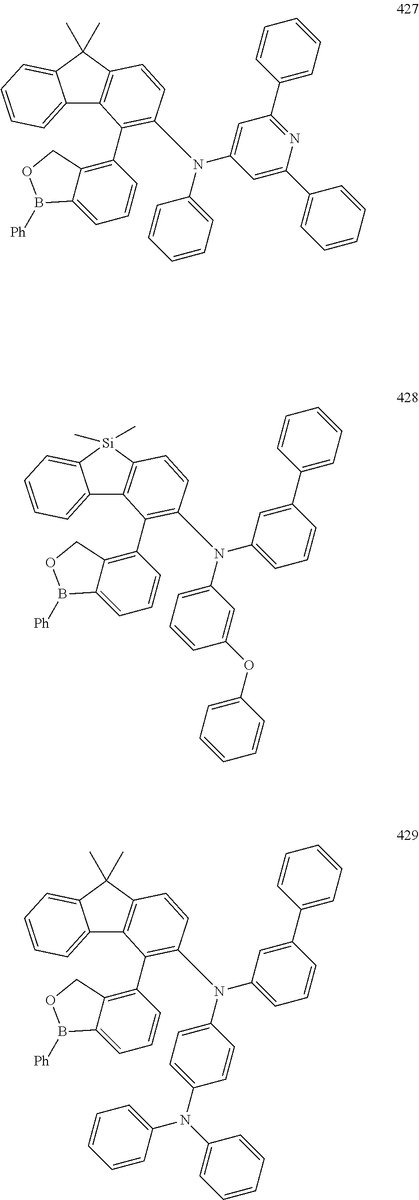 [SMILES: CC1(C)c2ccccc2-c2c1ccc(N(c1ccccc1)c1cc(-c3ccccc3)nc(-c3ccccc3)c1)c2-c1cccc2c1COB2c1ccccc1 ; C[Si]1(C)c2ccccc2-c2c1ccc(N(c1cccc(Oc3ccccc3)c1)c1cccc(-c3ccccc3)c1)c2-c1cccc2c1COB2c1ccccc1 ; CC1(C)c2ccccc2-c2c1ccc(N(c1ccc(N(c3ccccc3)c3ccccc3)cc1)c1cccc(-c3ccccc3)c1)c2-c1cccc2c1COB2c1ccccc1]

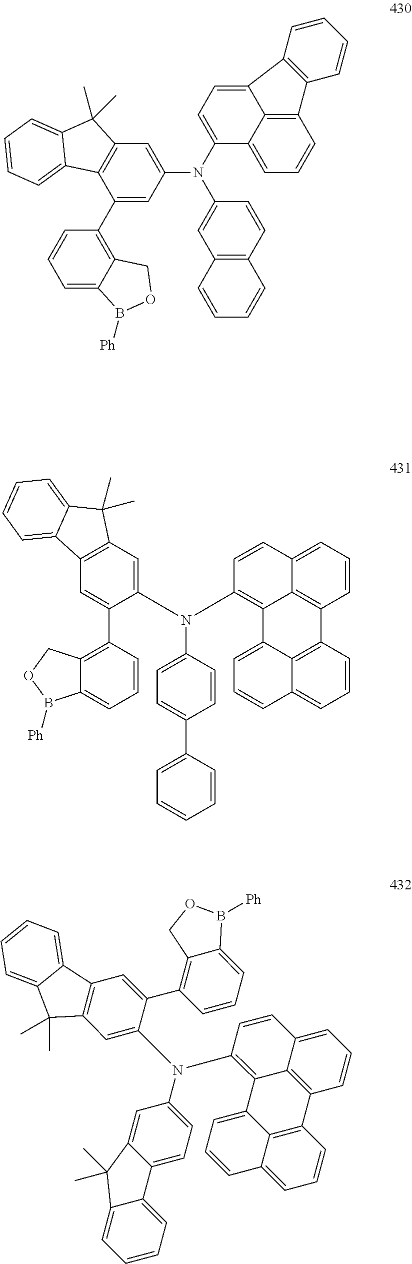 [SMILES: CC1(C)c2ccccc2-c2c(-c3cccc4c3COB4c3ccccc3)cc(N(c3ccc4ccccc4c3)c3ccc4c5c(cccc35)-c3ccccc3-4)cc21 ; CC1(C)c2ccccc2-c2cc(-c3cccc4c3COB4c3ccccc3)c(N(c3ccc(-c4ccccc4)cc3)c3ccc4cccc5c6cccc7cccc(c3c45)c76)cc21 ; CC1(C)c2ccccc2-c2ccc(N(c3cc4c(cc3-c3cccc5c3COB5c3ccccc3)-c3ccccc3C4(C)C)c3ccc4cccc5c6cccc7cccc(c3c45)c76)cc21]

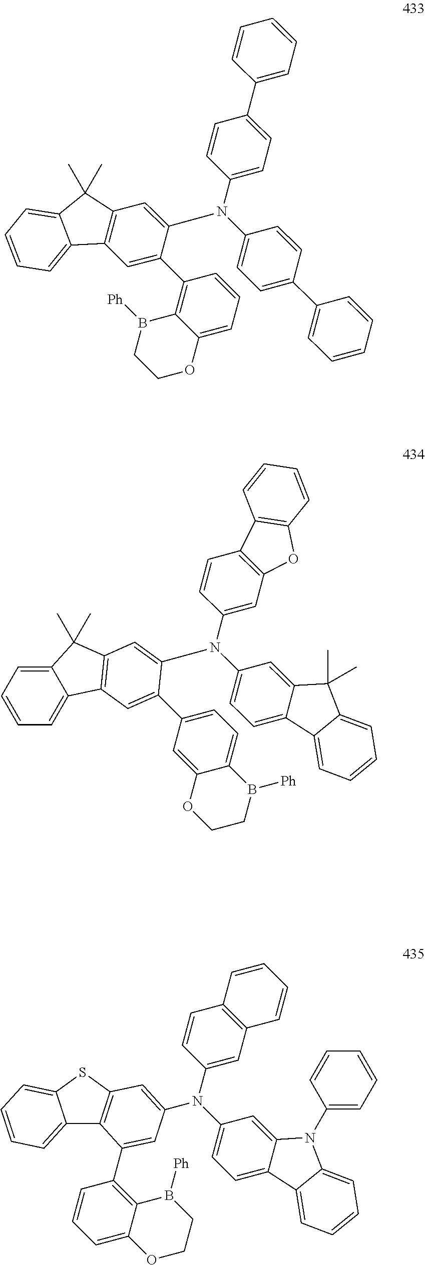 [SMILES: CC1(C)c2ccccc2-c2cc(-c3cccc4c3B(c3ccccc3)CCO4)c(N(c3ccc(-c4ccccc4)cc3)c3ccc(-c4ccccc4)cc3)cc21 ; CC1(C)c2ccccc2-c2ccc(N(c3ccc4c(c3)oc3ccccc34)c3cc4c(cc3-c3ccc5c(c3)OCCB5c3ccccc3)-c3ccccc3C4(C)C)cc21 ; c1ccc(B2CCOc3cccc(-c4cc(N(c5ccc6ccccc6c5)c5ccc6c7ccccc7n(-c7ccccc7)c6c5)cc5sc6ccccc6c45)c32)cc1]

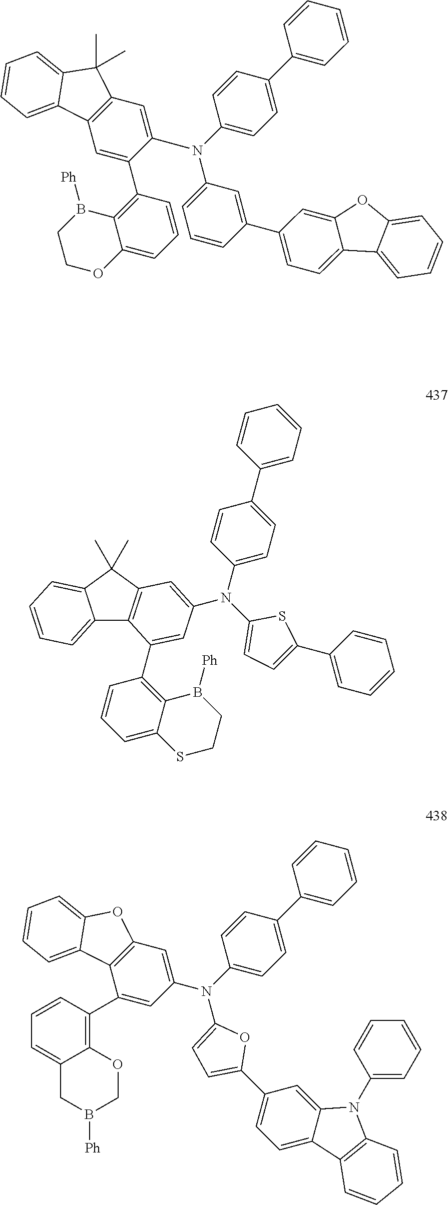 [SMILES: CC1(C)c2ccccc2-c2cc(-c3cccc4c3B(c3ccccc3)CCO4)c(N(c3ccc(-c4ccccc4)cc3)c3cccc(-c4ccc5c(c4)oc4ccccc45)c3)cc21 ; CC1(C)c2ccccc2-c2c(-c3cccc4c3B(c3ccccc3)CCS4)cc(N(c3ccc(-c4ccccc4)cc3)c3ccc(-c4ccccc4)s3)cc21 ; c1ccc(B2COc3c(cccc3-c3cc(N(c4ccc(-c5ccccc5)cc4)c4ccc(-c5ccc6c7ccccc7n(-c7ccccc7)c6c5)o4)cc4oc5ccccc5c34)C2)cc1]

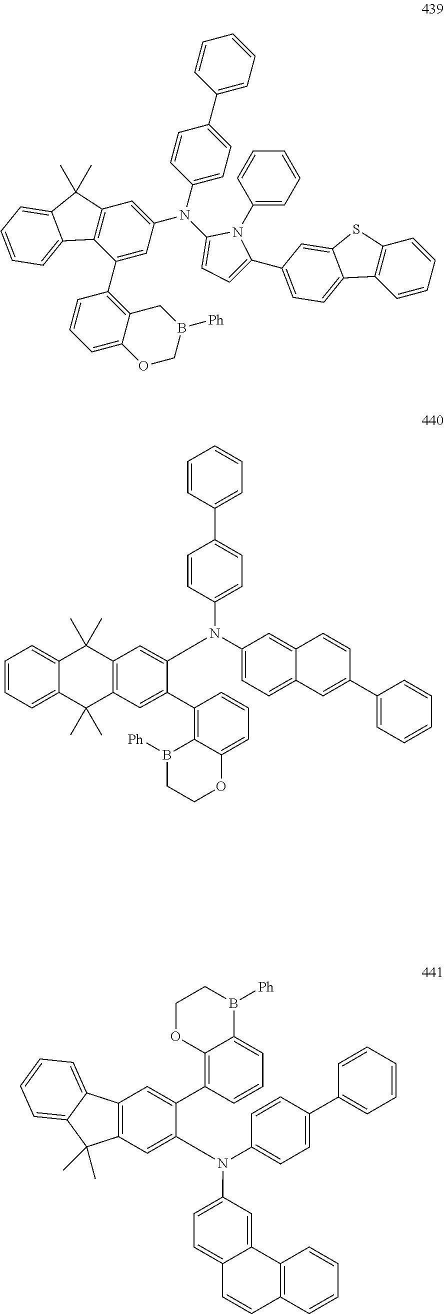 [SMILES: CC1(C)c2ccccc2-c2c(-c3cccc4c3CB(c3ccccc3)CO4)cc(N(c3ccc(-c4ccccc4)cc3)c3ccc(-c4ccc5c(c4)sc4ccccc45)n3-c3ccccc3)cc21 ; CC1(C)c2ccccc2C(C)(C)c2cc(N(c3ccc(-c4ccccc4)cc3)c3ccc4cc(-c5ccccc5)ccc4c3)c(-c3cccc4c3B(c3ccccc3)CCO4)cc21 ; CC1(C)c2ccccc2-c2cc(-c3cccc4c3OCCB4c3ccccc3)c(N(c3ccc(-c4ccccc4)cc3)c3ccc4ccc5ccccc5c4c3)cc21]

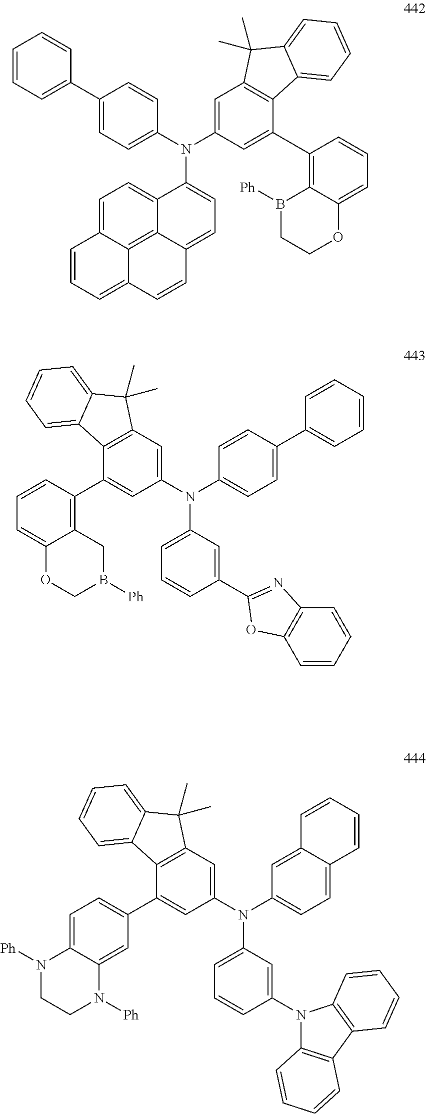 [SMILES: CC1(C)c2ccccc2-c2c(-c3cccc4c3B(c3ccccc3)CCO4)cc(N(c3ccc(-c4ccccc4)cc3)c3ccc4ccc5cccc6ccc3c4c56)cc21 ; CC1(C)c2ccccc2-c2c(-c3cccc4c3CB(c3ccccc3)CO4)cc(N(c3ccc(-c4ccccc4)cc3)c3cccc(-c4nc5ccccc5o4)c3)cc21 ; CC1(C)c2ccccc2-c2c(-c3ccc4c(c3)N(c3ccccc3)CCN4c3ccccc3)cc(N(c3cccc(-n4c5ccccc5c5ccccc54)c3)c3ccc4ccccc4c3)cc21]

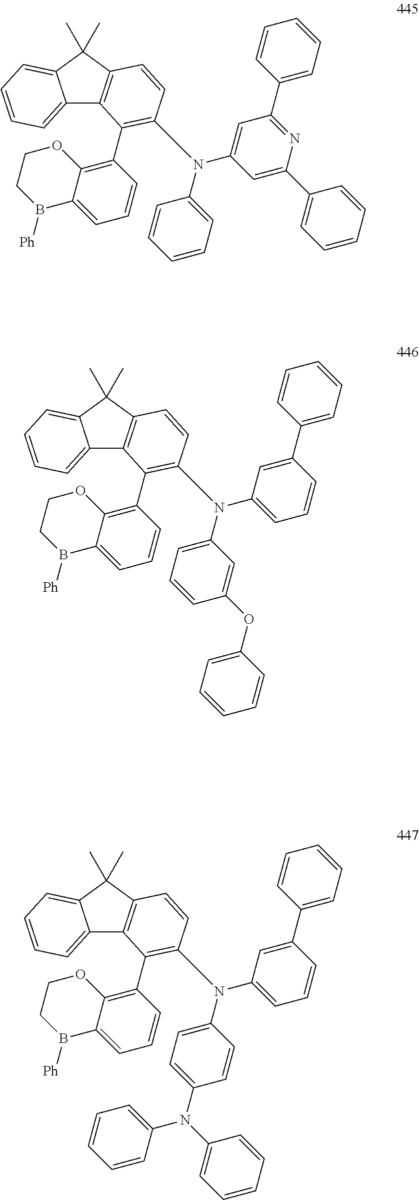 [SMILES: CC1(C)c2ccccc2-c2c1ccc(N(c1ccccc1)c1cc(-c3ccccc3)nc(-c3ccccc3)c1)c2-c1cccc2c1OCCB2c1ccccc1 ; CC1(C)c2ccccc2-c2c1ccc(N(c1cccc(Oc3ccccc3)c1)c1cccc(-c3ccccc3)c1)c2-c1cccc2c1OCCB2c1ccccc1 ; CC1(C)c2ccccc2-c2c1ccc(N(c1ccc(N(c3ccccc3)c3ccccc3)cc1)c1cccc(-c3ccccc3)c1)c2-c1cccc2c1OCCB2c1ccccc1]

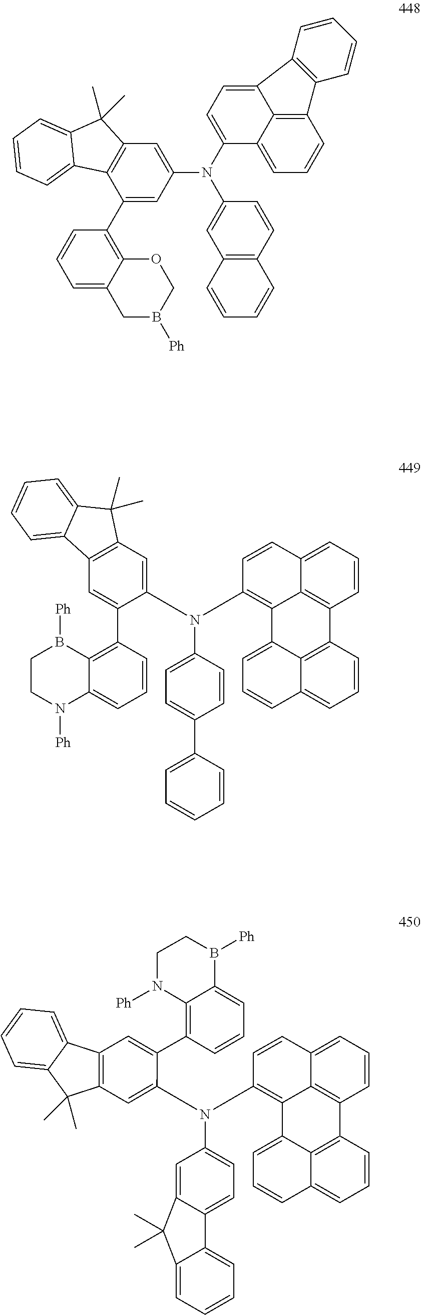 [SMILES: CC1(C)c2ccccc2-c2c(-c3cccc4c3OCB(c3ccccc3)C4)cc(N(c3ccc4ccccc4c3)c3ccc4c5c(cccc35)-c3ccccc3-4)cc21 ; CC1(C)c2ccccc2-c2cc(-c3cccc4c3B(c3ccccc3)CCN4c3ccccc3)c(N(c3ccc(-c4ccccc4)cc3)c3ccc4cccc5c6cccc7cccc(c3c45)c76)cc21 ; CC1(C)c2ccccc2-c2ccc(N(c3cc4c(cc3-c3cccc5c3N(c3ccccc3)CCB5c3ccccc3)-c3ccccc3C4(C)C)c3ccc4cccc5c6cccc7cccc(c3c45)c76)cc21]

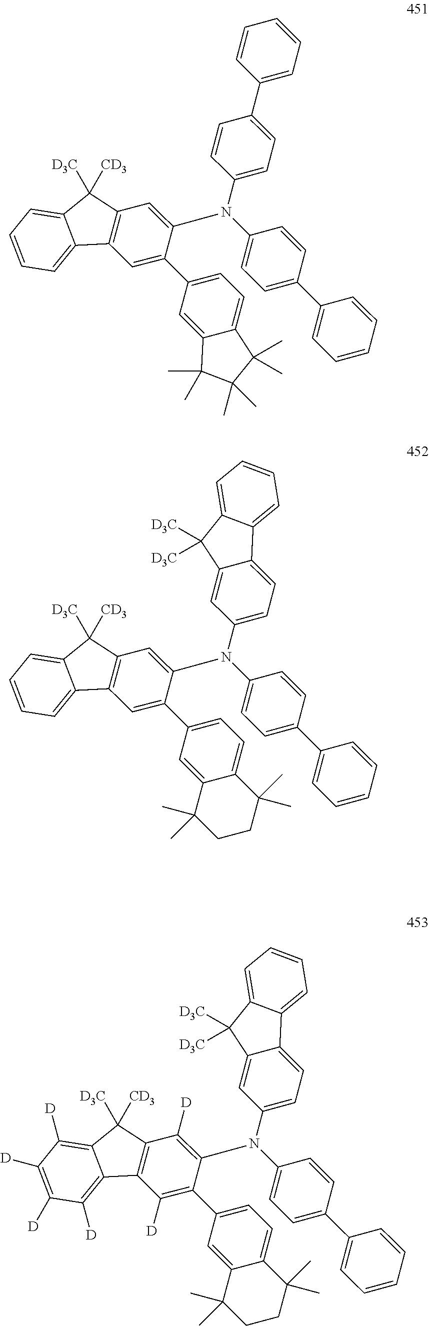

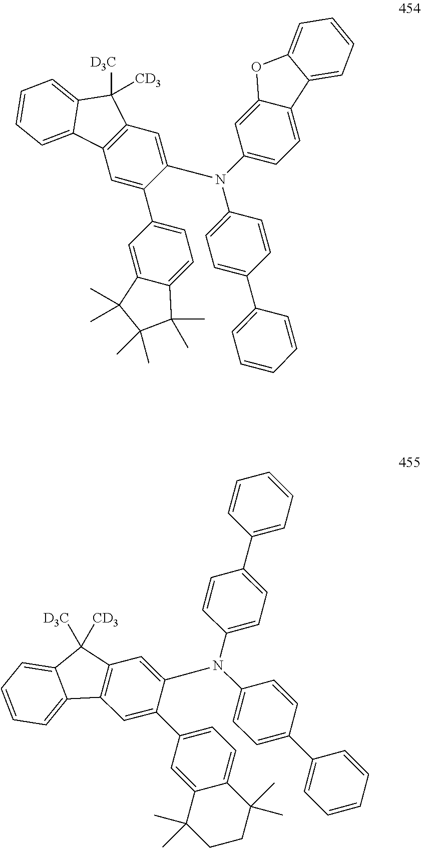 [SMILES: [2H]C([2H])([2H])C1(C([2H])([2H])[2H])c2ccccc2-c2cc(-c3ccc4c(c3)C(C)(C)C(C)(C)C4(C)C)c(N(c3ccc(-c4ccccc4)cc3)c3ccc4c(c3)oc3ccccc34)cc21 ; [2H]C([2H])([2H])C1(C([2H])([2H])[2H])c2ccccc2-c2cc(-c3ccc4c(c3)C(C)(C)CCC4(C)C)c(N(c3ccc(-c4ccccc4)cc3)c3ccc(-c4ccccc4)cc3)cc21]

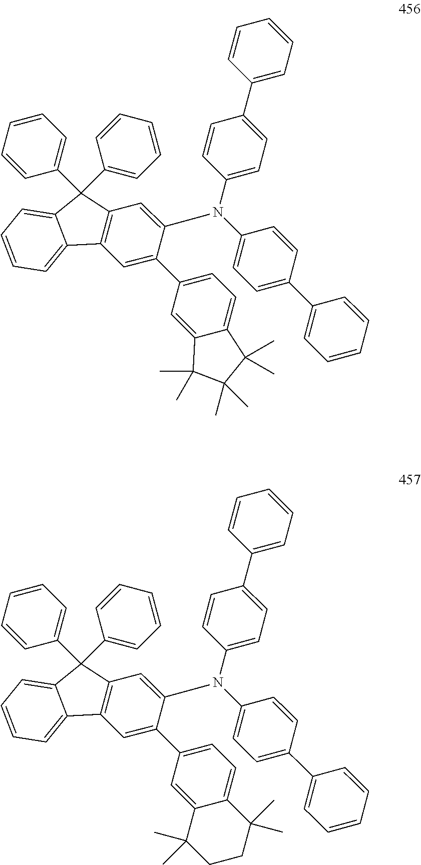 [SMILES: CC1(C)c2ccc(-c3cc4c(cc3N(c3ccc(-c5ccccc5)cc3)c3ccc(-c5ccccc5)cc3)C(c3ccccc3)(c3ccccc3)c3ccccc3-4)cc2C(C)(C)C1(C)C ; CC1(C)CCC(C)(C)c2cc(-c3cc4c(cc3N(c3ccc(-c5ccccc5)cc3)c3ccc(-c5ccccc5)cc3)C(c3ccccc3)(c3ccccc3)c3ccccc3-4)ccc21]

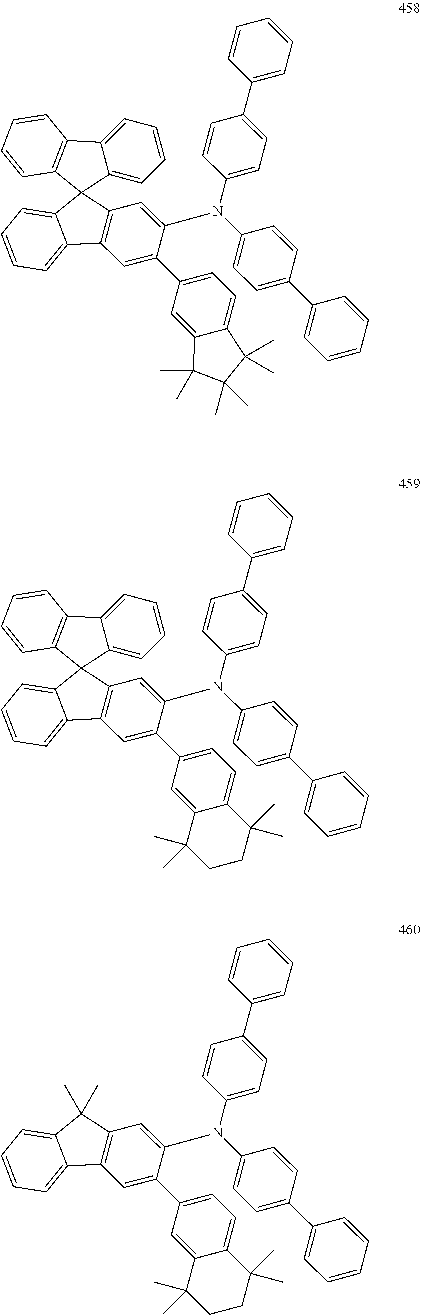 [SMILES: CC1(C)c2ccc(-c3cc4c(cc3N(c3ccc(-c5ccccc5)cc3)c3ccc(-c5ccccc5)cc3)C3(c5ccccc5-c5ccccc53)c3ccccc3-4)cc2C(C)(C)C1(C)C ; CC1(C)CCC(C)(C)c2cc(-c3cc4c(cc3N(c3ccc(-c5ccccc5)cc3)c3ccc(-c5ccccc5)cc3)C3(c5ccccc5-c5ccccc53)c3ccccc3-4)ccc21 ; CC1(C)CCC(C)(C)c2cc(-c3cc4c(cc3N(c3ccc(-c5ccccc5)cc3)c3ccc(-c5ccccc5)cc3)C(C)(C)c3ccccc3-4)ccc21]

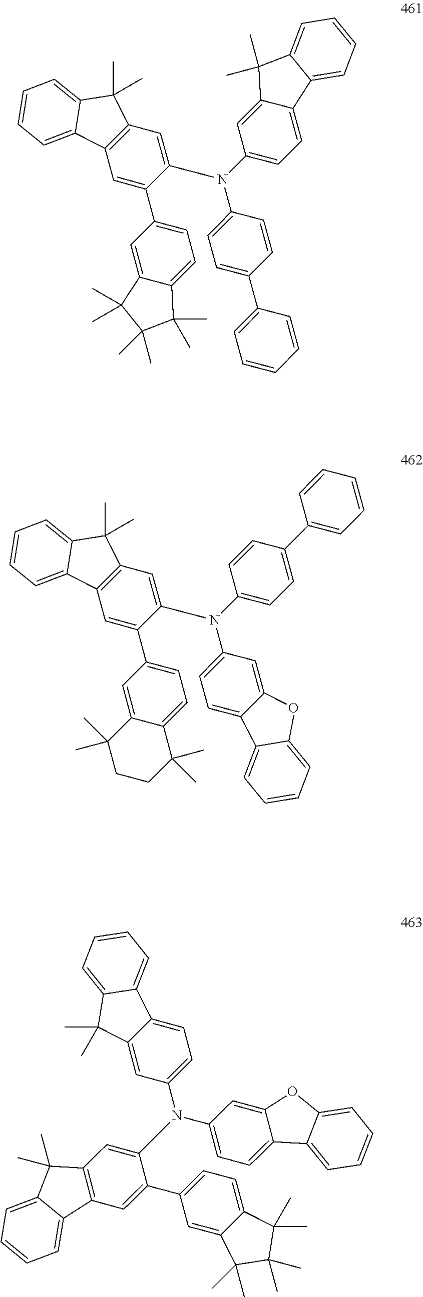 [SMILES: CC1(C)c2ccccc2-c2ccc(N(c3ccc(-c4ccccc4)cc3)c3cc4c(cc3-c3ccc5c(c3)C(C)(C)C(C)(C)C5(C)C)-c3ccccc3C4(C)C)cc21 ; CC1(C)CCC(C)(C)c2cc(-c3cc4c(cc3N(c3ccc(-c5ccccc5)cc3)c3ccc5c(c3)oc3ccccc35)C(C)(C)c3ccccc3-4)ccc21 ; CC1(C)c2ccccc2-c2ccc(N(c3ccc4c(c3)oc3ccccc34)c3cc4c(cc3-c3ccc5c(c3)C(C)(C)C(C)(C)C5(C)C)-c3ccccc3C4(C)C)cc21]

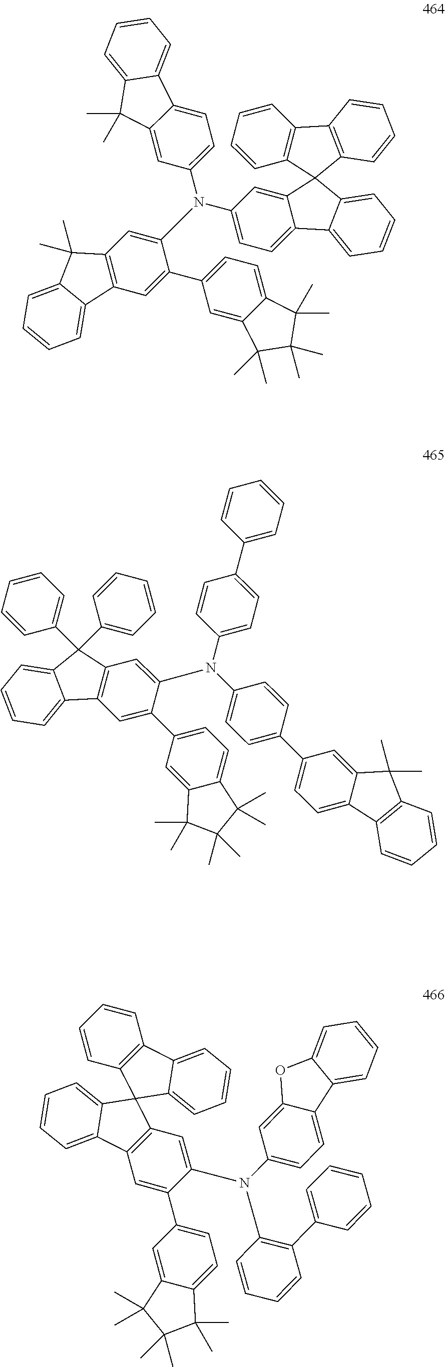 [SMILES: CC1(C)c2ccccc2-c2ccc(N(c3ccc4c(c3)C3(c5ccccc5-c5ccccc53)c3ccccc3-4)c3cc4c(cc3-c3ccc5c(c3)C(C)(C)C(C)(C)C5(C)C)-c3ccccc3C4(C)C)cc21 ; CC1(C)c2ccccc2-c2ccc(-c3ccc(N(c4ccc(-c5ccccc5)cc4)c4cc5c(cc4-c4ccc6c(c4)C(C)(C)C(C)(C)C6(C)C)-c4ccccc4C5(c4ccccc4)c4ccccc4)cc3)cc21 ; CC1(C)c2ccc(-c3cc4c(cc3N(c3ccc5c(c3)oc3ccccc35)c3ccccc3-c3ccccc3)C3(c5ccccc5-c5ccccc53)c3ccccc3-4)cc2C(C)(C)C1(C)C]

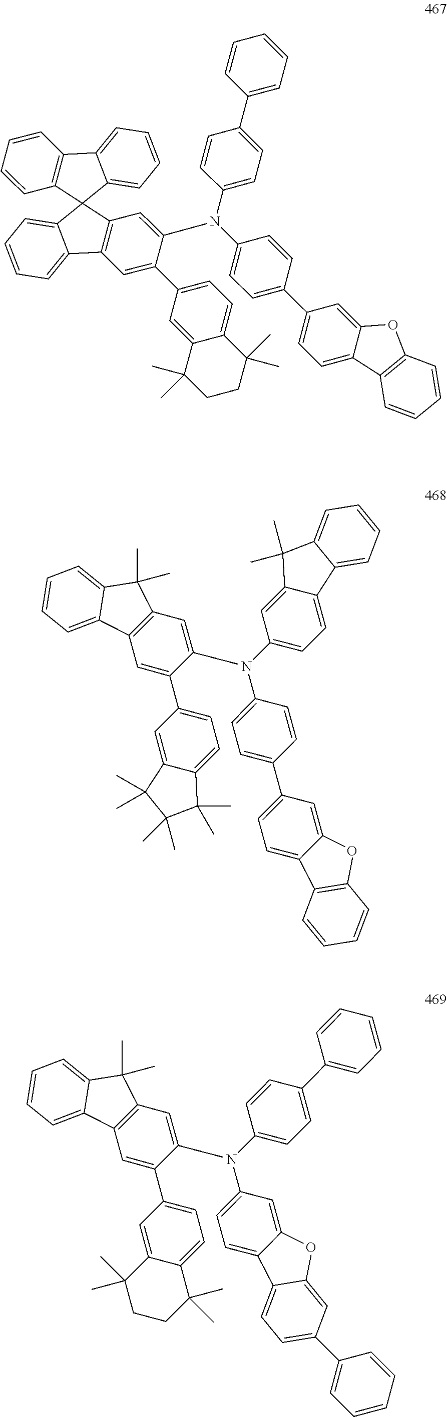 [SMILES: CC1(C)CCC(C)(C)c2cc(-c3cc4c(cc3N(c3ccc(-c5ccccc5)cc3)c3ccc(-c5ccc6c(c5)oc5ccccc56)cc3)C3(c5ccccc5-c5ccccc53)c3ccccc3-4)ccc21 ; CC1(C)c2ccccc2-c2ccc(N(c3ccc(-c4ccc5c(c4)oc4ccccc45)cc3)c3cc4c(cc3-c3ccc5c(c3)C(C)(C)C(C)(C)C5(C)C)-c3ccccc3C4(C)C)cc21 ; CC1(C)CCC(C)(C)c2cc(-c3cc4c(cc3N(c3ccc(-c5ccccc5)cc3)c3ccc5c(c3)oc3cc(-c6ccccc6)ccc35)C(C)(C)c3ccccc3-4)ccc21]

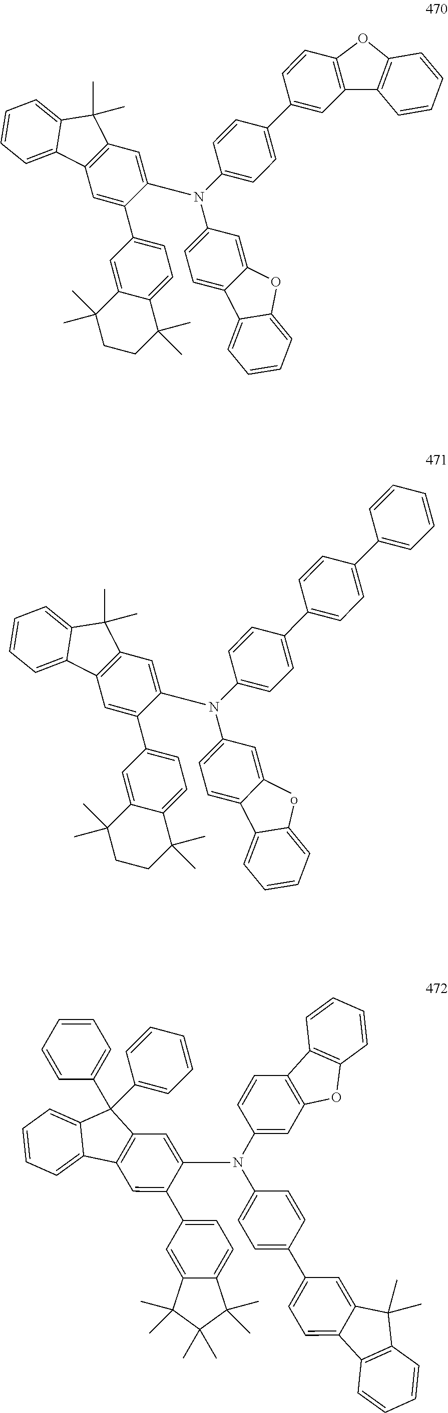 [SMILES: CC1(C)CCC(C)(C)c2cc(-c3cc4c(cc3N(c3ccc(-c5ccc6oc7ccccc7c6c5)cc3)c3ccc5c(c3)oc3ccccc35)C(C)(C)c3ccccc3-4)ccc21 ; CC1(C)CCC(C)(C)c2cc(-c3cc4c(cc3N(c3ccc(-c5ccc(-c6ccccc6)cc5)cc3)c3ccc5c(c3)oc3ccccc35)C(C)(C)c3ccccc3-4)ccc21 ; CC1(C)c2ccccc2-c2ccc(-c3ccc(N(c4ccc5c(c4)oc4ccccc45)c4cc5c(cc4-c4ccc6c(c4)C(C)(C)C(C)(C)C6(C)C)-c4ccccc4C5(c4ccccc4)c4ccccc4)cc3)cc21]

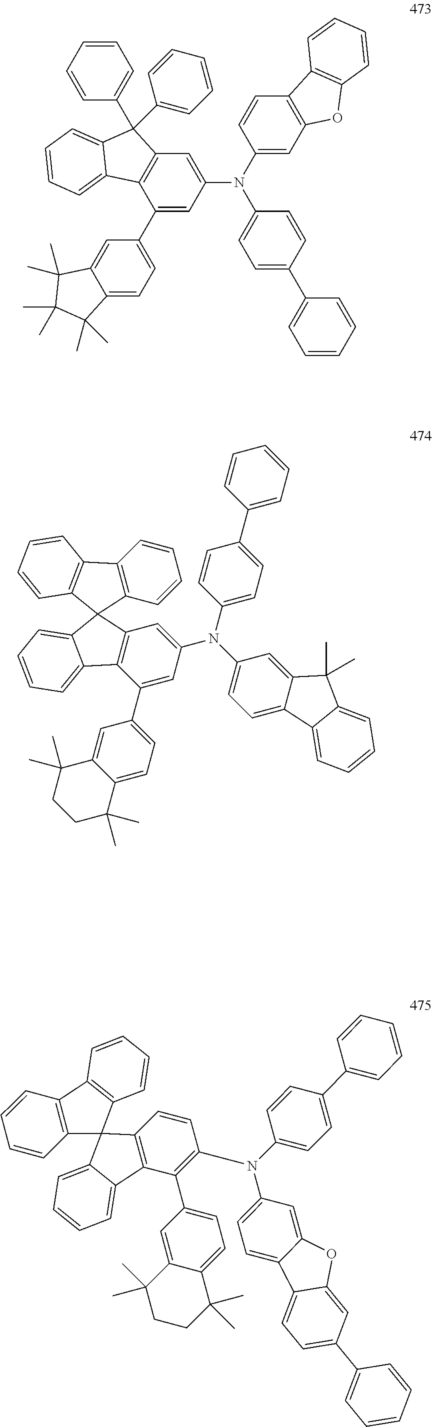 [SMILES: CC1(C)c2ccc(-c3cc(N(c4ccc(-c5ccccc5)cc4)c4ccc5c(c4)oc4ccccc45)cc4c3-c3ccccc3C4(c3ccccc3)c3ccccc3)cc2C(C)(C)C1(C)C ; CC1(C)CCC(C)(C)c2cc(-c3cc(N(c4ccc(-c5ccccc5)cc4)c4ccc5c(c4)C(C)(C)c4ccccc4-5)cc4c3-c3ccccc3C43c4ccccc4-c4ccccc43)ccc21 ; CC1(C)CCC(C)(C)c2cc(-c3c(N(c4ccc(-c5ccccc5)cc4)c4ccc5c(c4)oc4cc(-c6ccccc6)ccc45)ccc4c3-c3ccccc3C43c4ccccc4-c4ccccc43)ccc21]

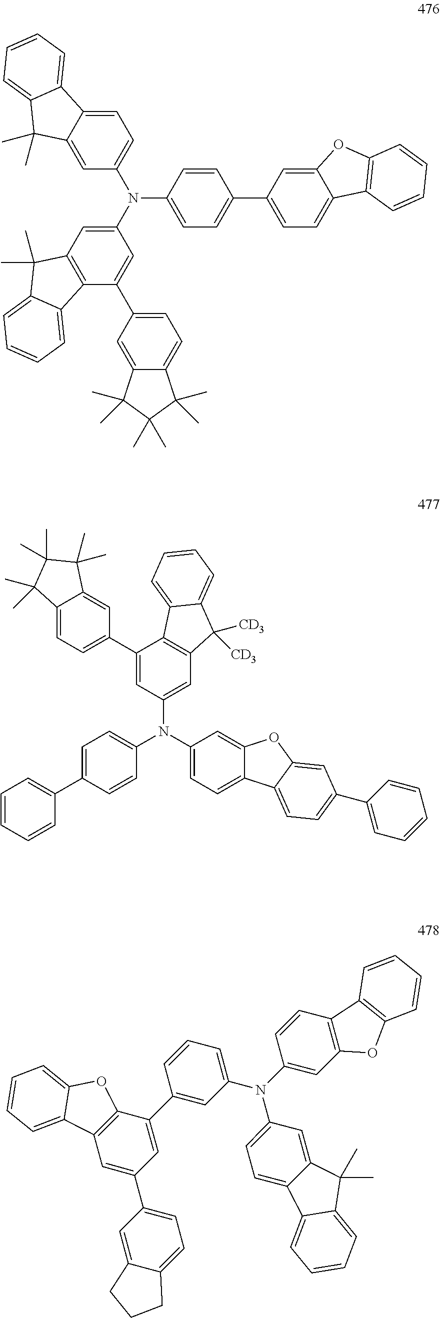 [SMILES: CC1(C)c2ccccc2-c2ccc(N(c3ccc(-c4ccc5c(c4)oc4ccccc45)cc3)c3cc(-c4ccc5c(c4)C(C)(C)C(C)(C)C5(C)C)c4c(c3)C(C)(C)c3ccccc3-4)cc21 ; [2H]C([2H])([2H])C1(C([2H])([2H])[2H])c2ccccc2-c2c(-c3ccc4c(c3)C(C)(C)C(C)(C)C4(C)C)cc(N(c3ccc(-c4ccccc4)cc3)c3ccc4c(c3)oc3cc(-c5ccccc5)ccc34)cc21 ; CC1(C)c2ccccc2-c2ccc(N(c3cccc(-c4cc(-c5ccc6c(c5)CCC6)cc5c4oc4ccccc45)c3)c3ccc4c(c3)oc3ccccc34)cc21]

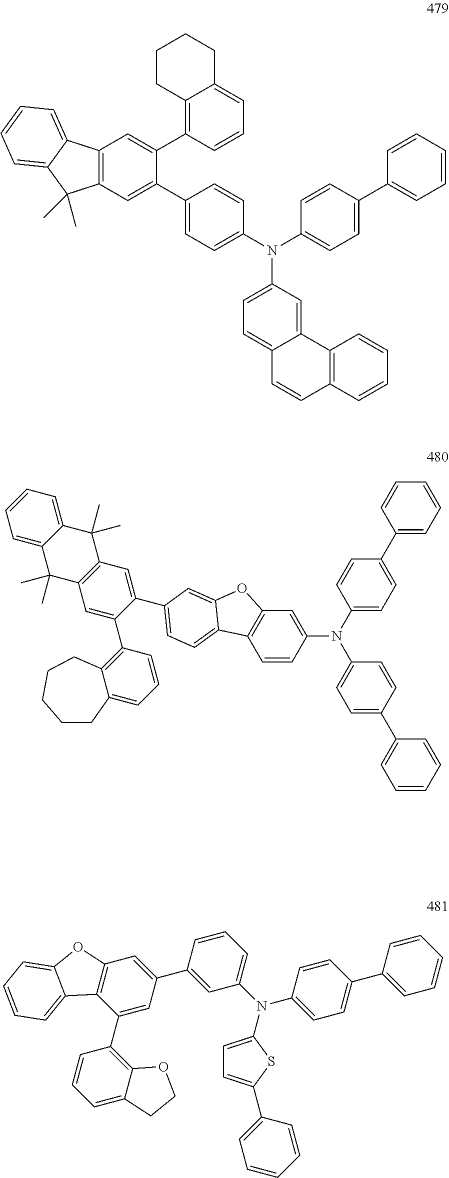 [SMILES: CC1(C)c2ccccc2-c2cc(-c3cccc4c3CCCC4)c(-c3ccc(N(c4ccc(-c5ccccc5)cc4)c4ccc5ccc6ccccc6c5c4)cc3)cc21 ; CC1(C)c2ccccc2C(C)(C)c2cc(-c3cccc4c3CCCCC4)c(-c3ccc4c(c3)oc3cc(N(c5ccc(-c6ccccc6)cc5)c5ccc(-c6ccccc6)cc5)ccc34)cc21 ; c1ccc(-c2ccc(N(c3cccc(-c4cc(-c5cccc6c5OCC6)c5c(c4)oc4ccccc45)c3)c3ccc(-c4ccccc4)s3)cc2)cc1]

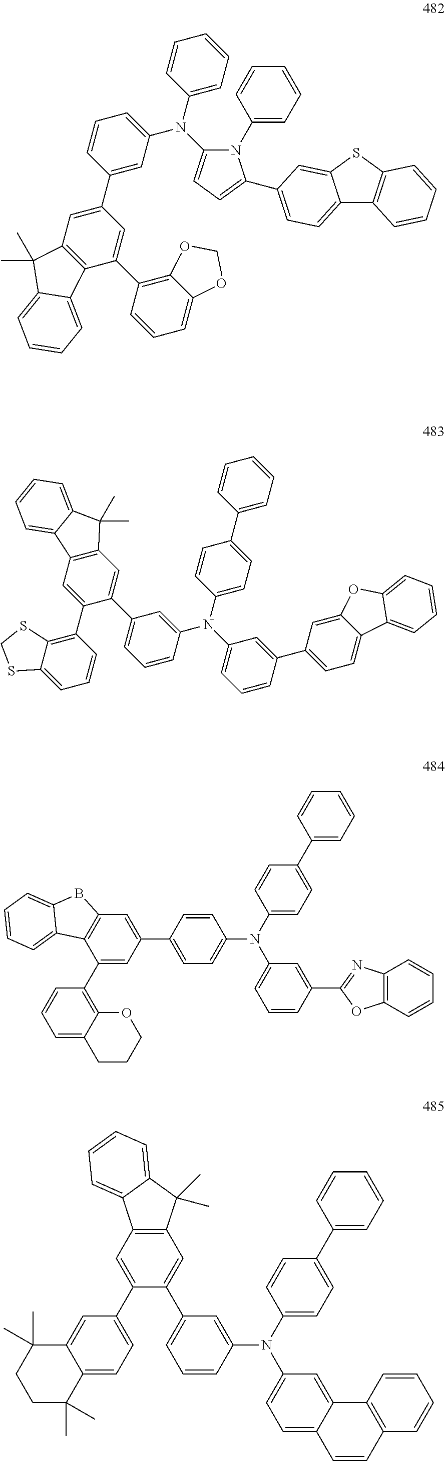

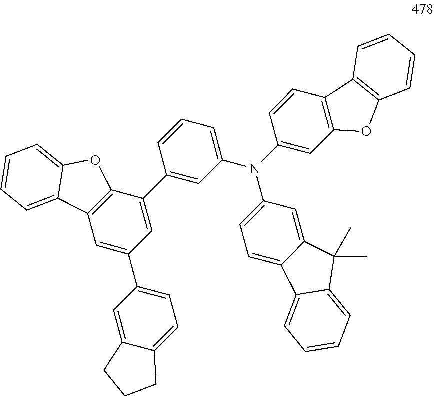 [SMILES: CC1(C)c2ccccc2-c2ccc(N(c3cccc(-c4cc(-c5ccc6c(c5)CCC6)cc5c4oc4ccccc45)c3)c3ccc4c(c3)oc3ccccc34)cc21]

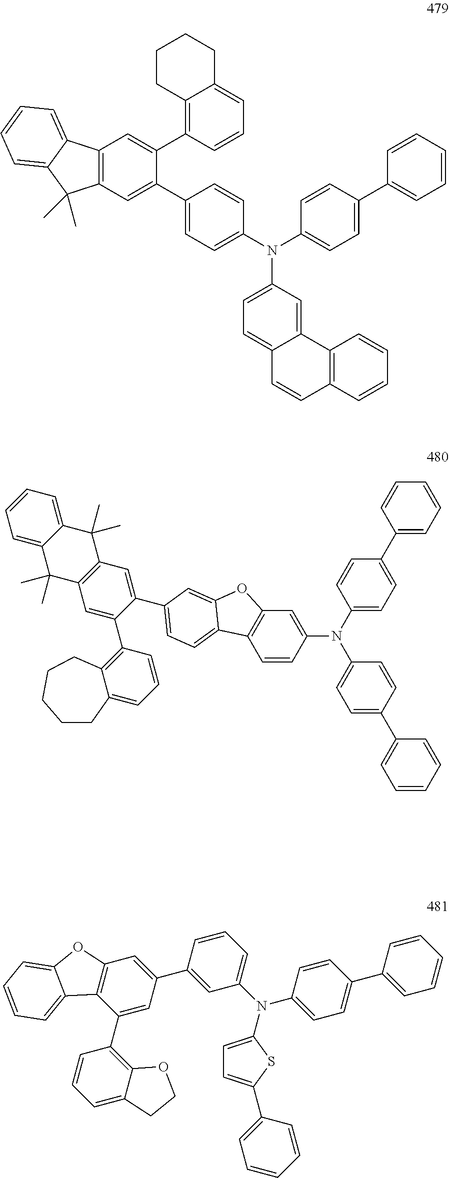 [SMILES: CC1(C)c2ccccc2-c2cc(-c3cccc4c3CCCC4)c(-c3ccc(N(c4ccc(-c5ccccc5)cc4)c4ccc5ccc6ccccc6c5c4)cc3)cc21 ; CC1(C)c2ccccc2C(C)(C)c2cc(-c3cccc4c3CCCCC4)c(-c3ccc4c(c3)oc3cc(N(c5ccc(-c6ccccc6)cc5)c5ccc(-c6ccccc6)cc5)ccc34)cc21 ; c1ccc(-c2ccc(N(c3cccc(-c4cc(-c5cccc6c5OCC6)c5c(c4)oc4ccccc45)c3)c3ccc(-c4ccccc4)s3)cc2)cc1]

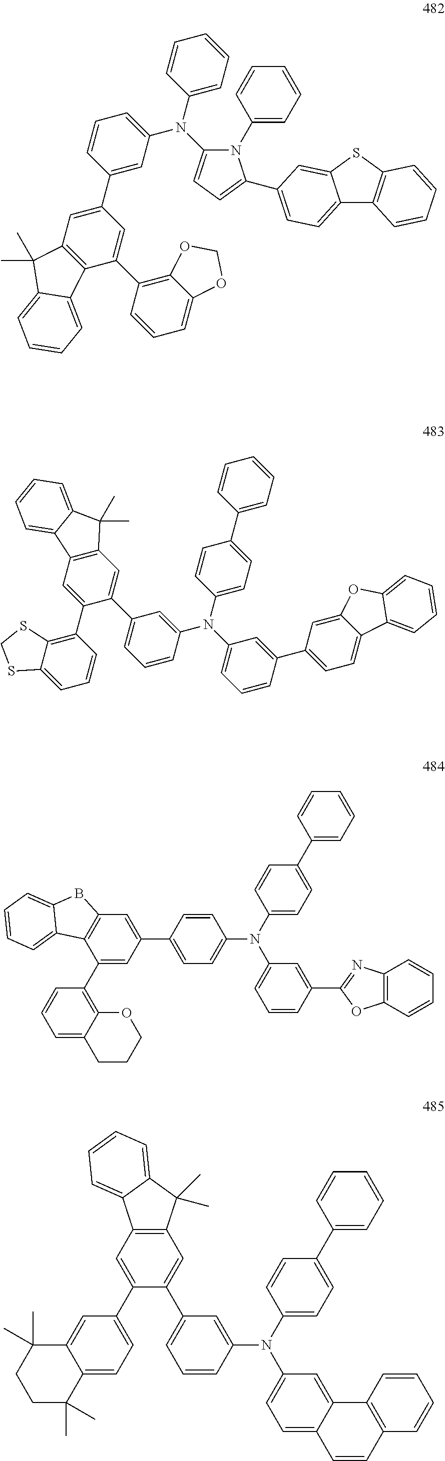

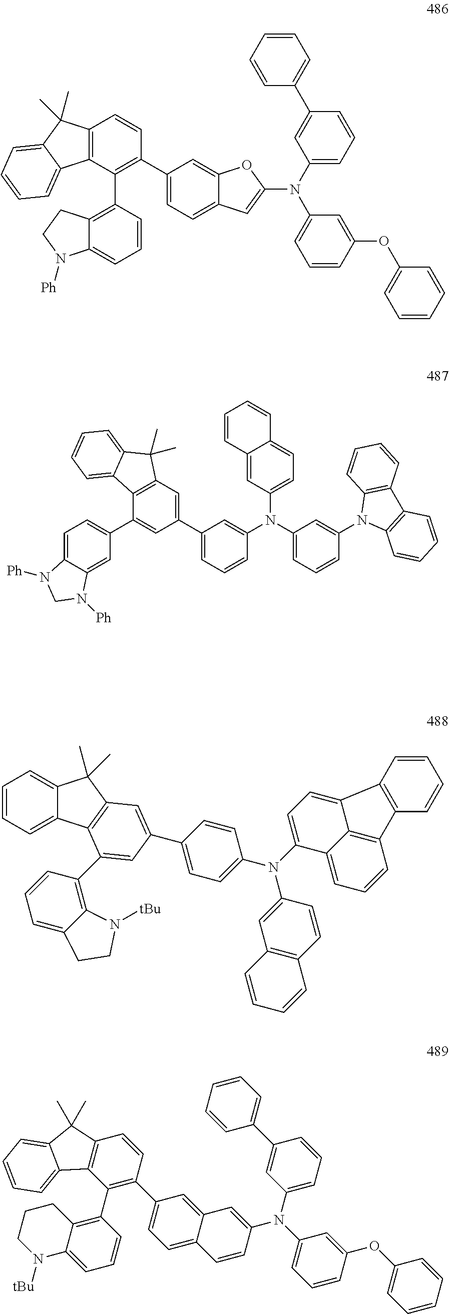 [SMILES: CC1(C)c2ccccc2-c2c1ccc(-c1ccc3cc(N(c4cccc(Oc5ccccc5)c4)c4cccc(-c5ccccc5)c4)oc3c1)c2-c1cccc2c1CCN2c1ccccc1 ; CC1(C)c2ccccc2-c2c(-c3ccc4c(c3)N(c3ccccc3)CN4c3ccccc3)cc(-c3cccc(N(c4cccc(-n5c6ccccc6c6ccccc65)c4)c4ccc5ccccc5c4)c3)cc21 ; CC1(C)c2ccccc2-c2c(-c3cccc4c3N(C(C)(C)C)CC4)cc(-c3ccc(N(c4ccc5ccccc5c4)c4ccc5c6c(cccc46)-c4ccccc4-5)cc3)cc21 ; CC1(C)c2ccccc2-c2c1ccc(-c1ccc3ccc(N(c4cccc(Oc5ccccc5)c4)c4cccc(-c5ccccc5)c4)cc3c1)c2-c1cccc2c1CCCN2C(C)(C)C]

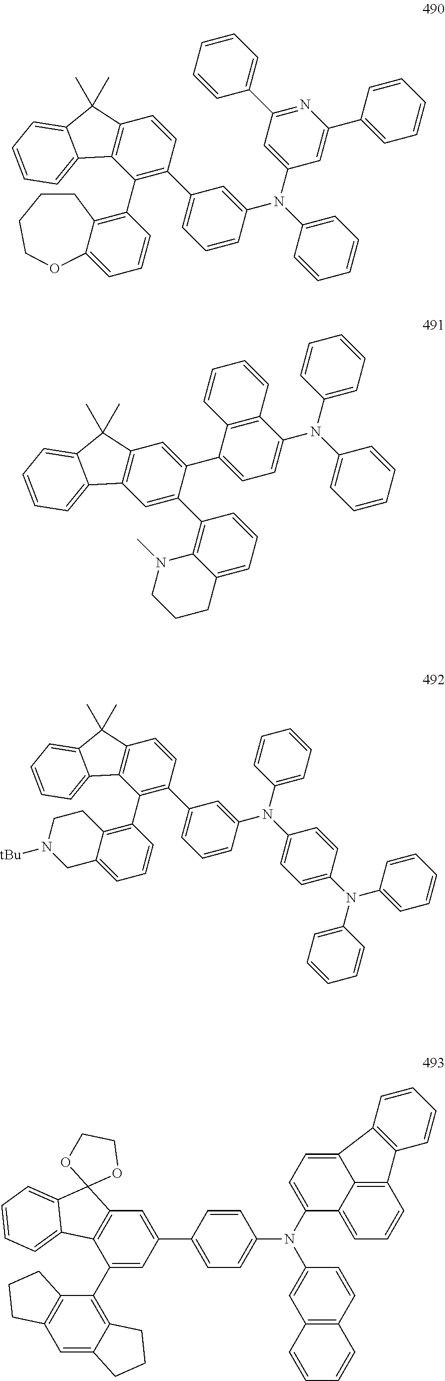 [SMILES: CC1(C)c2ccccc2-c2c1ccc(-c1cccc(N(c3ccccc3)c3cc(-c4ccccc4)nc(-c4ccccc4)c3)c1)c2-c1cccc2c1CCCCO2 ; CN1CCCc2cccc(-c3cc4c(cc3-c3ccc(N(c5ccccc5)c5ccccc5)c5ccccc35)C(C)(C)c3ccccc3-4)c21 ; CC1(C)c2ccccc2-c2c1ccc(-c1cccc(N(c3ccccc3)c3ccc(N(c4ccccc4)c4ccccc4)cc3)c1)c2-c1cccc2c1CCN(C(C)(C)C)C2 ; c1ccc2c(c1)-c1cccc3c(N(c4ccc(-c5cc(-c6c7c(cc8c6CCC8)CCC7)c6c(c5)C5(OCCO5)c5ccccc5-6)cc4)c4ccc5ccccc5c4)ccc-2c13]

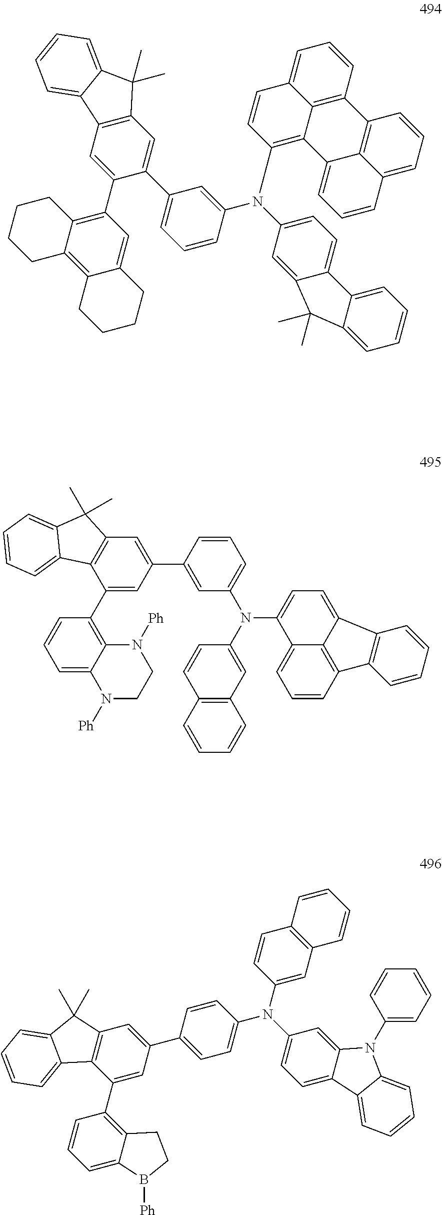 [SMILES: CC1(C)c2ccccc2-c2ccc(N(c3cccc(-c4cc5c(cc4-c4cc6c(c7c4CCCC7)CCCC6)-c4ccccc4C5(C)C)c3)c3ccc4cccc5c6cccc7cccc(c3c45)c76)cc21 ; CC1(C)c2ccccc2-c2c(-c3cccc4c3N(c3ccccc3)CCN4c3ccccc3)cc(-c3cccc(N(c4ccc5ccccc5c4)c4ccc5c6c(cccc46)-c4ccccc4-5)c3)cc21 ; CC1(C)c2ccccc2-c2c(-c3cccc4c3CCB4c3ccccc3)cc(-c3ccc(N(c4ccc5ccccc5c4)c4ccc5c6ccccc6n(-c6ccccc6)c5c4)cc3)cc21]

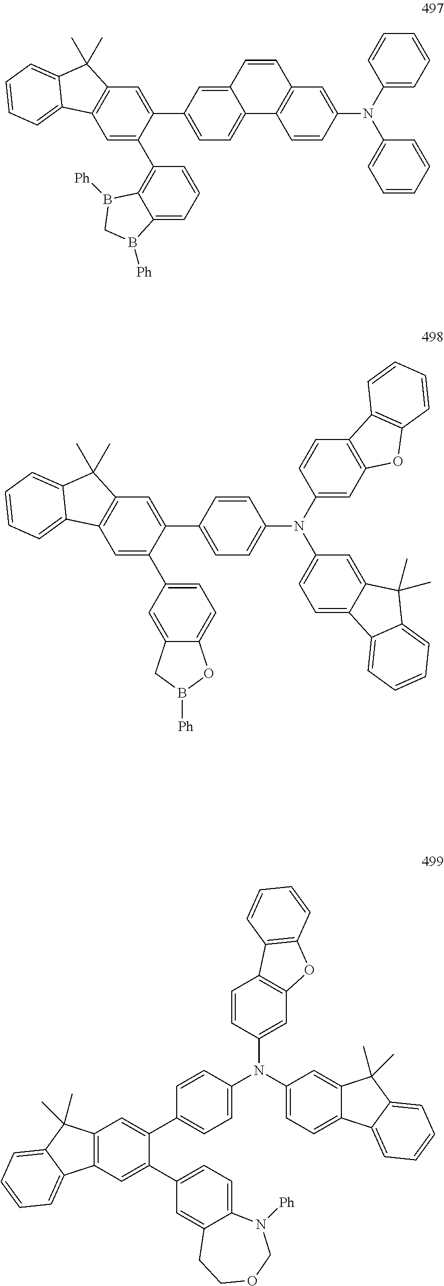 [SMILES: CC1(C)c2ccccc2-c2cc(-c3cccc4c3B(c3ccccc3)CB4c3ccccc3)c(-c3ccc4c(ccc5cc(N(c6ccccc6)c6ccccc6)ccc54)c3)cc21 ; CC1(C)c2ccccc2-c2ccc(N(c3ccc(-c4cc5c(cc4-c4ccc6c(c4)CB(c4ccccc4)O6)-c4ccccc4C5(C)C)cc3)c3ccc4c(c3)oc3ccccc34)cc21 ; CC1(C)c2ccccc2-c2ccc(N(c3ccc(-c4cc5c(cc4-c4ccc6c(c4)CCOCN6c4ccccc4)-c4ccccc4C5(C)C)cc3)c3ccc4c(c3)oc3ccccc34)cc21]

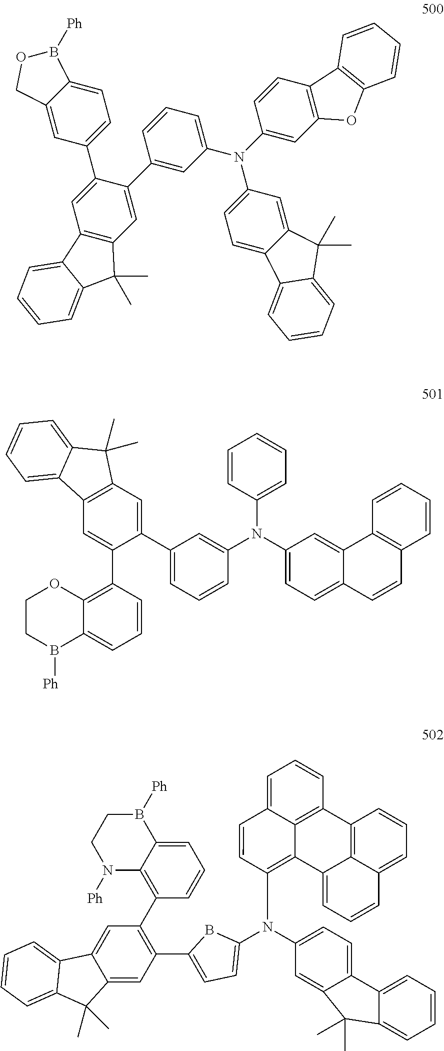

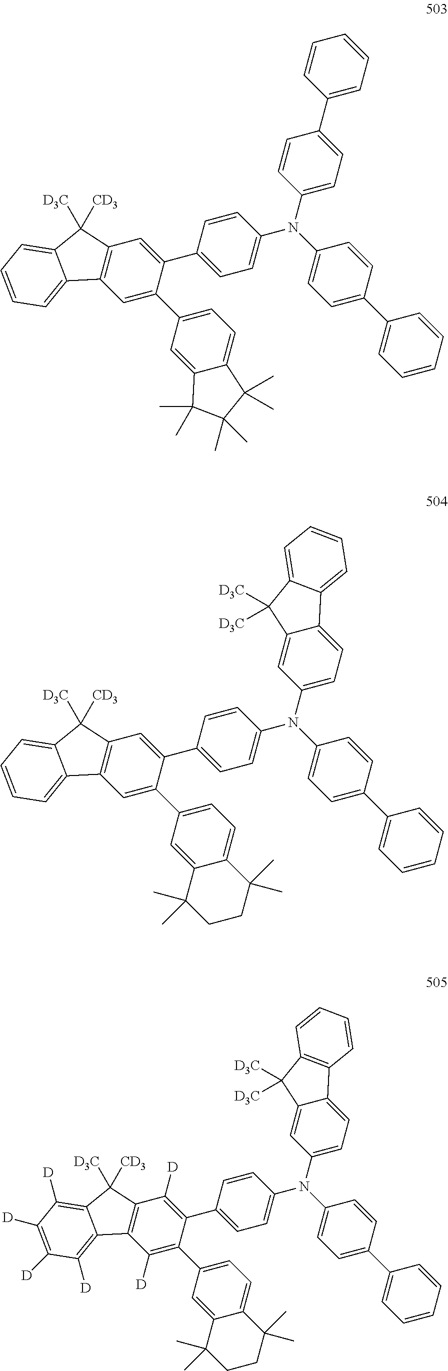

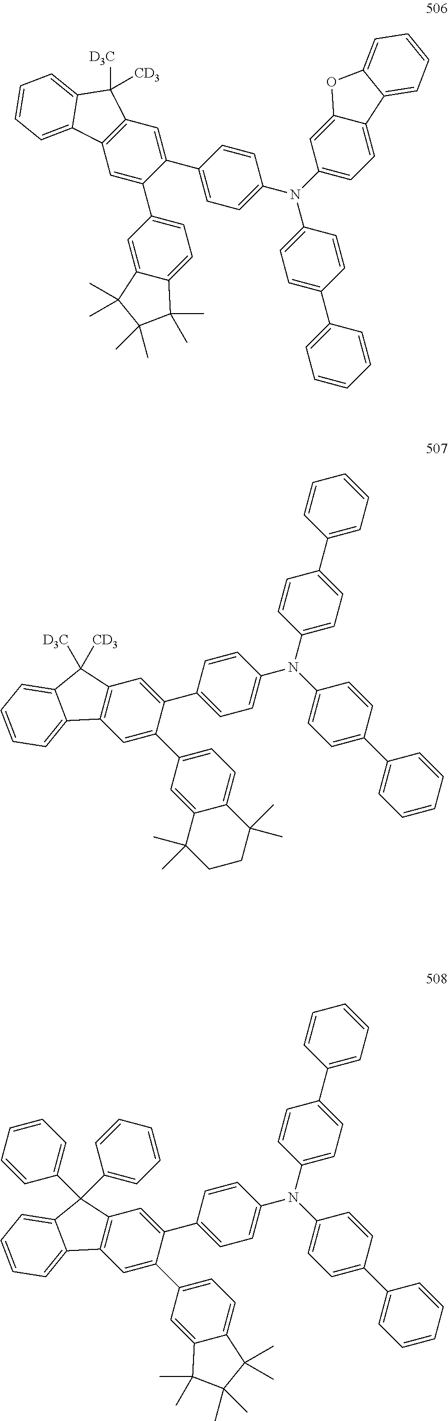 [SMILES: [2H]C([2H])([2H])C1(C([2H])([2H])[2H])c2ccccc2-c2cc(-c3ccc4c(c3)C(C)(C)C(C)(C)C4(C)C)c(-c3ccc(N(c4ccc(-c5ccccc5)cc4)c4ccc5c(c4)oc4ccccc45)cc3)cc21 ; [2H]C([2H])([2H])C1(C([2H])([2H])[2H])c2ccccc2-c2cc(-c3ccc4c(c3)C(C)(C)CCC4(C)C)c(-c3ccc(N(c4ccc(-c5ccccc5)cc4)c4ccc(-c5ccccc5)cc4)cc3)cc21 ; CC1(C)c2ccc(-c3cc4c(cc3-c3ccc(N(c5ccc(-c6ccccc6)cc5)c5ccc(-c6ccccc6)cc5)cc3)C(c3ccccc3)(c3ccccc3)c3ccccc3-4)cc2C(C)(C)C1(C)C]

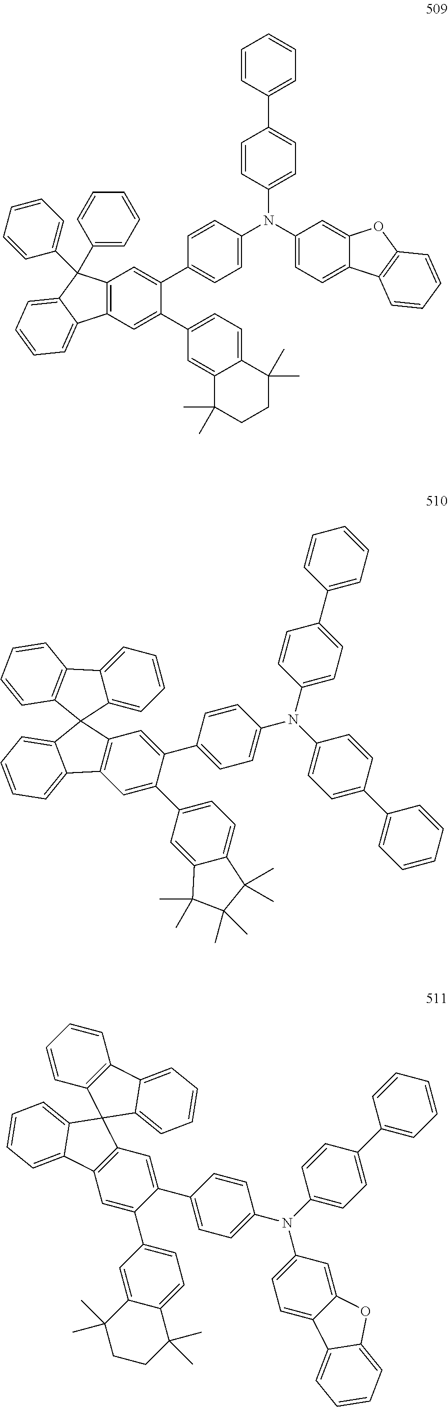 [SMILES: CC1(C)CCC(C)(C)c2cc(-c3cc4c(cc3-c3ccc(N(c5ccc(-c6ccccc6)cc5)c5ccc6c(c5)oc5ccccc56)cc3)C(c3ccccc3)(c3ccccc3)c3ccccc3-4)ccc21 ; CC1(C)c2ccc(-c3cc4c(cc3-c3ccc(N(c5ccc(-c6ccccc6)cc5)c5ccc(-c6ccccc6)cc5)cc3)C3(c5ccccc5-c5ccccc53)c3ccccc3-4)cc2C(C)(C)C1(C)C ; CC1(C)CCC(C)(C)c2cc(-c3cc4c(cc3-c3ccc(N(c5ccc(-c6ccccc6)cc5)c5ccc6c(c5)oc5ccccc56)cc3)C3(c5ccccc5-c5ccccc53)c3ccccc3-4)ccc21]

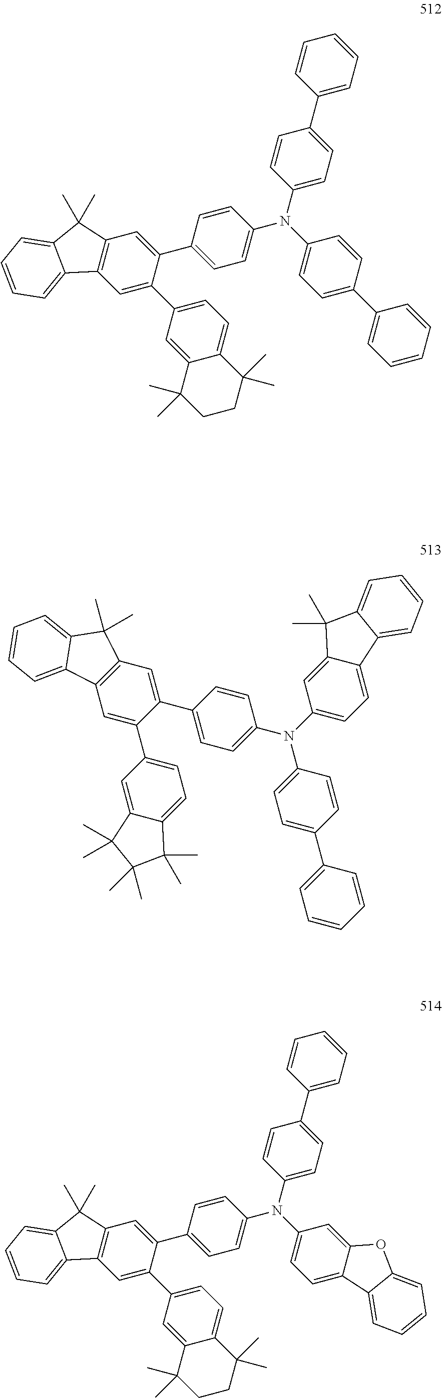 [SMILES: CC1(C)CCC(C)(C)c2cc(-c3cc4c(cc3-c3ccc(N(c5ccc(-c6ccccc6)cc5)c5ccc(-c6ccccc6)cc5)cc3)C(C)(C)c3ccccc3-4)ccc21 ; CC1(C)c2ccccc2-c2ccc(N(c3ccc(-c4ccccc4)cc3)c3ccc(-c4cc5c(cc4-c4ccc6c(c4)C(C)(C)C(C)(C)C6(C)C)-c4ccccc4C5(C)C)cc3)cc21 ; CC1(C)CCC(C)(C)c2cc(-c3cc4c(cc3-c3ccc(N(c5ccc(-c6ccccc6)cc5)c5ccc6c(c5)oc5ccccc56)cc3)C(C)(C)c3ccccc3-4)ccc21]

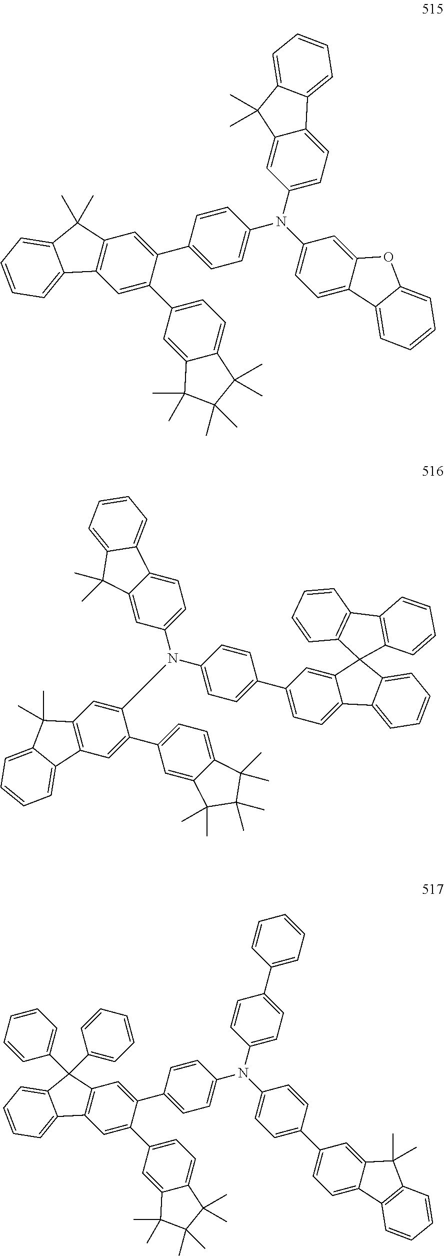 [SMILES: CC1(C)c2ccccc2-c2ccc(N(c3ccc(-c4cc5c(cc4-c4ccc6c(c4)C(C)(C)C(C)(C)C6(C)C)-c4ccccc4C5(C)C)cc3)c3ccc4c(c3)oc3ccccc34)cc21 ; CC1(C)c2ccccc2-c2ccc(N(c3ccc(-c4ccc5c(c4)C4(c6ccccc6-c6ccccc64)c4ccccc4-5)cc3)c3cc4c(cc3-c3ccc5c(c3)C(C)(C)C(C)(C)C5(C)C)-c3ccccc3C4(C)C)cc21 ; CC1(C)c2ccccc2-c2ccc(-c3ccc(N(c4ccc(-c5ccccc5)cc4)c4ccc(-c5cc6c(cc5-c5ccc7c(c5)C(C)(C)C(C)(C)C7(C)C)-c5ccccc5C6(c5ccccc5)c5ccccc5)cc4)cc3)cc21]

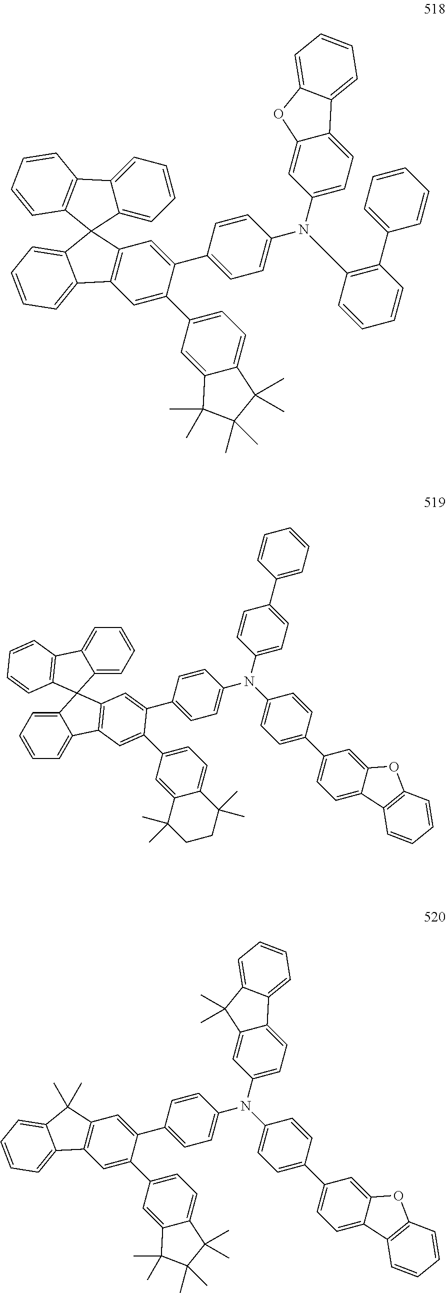 [SMILES: CC1(C)c2ccc(-c3cc4c(cc3-c3ccc(N(c5ccc6c(c5)oc5ccccc56)c5ccccc5-c5ccccc5)cc3)C3(c5ccccc5-c5ccccc53)c3ccccc3-4)cc2C(C)(C)C1(C)C ; CC1(C)CCC(C)(C)c2cc(-c3cc4c(cc3-c3ccc(N(c5ccc(-c6ccccc6)cc5)c5ccc(-c6ccc7c(c6)oc6ccccc67)cc5)cc3)C3(c5ccccc5-c5ccccc53)c3ccccc3-4)ccc21 ; CC1(C)c2ccccc2-c2ccc(N(c3ccc(-c4ccc5c(c4)oc4ccccc45)cc3)c3ccc(-c4cc5c(cc4-c4ccc6c(c4)C(C)(C)C(C)(C)C6(C)C)-c4ccccc4C5(C)C)cc3)cc21]

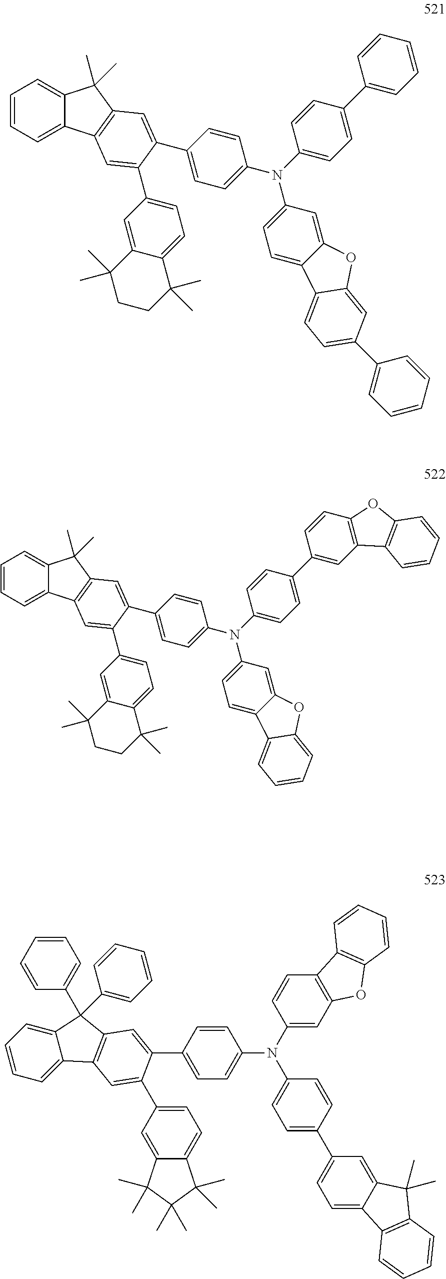 [SMILES: CC1(C)CCC(C)(C)c2cc(-c3cc4c(cc3-c3ccc(N(c5ccc(-c6ccccc6)cc5)c5ccc6c(c5)oc5cc(-c7ccccc7)ccc56)cc3)C(C)(C)c3ccccc3-4)ccc21 ; CC1(C)CCC(C)(C)c2cc(-c3cc4c(cc3-c3ccc(N(c5ccc(-c6ccc7oc8ccccc8c7c6)cc5)c5ccc6c(c5)oc5ccccc56)cc3)C(C)(C)c3ccccc3-4)ccc21 ; CC1(C)c2ccccc2-c2ccc(-c3ccc(N(c4ccc(-c5cc6c(cc5-c5ccc7c(c5)C(C)(C)C(C)(C)C7(C)C)-c5ccccc5C6(c5ccccc5)c5ccccc5)cc4)c4ccc5c(c4)oc4ccccc45)cc3)cc21]

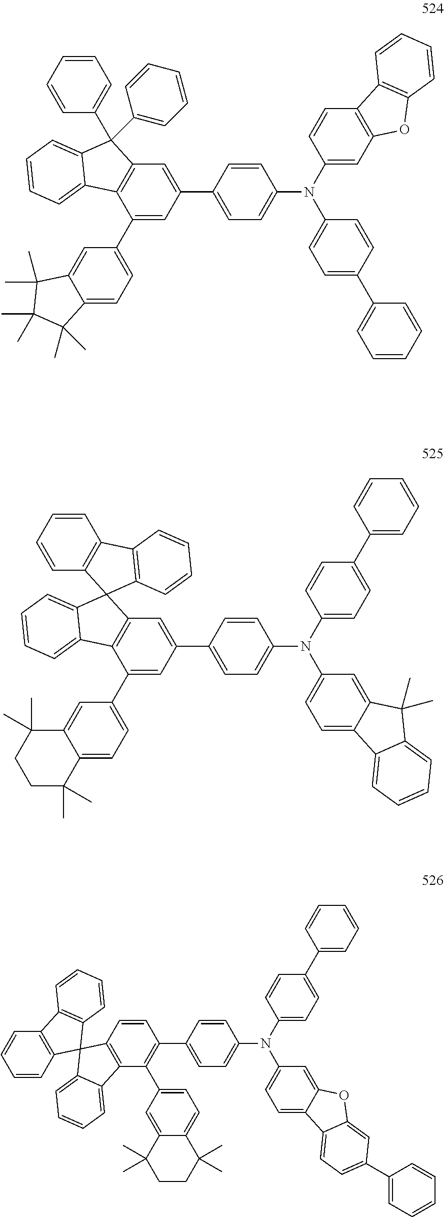

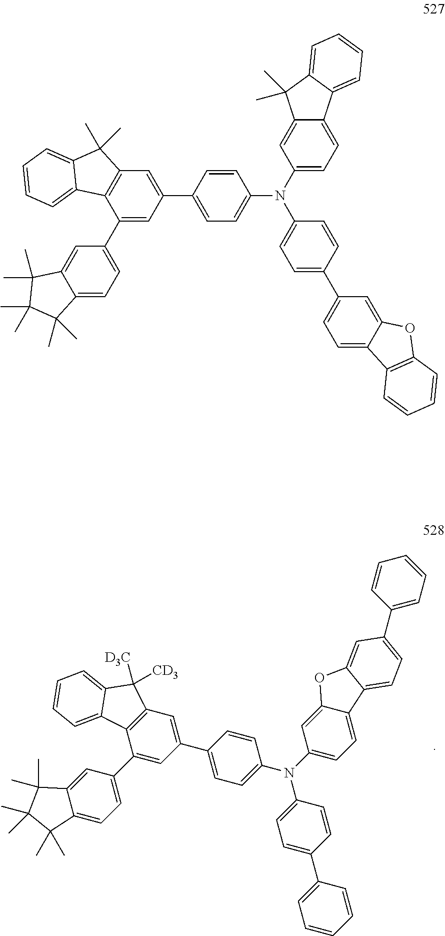 [SMILES: CC1(C)c2ccccc2-c2ccc(N(c3ccc(-c4cc(-c5ccc6c(c5)C(C)(C)C(C)(C)C6(C)C)c5c(c4)C(C)(C)c4ccccc4-5)cc3)c3ccc(-c4ccc5c(c4)oc4ccccc45)cc3)cc21 ; [2H]C([2H])([2H])C1(C([2H])([2H])[2H])c2ccccc2-c2c(-c3ccc4c(c3)C(C)(C)C(C)(C)C4(C)C)cc(-c3ccc(N(c4ccc(-c5ccccc5)cc4)c4ccc5c(c4)oc4cc(-c6ccccc6)ccc45)cc3)cc21]

Specifically, the above structure may be unsubstituted or substituted with one or more substituents selected from the following. For example, the substituent may be deuterium, a halogen group, a nitrile group, a nitro group, a hydroxyl group, a carbonyl group, an ester group, an imide group, an amine group, a phosphine oxide group, an alkoxy group, an aryloxy group, an alkylthio group, an arylthio group, an alkylsulfonyl group, an arylsulfonyl group, a silyl group, a boron group, an alkyl group, a cycloalkyl group, an alkenyl group, an aryl group, an aralkyl group, an aralkenyl group, an alkylaryl group, an alkylamine group, an aralkylamine group, a heteroarylamine group, an arylamine group, an arylheteroarylamine group, an arylphosphino group, a heteroaryl group, and the like.

The compounds of the present disclosure have different material lifetime depending on the different molecular structures and application scenarios. According to the specific conditions of group A and group B (as long as there is one non-carbon atom in group B, it is a heterocyclic structure), the enumerated structures may include the following categories:

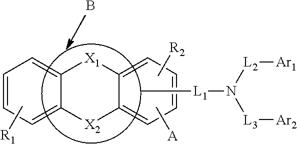

When X₁ in group B is arbitrarily selected from carbon atoms CR₉R₁₀, and X₂ does not contain heteroatoms, that is, a single bond or CR₉R₁₀, since neither the single bond nor CR₉R₁₀ has strong electron absorption or electron-dominating properties, the effect on the molecular properties is limited. When X₂ is arbitrarily selected from carbon atoms CR₉R₁₀, and X₁ does not contain heteroatoms, that is, a single bond or CR₉R₁₀, since neither the single bond nor CR₉R₁₀ has strong electron absorption or electron-dominating properties, the effect on the molecular properties is limited. These two situations may be classified into a same category. Among them, R₉° R₁₀, and R₉R₁₀ are defined in the same way. X₁, X₂ in group B are each independently selected from a single bond, CR₉R₁₀, NR₁₁, SR₁₂R₁₃, or S. As long as there is a heteroatom in group B (any one of NR₁₁, SiR₁₂R₁₃, O or S), it is classified as a heterocyclic structure, and its performance is greatly affected by the heteroatom and may be classified into one category. For example, when X₁ is selected from any heteroatom NR₁₁, SiR₁₂R₁₃, O or S, regardless of whether X₂ is a single bond, CR₉R₁₀ or X₂ is the heteroatom NR₁₁, SiR₁₂R₁₃, O or S, the performance is affected by the heteroatom of X₁. Similarly, when X₂ is selected from any heteroatom NR₁₁, SiR₁₂R₁₃, O or S, regardless of whether X₁ is a single bond, CR₉R₁₀, or it is the heteroatom NR₁₁, SiR₁₂R₁₃, O or S, the performance is affected by the heteroatom X₂. These two situations may be classified into one category. Since the heteroatom NR₁₁, SiR₁₂R₁₃, O or S have a large electronegativity difference, the effect of such electronegativity on the molecules is classified with decreasing in the electronegativity of elements, which varies at a large extent, so the impact on the device performance will vary. According to O, N, the situations are as follows:

| | B | | | |
|---|---|---|---|---|
| A | X₁(X₂) | X₂(X₁) | Type | Enumerated combination |
| CR₃R₄ | CR₉R₁₀ | Single bond or | 1 | 1, 4, 5, 7, 9, 10, 12, 13, 14, 15, 16, 17, 18, 19, 20, 21, 23, |
| | | carbon atom | | 24, 25, 26, 27, 29, 30, 31, 32, 33, 34, 35, 36, 37, 38, 39, 40, |
| | | group | | 41, 42, 45, 46, 47, 48, 49, 51, 52, 53, 54, 128, 129, 130, |
| | | | | 131, 132, 133, 135, 136, 137, 138, 139, 140, 141, 142, |
| | | | | 143, 144, 147, 171, 172, 274, 275, 276, 277, 278, 280, |
| | | | | 281, 282, 283, 284, 285, 287, 288, 289, 290, 291, 292, |
| | | | | 294, 295, 296, 297, 298, 299, 300, 301, 302, 303, 304, |
| | | | | 305, 306, 351, 451, 452, 453, 454, 456, 457, 458, 459, |
| | | | | 460, 461, 462, 463, 464, 465, 466, 467, 468, 469, 470, |
| | | | | 471, 472, 473, 474, 475, 476, 477, 478, 480, 485, 494, |
| | | | | 503, 504, 505, 506, 507, 508, 509, 510, 511, 512, 513, |
| | | | | 514, 515, 516, 517, 518, 519, 520, 521, 522, 523, 524, |
| | | | | 525, 526, 527, 528 |
| | NR₁₁ | N or S or C or | 2 | 3, 43, 127, 279 |
| | | single bond | | |
| | O | O or N or S or C | 3 | 2, 8, 11, 22, 28, 50, 134, 273, 286, 478, 481, 493 |
| | | or single bond | | |
| | S | S or C or single | 4 | 6, 44, 293 |
| | | bond | | |
| NR₅ | CR₉R₁₀ | Single bond or | 5 | 146, 147, 148, 149, 150, 151, 152, 153, 154, 155, 156, |
| | | carbon atom | | 157, 158, 159, 162, 170, 164, 165, 166, 167, 168, 169, |
| | | group | | 171, 172, 173, 174, 176, 177, 179, 180, 181, 182, 184, |
| | | | | 186, 187, 188, 189, 190, 191, 192, 193, 195, 196, 198, |
| | | | | 199, 200, 201, 202, 203, 205, 206, 207, 208, 209, 210, |
| | | | | 211, 212, 214, 215, 216, 235, 236, 237, 238, 239, 240, |
| | | | | 241, 242, 243, 244, 245, 246, 247, 248, 249, 250, 251, |
| | | | | 252, 253, 254, 255, 257, 258, 259, 260, 261, 262, 264, |
| | | | | 265, 266, 267, 270, 308, 309, 310, 311, 312, 313, 314, |
| | | | | 315, 316, 317, 318, 319, 320, 321, 322, 323, 324, 325, |
| | | | | 326, 327, 328, 329, 330, 331, 332, 333, 334, 335, 336, |
| | | | | 339, 340, 341, 342, 344, 365, 369, 372, 373, 374, 398, |
| | | | | 399, 400, 401, 402, 411, 412, 486, 487, 488, 489, 491, |
| | | | | 492, 495 |
| | NR₁₁ | N or S or C or | 6 | 183, 204, 307 |
| | | single bond | | |
| | O | O or N or S or C | 7 | 160, 163, 170, 185, 194, 256, 268, 337, 368, 370, 413 |
| | | or single bond | | |
| | S | S or C or single | 8 | 145, 161, 175, 178, 197, 263, 269 |
| | | bond | | |
| O | CR₉R₁₀ | Single bond or | 9 | 56, 58, 60, 61, 62, 63, 65, 66, 67, 69, 70, 71, 72, 73, 74, |
| | | carbon atom | | 76, 77, 79, 80, 81, 82, 83, 84, 85, 86, 87, 88, 90, 109, 110, |
| | | group | | 111, 112, 113, 114, 116, 117, 120, 122, 125, 126, 218, |
| | | | | 219, 221, 222, 226, 227, 228, 229, 230, 234, 365, 371, |
| | | | | 375, 376, 377, 378, 398, 409, 482, 490, 499 |
| | NR₁₁ | N or S or C or | 10 | 57 |
| | | single bond | | |
| | SiR₁₂R₁₃ | Si or C or single | 11 | 68 |
| | | bond | | |
| | O | O or N or S or C | 12 | 59, 78, 115, 217 |
| | | or single bond | | |
| | S | S or C or single | 13 | 55, 64, 75, 220, 366, 484 |
| | | bond | | |
| S | CR₉R₁₀ | Single bond or | 14 | 91, 92, 94, 95, 96, 97, 101, 102, 103, 104, 105, 106, 107, |
| | | carbon atom | | 108, 118, 121, 123, 223, 224, 225, 231, 232, 233, 483 |
| | | group | | |
| | O | O or N or S or C | 15 | 93, 98, 99, 100 |
| | | or single bond | | |
| BR₈ | CR₉R₁₀ | Single bond or | 16 | 343, 344, 345, 347, 349, 350, 351, 353, 354, 356, 357, |
| | | carbon atom | | 358, 360, 361, 379, 380, 381, 383, 384, 385, 386, 387, |
| | | group | | 389, 390, 391, 392, 393, 394, 395, 396, 397, 400, 5, |
| | | | | 406, 407, 408, 414, 416, 418, 420, 421, 422, 423, 425, |
| | | | | 426, 427, 429, 430, 431, 432, 433, 434, 436, 437, 439, |
| | | | | 440, 441, 442, 444, 445, 446, 447, 448, 449, 450, 496, |
| | | | | 497, 498, 500, 501, 502 |
| | NR₁₁ | N or S or C or | 17 | 352, 363, 403, 417 |
| | | single bond | | |
| | SiR₁₂R₁₃ | Si or C or single | 18 | 428 |
| | | bond | | |
| | O | O or N or S or C | 19 | 348, 359, 362, 382, 388, 419, 438 |
| | | or single bond | | |
| | S | S or C or single | 20 | 346, 355, 404, 415, 424, 435 |
| | | bond | | |

The compounds of the present disclosure may form an organic layer with other compounds, and applied in various scenarios. Such an organic layer may be applied in an organic optoelectronic device.

The compounds of the present disclosure have different triplet energy levels of the molecules due to their different compositions. According to different application scenarios, a suitable energy level may be selected for application in red light devices or green light devices.

The compounds of the present disclosure have different molecular properties due to different group combinations, and may be used in a green light buffer layer. The so-called green light buffer layer refers to a functional layer that can adjust the migration rate and number of electrons and holes in the device.

The organic optoelectronic device provided by the present disclosure includes a first electrode, a second electrode, and one or more organic layers arranged between the first electrode and the second electrode. It is in a bottom or top light-emitting structure. The organic layer may be a single-layer structure, or a multi-layer series structure with two or more organic layers laminated together. The organic layer includes at least one of a hole injection layer, a hole transport layer, a light-emitting layer, an electron injection layer or an electron transport layer. The conventional methods and materials for preparing organic optoelectronic devices may be used for preparation of this device. The organic optoelectronic device of the present disclosure uses the compound as the organic layer of the organic optoelectronic device.

In the organic optoelectronic device provided by the present disclosure, the first electrode serves as an anode layer, and the anode material may be, for example, a material with a large work function, so that holes are smoothly injected into the organic layer. More examples include metals, metal oxides, combinations of metals and oxides, conductive polymers, and the like. The metal oxide may be, for example, indium tin oxide (ITO), zinc oxide, indium oxide, indium zinc oxide (IZO), or the like.

In the organic optoelectronic device provided by the present disclosure, the second electrode serves as a cathode layer, and the cathode material may be, for example, a material with a small work function, so that electrons are smoothly injected into the organic layer. The cathode material may be, for example, a metal or a multilayer structure material. The metal may be, for example, magnesium, silver, calcium, sodium, potassium, titanium, indium, yttrium, lithium, gadolinium, aluminum, tin and lead, or alloys thereof. The cathode material is preferably selected from magnesium and silver.

In the organic optoelectronic device provided by the present disclosure, the material of the hole injection layer is preferably a material whose highest occupied molecular orbital (HOMO) is between the work function of the anode material and the HOMO of the surrounding organic layer(s), and makes it advantageous for receiving holes from the anode at a low voltage.

In the organic optoelectronic device provided by the present disclosure, the material of the hole transport layer is a material having high mobility for holes, and is suitable for receiving holes from the anode or the hole injection layer and transporting the holes to the light-emitting layer. The material of the hole transport layer includes, but is not limited to, an organic material of arylamine, a conductive polymer, a block copolymer having both a conjugated part and a non-conjugated part, and the like.

In the organic optoelectronic device provided by the present disclosure, the material of the light-emitting layer may generally be selected from materials with good quantum efficiency for fluorescence or phosphorescence, and make it possible to emit light in the visible light region by receiving holes and electrons respectively from the hole transport layer and the electron transport layer and combining the holes with the electrons.

In the organic optoelectronic device provided by the present disclosure, the material of the electron transport layer is a material having high electron mobility, which is suitable for advantageously receiving electrons from the cathode and transporting the electrons to the light-emitting layer.

In the organic optoelectronic device provided by the present disclosure, the material of a cover layer generally has a high refractive index, and thus can help improve the light efficiency of the organic light-emitting device, especially help improve the external light-emitting efficiency.

In the organic optoelectronic device provided by the present disclosure, the organic optoelectronic device is an organic photovoltaic device, an organic light-emitting device, an organic solar cell, an electronic paper, an organic photoreceptor, an organic thin film transistor, etc.

Another aspect of the present disclosure provides a display or lighting device, which includes the organic optoelectronic device of the present disclosure.

The following describes the embodiments of the present disclosure by means of specific examples.

### Synthesis Example

The compound represented by the above formula (I) may be synthesized by a known method, such as cross-coupling reactions using transition metals such as nickel and palladium. Other synthetic methods are C—C, C—N coupling reactions using transition metals such as magnesium or zinc. Among the above reactions, Suzuki or Buchwald reaction is preferable due to mild reaction conditions and superior selectivity of various functional groups. The compounds of the present disclosure are illustrated by the following examples, but are not limited to the compounds and synthetic methods exemplified in these examples. The raw materials and solvents of the present disclosure and some commonly used OLED intermediates and other products were purchased from domestic OLED intermediate manufacturers; various palladium catalysts, ligands and the like were purchased from Sigma-Aldrich Company. ¹H-NMR data was measured using a JEOL (400 MHz) nuclear magnetic resonance instrument; HPLC data was measured using a Shimadzu LC-20AD high performance liquid chromatography instrument.

### Example 1

### Synthesis of Compound 1

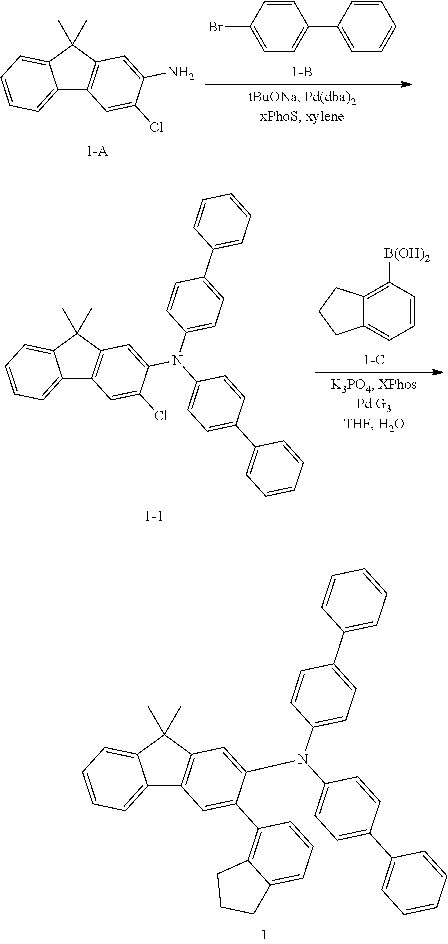 [SMILES: CC1(C)c2ccccc2-c2cc(Cl)c(N)cc21 ; Brc1ccc(-c2ccccc2)cc1 ; CC1(C)c2ccccc2-c2cc(Cl)c(N(c3ccc(-c4ccccc4)cc3)c3ccc(-c4ccccc4)cc3)cc21 ; OB(O)c1cccc2c1CCC2 ; CC1(C)c2ccccc2-c2cc(-c3cccc4c3CCC4)c(N(c3ccc(-c4ccccc4)cc3)c3ccc(-c4ccccc4)cc3)cc21]

### 1) Synthesis of Intermediate 1-1

Under an argon atmosphere, 24.4 g (100 mmol) of compound 1-A, 46.6 g (200 mmol) of compound 1-B, 23.4 g (240 mmol) of sodium tert-butoxide, 575 mg (1 mmol %) of bis(dibenzylideneacetone)palladium, 953 mg (2 mmol %) of 2-dicyclohexylphosphine-2′,4′,6′-triisopropylbiphenyl and 1000 mL of xylene were added to a reaction container, and heated to 140° C. with stirring for 15 hours. The reaction mixture was cooled to room temperature, and 1000 ml of water was added thereto. The mixture was filtered. The filter cake was washed with a large amount of water and dried in vacuum. The crude product was purified by silica gel column chromatography (eluent: ethyl acetate/hexane) to obtain 46.6 g of compound 1-1 with a HPLC purity of 99.5% and a yield of 85%. LC MS: M/Z 547.21 (M+).

¹H NMR (500 MHz, DMSO-d6) δ 7.68-7.62 (m, 4H), 7.61-7.55 (m, 5H), 7.52-7.41 (m, 6H), 7.41-7.31 (m, 4H), 7.27-7.22 (m, 4H), 7.04 (s, 1H), 1.60 (s, 6H).

### 2) Synthesis of Compound 1

Under an argon atmosphere, 54.8 g (101 mmol) of compound 1-1, 16.2 g (100 mmol) of compound 1-C, 787 mg (1 mmol %) of XPhos Pd G3, 50 ml (300 mmol) of 1.5 M potassium phosphate and 1000 ml of tetrahydrofuran (THF) were added to a reaction container and stirred under reflux overnight. After cooling to room temperature, 800 ml of water was added thereto, and a large amount of solid precipitated. The solid was filtered, and the filter cake was washed with water three times and dried in vacuum. The crude product was purified by silica gel column chromatography (eluent: ethyl acetate/hexane) to obtain 54.2 g of compound 1 with a yield of 86% and a HPLC purity of 99.9%. LC-MS: M/Z 629.31 (M+).

¹H NMR (500 MHz, DMSO-d6) δ 7.81 (s, 1H), 7.69-7.63 (m, 4H), 7.60-7.52 (m, 5H), 7.50-7.40 (m, 6H), 7.40-7.30 (m, 4H), 7.30-7.20 (m, 2H), 7.18-7.12 (m, 4H), 7.03 (s, 1H), 2.88 (m, 2H), 2.80 (t, 2H), 2.20 (m, 2H), 1.58 (s, 6H).

### Example 2

### Synthesis of Compound 20

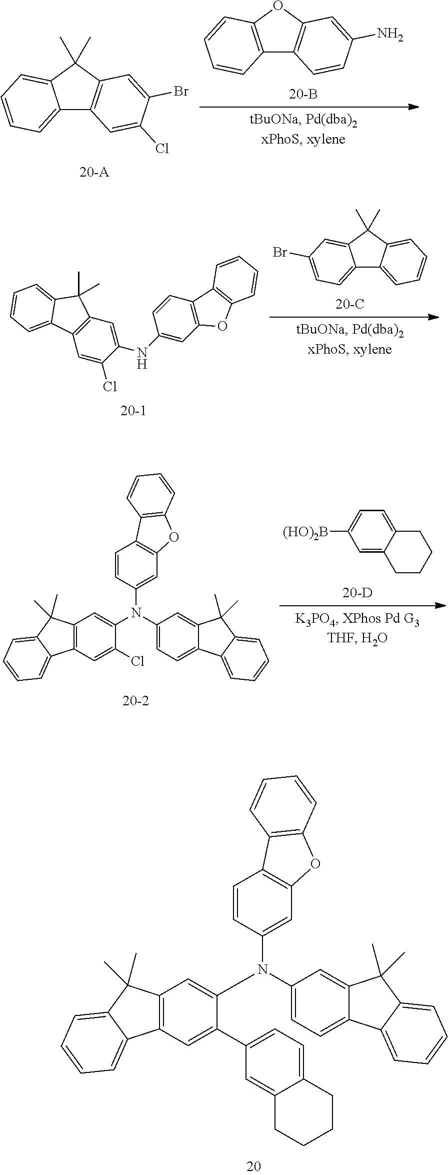

### 1) Synthesis of Intermediate 20-1

Under an argon atmosphere, 30.8 g (100 mmol) of compound 20-A, 18.3 g (100 mmol) of compound 20-B, 23.4 g (240 mmol) of sodium tert-butoxide, 575 mg (1 mmol %) of bis(dibenzylideneacetone)palladium, 953 mg (2 mmol %) of 2-dicyclohexylphosphine-2′,4′,6′-triisopropylbiphenyl and 1000 mL of xylene were added to a reaction container, and heated to 140° C. with stirring for 15 hours. The reaction mixture was cooled to room temperature, and 1000 ml of water was added thereto. The mixture was filtered. The filter cake was washed with a large amount of water and dried in vacuum. The crude product was purified by silica gel column chromatography (eluent: ethyl acetate/hexane) to obtain 32.7 g of compound 20-1 with a HPLC purity of 99.5% and a yield of 80%. LC MS: M/Z 409.12 (M+).

¹H NMR (500 MHz, DMSO-d6) δ 9.55 (s, 1H), 8.06-7.98 (m, 2H), 7.67 (s, 1H), 7.59 (m, 1H), 7.54-7.44 (m, 4H), 7.41-7.29 (m, 3H), 6.90 (s, 1H), 6.86 (m, 1H), 1.58 (s, 6H).

### 2) Synthesis of Intermediate 20-2

Under an argon atmosphere, 40.9 g (100 mmol) of compound 20-1, 27.3 g (100 mmol) of compound 20-C, 23.4 g (240 mmol) of sodium tert-butoxide, 575 mg (1 mmol %) of bis(dibenzylideneacetone)palladium, 953 mg (2 mmol %) of 2-dicyclohexylphosphine-2′,4′,6′-triisopropylbiphenyl and 1000 mL of xylene were added to a reaction container, and heated to 140° C. with stirring for 15 hours. The reaction mixture was cooled to room temperature, and 1000 ml of water was added thereto. The mixture was filtered. The filter cake was washed with a large amount of water and dried in vacuum. The crude product was purified by silica gel column chromatography (eluent: ethyl acetate/hexane) to obtain 48.8 g of compound 20-2 with a HPLC purity of 99.5% and a yield of 86%. LC MS: M/Z 567.26 (M+).

¹H NMR (500 MHz, DMSO-d6) δ 8.10 (m, 1H), 7.95 (d, 1H), 7.75-7.66 (m, 2H), 7.59-7.52 (m, 3H), 7.52-7.31 (m, 9H), 7.28 (m, 1H), 6.93 (d, 1H), 6.88 (m, 1H), 6.84 (s, 1H), 1.58 (s, 12H).

### 3) Synthesis of Compound 20

Under an argon atmosphere, 56.7 g (100 mmol) of compound 20-2, 17.6 g (100 mmol) of compound 20-D, 787 mg (1 mmol %) of XPhos Pd G3, 50 ml (300 mmol) of 1.5 M potassium phosphate and 1000 ml of tetrahydrofuran (THF) were added to a reaction container, and stirred under reflux overnight. After cooling to room temperature, 800 ml of water was added thereto, and a large amount of solid precipitated. The solid was filtered, and the filter cake was washed with water three times and dried in vacuum. The crude product was purified by silica gel column chromatography (eluent: ethyl acetate/hexane) to obtain 55.1 g of compound 20 with a yield of 79% and a HPLC purity of 99.9%. LC MS: M/Z 697.33 (M+).

¹H NMR (500 MHz, DMSO-d6) δ 8.06 (m, 1H), 7.95 (d, 1H), 7.83 (s, 1H), 7.75-7.67 (m, 2H), 7.58-7.51 (m, 2H), 7.51-7.41 (m, 4H), 7.41-7.30 (m, 5H), 7.28 (m, 1H), 7.26-7.17 (m, 2H), 7.08 (d, 1H), 7.04 (s, 1H), 6.93 (d, 1H), 6.88 (m, 1H), 2.79-2.67 (m, 4H), 1.82-1.68 (m, 4H), 1.59 (s, 12H).

### Example 3

### Synthesis of Compound 39

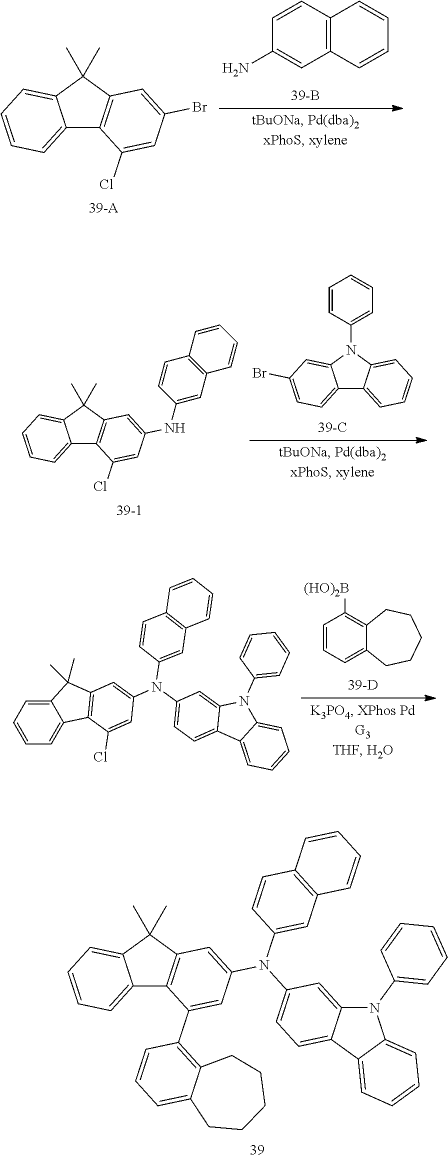

Except that the starting materials were replaced with 39-A, 39-B, 39-C and 39-D, everything else was the same as Example 2. LC MS: M/Z 720.35 (M+). Total yield of synthesis: 52%; HPLC purity: 99.9%.

¹H NMR (500 MHz, DMSO-d6) δ 8.10 (m, 1H), 8.04 (d, 1H), 7.82 (m, 1H), 7.78 (m, 1H), 7.73 (m, 1H), 7.64-7.59 (m, 1H), 7.59-7.22 (m, 19H), 7.17 (m, 1H), 7.15-7.09 (m, 1H), 6.86 (d, 1H), 2.97-2.91 (m, 2H), 2.74 (m, 2H), 1.60 (s, 6H), 1.66-1.52 (m, 6H).

### Example 4

### Synthesis of Compound 58

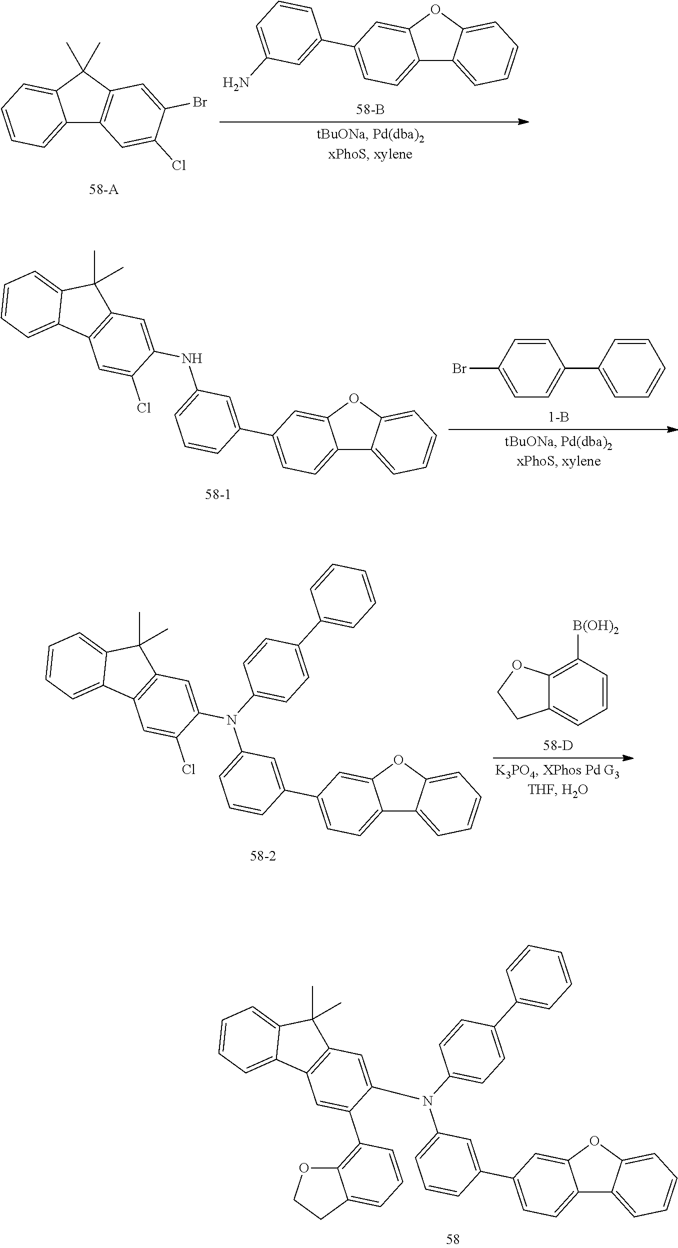

Except that the starting materials were replaced with 58-A, 58-B, 1-B and 58-D, everything else was the same as Example 2. LC MS: M/Z 721.30 (M+). Total yield of synthesis: 49%; HPLC purity: 99.9%.

¹H NMR (500 MHz, DMSO-d6) δ 8.11-8.05 (m, 1H), 7.86 (d, 1H), 7.69-7.63 (m, 2H), 7.60-7.50 (m, 7H), 7.50-7.29 (m, 13H), 7.25-7.18 (m, 1H), 7.18-7.11 (m, 3H), 7.02 (s, 1H), 4.53 (t, 2H), 3.24 (m, 2H), 1.59 (s, 6H).

### Example 5

### Synthesis of Compound 77

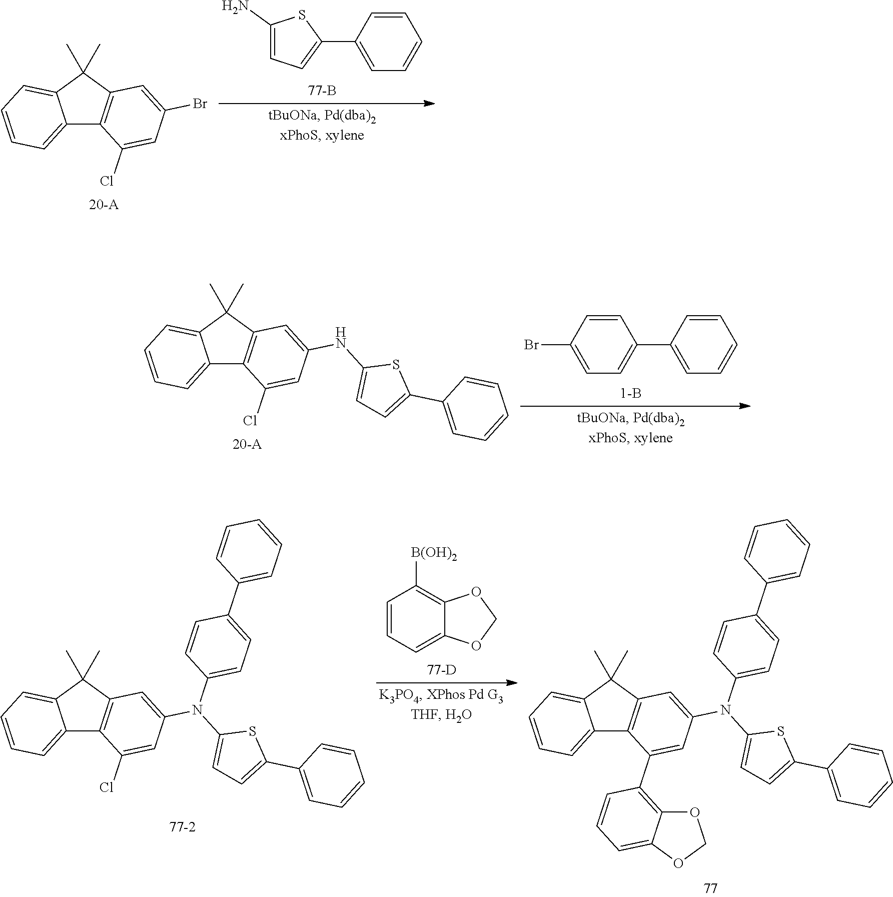

Except that the starting materials were replaced with 77-B, 1-B and 77-D, everything else was the same as Example 2. LC MS: M/Z 639.22 (M+). Total yield of synthesis: 53%; HPLC purity: 99.9%.

¹H NMR (500 MHz, DMSO-d6) δ 7.71 (m, 1H), 7.69-7.63 (m, 4H), 7.58-7.51 (m, 3H), 7.46-7.41 (m, 1H), 7.44-7.30 (m, 8H), 7.30 (m, 1H), 7.20 (t, 1H), 7.17-7.11 (m, 2H), 7.09 (d, 1H), 6.99 (m, 1H), 6.83 (d, 1H), 6.34 (d, 1H), 6.09 (s, 2H), 1.60 (s, 6H).

### Example 6

### Synthesis of Compound 96

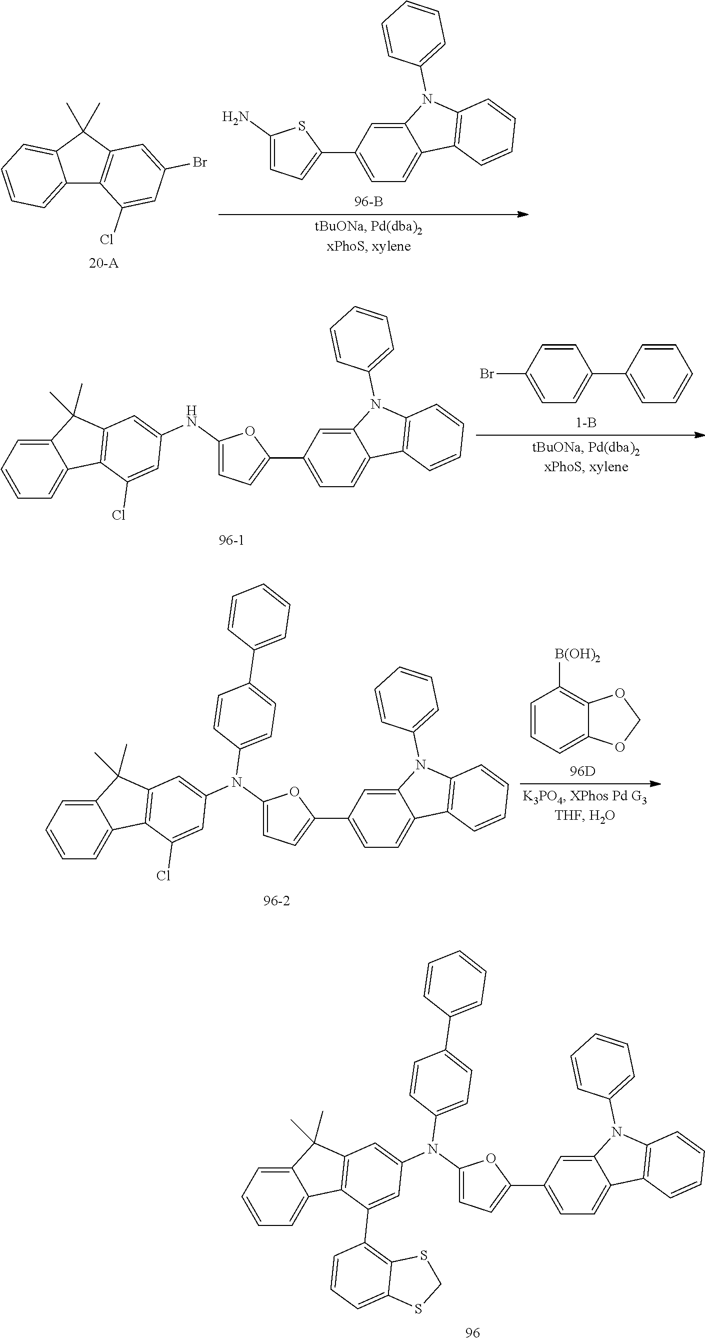

Except that the starting materials were replaced with 96-B, 1-B and 96-D, everything else was the same as Example 2. LC MS: M/Z 820.26 (M+). Total yield of synthesis: 50%; HPLC purity: 99.9%.

¹H NMR (500 MHz, DMSO-d6) δ 8.13 (m,1H), 7.81 (d, 1H), 7.75-7.69 (m, 1H), 7.71-7.64 (m, 3H), 7.64-7.57 (m, 3H), 7.57-7.21 (m, 19H), 7.19 (m, 1H), 7.06 (d, 1H), 6.99 (d, 1H), 6.79 (d, 1H), 4.11 (s, 2H), 1.60 (s, 6H).

### Example 7

### Synthesis of Compound 115

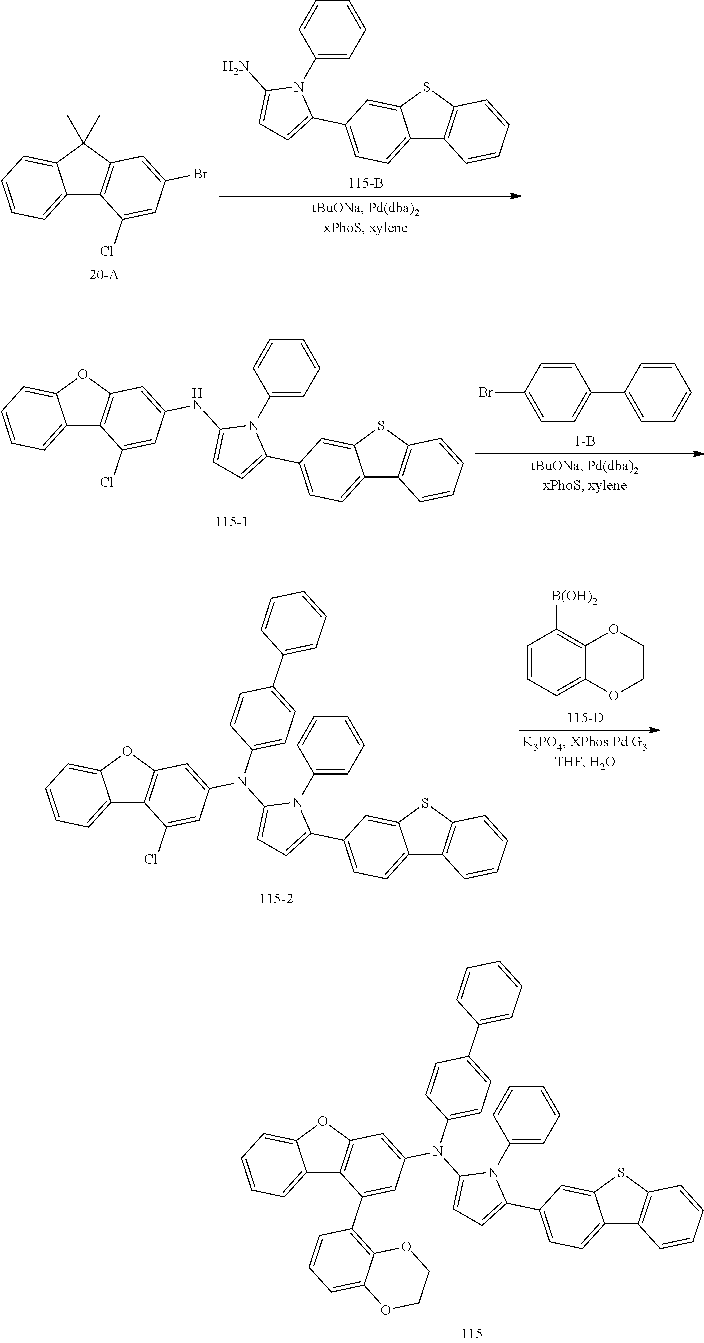

Except that the starting materials were replaced with 115-B, 1-B and 115-D, everything else was the same as Example 2. LC MS: M/Z 792.24 (M+). Total yield of synthesis: 48%; HPLC purity: 99.9%.

¹H NMR (500 MHz, DMSO-d6) δ 8.30 (m, 1H), 8.07 (d, 1H), 8.04-7.97 (m, 1H), 7.95 (m, 2H), 7.81 (m, 1H), 7.69-7.63 (m, 2H), 7.62-7.32 (m, 17H), 7.22-7.16 (m, 2H), 7.13 (t, 1H), 7.00 (m, 1H), 6.94 (d, 1H), 6.58 (d, 1H), 5.88 (d, 1H), 4.34-4.24 (m, 4H).

### Example 8

### Synthesis of Compound 134

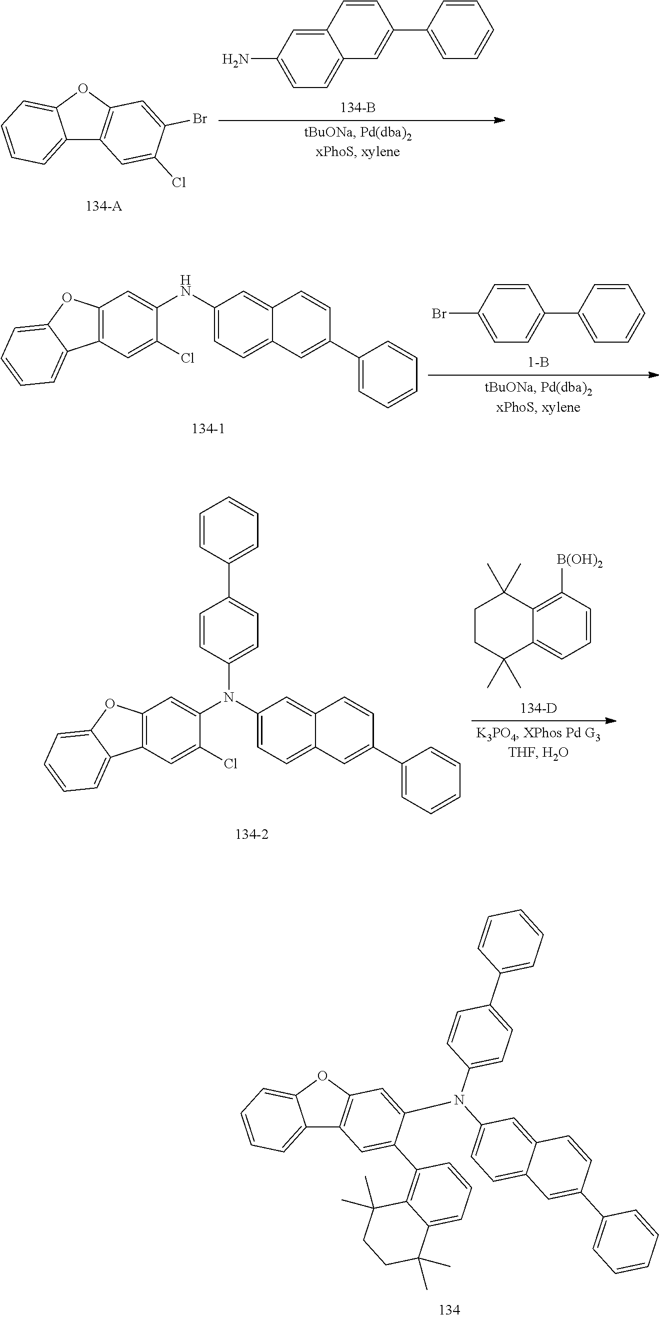

Except that the starting materials were replaced with 134-A, 134-B, 1-B and 134-D, everything else was the same as Example 2. LC MS: M/Z 723.35 (M+). Total yield of synthesis: 45%; HPLC purity: 99.9%.

¹H NMR (500 MHz, DMSO-d6) δ 8.13 (m, 1H), 8.01 (s, 1H), 7.82 (m, 1H), 7.69-7.63 (m, 2H), 7.63-7.58 (m, 2H), 7.58-7.51 (m, 5H), 7.51-7.26 (m, 9H), 7.18-7.08 (m, 4H), 1.85-1.78 (m, 2H), 1.78-1.71 (m, 2H), 1.31 (s, 4H), 0.94 (s, 12H).

### Example 9

### Synthesis of Compound 153

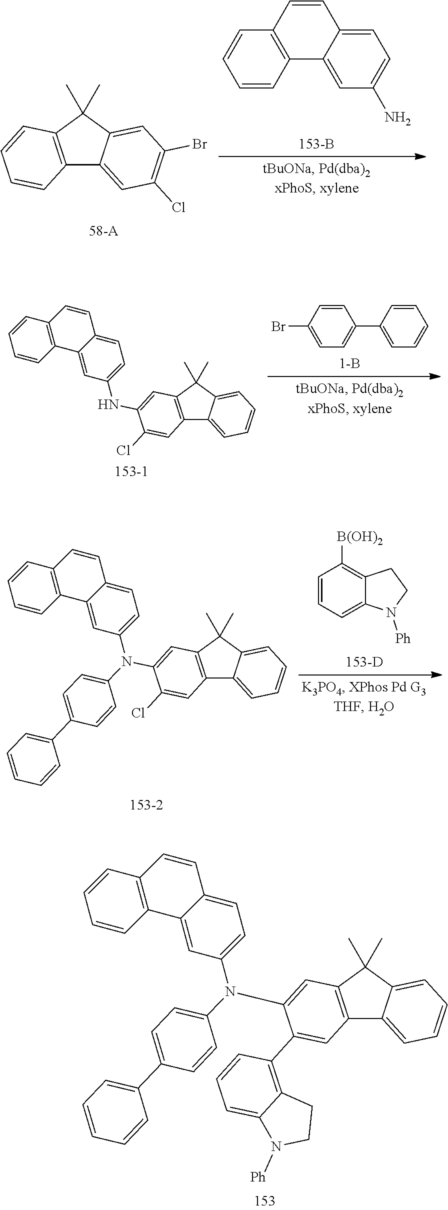

Except that the starting materials were replaced with 58-A, 153-B and 153-D, everything else was the same as Example 2. LC MS: M/Z 730.33 (M+). Total yield of synthesis: 50%; HPLC purity: 99.9%.

¹H NMR (500 MHz, DMSO-d6) δ 8.06-8.01 (m, 1H), 7.98 (m, 1H), 7.92-7.84 (m, 3H), 7.78 (m, 1H), 7.69-7.63 (m, 2H), 7.61 (m, 1H), 7.58-7.49 (m, 4H), 7.49-7.27 (m, 12H), 7.15 (m, 4H), 7.13-7.05 (m, 2H), 7.01 (s, 1H), 4.12 (t, 2H), 3.17 (m, 1H), 3.09 (m, 1H), 1.59 (s, 6H).

### Example 10

### Synthesis of Compound 172

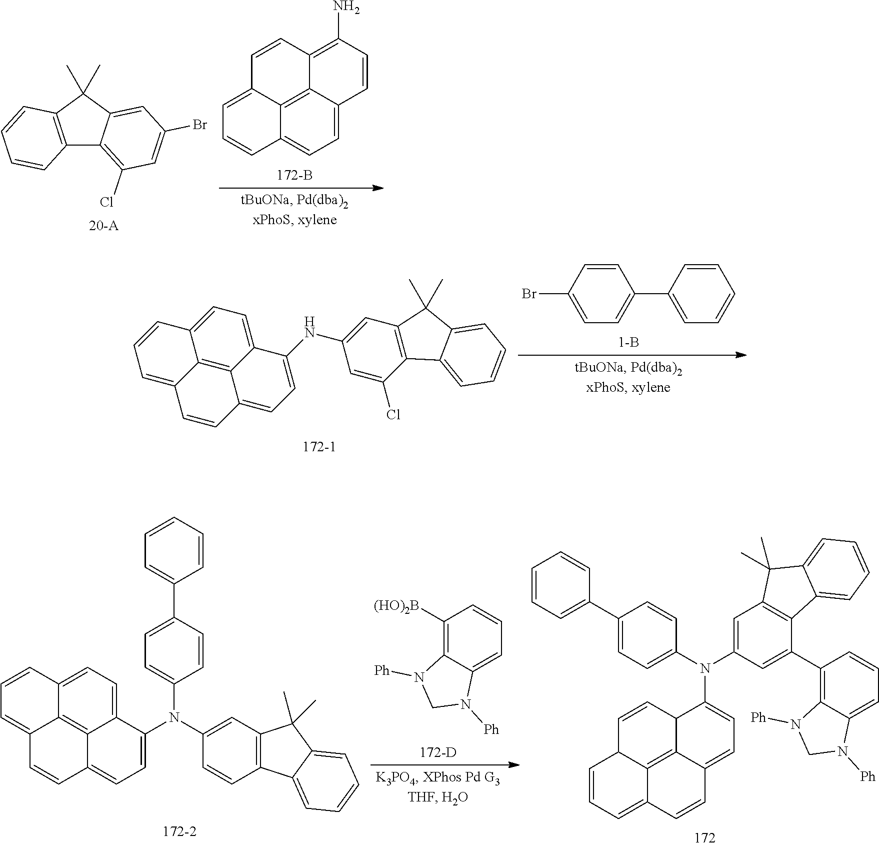

Except that the starting materials were replaced with 172-B and 172-D, everything else was the same as Example 2. LC MS: M/Z 831.36 (M+). Total yield of synthesis: 47%; HPLC purity: 99.9%.

¹H NMR (500 MHz, DMSO-d6) δ 8.32-8.25 (m, 1H), 8.06 (m, 1H), 8.06-7.99 (m, 2H), 7.91 (m, 1H), 7.76 (m, 1H), 7.73-7.63 (m, 5H), 7.57-7.52 (m, 2H), 7.50 (m, 1H), 7.46-7.33 (m, 10H), 7.36-7.27 (m, 3H), 7.27-7.07 (m, 9H), 6.86 (d, 1H), 4.69 (s, 2H), 1.60 (s, 6H).

### Example 11

### Synthesis of Compound 191

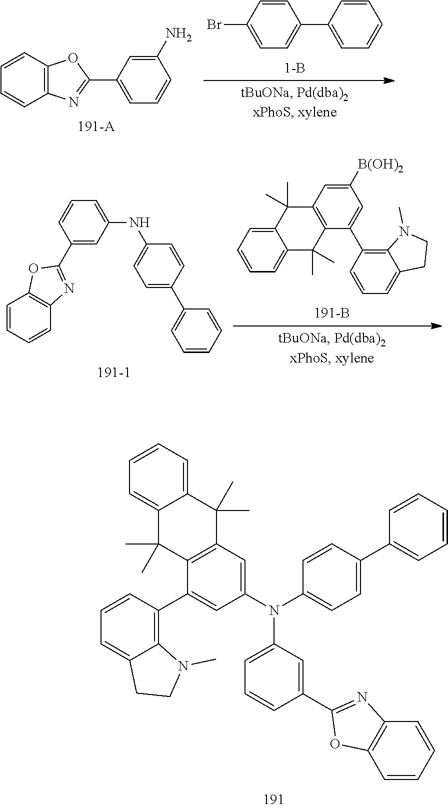 [SMILES: Nc1cccc(-c2nc3ccccc3o2)c1 ; Brc1ccc(-c2ccccc2)cc1 ; c1ccc(-c2ccc(Nc3cccc(-c4nc5ccccc5o4)c3)cc2)cc1 ; CN1CCc2cccc(-c3cc(B(O)O)cc4c3C(C)(C)c3ccccc3C4(C)C)c21 ; CN1CCc2cccc(-c3cc(N(c4ccc(-c5ccccc5)cc4)c4cccc(-c5nc6ccccc6o5)c4)cc4c3C(C)(C)c3ccccc3C4(C)C)c21]

Except that the starting materials were replaced with 191-A and 191-B, everything else was the same as Example 1. LC MS: M/Z 727.36 (M+). Total yield of synthesis: 45%; HPLC purity: 99.9%.

¹H NMR (500 MHz, DMSO-d6) δ 7.73 (m, 1H), 7.71-7.63 (m, 3H), 7.61 (t, 1H), 7.57-7.52 (m, 2H), 7.52-7.38 (m, 7H), 7.41-7.31 (m, 2H), 7.23 (t, 1H), 7.20-7.13 (m, 5H), 7.12-6.99 (m, 3H), 6.85 (d, 1H), 3.64 (t, 2H), 2.99 (s, 3H), 2.98 (m, 2H), 1.61 (d, 12H).

### Example 12

### Synthesis of Compound 210

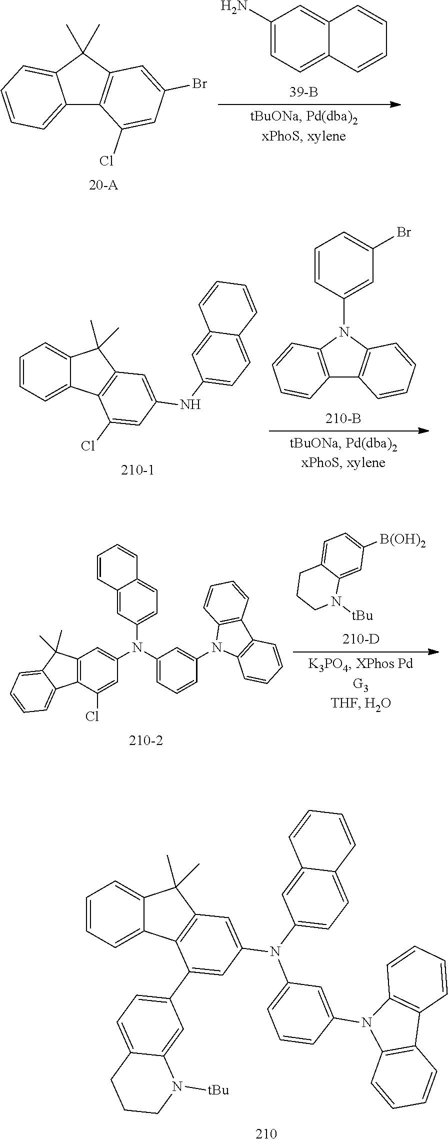

Except that the starting materials were replaced with 39-B, 210-B and 210-D, everything else was the same as Example 2. LC MS: M/Z 763.39 (M+). Total yield of synthesis: 45%; HPLC purity: 99.9%.

¹H NMR (500 MHz, DMSO-d6) δ 8.12 (m, 2H), 7.85-7.75 (m, 2H), 7.71 (m, 1H), 7.62 (m, 2H), 7.60-7.54 (m, 1H), 7.51-7.24 (m, 17H), 7.21-7.12 (m, 4H), 7.08 (m, 1H), 6.88 (d, 1H), 3.41 (t, 2H), 2.79 (m, 2H), 1.90 (m, 2H), 1.60 (s, 6H), 1.33 (s, 6H).

### Example 13

### Synthesis of Compound 229

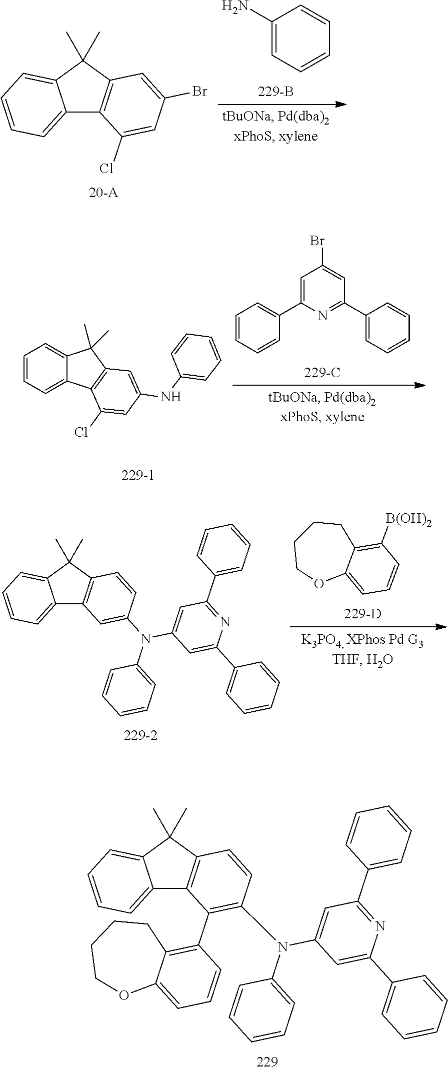

Except that the starting materials were replaced with 229-B, 229-C and 229-D, everything else was the same as Example 2. LC MS: M/Z 660.31 (M+). Total yield of synthesis: 50%; HPLC purity: 99.9%.

¹H NMR (500 MHz, DMSO-d6) δ 8.17-8.11 (m, 4H), 7.72 (m, 1H), 7.55-7.49 (m, 4H), 7.52-7.24 (m, 12H), 7.22 (d, 1H), 7.18-7.12 (m, 2H), 7.15-7.07 (m, 1H), 6.89 (m, 1H), 3.96 (t, 2H), 2.99 (t, 1H), 2.89 (t, 1H), 1.78 (m, 2H), 1.69 (m, 2H), 1.59 (s, 6H).

### Example 14

### Synthesis of Compound 248

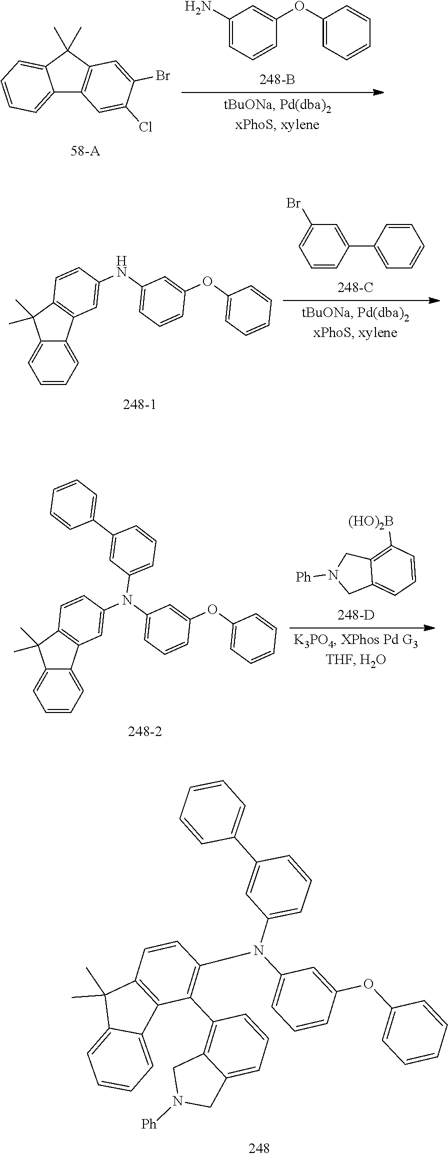

Except that the starting materials were replaced with 58-A, 248-B, 248-C and 248-D, everything else was the same as Example 2. LC MS: M/Z 722.33 (M+). Total yield of synthesis: 48%; HPLC purity: 99.9%.

### Example 15

### Synthesis of Compound 267

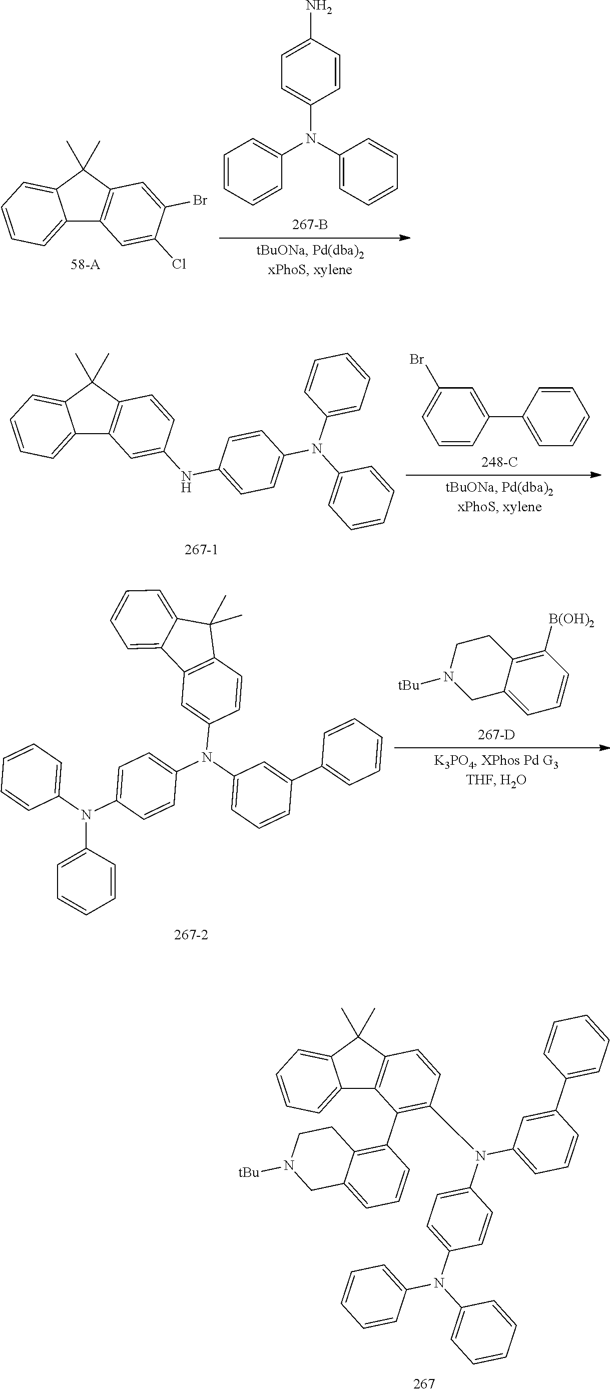

Except that the starting materials were replaced with 58-A, 267-B, 248-C and 267-D, everything else was the same as Example 2. LC MS: M/Z 791.42 (M+). Total yield of synthesis: 49%; HPLC purity: 99.9%.

¹H NMR (500 MHz, DMSO-d6) δ 7.71 (m, 1H), 7.60 (m, 1H), 7.56-7.48 (m, 3H), 7.46 (t, 1H), 7.43-7.19 (m, 16H), 7.19-7.11 (m, 2H), 6.98 (m, 4H), 6.91 (m, 2H), 6.73 (t, 1H), 6.55 (m, 1H), 4.60-4.56 (m, 2H), 4.50 (s, 2H), 1.59 (s, 6H).

### Example 16

### Synthesis of Compound 286

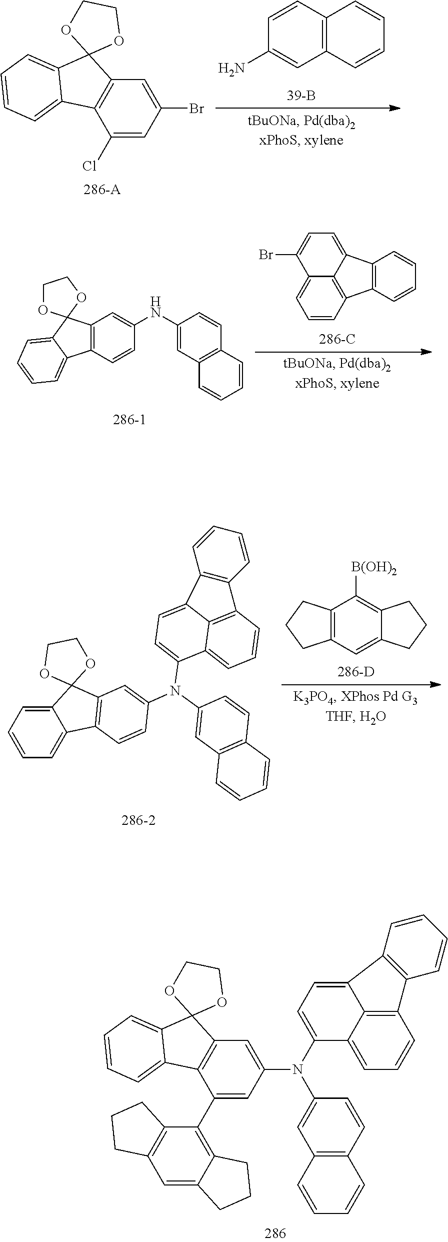

Except that the starting materials were replaced with 286-A, 39-B, 286-C and 286-D, everything else was the same as Example 2. LC MS: M/Z 721.30 (M+). Total yield of synthesis: 47%; HPLC purity: 99.9%.

¹H NMR (500 MHz, DMSO-d6) δ 8.02 (m, 2H), 7.90 (m, 2H), 7.86-7.75 (m, 3H), 7.72 (d, 1H), 7.61-7.54 (m, 3H), 7.54-7.41 (m, 4H), 7.39-7.30 (m, 3H), 7.33-7.25 (m, 2H), 7.11-7.04 (m, 2H), 6.96 (d, 1H), 4.16-4.03 (m, 4H), 2.87 (m, 4H), 2.77 (t, 4H), 2.19 (m, 4H).

### Example 17

### Synthesis of Compound 305

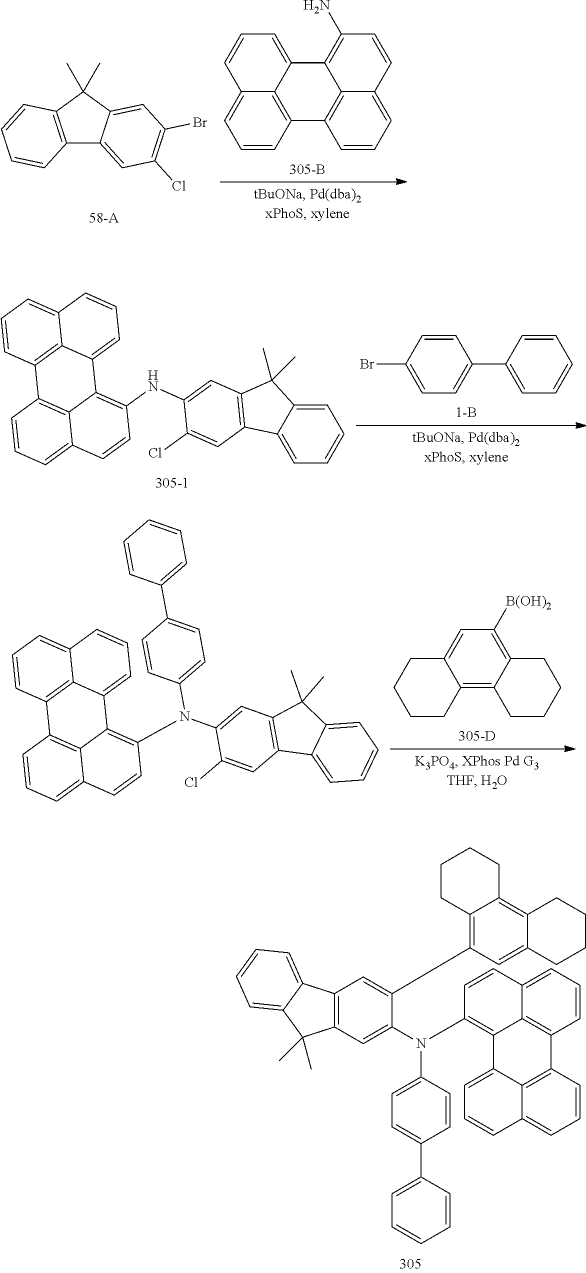

Except that the starting materials were replaced with 58-A, 305-B, 1-B and 305-D, everything else was the same as Example 2. LC MS: M/Z 795.39 (M+). Total yield of synthesis: 54%; HPLC purity: 99.9%.

¹H NMR (500 MHz, DMSO-d6) δ 8.33 (m, 1H), 8.19 (m, 2H), 7.80-7.63 (m, 7H), 7.58-7.44 (m, 7H), 7.44-7.28 (m, 5H), 7.25 (d, 1H), 7.21-7.15 (m, 2H), 7.11 (t, 1H), 7.07 (s, 1H), 2.98-2.90 (m, 2H), 2.83-2.73 (m, 4H), 2.71 (m, 2H), 1.82-1.68 (m, 8H), 1.59 (s, 6H).

### Example 18

### Synthesis of Compound 324

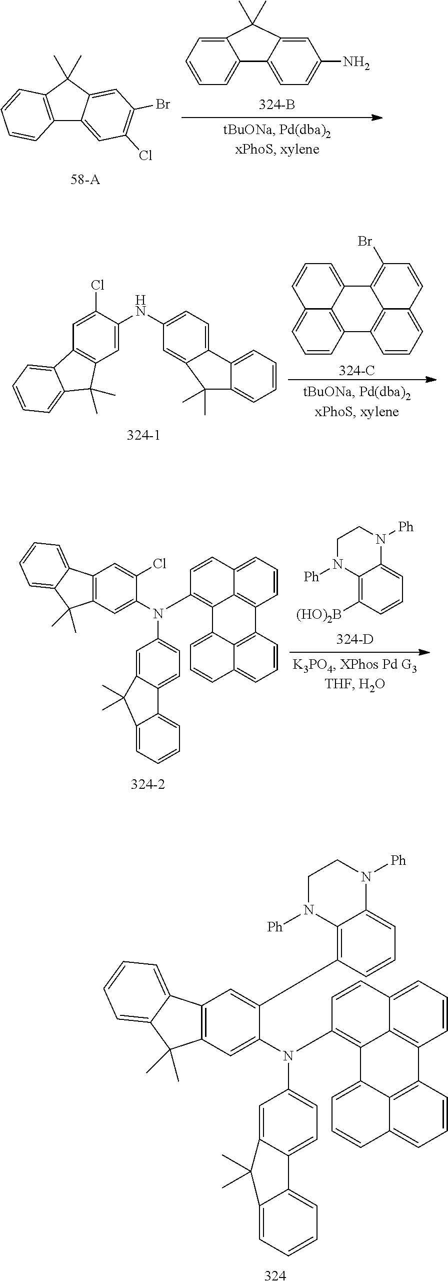

Except that the starting materials were replaced with 58-A, 324-B, 324-C and 324-D, everything else was the same as Example 1. LC MS: M/Z 935.42 (M+). Total yield of synthesis: 51%; HPLC purity: 99.9%.

¹H NMR (500 MHz, DMSO-d6) δ 8.33 (m, 1H), 8.21 (m, 1H), 8.17 (m, 1H), 7.75-7.66 (m, 8H), 7.58-7.43 (m, 7H), 7.40-7.22 (m, 11H), 7.21-7.13 (m, 5H), 7.09 (m, 3H), 7.00 (s, 1H), 6.96 (d, 1H), 4.07 (s, 3H), 4.07 (d, 1H), 1.59 (s, 10H).

### Example 19

### Synthesis of Compound 343

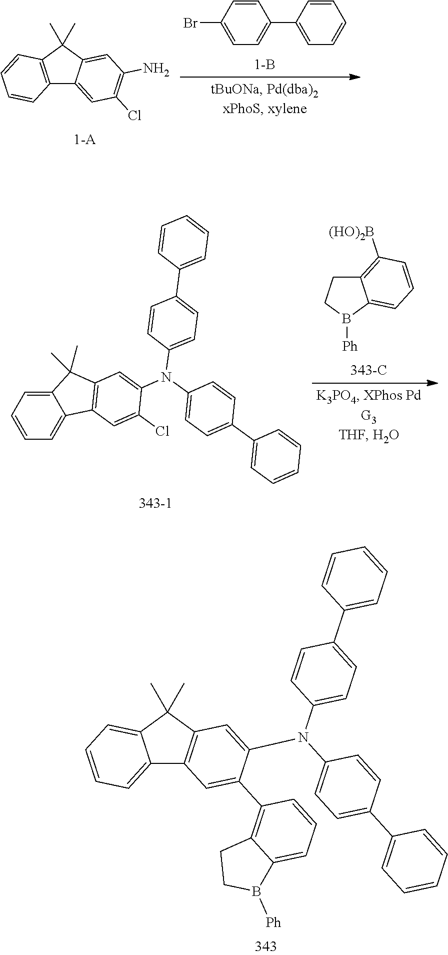 [SMILES: CC1(C)c2ccccc2-c2cc(Cl)c(N)cc21 ; Brc1ccc(-c2ccccc2)cc1 ; CC1(C)c2ccccc2-c2cc(Cl)c(N(c3ccc(-c4ccccc4)cc3)c3ccc(-c4ccccc4)cc3)cc21 ; OB(O)c1cccc2c1CCB2c1ccccc1 ; CC1(C)c2ccccc2-c2cc(-c3cccc4c3CCB4c3ccccc3)c(N(c3ccc(-c4ccccc4)cc3)c3ccc(-c4ccccc4)cc3)cc21]

Except that the starting material was replaced with 343-C, everything else was the same as Example 1. LC MS: M/Z 703.34 (M+). Total yield of synthesis: 50%; HPLC purity: 99.9%.

¹H NMR (500 MHz, DMSO-d6) δ 7.74-7.63 (m, 7H), 7.58-7.49 (m, 6H), 7.49-7.44 (m, 2H), 7.44-7.28 (m, 11H), 7.18 (t, 1H), 7.14-7.08 (m, 4H), 7.06 (s, 1H), 2.81 (m, 1H), 2.65 (t, 2H), 1.94 (t, 1H), 1.59 (s, 6H).

### Example 20

### Synthesis of Compound 362

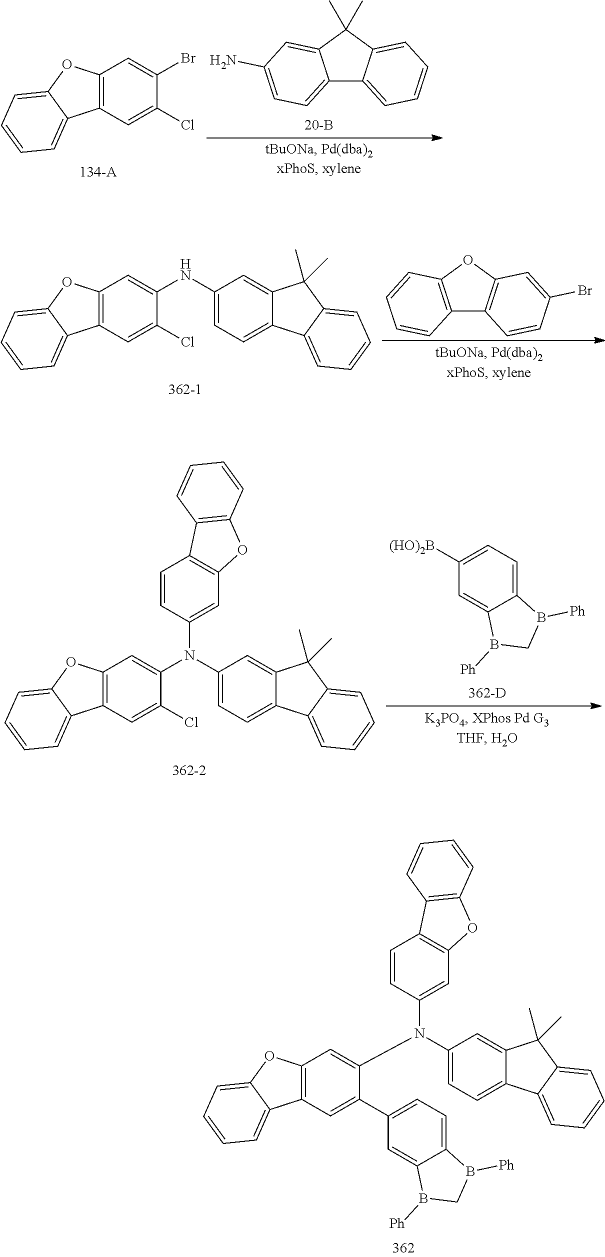

Except that the starting materials were replaced with 134-A, 20-B, and 362D, everything else was the same as Example 13. LC MS: M/Z 729.27 (M+). Total yield of synthesis: 49%; HPLC purity: 99.9%.

¹H NMR (500 MHz, DMSO-d6) δ8.09-8.04 (m, 1H), 8.03 (d, 2H), 7.91 (d, 1H), 7.79 (d, 1H), 7.76-7.66 (m, 7H), 7.58-7.52 (m, 2H), 7.51-7.41 (m, 6H), 7.41-7.31 (m, 6H), 7.34-7.29 (m, 3H), 7.25 (m, 1H), 7.08 (d, 1H), 6.97 (d, 1H), 6.88 (m, 1H), 3.28 (s, 2H), 1.59 (s, 6H).

### Example 21

### Synthesis of Compound 381

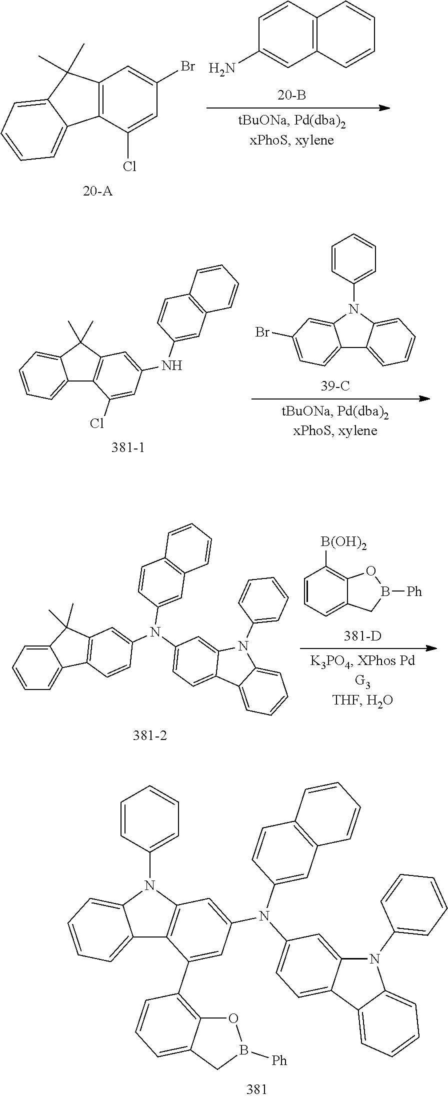

Except that the starting materials were replaced with 39-C and 381-D, everything else was the same as Example 2. LC MS: M/Z 768.77 (M+). Total yield of synthesis: 50%; HPLC purity: 99.9%.

¹H NMR (500 MHz, DMSO-d6) δ 8.10 (m, 1H), 8.04 (d, 1H), 7.85-7.80 (m, 1H), 7.80-7.72 (m, 3H), 7.69 (m, 1H), 7.62 (m, 1H), 7.59-7.45 (m, 5H), 7.47-7.42 (m, 2H), 7.45-7.33 (m, 5H), 7.35 (d, 3H), 7.36-7.30 (m, 2H), 7.33-7.25 (m, 4H), 7.27-7.20 (m, 2H), 7.18 (m, 1H), 7.09 (d, 1H), 3.44 (d, 2H), 1.60 (s, 6H).

### Example 22

### Synthesis of Compound 400

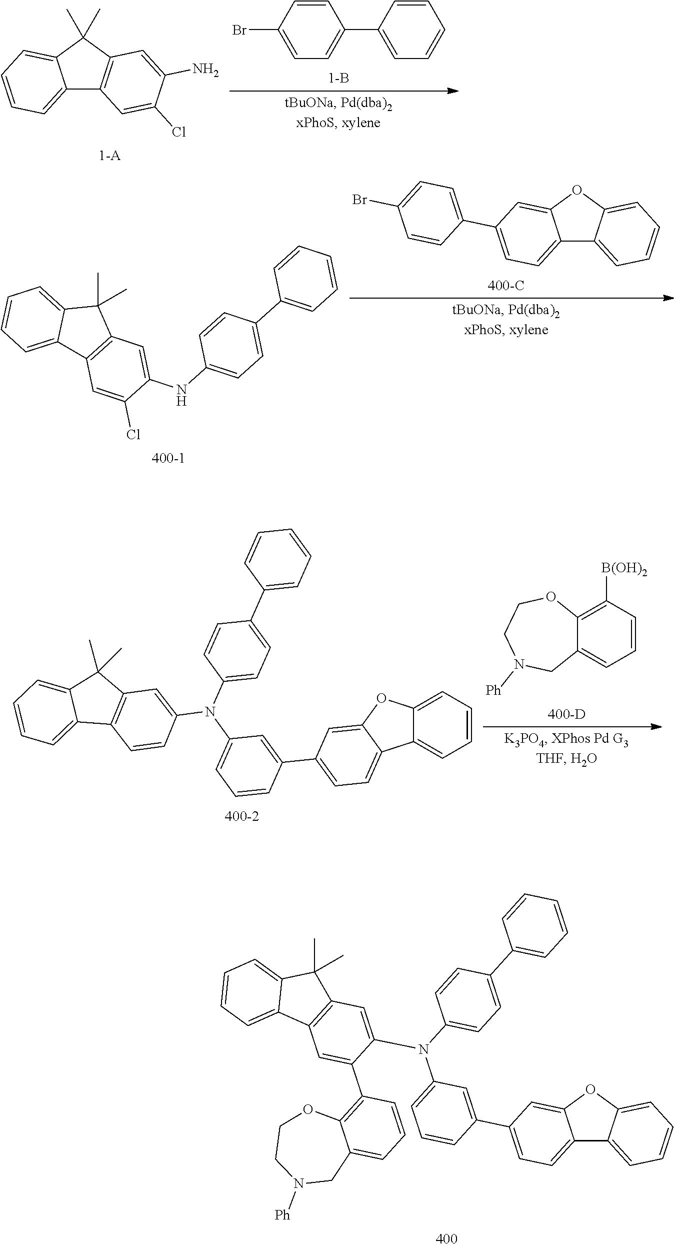

Except that the starting materials were replaced with 1-A, 1-B, 400-C and 400-D, everything else was the same as Example 2. LC MS: M/Z 826.36 (M+). Total yield of synthesis: 51%; HPLC purity: 99.9%.

¹H NMR (500 MHz, DMSO-d6) δ 8.03 (m, 1H), 7.84 (d, 1H), 7.69-7.63 (m, 2H), 7.60-7.50 (m, 7H), 7.50-7.42 (m, 5H), 7.44-7.39 (m, 2H), 7.42-7.32 (m, 5H), 7.35-7.28 (m, 1H), 7.31-7.25 (m, 1H), 7.28-7.21 (m, 2H), 7.14 (m, 3H), 7.02 (s, 1H), 6.98-6.92 (m, 2H), 6.91 (m, 1H), 4.57 (d, 2H), 4.18 (t, 2H), 3.63 (t, 2H), 1.59 (s, 6H).

### Example 23

### Synthesis of Compound 419

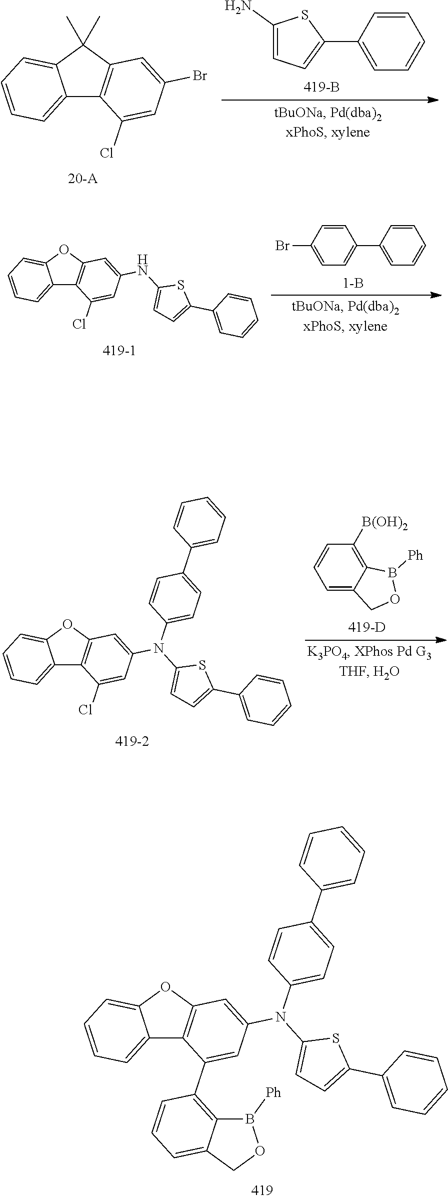

Except that the starting materials were replaced with 419-B, 1-B and 419-D, everything else was the same as Example 2. LC MS: M/Z 685.22 (M+). Total yield of synthesis: 53%; HPLC purity: 99.9%.

¹H NMR (500 MHz, DMSO-d6) δ 8.04-7.96 (m, 2H), 7.74-7.68 (m, 2H), 7.66 (m, 4H), 7.61-7.53 (m, 5H), 7.48-7.27 (m, 12H), 7.17-7.11 (m, 2H), 6.98 (d, 1H), 6.83 (d, 1H), 6.34 (d, 1H), 5.14 (d, 2H).

### Example 24

### Synthesis of Compound 438

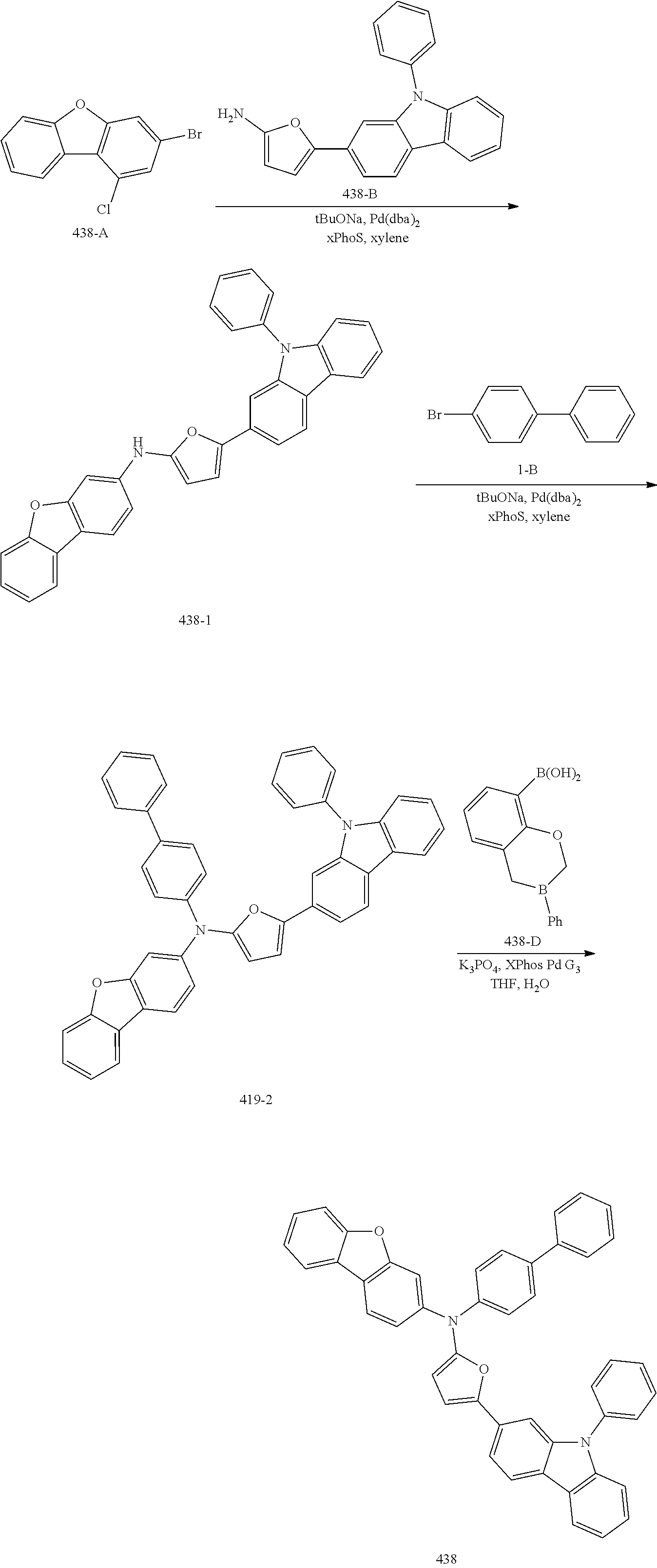

Except that the starting materials were replaced with 438-A, 438-B, 1-B and 438-D, everything else was the same as Example 2. LC MS: M/Z 642.23 (M+). Total yield of synthesis: 47%; HPLC purity: 99.9%.

¹H NMR (500 MHz, DMSO-d6) δ 8.13 (m, 1H), 8.06 (m, 1H), 7.91 (d, 1H), 7.84 (d, 1H), 7.71 (d, 1H), 7.69-7.39 (m, 17H), 7.39-7.32 (m, 1H), 7.32-7.24 (m, 4H), 7.19 (m, 1H), 7.06 (d, 1H), 6.73 (d, 1H).

### Example 25

### Synthesis of Compound 449

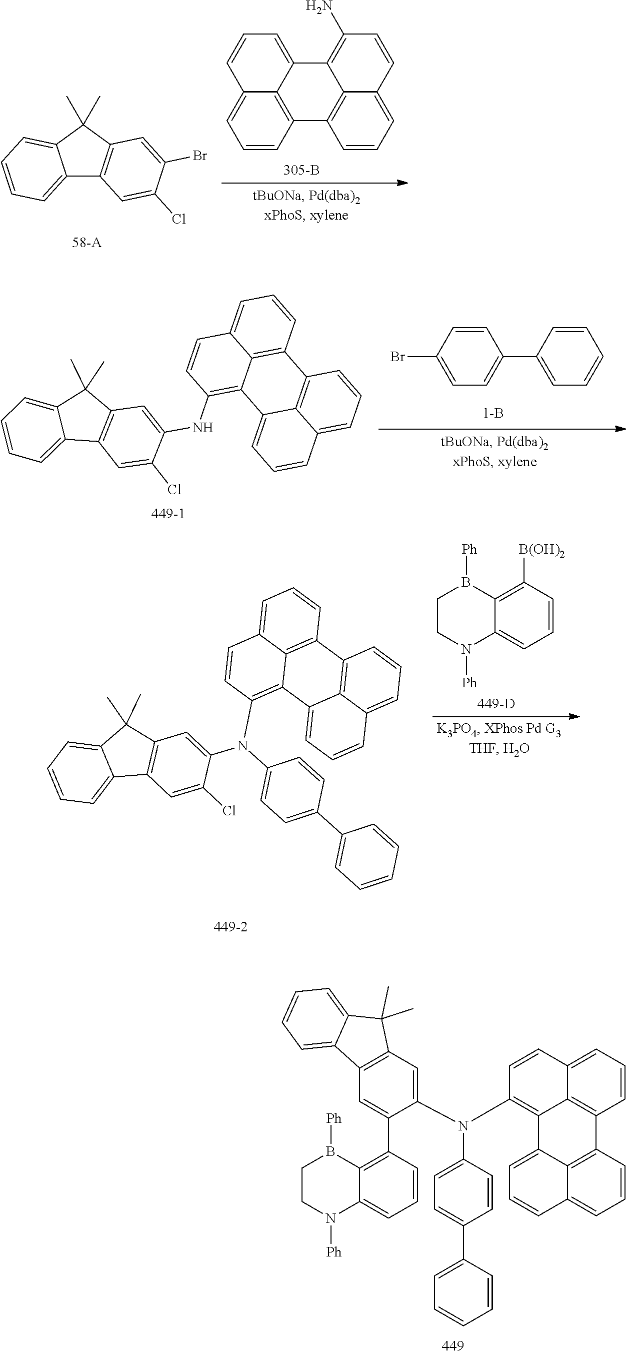

Except that the starting materials were replaced with 58-A, 305-B, 1-B and 449-D, everything else was the same as Example 2. LC MS: M/Z 892.40 (M+). Total yield of synthesis: 49%; HPLC purity: 99.9%.

¹H NMR (500 MHz, DMSO-d6) δ 8.33 (m, 1H), 8.21 (m, 1H), 8.17 (m, 1H), 7.78 (s, 1H), 7.75-7.63 (m, 8H), 7.58-7.44 (m, 7H), 7.44-7.25 (m, 13H), 7.25-7.16 (m, 4H), 7.15 (m, 1H), 7.10 (m, 1H), 7.03 (s, 1H), 3.97 (t, 2H), 2.66 (t, 2H), 1.59 (s, 6H).

### Example 26

### Synthesis of Compound 2

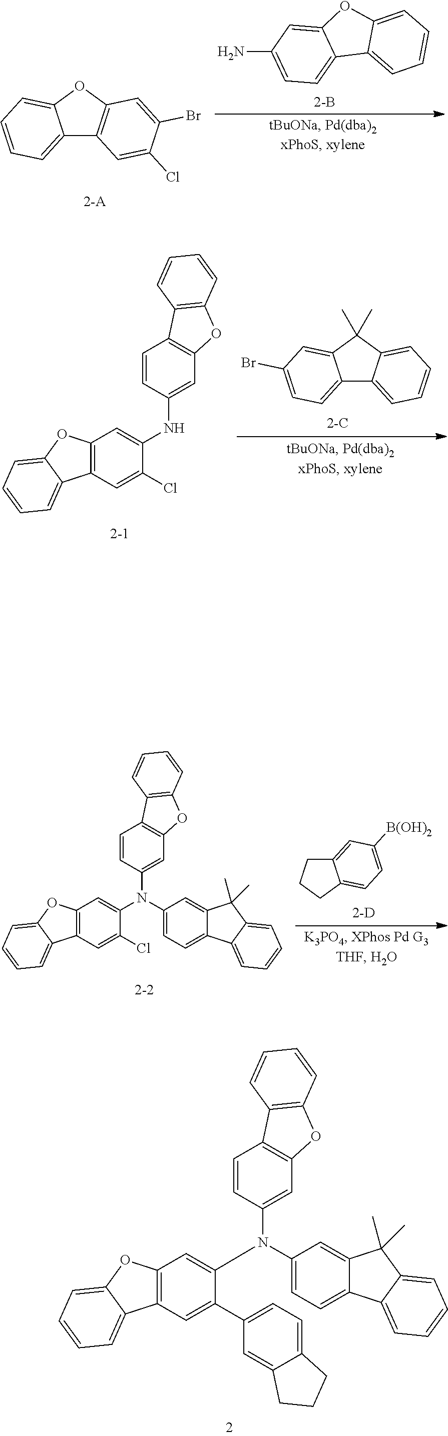

Except that the starting materials were replaced with 2-A, 2-B, 2-C and 2-D, everything else was the same as Example 2. LC MS: M/Z 657.27 (M+). Total yield of synthesis: 47%; HPLC purity: 99.9%.

¹H NMR (500 MHz, DMSO-d6) δ 8.16 (m, 1H), 8.06 (m, 1H), 8.00 (s, 1H), 7.92 (d, 1H), 7.76-7.68 (m, 2H), 7.55 (m, 2H), 7.52-7.40 (m, 6H), 7.40-7.27 (m, 5H), 7.24 (m, 1H), 7.10 (d, 1H), 6.93 (d, 1H), 6.88 (m, 1H), 2.92-2.84 (m, 4H), 2.14 (m, 2H), 1.58 (s, 6H).

### Example 27

### Synthesis of Compound 8

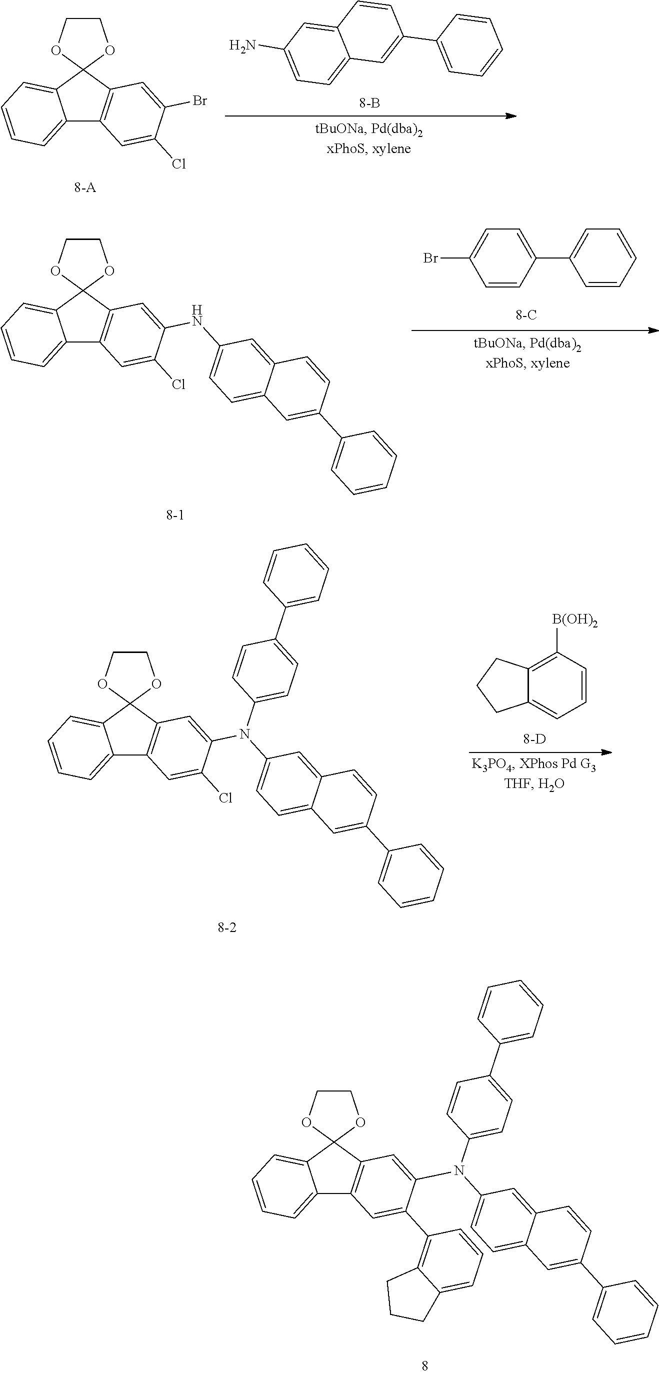

Except that the starting materials were replaced with 8-A, 8-B, 8-C and 8-D, everything else was the same as Example 2. LC MS: M/Z 709.30 (M+). Total yield of synthesis: 46%; HPLC purity: 99.9%.

¹H NMR (500 MHz, DMSO-d6) δ 7.82 (m, 1H), 7.74-7.68 (m, 2H), 7.68-7.63 (m, 2H), 7.63-7.58 (m, 2H), 7.58-7.51 (m, 4H), 7.45-7.35 (m, 7H), 7.35 (m, 4H), 7.35-7.24 (m, 2H), 7.28-7.20 (m, 2H), 7.19-7.12 (m, 3H), 4.11 (s, 4H), 2.92-2.85 (m, 2H), 2.78 (t, 2H), 2.18 (m, 2H).

### Example 28

### Synthesis of Compound 11

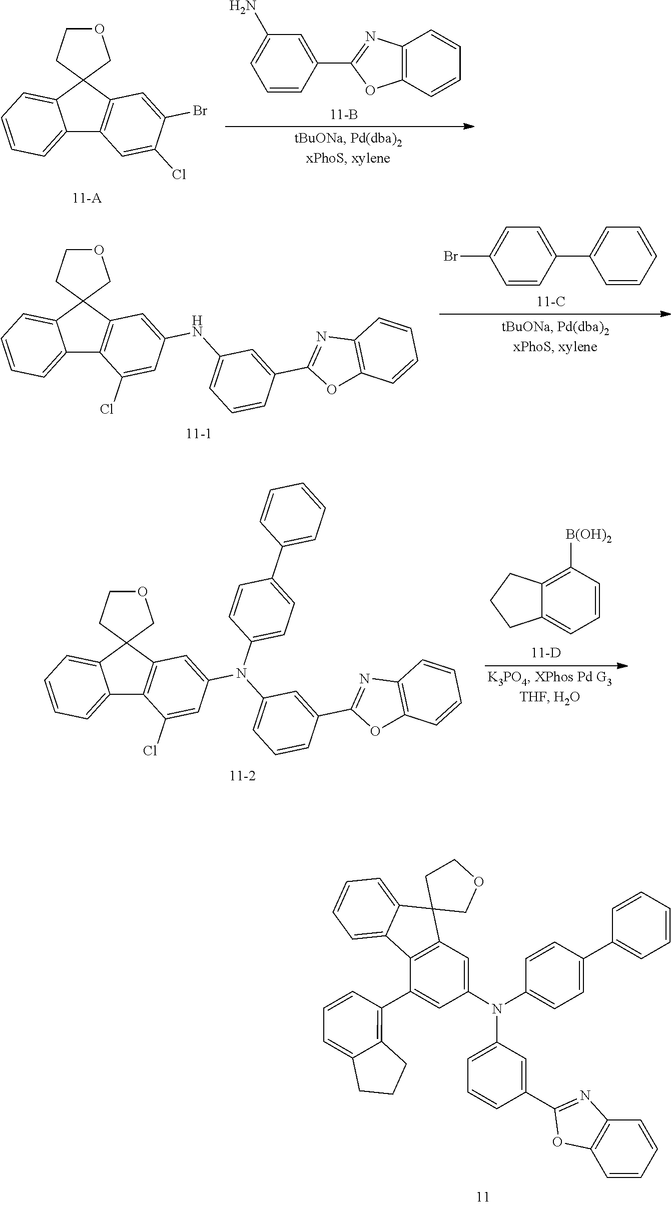

Except that the starting materials were replaced with 11-A, 11-B, 11-C and 11-D, everything else was the same as Example 2. LC MS: M/Z 698.29 (M+). Total yield of synthesis: 46%; HPLC purity: 99.9%.

¹H NMR (500 MHz, DMSO-d6) δ 7.75 (m, 1H), 7.73-7.63 (m, 3H), 7.62 (t, 1H), 7.58-7.53 (m, 2H), 7.53-7.24 (m, 15H), 7.21-7.13 (m, 3H), 6.87 (d, 1H), 4.22-4.16 (m, 1H), 4.16-4.09 (m, 1H), 3.91 (t, 2H), 2.95-2.80 (m, 2H), 2.78-2.69 (m, 2H), 2.72-2.55 (m, 2H), 2.25-2.08 (m, 2H).

### Example 29

### Synthesis of Compound 128

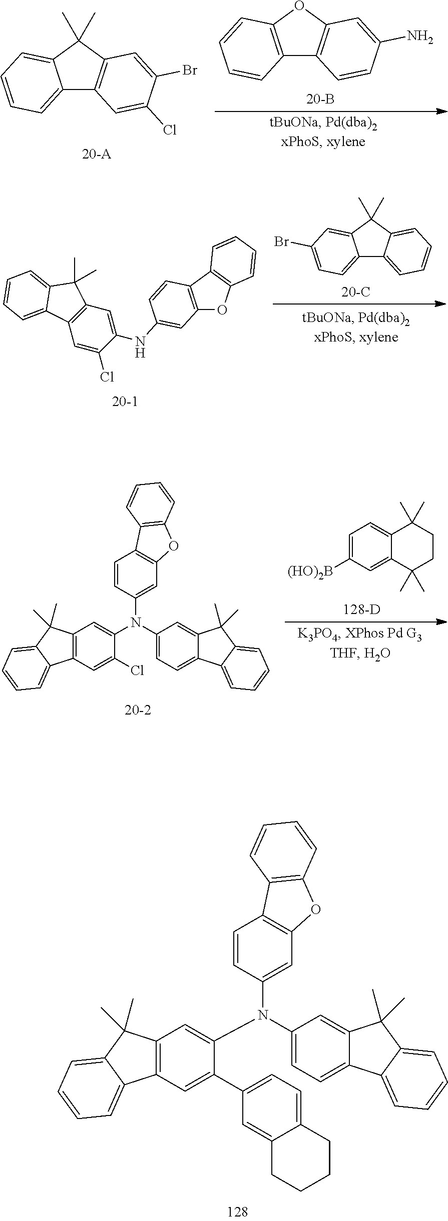

Except that the starting material was replaced with 128-D, everything else was the same as Example 2. LC MS: M/Z 697.33 (M+). Total yield of synthesis: 46%; HPLC purity: 99.9%.

¹H NMR (500 MHz, DMSO-d6) δ 8.06 (m, 1H), 7.95 (d, 1H), 7.83 (s, 1H), 7.75-7.67 (m, 2H), 7.58-7.51 (m, 2H), 7.51-7.41 (m, 4H), 7.41-7.30 (m, 5H), 7.28 (m, 1H), 7.26-7.17 (m, 2H), 7.08 (d, 1H), 7.04 (s, 1H), 6.93 (d, 1H), 6.88 (m, 1H), 2.79-2.67 (m, 4H), 1.82-1.68 (m, 4H), 1.59 (s, 12H).

### Example 30

### Synthesis of Compound 130

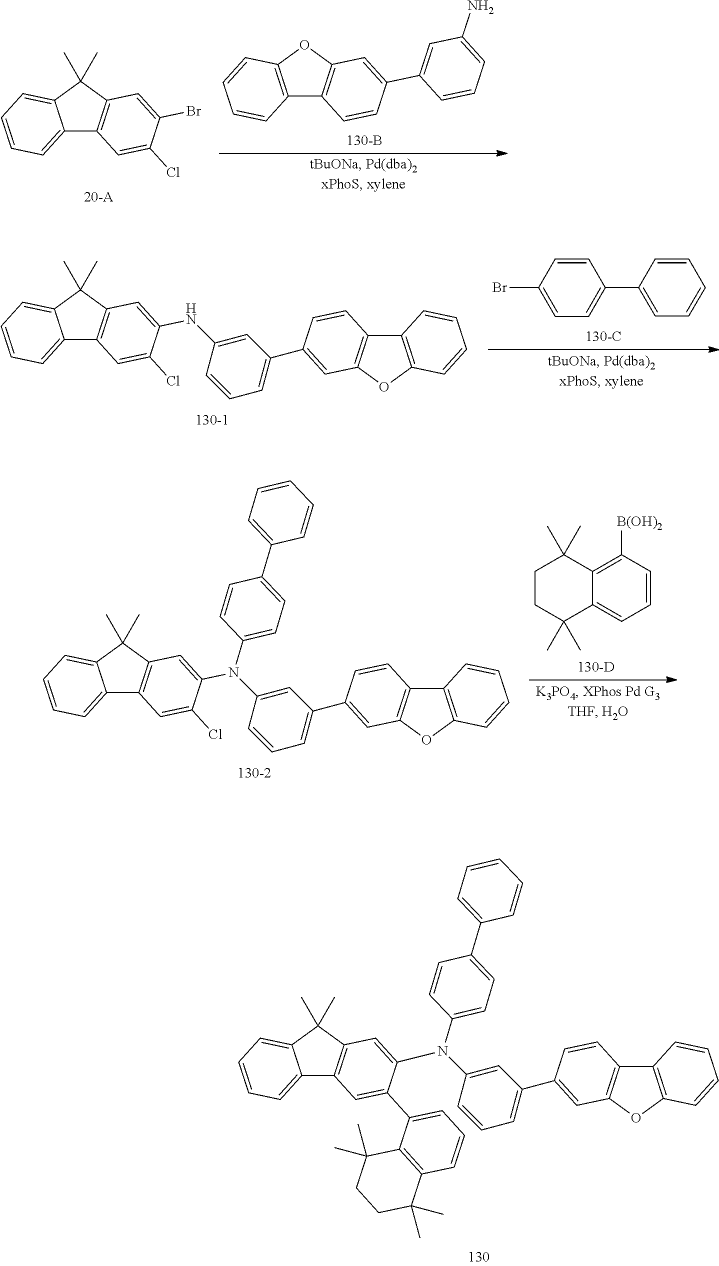

Except that the starting materials were replaced with 130-B, 130-C and 130-D, everything else was the same as Example 2. LC MS: M/Z 789.40 (M+). Total yield of synthesis: 49%; HPLC purity: 99.9%.

¹H NMR (500 MHz, DMSO-d6) δ 8.07-8.01 (m, 1H), 7.84 (d, 1H), 7.69-7.63 (m, 2H), 7.60-7.29 (m, 15H), 7.25 (t, 1H), 7.18-7.09 (m, 4H), 7.08 (s, 1H), 1.85-1.78 (m, 2H), 1.78-1.71 (m, 2H), 1.59 (s, 6H), 1.32 (s, 4H), 1.29 (s, 12H).

### Example 31

### Synthesis of Compound 137

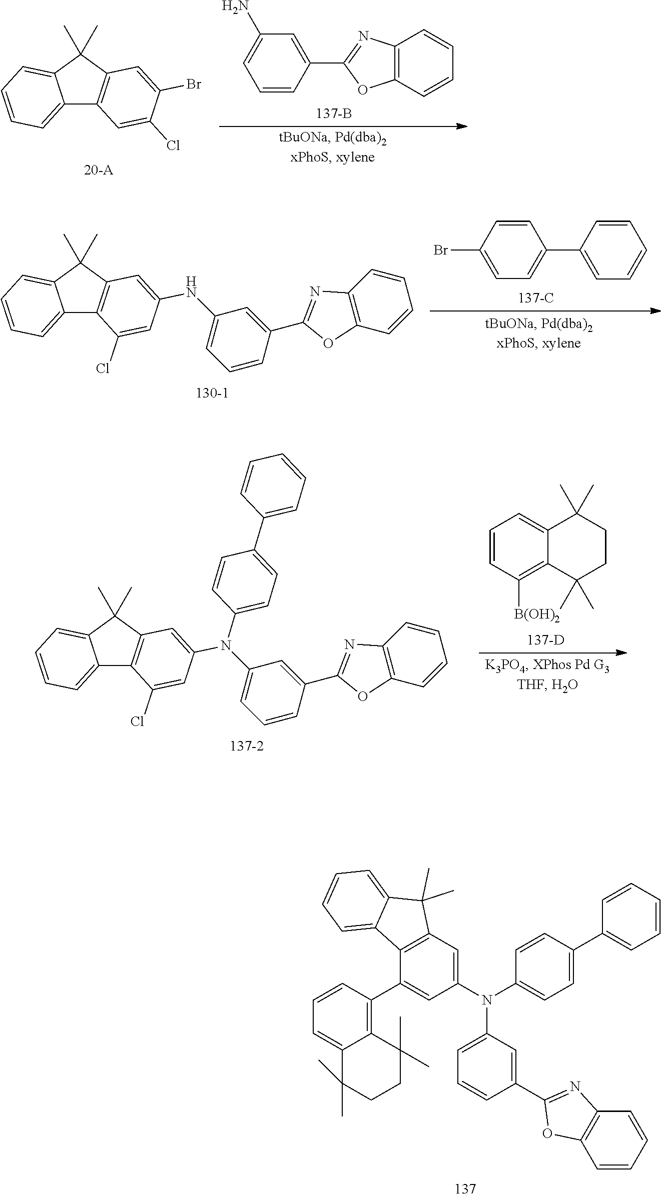

Except that the starting materials were replaced with 137-B, 137-C and 137-D, everything else was the same as Example 2. LC MS: M/Z 740.38 (M+). Total yield of synthesis: 48%; HPLC purity: 99.9%.

¹H NMR (500 MHz, DMSO-d6) δ 7.76 (m, 1H), 7.72-7.63 (m, 3H), 7.61 (t, 1H), 7.58-7.51 (m, 2H), 7.54-7.27 (m, 10H), 7.20-7.12 (m, 4H), 6.91 (d, 1H), 1.83-1.71 (m, 4H), 1.60 (s, 6H), 1.32 (s, 4H), 1.29 (s, 12H).

### Example 32

### Synthesis of Compound 139

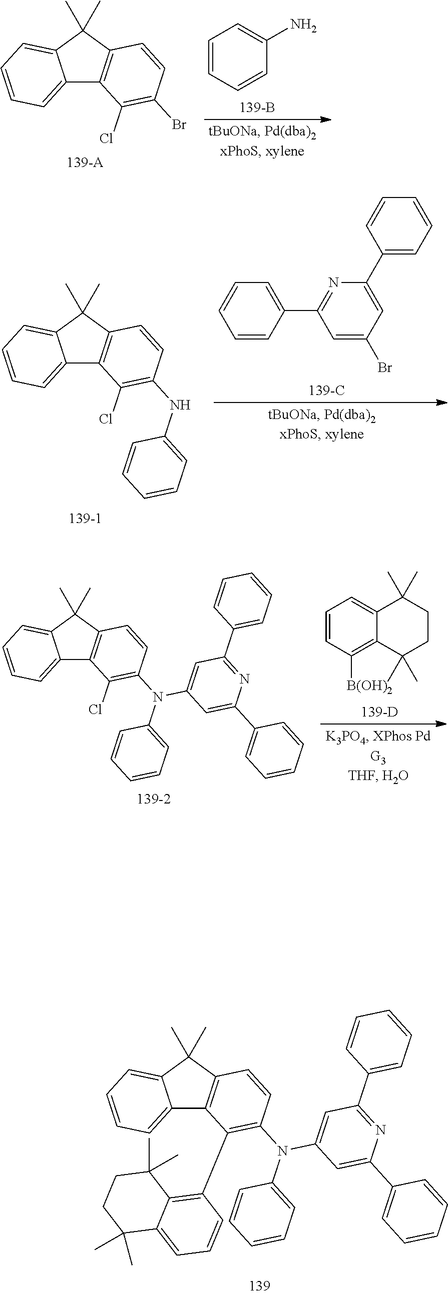

Except that the starting materials were replaced with 139-A, 139-B, 139-C and 139-D, everything else was the same as Example 2. LC MS: M/Z 700.38 (M+). Total yield of synthesis: 47%; HPLC purity: 99.9%.

¹H NMR (500 MHz, DMSO-d6) δ 8.17-8.10 (m, 4H), 7.75 (m, 1H), 7.56-7.48 (m, 4H), 7.48-7.24 (m, 8H), 7.17-7.04 (m, 5H), 1.81-1.70 (m, 4H), 1.59 (s, 6H), 1.31 (s, 4H), 1.28 (s, 12H).

### Example 33

### Synthesis of Compound 451

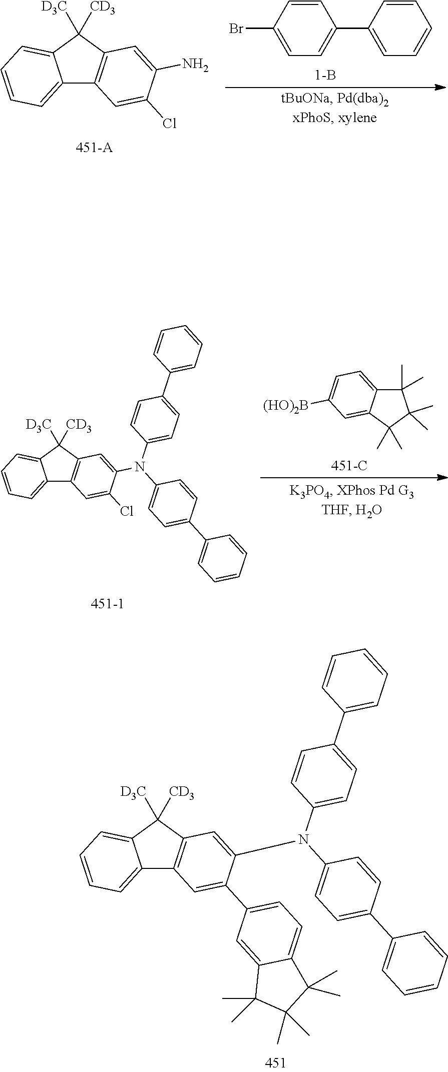 [SMILES: [2H]C([2H])([2H])C1(C([2H])([2H])[2H])c2ccccc2-c2cc(Cl)c(N)cc21 ; Brc1ccc(-c2ccccc2)cc1 ; [2H]C([2H])([2H])C1(C([2H])([2H])[2H])c2ccccc2-c2cc(Cl)c(N(c3ccc(-c4ccccc4)cc3)c3ccc(-c4ccccc4)cc3)cc21 ; CC1(C)c2ccc(B(O)O)cc2C(C)(C)C1(C)C ; [2H]C([2H])([2H])C1(C([2H])([2H])[2H])c2ccccc2-c2cc(-c3ccc4c(c3)C(C)(C)C(C)(C)C4(C)C)c(N(c3ccc(-c4ccccc4)cc3)c3ccc(-c4ccccc4)cc3)cc21]

Except that the starting materials were replaced with 451-A and 451-C, everything else was the same as Example 1. LC MS: M/Z 719.44 (M+). Total yield of synthesis: 59%; HPLC purity: 99.9%.

¹H NMR (500 MHz, DMSO-d6) δ 7.87 (s, 1H), 7.69-7.63 (m, 4H), 7.57-7.50 (m, 6H), 7.47 (m, 2H), 7.44-7.32 (m, 6H), 7.29 (m, 1H), 7.20-7.12 (m, 6H), 7.04 (d, 1H), 1.31 (s, 12H), 0.93 (s, 6H).

### Example 34

### Synthesis of Compound 452

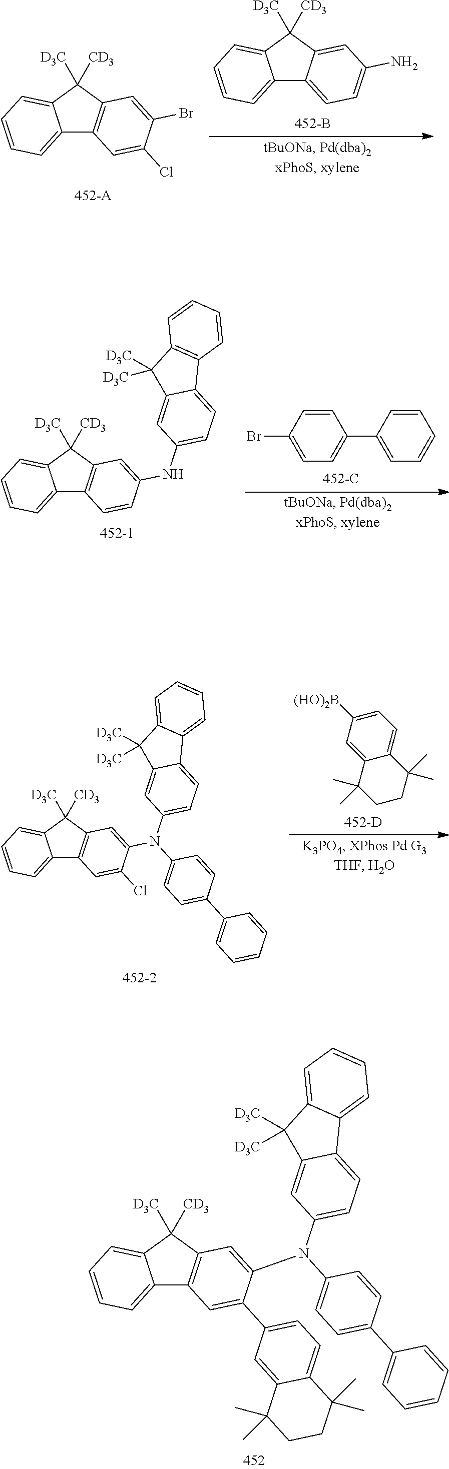

Except that the starting materials were replaced with 452-A, 452-B, 452-C and 452-D, everything else was the same as Example 2. LC MS: M/Z 892.40 (M+). Total yield of synthesis: 49%; HPLC purity: 99.9%.

¹H NMR (500 MHz, DMSO-d6) δ 7.86 (s, 1H), 7.72 (m, 1H), 7.69-7.61 (m, 3H), 7.58-7.50 (m, 3H), 7.49-7.27 (m, 11H), 7.25 (m, 1H), 7.22-7.12 (m, 4H), 6.99 (d, 1H), 1.74 (s, 3H), 1.74 (d, J=1H), 1.28 (d, 12H).

### Example 35

### Synthesis of Compound 456

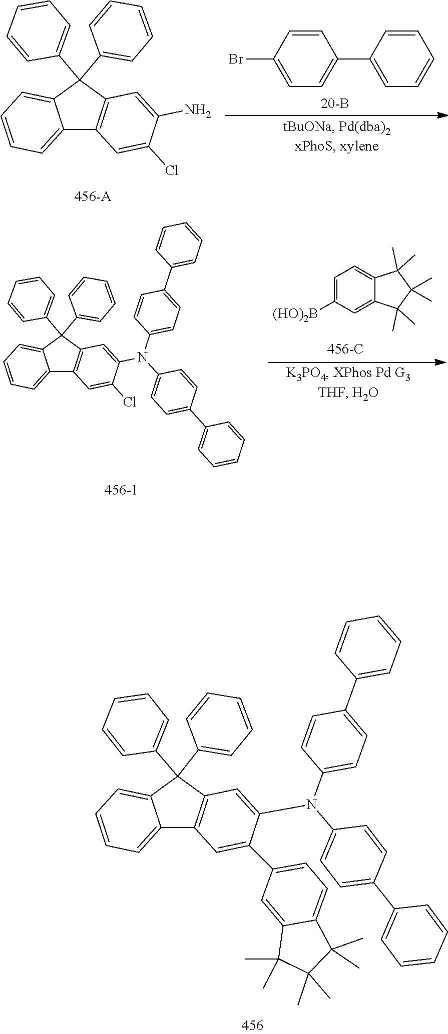 [SMILES: Nc1cc2c(cc1Cl)-c1ccccc1C2(c1ccccc1)c1ccccc1 ; Brc1ccc(-c2ccccc2)cc1 ; Clc1cc2c(cc1N(c1ccc(-c3ccccc3)cc1)c1ccc(-c3ccccc3)cc1)C(c1ccccc1)(c1ccccc1)c1ccccc1-2 ; CC1(C)c2ccc(B(O)O)cc2C(C)(C)C1(C)C ; CC1(C)c2ccc(-c3cc4c(cc3N(c3ccc(-c5ccccc5)cc3)c3ccc(-c5ccccc5)cc3)C(c3ccccc3)(c3ccccc3)c3ccccc3-4)cc2C(C)(C)C1(C)C]

Except that the starting materials were replaced with 456-A and 456-C, everything else was the same as Example 2. LC MS: M/Z 837.43 (M+). Total yield of synthesis: 46%; HPLC purity: 99.9%.

¹H NMR (500 MHz, DMSO-d6) δ 7.85 (s, 1H), 7.69-7.63 (m, 4H), 7.59 (m, 1H), 7.57-7.51 (m, 4H), 7.49-7.32 (m, 8H), 7.32-7.19 (m, 7H), 7.18-7.10 (m, 10H), 7.05-7.00 (m, 2H), 1.31 (s, 12H), 0.93 (s, 6H).

### Example 36

### Synthesis of Compound 457

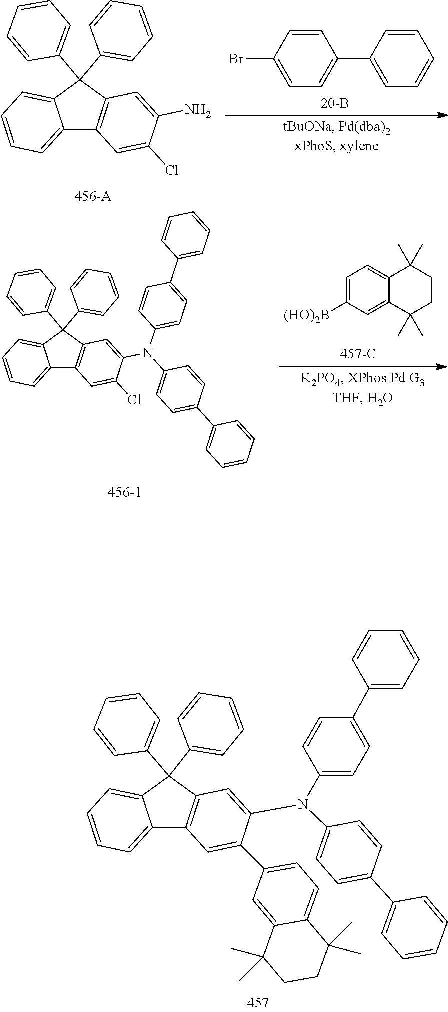 [SMILES: Nc1cc2c(cc1Cl)-c1ccccc1C2(c1ccccc1)c1ccccc1 ; Brc1ccc(-c2ccccc2)cc1 ; Clc1cc2c(cc1N(c1ccc(-c3ccccc3)cc1)c1ccc(-c3ccccc3)cc1)C(c1ccccc1)(c1ccccc1)c1ccccc1-2 ; CC1(C)CCC(C)(C)c2cc(B(O)O)ccc21 ; CC1(C)CCC(C)(C)c2cc(-c3cc4c(cc3N(c3ccc(-c5ccccc5)cc3)c3ccc(-c5ccccc5)cc3)C(c3ccccc3)(c3ccccc3)c3ccccc3-4)ccc21]

Except that the starting materials were replaced with 456-A and 457-C, everything else was the same as Example 2. LC MS: M/Z 823.42 (M+). Total yield of synthesis: 49%; HPLC purity: 99.9%.

¹H NMR (500 MHz, DMSO-d6) δ 7.85 (s, 1H), 7.69-7.63 (m, 4H), 7.59 (m, 1H), 7.57-7.51 (m, 4H), 7.46-7.37 (m, 6H), 7.40-7.32 (m, 4H), 7.32-7.09 (m, 13H), 7.02 (s, 1H), 1.74 (s, 3H), 1.38 (s, 4H), 0.98 (s, 12H).

### Example 37

### Synthesis of Compound 458

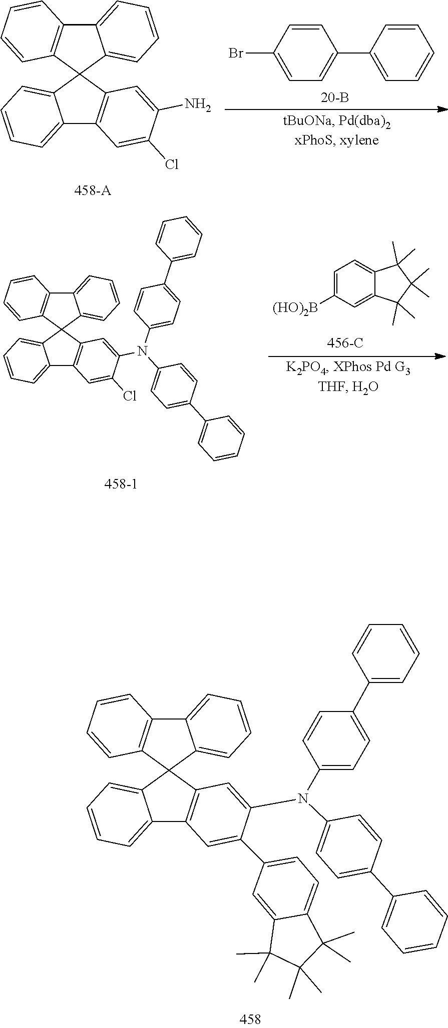 [SMILES: Nc1cc2c(cc1Cl)-c1ccccc1C21c2ccccc2-c2ccccc21 ; Brc1ccc(-c2ccccc2)cc1 ; Clc1cc2c(cc1N(c1ccc(-c3ccccc3)cc1)c1ccc(-c3ccccc3)cc1)C1(c3ccccc3-c3ccccc31)c1ccccc1-2 ; CC1(C)c2ccc(B(O)O)cc2C(C)(C)C1(C)C ; CC1(C)c2ccc(-c3cc4c(cc3N(c3ccc(-c5ccccc5)cc3)c3ccc(-c5ccccc5)cc3)C3(c5ccccc5-c5ccccc53)c3ccccc3-4)cc2C(C)(C)C1(C)C]

Except that the starting materials were replaced with 458-A and 456-C, everything else was the same as Example 2. LC MS: M/Z 892.40 (M+). Total yield of synthesis: 49%; HPLC purity: 99.9%.

¹H NMR (500 MHz, DMSO-d6) δ 7.84 (m, 2H), 7.81 (s, 1H), 7.69-7.63 (m, 4H), 7.59 (m, 1H), 7.57-7.51 (m, 4H), 7.46 (m, 1H), 7.44-7.25 (m, 12H), 7.18-7.12 (m, 5H), 7.06-7.01 (m, 2H), 6.87 (m, 1H), 6.83-6.78 (m, 2H), 1.31 (s, 12H), 0.93 (s, 6H).

### Example 38

### Synthesis of Compound 461

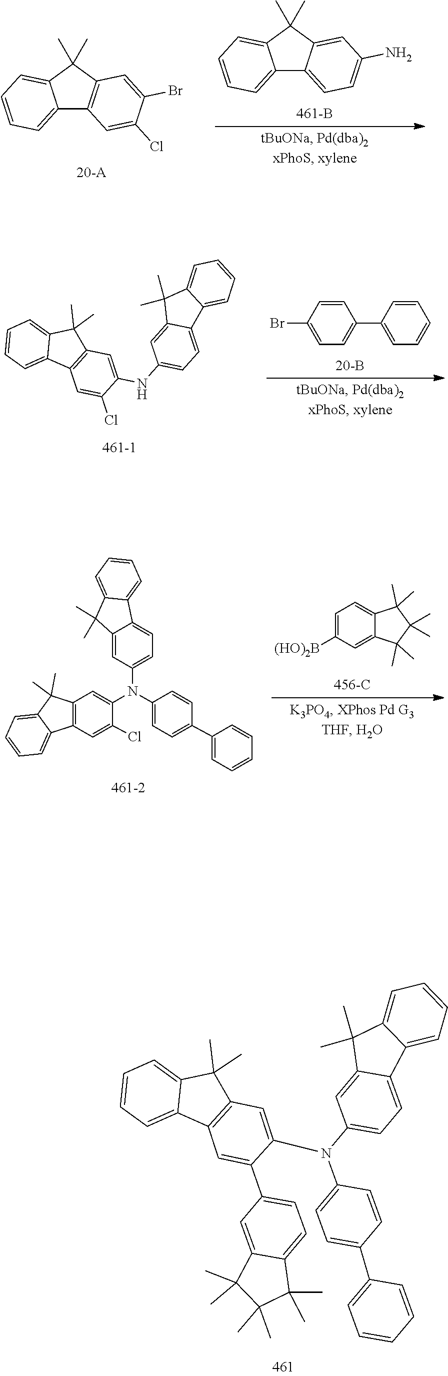

Except that the starting materials were replaced with 461-B and 456-C, everything else was the same as Example 2. LC MS: M/Z 753.43 (M+). Total yield of synthesis: 48%; HPLC purity: 99.9%.

¹H NMR (500 MHz, DMSO-d6) δ 7.87 (s, 1H), 7.75-7.63 (m, 4H), 7.57-7.50 (m, 3H), 7.47 (m, 3H), 7.44-7.39 (m, 1H), 7.42-7.30 (m, 6H), 7.23 (m, 1H), 7.18-7.12 (m, 3H), 7.09 (s, 1H), 7.04 (d, 1H), 6.97 (d, 1H), 1.59 (d, 12H), 1.31 (s, 12H), 0.93 (s, 6H).

### Example 39

### Synthesis of Compound 462

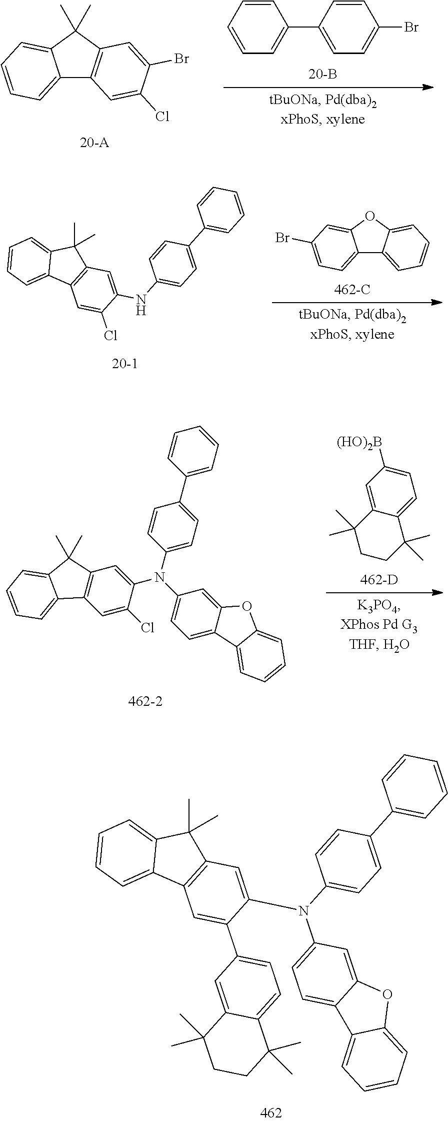

Except that the starting materials were replaced with 462-C and 462-D, everything else was the same as Example 2. LC MS: M/Z 713.37 (M+). Total yield of synthesis: 46%; HPLC purity: 99.9%.

¹H NMR (500 MHz, DMSO-d6) δ 8.01 (m, 1H), 7.94 (d, 1H), 7.85 (s, 1H), 7.69-7.63 (m, 2H), 7.58-7.50 (m, 4H), 7.52-7.29 (m, 10H), 7.22-7.12 (m, 3H), 7.11-7.05 (m, 2H), 6.88 (m, 1H), 1.74 (s, 4H), 1.59 (s, 6H), 1.28 (d, 12H).

### Example 40

### Synthesis of Compound 464

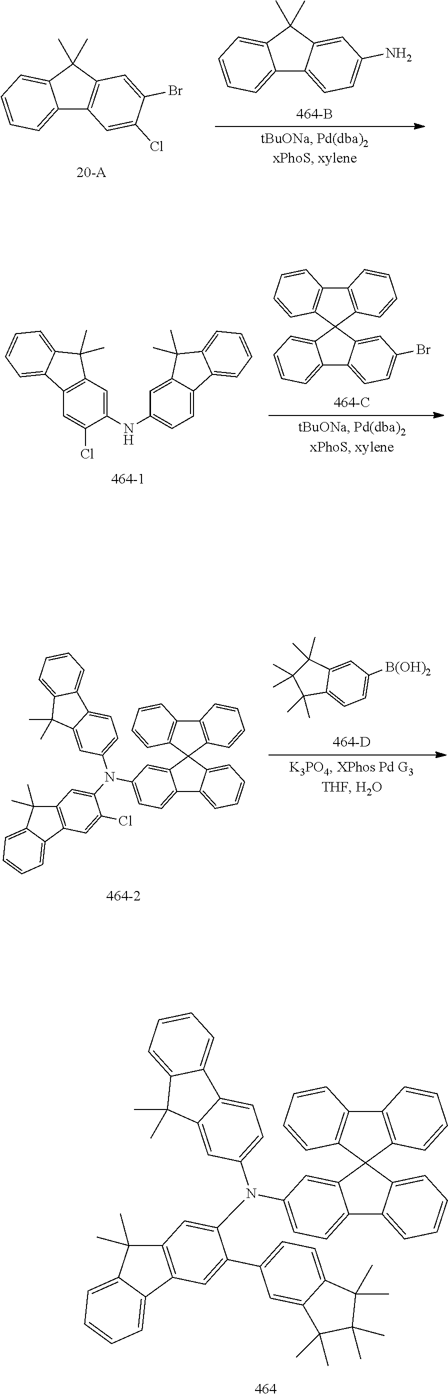

Except that the starting materials were replaced with 464-B, 464-C and 464-D, everything else was the same as Example 2. LC MS: M/Z 915.48 (M+). Total yield of synthesis: 46%; HPLC purity: 99.9%.

¹H NMR (500 MHz, DMSO-d6) δ 7.89-7.82 (m, 4H), 7.75-7.66 (m, 3H), 7.53 (m, 1H), 7.49-7.43 (m, 3H), 7.40-7.20 (m, 12H), 7.15 (d, 1H), 7.09 (s, 1H), 7.03 (d, 1H), 6.95 (d, 1H), 6.85 (d, 1H), 6.81 (m, 1H), 6.75 (m, 2H), 1.59 (s, 12H), 1.31 (s, 12H), 0.93 (s, 6H).

### Example 41

### Synthesis of Compound 465

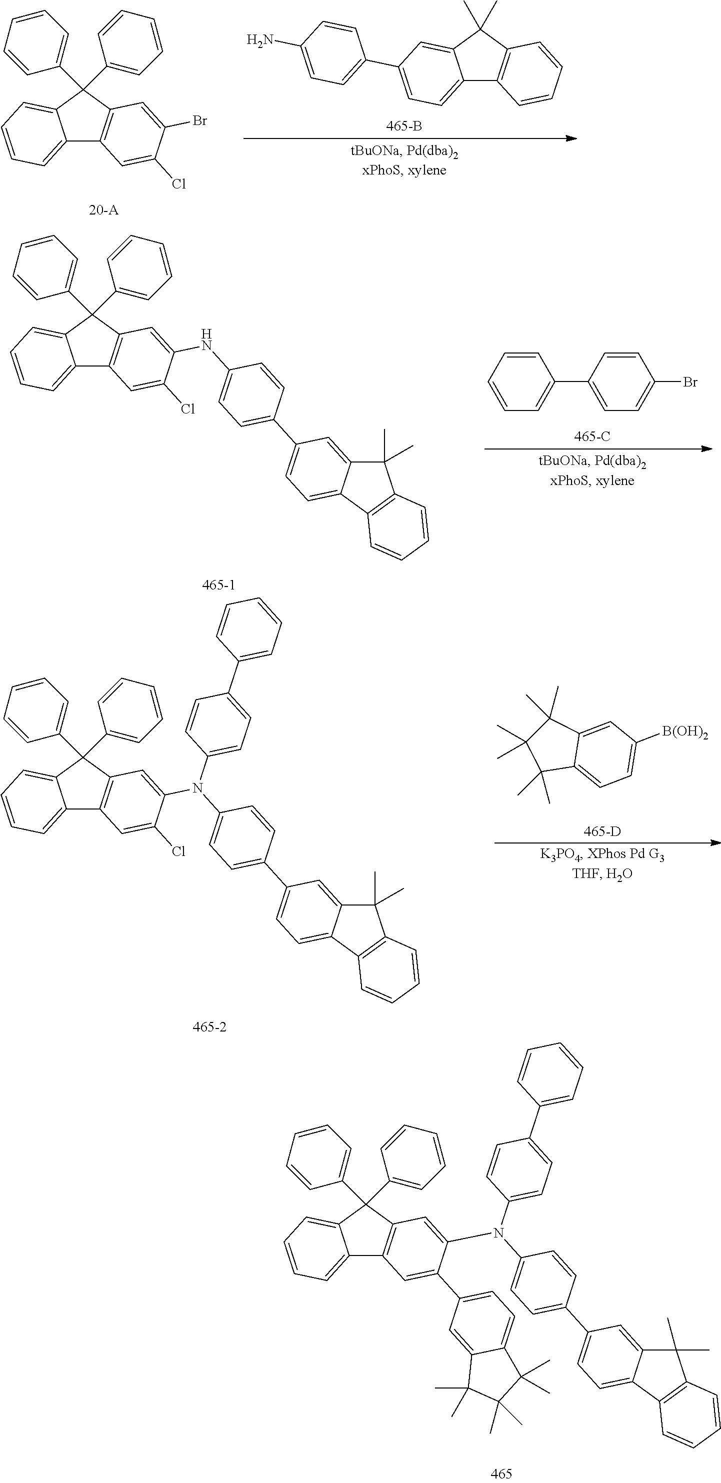

Except that the starting materials were replaced with 465-B, 465-C and 465-D, everything else was the same as Example 2. LC MS: M/Z 953.50 (M+). Total yield of synthesis: 49%; HPLC purity: 99.9%.

¹H NMR (500 MHz, DMSO-d6) δ 7.85 (s, 1H), 7.81 (d, 1H), 7.72 (m, 1H), 7.69-7.63 (m, 2H), 7.63-7.51 (m, 9H), 7.46 (m, 1H), 7.45-7.40 (m, 4H), 7.43-7.35 (m, 6H), 7.38-7.31 (m, 4H), 7.33-7.25 (m, 5H), 7.28-7.19 (m, 3H), 7.21-7.13 (m, 5H), 7.16-7.10 (m, 5H), 7.05-7.00 (m, 2H), 1.59 (s, 6H), 1.31 (s, 12H), 0.93 (s, 6H).

### Example 42

### Synthesis of Compound 467

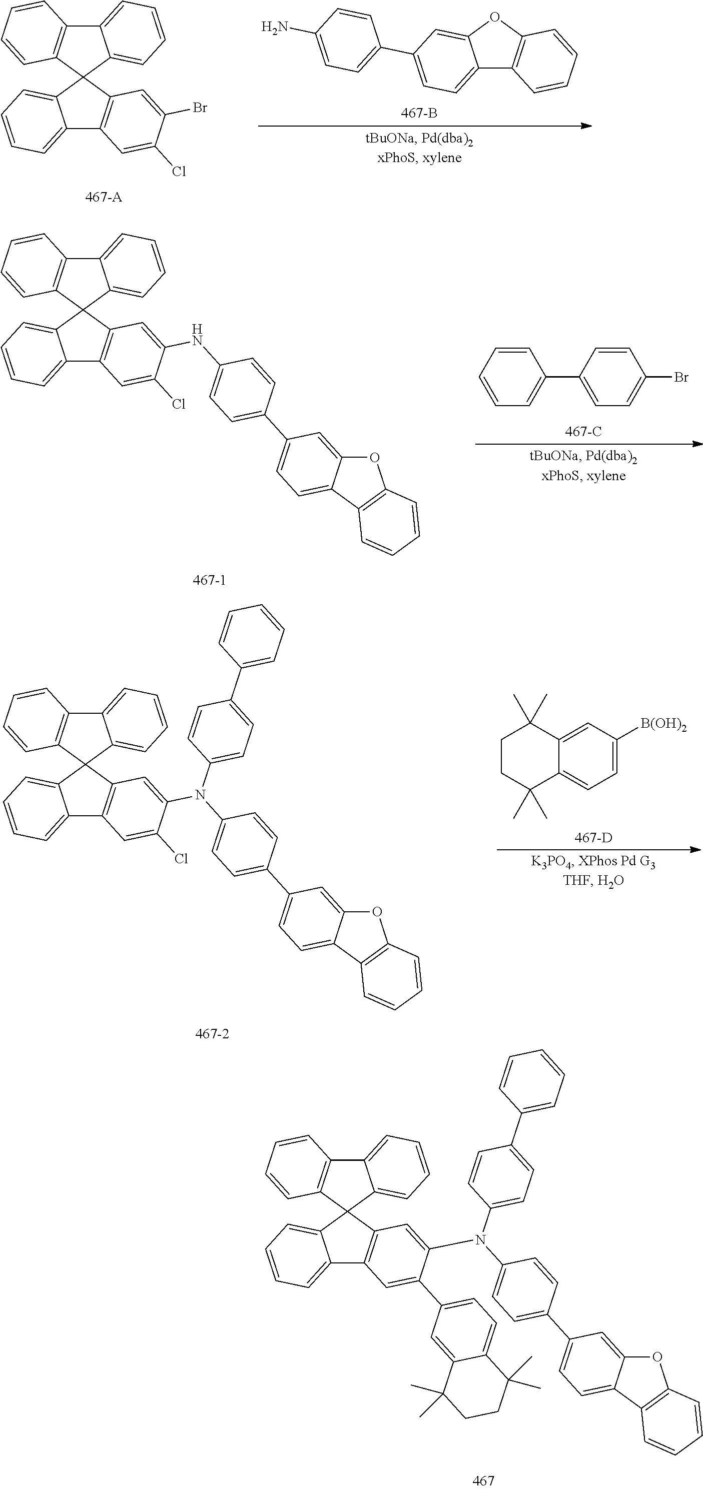

Except that the starting materials were replaced with 467-A, 467-B, 467-C and 467-D, everything else was the same as Example 2. LC MS: M/Z 911.41 (M+). Total yield of synthesis: 46%; HPLC purity: 99.9%.

¹H NMR (500 MHz, DMSO-d6) δ 8.06-8.00 (m, 1H), 7.89 (d, 1H), 7.84 (m, 2H), 7.81 (s, 1H), 7.73 (d, 1H), 7.69-7.63 (m, 2H), 7.65-7.59 (m, 2H), 7.63-7.44 (m, 6H), 7.48-7.33 (m, 6H), 7.36-7.25 (m, 6H), 7.19 (d, 1H), 7.17-7.12 (m, 4H), 7.04 (s, 1H), 6.87 (m, 1H), 6.84-6.77 (m, 2H), 1.74 (s, 4H), 1.28 (s, 12H).

### Example 43

### Synthesis of Compound 469

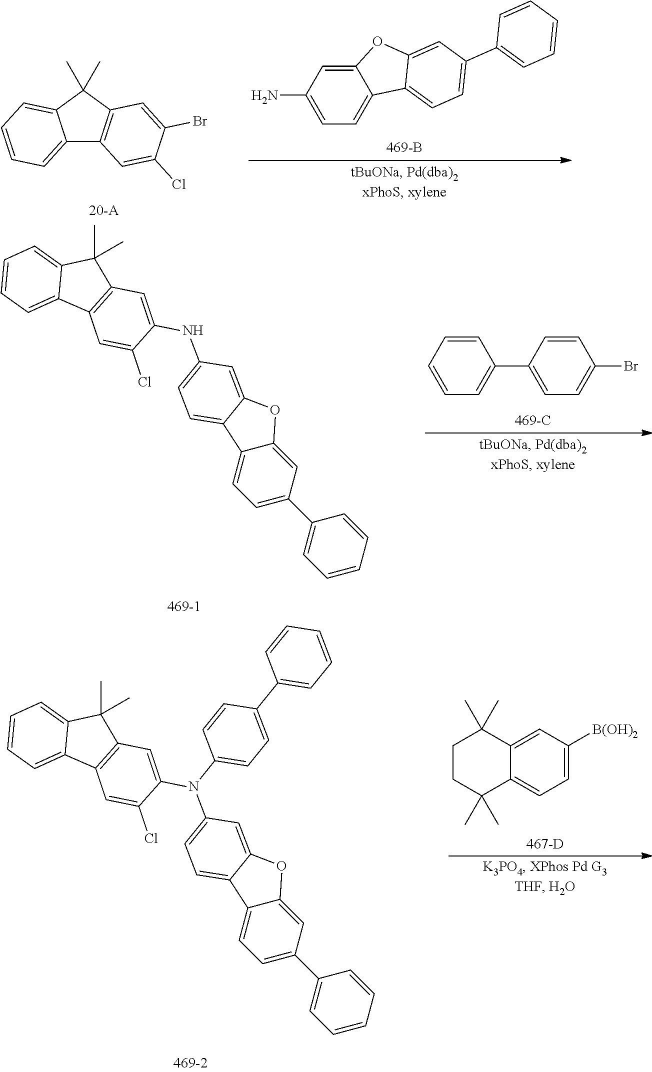 [SMILES: CC1(C)c2ccccc2-c2cc(Cl)c(Br)cc21 ; Nc1ccc2c(c1)oc1cc(-c3ccccc3)ccc12 ; CC1(C)c2ccccc2-c2cc(Cl)c(Nc3ccc4c(c3)oc3cc(-c5ccccc5)ccc34)cc21 ; Brc1ccc(-c2ccccc2)cc1 ; CC1(C)c2ccccc2-c2cc(Cl)c(N(c3ccc(-c4ccccc4)cc3)c3ccc4c(c3)oc3cc(-c5ccccc5)ccc34)cc21 ; CC1(C)CCC(C)(C)c2cc(B(O)O)ccc21]

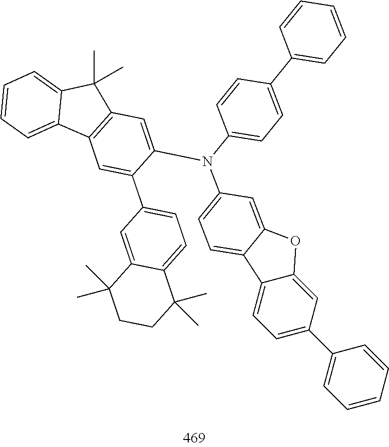 [SMILES: CC1(C)CCC(C)(C)c2cc(-c3cc4c(cc3N(c3ccc(-c5ccccc5)cc3)c3ccc5c(c3)oc3cc(-c6ccccc6)ccc35)C(C)(C)c3ccccc3-4)ccc21]

Except that the starting materials were replaced with 469-B, 469-C and 467-D, everything else was the same as Example 2. LC MS: M/Z 892.40 (M+). Total yield of synthesis: 49%; HPLC purity: 99.9%.

¹H NMR (500 MHz, DMSO-d6) δ 7.92 (d, 1H), 7.89-7.82 (m, 2H), 7.73 (d, 1H), 7.69-7.57 (m, 5H), 7.57-7.50 (m, 3H), 7.50-7.28 (m, 11H), 7.22-7.12 (m, 3H), 7.16-7.07 (m, 2H), 6.89 (m, 1H), 1.74 (s, 3H), 1.74 (d, 1H), 1.59 (s, 6H), 1.28 (s, 12H).

### Example 44

### Synthesis of Compound 470

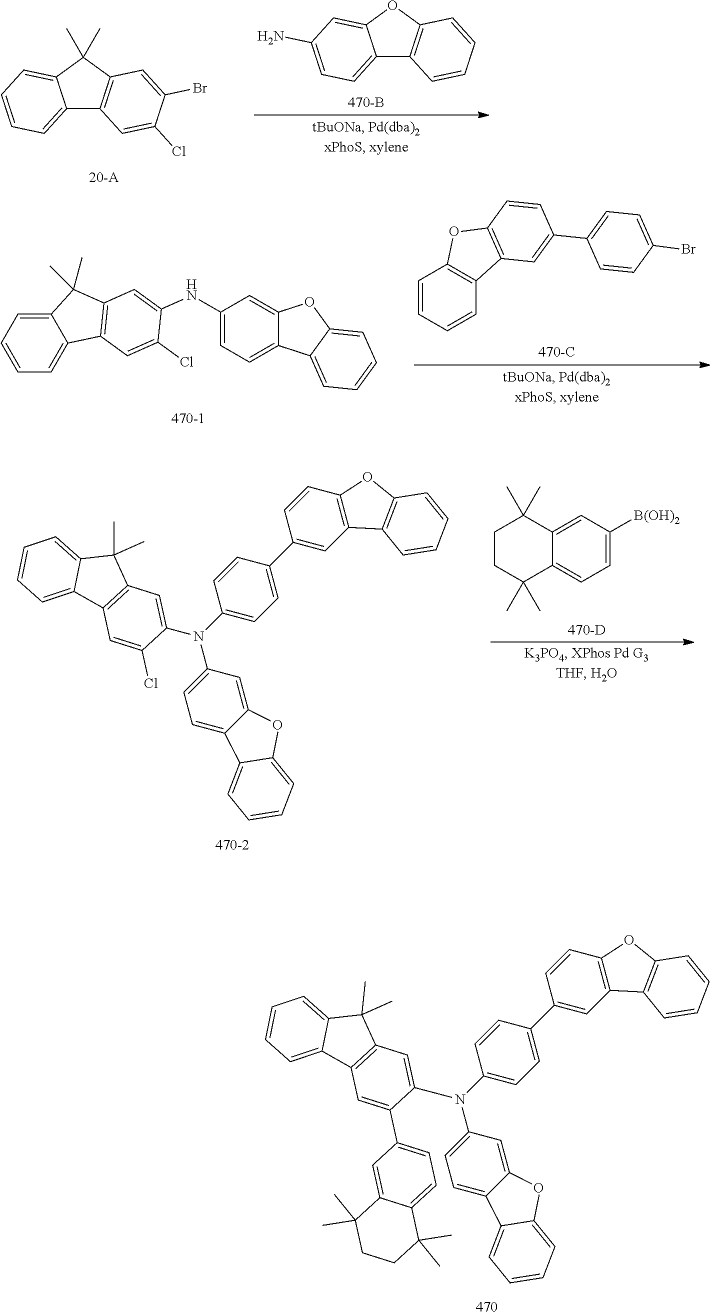

Except that the starting materials were replaced with 470-B, 470-C and 470-D, everything else was the same as Example 2. LC MS: M/Z 803.38 (M+). Total yield of synthesis: 47%; HPLC purity: 99.9%.

¹H NMR (500 MHz, DMSO-d6) δ 8.07-8.00 (m, 2H), 7.92 (d, 1H), 7.90-7.85 (m, 2H), 7.67-7.59 (m, 3H), 7.59-7.51 (m, 3H), 7.54-7.47 (m, 1H), 7.51-7.40 (m, 6H), 7.40-7.33 (m, 2H), 7.32 (m, 1H), 7.22-7.13 (m, 3H), 7.11-7.05 (m, 2H), 6.88 (m, 1H), 1.74 (s, 3H), 1.74 (d, 1H), 1.59 (s, 6H), 1.28 (s, 12H).

### Example 45

### Synthesis of Compound 471

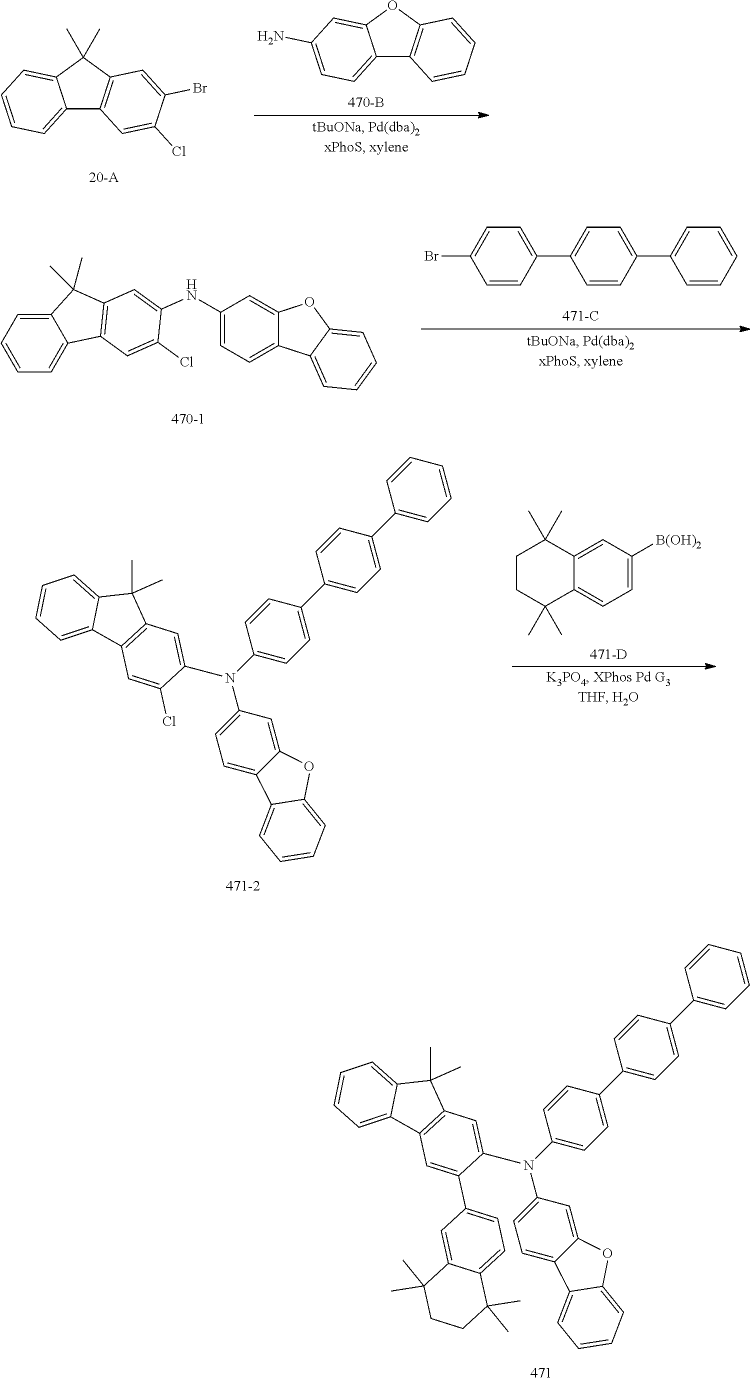

Except that the starting materials were replaced with 470-B, 471-C and 471-D, everything else was the same as Example 2. LC MS: M/Z 803.38 (M+). Total yield of synthesis: 47%; HPLC purity: 99.9%.

¹H NMR (500 MHz, DMSO-d6) δ 8.03 (m, 1H), 7.92 (d, 1H), 7.87 (s, 1H), 7.69-7.59 (m, 2H), 7.63 (s, 4H), 7.58-7.51 (m, 4H), 7.51-7.28 (m, 10H), 7.22-7.12 (m, 3H), 7.11-7.05 (m, 2H), 6.88 (m, 1H), 1.74 (s, 3H), 1.74 (d, 1H), 1.59 (s, 6H), 1.28 (s, 12H).

### Example 46

### Synthesis of Compound 473

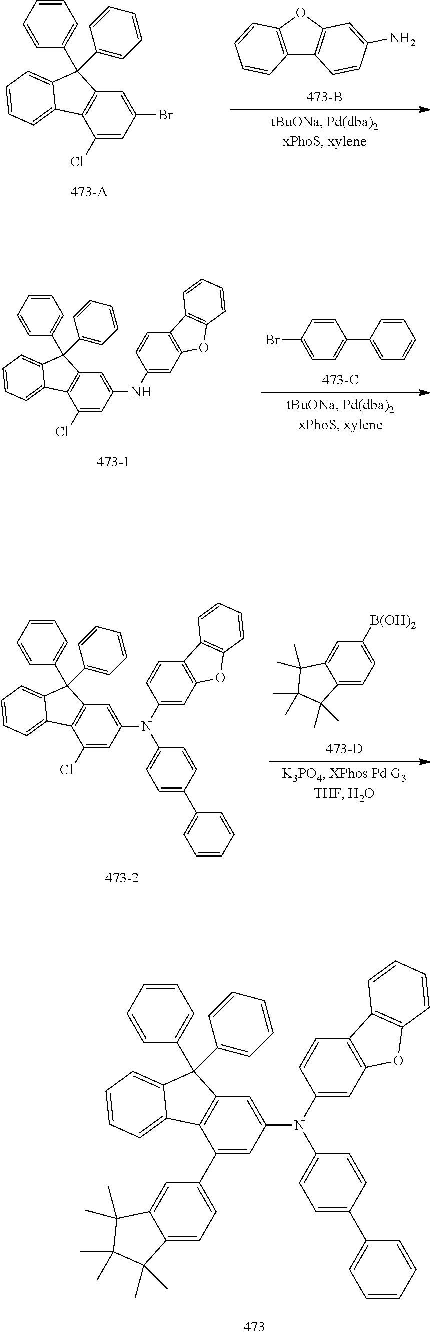

Except that the starting materials were replaced with 473-A, 473-B, 473-C and 473-D, everything else was the same as Example 2. LC MS: M/Z 851.41 (M+). Total yield of synthesis: 46%; HPLC purity: 99.9%.

¹H NMR (500 MHz, DMSO-d6) δ 8.01 (m, 1H), 7.92 (d, 1H), 7.73 (m, 1H), 7.69-7.63 (m, 2H), 7.59-7.52 (m, 3H), 7.51-7.32 (m, 8H), 7.32-7.20 (m, 8H), 7.20-7.13 (m, 4H), 7.13-7.06 (m, 5H), 7.03 (d, 1H), 6.87 (m, 1H), 1.31 (s, 12H), 0.93 (s, 6H).

### Example 47

### Synthesis of Compound 475

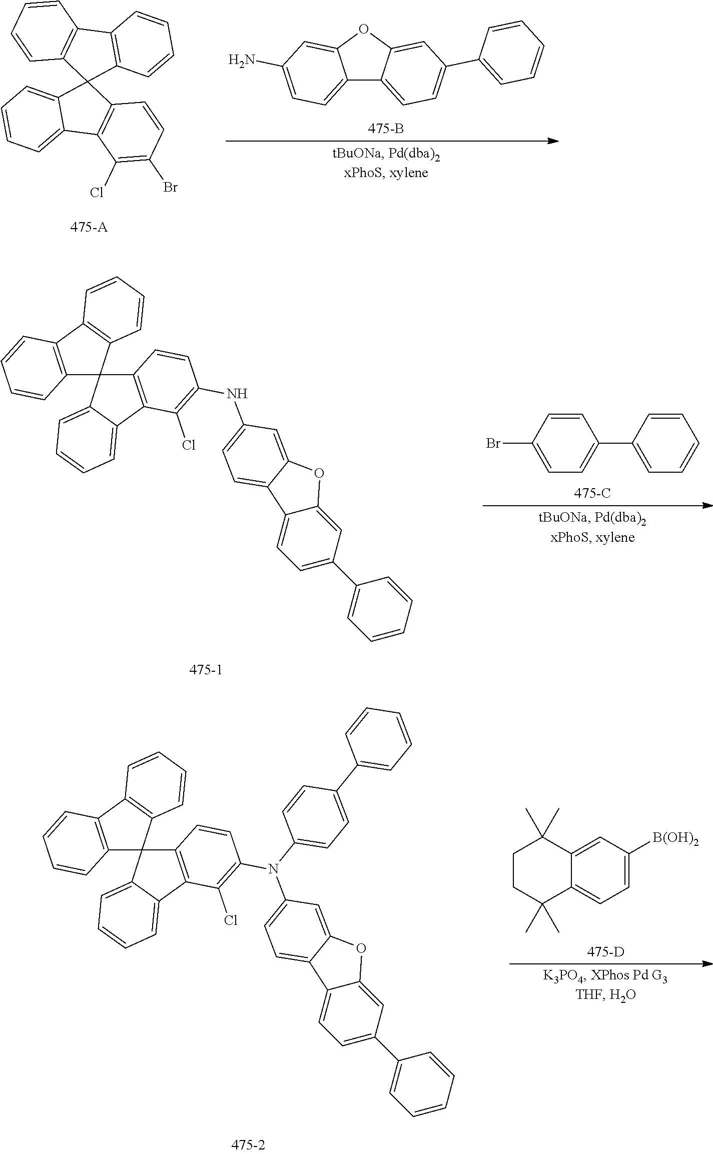 [SMILES: Clc1c(Br)ccc2c1-c1ccccc1C21c2ccccc2-c2ccccc21 ; Nc1ccc2c(c1)oc1cc(-c3ccccc3)ccc12 ; Clc1c(Nc2ccc3c(c2)oc2cc(-c4ccccc4)ccc23)ccc2c1-c1ccccc1C21c2ccccc2-c2ccccc21 ; Brc1ccc(-c2ccccc2)cc1 ; Clc1c(N(c2ccc(-c3ccccc3)cc2)c2ccc3c(c2)oc2cc(-c4ccccc4)ccc23)ccc2c1-c1ccccc1C21c2ccccc2-c2ccccc21 ; CC1(C)CCC(C)(C)c2cc(B(O)O)ccc21]

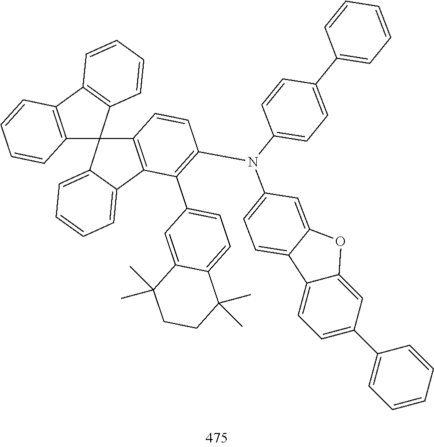 [SMILES: CC1(C)CCC(C)(C)c2cc(-c3c(N(c4ccc(-c5ccccc5)cc4)c4ccc5c(c4)oc4cc(-c6ccccc6)ccc45)ccc4c3-c3ccccc3C43c4ccccc4-c4ccccc43)ccc21]

Except that the starting materials were replaced with 475-A, 475-B, 475-C and 475-D, everything else was the same as Example 2. LC MS: M/Z 911.41 (M+). Total yield of synthesis: 45%; HPLC purity: 99.9%.

¹H NMR (500 MHz, DMSO-d6) δ 7.92 (d, 1H), 7.84 (m, 3H), 7.73 (d, 1H), 7.71-7.59 (m, 5H), 7.59-7.51 (m, 3H), 7.49 (m, 1H), 7.45-7.35 (m, 6H), 7.39-7.30 (m, 4H), 7.33-7.23 (m, 6H), 7.21 (d, 1H), 7.18-7.09 (m, 3H), 6.89 (m, 1H), 6.81 (m, 2H), 1.74 (s, 3H), 1.74 (d, 1H), 1.28 (d, 12H).

### Example 48

### Synthesis of Compound 477

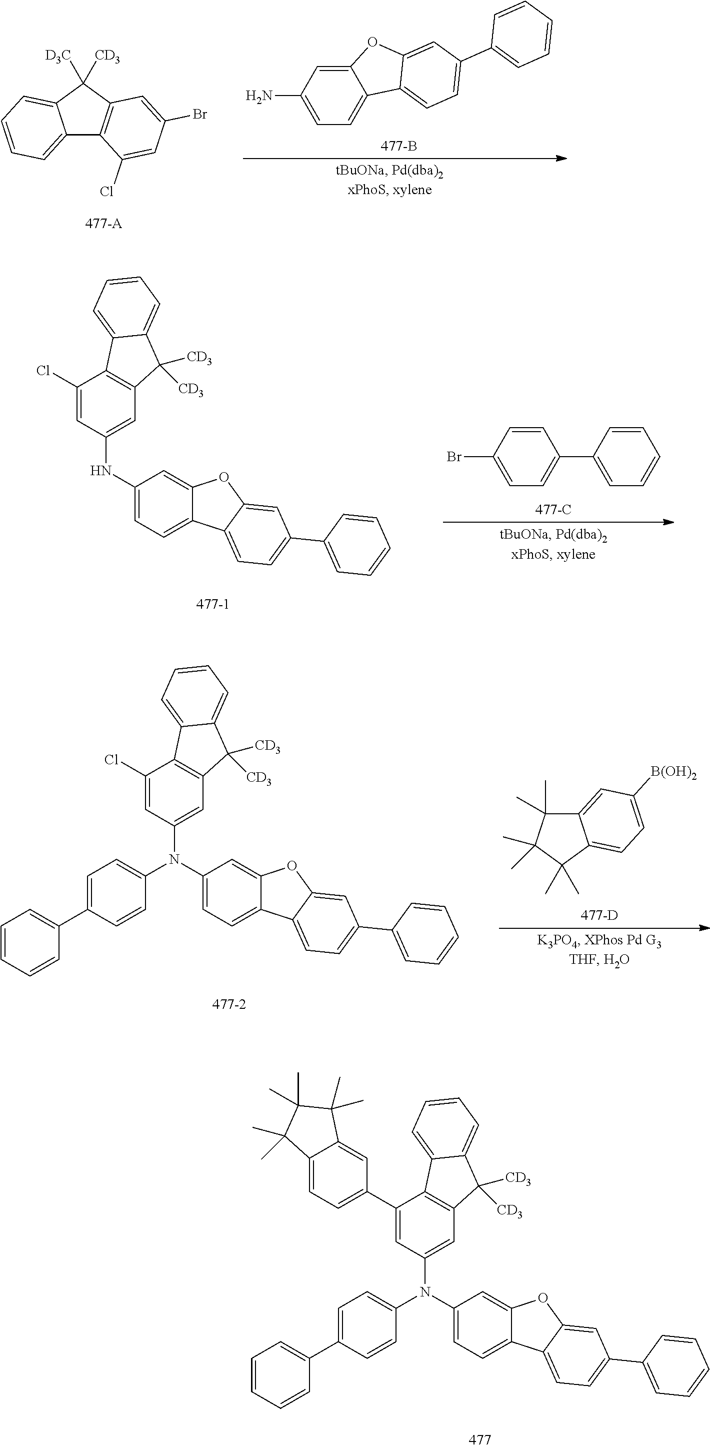

Except that the starting materials were replaced with 477-A, 477-B, 477-C and 477-D, everything else was the same as Example 2. LC MS: M/Z 809.45 (M+). Total yield of synthesis: 48%; HPLC purity: 99.9%.

¹H NMR (500 MHz, DMSO-d6) δ 7.93 (d, 1H), 7.84 (d, 1H), 7.77-7.71 (m, 2H), 7.69-7.54 (m, 8H), 7.49-7.42 (m, 3H), 7.45-7.39 (m, 1H), 7.42-7.35 (m, 4H), 7.38-7.30 (m, 1H), 7.32-7.25 (m, 2H), 7.17 (m, 2H), 7.09 (d, 1H), 7.04 (d, 1H), 6.95 (d, 1H), 6.88 (m, 1H), 1.31 (s, 12H), 0.93 (s, 6H).

### Example 49

### Synthesis of Compound 3

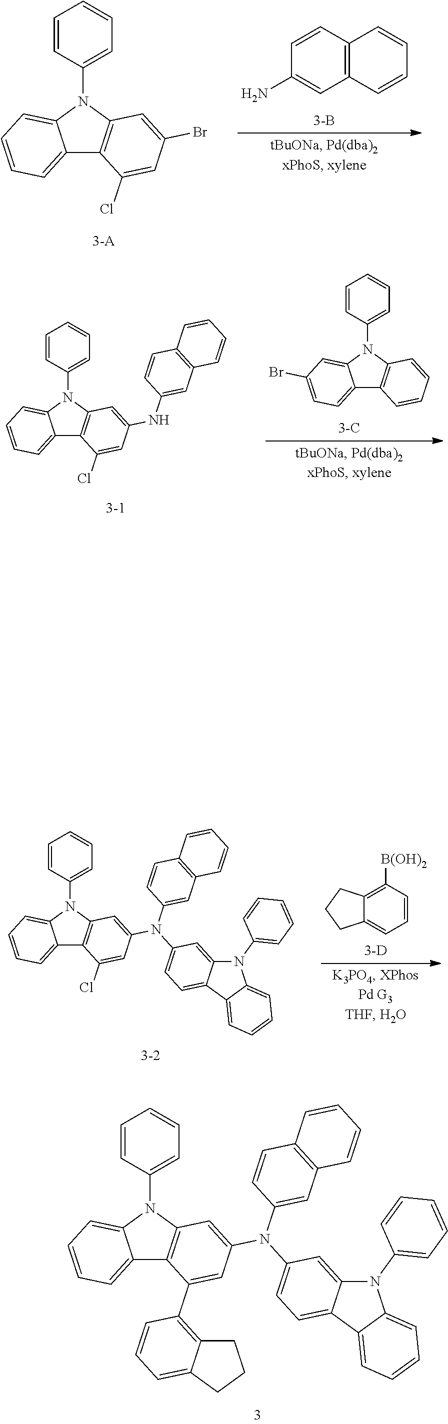

Except that the starting materials were replaced with 3-A, 3-B, 3-C and 3-D, everything else was the same as Example 2. LC MS: M/Z 741.31 (M+). Total yield of synthesis: 47%; HPLC purity: 99.9%.

¹H NMR (500 MHz, DMSO-d6) δ 8.10 (m, 1H), 8.05 (d, 1H), 7.98-7.90 (m, 1H), 7.80 (m, 2H), 7.65-7.40 (m, 16H), 7.38-7.16 (m, 11H), 7.11 (d, 1H), 2.92-2.85 (m, 2H), 2.72 (t, 2H), 2.17 (m, 2H).

### Example 50

### Synthesis of Compound 6

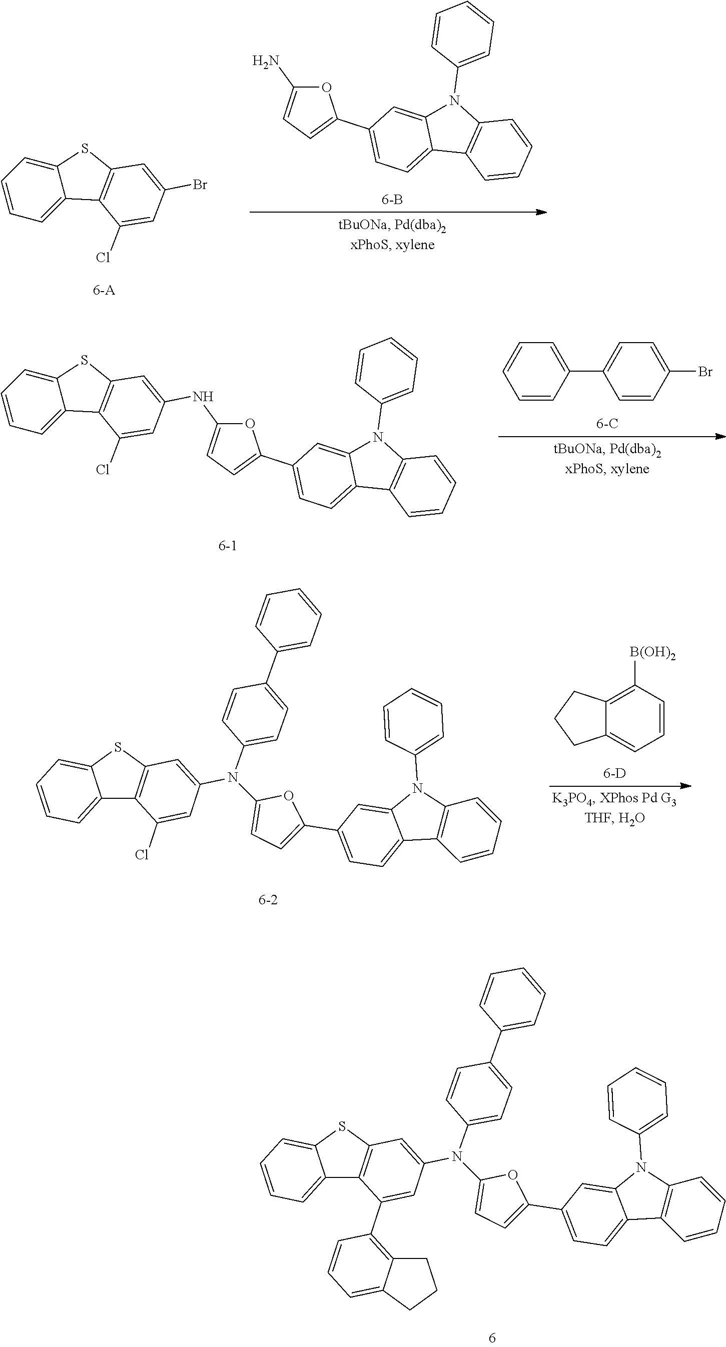

Except that the starting materials were replaced with 6-A, 6-B, 6-C and 6-D, everything else was the same as Example 2. LC MS: M/Z 774.27 (M+). Total yield of synthesis: 46%; HPLC purity: 99.9%.

### Example 51

### Synthesis of Compound 28

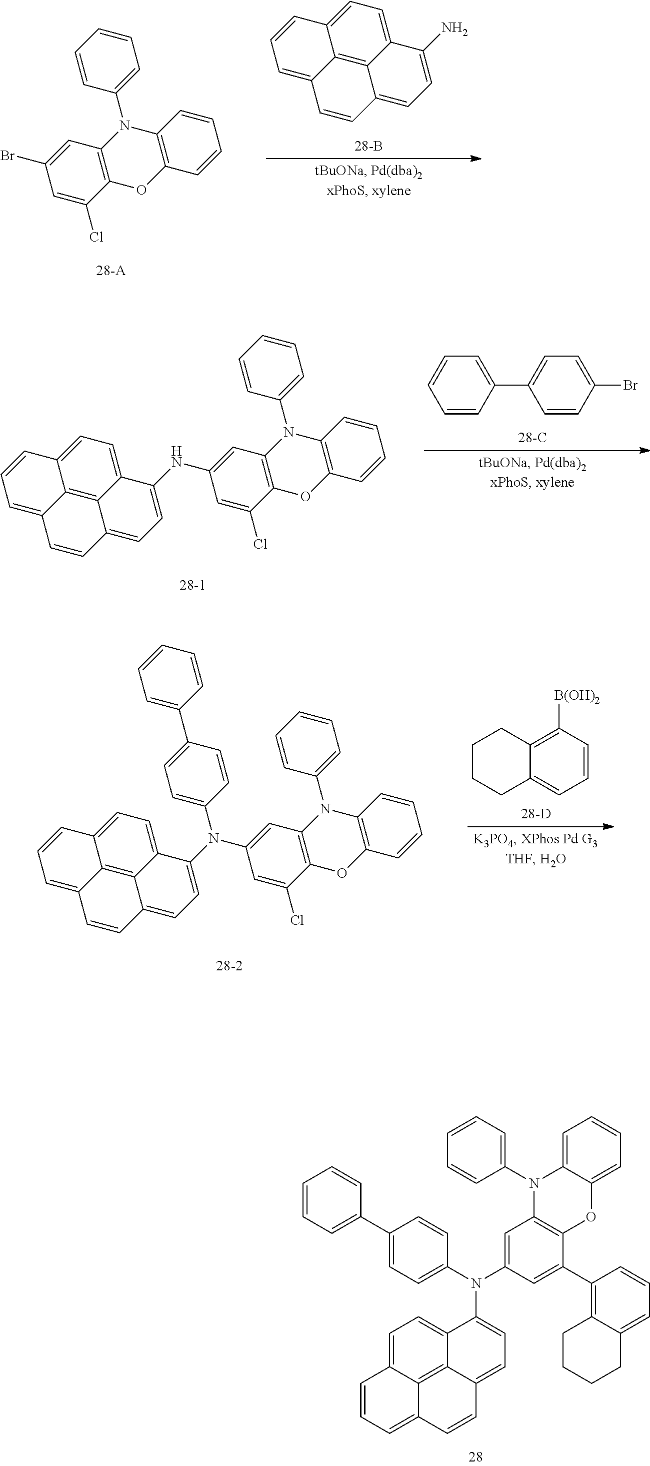

Except that the starting materials were replaced with 28-A, 28-B, 28-C and 28-D, everything else was the same as Example 2. LC MS: M/Z 809.45 (M+). Total yield of synthesis: 48%; HPLC purity: 99.9%.

### Example 52

### Synthesis of Compound 43

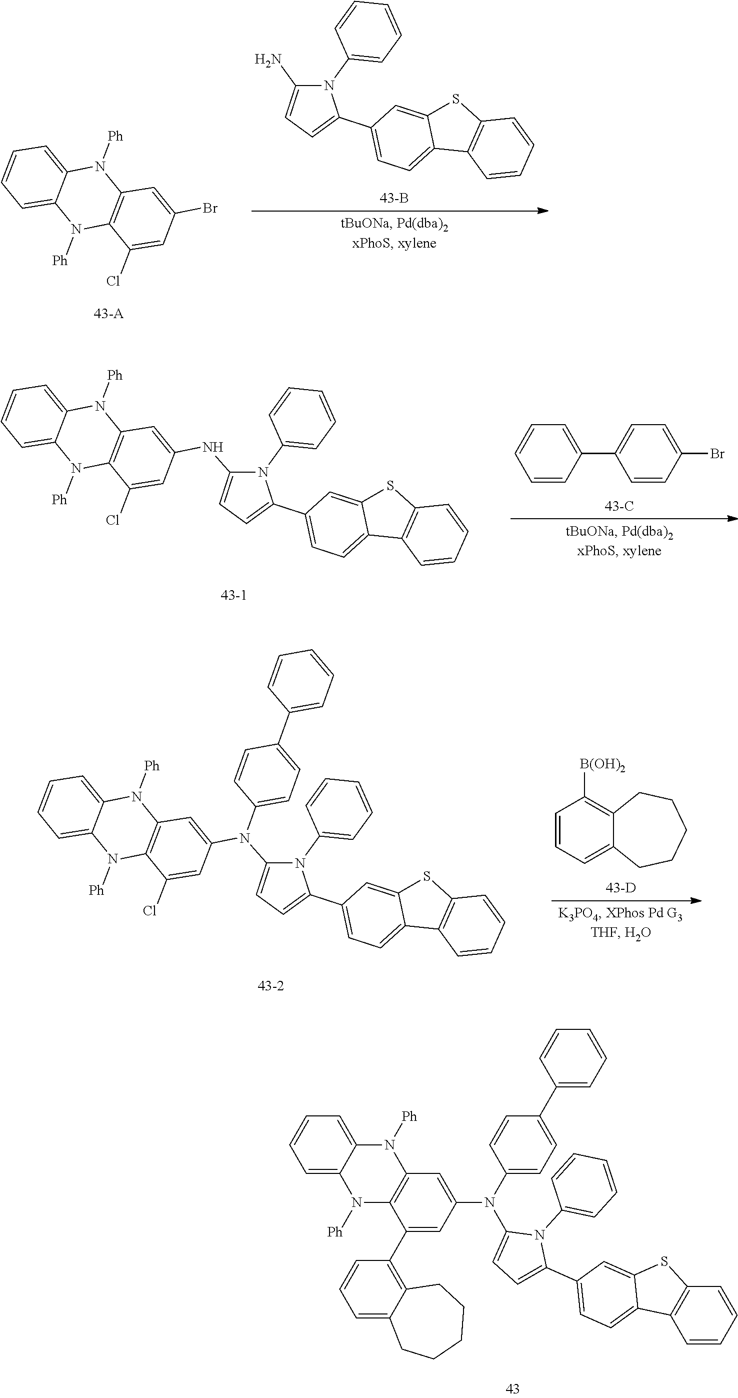

Except that the starting materials were replaced with 43-A, 43-B, 43-C and 43-D, everything else was the same as Example 2. LC MS: M/Z 968.39 (M+). Total yield of synthesis: 45%; HPLC purity: 99.9%.

### Example 53

### Synthesis of Compound 44

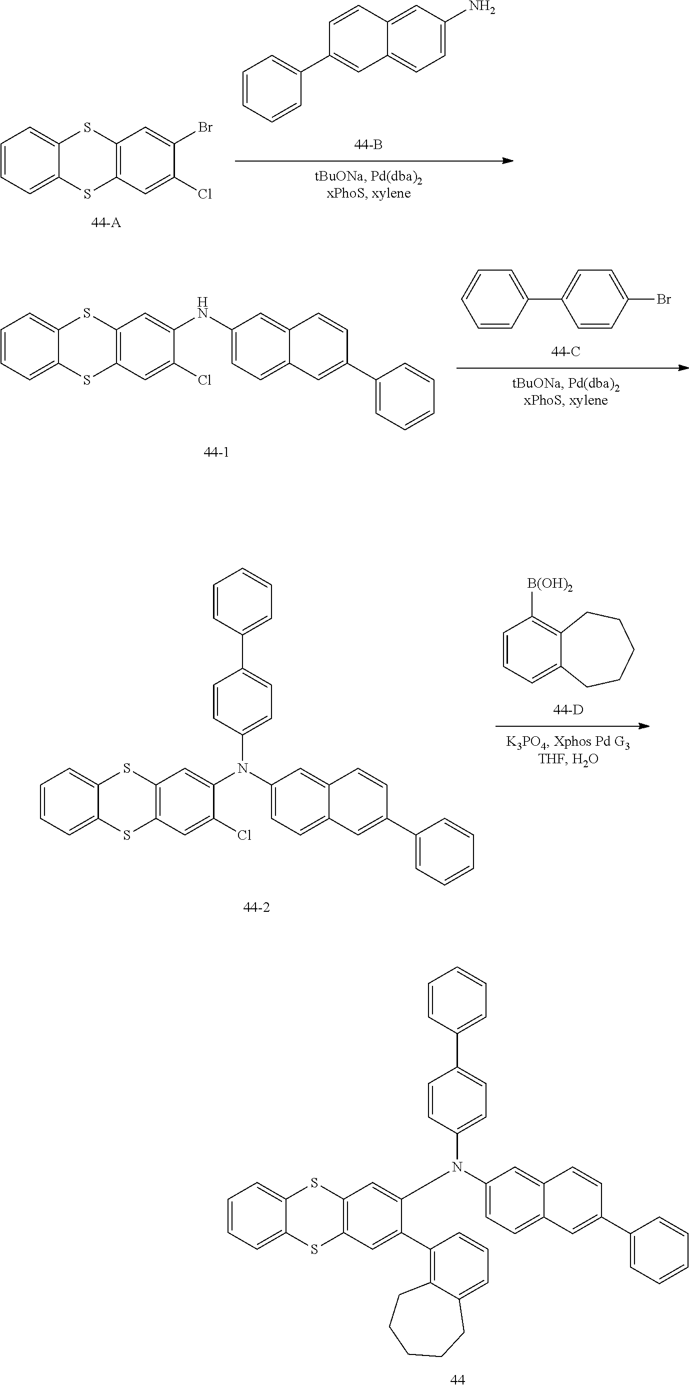

Except that the starting materials were replaced with 44-A, 44-B, 44-C and 44-D, everything else was the same as Example 2. LC MS: M/Z 729.25 (M+). Total yield of synthesis: 46%; HPLC purity: 99.9%.

### Example 54

### Synthesis of Compound 55

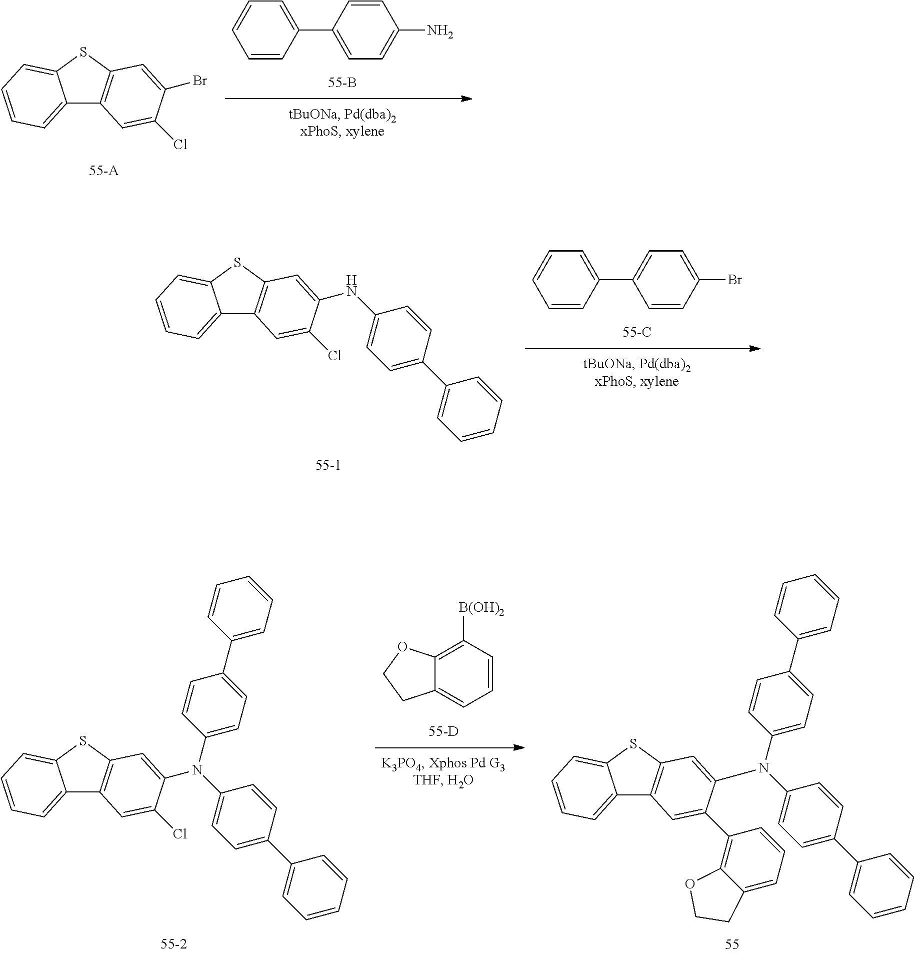

Except that the starting materials were replaced with 55-A, 55-B, 55-C and 55-D, everything else was the same as Example 2. LC MS: M/Z 621.21 (M+). Total yield of synthesis: 47%; HPLC purity: 99.9%.

### Example 55

### Synthesis of Compound 57

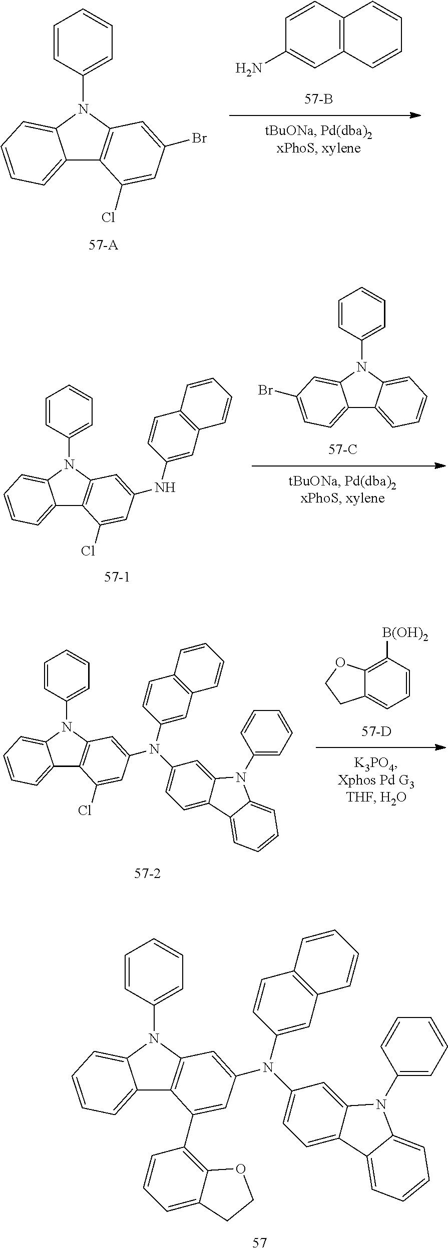

Except that the starting materials were replaced with 57-A, 57-B, 57-C and 57-D, everything else was the same as Example 2. LC MS: M/Z 743.29 (M+). Total yield of synthesis: 46%; HPLC purity: 99.9%.

### Example 56

### Synthesis of Compound 64

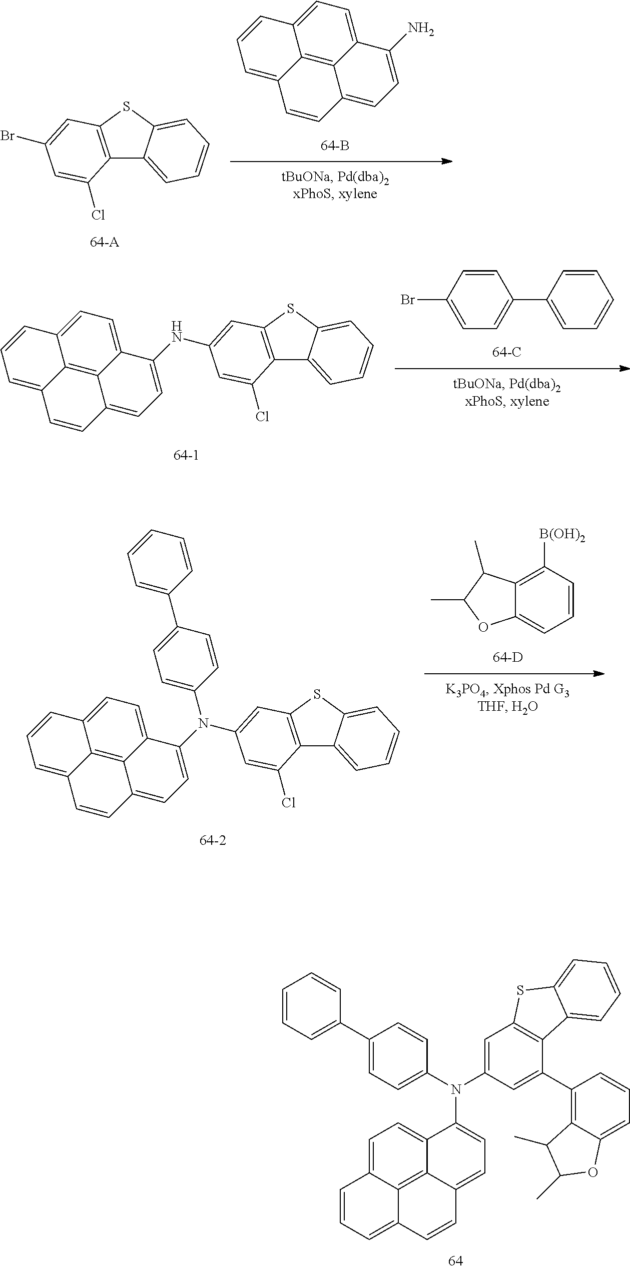

Except that the starting materials were replaced with 64-A, 64-B, 64-C and 64-D, everything else was the same as Example 2. LC MS: M/Z 697.24 (M+). Total yield of synthesis: 47%; HPLC purity: 99.9%.

### Example 57

### Synthesis of Compound 68

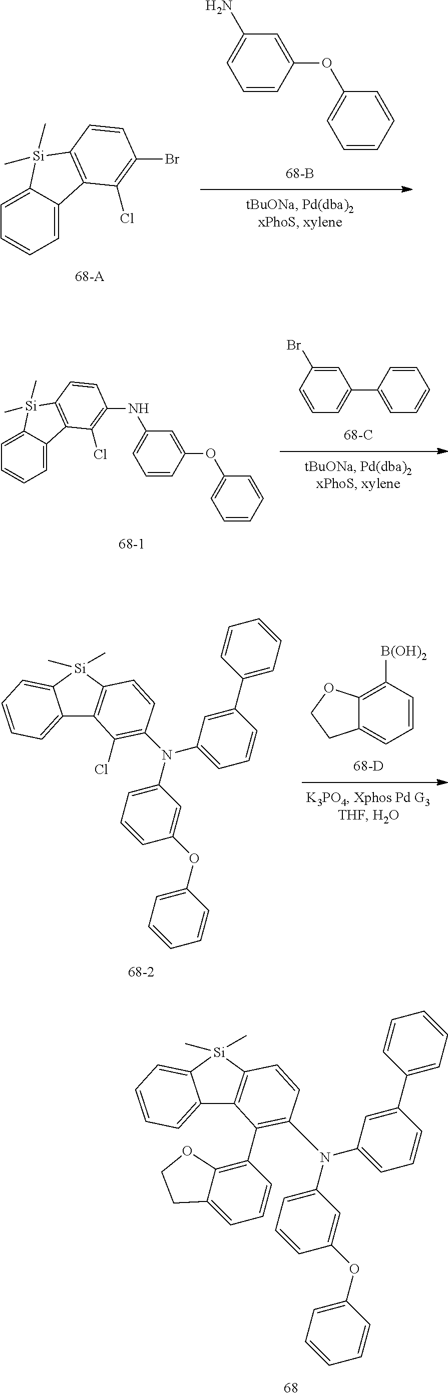

Except that the starting materials were replaced with 68-A, 68-B, 68-C and 68-D, everything else was the same as Example 2. LC MS: M/Z 663.26 (M+). Total yield of synthesis: 47%; HPLC purity: 99.9%.

### Example 58

### Synthesis of Compound 93

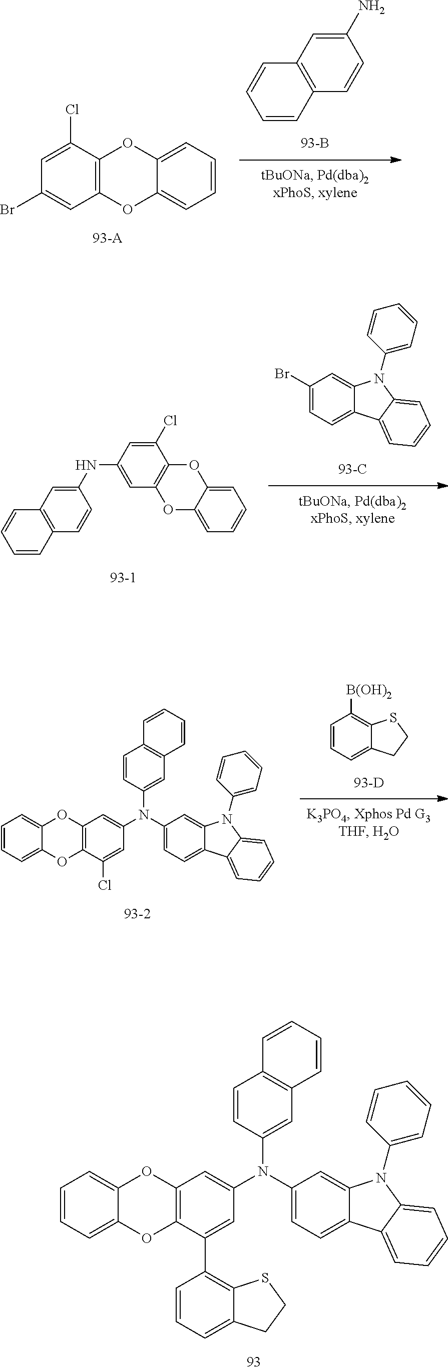

Except that the starting materials were replaced with 93-A, 93-B, 93-C and 93-D, everything else was the same as Example 2. LC MS: M/Z 700.20 (M+). Total yield of synthesis: 48%; HPLC purity: 99.9%.

### Example 59

### Synthesis of Compound 98

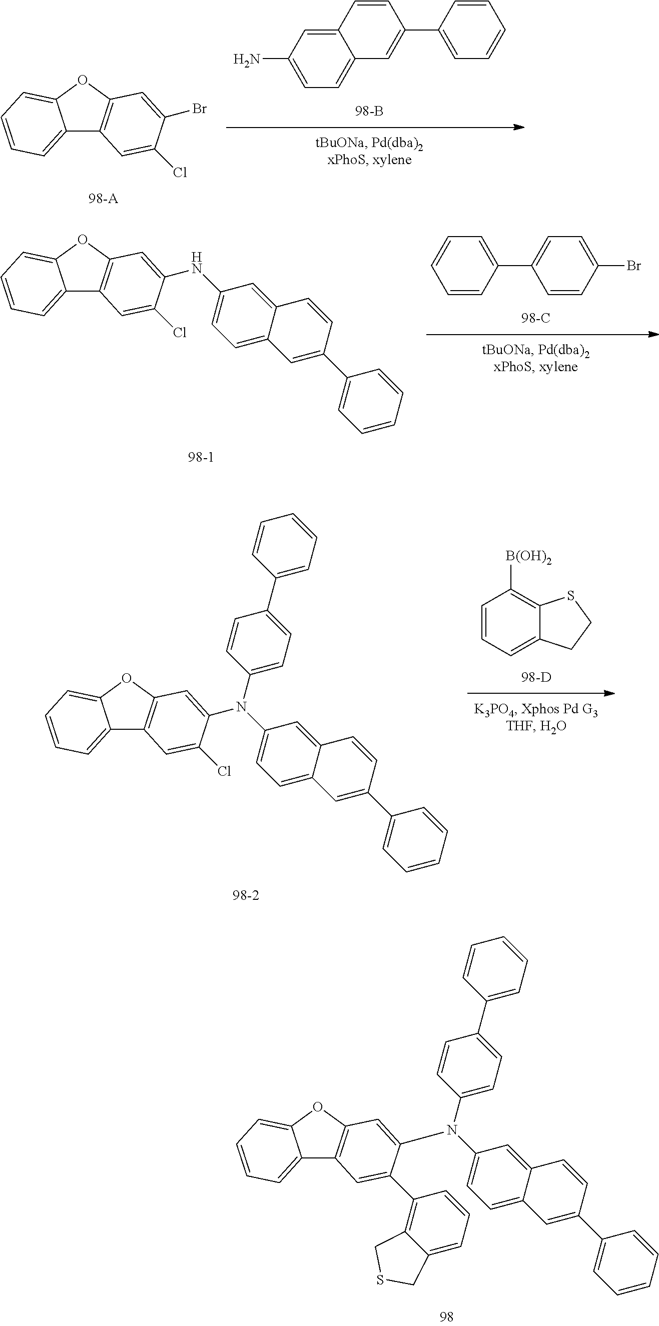

Except that the starting materials were replaced with 98-A, 98-B, 98-C and 98-D, everything else was the same as Example 2. LC MS: M/Z 671.23 (M+). Total yield of synthesis: 46%; HPLC purity: 99.9%.

### Example 60

### Synthesis of Compound 100

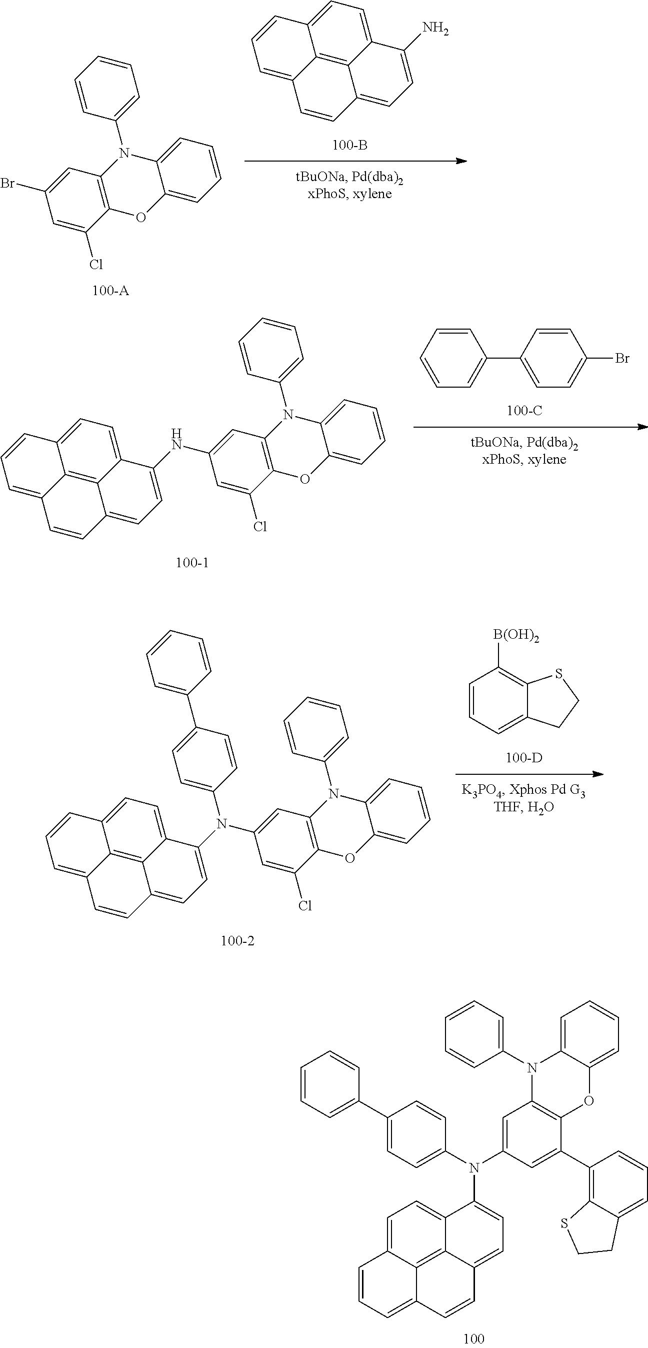

Except that the starting materials were replaced with 100-A, 100-B, 100-C and 100-D, everything else was the same as Example 2. LC MS: M/Z 760.25 (M+). Total yield of synthesis: 46%; HPLC purity: 99.9%.

### Example 61

### Synthesis of Compound 127

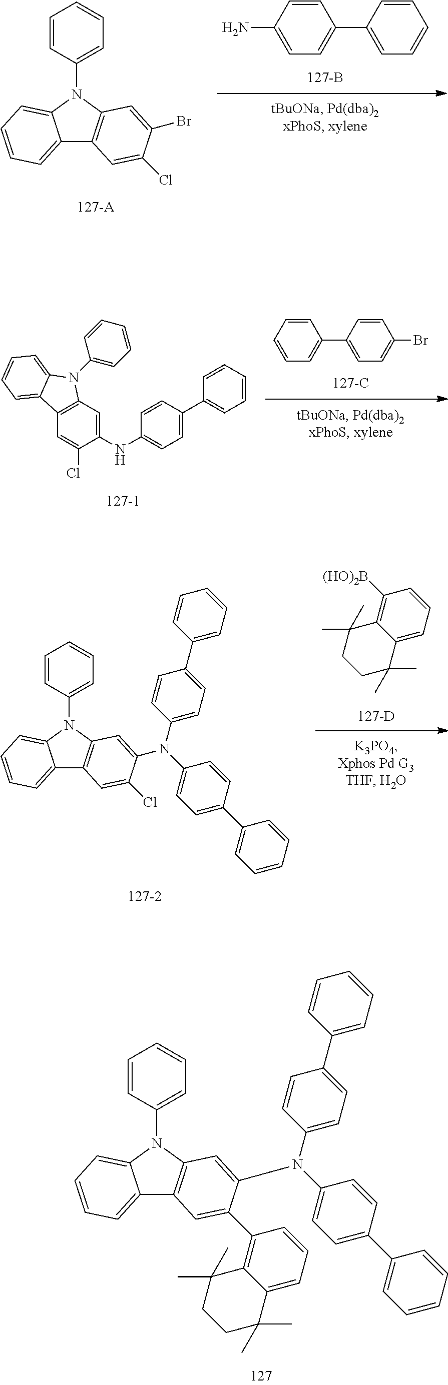

Except that the starting materials were replaced with 127-A, 127-B, 127-C and 127-D, everything else was the same as Example 2. LC MS: M/Z 748.38 (M+). Total yield of synthesis: 46%; HPLC purity: 99.9%.

### Example 62

### Synthesis of Compound 145

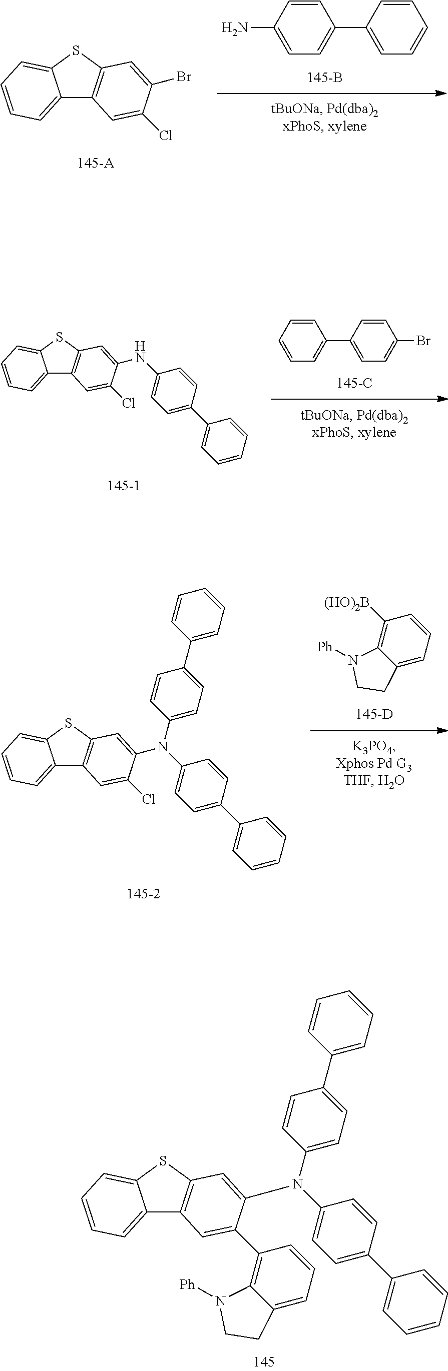

Except that the starting materials were replaced with 145-A, 145-B, 145-C and 145-D, everything else was the same as Example 2. LC MS: M/Z 696.26 (M+). Total yield of synthesis: 49%; HPLC purity: 99.9%.

### Example 63

### Synthesis of Compound 160

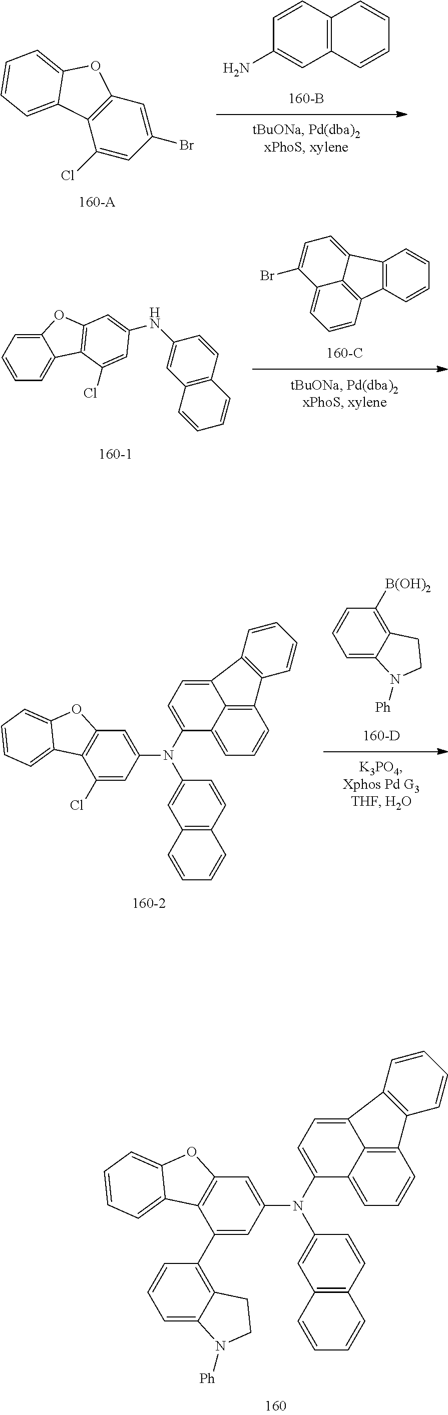

Except that the starting materials were replaced with 160-A, 160-B, 160-C and 160-D, everything else was the same as Example 2. LC MS: M/Z 702.27 (M+). Total yield of synthesis: 47%; HPLC purity: 99.9%.

### Example 64

### Synthesis of Compound 161

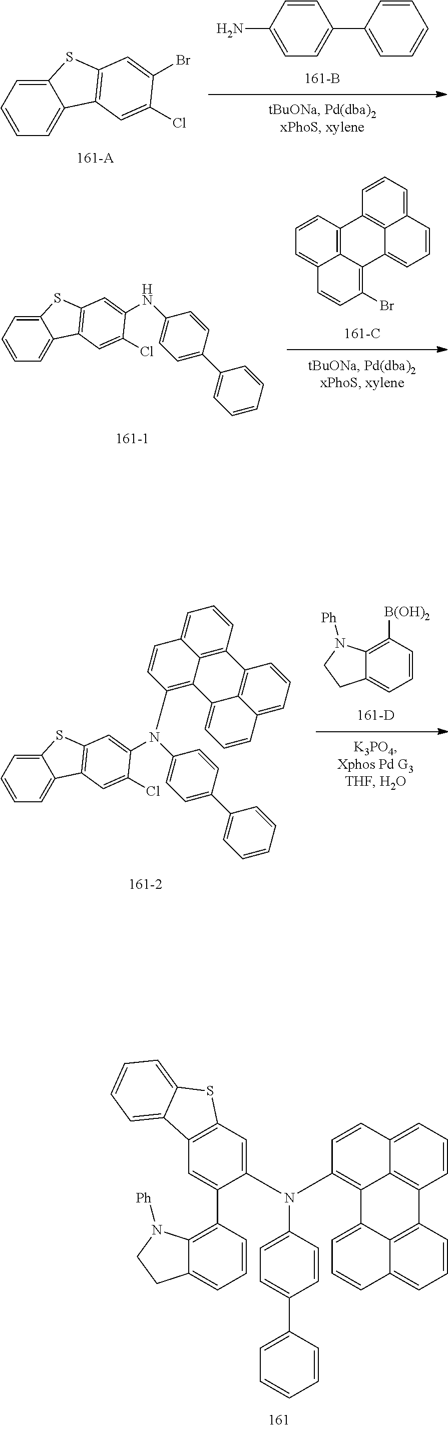

Except that the starting materials were replaced with 161-A, 161-B, 161-C and 161-D, everything else was the same as Example 2. LC MS: M/Z 794.28 (M+). Total yield of synthesis: 47%; HPLC purity: 99.9%.

### Example 65

### Synthesis of Compound 170

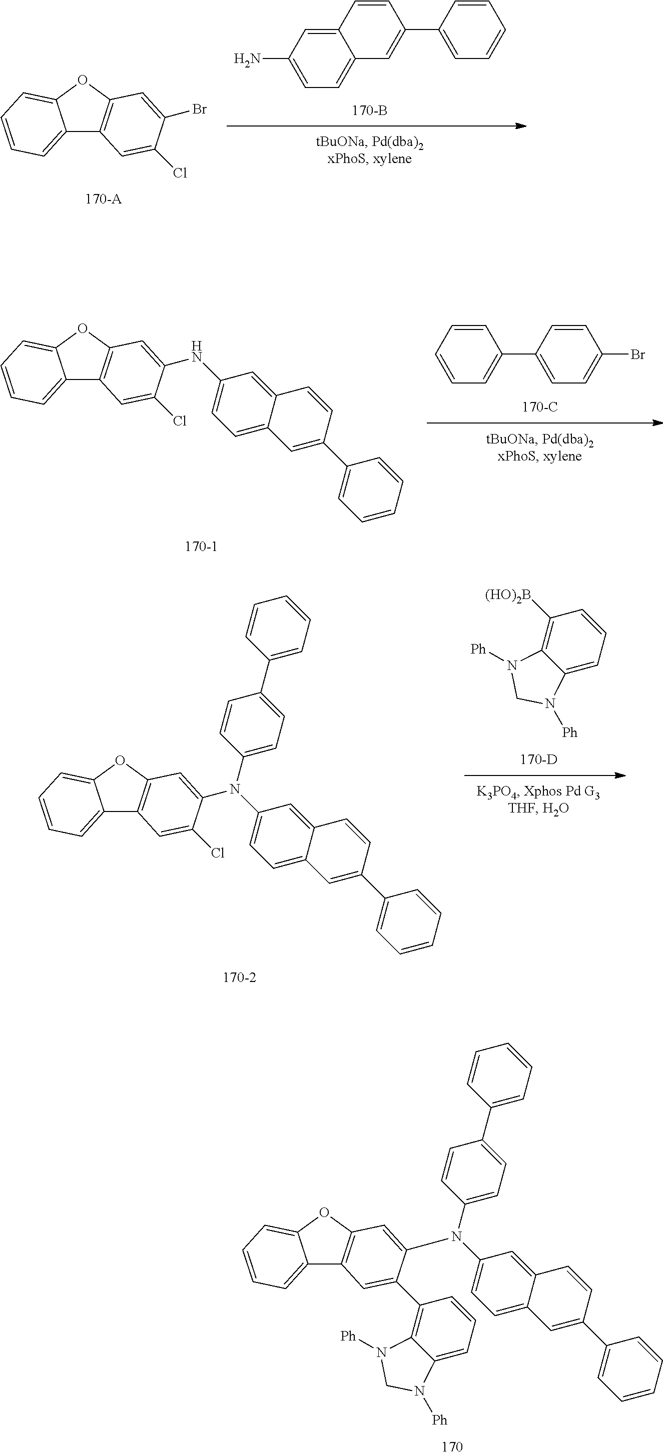

Except that the starting materials were replaced with 170-A, 170-B, 170-C and 170-D, everything else was the same as Example 2. LC MS: M/Z 807.32 (M+). Total yield of synthesis: 47%; HPLC purity: 99.9%.

### Example 66

### Synthesis of Compound 175

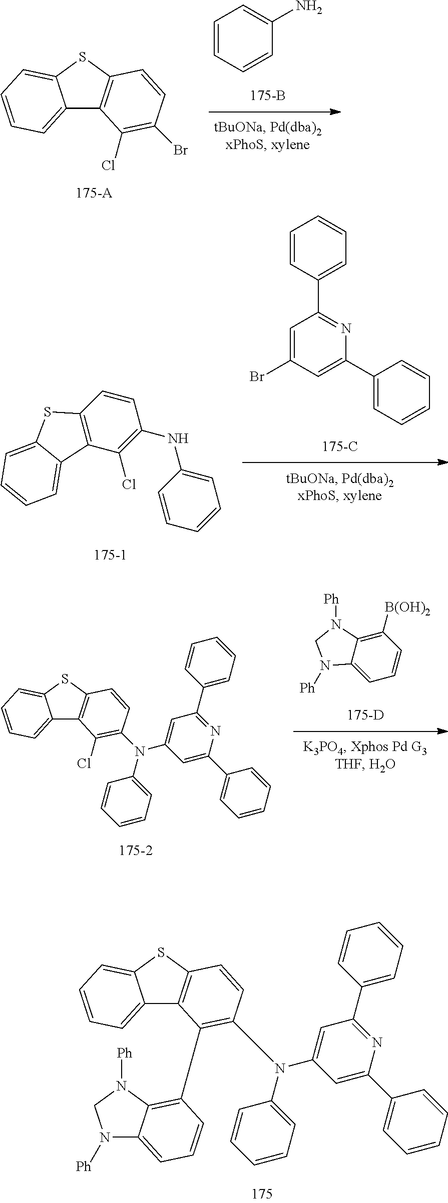

Except that the starting materials were replaced with 175-A, 175-B, 175-C and 175-D, everything else was the same as Example 2. LC MS: M/Z 774.28 (M+). Total yield of synthesis: 46%; HPLC purity: 99.9%.

### Example 67

### Synthesis of Compound 183

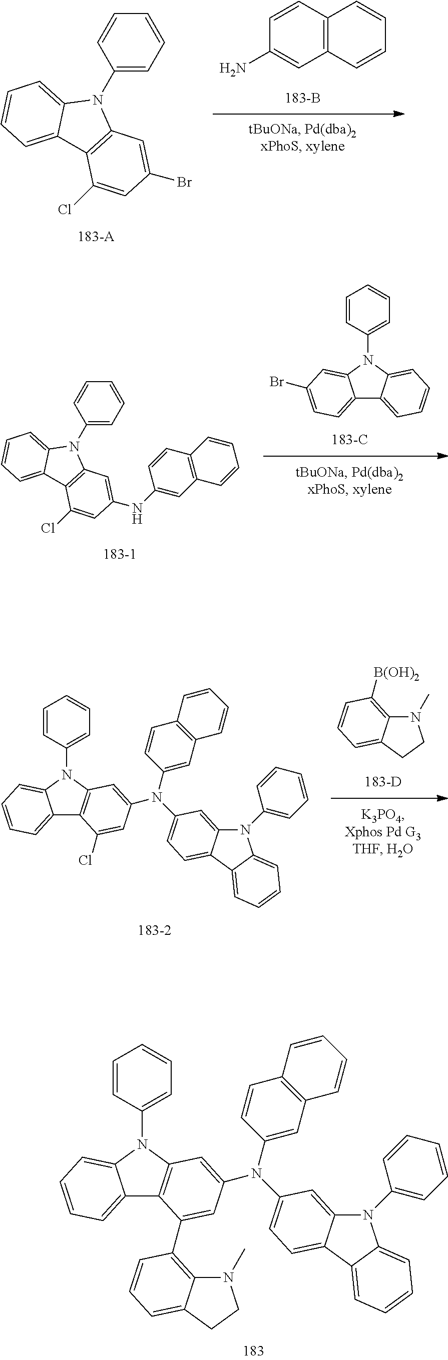

Except that the starting materials were replaced with 183-A, 183-B, 183-C and 183-D, everything else was the same as Example 2. LC MS: M/Z 756.33 (M+). Total yield of synthesis: 47%; HPLC purity: 99.9%.

### Example 68

### Synthesis of Compound 204

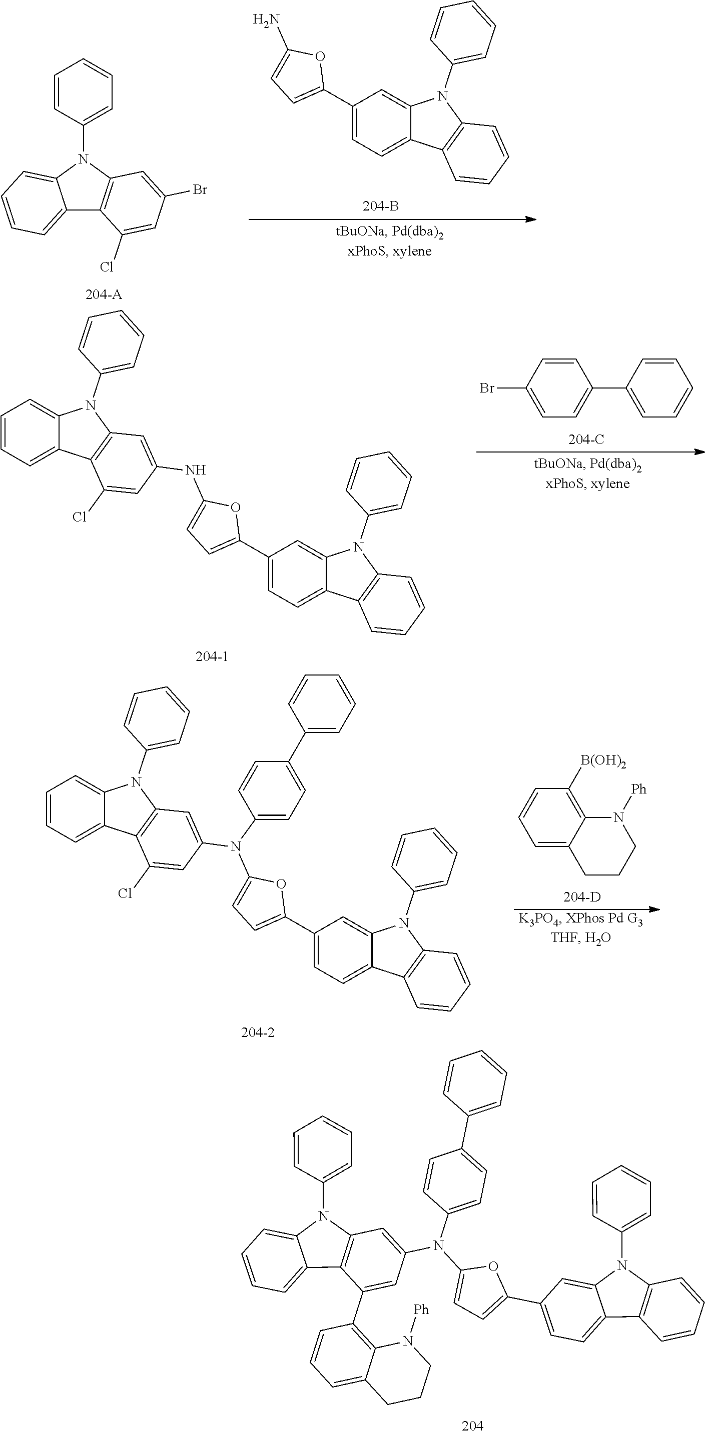

Except that the starting materials were replaced with 204-A, 204-B, 204-C and 204-D, everything else was the same as Example 2. LC MS: M/Z 924.38 (M+). Total yield of synthesis: 47%; HPLC purity: 99.9%.

### Example 69

### Synthesis of Compound 220

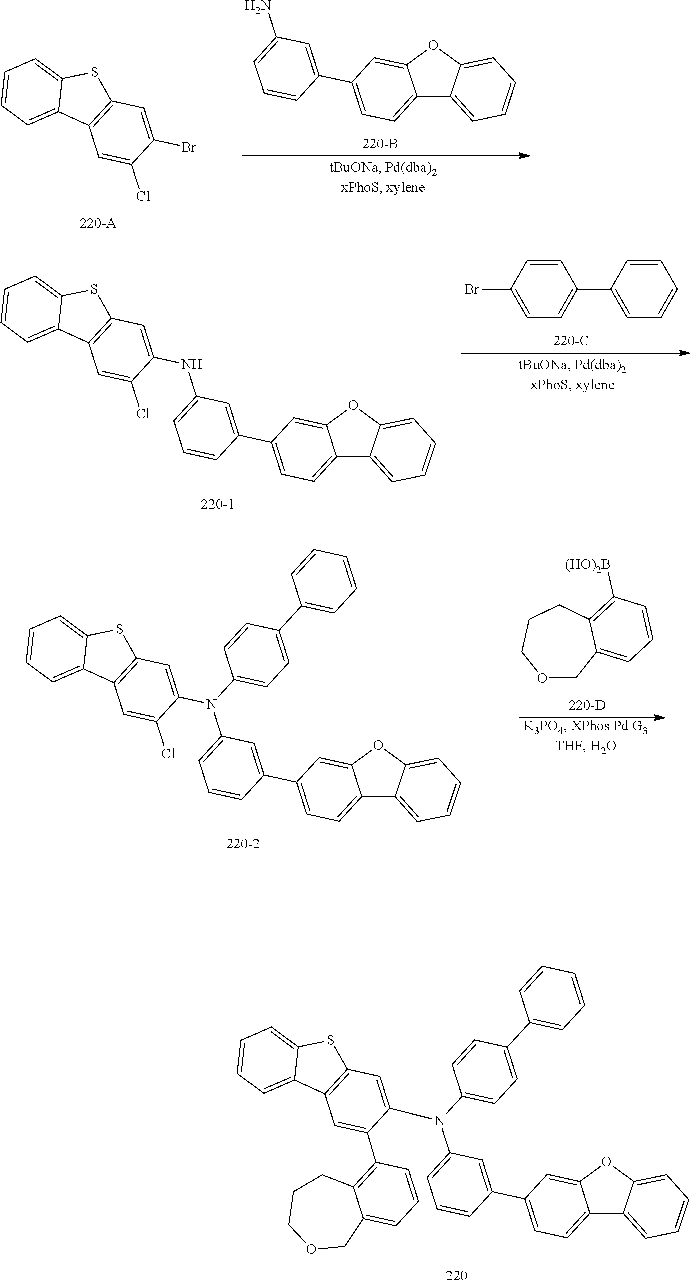

Except that the starting materials were replaced with 220-A, 220-B, 220-C and 220-D, everything else was the same as Example 2. LC MS: M/Z 739.25 (M+). Total yield of synthesis: 47%; HPLC purity: 99.9%.

### Example 70

### Synthesis of Compound 256

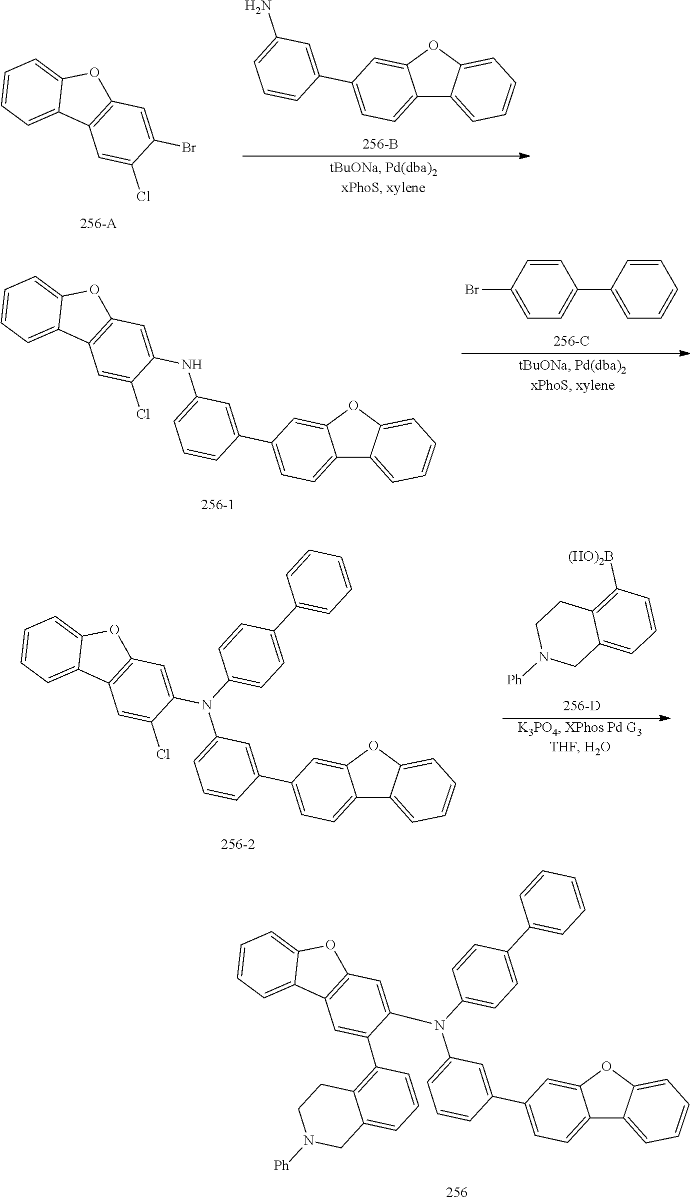

Except that the starting materials were replaced with 256-A, 256-B, 256-C and 256-D, everything else was the same as Example 2. LC MS: M/Z 784.31 (M+). Total yield of synthesis: 48%; HPLC purity: 99.9%.

### Example 71

### Synthesis of Compound 293

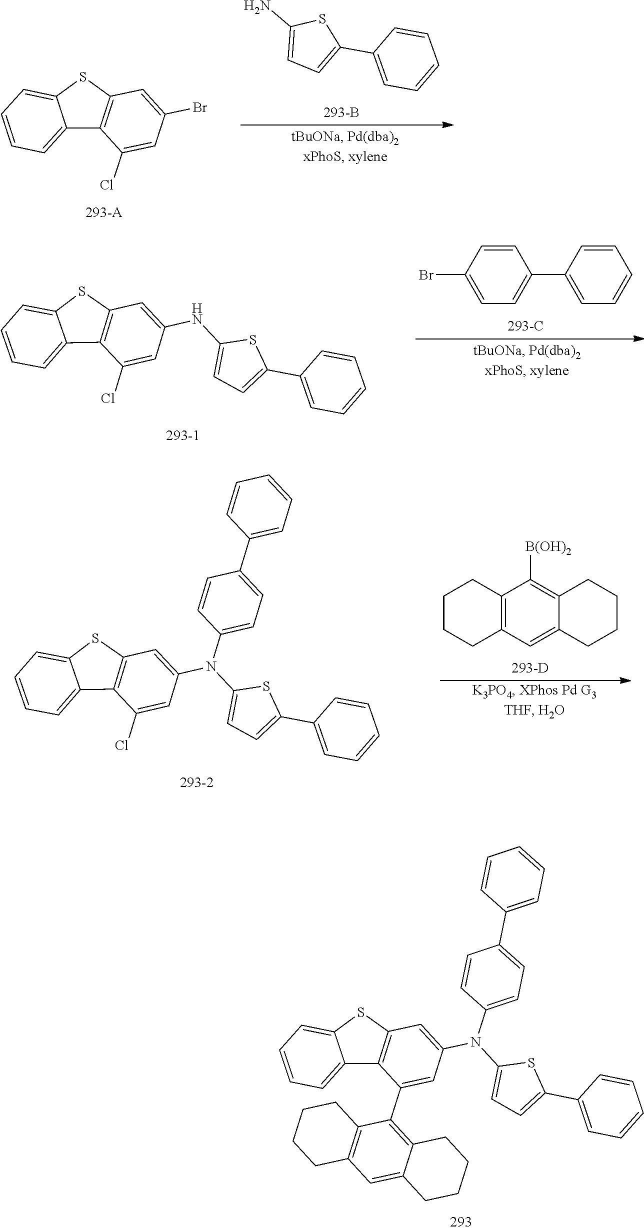

Except that the starting materials were replaced with 293-A, 293-B, 293-C and 293-D, everything else was the same as Example 2. LC MS: M/Z 809.45 (M+). Total yield of synthesis: 48%; HPLC purity: 99.9%.

### Example 72

### Synthesis of Compound 305

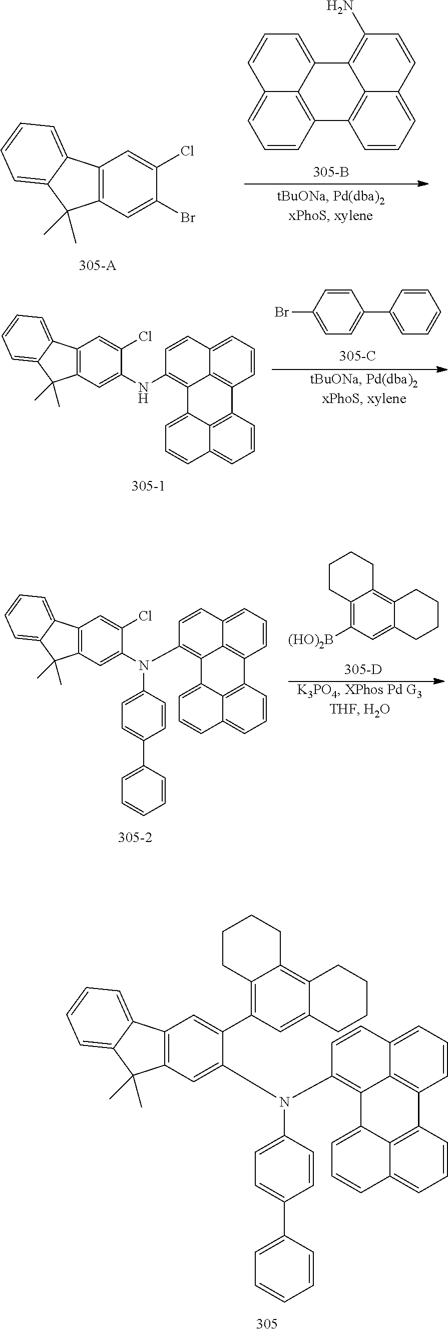

Except that the starting materials were replaced with 305-A, 305-B, 305-C and 305-D, everything else was the same as Example 2. LC MS: M/Z 795.39 (M+). Total yield of synthesis: 46%; HPLC purity: 99.9%.

### Example 73

### Synthesis of Compound 307

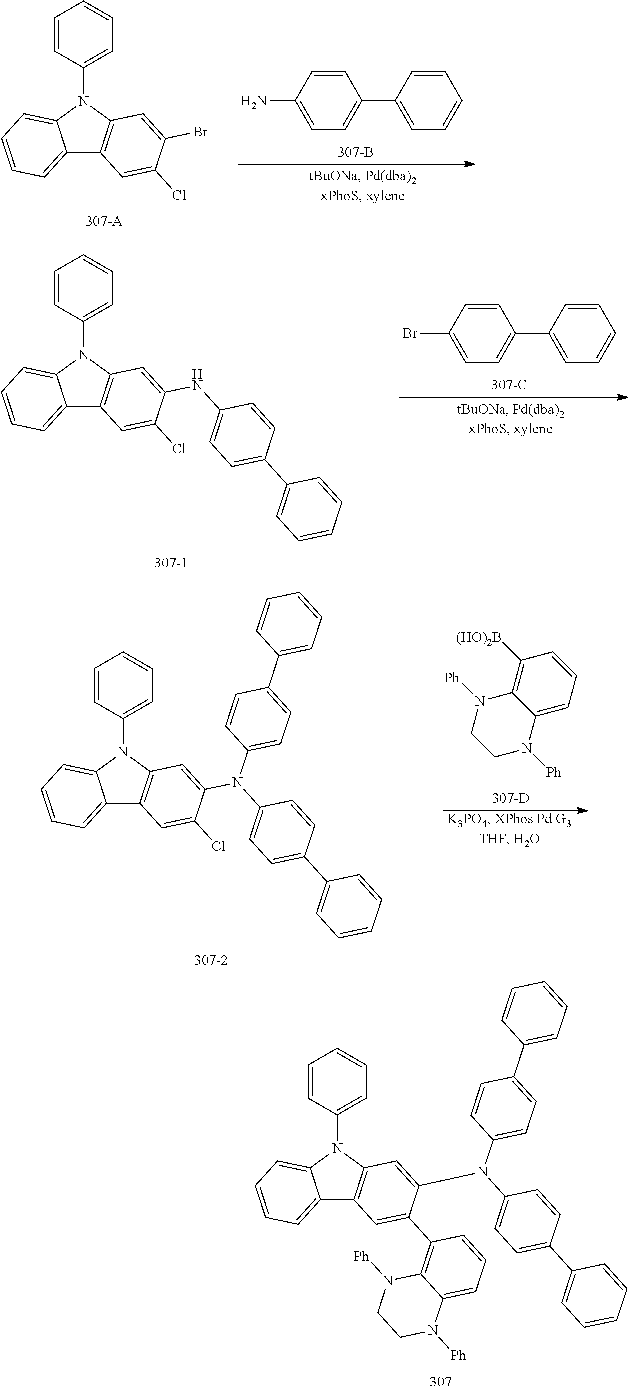

Except that the starting materials were replaced with 307-A, 307-B, 307-C and 307-D, everything else was the same as Example 2. LC MS: M/Z 846.37 (M+). Total yield of synthesis: 46%; HPLC purity: 99.9%.

### Example 74

### Synthesis of Compound 352

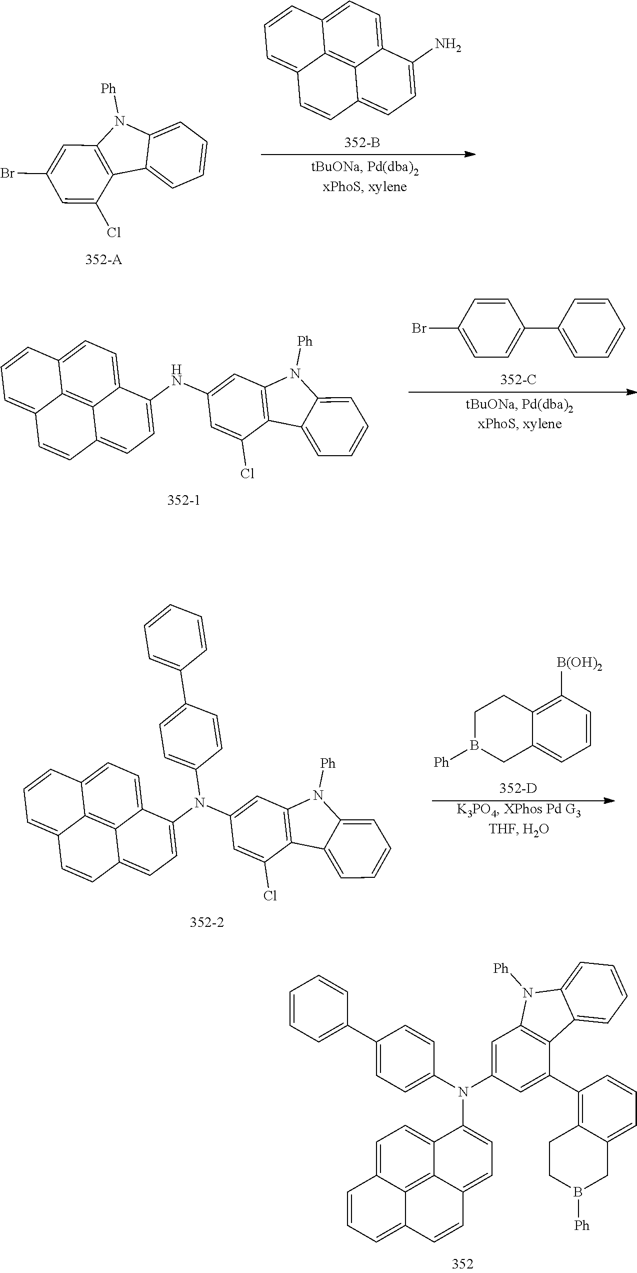

Except that the starting materials were replaced with 352-A, 352-B, 352-C and 352-D, everything else was the same as Example 2. LC MS: M/Z 814.35 (M+). Total yield of synthesis: 48%; HPLC purity: 99.9%.

### Example 75

### Synthesis of Compound 355

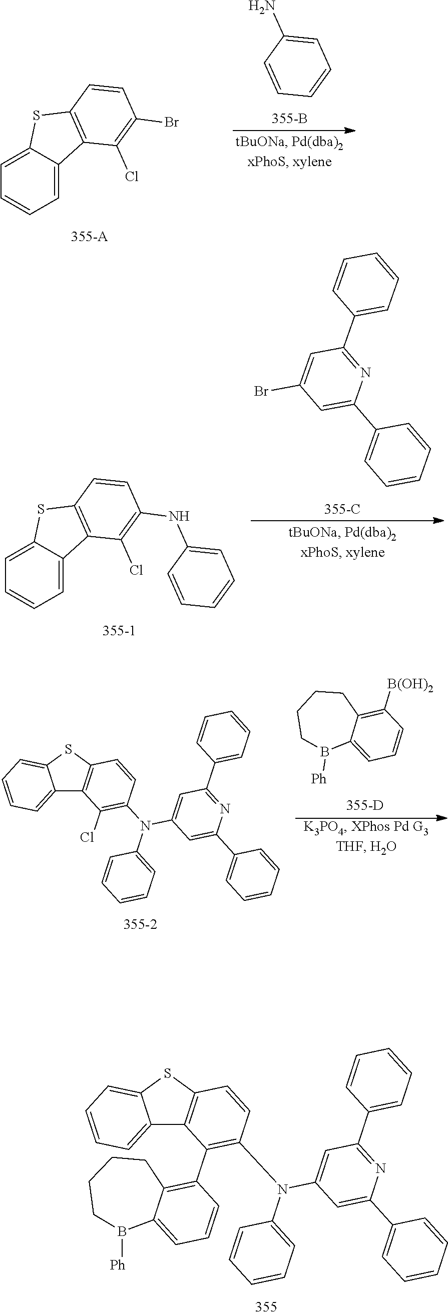

Except that the starting materials were replaced with 355-A, 355-B, 355-C and 355-D, everything else was the same as Example 2. LC MS: M/Z 722.29 (M+). Total yield of synthesis: 49%; HPLC purity: 99.9%.

### Example 76

### Synthesis of Compound 363

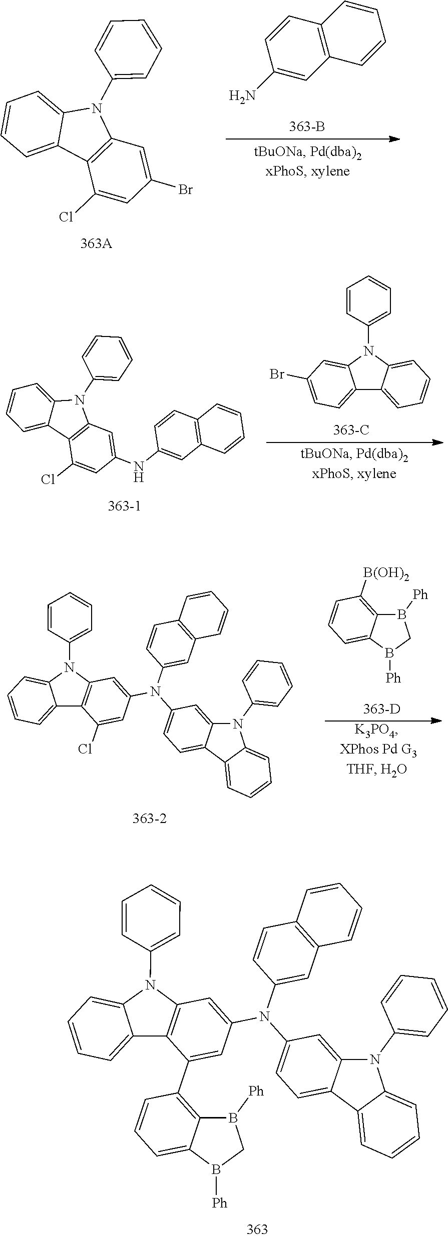

Except that the starting materials were replaced with 363-A, 363-B, 363-C and 363-D, everything else was the same as Example 2. LC MS: M/Z 889.38 (M+). Total yield of synthesis: 46%; HPLC purity: 99.9%.

### Example 77

### Synthesis of Compound 391

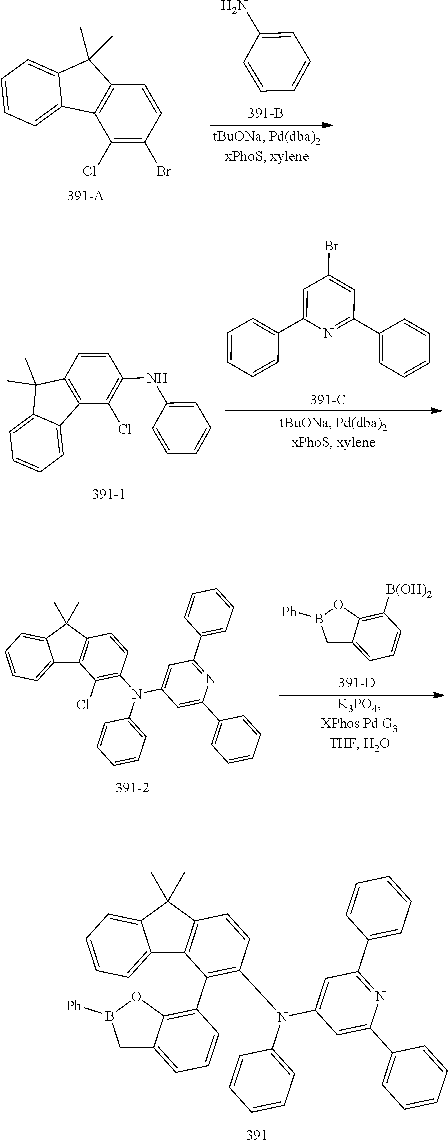

Except that the starting materials were replaced with 391-A, 391-B, 391-C and 391-D, everything else was the same as Example 2. LC MS: M/Z 706.32 (M+). Total yield of synthesis: 49%; HPLC purity: 99.9%.

### Example 78

### Synthesis of Compound 403

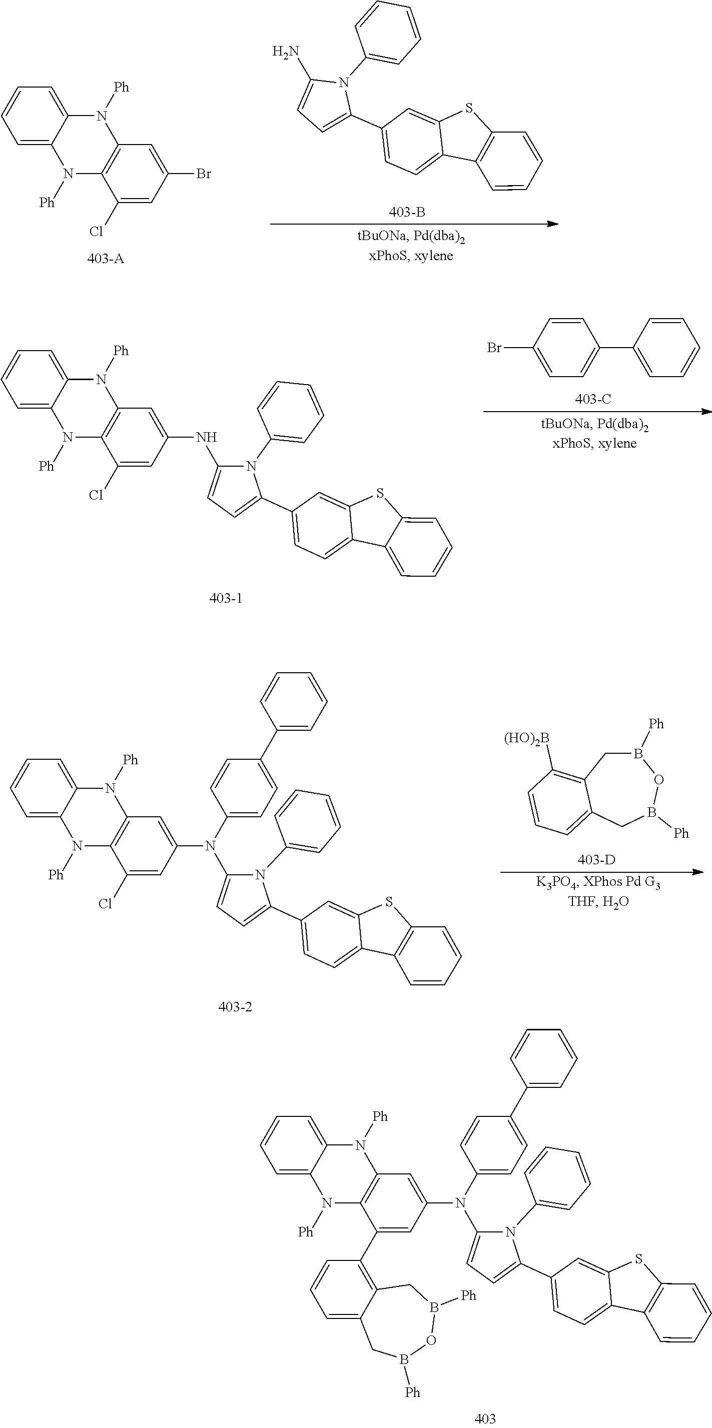

Except that the starting materials were replaced with 403-A, 403-B, 403-C and 403-D, everything else was the same as Example 2. LC MS: M/Z 1118.44 (M+). Total yield of synthesis: 48%; HPLC purity: 99.9%.

### Example 79

### Synthesis of Compound 417

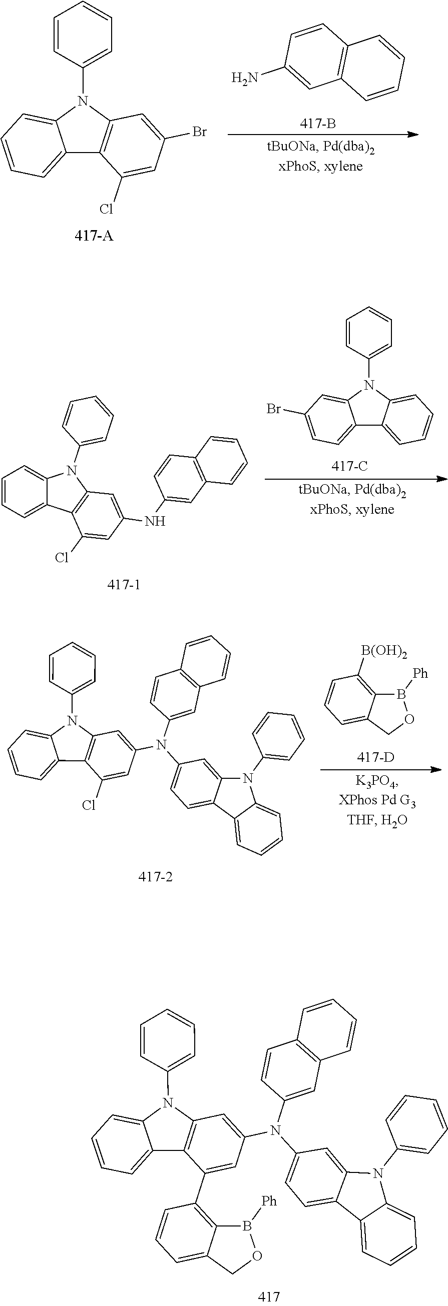

Except that the starting materials were replaced with 417-A, 417-B, 417-C and 417-D, everything else was the same as Example 2. LC MS: M/Z 817.33 (M+). Total yield of synthesis: 48%; HPLC purity: 99.9%.

### Example 80

### Synthesis of Compound 428

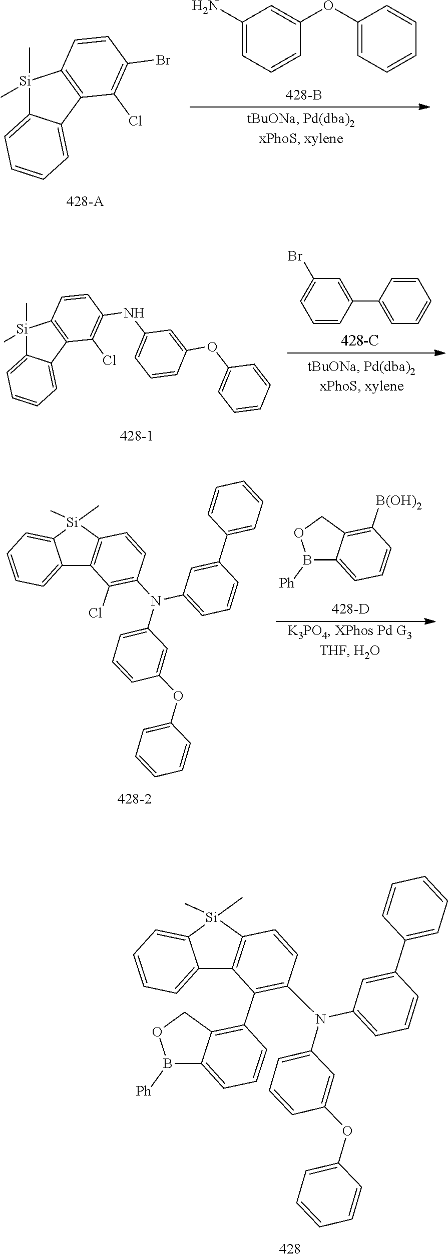

Except that the starting materials were replaced with 428-A, 428-B, 428-C and 428-D, everything else was the same as Example 2. LC MS: M/Z 737.29 (M+). Total yield of synthesis: 48%; HPLC purity: 99.9%.

### Example 81

### Synthesis of Compound 415

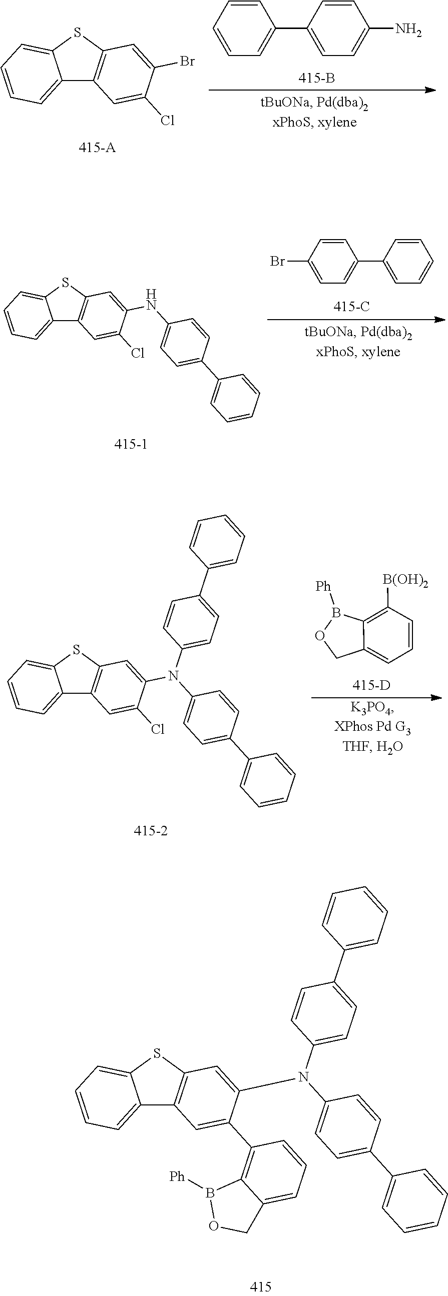

Except that the starting materials were replaced with 415-A, 415-B, 415-C and 415-D, everything else was the same as Example 2. LC MS: M/Z 695.25 (M+). Total yield of synthesis: 48%; HPLC purity: 99.9%.

### Example 82

### Synthesis of Compound 231

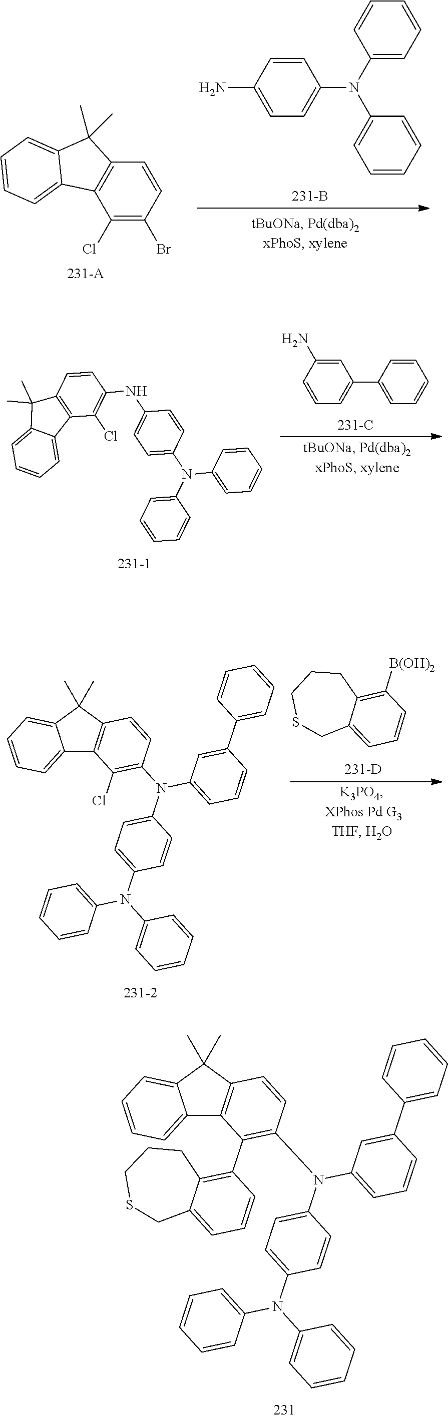

Except that the starting materials were replaced with 231-A, 231-B, 231-C and 231-D, everything else was the same as Example 2. LC MS: M/Z 766.34 (M+). Total yield of synthesis: 47%; HPLC purity: 99.9%.

### Example 83

### Synthesis of Compound 4

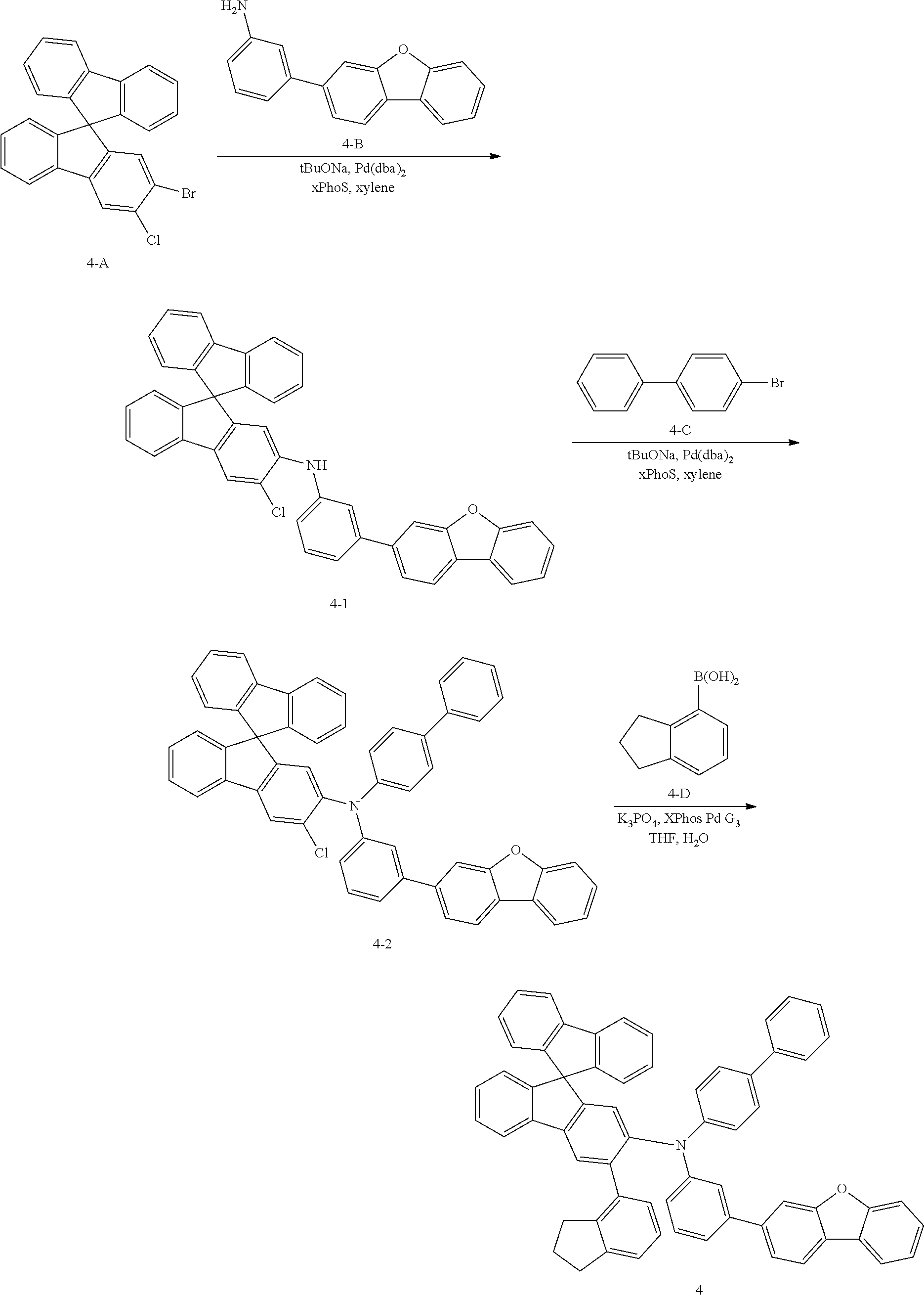

Except that the starting materials were replaced with 4-A, 4-B, 4-C and 4-D, everything else was the same as Example 2. LC MS: M/Z 841.33 (M+). Total yield of synthesis: 47%; HPLC purity: 99.9%.

### Example 84

### Synthesis of Compound 30

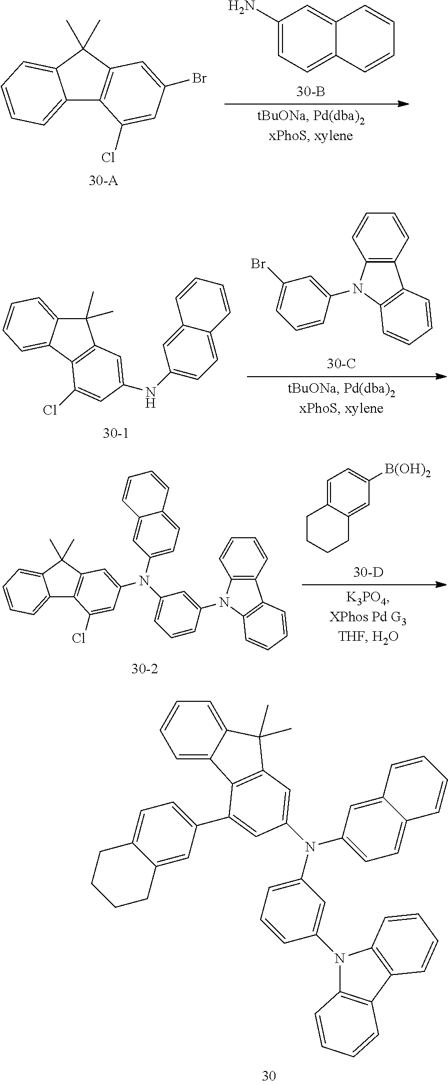

Except that the starting materials were replaced with 30-A, 30-B, 30-C and 30-D, everything else was the same as Example 2. LC MS: M/Z 706.33 (M+). Total yield of synthesis: 46%; HPLC purity: 99.9%.

### Example 85

### Synthesis of Compound 38

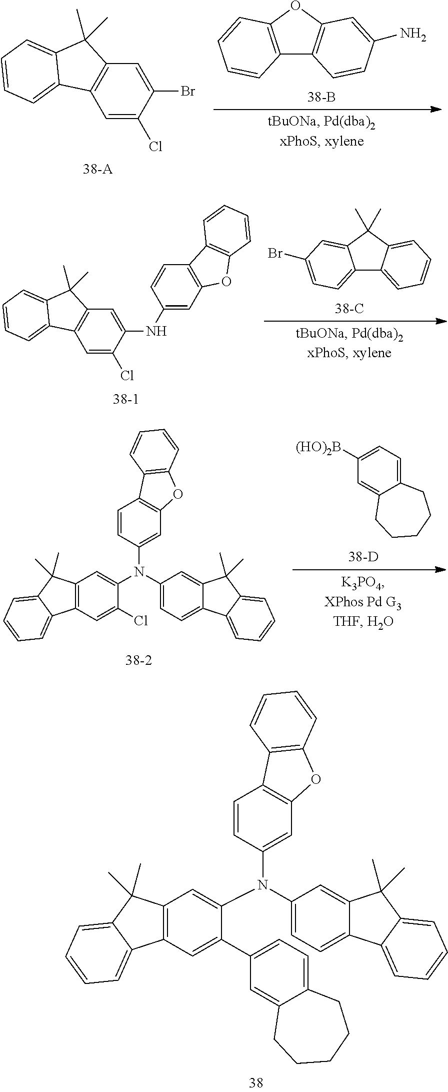

Except that the starting materials were replaced with 38-A, 38-B, 38-C and 38-D, everything else was the same as Example 2. LC MS: M/Z 711.35 (M+). Total yield of synthesis: 49%; HPLC purity: 99.9%.

### Example 86

### Synthesis of Compound 43

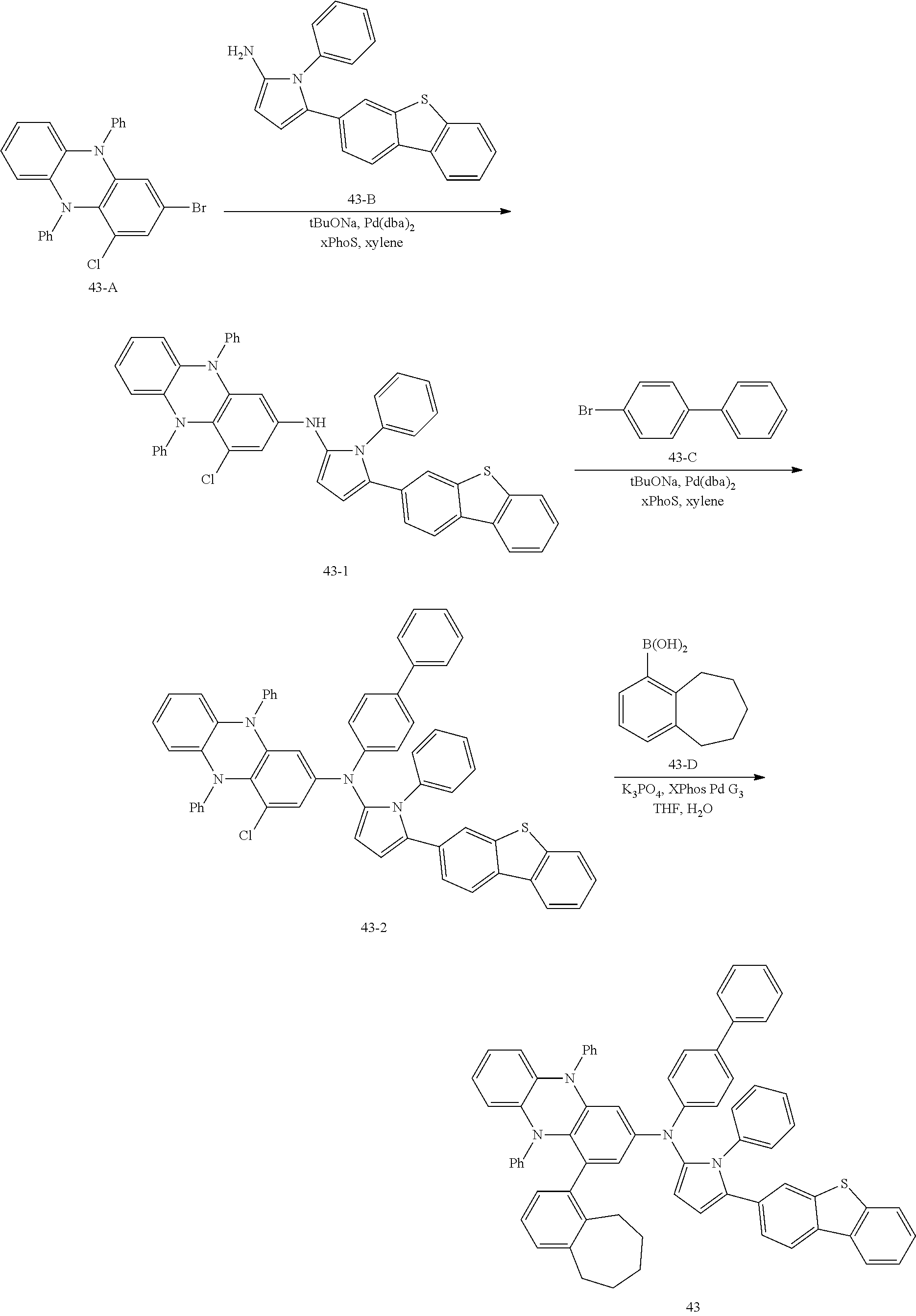

Except that the starting materials were replaced with 43-A, 43-B, 43-C and 43-D, everything else was the same as Example 2. LC MS: M/Z 968.39 (M+). Total yield of synthesis: 47%; HPLC purity: 99.9%.

### Example 87

### Synthesis of Compound 163

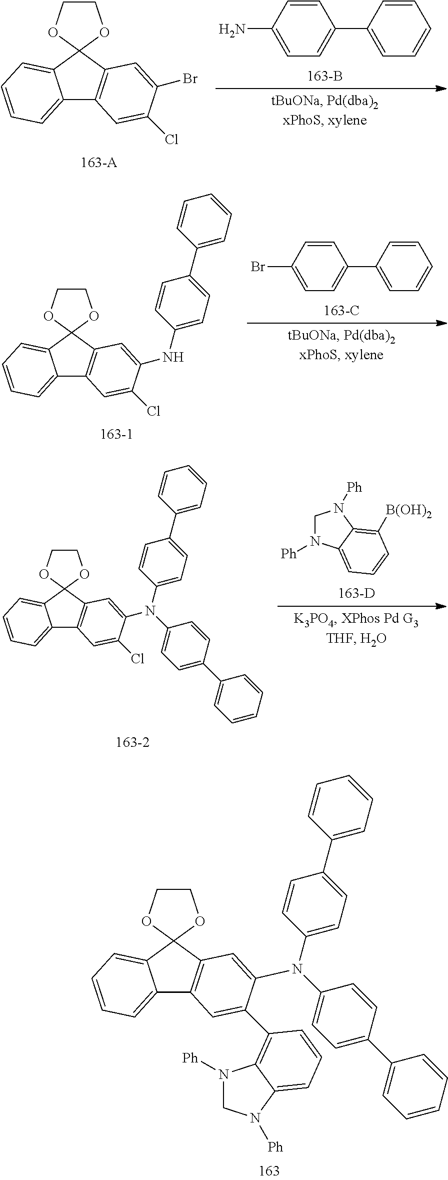

Except that the starting materials were replaced with 163-A, 163-B, 163-C and 163-D, everything else was the same as Example 2. LC MS: M/Z 706.33 (M+). Total yield of synthesis: 48%; HPLC purity: 99.9%.

### Example 88

### Synthesis of Compound 478

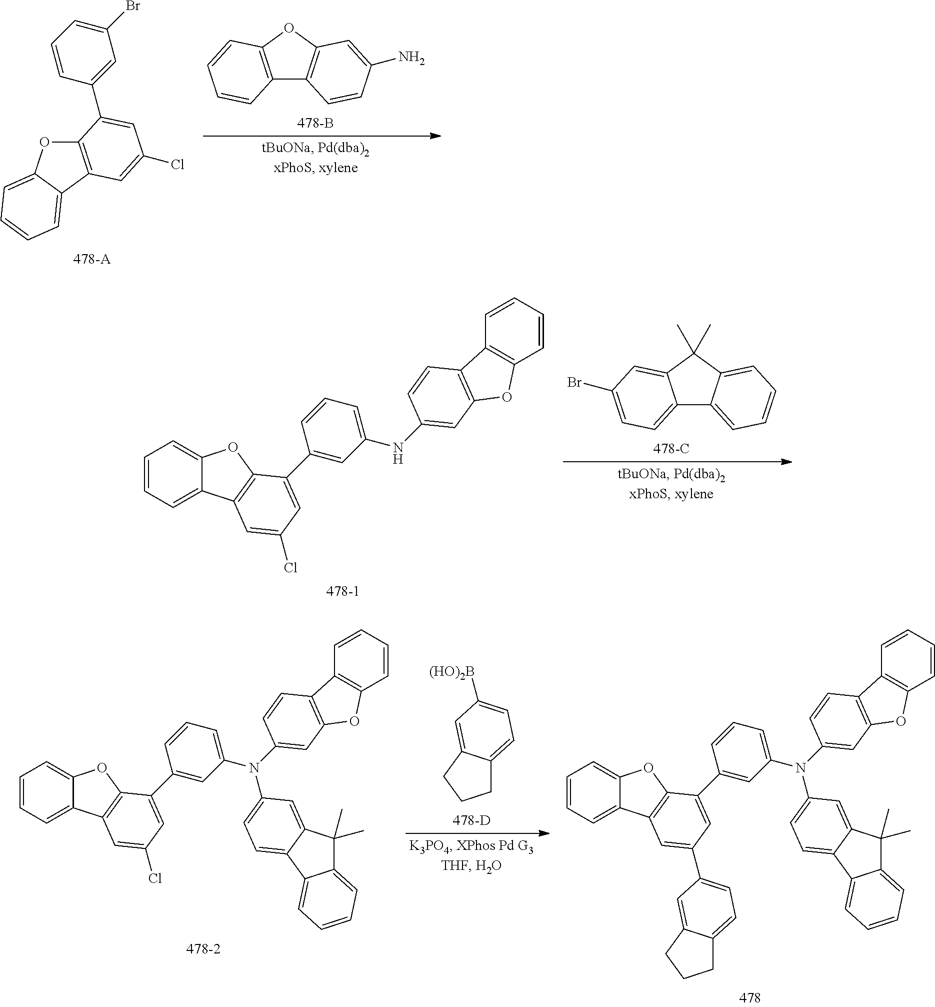

### 1) Synthesis of Intermediate 478-1

Under an argon atmosphere, 35.8 g (100 mmol) of compound 478-A, 18.3 g (100 mmol) of compound 478-B, 23.4 g (240 mmol) of sodium tert-butoxide, 575 mg (1 mmol %) of bis(dibenzylideneacetone)palladium, 953 mg (2 mmol %) of 2-dicyclohexylphosphine-2′,4′,6′-triisopropylbiphenyl and 1000 mL of xylene were added to a reaction container, and heated to 140° C. with stirring for 15 hours. The reaction mixture was cooled to room temperature, and 1000 ml of water was added thereto. The mixture was filtered. The filter cake was washed with a large amount of water and dried in vacuum. The crude product was purified by silica gel column chromatography (eluent: ethyl acetate/hexane) to obtain 34.9 g of compound 478-1 with a HPLC purity of 99.5% and a yield of 82%. LC MS: M/Z 425.14 (M+).

¹H NMR (400 MHz, DMSO-d6) δ 6.91 (m, 1H), 7.02 (s, 1H), 7.23 (m, 1H), 7.31 (m, 2H), 7.37-7.48 (m, 3H), 7.48-7.57 (m, 2H), 7.53-7.62 (m, 1H), 7.65-7.72 (m, 2H), 7.77 (t, 1H), 7.95-8.01 (m, 2H), 8.01-8.09 (m, 2H), 8.12 (m, 1H).

### 2) Synthesis of Intermediate 478-2

Under an argon atmosphere, 42.5 g (100 mmol) of compound 478-1, 27.3 g (100 mmol) of compound 478-C, 23.4 g (240 mmol) of sodium tert-butoxide, 575 mg (1 mmol %) of bis(dibenzylideneacetone)palladium, 953 mg (2 mmol %) of 2-dicyclohexylphosphine-2′,4′,6′-triisopropylbiphenyl and 1000 mL of xylene were added to a reaction container, and heated to 140° C. with stirring for 15 hours. The reaction mixture was cooled to room temperature, and 1000 ml of water was added thereto. The mixture was filtered. The filter cake was washed with a large amount of water and dried in vacuum. The crude product was purified by silica gel column chromatography (eluent: ethyl acetate/hexane) to obtain 51.3 g of compound 478-2 with a HPLC purity of 99.5% and a yield of 83%. LC MS: M/Z 617.24 (M+).

¹H NMR (400 MHz, DMSO-d6) δ 1.69 (s, 6H), 6.91 (m, 1H), 6.95-7.01 (m, 1H), 7.18 (m, 1H), 7.23 (m, 1H), 7.26-7.62 (m, 12H), 7.68 (m, 1H), 7.77 (t, 1H), 7.82-7.91 (m, 2H), 7.95-8.01 (m, 2H), 8.01-8.09 (m, 2H), 8.09-8.15 (m, 1H).

### 3) Synthesis of Compound 478

Under an argon atmosphere, 61.8 g (100 mmol) of compound 478-2, 16.2 g (100 mmol) of compound 478-D, 787 mg (1 mmol %) of XPhos Pd G3, 50 ml (300 mmol) of 1.5 M potassium phosphate and 1000 ml of tetrahydrofuran (THF) were added to a reaction container and stirred under reflux overnight. After cooling to room temperature, 800 ml of water was added thereto, and a large amount of solid precipitated. The solid was filtered, and the filter cake was washed with water three times and dried in vacuum. The crude product was purified by silica gel column chromatography (eluent: ethyl acetate/hexane) to obtain 52.8 g of compound 478 with a yield of 72% and a HPLC purity of 99.9%. LC MS: M/Z 733.30 (M+).

¹H NMR (400 MHz, DMSO-d6) δ 1.69 (s, 6H), 2.07 (m, 2H), 2.77-2.99 (m, 4H), 6.91 (m, 1H), 6.95-7.01 (m, 1H), 7.20 (m, 2H), 7.26-7.63 (m, 14H), 7.68 (m, 1H), 7.77 (t, 1H), 7.81-7.91 (m, 3H), 7.93 (d, 1H), 7.95-8.01 (m, 2H), 8.03 (m, 1H).

### Example 89

### Synthesis of Compound 479

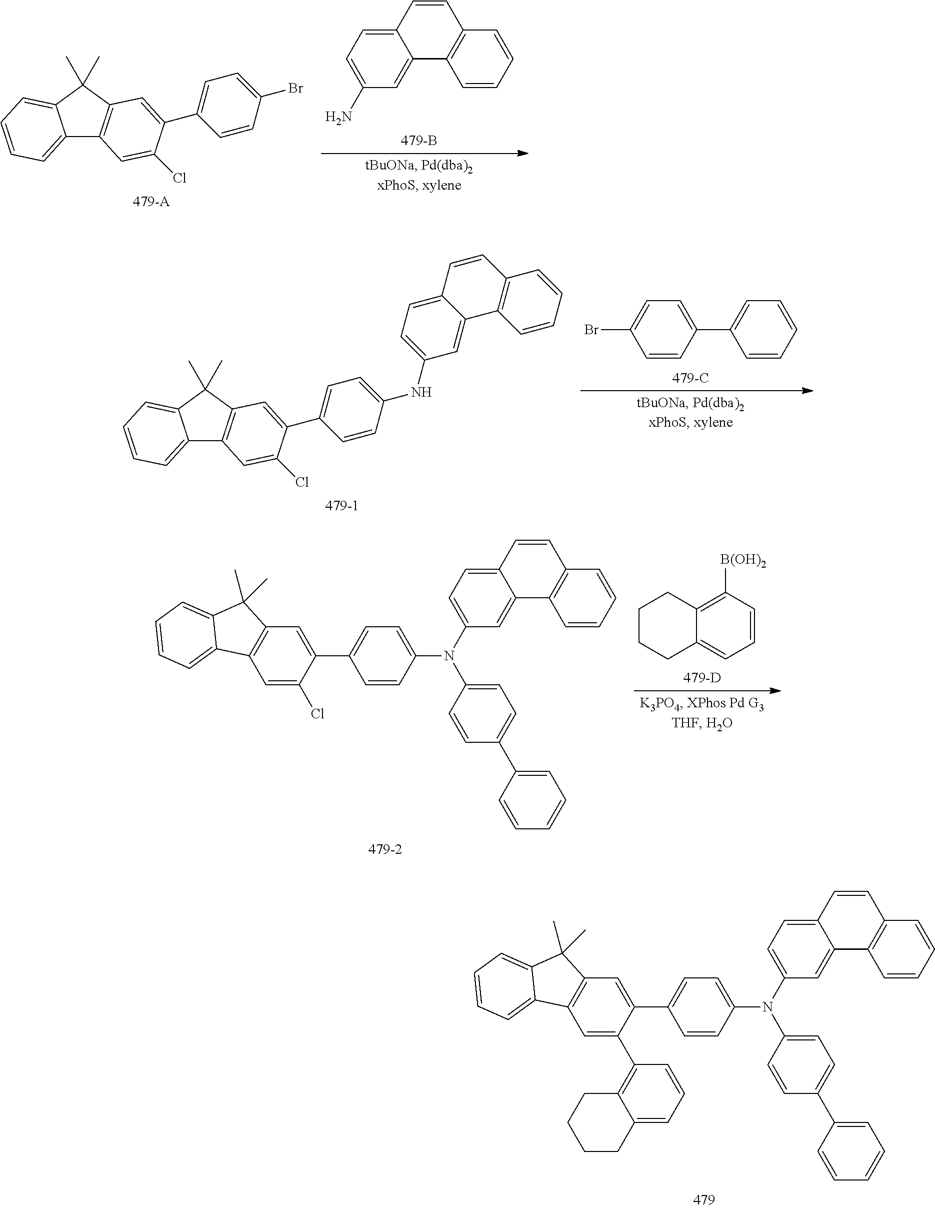

Except that the starting materials were replaced with 479-A, 479-B, 479-C and 479-D, everything else was the same as Example 88. LC MS: M/Z 743.36 (M+). Total yield of synthesis: 48%; HPLC purity: 99.9%.

¹H NMR (400 MHz, DMSO-d6) δ 1.40-1.55 (m, 2H), 1.69 (s, 6H), 1.69-1.81 (m, 2H), 2.37-2.45 (m, 2H), 2.65-2.75 (m, 2H), 6.67 (m, 1H), 7.17-7.79 (m, 23H), 7.80-7.94 (m, 3H), 7.99-8.07 (m, 1H), 8.27 (d, 1H), 8.37 (s, 1H), 8.73-8.81 (m, 1H).

### Example 90

### Synthesis of Compound 480

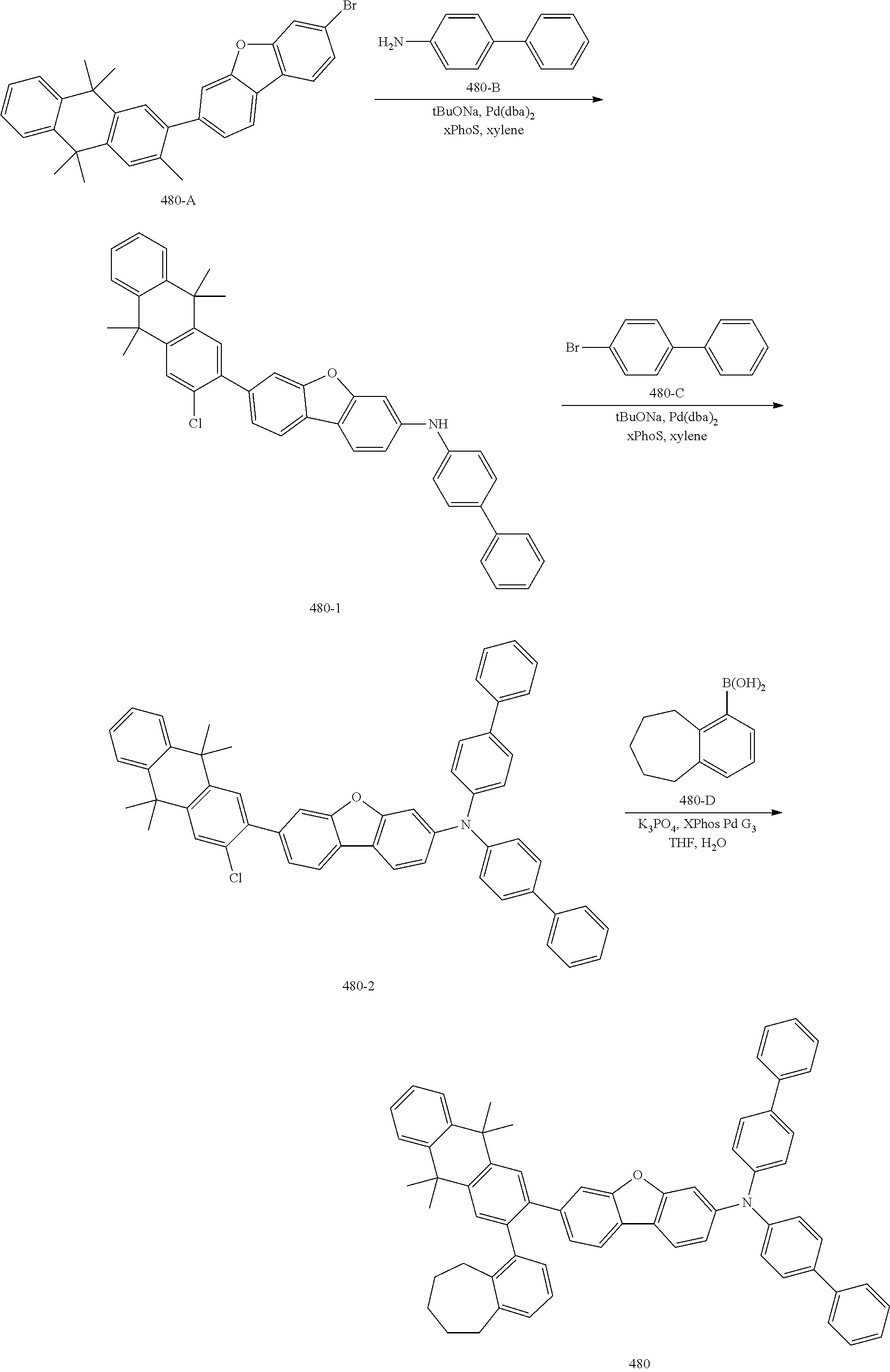

Except that the starting materials were replaced with 480-A, 480-B, 480-C and 480-D, everything else was the same as Example 88. LC MS: M/Z 865.43 (M+). Total yield of synthesis: 47%; HPLC purity: 99.9%.

¹H NMR (400 MHz, DMSO-d6) δ 1.69 (d, 12H), 1.70-1.85 (m, 5H), 1.82-1.91 (m, 1H), 2.78-2.88 (m, 4H), 6.73 (m, 2H), 6.91 (m, 1H), 7.06 (m, 2H), 7.20-7.27 (m, 1H), 7.30-7.38 (m, 4H), 7.34-7.43 (m, 2H), 7.43-7.53 (m, 4H), 7.50-7.58 (m, 4H), 7.65-7.79 (m, 8H), 7.79-7.87 (m, 3H), 7.91 (m, 1H), 8.00-8.06 (m, 1H).

### Example 91

### Synthesis of Compound 481

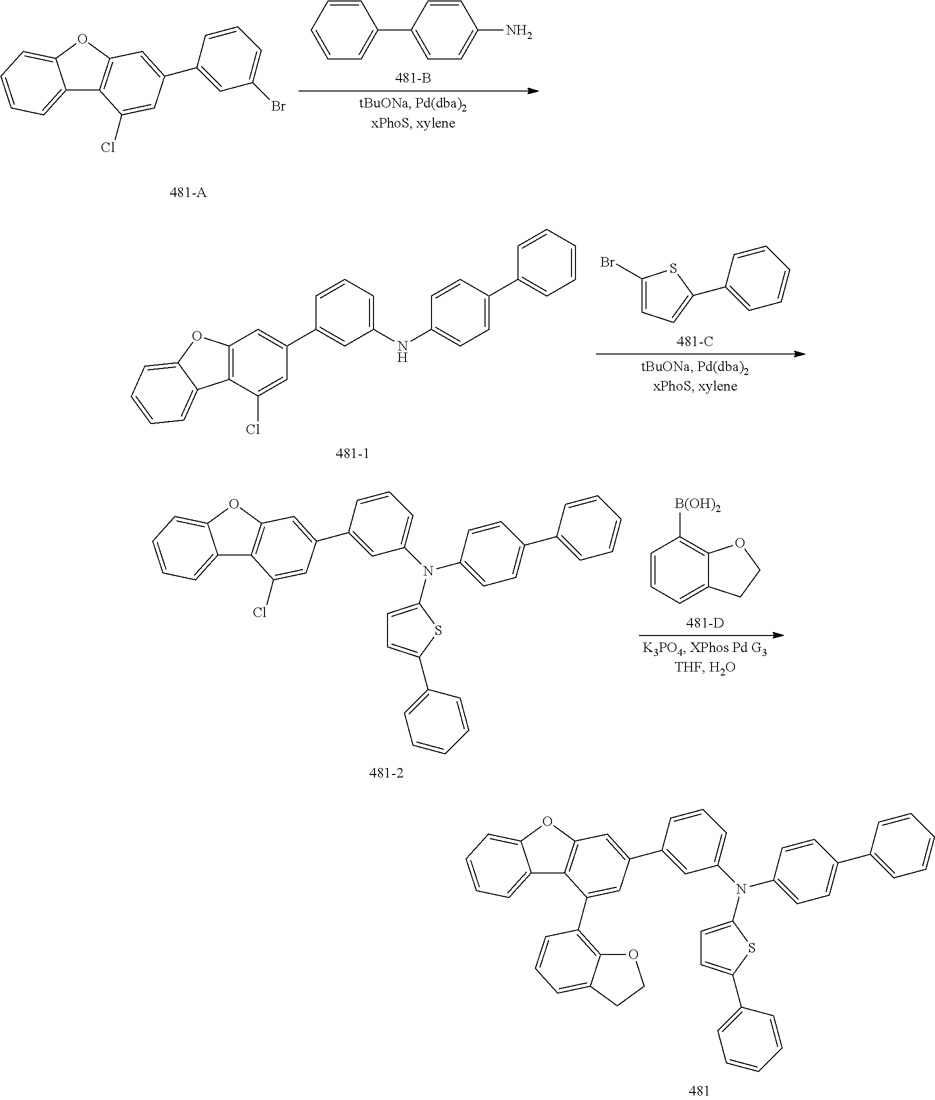

Except that the starting materials were replaced with 481-A, 481-B, 481-B and 481-D, everything else was the same as Example 88. LC MS: M/Z 687.22 (M+). Total yield of synthesis: 48%; HPLC purity: 99.9%.

¹H NMR (400 MHz, DMSO-d6) δ 3.23 (m, 2H), 4.53-4.69 (m, 2H), 6.00 (d, 1H), 6.93 (d, 1H), 7.20 (m, 2H), 7.30-7.66 (m, 17H), 7.69-7.80 (m, 3H), 7.77-7.89 (m, 4H), 7.98-8.06 (m, 1H).

### Example 92

### Synthesis of Compound 482

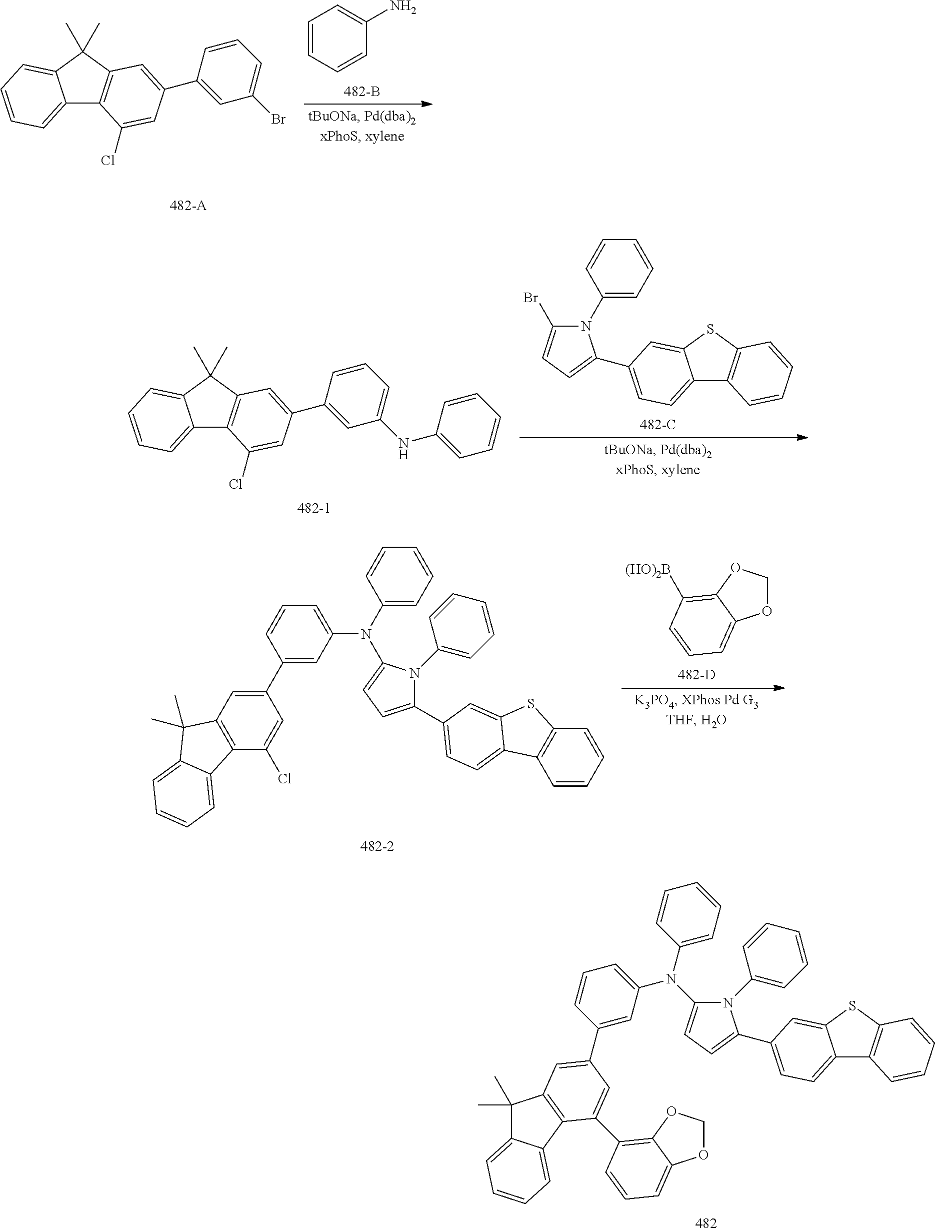

Except that the starting materials were replaced with 482-A, 482-B, 482-C and 482-D, everything else was the same as Example 88. LC MS: M/Z 804.28 (M+). Total yield of synthesis: 50%; HPLC purity: 99.9%.

¹H NMR (400 MHz, DMSO-d6) δ 1.69 (s, 6H), 5.75 (d, 1H), 6.33 (s, 2H), 6.54 (d, 1H), 6.95-7.08 (m, 1H), 7.04-7.13 (m, 4H), 7.18 (m, 1H), 7.20-7.29 (m, 3H), 7.31-7.67 (m, 13H), 7.77 (t, 1H), 7.85-7.95 (m, 3H), 8.06 (d, 1H), 8.17-8.25 (m, 2H), 8.45 (m, 1H).

### Example 93

### Synthesis of Compound 483

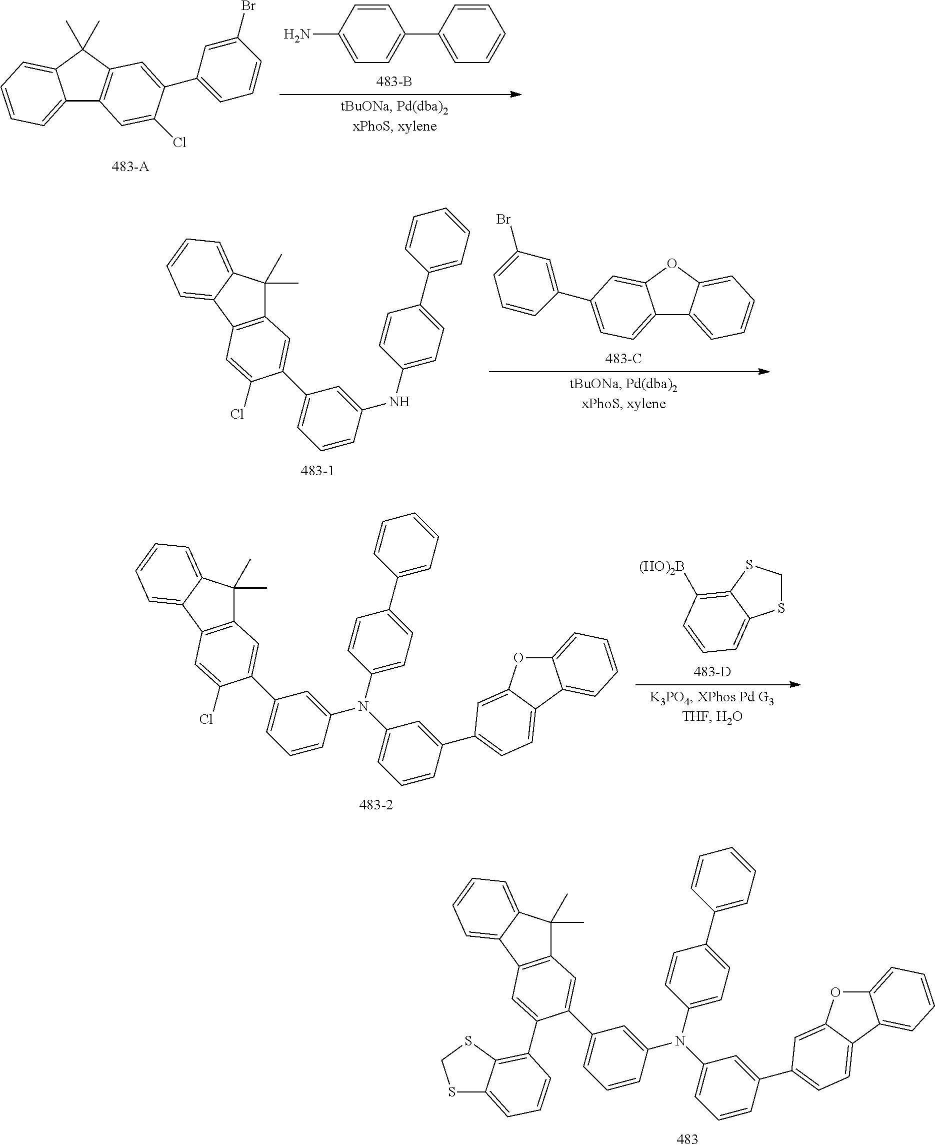

Except that the starting materials were replaced with 483-A, 483-B, 483-C and 483-D, everything else was the same as Example 88. LC MS: M/Z 831.26 (M+). Total yield of synthesis: 48%; HPLC purity: 99.9%.

¹H NMR (400 MHz, DMSO-d6) δ 1.69 (s, 6H), 4.29 (s, 2H), 7.14-7.27 (m, 5H), 7.27-7.58 (m, 18H), 7.66-7.80 (m, 7H), 7.84-7.94 (m, 2H), 7.94-8.02 (m, 1H), 8.37 (s, 1H).

### Example 94

### Synthesis of Compound 484

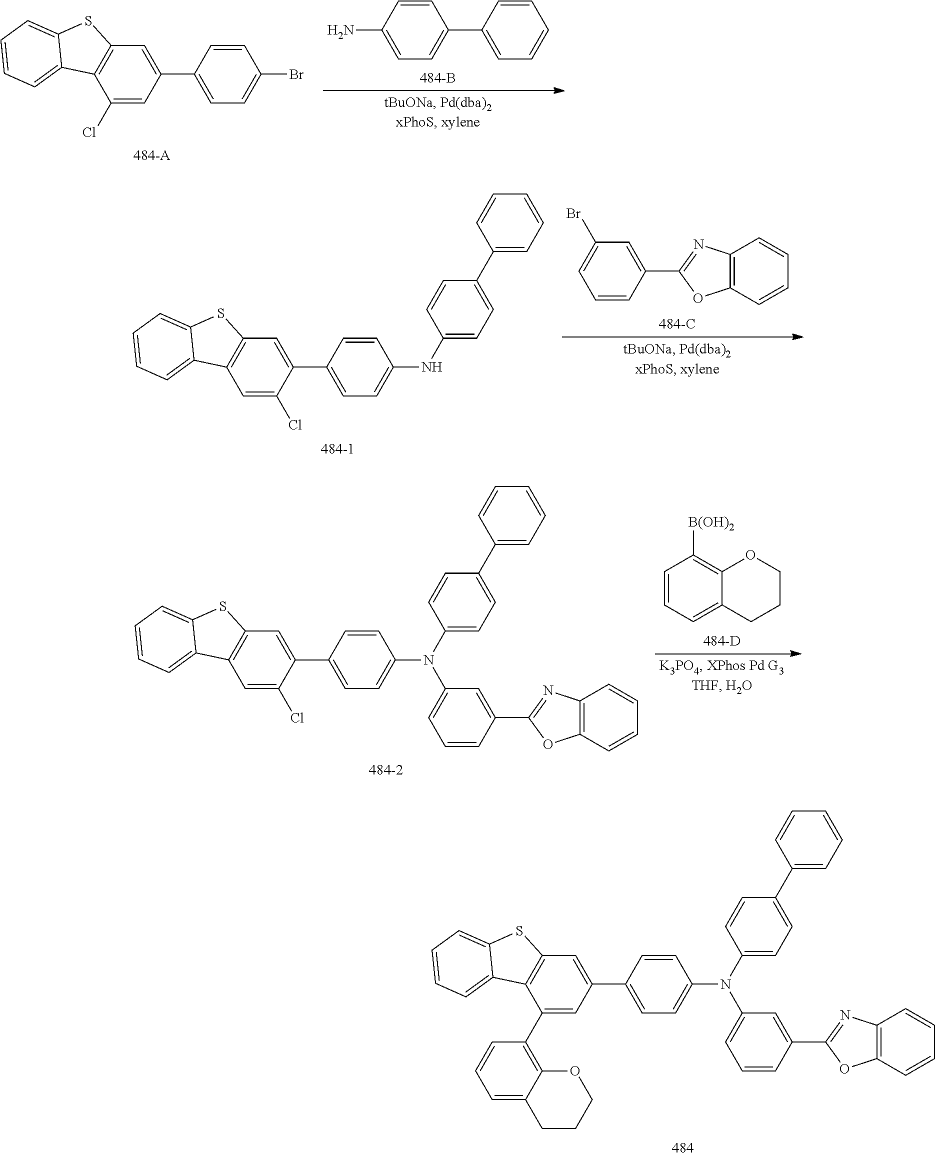

Except that the starting materials were replaced with 484-A, 484-B, 484-C and 484-D, everything else was the same as Example 88. LC MS: M/Z 792.24 (M+). Total yield of synthesis: 47%; HPLC purity: 99.9%.

¹H NMR (400 MHz, DMSO-d6) δ 1.96-2.10 (m, 2H), 2.78-2.88 (m, 2H), 4.22-4.30 (m, 2H), 7.18 (m, 1H), 7.30-7.69 (m, 22H), 7.69-7.80 (m, 3H), 7.87-7.95 (m, 1H), 8.01 (d, 1H), 8.30 (d, 1H), 8.45 (m, 1H).

### Example 95

### Synthesis of Compound 485

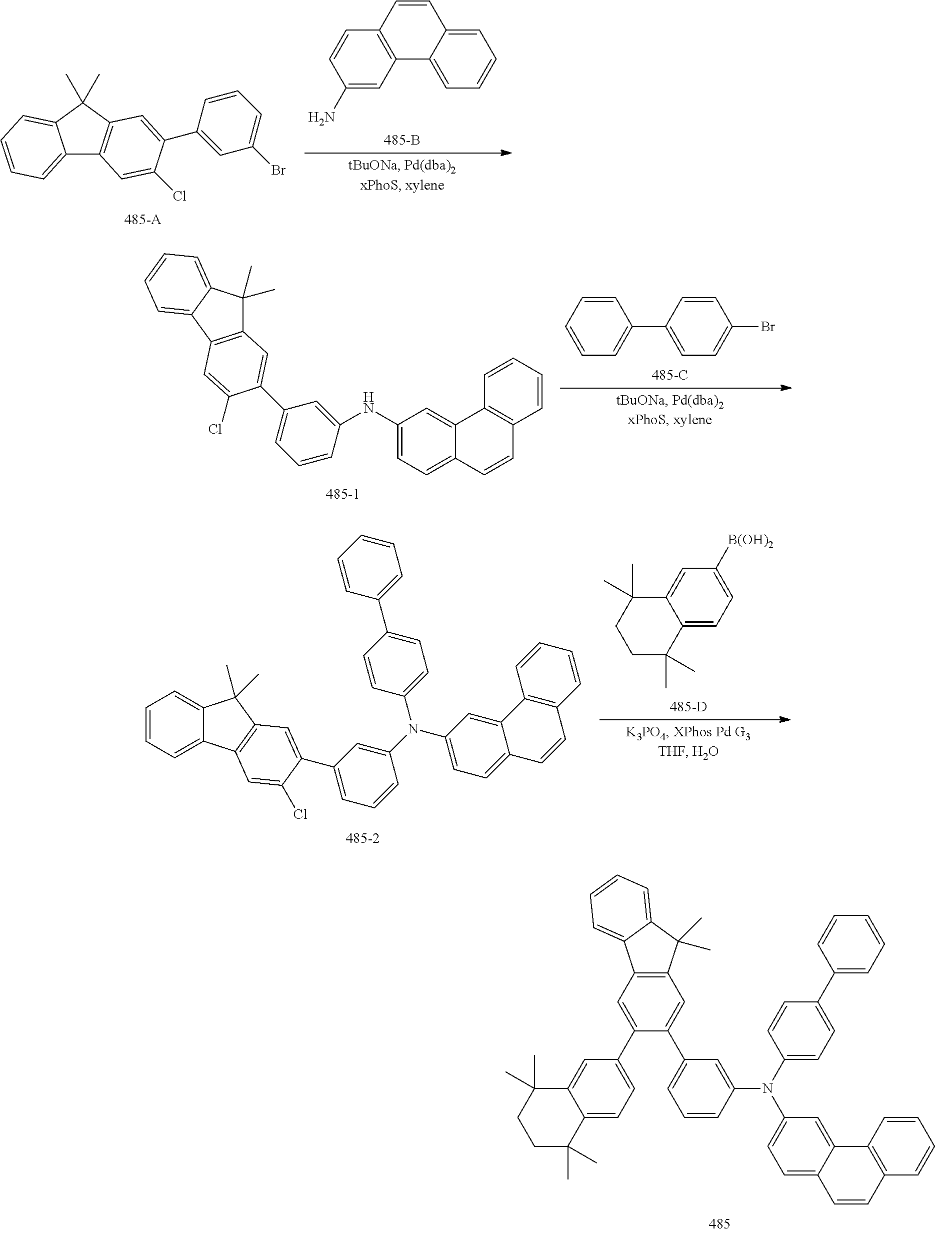

Except that the starting materials were replaced with 485-A, 485-B, 485-C and 485-D, everything else was the same as Example 88. LC MS: M/Z 799.42 (M+). Total yield of synthesis: 46%; HPLC purity: 99.9%.

¹H NMR (400 MHz, DMSO-d6) δ 1.22 (d, 12H), 1.48 (s, 4H), 1.69 (s, 6H), 6.67 (m, 1H), 7.18 (m, 1H), 7.19-7.27 (m, 2H), 7.30-7.80 (m, 20H), 7.80-7.94 (m, 3H), 7.99-8.07 (m, 1H), 8.27 (d, 1H), 8.37 (s, 1H), 8.73-8.81 (m, 1H).

### Example 96

### Synthesis of Compound 486

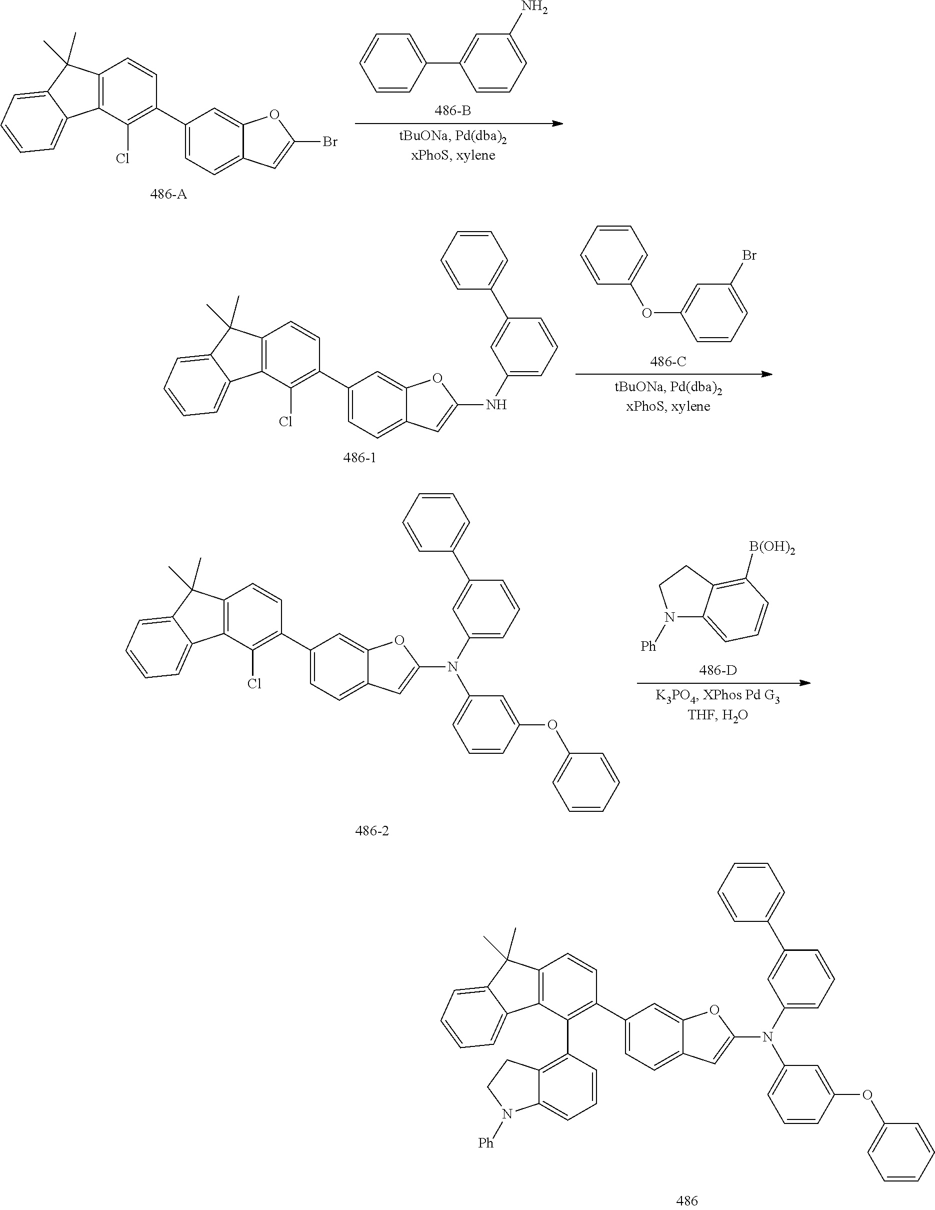

Except that the starting materials were replaced with 486-A, 486-B, 486-C and 486-D, everything else was the same as Example 88. LC MS: M/Z 838.36 (M+). Total yield of synthesis: 49%; HPLC purity: 99.9%.

¹H NMR (400 MHz, DMSO-d6) δ 1.69 (s, 6H), 3.18 (m, 1H), 3.30 (m, 1H), 4.14 (t, 2H), 6.44 (m, 1H), 6.70 (m, 1H), 6.80 (m, 1H), 6.87 (t, 1H), 6.99-7.27 (m, 7H), 7.27-7.38 (m, 4H), 7.34-7.44 (m, 6H), 7.39-7.53 (m, 7H), 7.48-7.57 (m, 1H), 7.70 (dd, 1H), 7.74-7.95 (m, 6H).

### Example 97

### Synthesis of Compound 487

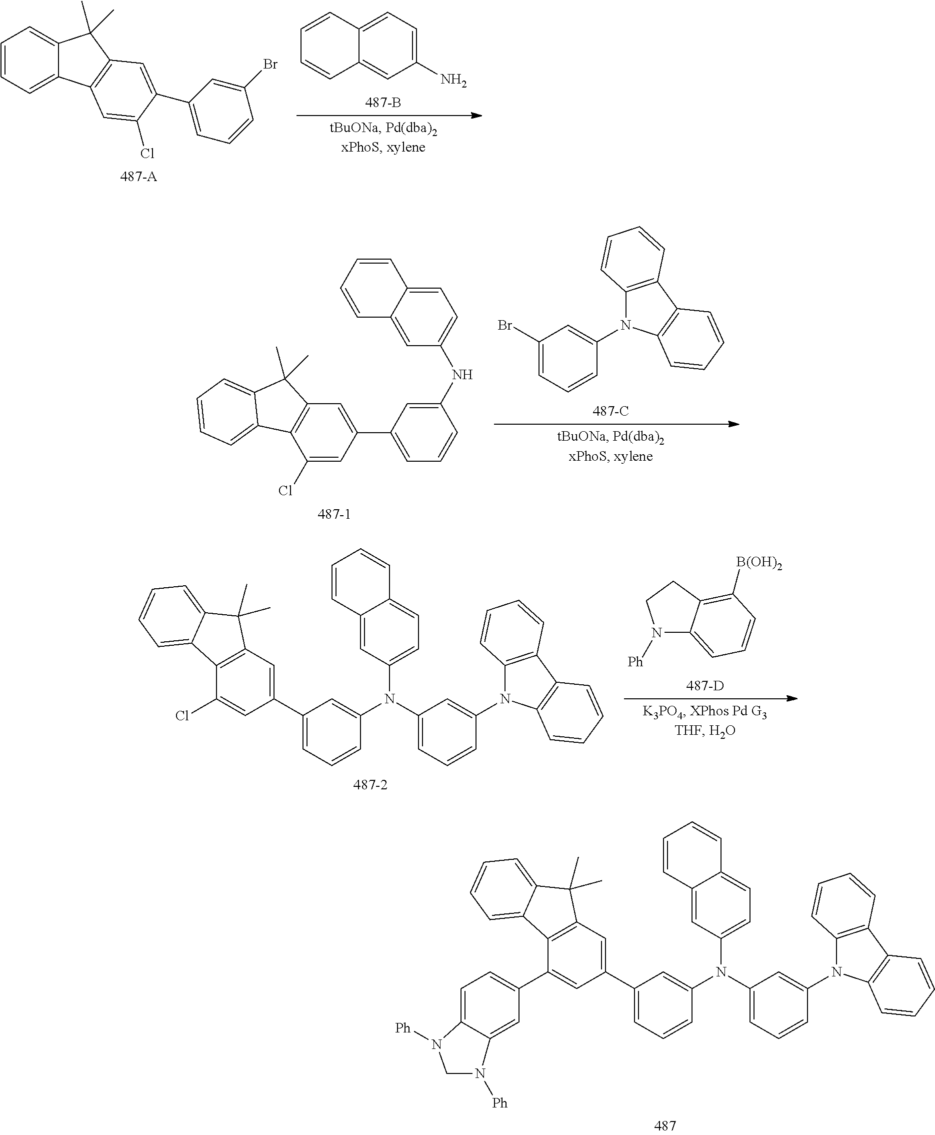

Except that the starting materials were replaced with 487-A, 487-B, 487-C and 487-D, everything else was the same as Example 88. LC MS: M/Z 922.40 (M+). Total yield of synthesis: 47%; HPLC purity: 99.9%.

¹H NMR (400 MHz, DMSO-d6) δ 1.69 (s, 6H), 4.83 (s, 2H), 7.00-7.10 (m, 3H), 7.08-7.14 (m, 1H), 7.14 (m, 1H), 7.14-7.27 (m, 3H), 7.27-7.50 (m, 21H), 7.50-7.58 (m, 2H), 7.59-7.67 (m, 2H), 7.67-7.78 (m, 3H), 7.74-7.83 (m, 2H), 7.85-7.91 (m, 1H), 8.06 (d, 1H), 8.16-8.24 (m, 2H).

### Example 98

### Synthesis of Compound 488

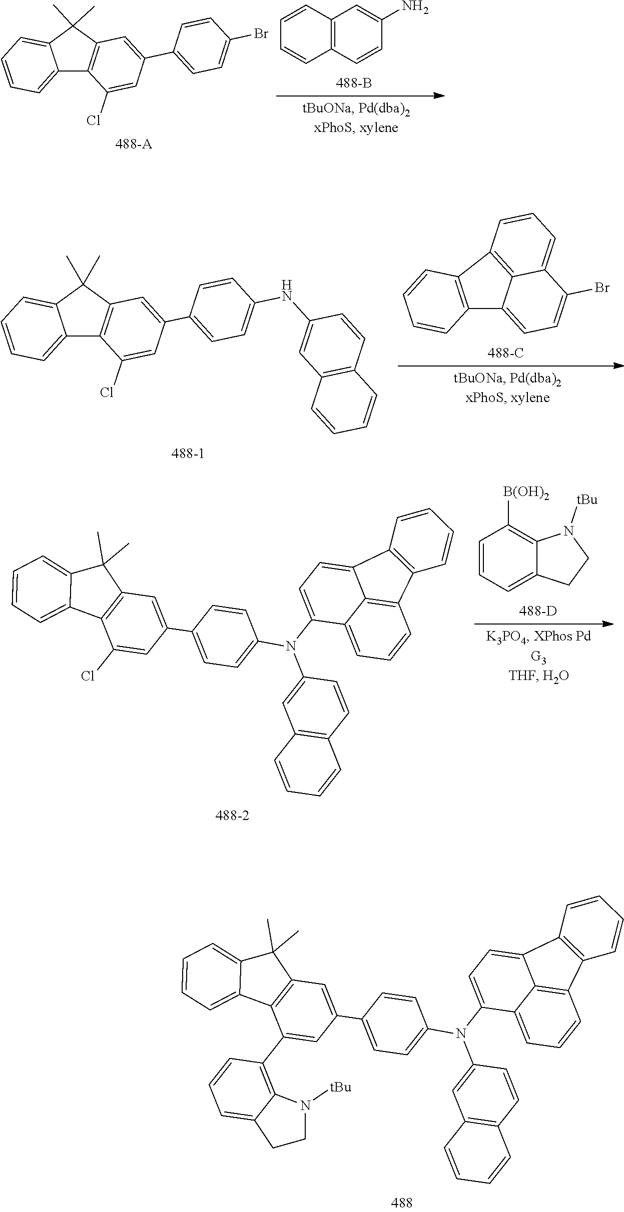

Except that the starting materials were replaced with 488-A, 488-B, 488-C and 488-D, everything else was the same as Example 88. LC MS: M/Z 784.38 (M+). Total yield of synthesis: 47%; HPLC purity: 99.9%.

¹H NMR (400 MHz, DMSO-d6) δ 1.32 (s, 8H), 1.69 (s, 6H), 2.98 (m, 2H), 3.61 (t, 2H), 7.04 (m, 1H), 7.11 (t, 1H), 7.23-7.35 (m, 1H), 7.31-7.39 (m, 4H), 7.39-7.50 (m, 5H), 7.50-7.59 (m, 5H), 7.67-7.82 (m, 8H), 7.85-7.91 (m, 1H), 8.00 (m, 1H), 8.06 (d, 1H), 8.22-8.31 (m, 2H).

### Example 99

### Synthesis of Compound 489

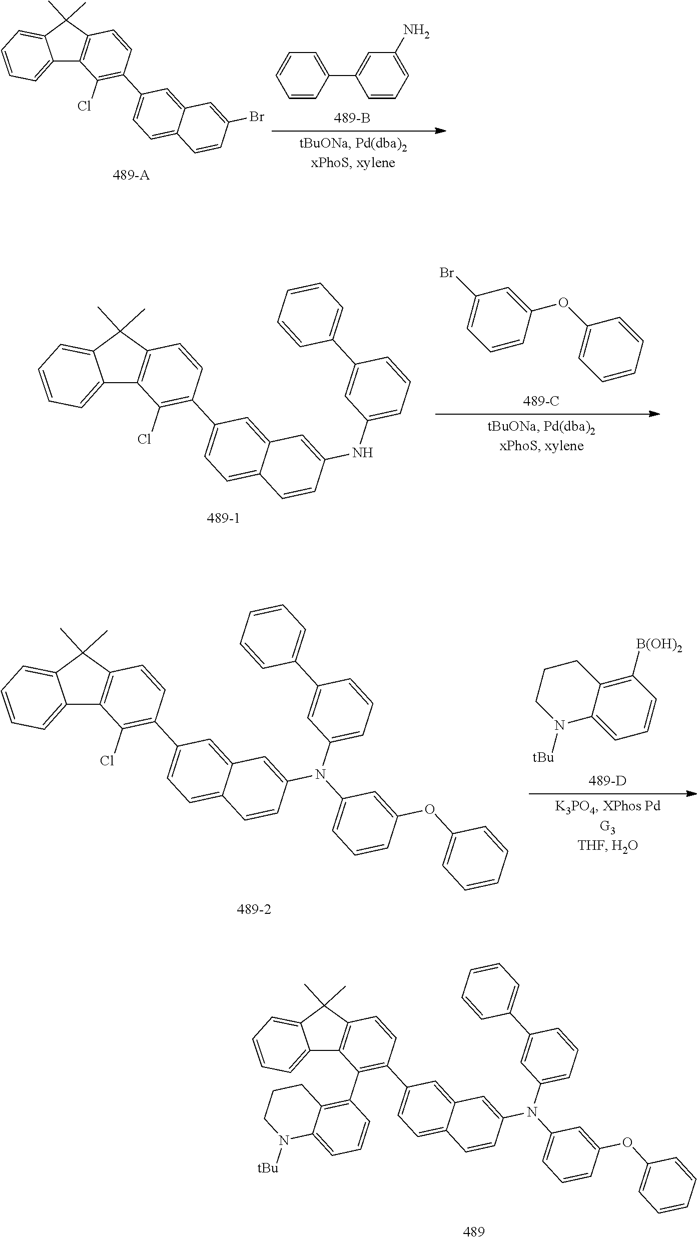

Except that the starting materials were replaced with 489-A, 489-B, 489-C and 489-D, everything else was the same as Example 88. LC MS: M/Z 842.42 (M+). Total yield of synthesis: 47%; HPLC purity: 99.9%.

¹H NMR (400 MHz, DMSO-d6) δ 1.32 (s, 8H), 1.69 (s, 6H), 1.96 (m, 2H), 2.64-2.86 (m, 2H), 3.37 (m, 2H), 6.44 (m, 1H), 6.73-6.84 (m, 2H), 6.87 (t, 1H), 6.99-7.06 (m, 2H), 7.06-7.27 (m, 9H), 7.30-7.59 (m, 14H), 7.72-7.79 (m, 2H), 7.82 (d, 1H), 7.83-7.92 (m, 2H).

### Example 100

### Synthesis of Compound 490

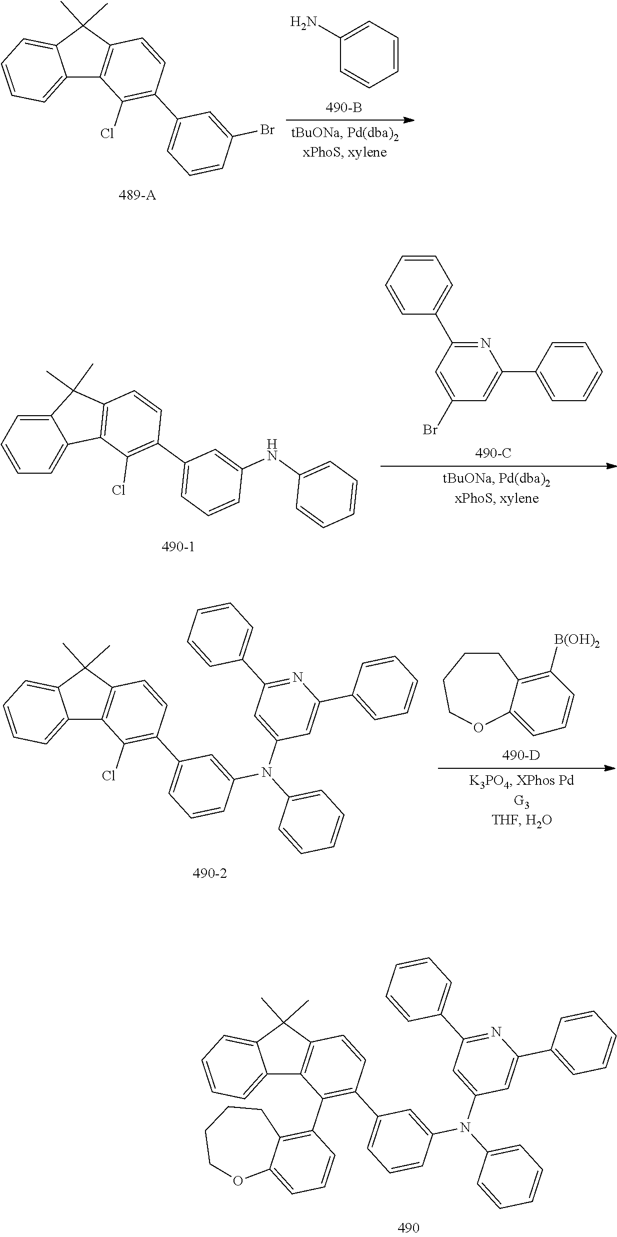

Except that the starting materials were replaced with 490-A, 490-B, 490-C and 490-D, everything else was the same as Example 88. LC MS: M/Z 736.35 (M+). Total yield of synthesis: 49%; HPLC purity: 99.9%.

¹H NMR (400 MHz, DMSO-d6) δ 1.69 (s, 6H), 1.63-1.83 (m, 3H), 2.78-2.88 (m, 2H), 4.14-4.22 (m, 2H), 6.68 (s, 2H), 7.00 (m, 1H), 7.04-7.12 (m, 3H), 7.14-7.50 (m, 12H), 7.50-7.59 (m, 5H), 7.77 (t, 1H), 7.82 (d, 1H), 7.84-7.91 (m, 2H), 8.24-8.32 (m, 4H).

### Example 101

### Synthesis of Compound 491

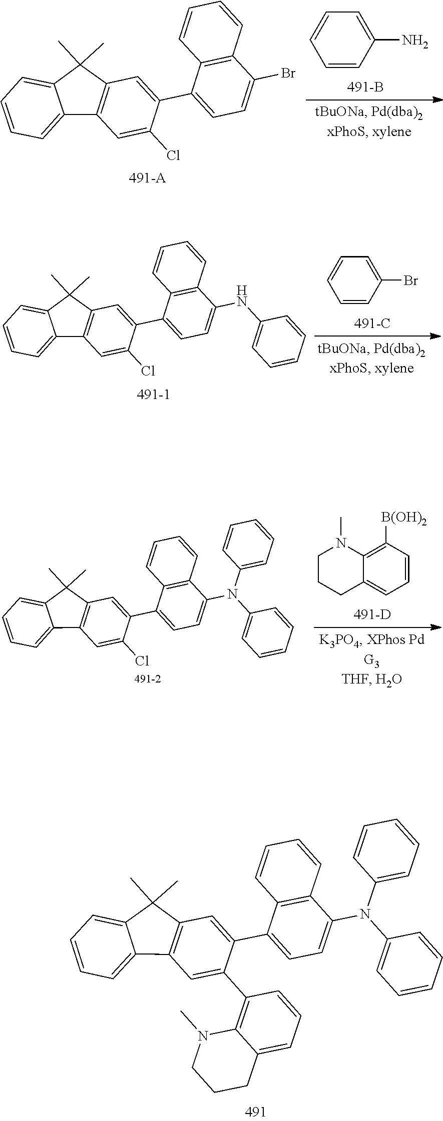

Except that the starting materials were replaced with 491-A, 491-B, 491-C and 491-D, everything else was the same as Example 88. LC MS: M/Z 632.32 (M+). Total yield of synthesis: 49%; HPLC purity: 99.9%.

¹H NMR (400 MHz, DMSO-d6) δ 1.69 (s, 6H), 1.96 (m, 2H), 2.55-2.81 (m, 2H), 2.85 (s, 3H), 3.37 (m, 2H), 6.95-7.12 (m, 8H), 7.19-7.31 (m, 5H), 7.31-7.50 (m, 5H), 7.50-7.57 (m, 1H), 7.76 (s, 1H), 7.84-7.92 (m, 1H), 8.00 (m, 1H), 8.16-8.26 (m, 1H), 8.37 (s, 1H), 8.90-9.00 (m, 1H).

### Example 102

### Synthesis of Compound 492

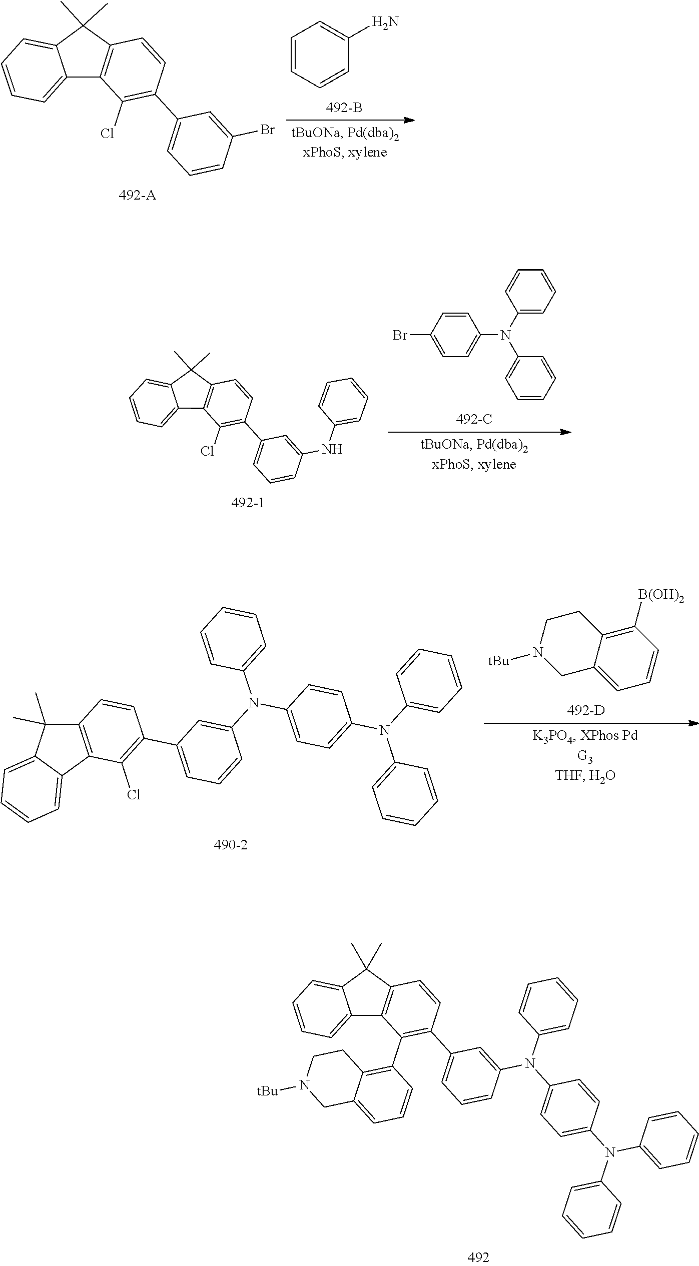

Except that the starting materials were replaced with 492-A, 492-B, 492-C and 492-D, everything else was the same as Example 88. LC MS: M/Z 791.42 (M+). Total yield of synthesis: 49%; HPLC purity: 99.9%.

¹H NMR (400 MHz, DMSO-d6) δ 1.02 (s, 9H), 1.69 (s, 6H), 2.77-2.89 (m, 2H), 3.26 (m, 1H), 3.48 (m, 1H), 4.34 (m, 1H), 4.50 (m, 1H), 6.95-7.04 (m, 3H), 7.04-7.14 (m, 10H), 7.14-7.29 (m, 8H), 7.30-7.50 (m, 4H), 7.50-7.57 (m, 1H), 7.71-7.92 (m, 6H).

### Example 103

### Synthesis of Compound 493

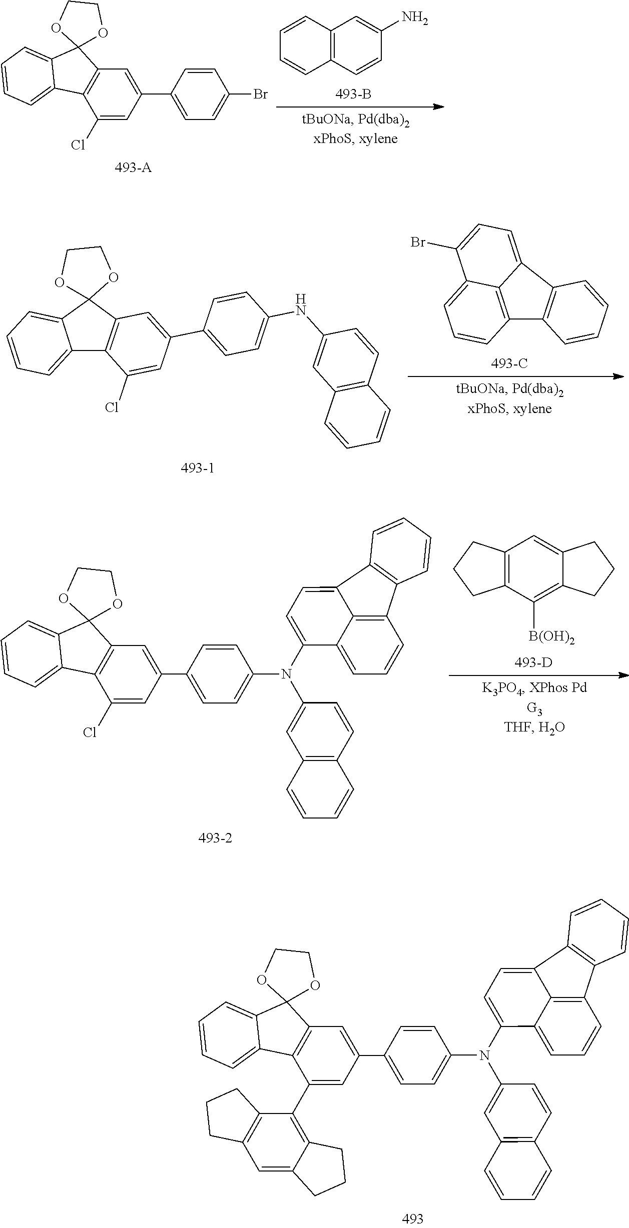

Except that the starting materials were replaced with 493-A, 493-B, 493-C and 493-D, everything else was the same as Example 88. LC MS: M/Z 797.33 (M+). Total yield of synthesis: 48%; HPLC purity: 99.9%.

¹H NMR (400 MHz, DMSO-d6) δ 1.93-2.08 (m, 4H), 2.63-2.81 (m, 4H), 2.84 (m, 4H), 3.43-3.54 (m, 2H), 3.54-3.65 (m, 2H), 7.05-7.14 (m, 2H), 7.30-7.39 (m, 4H), 7.39-7.50 (m, 4H), 7.50-7.60 (m, 5H), 7.64-7.83 (m, 8H), 7.88 (m, 1H), 8.06 (d, 1H), 8.26 (m, 2H).

### Example 104

### Synthesis of Compound 494

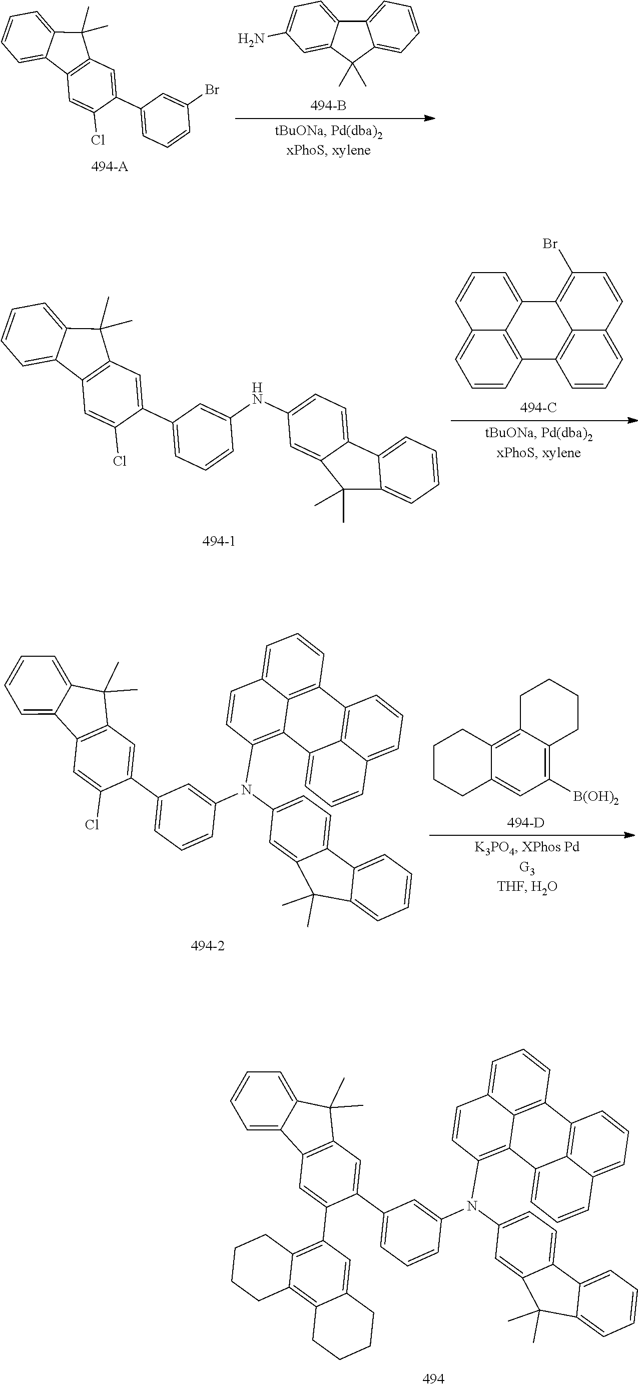

Except that the starting materials were replaced with 494-A, 494-B, 494-C and 494-D, everything else was the same as Example 88. LC MS: M/Z 911.45 (M+). Total yield of synthesis: 50%; HPLC purity: 99.9%.

¹H NMR (400 MHz, DMSO-d6) δ 1.40-1.54 (m, 2H), 1.66-1.93 (m, 19H), 2.37-2.45 (m, 1H), 2.62-2.75 (m, 6H), 6.95-7.01 (m, 1H), 7.13-7.22 (m, 3H), 7.23 (m, 1H), 7.30-7.57 (m, 12H), 7.62-7.70 (m, 3H), 7.77 (m, 2H), 7.81-7.92 (m, 3H), 8.16-8.24 (m, 3H), 8.37 (s, 1H).

### Example 105

### Synthesis of Compound 495

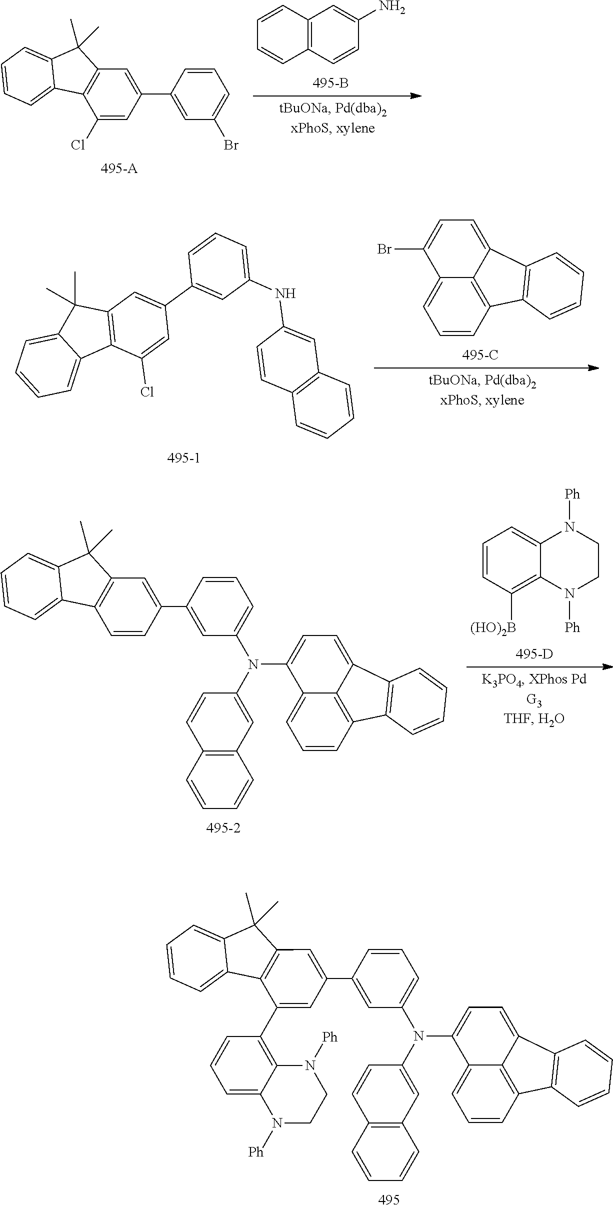

Except that the starting materials were replaced with 495-A, 495-B, 495-C and 495-D, everything else was the same as Example 88. LC MS: M/Z 895.39 (M+). Total yield of synthesis: 49%; HPLC purity: 99.9%.

¹H NMR (400 MHz, DMSO-d6) δ 1.69 (s, 6H), 3.29 (s, 4H), 7.02-7.11 (m, 3H), 7.11 (t, 1H), 7.14-7.27 (m, 3H), 7.29-7.50 (m, 16H), 7.50-7.59 (m, 3H), 7.67-7.82 (m, 8H), 7.85-7.93 (m, 2H), 8.06 (d, 1H), 8.22-8.31 (m, 2H).

### Example 106

### Synthesis of Compound 496

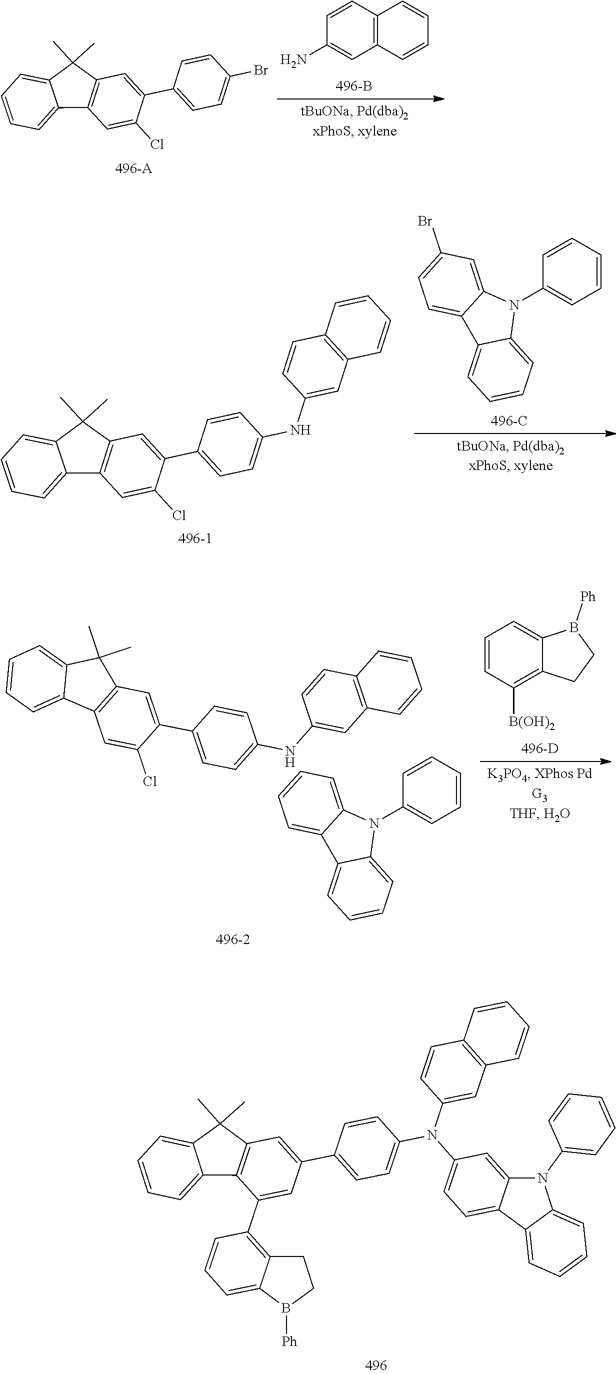

Except that the starting materials were replaced with 496-A, 496-B, 496-C and 496-D, everything else was the same as Example 88. LC MS: M/Z 842.38 (M+). Total yield of synthesis: 49%; HPLC purity: 99.9%.

¹H NMR (400 MHz, DMSO-d6) δ 1.69 (s, 6H), 1.70 (m, 2H), 2.58-2.64 (m, 1H), 7.11 (t, 1H), 7.19 (m, 1H), 7.23-7.30 (m, 2H), 7.30-7.67 (m, 24H), 7.67-7.74 (m, 1H), 7.71-7.78 (m, 2H), 7.74-7.82 (m, 2H), 7.82-7.91 (m, 2H), 8.06 (d, 1H), 8.17-8.27 (m, 2H).

### Example 107

### Synthesis of Compound 497

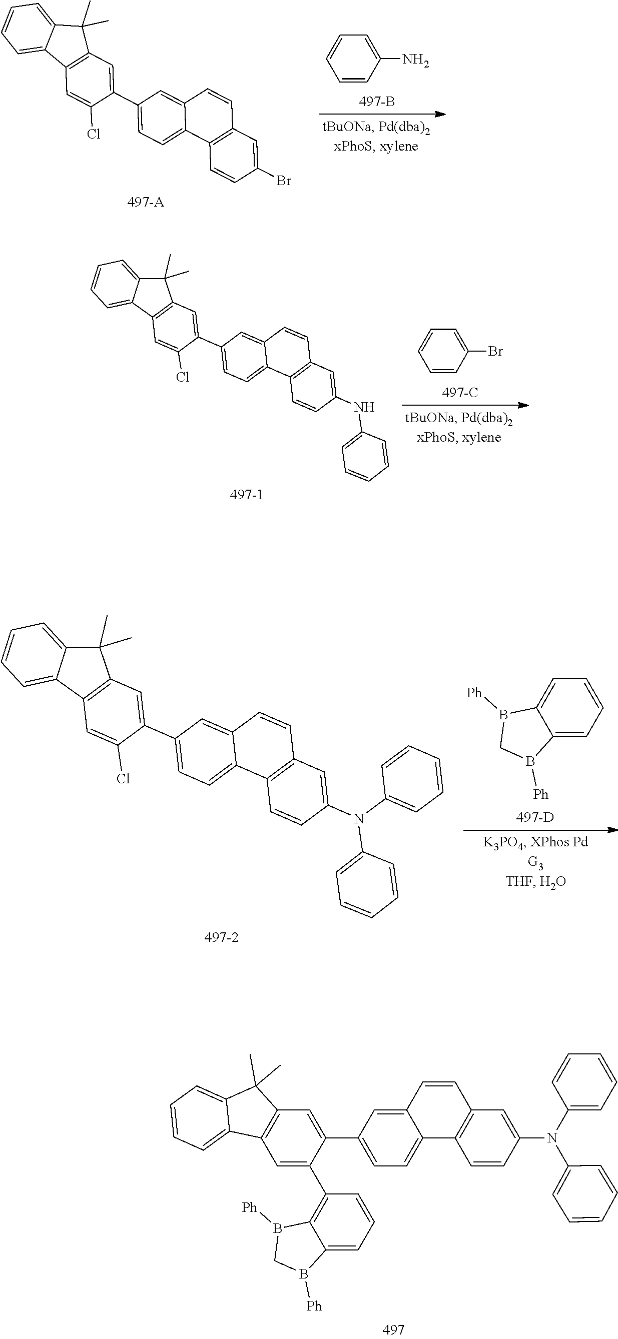

Except that the starting materials were replaced with 497-A, 497-B, 497-C and 497-D, everything else was the same as Example 88. LC MS: M/Z 801.37 (M+). Total yield of synthesis: 48%; HPLC purity: 99.9%.

¹H NMR (400 MHz, DMSO-d6) δ 1.40 (s, 2H), 1.69 (s, 6H), 6.95-7.04 (m, 2H), 7.04-7.12 (m, 4H), 7.19-7.29 (m, 4H), 7.35 (m, 3H), 7.42-7.50 (m, 2H), 7.50-7.62 (m, 8H), 7.66 (m, 1H), 7.70-7.79 (m, 6H), 7.81-7.92 (m, 2H), 8.07 (m, 1H), 8.37 (s, 1H), 8.84 (d, 1H), 8.95 (m, 1H), 9.07 (d, 1H).

### Example 108

### Synthesis of Compound 498

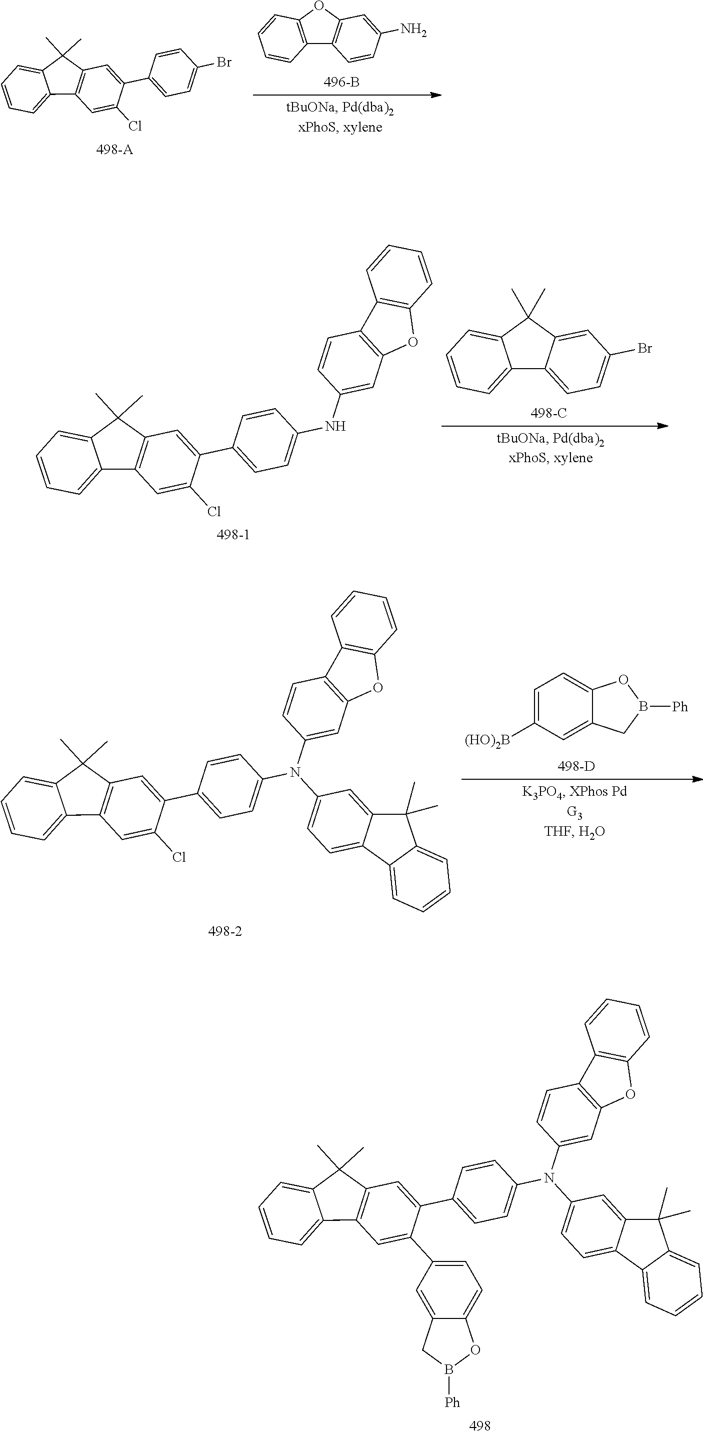

Except that the starting materials were replaced with 498-A, 498-B, 498-C and 498-D, everything else was the same as Example 88. LC MS: M/Z 835.36 (M+). Total yield of synthesis: 48%; HPLC purity: 99.9%.

¹H NMR (400 MHz, DMSO-d6) δ 1.69 (s, 12H), 6.91 (m, 1H), 6.94-7.01 (m, 2H), 7.26-7.41 (m, 9H), 7.41-7.61 (m, 12H), 7.68 (m, 1H), 7.71-7.79 (m, 3H), 7.82-7.92 (m, 3H), 7.94-8.01 (m, 1H), 8.03 (m, 1H), 8.37 (s, 1H).

### Example 109

### Synthesis of Compound 499

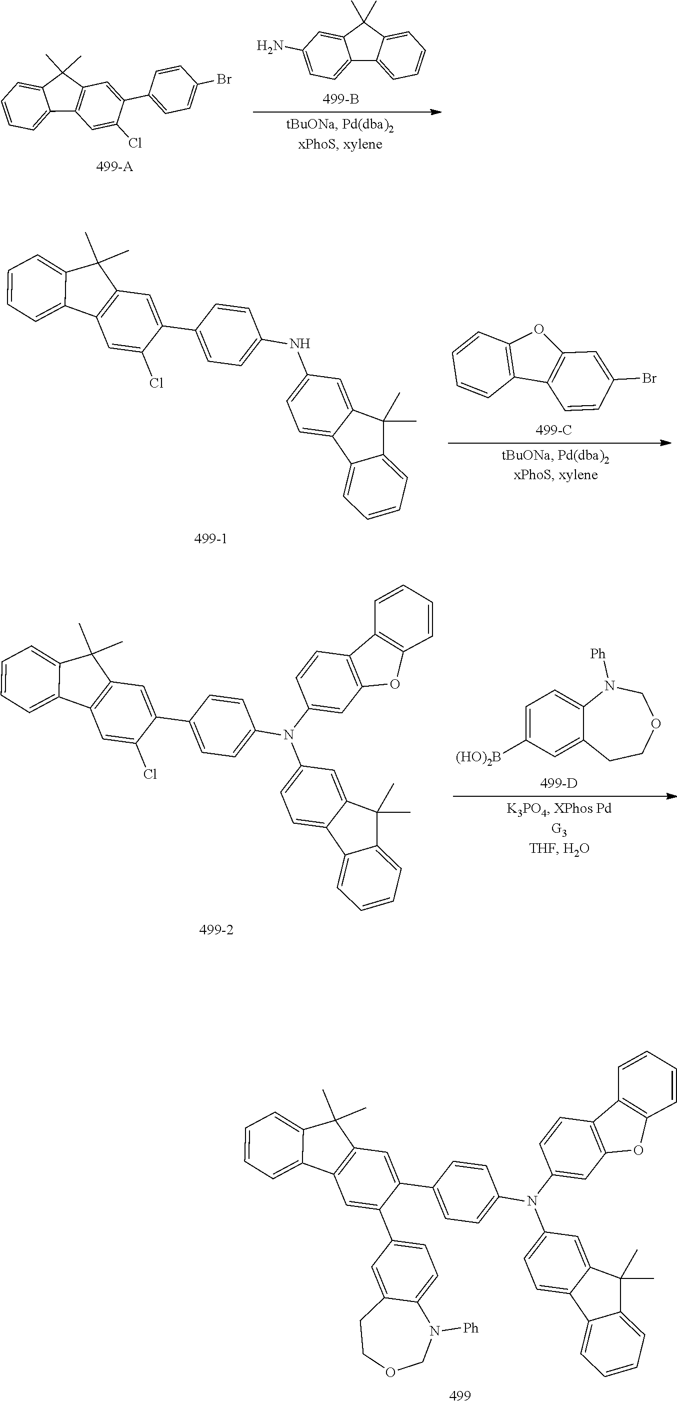

Except that the starting materials were replaced with 499-A, 499-B, 499-C and 499-D, everything else was the same as Example 88. LC MS: M/Z 866.39 (M+). Total yield of synthesis: 50%; HPLC purity: 99.9%.

¹H NMR (400 MHz, DMSO-d6) δ 1.69 (s, 12H), 2.72 (m, 2H), 3.70 (t, 2H), 5.14 (s, 2H), 6.91 (m, 1H), 6.95-7.01 (m, 1H), 7.06 (m, 1H), 7.26-7.50 (m, 15H), 7.50-7.58 (m, 5H), 7.68 (m, 1H), 7.71-7.78 (m, 2H), 7.82-7.91 (m, 3H), 7.95-8.01 (m, 1H), 8.03 (m, 1H), 8.37 (s, 1H).

### Example 110

### Synthesis of Compound 500

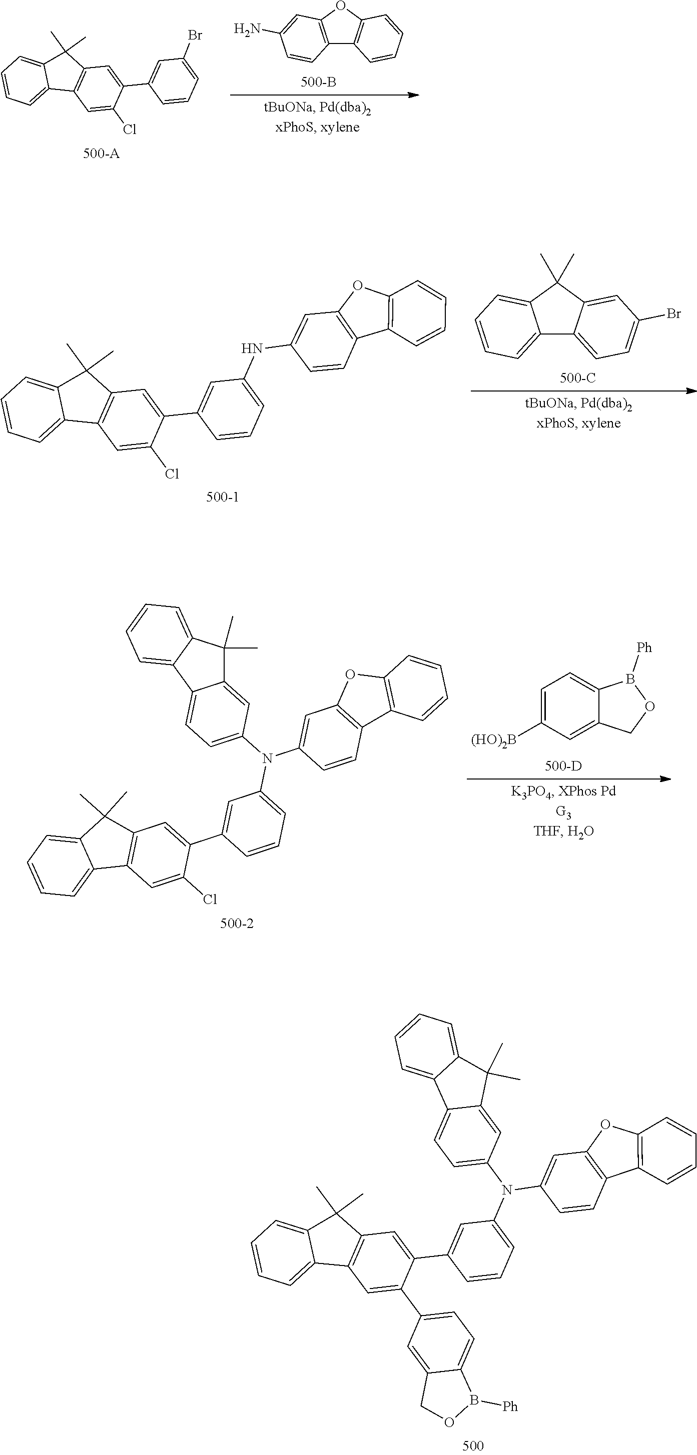

Except that the starting materials were replaced with 500-A, 500-B, 500-C and 500-D, everything else was the same as Example 88. LC MS: M/Z 835.36 (M+). Total yield of synthesis: 51%; HPLC purity: 99.9%.

¹H NMR (400 MHz, DMSO-d6) δ 1.69 (s, 12H), 5.12 (d, 2H), 6.91 (m, 1H), 6.95-7.01 (m, 1H), 7.18 (m, 1H), 7.23 (m, 1H), 7.26-7.50 (m, 9H), 7.50-7.60 (m, 5H), 7.65-7.73 (m, 2H), 7.71-7.80 (m, 4H), 7.82-7.91 (m, 4H), 7.94-8.01 (m, 2H), 8.03 (m, 1H), 8.37 (s, 1H).

### Example 111

### Synthesis of Compound 501

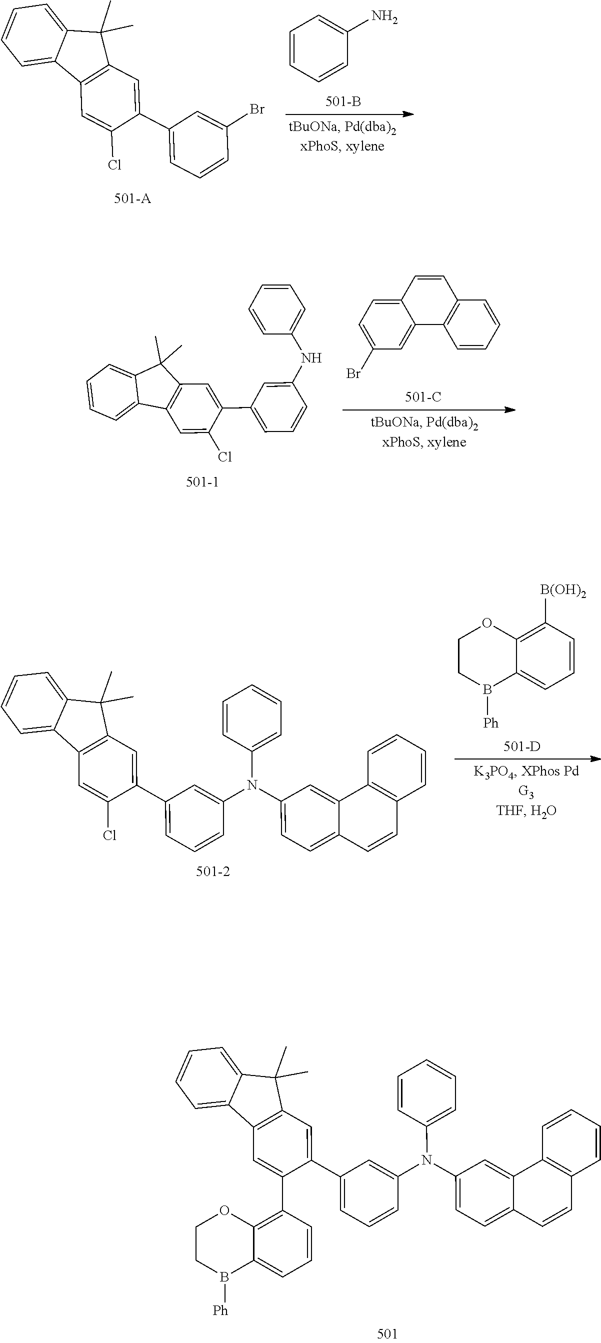

Except that the starting materials were replaced with 501-A, 501-B, 501-C and 501-D, everything else was the same as Example 88. LC MS: M/Z 743.34 (M+). Total yield of synthesis: 47%; HPLC purity: 99.9%.

¹H NMR (400 MHz, DMSO-d6) δ 1.69 (s, 6H), 1.80 (m, 2H), 4.00 (m, 2H), 7.00 (m, 1H), 7.04-7.12 (m, 2H), 7.14-7.29 (m, 5H), 7.35 (m, 2H), 7.37-7.50 (m, 2H), 7.50-7.80 (m, 12H), 7.81-7.94 (m, 4H), 8.00-8.06 (m, 1H), 8.27 (d, 1H), 8.37 (s, 1H), 8.74-8.80 (m, 1H).

### Example 112

### Synthesis of Compound 502

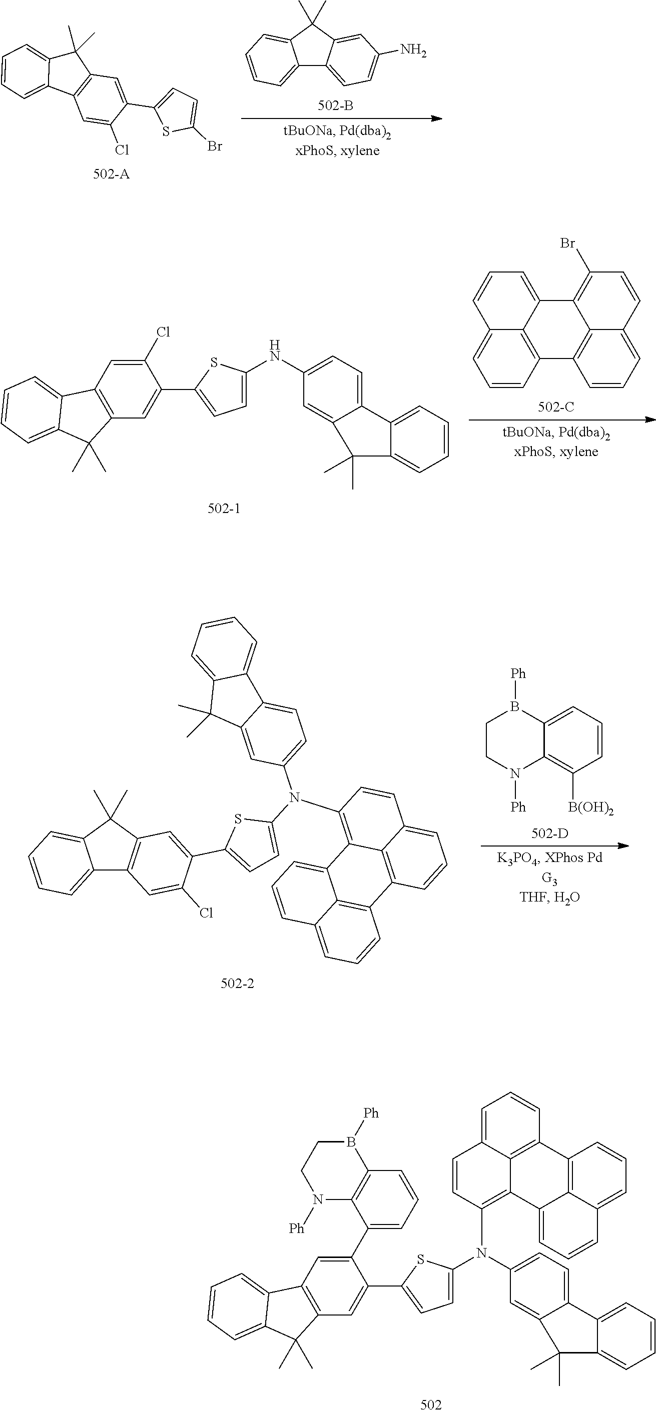

Except that the starting materials were replaced with 502-A, 502-B, 502-C and 502-D, everything else was the same as Example 88. LC MS: M/Z 1014.42 (M+). Total yield of synthesis: 47%; HPLC purity: 99.9%.

¹H NMR (400 MHz, DMSO-d6) δ 1.60 (m, 2H), 1.69 (s, 12H), 3.10 (m, 2H), 6.00 (d, 1H), 6.93 (d, 1H), 6.96-7.01 (m, 1H), 7.06 (m, 1H), 7.14-7.23 (m, 2H), 7.29-7.60 (m, 18H), 7.63-7.70 (m, 3H), 7.71-7.78 (m, 3H), 7.81 (m, 1H), 7.82-7.91 (m, 3H), 8.17-8.24 (m, 3H), 8.24 (m, 1H), 8.37 (s, 1H).

### Example 113

### Synthesis of Compound 504

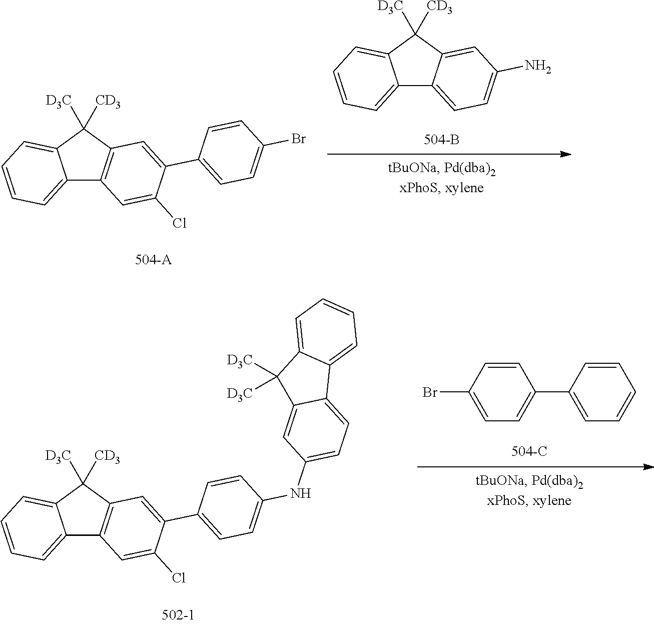 [SMILES: [2H]C([2H])([2H])C1(C([2H])([2H])[2H])c2ccccc2-c2cc(Cl)c(-c3ccc(Br)cc3)cc21 ; [2H]C([2H])([2H])C1(C([2H])([2H])[2H])c2ccccc2-c2ccc(N)cc21 ; [2H]C([2H])([2H])C1(C([2H])([2H])[2H])c2ccccc2-c2ccc(Nc3ccc(-c4cc5c(cc4Cl)-c4ccccc4C5(C([2H])([2H])[2H])C([2H])([2H])[2H])cc3)cc21 ; Brc1ccc(-c2ccccc2)cc1]

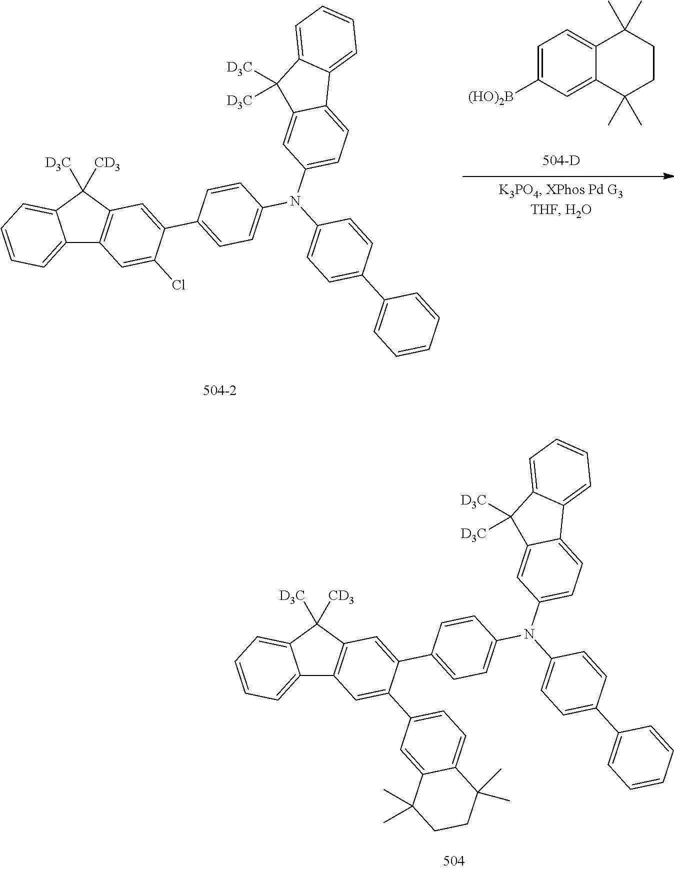 [SMILES: [2H]C([2H])([2H])C1(C([2H])([2H])[2H])c2ccccc2-c2ccc(N(c3ccc(-c4ccccc4)cc3)c3ccc(-c4cc5c(cc4Cl)-c4ccccc4C5(C([2H])([2H])[2H])C([2H])([2H])[2H])cc3)cc21 ; CC1(C)CCC(C)(C)c2cc(B(O)O)ccc21 ; [2H]C([2H])([2H])C1(C([2H])([2H])[2H])c2ccccc2-c2ccc(N(c3ccc(-c4ccccc4)cc3)c3ccc(-c4cc5c(cc4-c4ccc6c(c4)C(C)(C)CCC6(C)C)-c4ccccc4C5(C([2H])([2H])[2H])C([2H])([2H])[2H])cc3)cc21]

Except that the starting materials were replaced with 504-A, 504-B, 504-C and 504-D, everything else was the same as Example 88. LC MS: M/Z 827.52 (M+). Total yield of synthesis: 48%; HPLC purity: 99.9%.

¹H NMR (500 MHz, DMSO-d6) δ 8.01 (s, 1H), 7.72 (m, 1H), 7.69-7.61 (m, 4H), 7.61 (m, 1H), 7.58-7.50 (m, 4H), 7.49-7.27 (m, 11H), 7.27-7.18 (m, 4H), 7.21-7.14 (m, 2H), 6.92 (d, 1H), 1.74 (s, 4H), 1.28 (s, 12H).

### Example 114

### Synthesis of Compound 507

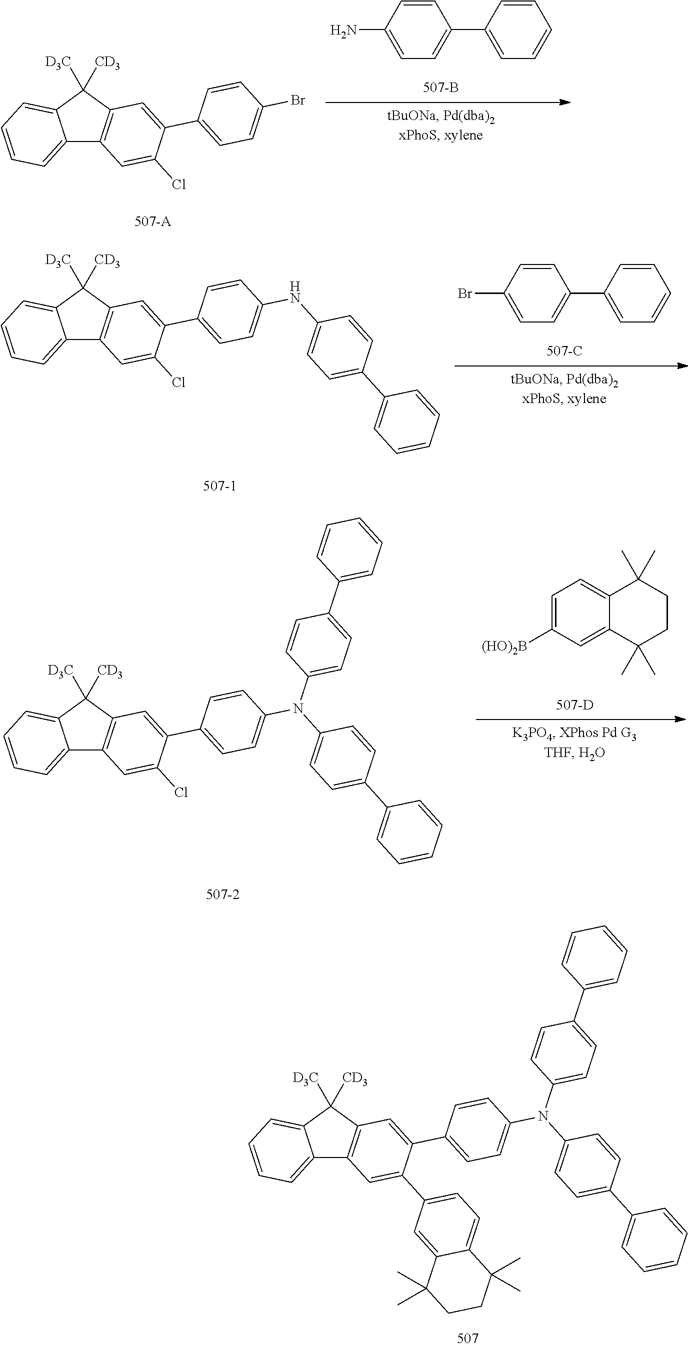

Except that the starting materials were replaced with 507-A, 507-B, 507-C and 507-D, everything else was the same as Example 88. LC MS: M/Z 781.46 (M+). Total yield of synthesis: 49%; HPLC purity: 99.9%.

¹H NMR (500 MHz, DMSO-d6) δ 8.01 (s, 1H), 7.69-7.63 (m, 5H), 7.61 (m, 1H), 7.58-7.51 (m, 6H), 7.49-7.27 (m, 8H), 7.24 (d, 1H), 7.21-7.14 (m, 6H), 1.74 (s, 3H), 1.74 (s, 4H), 1.28 (s, 12H).

### Example 115

### Synthesis of Compound 509

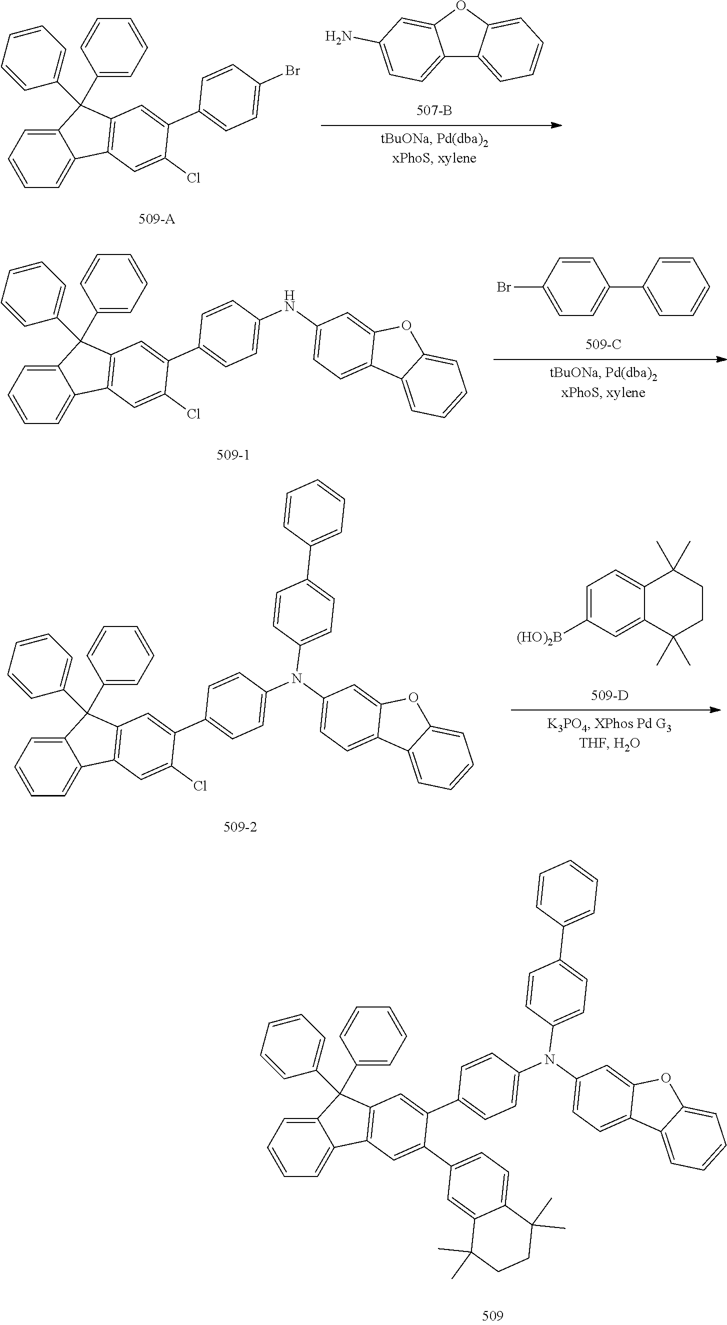

Except that the starting materials were replaced with 509-A, 509-B, 509-C and 509-D, everything else was the same as Example 88. LC MS: M/Z 914.21 (M+). Total yield of synthesis: 46%; HPLC purity: 99.9%.

¹H NMR (500 MHz, DMSO-d6) δ 8.04-7.98 (m, 2H), 7.94 (d, 1H), 7.66 (m, 3H), 7.58-7.51 (m, 6H), 7.51-7.32 (m, 10H), 7.32-7.14 (m, 11H), 7.14-7.07 (m, 5H), 6.87 (m, 1H), 1.74 (s, 4H), 1.28 (s, 12H).

### Example 116

### Synthesis of Compound 514

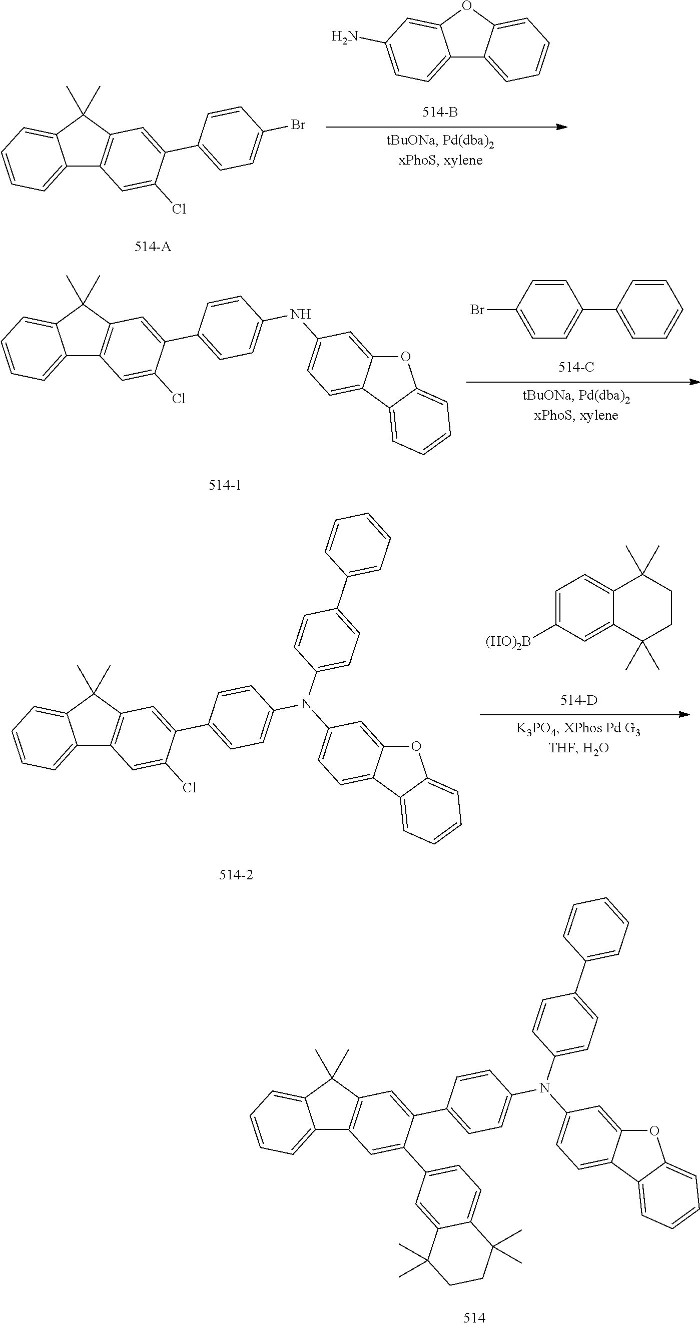

Except that the starting materials were replaced with 514-A, 514-B, 514-C and 514-D, everything else was the same as Example 88. LC MS: M/Z 789.40 (M+). Total yield of synthesis: 49%; HPLC purity: 99.9%.

¹H NMR (500 MHz, DMSO-d6) δ 8.03 (m, 1H), 7.98-7.91 (m, 2H), 7.69-7.63 (m, 2H), 7.63-7.57 (m, 2H), 7.57-7.51 (m, 5H), 7.51-7.38 (m, 6H), 7.41-7.29 (m, 4H), 7.24 (d, 1H), 7.21-7.14 (m, 4H), 7.11 (d, 1H), 6.87 (m, 1H), 1.74 (s, 4H), 1.59 (s, 6H), 1.28 (d, 12H).

### Example 117

### Synthesis of Compound 519

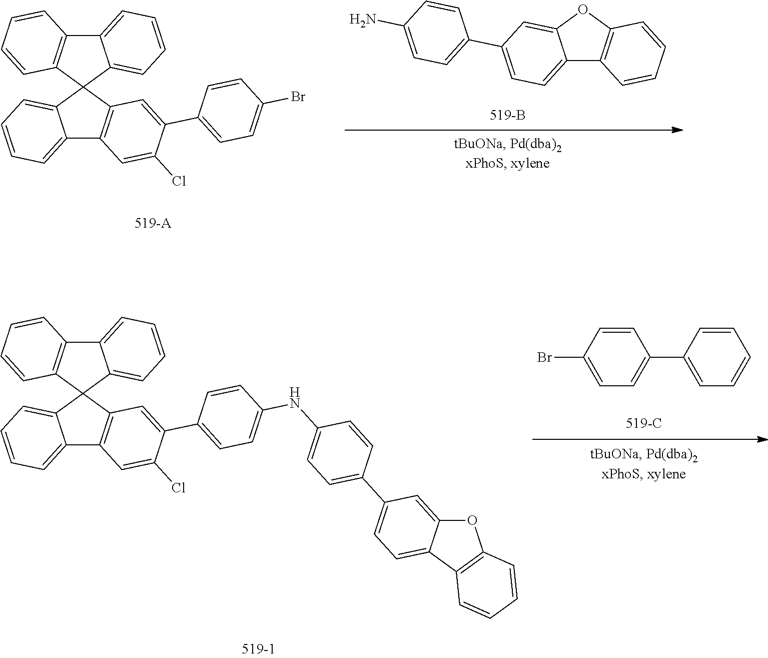 [SMILES: Clc1cc2c(cc1-c1ccc(Br)cc1)C1(c3ccccc3-c3ccccc31)c1ccccc1-2 ; Nc1ccc(-c2ccc3c(c2)oc2ccccc23)cc1 ; Clc1cc2c(cc1-c1ccc(Nc3ccc(-c4ccc5c(c4)oc4ccccc45)cc3)cc1)C1(c3ccccc3-c3ccccc31)c1ccccc1-2 ; Brc1ccc(-c2ccccc2)cc1]

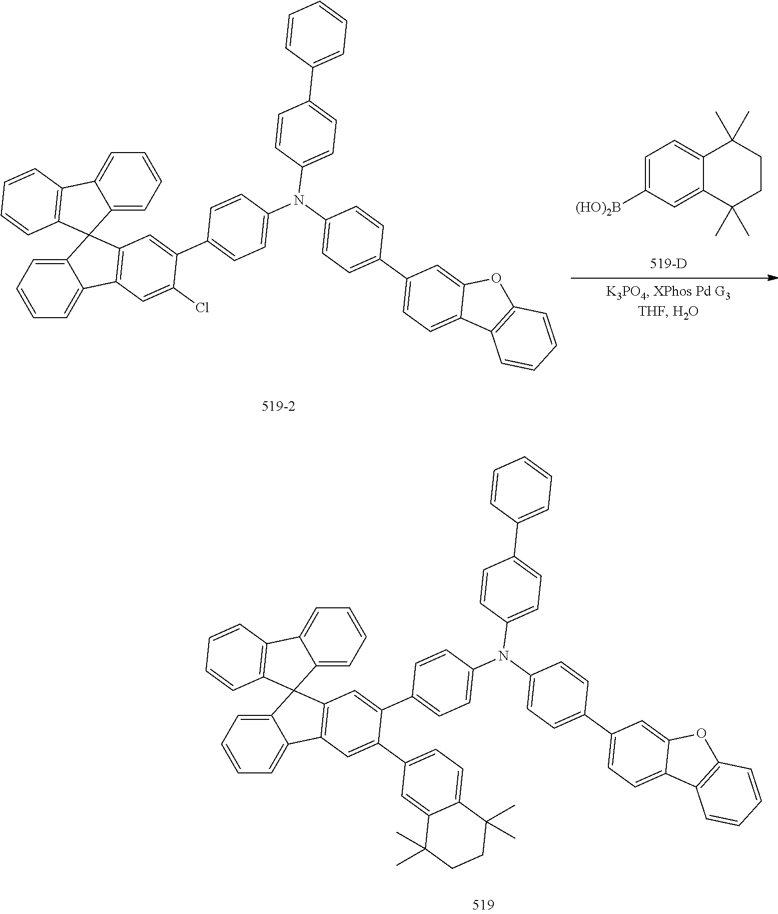 [SMILES: Clc1cc2c(cc1-c1ccc(N(c3ccc(-c4ccccc4)cc3)c3ccc(-c4ccc5c(c4)oc4ccccc45)cc3)cc1)C1(c3ccccc3-c3ccccc31)c1ccccc1-2 ; CC1(C)CCC(C)(C)c2cc(B(O)O)ccc21 ; CC1(C)CCC(C)(C)c2cc(-c3cc4c(cc3-c3ccc(N(c5ccc(-c6ccccc6)cc5)c5ccc(-c6ccc7c(c6)oc6ccccc67)cc5)cc3)C3(c5ccccc5-c5ccccc53)c3ccccc3-4)ccc21]

Except that the starting materials were replaced with 519-A, 519-B, 519-C and 519-D, everything else was the same as Example 88. LC MS: M/Z 987.44 (M+). Total yield of synthesis: 47%; HPLC purity: 99.9%.

¹H NMR (500 MHz, DMSO-d6) δ 8.06-8.00 (m, 2H), 7.90 (d, 1H), 7.84 (m, 2H), 7.73 (d, 1H), 7.69-7.63 (m, 3H), 7.65-7.59 (m, 2H), 7.59-7.21 (m, 21H), 7.21-7.14 (m, 6H), 6.91 (m, 1H), 6.82 (m, 2H), 1.74 (s, 4H), 1.28 (s, 12H).

### Example 118

### Synthesis of Compound 521

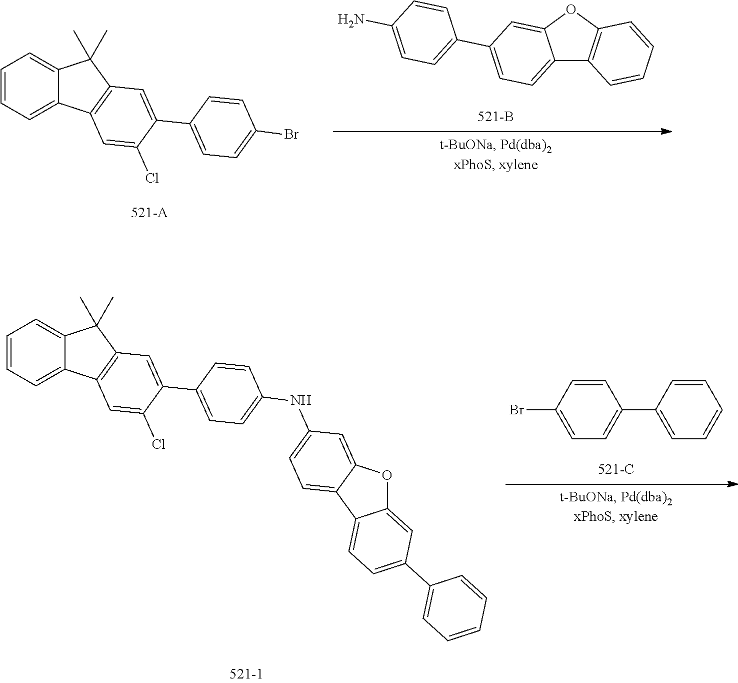 [SMILES: CC1(C)c2ccccc2-c2cc(Cl)c(-c3ccc(Br)cc3)cc21 ; Nc1ccc(-c2ccc3c(c2)oc2ccccc23)cc1 ; CC1(C)c2ccccc2-c2cc(Cl)c(-c3ccc(Nc4ccc5c(c4)oc4cc(-c6ccccc6)ccc45)cc3)cc21 ; Brc1ccc(-c2ccccc2)cc1]

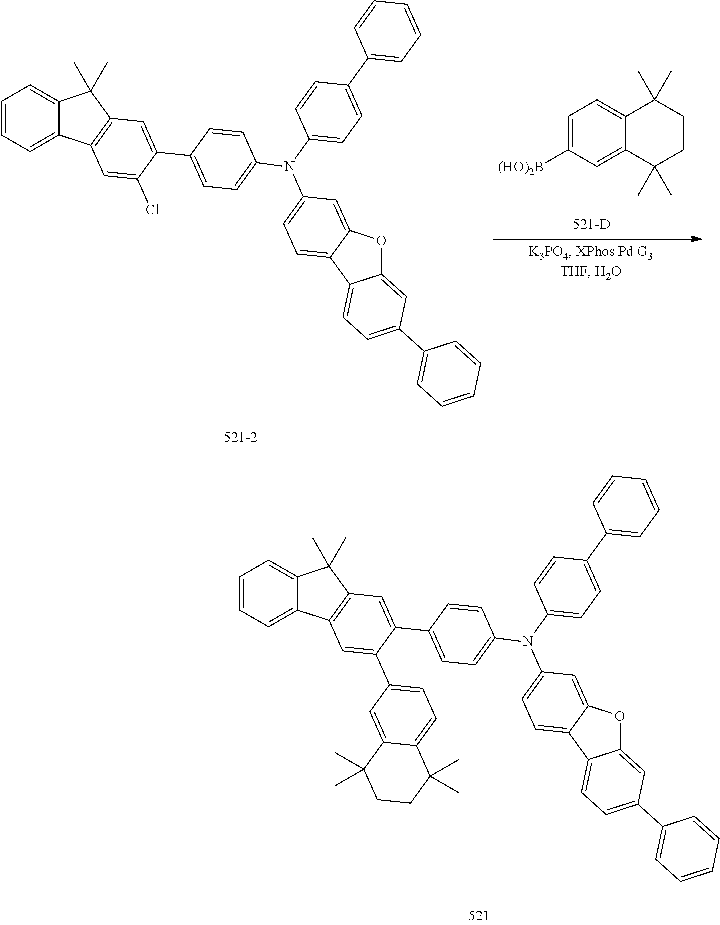 [SMILES: CC1(C)c2ccccc2-c2cc(Cl)c(-c3ccc(N(c4ccc(-c5ccccc5)cc4)c4ccc5c(c4)oc4cc(-c6ccccc6)ccc45)cc3)cc21 ; CC1(C)CCC(C)(C)c2cc(B(O)O)ccc21 ; CC1(C)CCC(C)(C)c2cc(-c3cc4c(cc3-c3ccc(N(c5ccc(-c6ccccc6)cc5)c5ccc6c(c5)oc5cc(-c7ccccc7)ccc56)cc3)C(C)(C)c3ccccc3-4)ccc21]

Except that the starting materials were replaced with 521-A, 521-B, 521-C and 521-D, everything else was the same as Example 88. LC MS: M/Z 865.43 (M+). Total yield of synthesis: 45%; HPLC purity: 99.9%.

¹H NMR (500 MHz, DMSO-d6) δ 8.06-8.00 (m, 2H), 7.90 (d, 1H), 7.84 (m, 2H), 7.73 (d, 1H), 7.69-7.63 (m, 3H), 7.65-7.59 (m, 2H), 7.59-7.21 (m, 22H), 7.21-7.14 (m, 6H), 6.91 (m, 1H), 6.82 (m, 2H), 1.74 (s, 4H), 1.28 (d, 12H).

### Example 119

### Synthesis of Compound 524

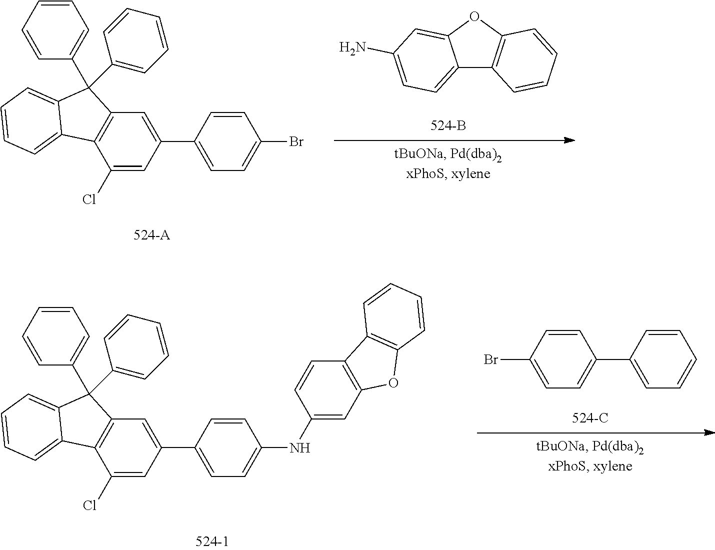 [SMILES: Clc1cc(-c2ccc(Br)cc2)cc2c1-c1ccccc1C2(c1ccccc1)c1ccccc1 ; Nc1ccc2c(c1)oc1ccccc12 ; Clc1cc(-c2ccc(Nc3ccc4c(c3)oc3ccccc34)cc2)cc2c1-c1ccccc1C2(c1ccccc1)c1ccccc1 ; Brc1ccc(-c2ccccc2)cc1]

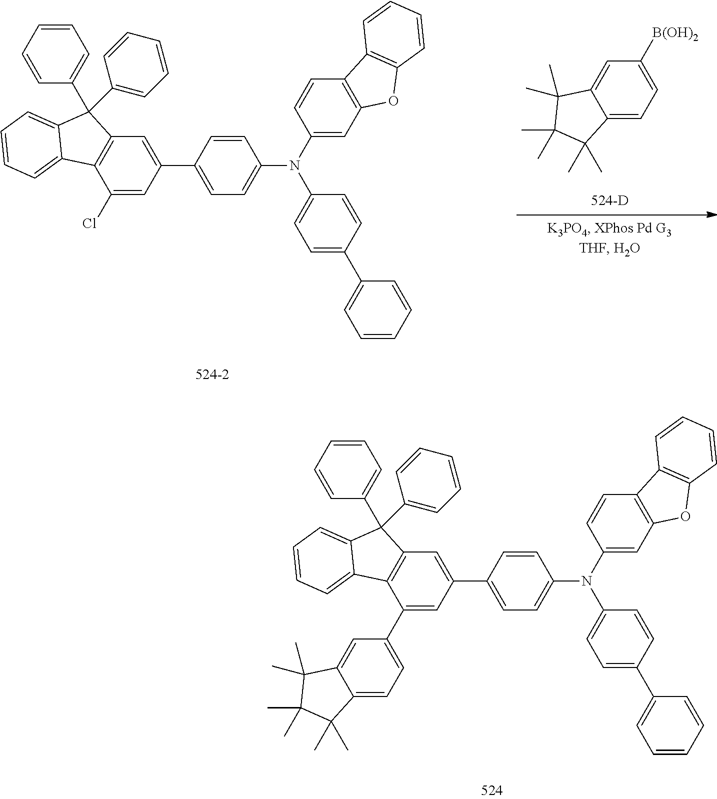 [SMILES: Clc1cc(-c2ccc(N(c3ccc(-c4ccccc4)cc3)c3ccc4c(c3)oc3ccccc34)cc2)cc2c1-c1ccccc1C2(c1ccccc1)c1ccccc1 ; CC1(C)c2ccc(B(O)O)cc2C(C)(C)C1(C)C ; CC1(C)c2ccc(-c3cc(-c4ccc(N(c5ccc(-c6ccccc6)cc5)c5ccc6c(c5)oc5ccccc56)cc4)cc4c3-c3ccccc3C4(c3ccccc3)c3ccccc3)cc2C(C)(C)C1(C)C]

Except that the starting materials were replaced with 524-A, 524-B, 524-C and 524-D, everything else was the same as Example 88. LC MS: M/Z 927.44 (M+). Total yield of synthesis: 44%; HPLC purity: 99.9%.

¹H NMR (500 MHz, DMSO-d6) δ 8.01 (m, 1H), 7.94 (d, 1H), 7.81 (d, 1H), 7.73 (m, 1H), 7.69-7.63 (m, 2H), 7.60-7.52 (m, 5H), 7.51-7.32 (m, 8H), 7.32-7.13 (m, 17H), 7.10 (m, 2H), 6.87 (m, 1H), 1.31 (s, 12H), 0.93 (s, 6H).

### Example 120

### Synthesis of Compound 526

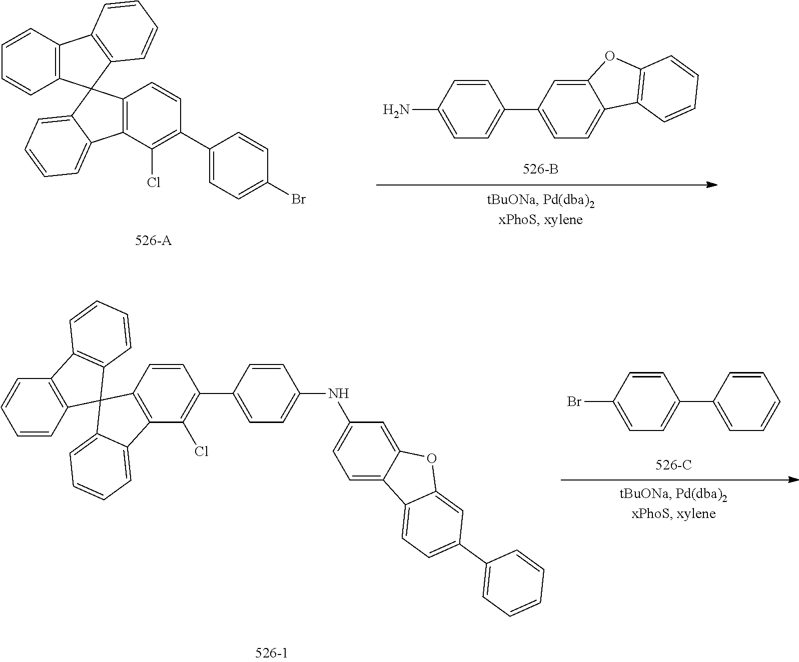 [SMILES: Clc1c(-c2ccc(Br)cc2)ccc2c1-c1ccccc1C21c2ccccc2-c2ccccc21 ; Nc1ccc(-c2ccc3c(c2)oc2ccccc23)cc1 ; Clc1c(-c2ccc(Nc3ccc4c(c3)oc3cc(-c5ccccc5)ccc34)cc2)ccc2c1-c1ccccc1C21c2ccccc2-c2ccccc21 ; Brc1ccc(-c2ccccc2)cc1]

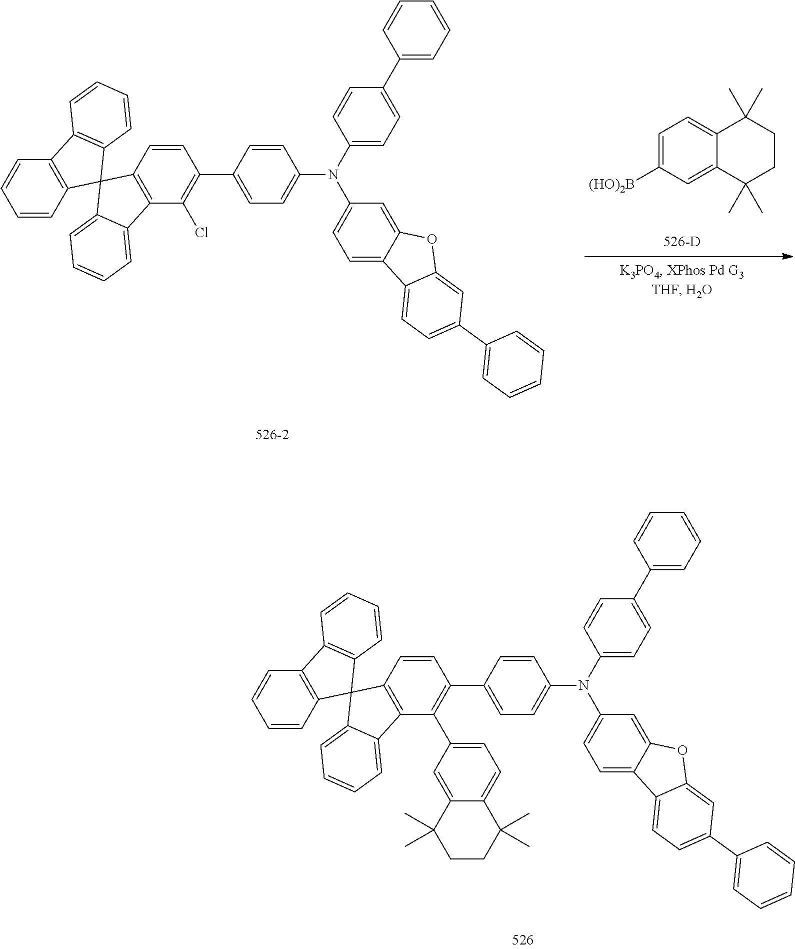 [SMILES: Clc1c(-c2ccc(N(c3ccc(-c4ccccc4)cc3)c3ccc4c(c3)oc3cc(-c5ccccc5)ccc34)cc2)ccc2c1-c1ccccc1C21c2ccccc2-c2ccccc21 ; CC1(C)CCC(C)(C)c2cc(B(O)O)ccc21 ; CC1(C)CCC(C)(C)c2cc(-c3c(-c4ccc(N(c5ccc(-c6ccccc6)cc5)c5ccc6c(c5)oc5cc(-c7ccccc7)ccc56)cc4)ccc4c3-c3ccccc3C43c4ccccc4-c4ccccc43)ccc21]

Except that the starting materials were replaced with 526-A, 526-B, 526-C and 526-D, everything else was the same as Example 88. LC MS: M/Z 987.44 (M+). Total yield of synthesis: 48%; HPLC purity: 99.9%.

¹H NMR (500 MHz, DMSO-d6) δ 7.93 (d, 1H), 7.84 (m, 3H), 7.75-7.69 (m, 2H), 7.69-7.51 (m, 10H), 7.48-7.21 (m, 16H), 7.20-7.14 (m, 4H), 7.14-7.06 (m, 2H), 6.88 (m, 1H), 6.82 (m, 2H), 1.74 (s, 4H), 1.28 (d, 12H).

### Example 121

### Synthesis of Compound 527

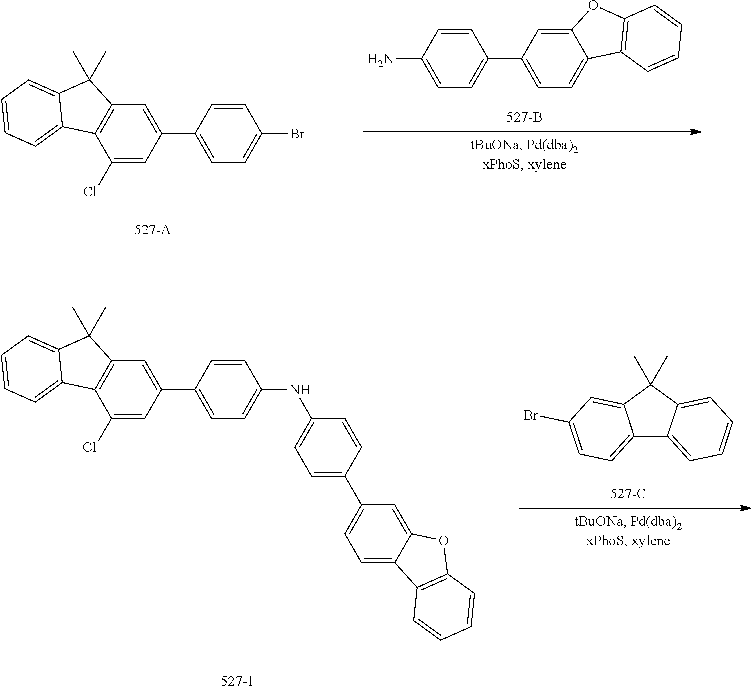 [SMILES: CC1(C)c2ccccc2-c2c(Cl)cc(-c3ccc(Br)cc3)cc21 ; Nc1ccc(-c2ccc3c(c2)oc2ccccc23)cc1 ; CC1(C)c2ccccc2-c2c(Cl)cc(-c3ccc(Nc4ccc(-c5ccc6c(c5)oc5ccccc56)cc4)cc3)cc21 ; CC1(C)c2ccccc2-c2ccc(Br)cc21]

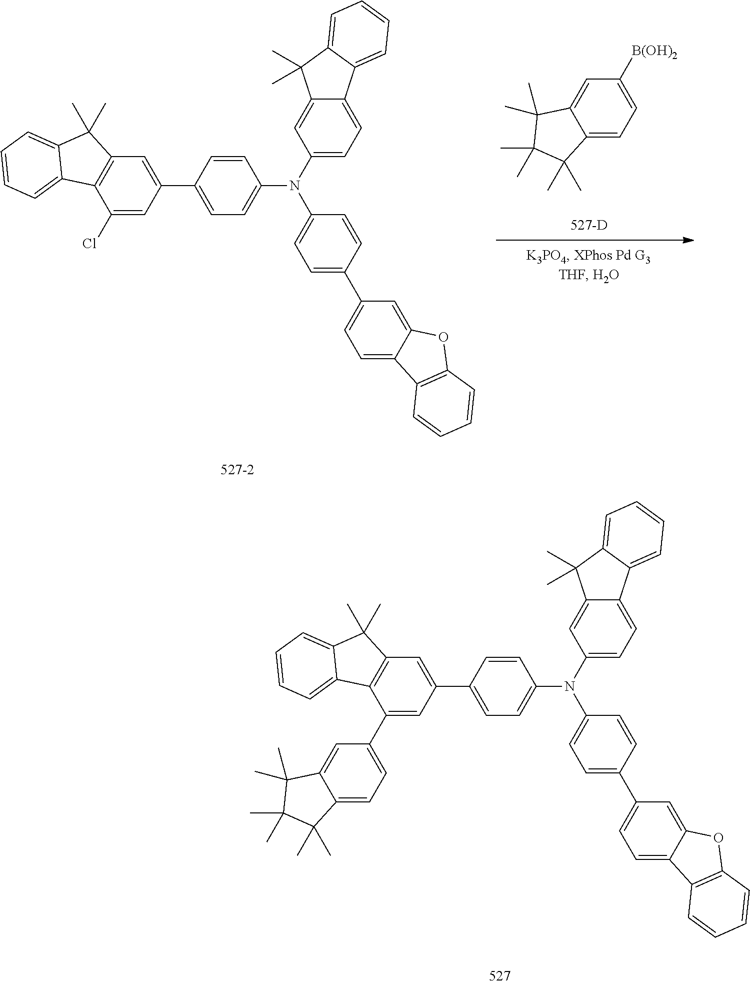 [SMILES: CC1(C)c2ccccc2-c2ccc(N(c3ccc(-c4cc(Cl)c5c(c4)C(C)(C)c4ccccc4-5)cc3)c3ccc(-c4ccc5c(c4)oc4ccccc45)cc3)cc21 ; CC1(C)c2ccc(B(O)O)cc2C(C)(C)C1(C)C ; CC1(C)c2ccccc2-c2ccc(N(c3ccc(-c4cc(-c5ccc6c(c5)C(C)(C)C(C)(C)C6(C)C)c5c(c4)C(C)(C)c4ccccc4-5)cc3)c3ccc(-c4ccc5c(c4)oc4ccccc45)cc3)cc21]

Except that the starting materials were replaced with 527-A, 527-B, 527-C and 527-D, everything else was the same as Example 88. LC MS: M/Z 919.48 (M+). Total yield of synthesis: 48%; HPLC purity: 99.9%.

¹H NMR (500 MHz, DMSO-d6) δ 8.06-8.01 (m, 1H), 7.89 (d, 1H), 7.80 (d, 1H), 7.75-7.66 (m, 4H), 7.65-7.27 (m, 16H), 7.25-7.14 (m, 6H), 7.09 (d, 1H), 6.96 (d, 1H), 1.60 (s, 12H), 1.31 (s, 12H), 0.93 (s, 6H).

### Example 122

### Synthesis of Compound 528

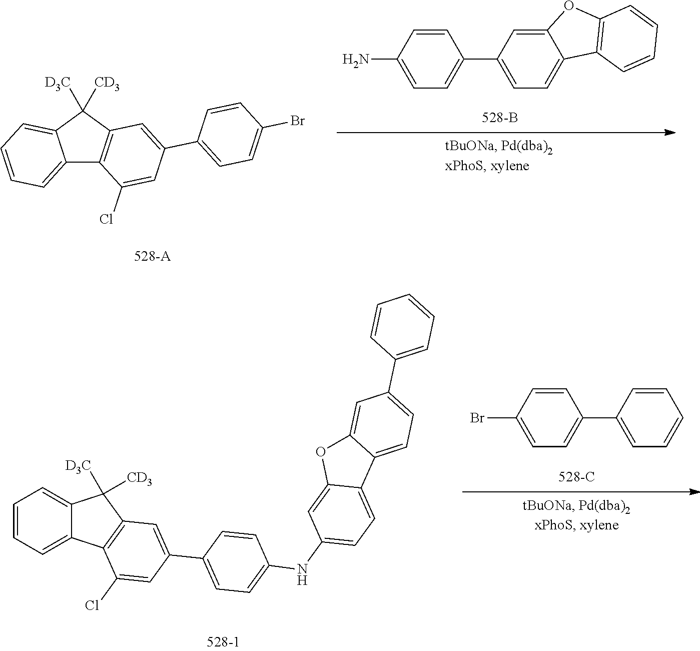 [SMILES: [2H]C([2H])([2H])C1(C([2H])([2H])[2H])c2ccccc2-c2c(Cl)cc(-c3ccc(Br)cc3)cc21 ; Nc1ccc(-c2ccc3c(c2)oc2ccccc23)cc1 ; [2H]C([2H])([2H])C1(C([2H])([2H])[2H])c2ccccc2-c2c(Cl)cc(-c3ccc(Nc4ccc5c(c4)oc4cc(-c6ccccc6)ccc45)cc3)cc21 ; Brc1ccc(-c2ccccc2)cc1]

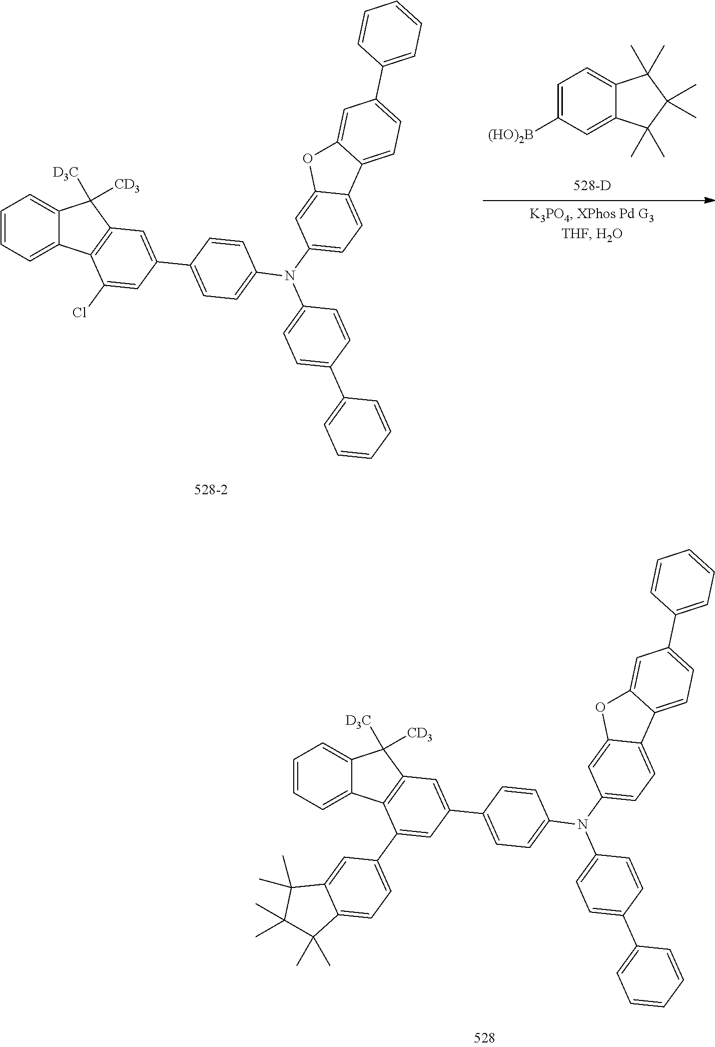 [SMILES: [2H]C([2H])([2H])C1(C([2H])([2H])[2H])c2ccccc2-c2c(Cl)cc(-c3ccc(N(c4ccc(-c5ccccc5)cc4)c4ccc5c(c4)oc4cc(-c6ccccc6)ccc45)cc3)cc21 ; CC1(C)c2ccc(B(O)O)cc2C(C)(C)C1(C)C ; [2H]C([2H])([2H])C1(C([2H])([2H])[2H])c2ccccc2-c2c(-c3ccc4c(c3)C(C)(C)C(C)(C)C4(C)C)cc(-c3ccc(N(c4ccc(-c5ccccc5)cc4)c4ccc5c(c4)oc4cc(-c6ccccc6)ccc45)cc3)cc21]

Except that the starting materials were replaced with 528-A, 528-B, 528-C and 528-D, everything else was the same as Example 88. LC MS: M/Z 885.48 (M+). Total yield of synthesis: 49%; HPLC purity: 99.9%.

¹H NMR (500 MHz, DMSO-d6) δ 7.93 (d, 1H), 7.84 (d, 1H), 7.74 (m, 3H), 7.69-7.59 (m, 4H), 7.59-7.52 (m, 5H), 7.49 (m, 1H), 7.47-7.38 (m, 3H), 7.41-7.35 (m, 5H), 7.37-7.32 (m, 1H), 7.35-7.26 (m, 2H), 7.17 (m, 6H), 7.12 (d, 1H), 7.08 (d, 1H), 6.88 (m, 1H), 1.31 (s, 12H), 0.93 (s, 6H).

### Device Example 1: Preparation of Red Light Emitting Device

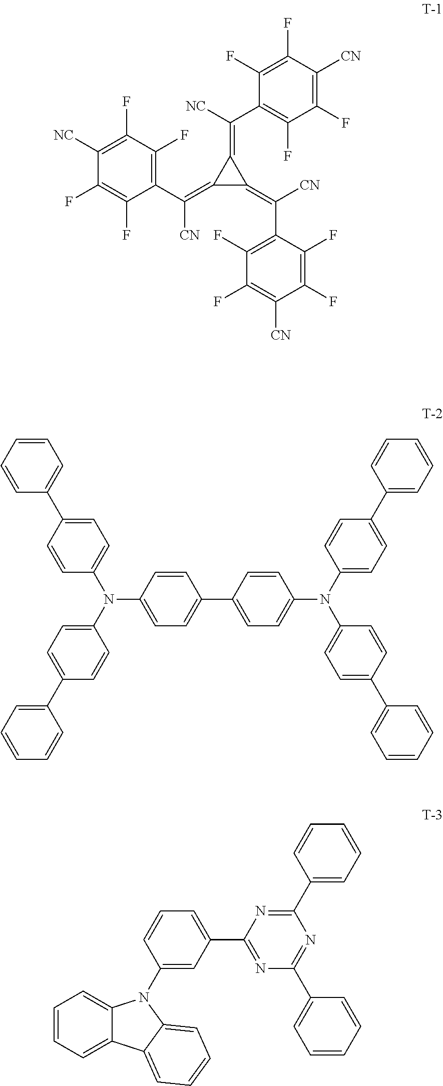 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; c1ccc(-c2nc(-c3ccccc3)nc(-c3cccc(-n4c5ccccc5c5ccccc54)c3)n2)cc1]

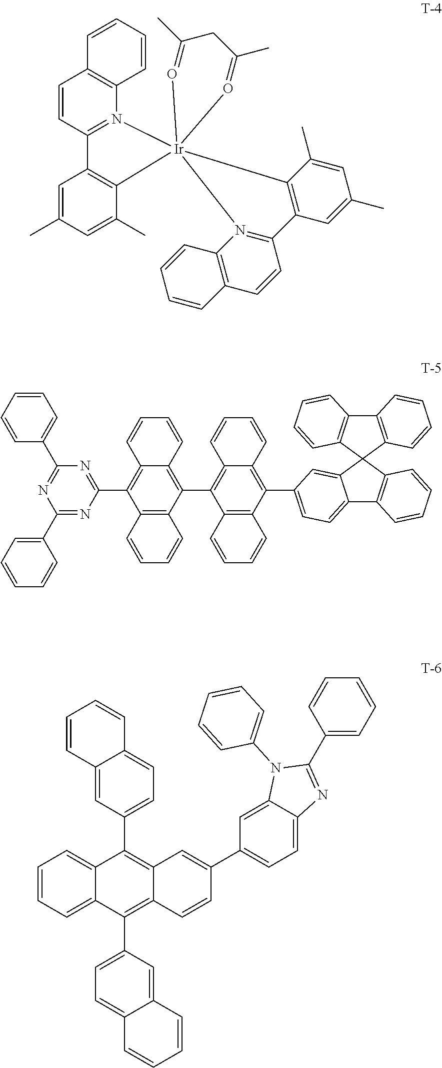

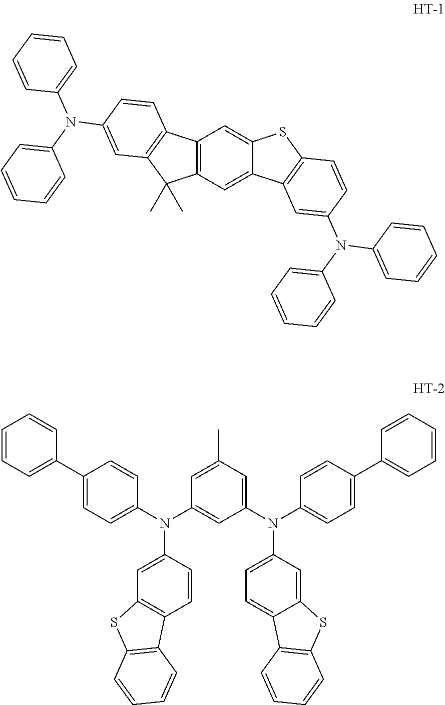 [SMILES: CC1(C)c2cc(N(c3ccccc3)c3ccccc3)ccc2-c2cc3sc4ccc(N(c5ccccc5)c5ccccc5)cc4c3cc21 ; Cc1cc(N(c2ccc(-c3ccccc3)cc2)c2ccc3c(c2)sc2ccccc23)cc(N(c2ccc(-c3ccccc3)cc2)c2ccc3c(c2)sc2ccccc23)c1]

The preparation process is as follows: a transparent anode ITO film (thickness 150 nm) was formed on a glass substrate, to obtain a first electrode as an anode. Subsequently, a mixed material of compound T-1 and compound T-2 with a mixing ratio of 3:97 (mass ratio) was deposited on the surface of the anode by vacuum evaporation, to obtain a hole injection layer with a thickness of 10 nm. Then, compound T-2 was deposited, through evaporation, on the hole injection layer to obtain a first hole transport layer with a thickness of 100 nm. Subsequently, compound 1 of the present disclosure was deposited, through evaporation, on the first hole transport layer to obtain a second hole transport layer with a thickness of 10 nm. On the second hole transport layer, compound T-3 and compound T-4 were co-deposited, through evaporation, at a mass ratio of 95:5 to form an organic light-emitting layer with a thickness of 40 nm. Then, on the organic light-emitting layer, compound T-5 was deposited, through evaporation, to form a hole blocking layer (thickness 10 nm); thereafter, compound T-6 and LiQ at a mixing ratio of 4:6 (mass ratio) were deposited, through evaporation, to form an electron transport layer (thickness 30 nm). Finally, magnesium (Mg) and silver (Ag) were mixed at a deposition rate of 1:9, and deposited, through vacuum evaporation, on the electron injection layer as the second electrode 109. As such, the organic light-emitting device was manufactured.

### Red-Light Device Examples 2-65

Except that compound 1 was replaced with compounds 20, 39, 305, 452, 457, 462, 467, 3, 28, 43, 127, 2, 11, 28, 134, 286, 6, 44, 293, 153, 172, 191, 210, 248, 267, 324, 400, 183, 204, 307, 160, 163, 170, 256, 145, 161, 175, 58, 77, 229, 57, 68, 115, 55, 64, 220, 96, 231, 100, 93, 98, 343, 381, 391, 449, 352, 363, 403, 417, 428, 419, 438, 355 and 415 respectively in forming the second hole transport layer, OLED devices were prepared by the same method as in Device Example 1.

### Red-Light Device Control Examples 1-2

Except that compound 1 was replaced with compound HT-1 and compound HT-2 respectively in forming the second hole transport layer, OLED devices were prepared in the same manner as in Device Example 1.

For the OLED devices prepared above, the operating voltage and efficiency were calculated by a Keithley 2400 test system controlled by a computer. A Polaronix (McScience Co.) lifetime measurement system, which was equipped with a power supply and a photodiode as a detection unit, was used to obtain the device lifetime under dark conditions. Each group of Red-light Device Example and Red-light Device Control Example 1 were produced and tested in the same batch as the devices of Red-light Device Control Example 2. The operating voltage, efficiency and lifetime of the devices of Red-light Device Control Example 1 are all recorded as 1, and ratios of corresponding indicators of Device Examples 1-65 and Red-light Device Control Example 2 to Red-light Device Control Example 1 were respectively calculated, as shown in Table 1.

TABLE 1
Test results of Red-light Device Examples 1-65 and Device Control Examples 1-2

| | Type of | Second hole | Relative | Relative | Relative |
|---|---|---|---|---|---|
| Example | compound | transport layer | working voltage | efficiency | lifetime |
| Red-light Device | | HT-1 | 1 | 1 | 1 |
| Control Example 1 | | | | | |
| Red-light Device | | HT-2 | 1.071 | 1.03 | 1.427 |
| Control Example 2 | | | | | |
| Red-light Device | 1 | compound 1 | 0.952 | 1.123 | 1.725 |
| Example 1 | | | | | |
| Red-light Device | 1 | compound 20 | 0.945 | 1.115 | 1.81 |
| Example 2 | | | | | |
| Red-light Device | 1 | compound 39 | 0.972 | 1.11 | 1.693 |
| Example 3 | | | | | |
| Red-light Device | 1 | compound 305 | 0.949 | 1.099 | 1.657 |
| Example 4 | | | | | |
| Red-light Device | 1 | compound 452 | 0.920 | 1.203 | 2.130 |
| Example 5 | | | | | |
| Red-light Device | 1 | compound 457 | 0.931 | 1.215 | 1.957 |
| Example 6 | | | | | |
| Red-light Device | 1 | compound 458 | 0.933 | 1.198 | 1.986 |
| Example 7 | | | | | |
| Red-light Device | 1 | compound 462 | 0.954 | 1.186 | 2.105 |
| Example 8 | | | | | |
| Red-light Device | 1 | compound 467 | 0.945 | 1.193 | 2.014 |
| Example 9 | | | | | |
| Red-light Device | 2 | compound 3 | 0.931 | 1.164 | 1.785 |
| Example 10 | | | | | |
| Red-light Device | 2 | compound 43 | 0.958 | 1.136 | 1.654 |
| Example 11 | | | | | |
| Red-light Device | 2 | compound 127 | 0.946 | 1.147 | 1.765 |
| Example 12 | | | | | |
| Red-light Device | 3 | compound 2 | 0.947 | 1.131 | 1.578 |
| Example 13 | | | | | |
| Red-light Device | 3 | compound 11 | 0.957 | 1.127 | 1.589 |
| Example 14 | | | | | |
| Red-light Device | 3 | compound 28 | 0.943 | 1.157 | 1.845 |
| Example 15 | | | | | |
| Red-light Device | 3 | compound 134 | 0.934 | 1.095 | 1.585 |
| Example 16 | | | | | |
| Red-light Device | 3 | compound 286 | 0.983 | 1.160 | 1.484 |
| Example 17 | | | | | |
| Red-light Device | 4 | compound 6 | 0.976 | 1.174 | 1.457 |
| Example 18 | | | | | |
| Red-light Device | 4 | compound 44 | 0.987 | 1.105 | 1.478 |
| Example 19 | | | | | |
| Red-light Device | 4 | compound 293 | 0.976 | 1.135 | 1.567 |
| Example 20 | | | | | |
| Red-light Device | 5 | compound 153 | 0.969 | 1.092 | 1.867 |
| Example 21 | | | | | |
| Red-light Device | 5 | compound 172 | 0.968 | 1.159 | 1.681 |
| Example 22 | | | | | |
| Red-light Device | 5 | compound 191 | 0.983 | 1.095 | 1.685 |
| Example 23 | | | | | |
| Red-light Device | 5 | compound 210 | 0.972 | 1.16 | 1.545 |
| Example 24 | | | | | |
| Red-light Device | 5 | compound 248 | 0.941 | 1.182 | 1.767 |
| Example 25 | | | | | |
| Red-light Device | 5 | compound 267 | 0.978 | 1.183 | 1.945 |
| Example 26 | | | | | |
| Red-light Device | 5 | compound 324 | 0.965 | 1.13 | 1.764 |
| Example 27 | | | | | |
| Red-light Device | 5 | compound 400 | 0.974 | 1.135 | 1.943 |
| Example 28 | | | | | |
| Red-light Device | 6 | compound 183 | 0.953 | 1.107 | 1.457 |
| Example 29 | | | | | |
| Red-light Device | 6 | compound 204 | 0.947 | 1.098 | 1.543 |
| Example 30 | | | | | |
| Red-light Device | 6 | compound 307 | 0.956 | 1.107 | 1.563 |
| Example 31 | | | | | |
| Red-light Device | 7 | compound 160 | 0.987 | 1.134 | 1.587 |
| Example 32 | | | | | |
| Red-light Device | 7 | compound 163 | 0.976 | 1.109 | 1.585 |
| Example 33 | | | | | |
| Red-light Device | 7 | compound 170 | 0.965 | 1.107 | 1.456 |
| Example 34 | | | | | |
| Red-light Device | 7 | compound 256 | 0.957 | 1.135 | 1.589 |
| Example 35 | | | | | |
| Red-light Device | 8 | compound 145 | 0.977 | 1.132 | 1.564 |
| Example 36 | | | | | |
| Red-light Device | 8 | compound 161 | 0.965 | 1.109 | 1.653 |
| Example 37 | | | | | |
| Red-light Device | 8 | compound 175 | 0.975 | 1.127 | 1.589 |
| Example 38 | | | | | |
| Red-light Device | 9 | compound 58 | 0.953 | 1.132 | 1.809 |
| Example 39 | | | | | |
| Red-light Device | 9 | compound 77 | 0.933 | 1.101 | 1.960 |
| Example 40 | | | | | |
| Red-light Device | 9 | compound 229 | 0.942 | 1.176 | 1.665 |
| Example 41 | | | | | |
| Red-light Device | 10 | compound 57 | 0.976 | 1.156 | 1.535 |
| Example 42 | | | | | |
| Red-light Device | 11 | compound 68 | 0.986 | 1.098 | 1.557 |
| Example 43 | | | | | |
| Red-light Device | 12 | compound 115 | 0.965 | 1.156 | 1.883 |
| Example 44 | | | | | |
| Red-light Device | 13 | compound 55 | 0.953 | 1.147 | 1.768 |
| Example 45 | | | | | |
| Red-light Device | 13 | compound 64 | 0.945 | 1.153 | 1.457 |
| Example 46 | | | | | |
| Red-light Device | 13 | compound 220 | 0.955 | 1.135 | 1.570 |
| Example 47 | | | | | |
| Red-light Device | 14 | compound 96 | 0.932 | 1.105 | 1.872 |
| Example 48 | | | | | |
| Red-light Device | 14 | compound 231 | 0.948 | 1.116 | 1.795 |
| Example 49 | | | | | |
| Red-light Device | 15 | compound 100 | 0.956 | 1.079 | 1.457 |
| Example 50 | | | | | |
| Red-light Device | 15 | compound 93 | 0.978 | 1.097 | 1.389 |
| Example 51 | | | | | |
| Red-light Device | 15 | compound 98 | 0.969 | 1.105 | 1.401 |
| Example 52 | | | | | |
| Red-light Device | 16 | compound 343 | 0.977 | 1.098 | 1.640 |
| Example 53 | | | | | |
| Red-light Device | 16 | compound 381 | 0.956 | 1.156 | 1.567 |
| Example 54 | | | | | |
| Red-light Device | 16 | compound 391 | 0.965 | 1.198 | 1.675 |
| Example 55 | | | | | |
| Red-light Device | 16 | compound 449 | 0.969 | 1.125 | 1.594 |
| Example 56 | | | | | |
| Red-light Device | 17 | compound 352 | 0.976 | 1.135 | 1.478 |
| Example 57 | | | | | |
| Red-light Device | 17 | compound 363 | 0.958 | 1.104 | 1.558 |
| Example 58 | | | | | |
| Red-light Device | 17 | compound 403 | 0.967 | 1.125 | 1.589 |
| Example 59 | | | | | |
| Red-light Device | 17 | compound 417 | 0.976 | 1.146 | 1.654 |
| Example 60 | | | | | |
| Red-light Device | 18 | compound 428 | 0.954 | 1.097 | 1.569 |
| Example 61 | | | | | |
| Red-light Device | 19 | compound 419 | 0.968 | 1.173 | 1.694 |
| Example 62 | | | | | |
| Red-light Device | 19 | compound 438 | 0.968 | 1.126 | 1.623 |
| Example 63 | | | | | |
| Red-light Device | 20 | compound 355 | 0.968 | 1.126 | 1.596 |
| Example 64 | | | | | |
| Red-light Device | 20 | compound 415 | 0.975 | 1.132 | 1.587 |
| Example 65 | | | | | |

According to the results in Table 1, when being used as the second hole transport layer of a red light-emitting device, the compounds used in Red-light Device Examples 1-65 enable respective formed devices to have lower voltages, higher luminous efficiencies (up to 20%), and significantly improved lifetime, compared with those formed from the compounds used in Red-light Device Control Examples 1-2. The possible reasons are as follows: compared with the compounds in the Control Examples, the introduction of a group on the side adjacent to the nitrogen atom can increase the triplet energy level of the compound; in addition, the introduced group may form a weak conjugation with the nitrogen atom, so that the material is more stable. Furthermore, from the aforementioned types of compounds, the efficiency and lifetime of devices made of these types of materials are greatly improved compared with the reference compounds, and there is not much difference between these types of materials in red light devices. Therefore, the compounds of the present disclosure are all suitable for red light devices.

### Green-Light Device Example 1

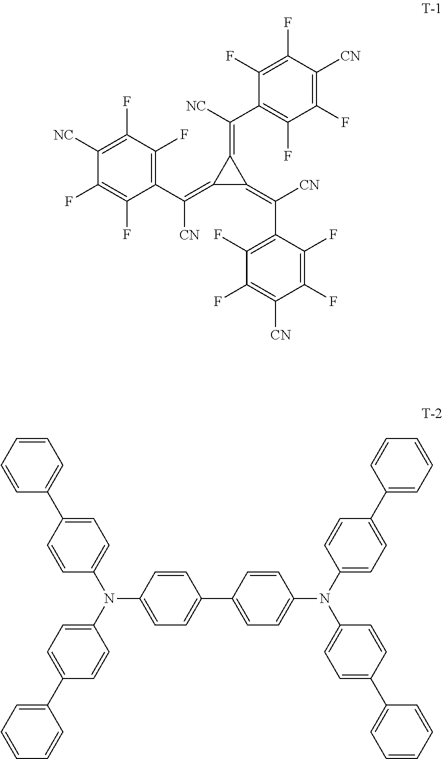 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1]

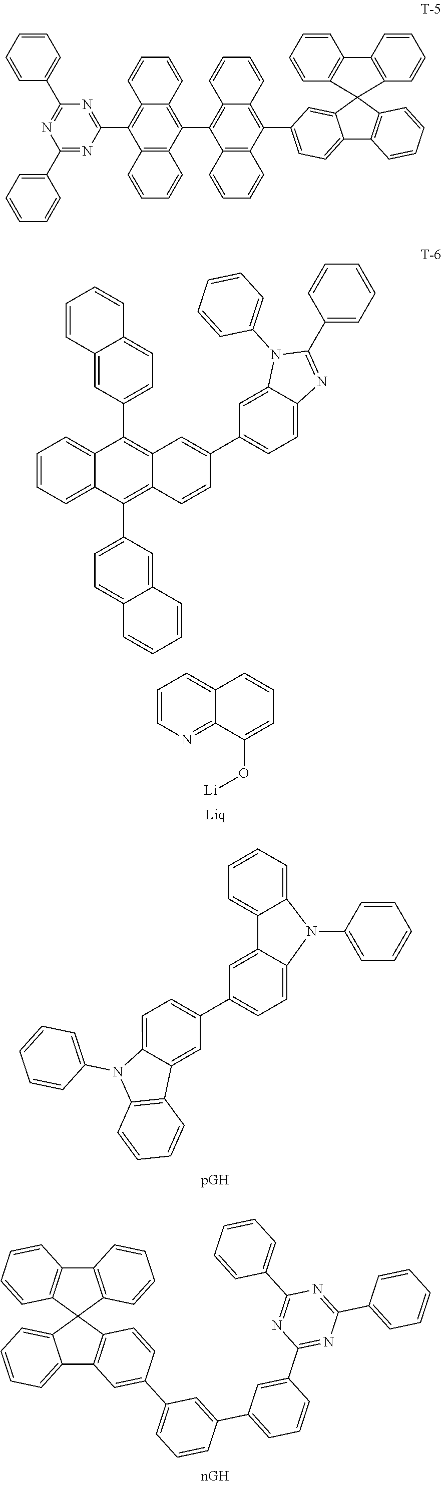

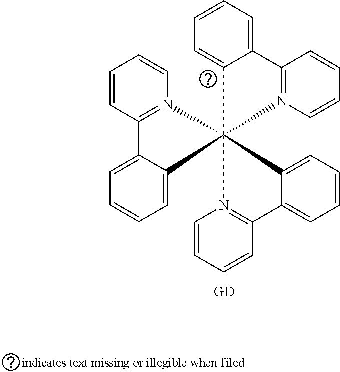

The preparation process is as follows: on a glass substrate, a transparent ITO film (thickness 150 nm) was formed by a magnetron sputtering process, to obtain a first electrode as an anode. A mixed material of compound T-1 and compound T-2 was deposited, through evaporation, on the surface of the anode as a hole injection layer. Then, T-2 (thickness 100 nm) and compound 1 of the present disclosure (thickness 40 nm) were deposited thereon to obtain a first hole transport layer and a second hole transport layer, respectively. Next, on the surface of the second hole transport layer, compound pGH, compound nGH and compound GD were co-deposited, through evaporation, at a mass ratio of 45:45:10 to form an organic light-emitting layer (thickness 40 nm). Subsequently, compound T-5 was deposited, through evaporation, on the surface of the organic light-emitting layer to form a hole blocking layer (10 nm thick); thereafter, compound T-6 and LiQ at a mixing ratio of 4:6 (mass ratio) were formed as an electron transport layer (30 nm thick). Finally, magnesium (Mg) and silver (Ag) were mixed and deposited at an evaporation rate of 1:9 on the surface of the electron transport layer, to form a second electrode with a thickness of 10 nm as a cathode. As such, the organic light-emitting device was manufactured.

### Green-Light Device Examples 2-51

Except that compound 1 was replaced with compounds 4, 20, 30, 38, 452, 456, 457, 458, 461, 462, 464, 465, 467, 43, 127, 2, 11, 6, 293, 153, 191, 248, 400, 204, 307, 163, 256, 145, 175, 58, 77, 57, 68, 115, 55, 220, 96, 229, 100, 93, 98, 343, 391, 403,417, 428, 419, 438, 439 and 440 respectively in forming the second hole transport layer, OLED devices were prepared by the same method as in Green-light Device Example 1.

### Green-Light Device Control Examples 1-4

Except that compound 1 was replaced with compounds HT-3, HT-4, HT-5 and HT-6 in forming the second hole transport layer, OLED devices were prepared by the same method as in Green-light Device Example 1.

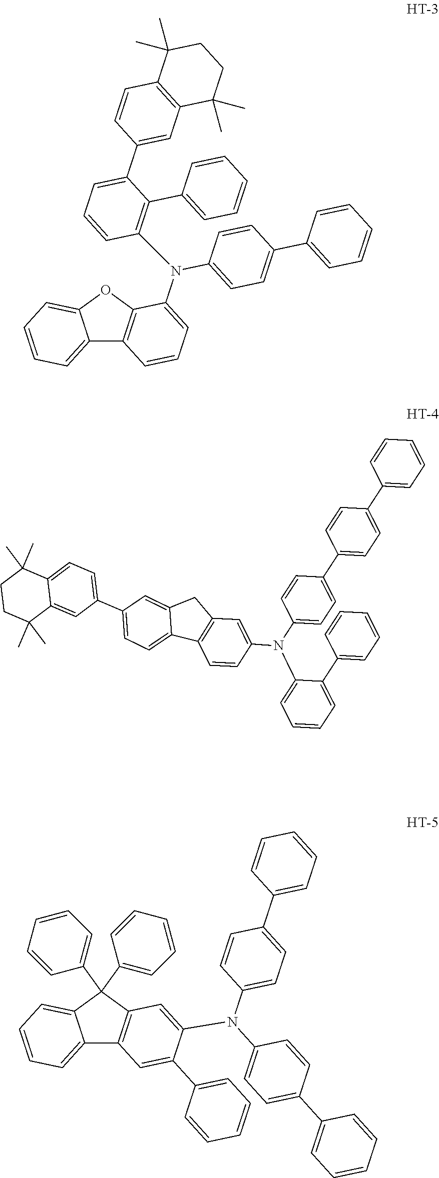 [SMILES: CC1(C)CCC(C)(C)c2cc(-c3cccc(N(c4ccc(-c5ccccc5)cc4)c4cccc5c4oc4ccccc45)c3-c3ccccc3)ccc21 ; CC1(C)CCC(C)(C)c2cc(-c3ccc4c(c3)Cc3cc(N(c5ccc(-c6ccc(-c7ccccc7)cc6)cc5)c5ccccc5-c5ccccc5)ccc3-4)ccc21 ; c1ccc(-c2ccc(N(c3ccc(-c4ccccc4)cc3)c3cc4c(cc3-c3ccccc3)-c3ccccc3C4(c3ccccc3)c3ccccc3)cc2)cc1]

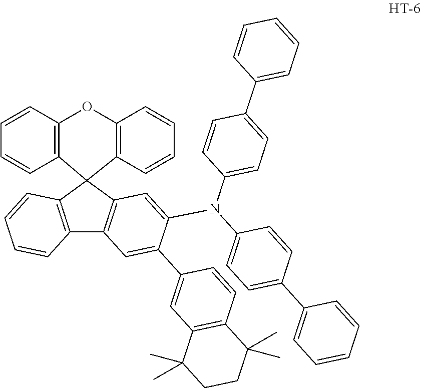 [SMILES: CC1(C)CCC(C)(C)c2cc(-c3cc4c(cc3N(c3ccc(-c5ccccc5)cc3)c3ccc(-c5ccccc5)cc3)C3(c5ccccc5Oc5ccccc53)c3ccccc3-4)ccc21]

TABLE 2
Test results of Green-light Device Examples 1-51
and Green-light Device Control Examples 1-4

| | Type of | Second hole | Relative | Relative | Relative |
|---|---|---|---|---|---|
| Example | compound | transport layer | working voltage | efficiency | lifetime |
| Green-light Device | | HT-3 | 1 | 1 | 1 |
| Control Example 1 | | | | | |
| Green-light Device | | HT-4 | 1.083 | 1.03 | 1.403 |
| Control Example 2 | | | | | |
| Green-light Device | | HT-5 | 1.108 | 0.996 | 1.536 |
| Control Example 3 | | | | | |
| Green-light Device | | HT-6 | 1.054 | 0.986 | 1.389 |
| Control Example 4 | | | | | |
| Green-light Device | 1 | compound 1 | 0.955 | 1.126 | 1.870 |
| Example 1 | | | | | |
| Green-light Device | 1 | compound 4 | 0.963 | 1.127 | 1.863 |
| Example 2 | | | | | |
| Green-light Device | 1 | compound 20 | 0.967 | 1.132 | 1.869 |
| Example 3 | | | | | |
| Green-light Device | 1 | compound 30 | 0.976 | 1.131 | 1.860 |
| Example 4 | | | | | |
| Green-light Device | 1 | compound 38 | 0.942 | 1.145 | 1.972 |
| Example 5 | | | | | |
| Green-light Device | 1 | compound 452 | 0.965 | 1.184 | 2.131 |
| Example 6 | | | | | |
| Green-light Device | 1 | compound 456 | 0.943 | 1.161 | 1.985 |
| Example 7 | | | | | |
| Green-light Device | 1 | compound 457 | 0.95 | 1.152 | 1.967 |
| Example 8 | | | | | |
| Green-light Device | 1 | compound 458 | 0.957 | 1.156 | 1.981 |
| Example 9 | | | | | |
| Green-light Device | 1 | compound 461 | 0.933 | 1.145 | 1.931 |
| Example 10 | | | | | |
| Green-light Device | 1 | compound 462 | 0.975 | 1.159 | 1.956 |
| Example 11 | | | | | |
| Green-light Device | 1 | compound 464 | 0.937 | 1.169 | 1.939 |
| Example 12 | | | | | |
| Green-light Device | 1 | compound 465 | 0.941 | 1.171 | 1.917 |
| Example 13 | | | | | |
| Green-light Device | 1 | compound 467 | 0.978 | 1.162 | 1.934 |
| Example 14 | | | | | |
| Green-light Device | 2 | compound 43 | 1.043 | 1.067 | 1.561 |
| Example 15 | | | | | |
| Green-light Device | 2 | compound 127 | 0.987 | 1.063 | 1.665 |
| Example 16 | | | | | |
| Green-light Device | 3 | compound 2 | 0.998 | 1.100 | 1.602 |
| Example 17 | | | | | |
| Green-light Device | 3 | compound 11 | 0.999 | 1.099 | 1.574 |
| Example 18 | | | | | |
| Green-light Device | 4 | compound 6 | 0.987 | 1.080 | 1.583 |
| Example 19 | | | | | |
| Green-light Device | 4 | compound 293 | 1.039 | 1.053 | 1.592 |
| Example 20 | | | | | |
| Green-light Device | 5 | compound 153 | 0.981 | 1.074 | 1.553 |
| Example 21 | | | | | |
| Green-light Device | 5 | compound 191 | 0.983 | 1.062 | 1.559 |
| Example 22 | | | | | |
| Green-light Device | 5 | compound 248 | 1.050 | 1.050 | 1.641 |
| Example 23 | | | | | |
| Green-light Device | 5 | compound 400 | 1.035 | 1.050 | 1.584 |
| Example 24 | | | | | |
| Green-light Device | 6 | compound 204 | 1.048 | 1.097 | 1.662 |
| Example 25 | | | | | |
| Green-light Device | 6 | compound 307 | 1.044 | 1.092 | 1.618 |
| Example 26 | | | | | |
| Green-light Device | 7 | compound 163 | 0.996 | 1.094 | 1.693 |
| Example 27 | | | | | |
| Green-light Device | 7 | compound 256 | 1.005 | 1.046 | 1.681 |
| Example 28 | | | | | |
| Green-light Device | 8 | compound 145 | 0.997 | 1.071 | 1.543 |
| Example 29 | | | | | |
| Green-light Device | 8 | compound 175 | 0.994 | 1.060 | 1.564 |
| Example 30 | | | | | |
| Green-light Device | 9 | compound 58 | 1.002 | 1.040 | 1.638 |
| Example 31 | | | | | |
| Green-light Device | 9 | compound 77 | 1.047 | 1.050 | 1.587 |
| Example 32 | | | | | |
| Green-light Device | 10 | compound 57 | 1.014 | 1.083 | 1.663 |
| Example 33 | | | | | |
| Green-light Device | 11 | compound 68 | 1.048 | 1.068 | 1.677 |
| Example 34 | | | | | |
| Green-light Device | 12 | compound 115 | 1.039 | 1.077 | 1.665 |
| Example 35 | | | | | |
| Green-light Device | 13 | compound 55 | 1.029 | 1.055 | 1.671 |
| Example 36 | | | | | |
| Green-light Device | 13 | compound 220 | 1.014 | 1.051 | 1.577 |
| Example 37 | | | | | |
| Green-light Device | 14 | compound 96 | 1.042 | 1.085 | 1.640 |
| Example 38 | | | | | |
| Green-light Device | 14 | compound 231 | 1.044 | 1.083 | 1.556 |
| Example 39 | | | | | |
| Green-light Device | 15 | compound 100 | 1.004 | 1.077 | 1.605 |
| Example 40 | | | | | |
| Green-light Device | 15 | compound 93 | 1.046 | 1.059 | 1.555 |
| Example 41 | | | | | |
| Green-light Device | 15 | compound 98 | 1.008 | 1.063 | 1.599 |
| Example 42 | | | | | |
| Green-light Device | 16 | compound 343 | 1.041 | 1.043 | 1.669 |
| Example 43 | | | | | |
| Green-light Device | 16 | compound 391 | 1.012 | 1.057 | 1.683 |
| Example 44 | | | | | |
| Green-light Device | 17 | compound 403 | 1.008 | 1.095 | 1.598 |
| Example 45 | | | | | |
| Green-light Device | 17 | compound 417 | 0.984 | 1.073 | 1.613 |
| Example 46 | | | | | |
| Green-light Device | 18 | compound 428 | 1.017 | 1.041 | 1.543 |
| Example 47 | | | | | |
| Green-light Device | 19 | compound 419 | 0.983 | 1.065 | 1.554 |
| Example 48 | | | | | |
| Green-light Device | 19 | compound 438 | 1.037 | 1.048 | 1.545 |
| Example 49 | | | | | |
| Green-light Device | 20 | compound 355 | 0.982 | 1.056 | 1.665 |
| Example 50 | | | | | |
| Green-light Device | 20 | compound 415 | 1.030 | 1.082 | 1.549 |
| Example 51 | | | | | |

According to the results in Table 2, when being used as the second hole transport layer of a green light-emitting device, the compounds used in Green-light Device Examples 1 to 51 enable respective formed devices to have lower voltages, higher luminous efficiencies, and significantly improved lifetime, compared with those formed from compounds used in Green-light Device Control Examples 1 to 4. In addition, Green-light Device Examples 15 to 51 (with heteroatoms in A ring or B ring) have lower efficiency and shorter lifetime than those of Green-light Device Examples 1 to 14 (without heteroatoms in A ring or B ring). However, when the compounds in Examples 15 to 51 were used in red light devices, they had the same gain effect as the compounds without heteroatoms in A ring and B ring. The reason may be that, in green light devices, the energy of excitons is higher, which requires higher thermal and electrical stability of the materials. There are some unstable sites (such as some heteroatoms) in these compounds, which leads to a decrease in the efficiency and lifetime of the device. In addition, in Example 6, deuterium atoms were introduced into compound 452, which improved the lifetime of the device.

In summary, regardless of whether A ring or B ring of the compound of the present disclosure contains heteroatoms, when such compound is applied to a red light device, the voltage of the device is significantly reduced, and the efficiency and lifetime of the device are greatly improved. When A ring or B ring contains heteroatoms, such compound is not suitable for application in a green light device. When A ring or B ring does not contain heteroatoms, the voltage of the green light device is significantly reduced, and the efficiency and lifetime of the green light device are greatly improved, which is more suitable for application in green light devices.

### Blue-Light Device Example 1: Preparation of OLED Device

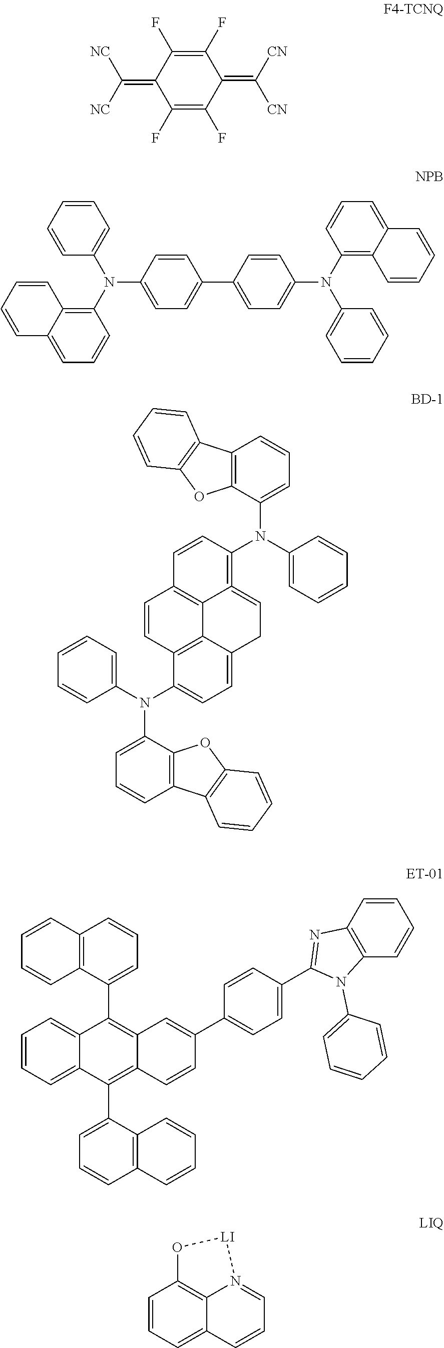

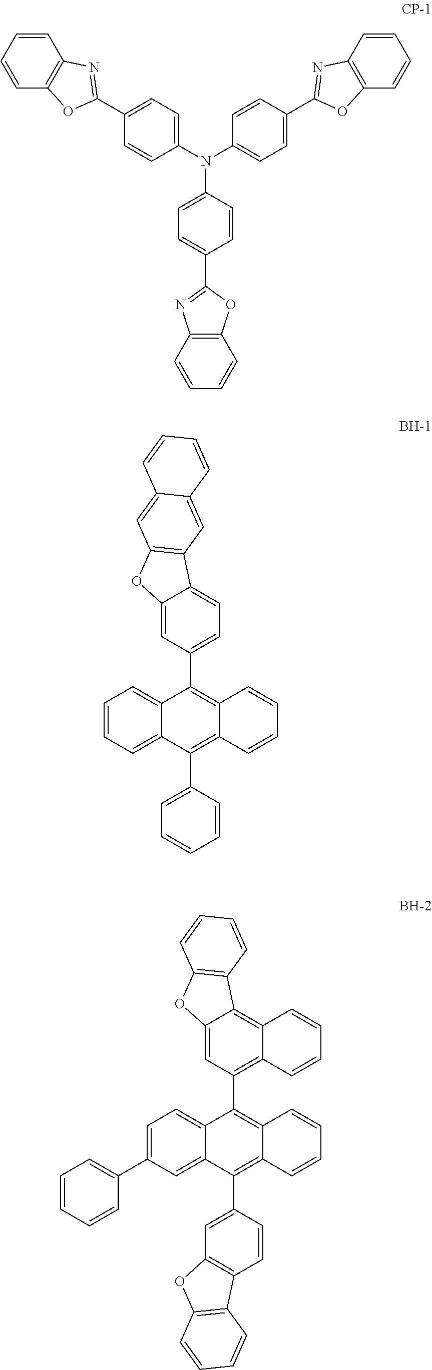 [SMILES: c1ccc2oc(-c3ccc(N(c4ccc(-c5nc6ccccc6o5)cc4)c4ccc(-c5nc6ccccc6o5)cc4)cc3)nc2c1 ; c1ccc(-c2c3ccccc3c(-c3ccc4c(c3)oc3cc5ccccc5cc34)c3ccccc23)cc1 ; c1ccc(-c2ccc3c(-c4cc5oc6ccccc6c5c5ccccc45)c4ccccc4c(-c4ccc5c(c4)oc4ccccc45)c3c2)cc1]

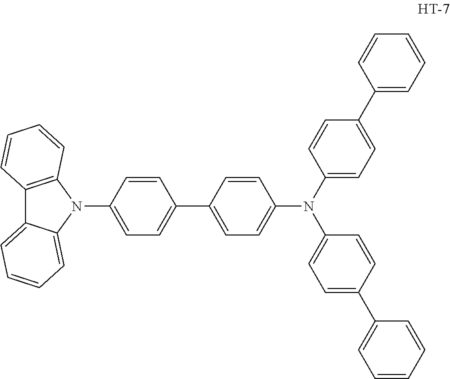 [SMILES: c1ccc(-c2ccc(N(c3ccc(-c4ccccc4)cc3)c3ccc(-c4ccc(-n5c6ccccc6c6ccccc65)cc4)cc3)cc2)cc1]

The preparation process is as follows:

1) A transparent anode ITO film (thickness 150 nm) was formed on a glass substrate, to obtain a first electrode as an anode.

2) Compound F4-TCNQ was deposited, through vacuum evaporation, on the surface of the anode, to form a hole injection layer with a thickness of 10 nm. Compound NPB (thickness 100 nm) and compound 1 (40 nm) were deposited, through vacuum evaporation, on the hole injection layer, to form a first hole transport layer and a second hole transport layer, respectively.

4) On the surface of the second hole transport layer, an emission layer (EML) with a thickness of 10 nm was formed by taking compound BH-1 as a main component and doping BD-1 at a film thickness ratio of 100:3.

5) ET-01 and LiQ were deposited, through evaporation, at a film thickness ratio of 1:1 on the EML, to form an electron transport layer (ETL) with a thickness of 30 nm, and Yb was deposited, through evaporation, on the electron transport layer to form an electron injection layer (EIL) with a thickness of 15 angstroms.

6) Magnesium (Mg) and silver (Ag) were deposited, through vacuum evaporation, at a film thickness ratio of 1:9 on the electron injection layer, to form a cathode with a thickness of 11 nm.

7) CP-1 with a thickness of 65 nm was deposited, through evaporation, on the cathode, to serve as an organic cover layer (CPL). As such, the organic light-emitting device was manufactured.

### Blue-Light Device Examples 2-105

Except that compound 1 was replaced with compounds 4, 20, 30, 38, 39, 305, 457, 458, 462, 463, 464, 465, 466, 467, 479, 480, 485, 494, 504, 507, 509, 514, 519, 521, 524, 526, 527, 528, 3, 28, 43, 127, 2, 11, 134, 286, 478, 481, 493, 6, 44, 293, 153, 172, 191, 210, 248, 267, 324, 400, 486, 487, 488, 489, 491, 492, 495, 183, 204, 307, 160, 163, 170, 256, 145, 161, 175, 58, 77, 482, 490, 499, 57, 68, 115, 55, 64, 220, 484, 96, 229, 483, 100, 93, 98, 496, 497, 498, 500, 501, 502, 343, 381, 391, 449, 352, 363, 403, 417, 428, 419, 438, 355 and 439 respectively in forming the second hole transport layer, OLED devices were prepared by the same method as in Blue-light Device Example 1.

### Blue-Light Device Control Examples 1-7

Except that compound 1 was replaced with compound HT-1, HT-2, HT-3, HT-4, HT-5, HT-6 and compound HT-7 respectively in forming the second hole transport layer, OLED devices were prepared by the same method as in Blue-light Device Example 1.

Each of the above Device Examples and Device Control Examples were produced and tested in the same batch as the devices of Device Control Example 1. The operating voltage, efficiency and lifetime of the device of Device Control Example 1 are all recorded as 1, and the ratios of corresponding indicators of Device Examples 1-105 and Device Control Examples 2-7 to Device Control Example 1 were respectively calculated, as shown in Table 3.

TABLE 3
Test results of Blue-light Device Examples 1-105 and Device Control Examples 1-7

| | Type of | Second hole | Relative | Relative | Relative |
|---|---|---|---|---|---|
| Example | compound | transport layer | working voltage | efficiency | lifetime |
| Blue-light Device | | HT-1 | 1 | 1 | 1 |
| Control Examples 1 | | | | | |
| Blue-light Device | | HT-2 | 1.135 | 0.976 | 0.90 |
| Control Examples 2 | | | | | |
| Blue-light Device | | HT-3 | 0.986 | 1.035 | 1.210 |
| Control Examples 3 | | | | | |
| Blue-light Device | | HT-4 | 1.036 | 1.010 | 0.965 |
| Control Examples 4 | | | | | |
| Blue-light Device | | HT-5 | 1.231 | 0.976 | 1.015 |
| Control Examples 5 | | | | | |
| Blue-light Device | | HT-6 | 1.136 | 1.113 | 1.214 |
| Control Examples 6 | | | | | |
| Blue-light Device | | HT-7 | 1.158 | 1.096 | 1.104 |
| Control Examples 7 | | | | | |
| Blue-light Device | 1 | compound 1 | 0.940 | 1.210 | 1.675 |
| Example 1 | | | | | |
| Blue-light Device | 1 | compound 4 | 0.956 | 1.175 | 1.535 |
| Example 2 | | | | | |
| Blue-light Device | 1 | compound 20 | 0.961 | 1.136 | 1.765 |
| Example 3 | | | | | |
| Blue-light Device | 1 | compound 30 | 0.953 | 1.158 | 1.679 |
| Example 4 | | | | | |
| Blue-light Device | 1 | compound 38 | 0.972 | 1.196 | 1.456 |
| Example 5 | | | | | |
| Blue-light Device | 1 | compound 39 | 0.988 | 1.147 | 1.765 |
| Example 6 | | | | | |
| Blue-light Device | 1 | compound 305 | 0.961 | 1.157 | 1.765 |
| Example 7 | | | | | |
| Blue-light Device | 1 | compound 457 | 0.945 | 1.196 | 1.887 |
| Example 8 | | | | | |
| Blue-light Device | 1 | compound 458 | 0.940 | 1.176 | 1.901 |
| Example 9 | | | | | |
| Blue-light Device | 1 | compound 462 | 0.946 | 1.187 | 1.915 |
| Example 10 | | | | | |
| Blue-light Device | 1 | compound 463 | 0.957 | 1.189 | 1.898 |
| Example 11 | | | | | |
| Blue-light Device | 1 | compound 464 | 0.965 | 1.175 | 1.932 |
| Example 12 | | | | | |
| Blue-light Device | 1 | compound 465 | 0.945 | 1.183 | 1.967 |
| Example 13 | | | | | |
| Blue-light Device | 1 | compound 466 | 0.963 | 1.196 | 1.967 |
| Example 14 | | | | | |
| Blue-light Device | 1 | compound 467 | 0.975 | 1.173 | 1.898 |
| Example 15 | | | | | |
| Blue-light Device | 1 | compound 479 | 0.965 | 1.298 | 1.978 |
| Example 16 | | | | | |
| Blue-light Device | 1 | compound 480 | 0.956 | 1.287 | 2.136 |
| Example 17 | | | | | |
| Blue-light Device | 1 | compound 485 | 0.969 | 1.295 | 2.108 |
| Example 18 | | | | | |
| Blue-light Device | 1 | compound 494 | 0.943 | 1.335 | 2.103 |
| Example 19 | | | | | |
| Blue-light Device | 1 | compound 504 | 0.936 | 1.361 | 2.315 |
| Example 20 | | | | | |
| Blue-light Device | 1 | compound 507 | 0.948 | 1.313 | 1.993 |
| Example 21 | | | | | |
| Blue-light Device | 1 | compound 509 | 0.958 | 1.306 | 2.008 |
| Example 22 | | | | | |
| Blue-light Device | 1 | compound 514 | 0.967 | 1.296 | 2.189 |
| Example 23 | | | | | |
| Blue-light Device | 1 | compound 519 | 0.976 | 1.231 | 1.989 |
| Example 24 | | | | | |
| Blue-light Device | 1 | compound 521 | 0.958 | 1.203 | 2.031 |
| Example 25 | | | | | |
| Blue-light Device | 1 | compound 524 | 0.960 | 1.298 | 2.065 |
| Example 26 | | | | | |
| Blue-light Device | 1 | compound 526 | 0.953 | 1.207 | 2.187 |
| Example 27 | | | | | |
| Blue-light Device | 1 | compound 527 | 0.967 | 1.298 | 2.201 |
| Example 28 | | | | | |
| Blue-light Device | 1 | compound 528 | 0.971 | 1.306 | 2.109 |
| Example 29 | | | | | |
| Blue-light Device | 2 | compound 3 | 0.978 | 1.153 | 1.767 |
| Example 30 | | | | | |
| Blue-light Device | 2 | compound 28 | 0.965 | 1.176 | 1.891 |
| Example 31 | | | | | |
| Blue-light Device | 2 | compound 43 | 0.957 | 1.189 | 1.764 |
| Example 32 | | | | | |
| Blue-light Device | 2 | compound 127 | 0.952 | 1.177 | 1.976 |
| Example 33 | | | | | |
| Blue-light Device | 3 | compound 2 | 0.968 | 1.169 | 1.825 |
| Example 34 | | | | | |
| Blue-light Device | 3 | compound 11 | 0.974 | 1.175 | 1.919 |
| Example 35 | | | | | |
| Blue-light Device | 3 | compound 134 | 0.965 | 1.168 | 1.869 |
| Example 36 | | | | | |
| Blue-light Device | 3 | compound 286 | 0.967 | 1.181 | 1.686 |
| Example 37 | | | | | |
| Blue-light Device | 3 | compound 478 | 0.954 | 1.205 | 1.992 |
| Example 38 | | | | | |
| Blue-light Device | 3 | compound 481 | 0.971 | 1.193 | 2.015 |
| Example 39 | | | | | |
| Blue-light Device | 3 | compound 493 | 0.965 | 1.216 | 1.989 |
| Example 40 | | | | | |
| Blue-light Device | 4 | compound 6 | 0.976 | 1.178 | 1.654 |
| Example 41 | | | | | |
| Blue-light Device | 4 | compound 44 | 0.979 | 1.168 | 1.587 |
| Example 42 | | | | | |
| Blue-light Device | 4 | compound 293 | 0.953 | 1.186 | 1.596 |
| Example 43 | | | | | |
| Blue-light Device | 5 | compound 153 | 0.971 | 1.195 | 1.865 |
| Example 44 | | | | | |
| Blue-light Device | 5 | compound 172 | 0.963 | 1.186 | 1.690 |
| Example 45 | | | | | |
| Blue-light Device | 5 | compound 191 | 0.954 | 1.178 | 1.774 |
| Example 46 | | | | | |
| Blue-light Device | 5 | compound 210 | 0.976 | 1.157 | 1.636 |
| Example 47 | | | | | |
| Blue-light Device | 5 | compound 248 | 0.958 | 1.196 | 1.858 |
| Example 48 | | | | | |
| Blue-light Device | 5 | compound 267 | 0.969 | 1.185 | 1.967 |
| Example 49 | | | | | |
| Blue-light Device | 5 | compound 324 | 0.971 | 1.174 | 1.853 |
| Example 50 | | | | | |
| Blue-light Device | 5 | compound 400 | 0.979 | 1.176 | 1.941 |
| Example 51 | | | | | |
| Blue-light Device | 5 | compound 486 | 0.981 | 1.218 | 1.995 |
| Example 52 | | | | | |
| Blue-light Device | 5 | compound 487 | 0.954 | 1.225 | 1.967 |
| Example 53 | | | | | |
| Blue-light Device | 5 | compound 488 | 0.965 | 1.219 | 2.034 |
| Example 54 | | | | | |
| Blue-light Device | 5 | compound 489 | 0.968 | 1.237 | 2.056 |
| Example 55 | | | | | |
| Blue-light Device | 5 | compound 491 | 0.972 | 1.219 | 2.135 |
| Example 56 | | | | | |
| Blue-light Device | 5 | compound 492 | 0.963 | 1.231 | 2.178 |
| Example 57 | | | | | |
| Blue-light Device | 5 | compound 495 | 0.957 | 1.220 | 2.164 |
| Example 58 | | | | | |
| Blue-light Device | 6 | compound 183 | 0.963 | 1.161 | 1.587 |
| Example 59 | | | | | |
| Blue-light Device | 6 | compound 204 | 0.949 | 1.176 | 1.654 |
| Example 60 | | | | | |
| Blue-light Device | 6 | compound 307 | 0.956 | 1.158 | 1.674 |
| Example 61 | | | | | |
| Blue-light Device | 7 | compound 160 | 0.977 | 1.161 | 1.696 |
| Example 62 | | | | | |
| Blue-light Device | 7 | compound 163 | 0.968 | 1.175 | 1.671 |
| Example 63 | | | | | |
| Blue-light Device | 7 | compound 170 | 0.955 | 1.169 | 1.581 |
| Example 64 | | | | | |
| Blue-light Device | 7 | compound 256 | 0.968 | 1.186 | 1.678 |
| Example 65 | | | | | |
| Blue-light Device | 8 | compound 145 | 0.965 | 1.174 | 1.675 |
| Example 66 | | | | | |
| Blue-light Device | 8 | compound 161 | 0.954 | 1.165 | 1.742 |
| Example 67 | | | | | |
| Blue-light Device | 8 | compound 175 | 0.966 | 1.173 | 1.698 |
| Example 68 | | | | | |
| Blue-light Device | 9 | compound 58 | 0.962 | 1.181 | 1.910 |
| Example 69 | | | | | |
| Blue-light Device | 9 | compound 77 | 0.951 | 1.169 | 1.871 |
| Example 70 | | | | | |
| Blue-light Device | 9 | compound 482 | 0.963 | 1.198 | 1.816 |
| Example 71 | | | | | |
| Blue-light Device | 9 | compound 490 | 0.958 | 1.201 | 2.103 |
| Example 72 | | | | | |
| Blue-light Device | 9 | compound 499 | 0.967 | 1.213 | 2.098 |
| Example 73 | | | | | |
| Blue-light Device | 10 | compound 57 | 0.958 | 1.164 | 1.657 |
| Example 74 | | | | | |
| Blue-light Device | 11 | compound 68 | 0.967 | 1.175 | 1.668 |
| Example 75 | | | | | |
| Blue-light Device | 12 | compound 115 | 0.956 | 1.168 | 1.992 |
| Example 76 | | | | | |
| Blue-light Device | 13 | compound 55 | 0.957 | 1.158 | 1.657 |
| Example 77 | | | | | |
| Blue-light Device | 13 | compound 64 | 0.958 | 1.164 | 1.566 |
| Example 78 | | | | | |
| Blue-light Device | 13 | compound 220 | 0.961 | 1.167 | 1.681 |
| Example 79 | | | | | |
| Blue-light Device | 13 | compound 484 | 0.974 | 1.215 | 1.986 |
| Example 80 | | | | | |
| Blue-light Device | 14 | compound 96 | 0.967 | 1.105 | 1.863 |
| Example 81 | | | | | |
| Blue-light Device | 14 | compound 229 | 0.958 | 1.178 | 1.574 |
| Example 82 | | | | | |
| Blue-light Device | 14 | compound 483 | 0.967 | 1.223 | 2.015 |
| Example 83 | | | | | |
| Blue-light Device | 15 | compound 100 | 0.969 | 1.159 | 1.679 |
| Example 84 | | | | | |
| Blue-light Device | 16 | compound 93 | 0.971 | 1.176 | 1.769 |
| Example 85 | | | | | |
| Blue-light Device | 16 | compound 98 | 0.972 | 1.195 | 1.890 |
| Example 86 | | | | | |
| Blue-light Device | 16 | compound 496 | 0.961 | 1.236 | 2.105 |
| Example 87 | | | | | |
| Blue-light Device | 16 | compound 497 | 0.958 | 1.219 | 2.076 |
| Example 88 | | | | | |
| Blue-light Device | 16 | compound 498 | 0.956 | 1.238 | 2.010 |
| Example 89 | | | | | |
| Blue-light Device | 16 | compound 500 | 0.967 | 1.215 | 2.045 |
| Example 90 | | | | | |
| Blue-light Device | 16 | compound 501 | 0.965 | 1.209 | 2.145 |
| Example 91 | | | | | |
| Blue-light Device | 16 | compound 502 | 0.957 | 1.217 | 2.236 |
| Example 92 | | | | | |
| Blue-light Device | 17 | compound 343 | 0.970 | 1.165 | 1.640 |
| Example 93 | | | | | |
| Blue-light Device | 17 | compound 381 | 0.964 | 1.158 | 1.567 |
| Example 94 | | | | | |
| Blue-light Device | 17 | compound 391 | 0.955 | 1.184 | 1.675 |
| Example 95 | | | | | |
| Blue-light Device | 17 | compound 449 | 0.958 | 1.156 | 1.594 |
| Example 96 | | | | | |
| Blue-light Device | 18 | compound 352 | 0.967 | 1.167 | 1.478 |
| Example 97 | | | | | |
| Blue-light Device | 18 | compound 363 | 0.969 | 1.176 | 1.558 |
| Example 98 | | | | | |
| Blue-light Device | 18 | compound 403 | 0.956 | 1.185 | 1.589 |
| Example 99 | | | | | |
| Blue-light Device | 18 | compound 417 | 0.965 | 1.176 | 1.654 |
| Example 100 | | | | | |
| Blue-light Device | 19 | compound 428 | 0.963 | 1.167 | 1.569 |
| Example 101 | | | | | |
| Blue-light Device | 20 | compound 419 | 0.959 | 1.184 | 1.694 |
| Example 102 | | | | | |
| Blue-light Device | 20 | compound 438 | 0.949 | 1.198 | 1.587 |
| Example 103 | | | | | |
| Blue-light Device | 21 | compound 355 | 0.957 | 1.176 | 1.587 |
| Example 104 | | | | | |
| Blue-light Device | 21 | compound 439 | 0.974 | 1.189 | 1.587 |
| Example 105 | | | | | |

According to the results in Table 3, when being used as the second hole transport layer of a blue light-emitting device, the compounds used in Blue-light Device Examples 1 to 105 enable respective formed devices to have lower voltages, higher luminous efficiencies, and significantly improved lifetime, compared with those formed from the compounds used in Blue-light Device Control Examples 1 to 7. Moreover, the efficiencies and lifetime of Blue-light Device Examples 16-29, Examples 38-40, Examples 52-58, Examples 71-73, Example 80, Example 83, and Examples 87-92 (introducing a benzene ring between the N atom and the matrix of an aromatic alkyl substituent) are greatly improved compared with other Examples. The possible reason is that, after the introduction of the benzene ring, the conjugation of the molecules is increased, and the degree of electron delocalization is increased, which leads to a decrease in the triplet energy level. As a result, in blue light devices, the efficiency and lifetime of the material can be greatly improved.

Therefore, the compounds of the present disclosure has great application value in organic optoelectronic devices.

The foregoing is only preferred specific implementations of the present disclosure, but the protection scope of the present disclosure is not limited thereto. Any skilled in the art can make equivalent replacements or variants according to the technical scheme and inventive concept of the present disclosure within the technical scope disclosed by the present disclosure, which should be covered by the protection scope of the present disclosure.

What is claimed is:

1. A compound, wherein a chemical structure of the compound is shown in formula (II): 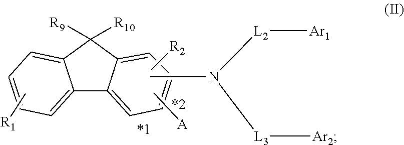
where R₁-R₂ are each independently selected from hydrogen or deuterium;
R₉ and R₁₀ are independently selected from substituted or unsubstituted straight or branched C1-C30 alkyl groups, substituted or unsubstituted C6-C30 aryl groups; or, R₉ and R₁₀ are bonded to adjacent groups to form a ring;
group A is selected from 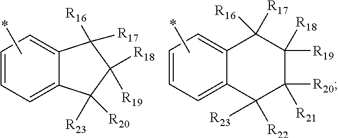
R₁₆-R₂₃ are each independently selected one or more from hydrogen, deuterium, substituted or unsubstituted C1-C60 alkyl groups, substituted or unsubstituted C1-C60 cycloalkyl groups, substituted or unsubstituted C1-C60 heteroalkyl groups, substituted or unsubstituted C1-C60 heterocycloalkyl groups, substituted or unsubstituted C1-C60 aryl groups, or substituted or unsubstituted C1-C60 heteroaryl groups; [symbol]
is a connection site of an atom, a single link or multiple links are included at the connection site; the connection site is in a ring of group A, or represents any position of the shown groups, or represents bonding with adjacent atoms to form a ring;
L₂-L₃ are each independently selected from a single bond, a substituted or unsubstituted phenylene group, a substituted or unsubstituted naphthylene group, a substituted or unsubstituted biphenylene group, a substituted or unsubstituted terphenylene group, a substituted or unsubstituted anthrylene group; and
Ar₁ and Ar₂ are the same or different, and are each independently selected from substituted or unsubstituted C6-C30 aryl groups, or substituted or unsubstituted C6-C30 heteroaryl groups.

2. The compound of claim 1, wherein it is selected from following chemical structural formulas indicated by formula (III) to (VI): 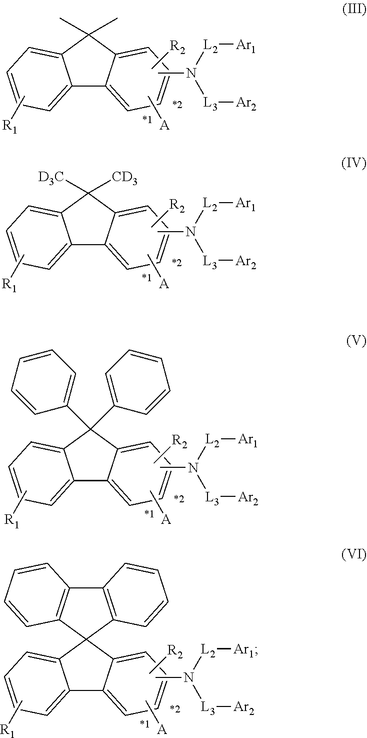
where R₁, R₂, A, L₂, L₃, Ar₁, and Ar₂ are as defined as in claim 1.

3. The compound of claim 2, wherein ring A is selected from following structures:

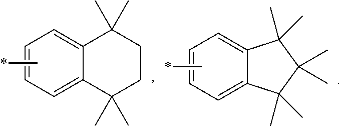

4. The compound of claim 1, wherein the chemical structure of the compound is as shown in formula (VII): 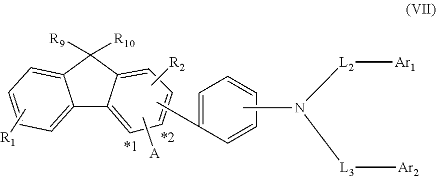
where R₁ and R₂ are each independently selected from hydrogen or deuterium;
R₉, R₁₀, A, L₂, L₃, Ar₁, and Ar₂ are as defined in claim 1.

5. The compound of claim 4, wherein the chemical structure of the compound is as shown in formulas (VIII) to (XI): 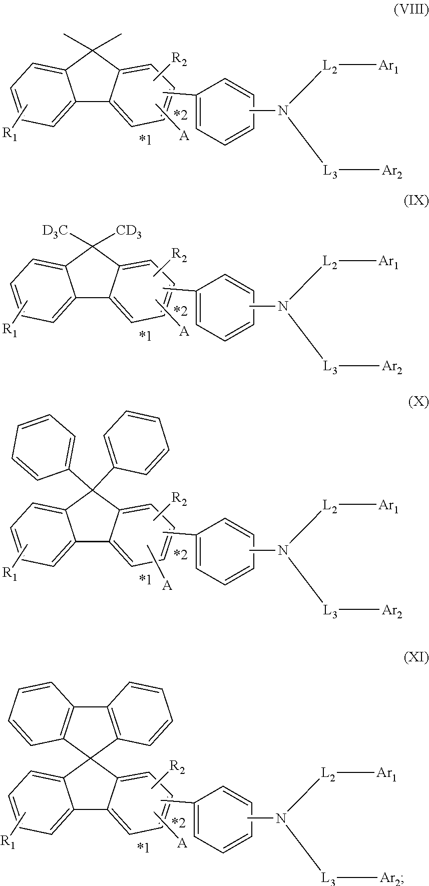
where R₁, R₂, A, L₂, L₃, Ar₁, and Ar₂ are as defined in claim 4.

6. The compound of claim 1, wherein Ar₁ and Ar₂ are each independently selected from a substituted or unsubstituted phenyl group, a substituted or unsubstituted naphthyl group, a substituted or unsubstituted biphenyl group, a substituted or unsubstituted terphenyl group,

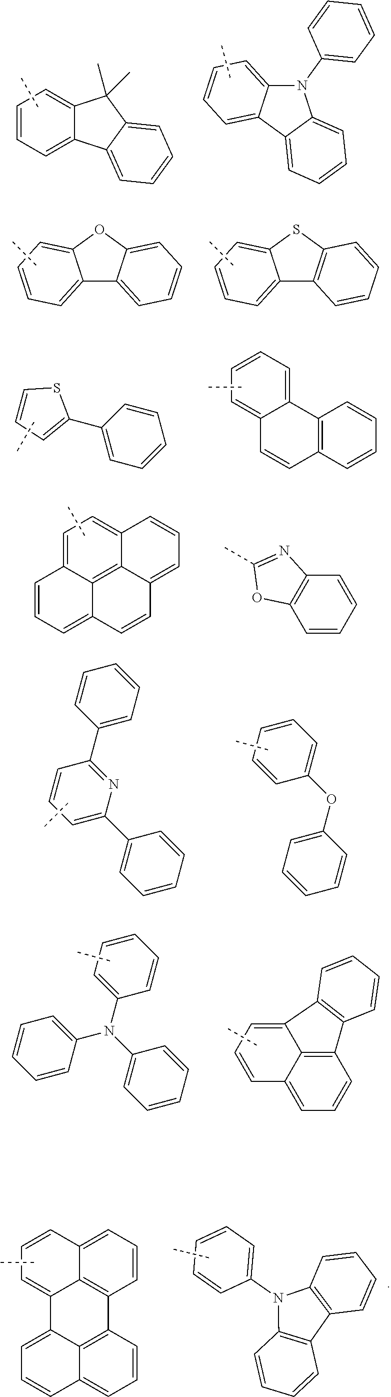

7. The compound of claim 1, wherein the compound is any one selected from following chemical structures:

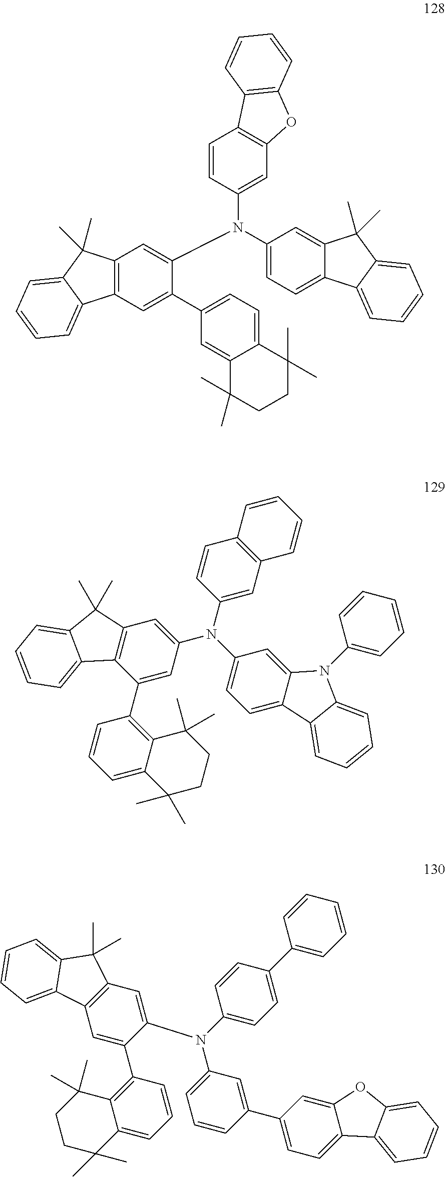 [SMILES: CC1(C)CCC(C)(C)c2cc(-c3cc4c(cc3N(c3ccc5c(c3)C(C)(C)c3ccccc3-5)c3ccc5c(c3)oc3ccccc35)C(C)(C)c3ccccc3-4)ccc21 ; CC1(C)CCC(C)(C)c2c(-c3cc(N(c4ccc5ccccc5c4)c4ccc5c6ccccc6n(-c6ccccc6)c5c4)cc4c3-c3ccccc3C4(C)C)cccc21 ; CC1(C)CCC(C)(C)c2c(-c3cc4c(cc3N(c3ccc(-c5ccccc5)cc3)c3cccc(-c5ccc6c(c5)oc5ccccc56)c3)C(C)(C)c3ccccc3-4)cccc21]

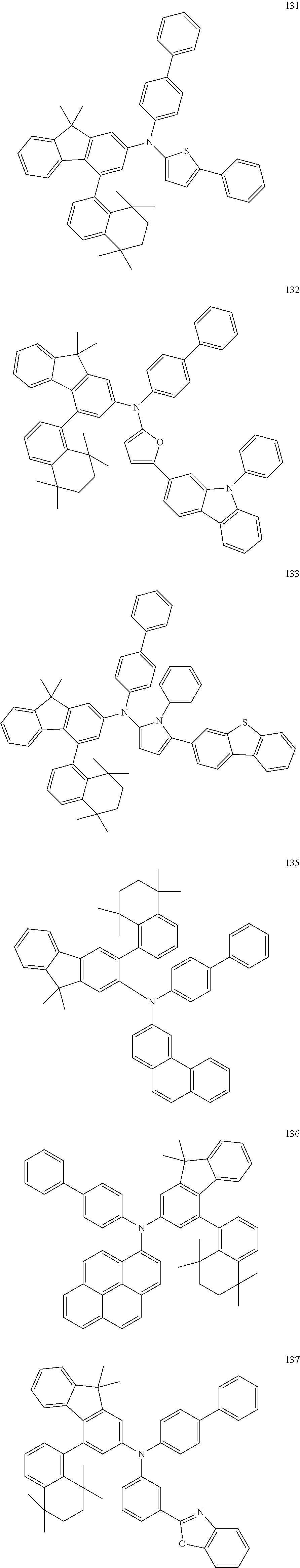

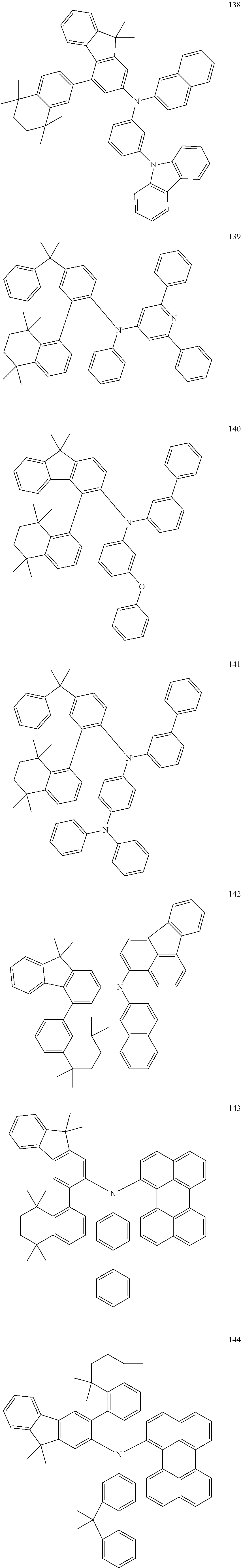

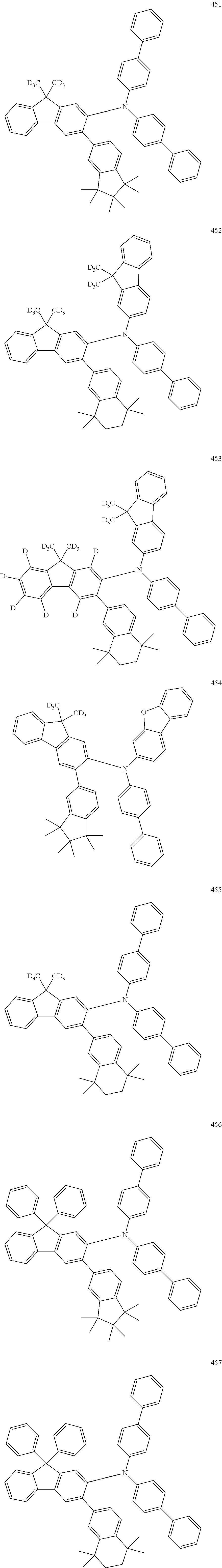

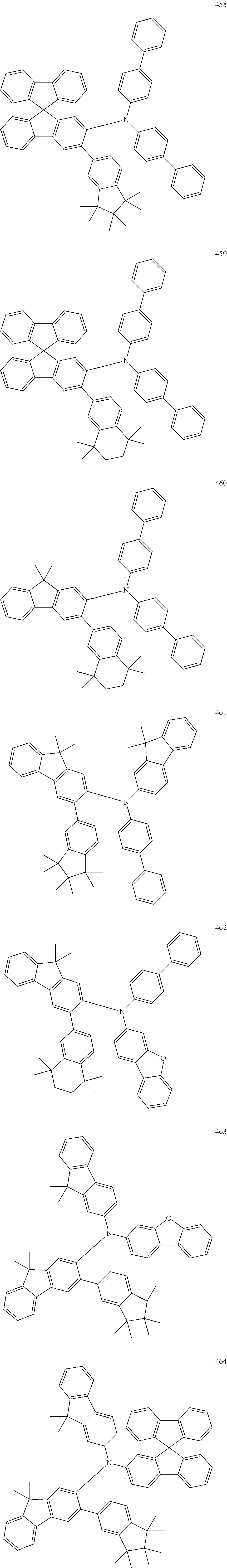

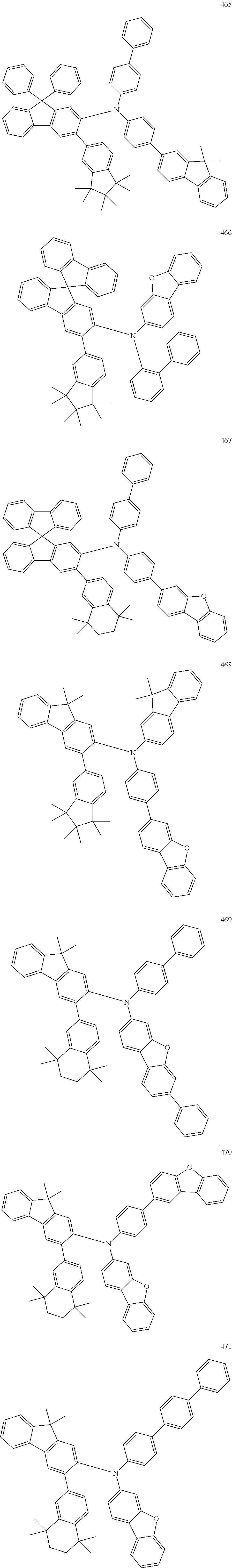

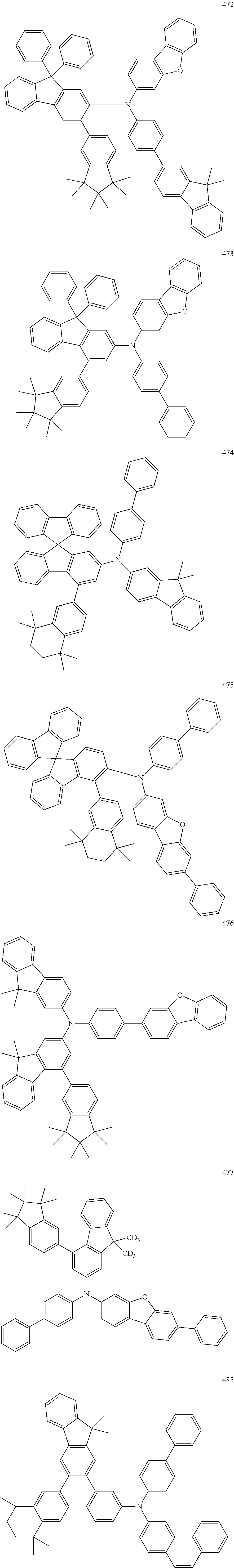

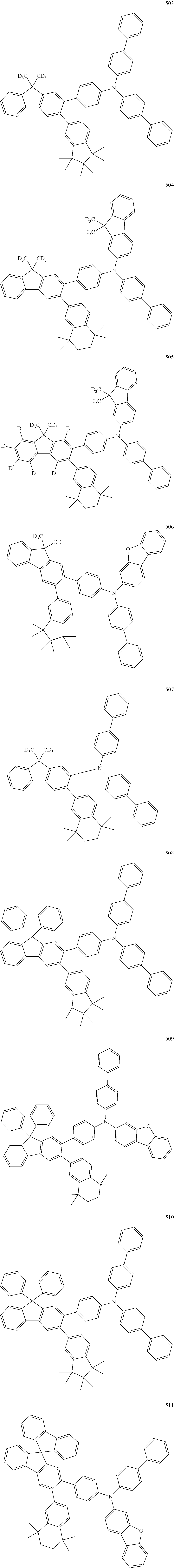

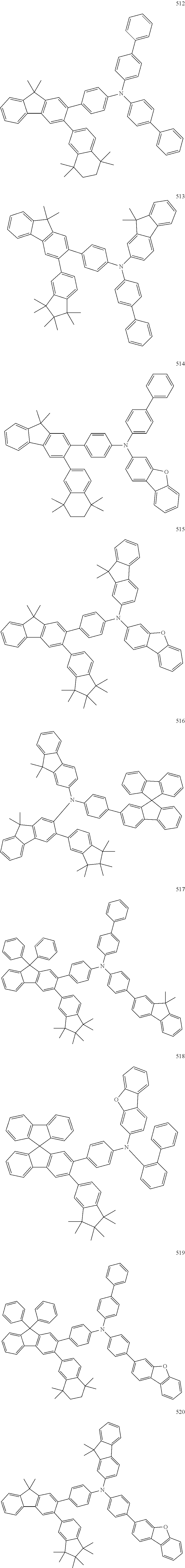

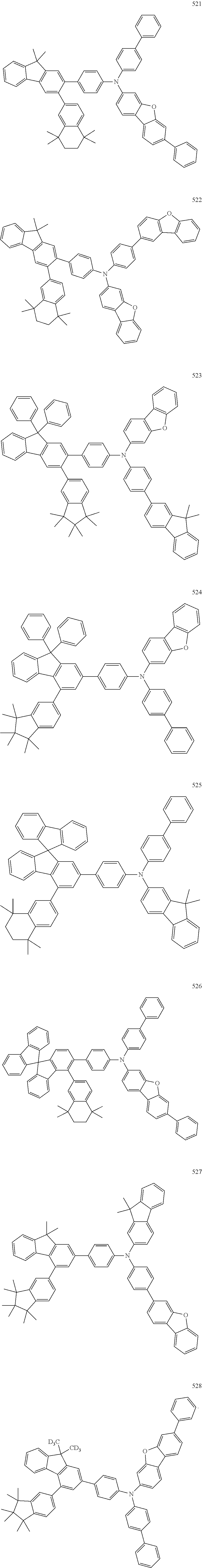

8. The compound of claim 3, wherein the chemical structure of the compound is selected from following structures: 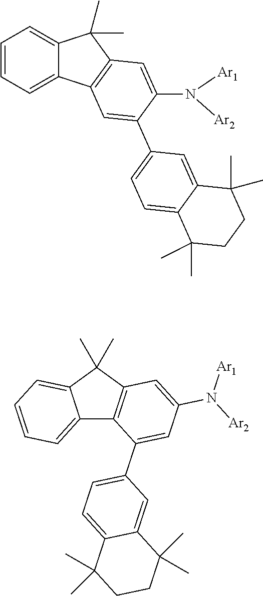
where Ar₁ and Ar₂ are each independently selected from phenyl, diphenyl, terphenyl, naphthyl, phenanthryl, dimethylfluorene, diphenylfluorene, spirofluorene, dibenzothienyl, dibenzofuranyl, carbazolyl, or a combination of any two of these groups;
when Ar₁ and Ar₂ are selected from dimethylfluorene, diphenylfluorene, and spirofluorene, their adjacent N position is not connected with other groups;
Ar₁ and Ar₂ are not selected as a combination of diphenyl and carbazolyl.

9. A compound, wherein a chemical structure of the compound is shown in formula (I): 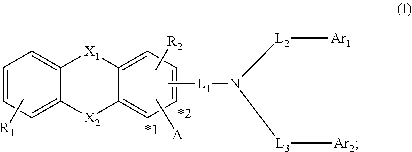
where group A is one or more selected from of the following groups: 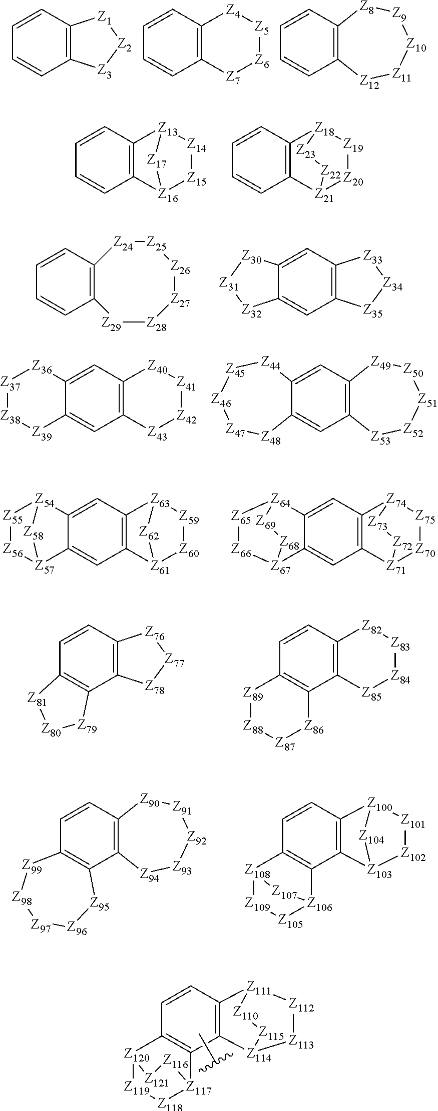
Z₁-Z₇₅, Z₇₆—Z₁₂₁ are each independently selected from CR₃R₄, NR₅, SiR₆R₇, BRs, O or S; *1, *2 are connection sites of group A; *1 or *2 is capable of being connected to any position on group A;
X₁, X₂ are each independently selected from a single bond, CR₉R₁₀, NR₁₁, SiR₁₂R₁₃, O or S; or R₉, R₁₀ are bonded to form a ring;
R₁-R₃ are the same or different, and are each independently selected from hydrogen, deuterium, substituted or unsubstituted straight or branched C1-C30 alkyl groups, substituted or unsubstituted C1-C30 heteroalkyl groups, substituted or unsubstituted C3-C30 cycloalkyl groups, substituted or unsubstituted C3-C30 heterocycloalkyl groups, substituted or unsubstituted C6-C30 aryl groups, or substituted or unsubstituted C6-C30 heteroaryl groups;
L₁-L₃ are the same or different, and are each independently selected from a single bond, substituted or unsubstituted C6-C30 arylene groups, or substituted or unsubstituted C3-C30 heteroarylene groups;
Ar₁ and Ar₂ are the same or different, and are each independently selected from substituted or unsubstituted C6-C30 aryl groups, or substituted or unsubstituted C6-C30 heteroaryl groups.

10. The compound of claim 9, wherein group A is any one selected from following groups: 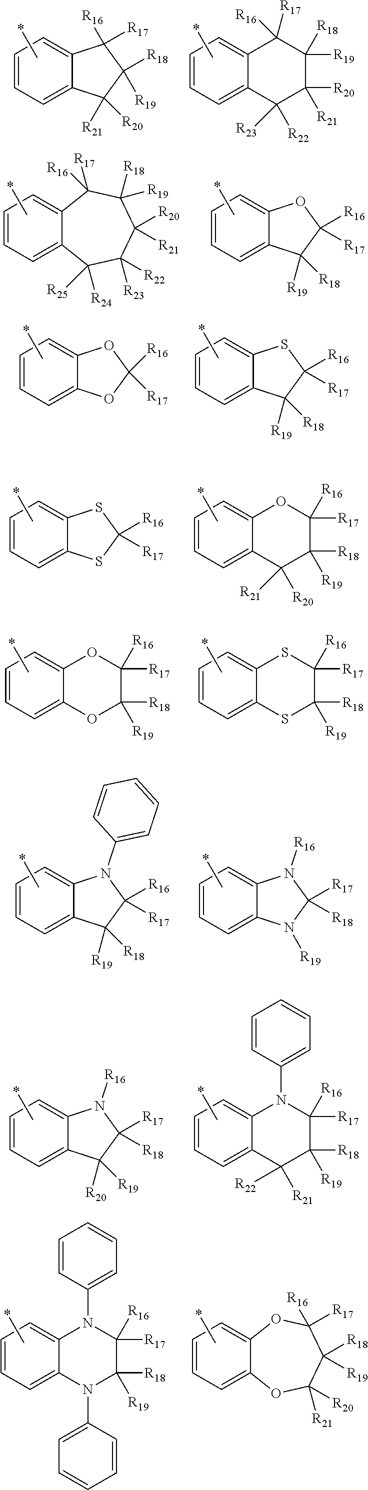 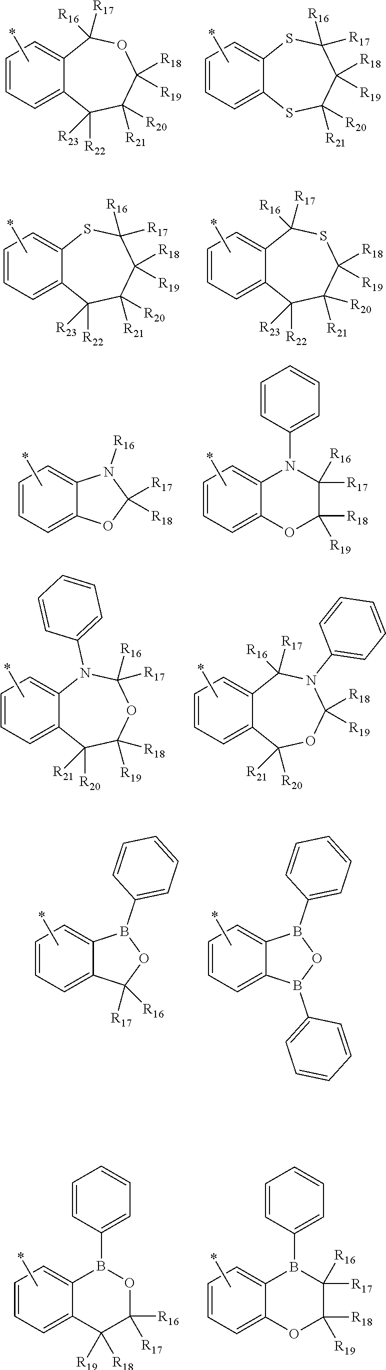 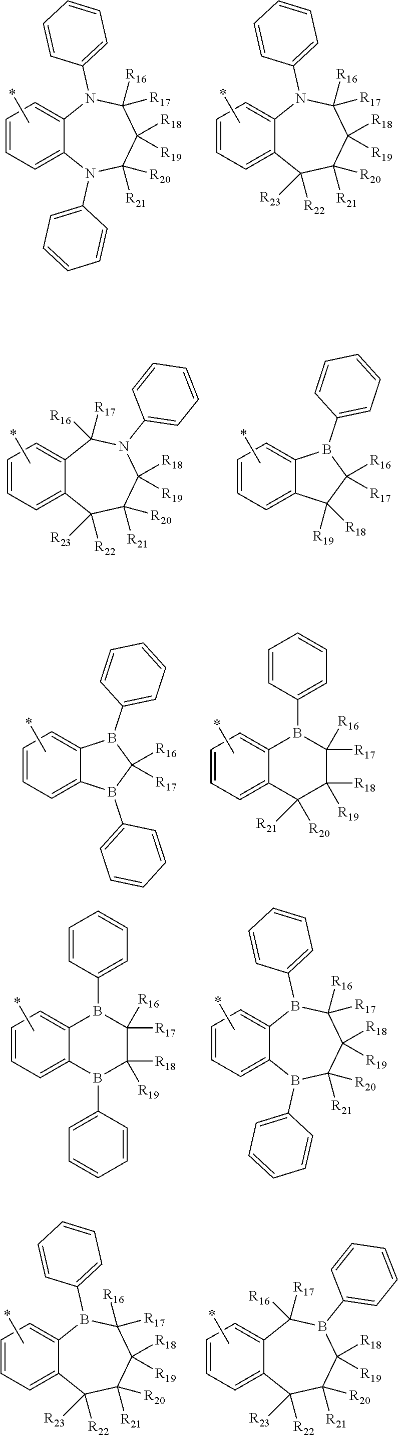
where R₁₆-R₂₃ are each independently selected one or more from hydrogen, deuterium, substituted or unsubstituted C1-C60 alkyl groups, substituted or unsubstituted C1-C60 cycloalkyl groups, substituted or unsubstituted C1-C60 heteroalkyl groups, substituted or unsubstituted C1-C60 heterocycloalkyl groups, substituted or unsubstituted C1-C60 aryl groups, or substituted or unsubstituted C1-C60 heteroaryl groups; [symbol]
is a connection site of an atom.

11. The compound of claim 9, wherein group A is any one selected from following groups:

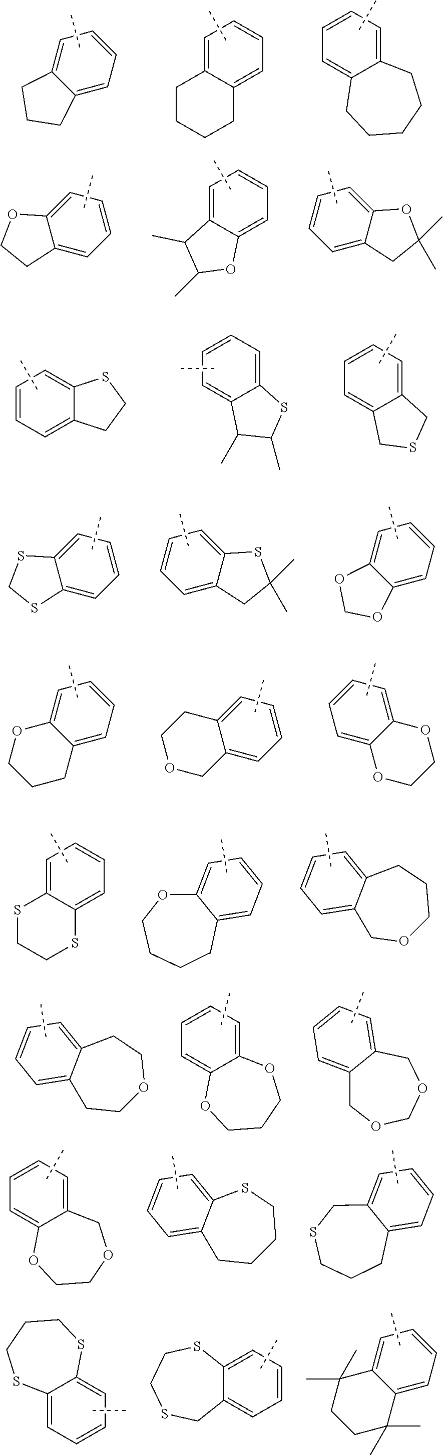

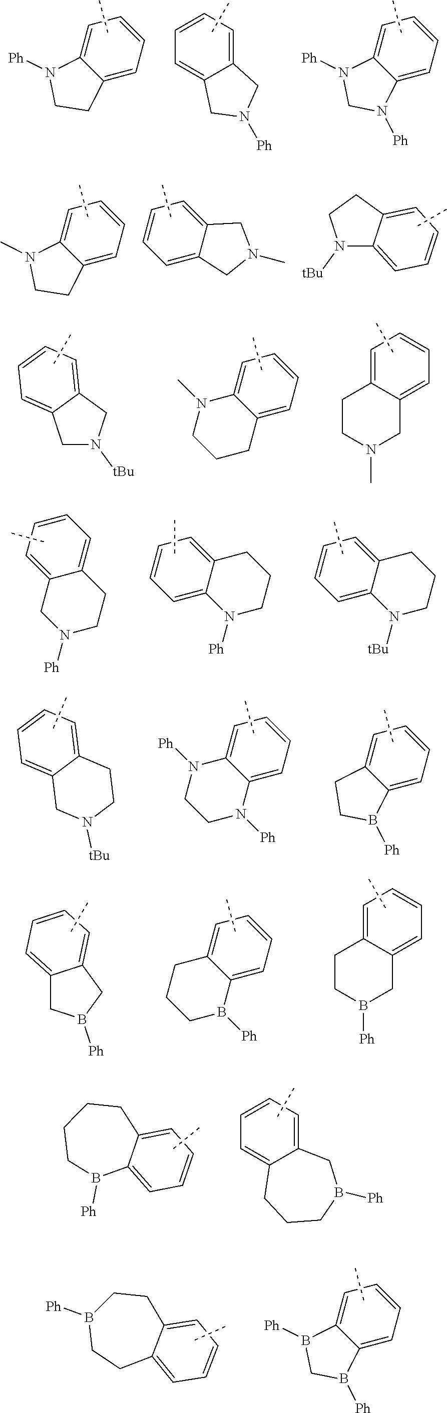

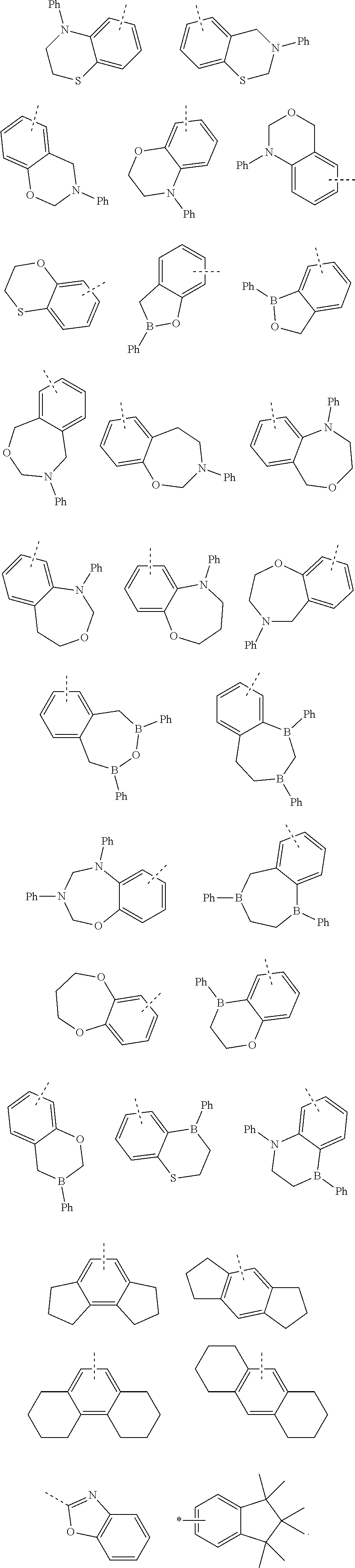

12. The compound of claim 9, wherein X₁ and X₂ are each independently selected from a single bond, O, S,

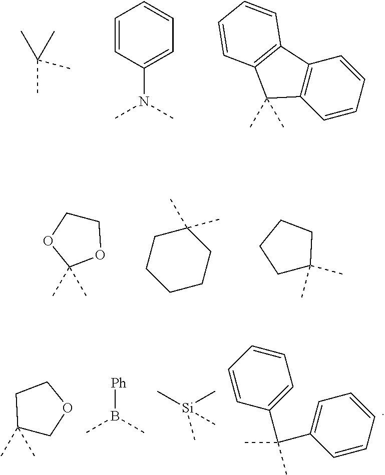

13. The compound of claim 9, wherein R₁ and R₂ are each independently selected from hydrogen or deuterium; and/or, R₃-R₁₃ are the same or different, and are each independently selected from hydrogen, deuterium, substituted or unsubstituted straight or branched C1-C30 alkyl groups, substituted or unsubstituted C1-C12 alkoxy groups, substituted or unsubstituted C1-C12 alkylthio groups, substituted or unsubstituted C3-C30 cycloalkyl groups, substituted or unsubstituted C3-C30 heterocycloalkyl groups, substituted or unsubstituted C6-C30 aryl groups, or substituted or unsubstituted C6-C30 heteroaryl groups;
wherein L₁-L₃ are each independently selected from a single bond, a substituted or unsubstituted phenylene group, a substituted or unsubstituted phenylene group, a substituted or unsubstituted naphthylene group, a substituted or unsubstituted biphenylene group, a substituted or unsubstituted terphenylene group, a substituted or unsubstituted anthrylene group, a substituted or unsubstituted phenanthrenyl group, 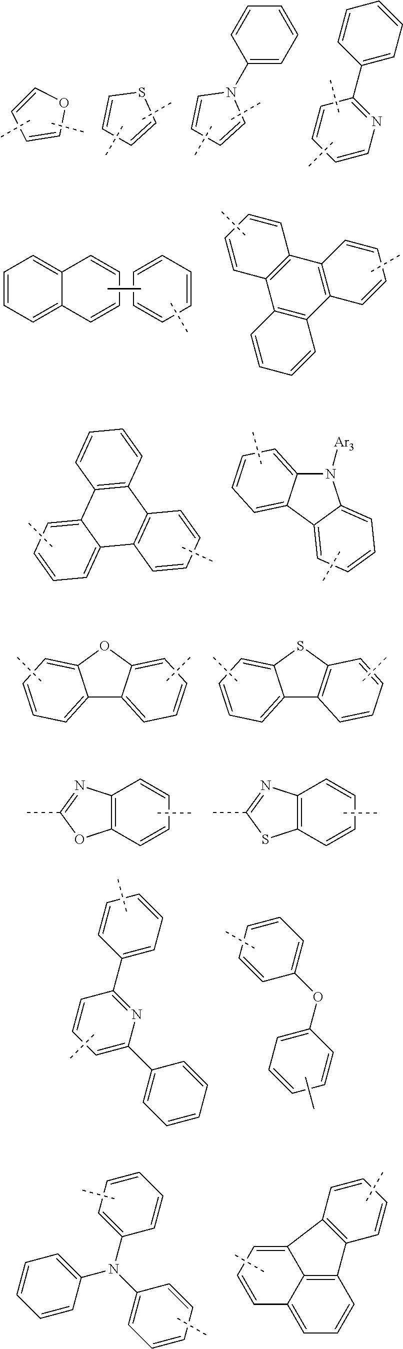 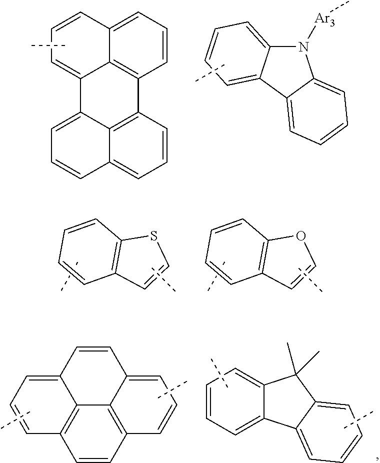
and Ar₃ is selected from C6-C20 aryl groups or C2-C15 heteroaryl groups;
wherein Ar₁ and Ar₂ are each independently selected from a substituted or unsubstituted phenyl group, a substituted or unsubstituted naphthyl group, a substituted or unsubstituted biphenyl group, a substituted or unsubstituted terphenyl group,

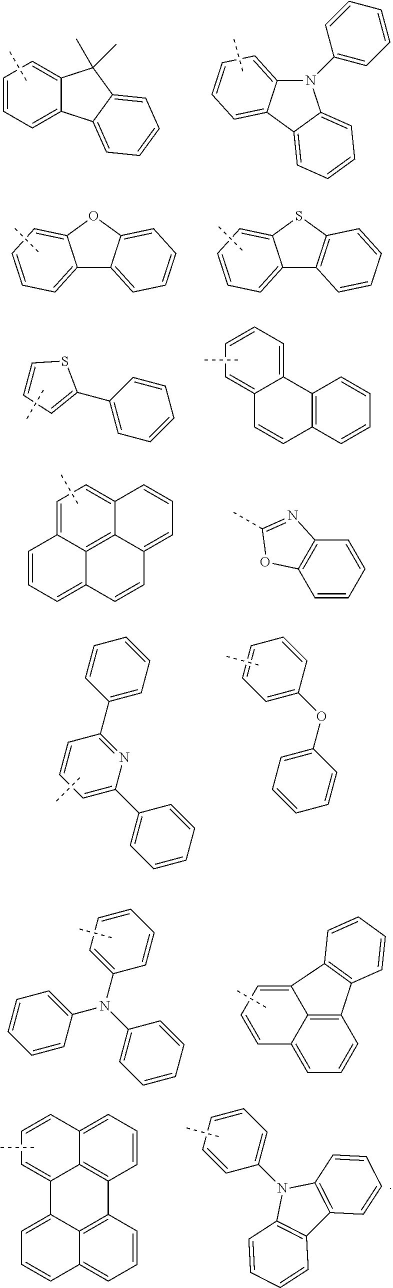

14. The compound of claim 9, wherein the chemical structure of the compound is as shown in formula (II): 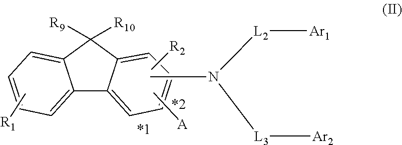
where R₁ and R₂ are each independently selected from hydrogen or deuterium;
R₉ and R₁₀ are independently selected from substituted or unsubstituted straight or branched C1-C30 alkyl groups, substituted or unsubstituted C1-C12 alkoxy groups, substituted or unsubstituted C1-C12 alkylthio groups, substituted or unsubstituted C3-C30 cycloalkyl groups, substituted or unsubstituted C3-C30 heterocycloalkyl groups, substituted or unsubstituted C6-C30 aryl groups, or substituted or unsubstituted C6-C30 heteroaryl groups; or, R₉ and R₁₀ are bonded to adjacent groups to form a ring; and
A, L₂, L₃, Ar₁, and Ar₂ are as defined in claim 9.

15. The compound of claim 14, wherein it is selected from following chemical structural formulas indicated by formula (III) to (VI): 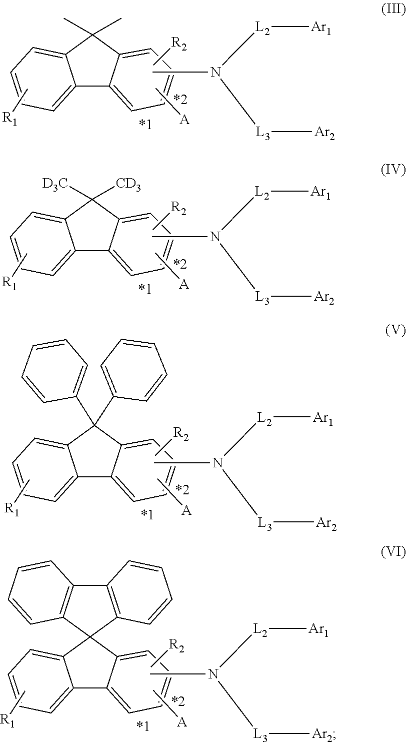
where R₁, R₂, A, L₂, L₃, Ar₁, and Ar₂ are as defined as in claim 14.

16. The compound of claim 15, wherein ring A is selected from following structures:

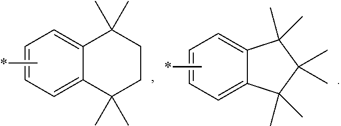

17. The compound of claim 9, wherein the chemical structure of the compound is as shown in formula (VII): 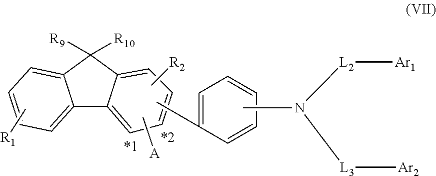
where R₁ and R₂ are each independently selected from hydrogen or deuterium;
R₉ and R₁₀ are independently selected from substituted or unsubstituted straight or branched C1-C30 alkyl groups, substituted or unsubstituted C1-C12 alkoxy groups, substituted or unsubstituted C1-C12 alkylthio groups, substituted or unsubstituted C3-C30 cycloalkyl groups, substituted or unsubstituted C3-C30 heterocycloalkyl groups, substituted or unsubstituted C6-C30 aryl groups, or substituted or unsubstituted C6-C30 heteroaryl groups; or, R₉ and R₁₀ are bonded to adjacent groups to form a ring; and
A, L₂, L₃, Ar₁, and Ar₂ are as defined in claim 9.

18. The compound of claim 17, wherein the chemical structure of the compound is as shown in formula (VIII) to (XI): 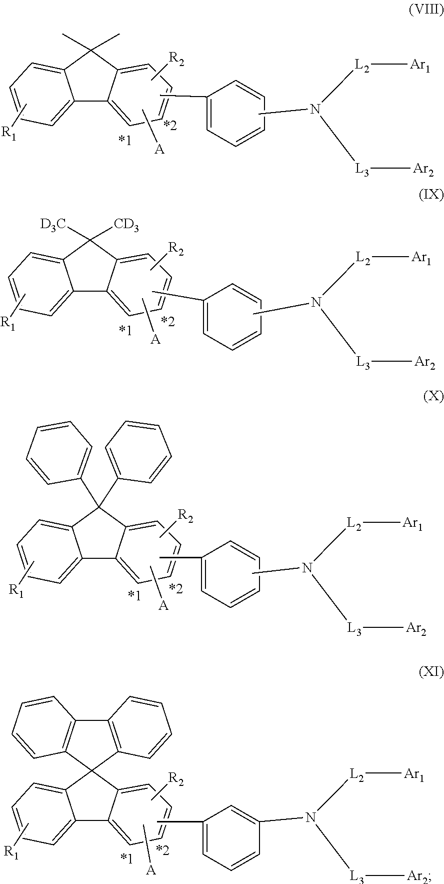
R₁, R₂, A, L₂, L₃, Ar₁, and Ar₂ are as defined in claim 17.

19. The compound of claim 9, wherein the compound is any one selected from following chemical structures:

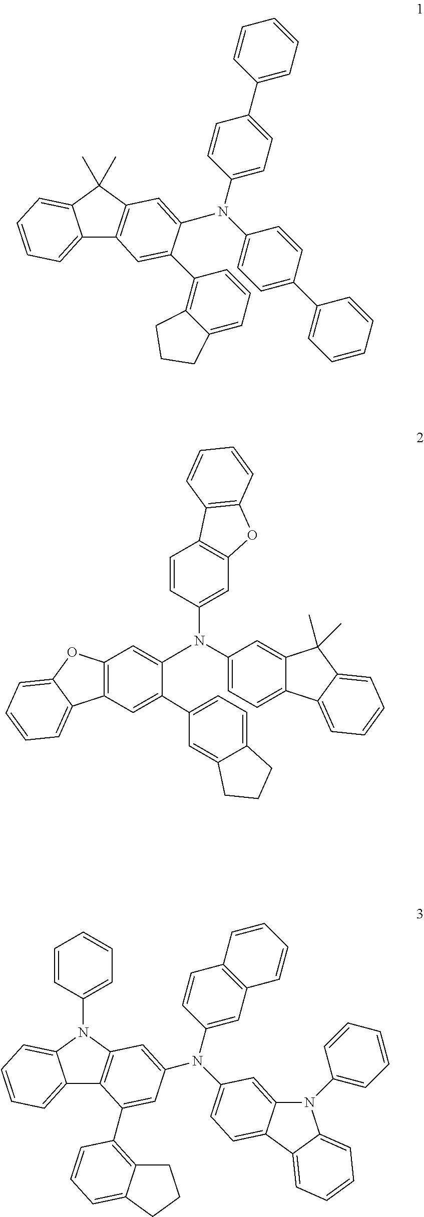 [SMILES: CC1(C)c2ccccc2-c2cc(-c3cccc4c3CCC4)c(N(c3ccc(-c4ccccc4)cc3)c3ccc(-c4ccccc4)cc3)cc21 ; CC1(C)c2ccccc2-c2ccc(N(c3ccc4c(c3)oc3ccccc34)c3cc4oc5ccccc5c4cc3-c3ccc4c(c3)CCC4)cc21 ; c1ccc(-n2c3ccccc3c3ccc(N(c4ccc5ccccc5c4)c4cc(-c5cccc6c5CCC6)c5c6ccccc6n(-c6ccccc6)c5c4)cc32)cc1]

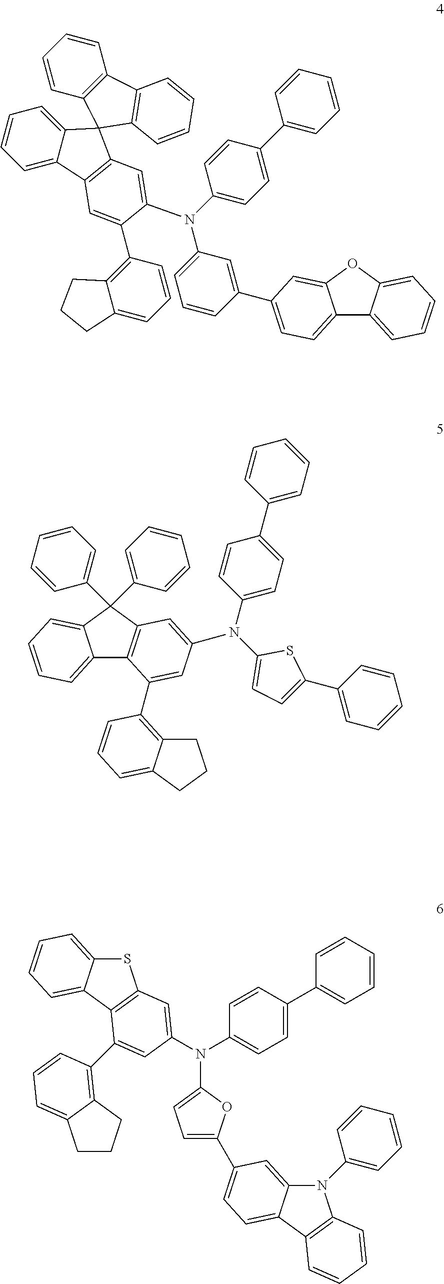 [SMILES: c1ccc(-c2ccc(N(c3cccc(-c4ccc5c(c4)oc4ccccc45)c3)c3cc4c(cc3-c3cccc5c3CCC5)-c3ccccc3C43c4ccccc4-c4ccccc43)cc2)cc1 ; c1ccc(-c2ccc(N(c3cc(-c4cccc5c4CCC5)c4c(c3)C(c3ccccc3)(c3ccccc3)c3ccccc3-4)c3ccc(-c4ccccc4)s3)cc2)cc1 ; c1ccc(-c2ccc(N(c3cc(-c4cccc5c4CCC5)c4c(c3)sc3ccccc34)c3ccc(-c4ccc5c6ccccc6n(-c6ccccc6)c5c4)o3)cc2)cc1]

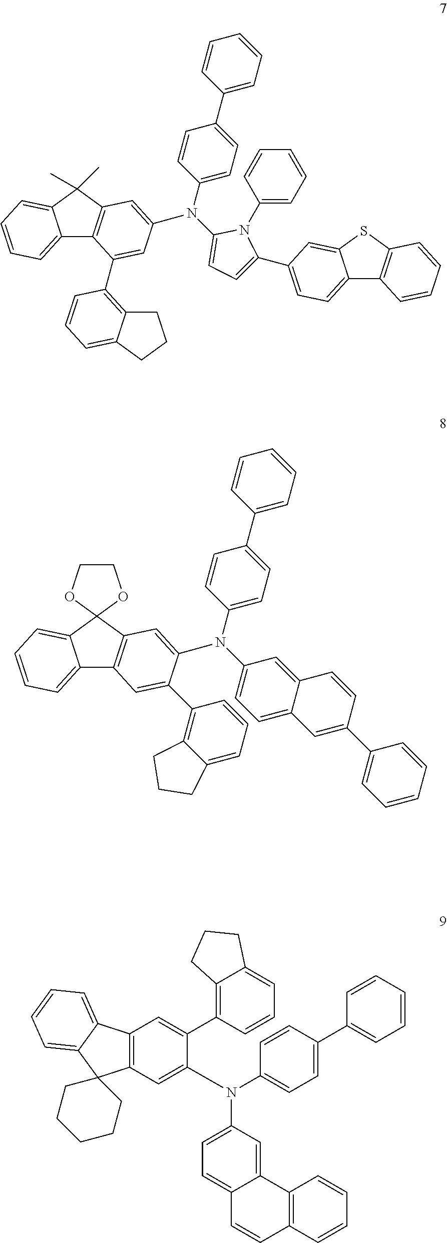 [SMILES: CC1(C)c2ccccc2-c2c(-c3cccc4c3CCC4)cc(N(c3ccc(-c4ccccc4)cc3)c3ccc(-c4ccc5c(c4)sc4ccccc45)n3-c3ccccc3)cc21 ; c1ccc(-c2ccc(N(c3ccc4cc(-c5ccccc5)ccc4c3)c3cc4c(cc3-c3cccc5c3CCC5)-c3ccccc3C43OCCO3)cc2)cc1 ; c1ccc(-c2ccc(N(c3ccc4ccc5ccccc5c4c3)c3cc4c(cc3-c3cccc5c3CCC5)-c3ccccc3C43CCCCC3)cc2)cc1]

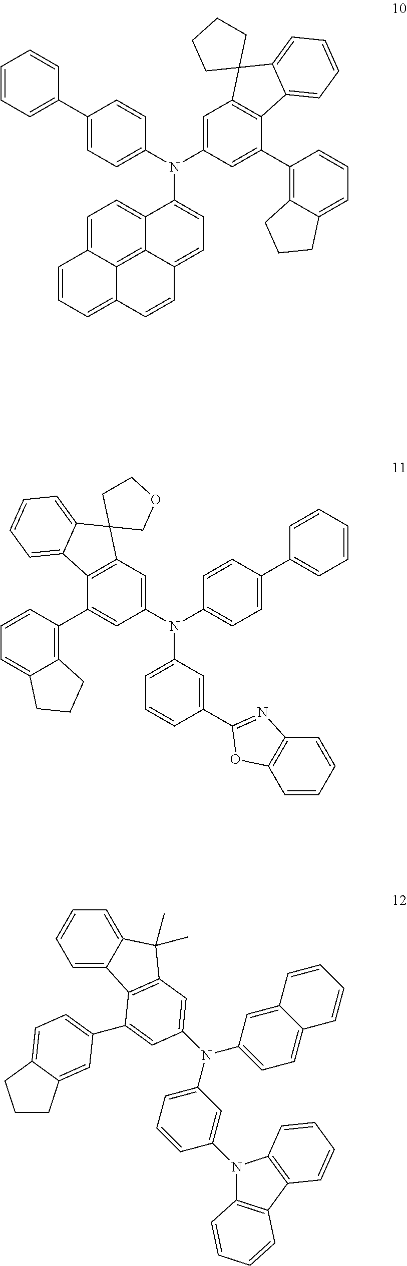 [SMILES: c1ccc(-c2ccc(N(c3cc(-c4cccc5c4CCC5)c4c(c3)C3(CCCC3)c3ccccc3-4)c3ccc4ccc5cccc6ccc3c4c56)cc2)cc1 ; c1ccc(-c2ccc(N(c3cccc(-c4nc5ccccc5o4)c3)c3cc(-c4cccc5c4CCC5)c4c(c3)C3(CCOC3)c3ccccc3-4)cc2)cc1 ; CC1(C)c2ccccc2-c2c(-c3ccc4c(c3)CCC4)cc(N(c3cccc(-n4c5ccccc5c5ccccc54)c3)c3ccc4ccccc4c3)cc21]

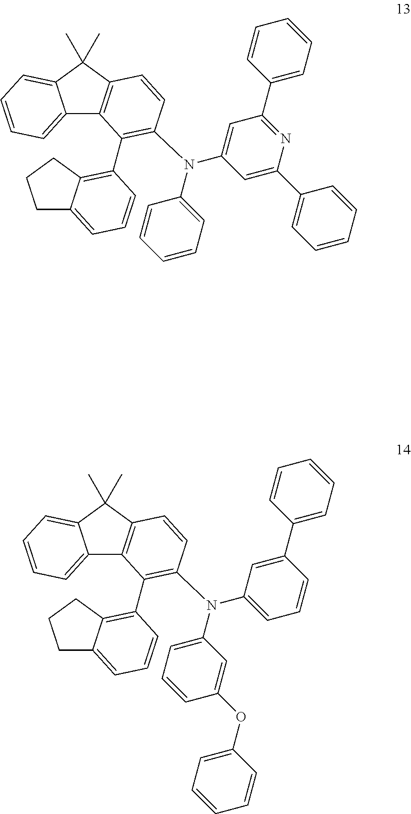 [SMILES: CC1(C)c2ccccc2-c2c1ccc(N(c1ccccc1)c1cc(-c3ccccc3)nc(-c3ccccc3)c1)c2-c1cccc2c1CCC2 ; CC1(C)c2ccccc2-c2c1ccc(N(c1cccc(Oc3ccccc3)c1)c1cccc(-c3ccccc3)c1)c2-c1cccc2c1CCC2]

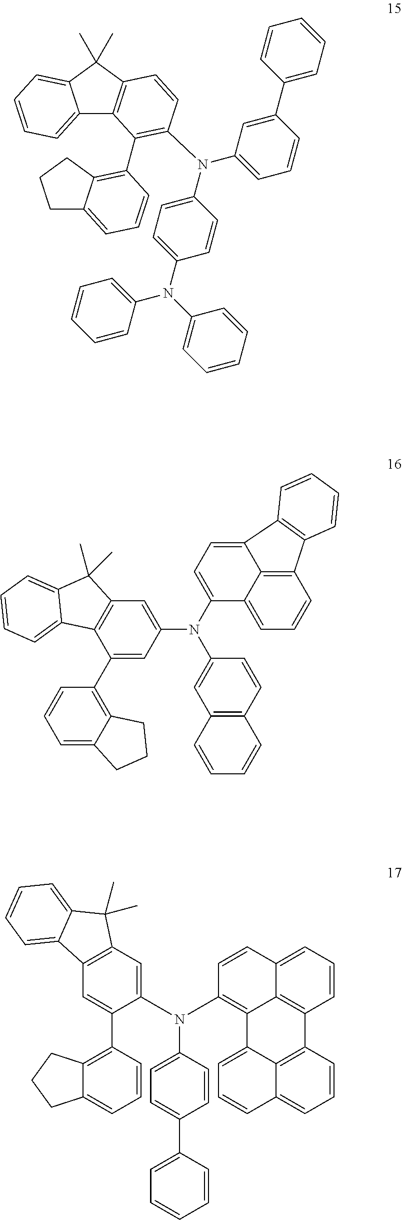 [SMILES: CC1(C)c2ccccc2-c2c1ccc(N(c1ccc(N(c3ccccc3)c3ccccc3)cc1)c1cccc(-c3ccccc3)c1)c2-c1cccc2c1CCC2 ; CC1(C)c2ccccc2-c2c(-c3cccc4c3CCC4)cc(N(c3ccc4ccccc4c3)c3ccc4c5c(cccc35)-c3ccccc3-4)cc21 ; CC1(C)c2ccccc2-c2cc(-c3cccc4c3CCC4)c(N(c3ccc(-c4ccccc4)cc3)c3ccc4cccc5c6cccc7cccc(c3c45)c76)cc21]

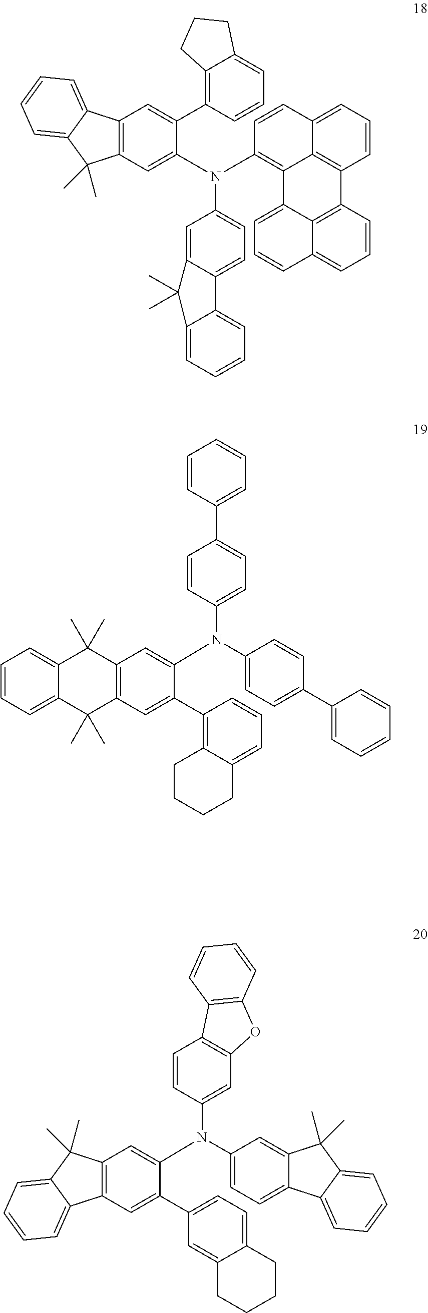 [SMILES: CC1(C)c2ccccc2-c2ccc(N(c3cc4c(cc3-c3cccc5c3CCC5)-c3ccccc3C4(C)C)c3ccc4cccc5c6cccc7cccc(c3c45)c76)cc21 ; CC1(C)c2ccccc2C(C)(C)c2cc(N(c3ccc(-c4ccccc4)cc3)c3ccc(-c4ccccc4)cc3)c(-c3cccc4c3CCCC4)cc21 ; CC1(C)c2ccccc2-c2ccc(N(c3ccc4c(c3)oc3ccccc34)c3cc4c(cc3-c3ccc5c(c3)CCCC5)-c3ccccc3C4(C)C)cc21]

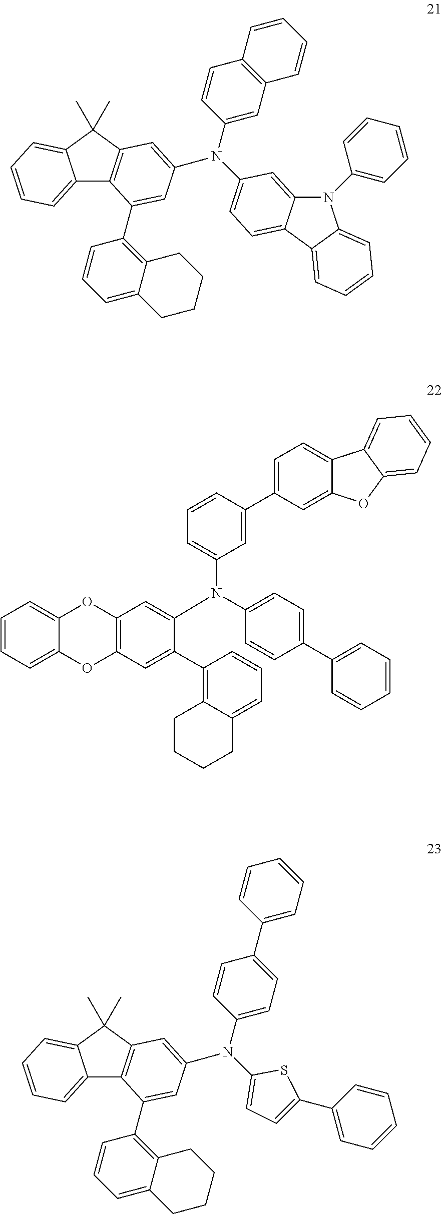 [SMILES: CC1(C)c2ccccc2-c2c(-c3cccc4c3CCCC4)cc(N(c3ccc4ccccc4c3)c3ccc4c5ccccc5n(-c5ccccc5)c4c3)cc21 ; c1ccc(-c2ccc(N(c3cccc(-c4ccc5c(c4)oc4ccccc45)c3)c3cc4c(cc3-c3cccc5c3CCCC5)Oc3ccccc3O4)cc2)cc1 ; CC1(C)c2ccccc2-c2c(-c3cccc4c3CCCC4)cc(N(c3ccc(-c4ccccc4)cc3)c3ccc(-c4ccccc4)s3)cc21]

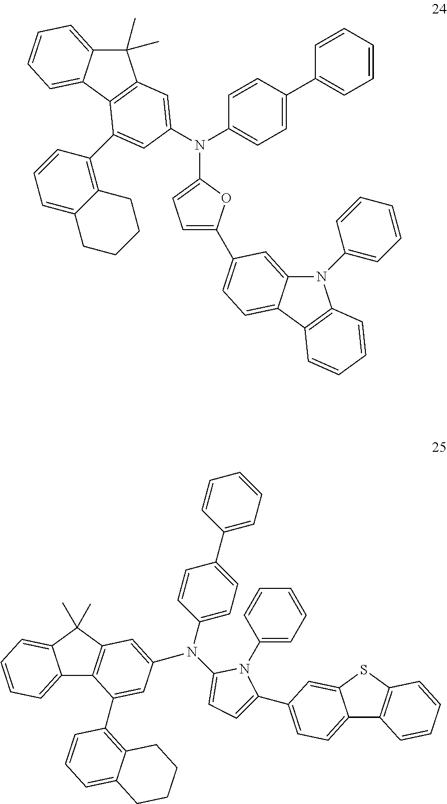 [SMILES: CC1(C)c2ccccc2-c2c(-c3cccc4c3CCCC4)cc(N(c3ccc(-c4ccccc4)cc3)c3ccc(-c4ccc5c6ccccc6n(-c6ccccc6)c5c4)o3)cc21 ; CC1(C)c2ccccc2-c2c(-c3cccc4c3CCCC4)cc(N(c3ccc(-c4ccccc4)cc3)c3ccc(-c4ccc5c(c4)sc4ccccc45)n3-c3ccccc3)cc21]

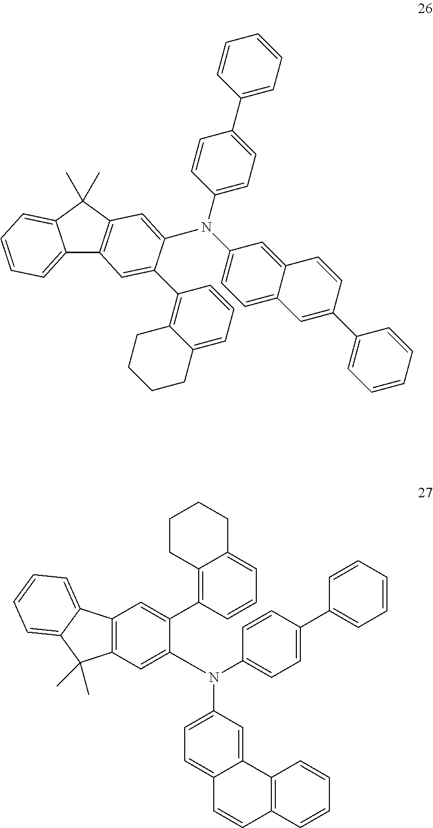 [SMILES: CC1(C)c2ccccc2-c2cc(-c3cccc4c3CCCC4)c(N(c3ccc(-c4ccccc4)cc3)c3ccc4cc(-c5ccccc5)ccc4c3)cc21 ; CC1(C)c2ccccc2-c2cc(-c3cccc4c3CCCC4)c(N(c3ccc(-c4ccccc4)cc3)c3ccc4ccc5ccccc5c4c3)cc21]

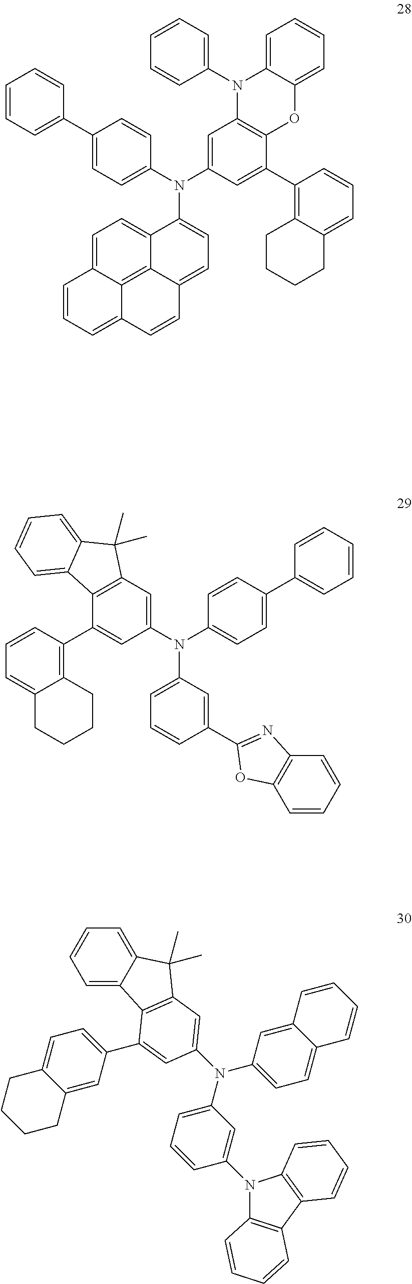 [SMILES: c1ccc(-c2ccc(N(c3cc(-c4cccc5c4CCCC5)c4c(c3)N(c3ccccc3)c3ccccc3O4)c3ccc4ccc5cccc6ccc3c4c56)cc2)cc1 ; CC1(C)c2ccccc2-c2c(-c3cccc4c3CCCC4)cc(N(c3ccc(-c4ccccc4)cc3)c3cccc(-c4nc5ccccc5o4)c3)cc21 ; CC1(C)c2ccccc2-c2c(-c3ccc4c(c3)CCCC4)cc(N(c3cccc(-n4c5ccccc5c5ccccc54)c3)c3ccc4ccccc4c3)cc21]

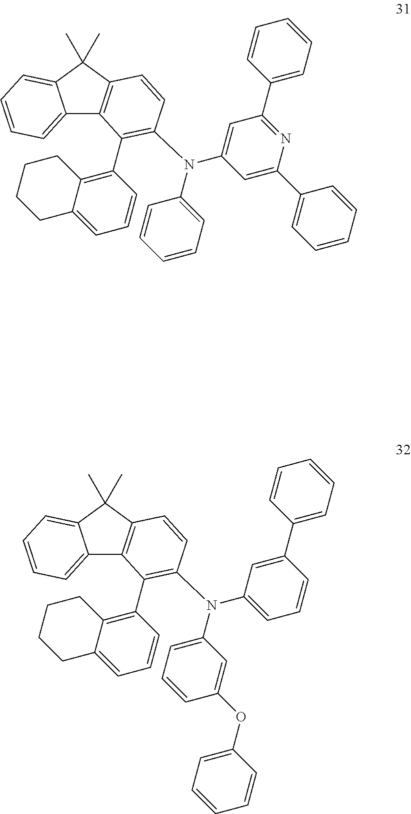 [SMILES: CC1(C)c2ccccc2-c2c1ccc(N(c1ccccc1)c1cc(-c3ccccc3)nc(-c3ccccc3)c1)c2-c1cccc2c1CCCC2 ; CC1(C)c2ccccc2-c2c1ccc(N(c1cccc(Oc3ccccc3)c1)c1cccc(-c3ccccc3)c1)c2-c1cccc2c1CCCC2]

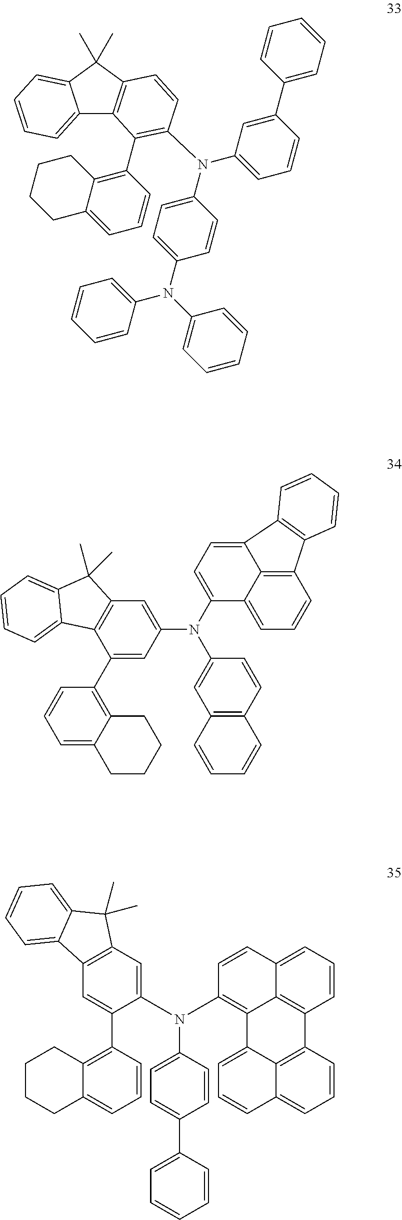 [SMILES: CC1(C)c2ccccc2-c2c1ccc(N(c1ccc(N(c3ccccc3)c3ccccc3)cc1)c1cccc(-c3ccccc3)c1)c2-c1cccc2c1CCCC2 ; CC1(C)c2ccccc2-c2c(-c3cccc4c3CCCC4)cc(N(c3ccc4ccccc4c3)c3ccc4c5c(cccc35)-c3ccccc3-4)cc21 ; CC1(C)c2ccccc2-c2cc(-c3cccc4c3CCCC4)c(N(c3ccc(-c4ccccc4)cc3)c3ccc4cccc5c6cccc7cccc(c3c45)c76)cc21]

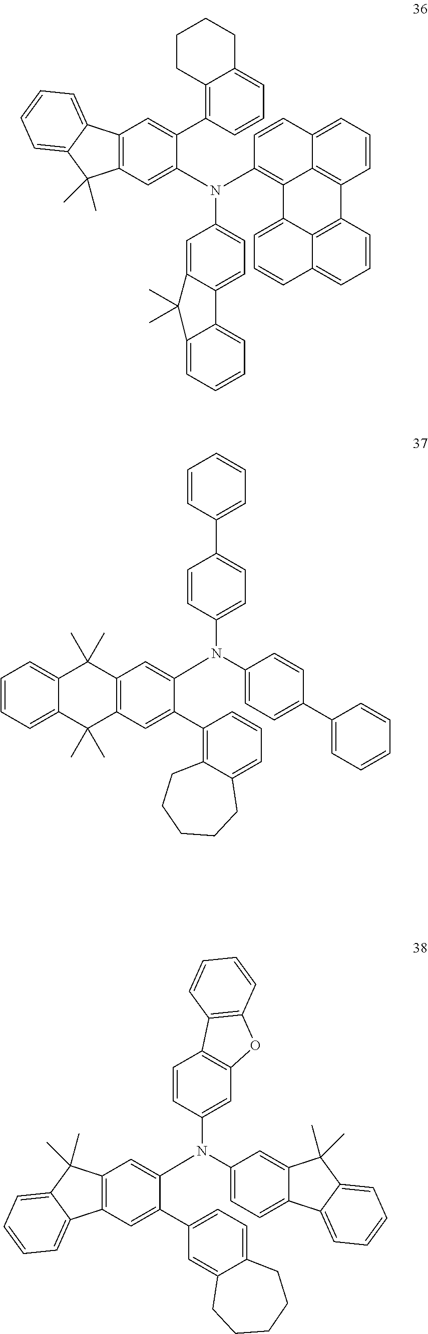 [SMILES: CC1(C)c2ccccc2-c2ccc(N(c3cc4c(cc3-c3cccc5c3CCCC5)-c3ccccc3C4(C)C)c3ccc4cccc5c6cccc7cccc(c3c45)c76)cc21 ; CC1(C)c2ccccc2C(C)(C)c2cc(N(c3ccc(-c4ccccc4)cc3)c3ccc(-c4ccccc4)cc3)c(-c3cccc4c3CCCCC4)cc21 ; CC1(C)c2ccccc2-c2ccc(N(c3ccc4c(c3)oc3ccccc34)c3cc4c(cc3-c3ccc5c(c3)CCCCC5)-c3ccccc3C4(C)C)cc21]

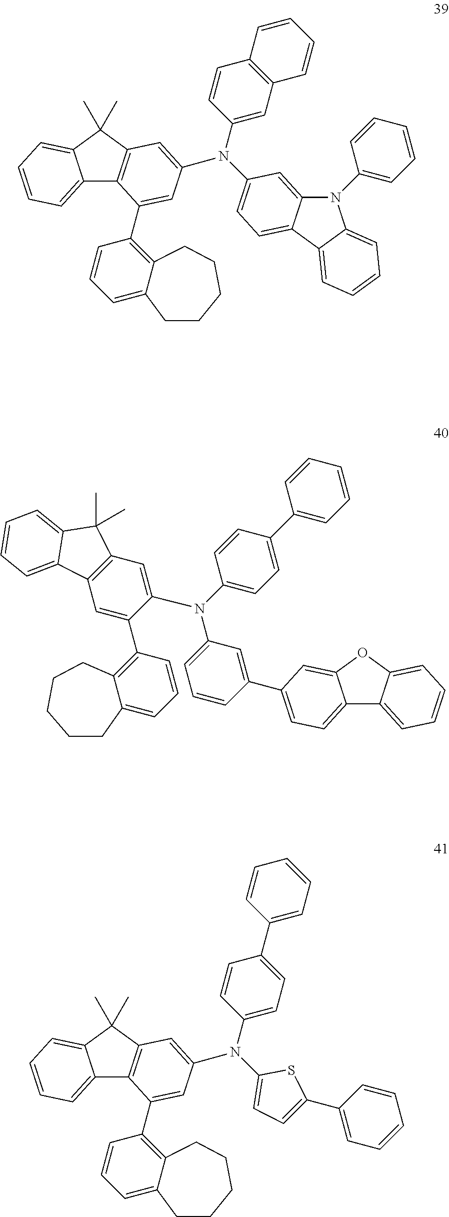 [SMILES: CC1(C)c2ccccc2-c2c(-c3cccc4c3CCCCC4)cc(N(c3ccc4ccccc4c3)c3ccc4c5ccccc5n(-c5ccccc5)c4c3)cc21 ; CC1(C)c2ccccc2-c2cc(-c3cccc4c3CCCCC4)c(N(c3ccc(-c4ccccc4)cc3)c3cccc(-c4ccc5c(c4)oc4ccccc45)c3)cc21 ; CC1(C)c2ccccc2-c2c(-c3cccc4c3CCCCC4)cc(N(c3ccc(-c4ccccc4)cc3)c3ccc(-c4ccccc4)s3)cc21]

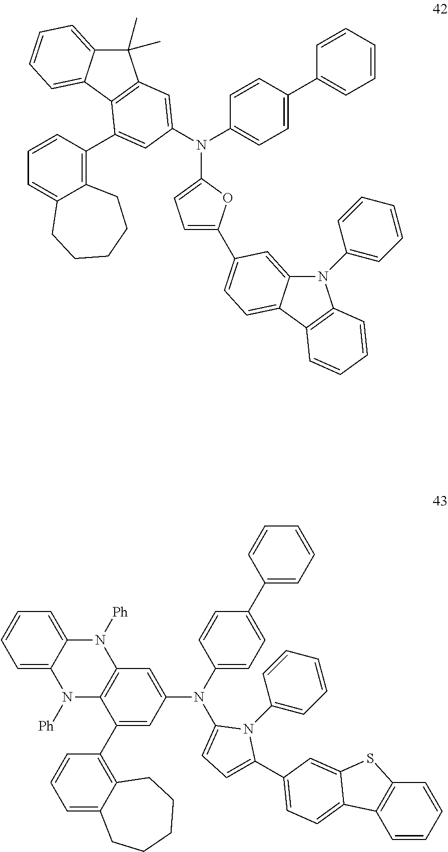 [SMILES: CC1(C)c2ccccc2-c2c(-c3cccc4c3CCCCC4)cc(N(c3ccc(-c4ccccc4)cc3)c3ccc(-c4ccc5c6ccccc6n(-c6ccccc6)c5c4)o3)cc21 ; c1ccc(-c2ccc(N(c3cc(-c4cccc5c4CCCCC5)c4c(c3)N(c3ccccc3)c3ccccc3N4c3ccccc3)c3ccc(-c4ccc5c(c4)sc4ccccc45)n3-c3ccccc3)cc2)cc1]

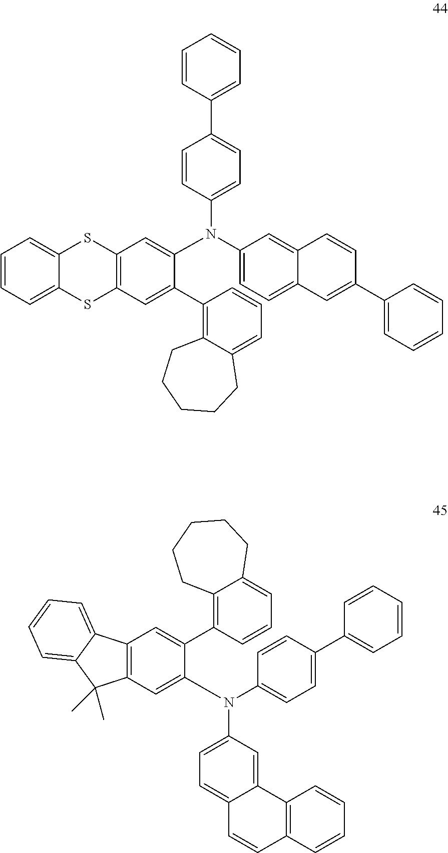 [SMILES: c1ccc(-c2ccc(N(c3ccc4cc(-c5ccccc5)ccc4c3)c3cc4c(cc3-c3cccc5c3CCCCC5)Sc3ccccc3S4)cc2)cc1 ; CC1(C)c2ccccc2-c2cc(-c3cccc4c3CCCCC4)c(N(c3ccc(-c4ccccc4)cc3)c3ccc4ccc5ccccc5c4c3)cc21]

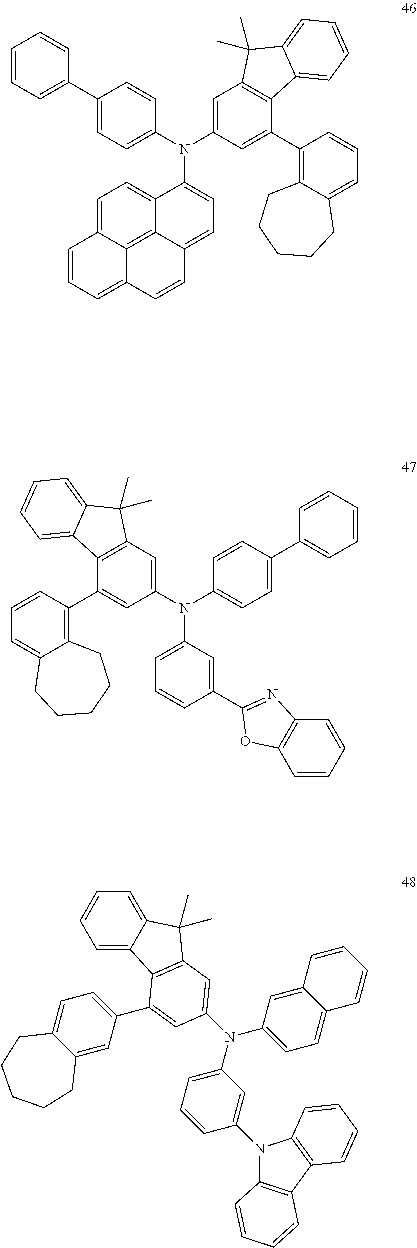 [SMILES: CC1(C)c2ccccc2-c2c(-c3cccc4c3CCCCC4)cc(N(c3ccc(-c4ccccc4)cc3)c3ccc4ccc5cccc6ccc3c4c56)cc21 ; CC1(C)c2ccccc2-c2c(-c3cccc4c3CCCCC4)cc(N(c3ccc(-c4ccccc4)cc3)c3cccc(-c4nc5ccccc5o4)c3)cc21 ; CC1(C)c2ccccc2-c2c(-c3ccc4c(c3)CCCCC4)cc(N(c3cccc(-n4c5ccccc5c5ccccc54)c3)c3ccc4ccccc4c3)cc21]

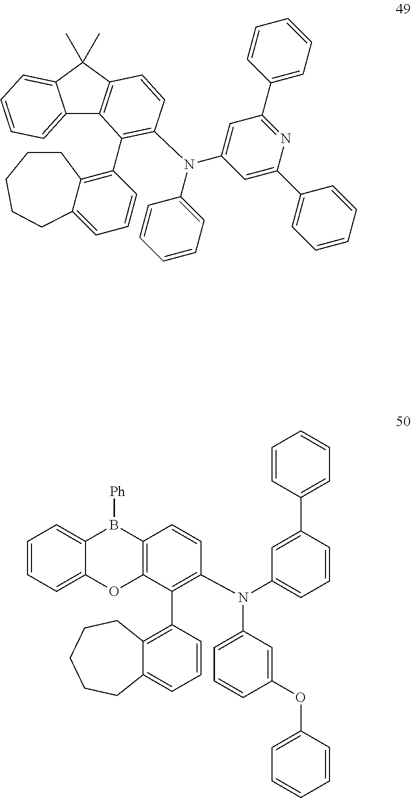 [SMILES: CC1(C)c2ccccc2-c2c1ccc(N(c1ccccc1)c1cc(-c3ccccc3)nc(-c3ccccc3)c1)c2-c1cccc2c1CCCCC2 ; c1ccc(Oc2cccc(N(c3cccc(-c4ccccc4)c3)c3ccc4c(c3-c3cccc5c3CCCCC5)Oc3ccccc3B4c3ccccc3)c2)cc1]

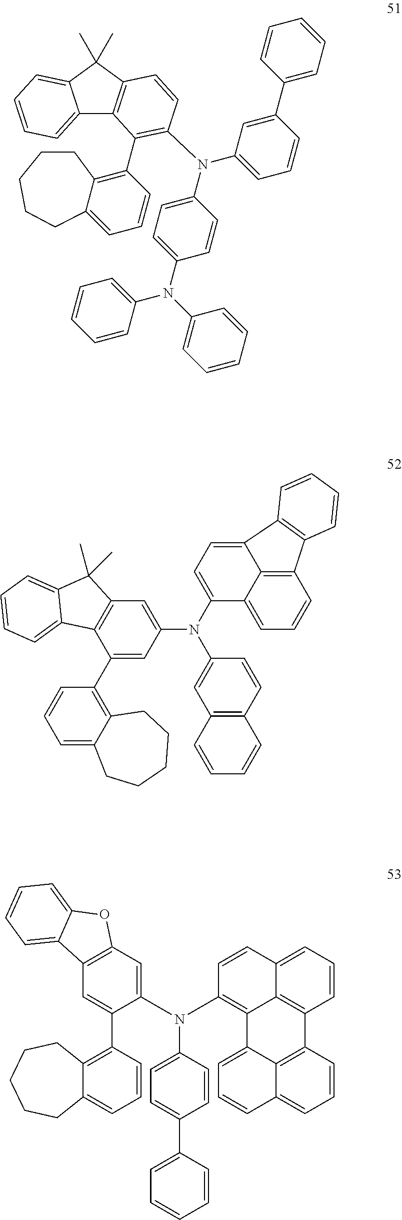 [SMILES: CC1(C)c2ccccc2-c2c1ccc(N(c1ccc(N(c3ccccc3)c3ccccc3)cc1)c1cccc(-c3ccccc3)c1)c2-c1cccc2c1CCCCC2 ; CC1(C)c2ccccc2-c2c(-c3cccc4c3CCCCC4)cc(N(c3ccc4ccccc4c3)c3ccc4c5c(cccc35)-c3ccccc3-4)cc21 ; c1ccc(-c2ccc(N(c3cc4oc5ccccc5c4cc3-c3cccc4c3CCCCC4)c3ccc4cccc5c6cccc7cccc(c3c45)c76)cc2)cc1]

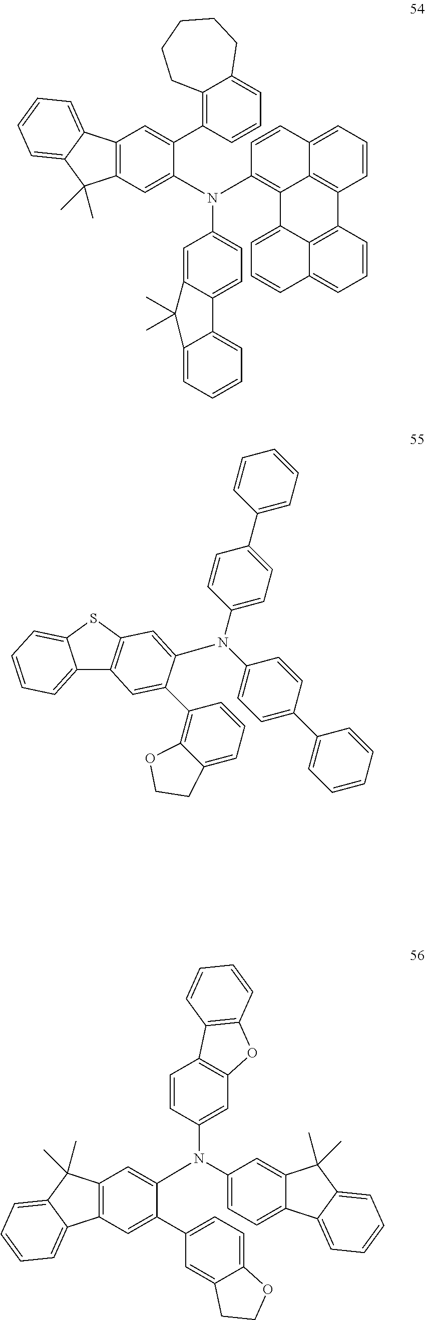 [SMILES: CC1(C)c2ccccc2-c2ccc(N(c3cc4c(cc3-c3cccc5c3CCCCC5)-c3ccccc3C4(C)C)c3ccc4cccc5c6cccc7cccc(c3c45)c76)cc21 ; c1ccc(-c2ccc(N(c3ccc(-c4ccccc4)cc3)c3cc4sc5ccccc5c4cc3-c3cccc4c3OCC4)cc2)cc1 ; CC1(C)c2ccccc2-c2ccc(N(c3ccc4c(c3)oc3ccccc34)c3cc4c(cc3-c3ccc5c(c3)CCO5)-c3ccccc3C4(C)C)cc21]

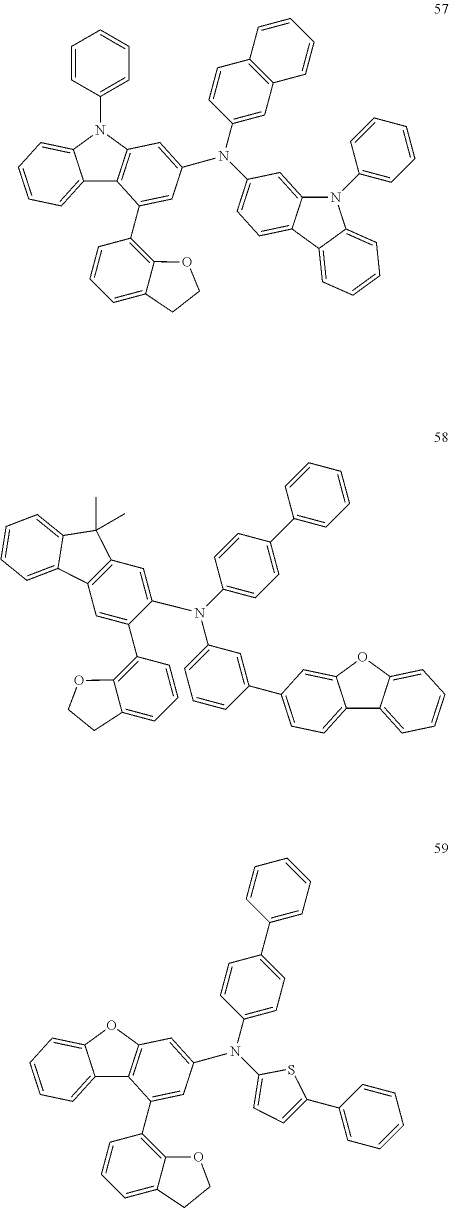 [SMILES: c1ccc(-n2c3ccccc3c3ccc(N(c4ccc5ccccc5c4)c4cc(-c5cccc6c5OCC6)c5c6ccccc6n(-c6ccccc6)c5c4)cc32)cc1 ; CC1(C)c2ccccc2-c2cc(-c3cccc4c3OCC4)c(N(c3ccc(-c4ccccc4)cc3)c3cccc(-c4ccc5c(c4)oc4ccccc45)c3)cc21 ; c1ccc(-c2ccc(N(c3cc(-c4cccc5c4OCC5)c4c(c3)oc3ccccc34)c3ccc(-c4ccccc4)s3)cc2)cc1]

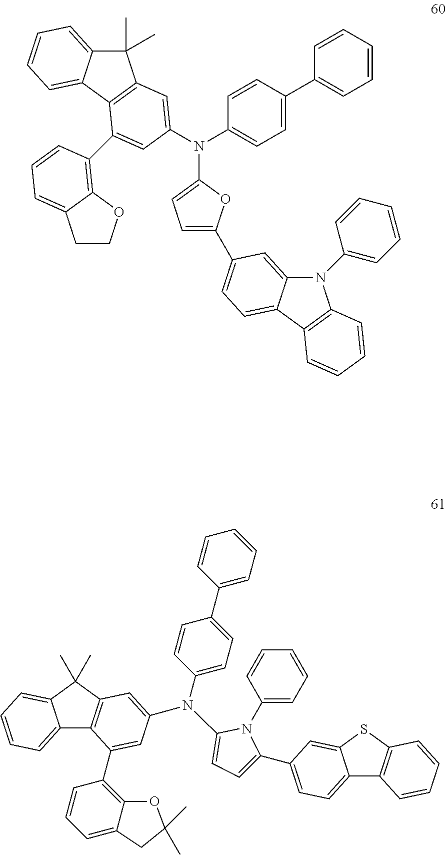 [SMILES: CC1(C)c2ccccc2-c2c(-c3cccc4c3OCC4)cc(N(c3ccc(-c4ccccc4)cc3)c3ccc(-c4ccc5c6ccccc6n(-c6ccccc6)c5c4)o3)cc21 ; CC1(C)Cc2cccc(-c3cc(N(c4ccc(-c5ccccc5)cc4)c4ccc(-c5ccc6c(c5)sc5ccccc56)n4-c4ccccc4)cc4c3-c3ccccc3C4(C)C)c2O1]

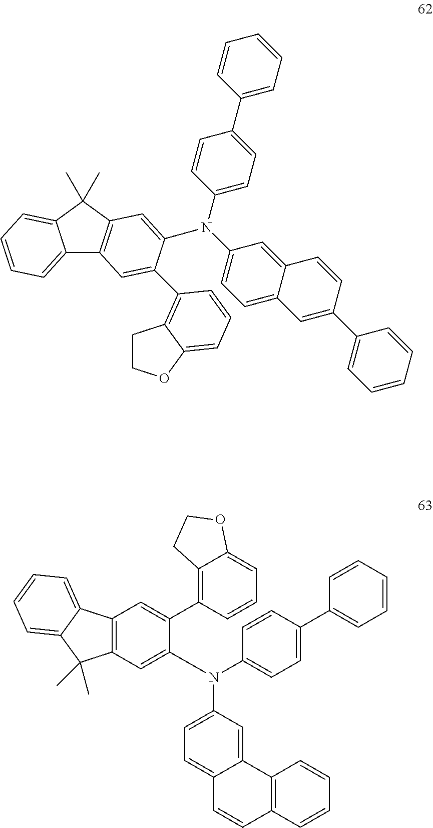 [SMILES: CC1(C)c2ccccc2-c2cc(-c3cccc4c3CCO4)c(N(c3ccc(-c4ccccc4)cc3)c3ccc4cc(-c5ccccc5)ccc4c3)cc21 ; CC1(C)c2ccccc2-c2cc(-c3cccc4c3CCO4)c(N(c3ccc(-c4ccccc4)cc3)c3ccc4ccc5ccccc5c4c3)cc21]

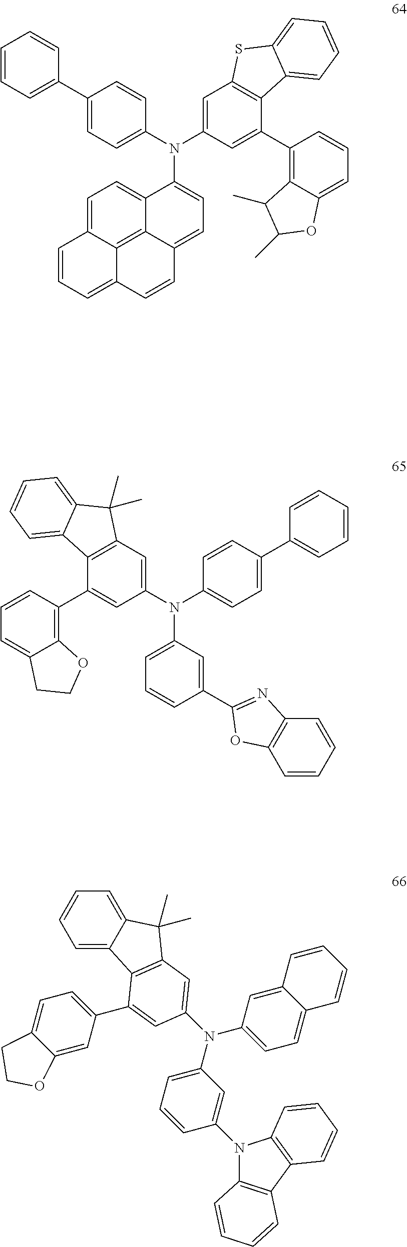 [SMILES: CC1Oc2cccc(-c3cc(N(c4ccc(-c5ccccc5)cc4)c4ccc5ccc6cccc7ccc4c5c67)cc4sc5ccccc5c34)c2C1C ; CC1(C)c2ccccc2-c2c(-c3cccc4c3OCC4)cc(N(c3ccc(-c4ccccc4)cc3)c3cccc(-c4nc5ccccc5o4)c3)cc21 ; CC1(C)c2ccccc2-c2c(-c3ccc4c(c3)OCC4)cc(N(c3cccc(-n4c5ccccc5c5ccccc54)c3)c3ccc4ccccc4c3)cc21]

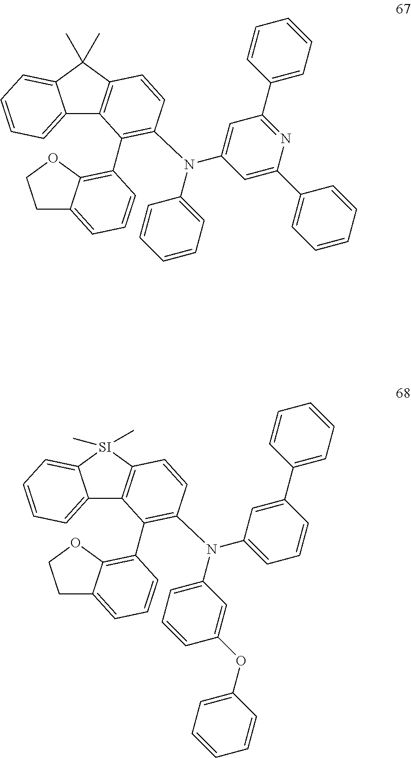

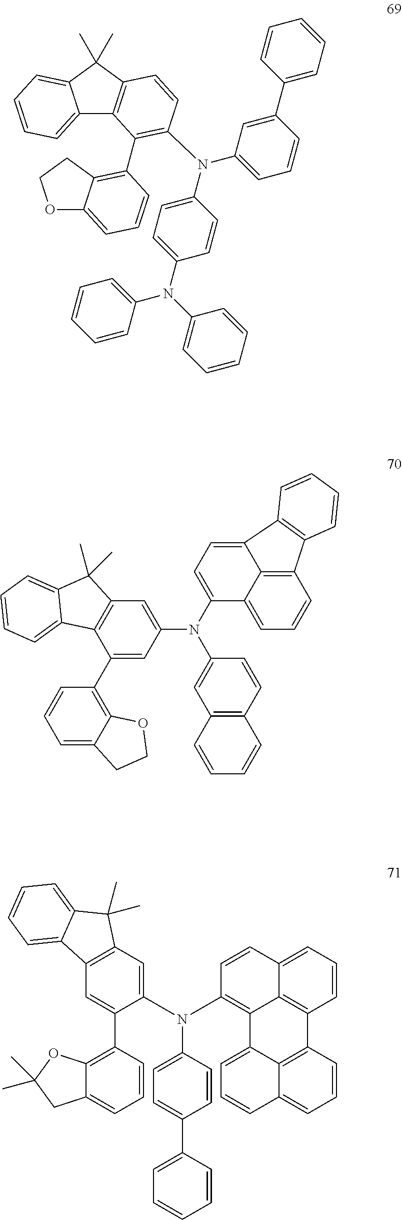 [SMILES: CC1(C)c2ccccc2-c2c1ccc(N(c1ccc(N(c3ccccc3)c3ccccc3)cc1)c1cccc(-c3ccccc3)c1)c2-c1cccc2c1CCO2 ; CC1(C)c2ccccc2-c2c(-c3cccc4c3OCC4)cc(N(c3ccc4ccccc4c3)c3ccc4c5c(cccc35)-c3ccccc3-4)cc21 ; CC1(C)Cc2cccc(-c3cc4c(cc3N(c3ccc(-c5ccccc5)cc3)c3ccc5cccc6c7cccc8cccc(c3c56)c87)C(C)(C)c3ccccc3-4)c2O1]

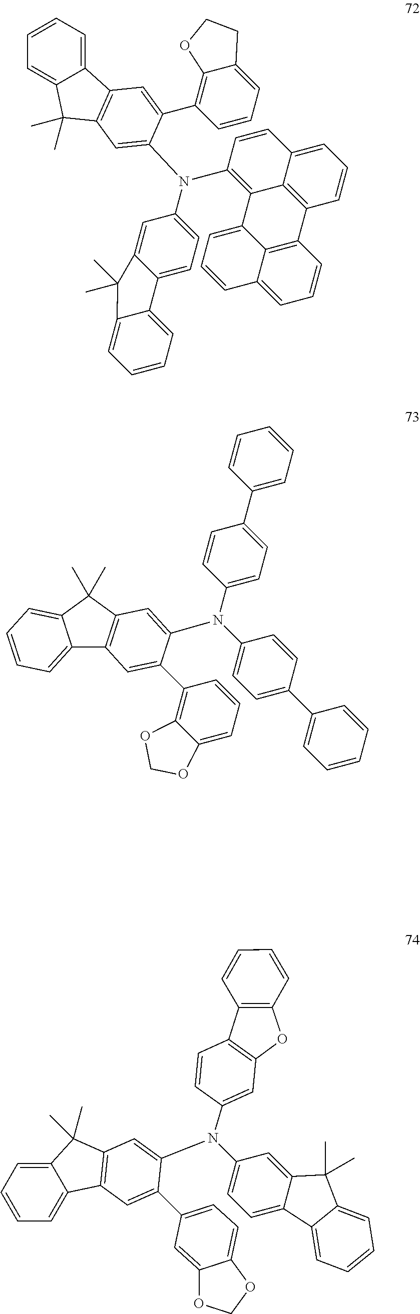 [SMILES: CC1(C)c2ccccc2-c2ccc(N(c3cc4c(cc3-c3cccc5c3OCC5)-c3ccccc3C4(C)C)c3ccc4cccc5c6cccc7cccc(c3c45)c76)cc21 ; CC1(C)c2ccccc2-c2cc(-c3cccc4c3OCO4)c(N(c3ccc(-c4ccccc4)cc3)c3ccc(-c4ccccc4)cc3)cc21 ; CC1(C)c2ccccc2-c2ccc(N(c3ccc4c(c3)oc3ccccc34)c3cc4c(cc3-c3ccc5c(c3)OCO5)-c3ccccc3C4(C)C)cc21]

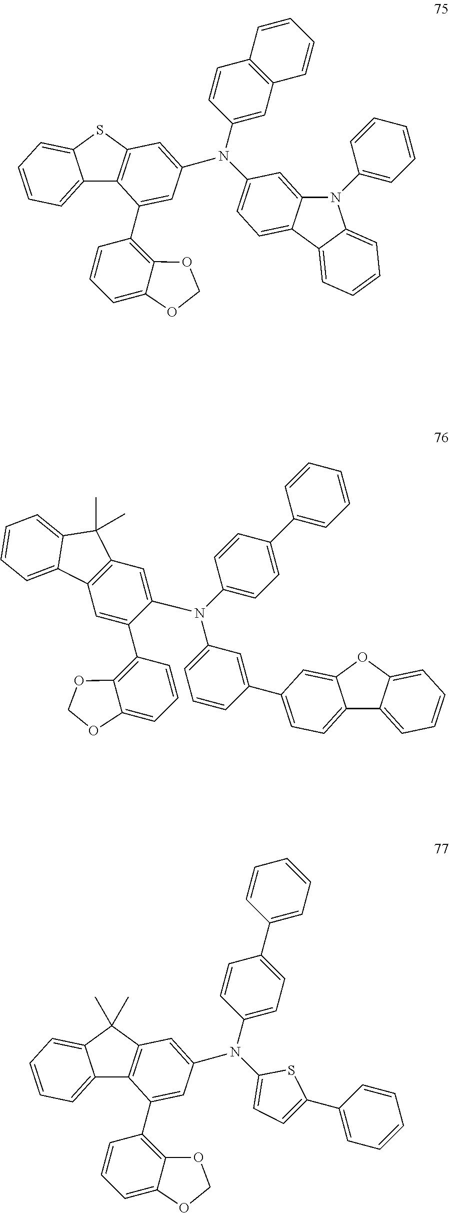 [SMILES: c1ccc(-n2c3ccccc3c3ccc(N(c4ccc5ccccc5c4)c4cc(-c5cccc6c5OCO6)c5c(c4)sc4ccccc45)cc32)cc1 ; CC1(C)c2ccccc2-c2cc(-c3cccc4c3OCO4)c(N(c3ccc(-c4ccccc4)cc3)c3cccc(-c4ccc5c(c4)oc4ccccc45)c3)cc21 ; CC1(C)c2ccccc2-c2c(-c3cccc4c3OCO4)cc(N(c3ccc(-c4ccccc4)cc3)c3ccc(-c4ccccc4)s3)cc21]

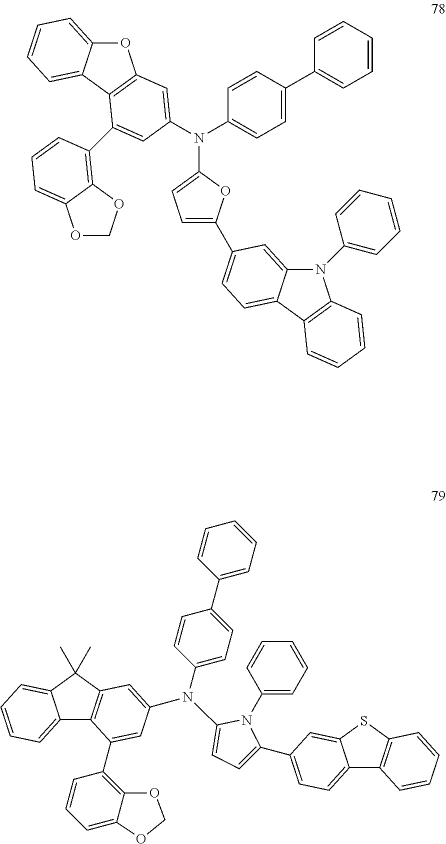 [SMILES: c1ccc(-c2ccc(N(c3cc(-c4cccc5c4OCO5)c4c(c3)oc3ccccc34)c3ccc(-c4ccc5c6ccccc6n(-c6ccccc6)c5c4)o3)cc2)cc1 ; CC1(C)c2ccccc2-c2c(-c3cccc4c3OCO4)cc(N(c3ccc(-c4ccccc4)cc3)c3ccc(-c4ccc5c(c4)sc4ccccc45)n3-c3ccccc3)cc21]

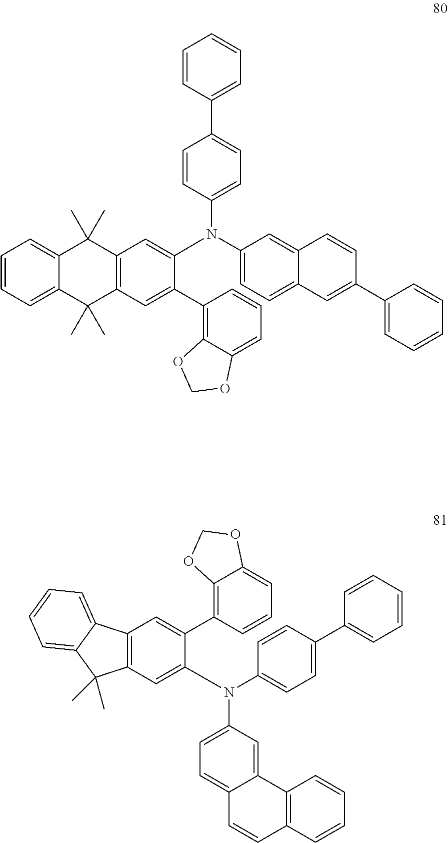 [SMILES: CC1(C)c2ccccc2C(C)(C)c2cc(N(c3ccc(-c4ccccc4)cc3)c3ccc4cc(-c5ccccc5)ccc4c3)c(-c3cccc4c3OCO4)cc21 ; CC1(C)c2ccccc2-c2cc(-c3cccc4c3OCO4)c(N(c3ccc(-c4ccccc4)cc3)c3ccc4ccc5ccccc5c4c3)cc21]

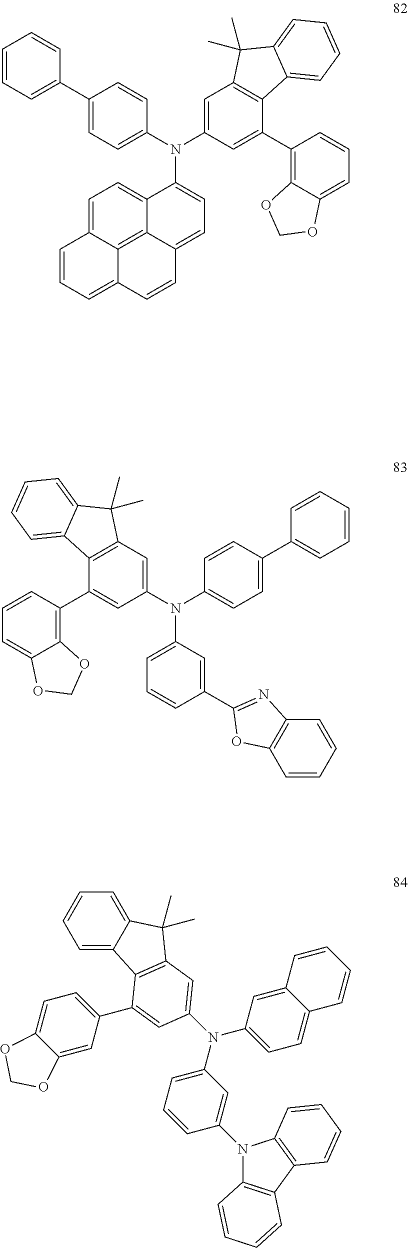 [SMILES: CC1(C)c2ccccc2-c2c(-c3cccc4c3OCO4)cc(N(c3ccc(-c4ccccc4)cc3)c3ccc4ccc5cccc6ccc3c4c56)cc21 ; CC1(C)c2ccccc2-c2c(-c3cccc4c3OCO4)cc(N(c3ccc(-c4ccccc4)cc3)c3cccc(-c4nc5ccccc5o4)c3)cc21 ; CC1(C)c2ccccc2-c2c(-c3ccc4c(c3)OCO4)cc(N(c3cccc(-n4c5ccccc5c5ccccc54)c3)c3ccc4ccccc4c3)cc21]

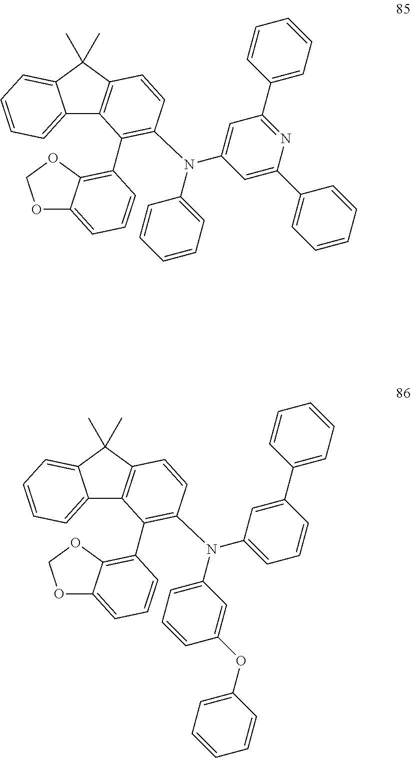 [SMILES: CC1(C)c2ccccc2-c2c1ccc(N(c1ccccc1)c1cc(-c3ccccc3)nc(-c3ccccc3)c1)c2-c1cccc2c1OCO2 ; CC1(C)c2ccccc2-c2c1ccc(N(c1cccc(Oc3ccccc3)c1)c1cccc(-c3ccccc3)c1)c2-c1cccc2c1OCO2]

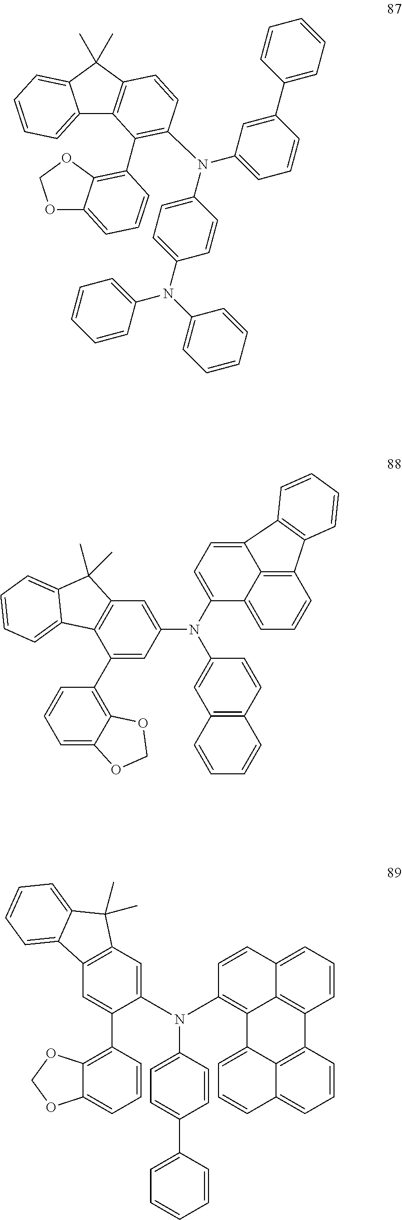 [SMILES: CC1(C)c2ccccc2-c2c1ccc(N(c1ccc(N(c3ccccc3)c3ccccc3)cc1)c1cccc(-c3ccccc3)c1)c2-c1cccc2c1OCO2 ; CC1(C)c2ccccc2-c2c(-c3cccc4c3OCO4)cc(N(c3ccc4ccccc4c3)c3ccc4c5c(cccc35)-c3ccccc3-4)cc21 ; CC1(C)c2ccccc2-c2cc(-c3cccc4c3OCO4)c(N(c3ccc(-c4ccccc4)cc3)c3ccc4cccc5c6cccc7cccc(c3c45)c76)cc21]

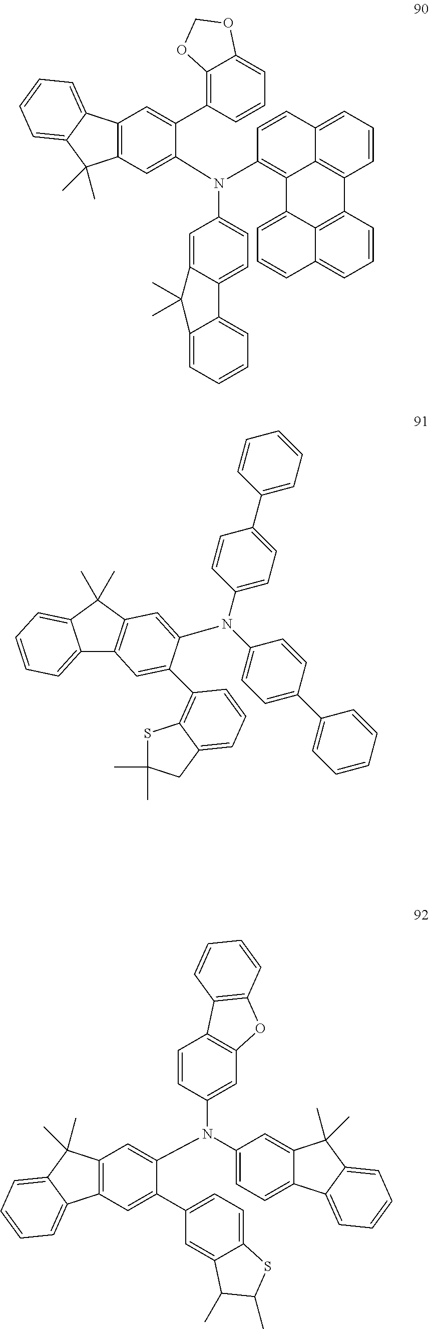 [SMILES: CC1(C)c2ccccc2-c2ccc(N(c3cc4c(cc3-c3cccc5c3OCO5)-c3ccccc3C4(C)C)c3ccc4cccc5c6cccc7cccc(c3c45)c76)cc21 ; CC1(C)Cc2cccc(-c3cc4c(cc3N(c3ccc(-c5ccccc5)cc3)c3ccc(-c5ccccc5)cc3)C(C)(C)c3ccccc3-4)c2S1 ; CC1Sc2ccc(-c3cc4c(cc3N(c3ccc5c(c3)C(C)(C)c3ccccc3-5)c3ccc5c(c3)oc3ccccc35)C(C)(C)c3ccccc3-4)cc2C1C]

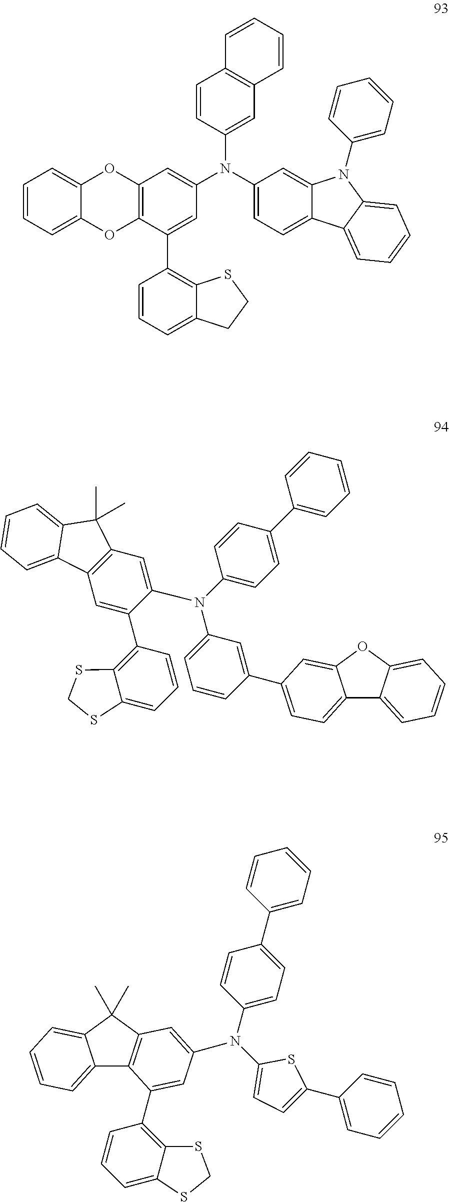 [SMILES: c1ccc(-n2c3ccccc3c3ccc(N(c4cc5c(c(-c6cccc7c6SCC7)c4)Oc4ccccc4O5)c4ccc5ccccc5c4)cc32)cc1 ; CC1(C)c2ccccc2-c2cc(-c3cccc4c3SCS4)c(N(c3ccc(-c4ccccc4)cc3)c3cccc(-c4ccc5c(c4)oc4ccccc45)c3)cc21 ; CC1(C)c2ccccc2-c2c(-c3cccc4c3SCS4)cc(N(c3ccc(-c4ccccc4)cc3)c3ccc(-c4ccccc4)s3)cc21]

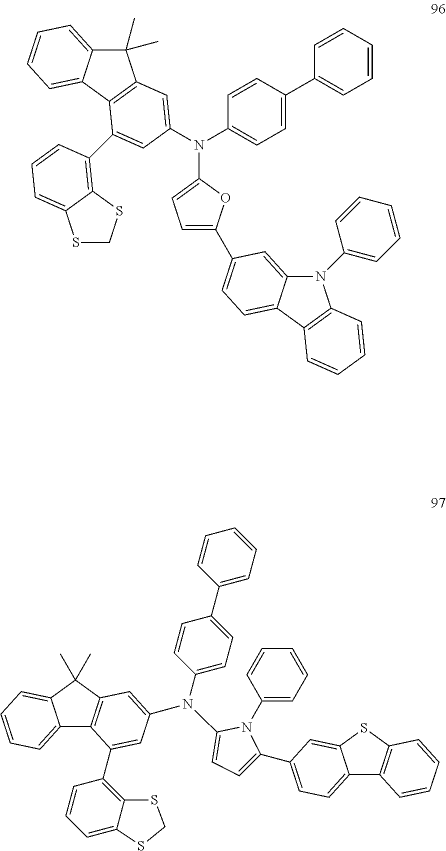 [SMILES: CC1(C)c2ccccc2-c2c(-c3cccc4c3SCS4)cc(N(c3ccc(-c4ccccc4)cc3)c3ccc(-c4ccc5c6ccccc6n(-c6ccccc6)c5c4)o3)cc21 ; CC1(C)c2ccccc2-c2c(-c3cccc4c3SCS4)cc(N(c3ccc(-c4ccccc4)cc3)c3ccc(-c4ccc5c(c4)sc4ccccc45)n3-c3ccccc3)cc21]

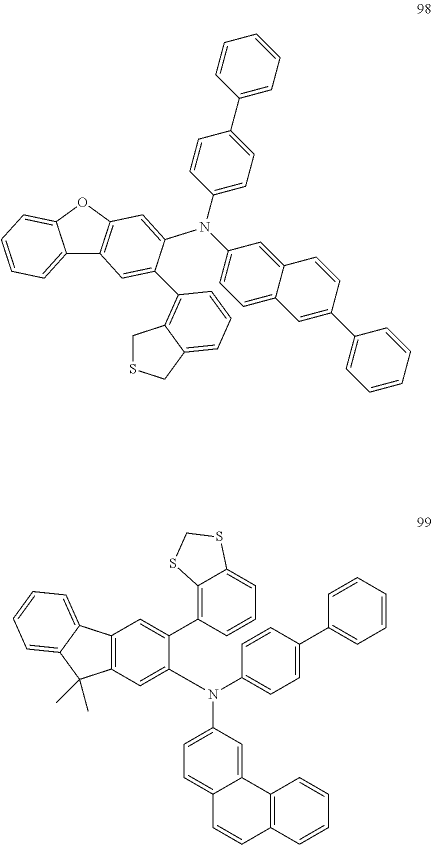 [SMILES: c1ccc(-c2ccc(N(c3ccc4cc(-c5ccccc5)ccc4c3)c3cc4oc5ccccc5c4cc3-c3cccc4c3CSC4)cc2)cc1 ; CC1(C)c2ccccc2-c2cc(-c3cccc4c3SCS4)c(N(c3ccc(-c4ccccc4)cc3)c3ccc4ccc5ccccc5c4c3)cc21]

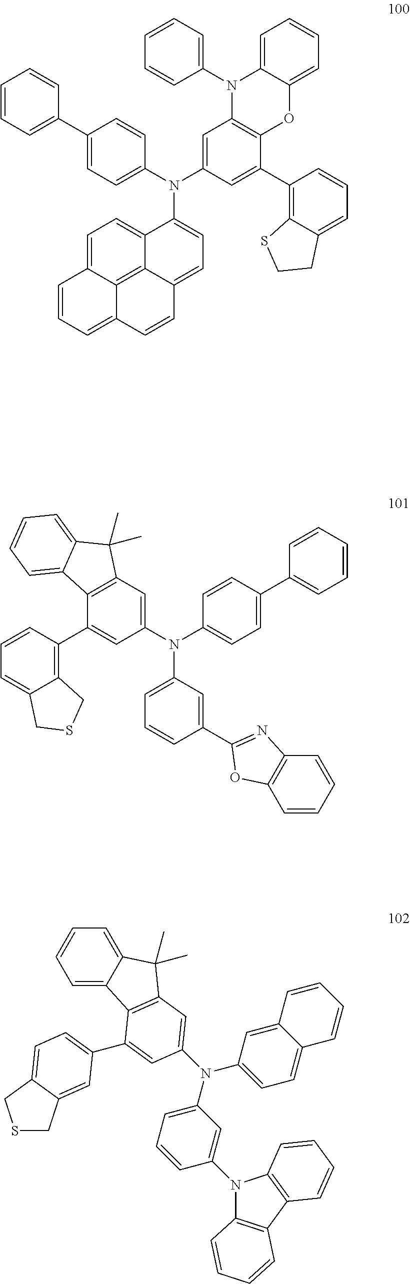 [SMILES: c1ccc(-c2ccc(N(c3cc(-c4cccc5c4SCC5)c4c(c3)N(c3ccccc3)c3ccccc3O4)c3ccc4ccc5cccc6ccc3c4c56)cc2)cc1 ; CC1(C)c2ccccc2-c2c(-c3cccc4c3CSC4)cc(N(c3ccc(-c4ccccc4)cc3)c3cccc(-c4nc5ccccc5o4)c3)cc21 ; CC1(C)c2ccccc2-c2c(-c3ccc4c(c3)CSC4)cc(N(c3cccc(-n4c5ccccc5c5ccccc54)c3)c3ccc4ccccc4c3)cc21]

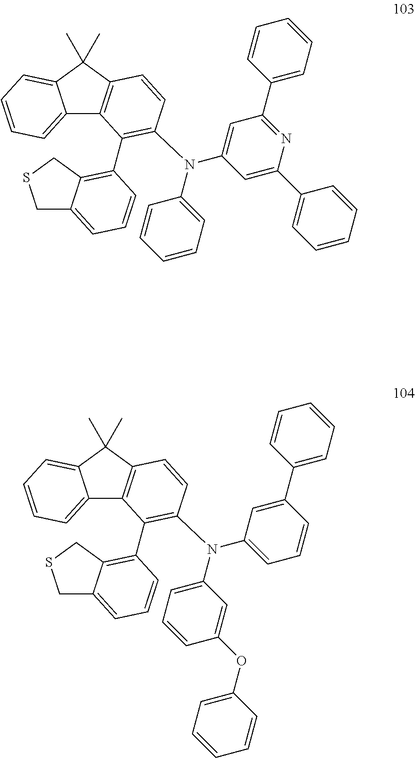 [SMILES: CC1(C)c2ccccc2-c2c1ccc(N(c1ccccc1)c1cc(-c3ccccc3)nc(-c3ccccc3)c1)c2-c1cccc2c1CSC2 ; CC1(C)c2ccccc2-c2c1ccc(N(c1cccc(Oc3ccccc3)c1)c1cccc(-c3ccccc3)c1)c2-c1cccc2c1CSC2]

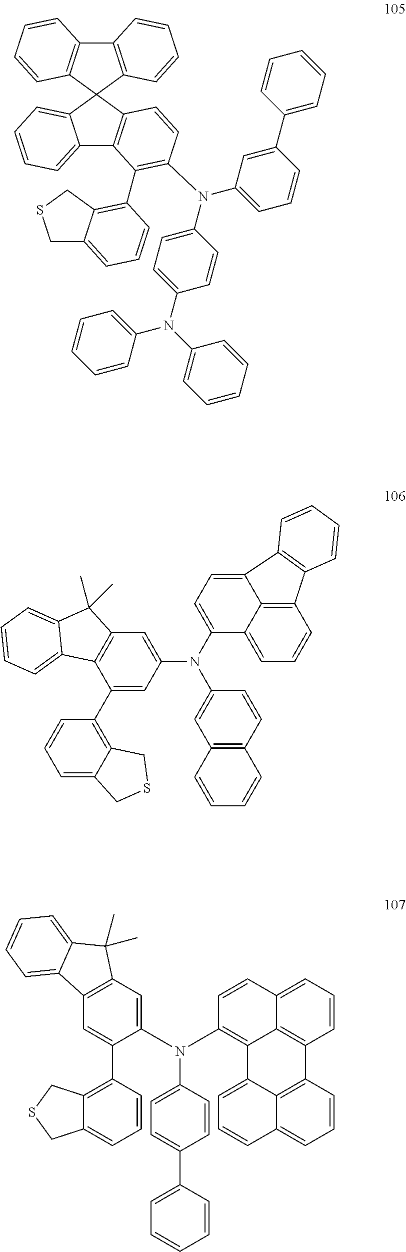 [SMILES: c1ccc(-c2cccc(N(c3ccc(N(c4ccccc4)c4ccccc4)cc3)c3ccc4c(c3-c3cccc5c3CSC5)-c3ccccc3C43c4ccccc4-c4ccccc43)c2)cc1 ; CC1(C)c2ccccc2-c2c(-c3cccc4c3CSC4)cc(N(c3ccc4ccccc4c3)c3ccc4c5c(cccc35)-c3ccccc3-4)cc21 ; CC1(C)c2ccccc2-c2cc(-c3cccc4c3CSC4)c(N(c3ccc(-c4ccccc4)cc3)c3ccc4cccc5c6cccc7cccc(c3c45)c76)cc21]

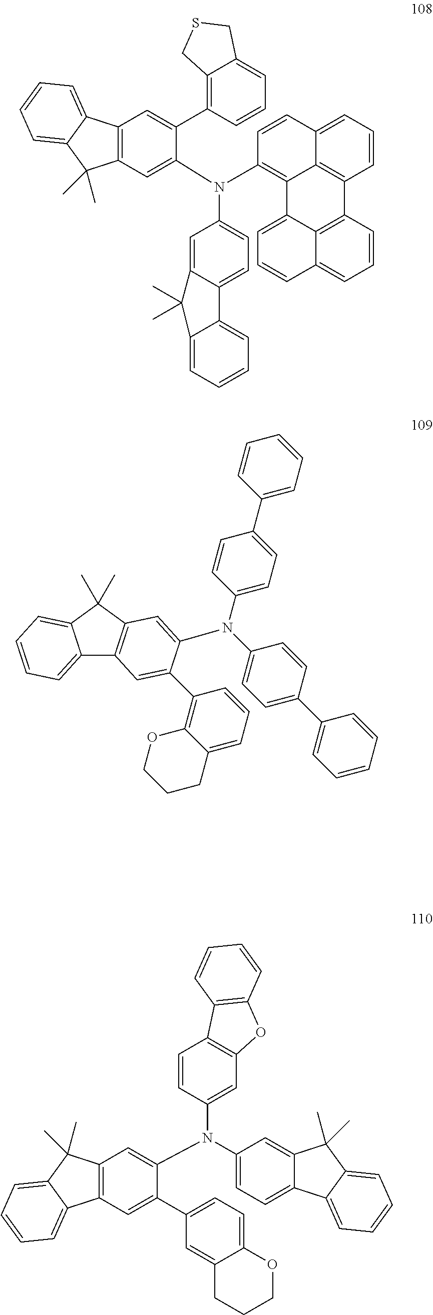 [SMILES: CC1(C)c2ccccc2-c2ccc(N(c3cc4c(cc3-c3cccc5c3CSC5)-c3ccccc3C4(C)C)c3ccc4cccc5c6cccc7cccc(c3c45)c76)cc21 ; CC1(C)c2ccccc2-c2cc(-c3cccc4c3OCCC4)c(N(c3ccc(-c4ccccc4)cc3)c3ccc(-c4ccccc4)cc3)cc21 ; CC1(C)c2ccccc2-c2ccc(N(c3ccc4c(c3)oc3ccccc34)c3cc4c(cc3-c3ccc5c(c3)CCCO5)-c3ccccc3C4(C)C)cc21]

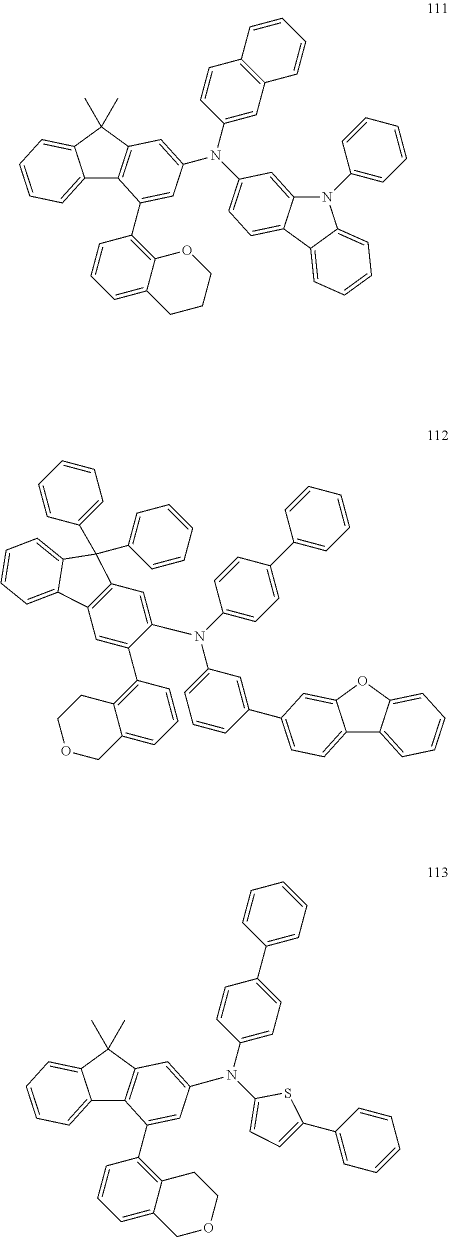 [SMILES: CC1(C)c2ccccc2-c2c(-c3cccc4c3OCCC4)cc(N(c3ccc4ccccc4c3)c3ccc4c5ccccc5n(-c5ccccc5)c4c3)cc21 ; c1ccc(-c2ccc(N(c3cccc(-c4ccc5c(c4)oc4ccccc45)c3)c3cc4c(cc3-c3cccc5c3CCOC5)-c3ccccc3C4(c3ccccc3)c3ccccc3)cc2)cc1 ; CC1(C)c2ccccc2-c2c(-c3cccc4c3CCOC4)cc(N(c3ccc(-c4ccccc4)cc3)c3ccc(-c4ccccc4)s3)cc21]

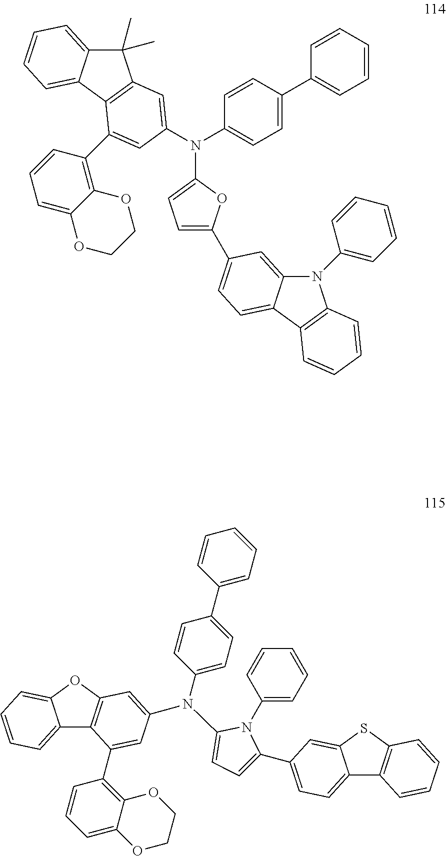 [SMILES: CC1(C)c2ccccc2-c2c(-c3cccc4c3OCCO4)cc(N(c3ccc(-c4ccccc4)cc3)c3ccc(-c4ccc5c6ccccc6n(-c6ccccc6)c5c4)o3)cc21 ; c1ccc(-c2ccc(N(c3cc(-c4cccc5c4OCCO5)c4c(c3)oc3ccccc34)c3ccc(-c4ccc5c(c4)sc4ccccc45)n3-c3ccccc3)cc2)cc1]

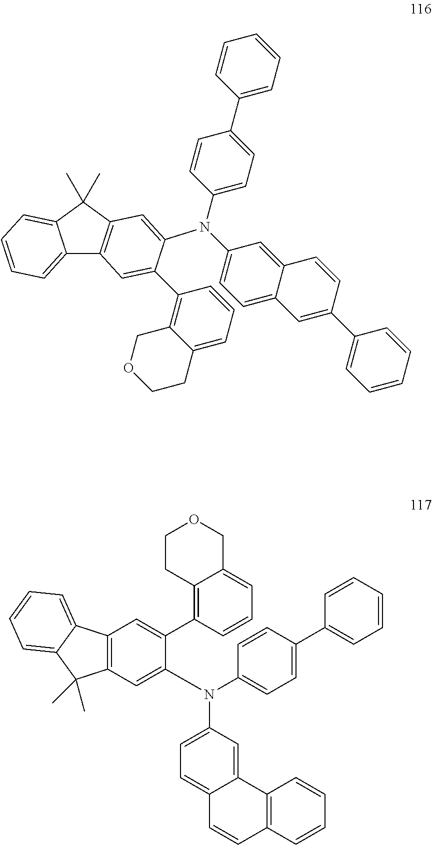 [SMILES: CC1(C)c2ccccc2-c2cc(-c3cccc4c3COCC4)c(N(c3ccc(-c4ccccc4)cc3)c3ccc4cc(-c5ccccc5)ccc4c3)cc21 ; CC1(C)c2ccccc2-c2cc(-c3cccc4c3CCOC4)c(N(c3ccc(-c4ccccc4)cc3)c3ccc4ccc5ccccc5c4c3)cc21]

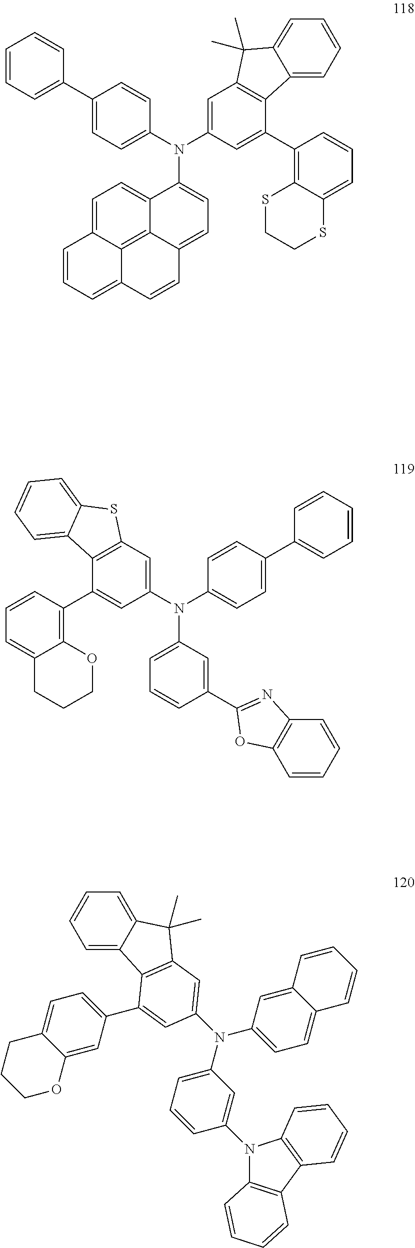 [SMILES: CC1(C)c2ccccc2-c2c(-c3cccc4c3SCCS4)cc(N(c3ccc(-c4ccccc4)cc3)c3ccc4ccc5cccc6ccc3c4c56)cc21 ; c1ccc(-c2ccc(N(c3cccc(-c4nc5ccccc5o4)c3)c3cc(-c4cccc5c4OCCC5)c4c(c3)sc3ccccc34)cc2)cc1 ; CC1(C)c2ccccc2-c2c(-c3ccc4c(c3)OCCC4)cc(N(c3cccc(-n4c5ccccc5c5ccccc54)c3)c3ccc4ccccc4c3)cc21]

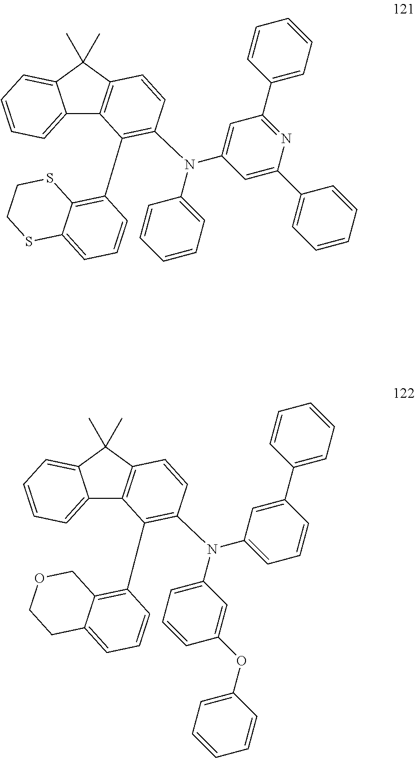 [SMILES: CC1(C)c2ccccc2-c2c1ccc(N(c1ccccc1)c1cc(-c3ccccc3)nc(-c3ccccc3)c1)c2-c1cccc2c1SCCS2 ; CC1(C)c2ccccc2-c2c1ccc(N(c1cccc(Oc3ccccc3)c1)c1cccc(-c3ccccc3)c1)c2-c1cccc2c1COCC2]

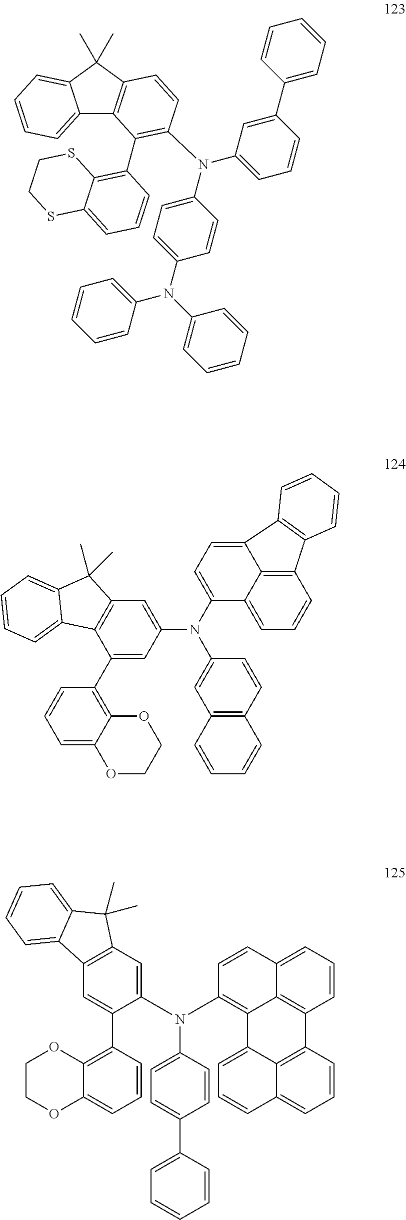 [SMILES: CC1(C)c2ccccc2-c2c1ccc(N(c1ccc(N(c3ccccc3)c3ccccc3)cc1)c1cccc(-c3ccccc3)c1)c2-c1cccc2c1SCCS2 ; CC1(C)c2ccccc2-c2c(-c3cccc4c3OCCO4)cc(N(c3ccc4ccccc4c3)c3ccc4c5c(cccc35)-c3ccccc3-4)cc21 ; CC1(C)c2ccccc2-c2cc(-c3cccc4c3OCCO4)c(N(c3ccc(-c4ccccc4)cc3)c3ccc4cccc5c6cccc7cccc(c3c45)c76)cc21]

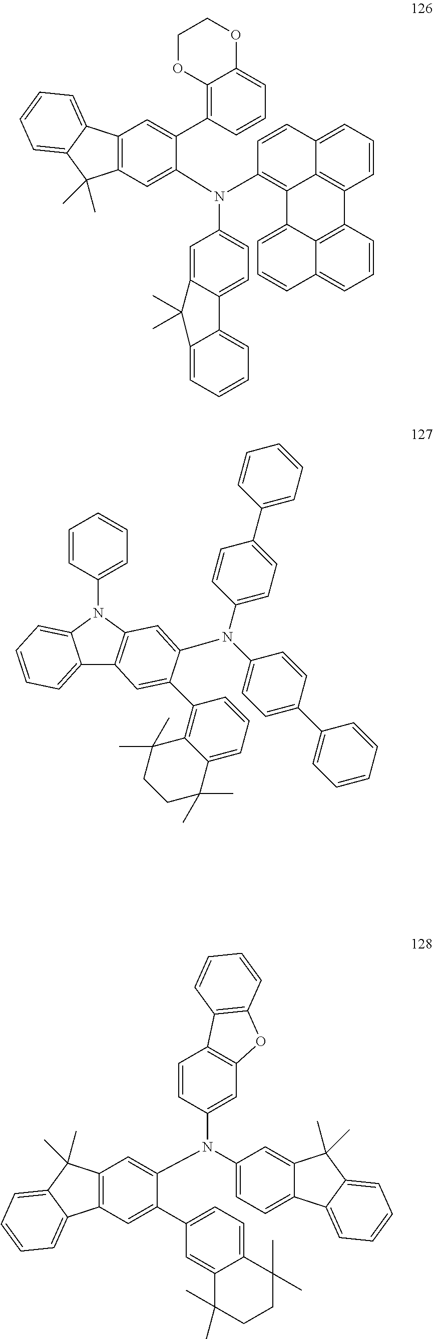 [SMILES: CC1(C)c2ccccc2-c2ccc(N(c3cc4c(cc3-c3cccc5c3OCCO5)-c3ccccc3C4(C)C)c3ccc4cccc5c6cccc7cccc(c3c45)c76)cc21 ; CC1(C)CCC(C)(C)c2c(-c3cc4c5ccccc5n(-c5ccccc5)c4cc3N(c3ccc(-c4ccccc4)cc3)c3ccc(-c4ccccc4)cc3)cccc21 ; CC1(C)CCC(C)(C)c2cc(-c3cc4c(cc3N(c3ccc5c(c3)C(C)(C)c3ccccc3-5)c3ccc5c(c3)oc3ccccc35)C(C)(C)c3ccccc3-4)ccc21]

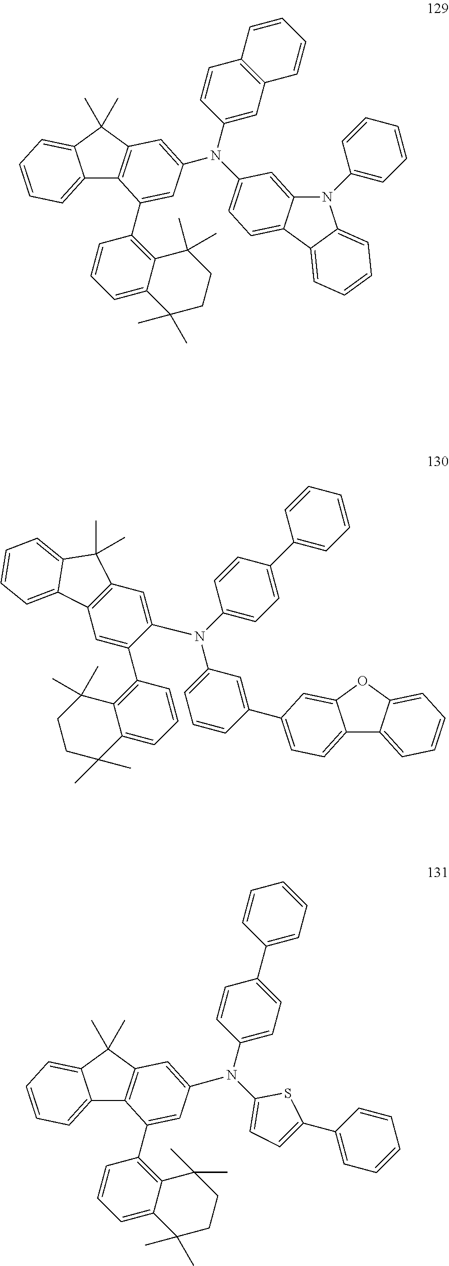 [SMILES: CC1(C)CCC(C)(C)c2c(-c3cc(N(c4ccc5ccccc5c4)c4ccc5c6ccccc6n(-c6ccccc6)c5c4)cc4c3-c3ccccc3C4(C)C)cccc21 ; CC1(C)CCC(C)(C)c2c(-c3cc4c(cc3N(c3ccc(-c5ccccc5)cc3)c3cccc(-c5ccc6c(c5)oc5ccccc56)c3)C(C)(C)c3ccccc3-4)cccc21 ; CC1(C)CCC(C)(C)c2c(-c3cc(N(c4ccc(-c5ccccc5)cc4)c4ccc(-c5ccccc5)s4)cc4c3-c3ccccc3C4(C)C)cccc21]

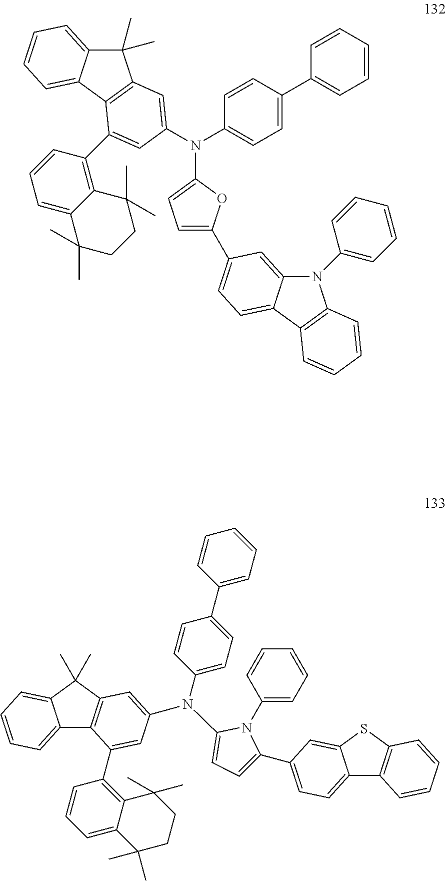 [SMILES: CC1(C)CCC(C)(C)c2c(-c3cc(N(c4ccc(-c5ccccc5)cc4)c4ccc(-c5ccc6c7ccccc7n(-c7ccccc7)c6c5)o4)cc4c3-c3ccccc3C4(C)C)cccc21 ; CC1(C)CCC(C)(C)c2c(-c3cc(N(c4ccc(-c5ccccc5)cc4)c4ccc(-c5ccc6c(c5)sc5ccccc56)n4-c4ccccc4)cc4c3-c3ccccc3C4(C)C)cccc21]

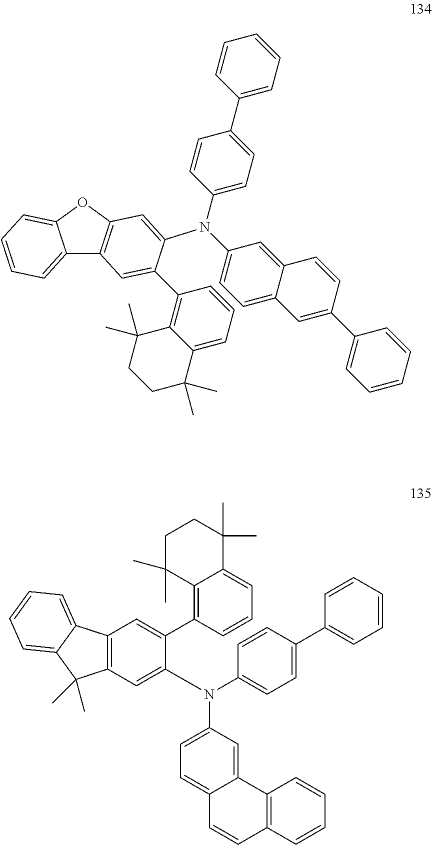 [SMILES: CC1(C)CCC(C)(C)c2c(-c3cc4c(cc3N(c3ccc(-c5ccccc5)cc3)c3ccc5cc(-c6ccccc6)ccc5c3)oc3ccccc34)cccc21 ; CC1(C)CCC(C)(C)c2c(-c3cc4c(cc3N(c3ccc(-c5ccccc5)cc3)c3ccc5ccc6ccccc6c5c3)C(C)(C)c3ccccc3-4)cccc21]

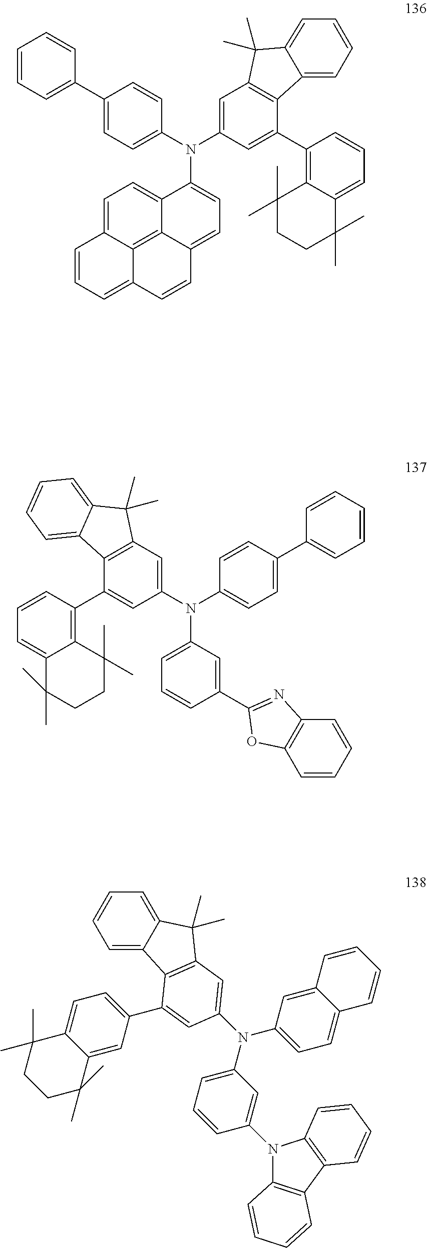

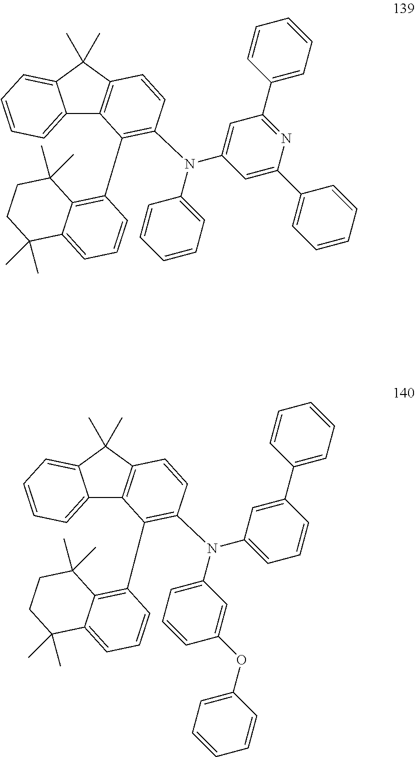 [SMILES: CC1(C)CCC(C)(C)c2c(-c3c(N(c4ccccc4)c4cc(-c5ccccc5)nc(-c5ccccc5)c4)ccc4c3-c3ccccc3C4(C)C)cccc21 ; CC1(C)CCC(C)(C)c2c(-c3c(N(c4cccc(Oc5ccccc5)c4)c4cccc(-c5ccccc5)c4)ccc4c3-c3ccccc3C4(C)C)cccc21]

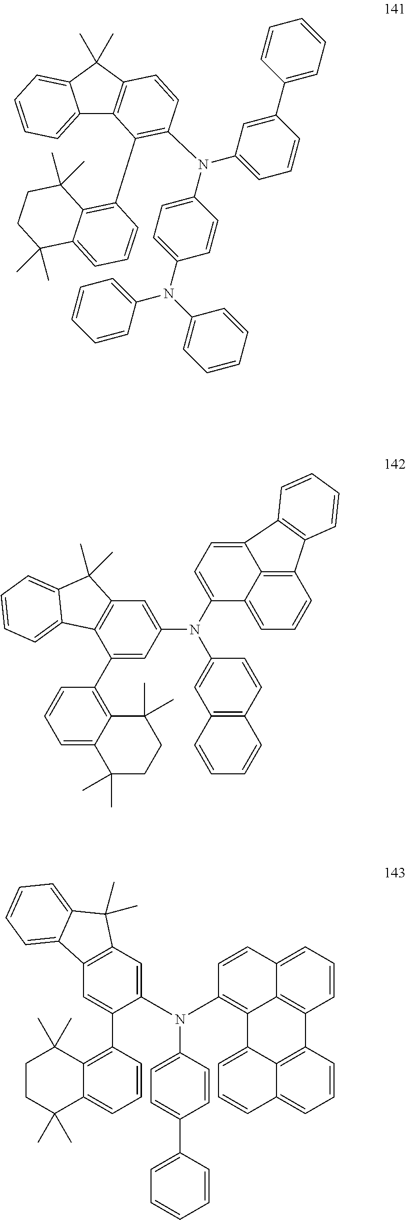 [SMILES: CC1(C)CCC(C)(C)c2c(-c3c(N(c4ccc(N(c5ccccc5)c5ccccc5)cc4)c4cccc(-c5ccccc5)c4)ccc4c3-c3ccccc3C4(C)C)cccc21 ; CC1(C)CCC(C)(C)c2c(-c3cc(N(c4ccc5ccccc5c4)c4ccc5c6c(cccc46)-c4ccccc4-5)cc4c3-c3ccccc3C4(C)C)cccc21 ; CC1(C)CCC(C)(C)c2c(-c3cc4c(cc3N(c3ccc(-c5ccccc5)cc3)c3ccc5cccc6c7cccc8cccc(c3c56)c87)C(C)(C)c3ccccc3-4)cccc21]

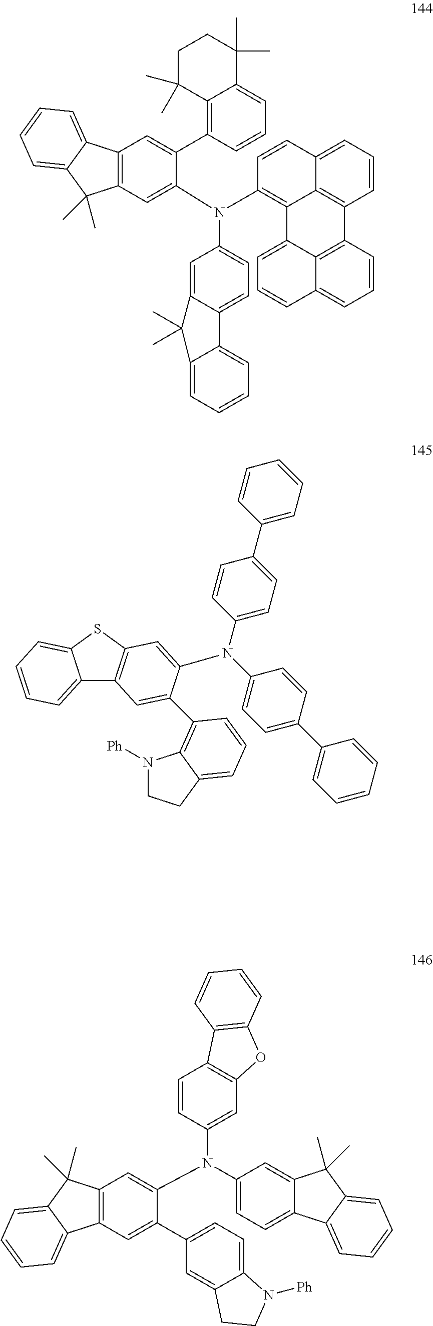 [SMILES: CC1(C)CCC(C)(C)c2c(-c3cc4c(cc3N(c3ccc5c(c3)C(C)(C)c3ccccc3-5)c3ccc5cccc6c7cccc8cccc(c3c56)c87)C(C)(C)c3ccccc3-4)cccc21 ; c1ccc(-c2ccc(N(c3ccc(-c4ccccc4)cc3)c3cc4sc5ccccc5c4cc3-c3cccc4c3N(c3ccccc3)CC4)cc2)cc1 ; CC1(C)c2ccccc2-c2ccc(N(c3ccc4c(c3)oc3ccccc34)c3cc4c(cc3-c3ccc5c(c3)CCN5c3ccccc3)-c3ccccc3C4(C)C)cc21]

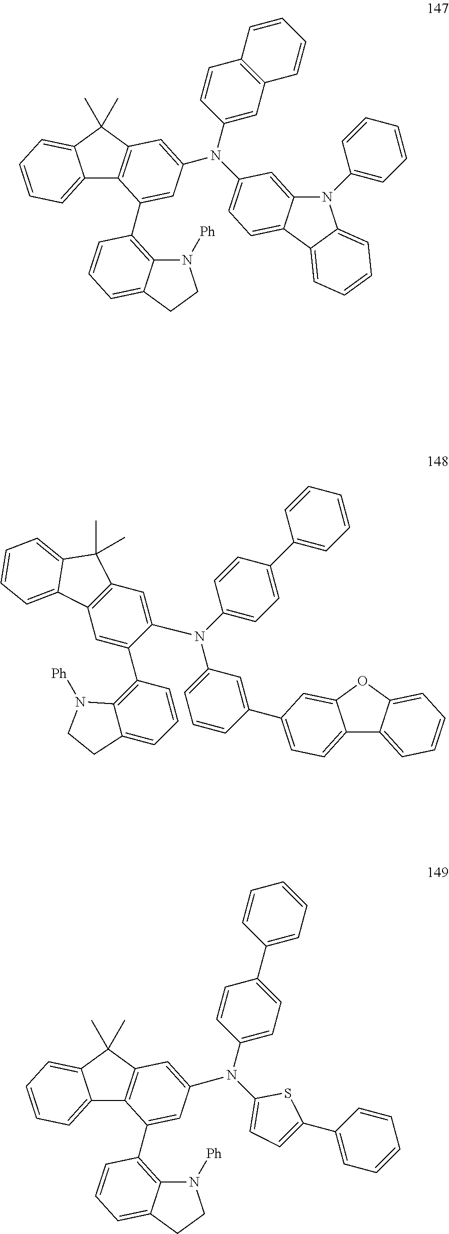 [SMILES: CC1(C)c2ccccc2-c2c(-c3cccc4c3N(c3ccccc3)CC4)cc(N(c3ccc4ccccc4c3)c3ccc4c5ccccc5n(-c5ccccc5)c4c3)cc21 ; CC1(C)c2ccccc2-c2cc(-c3cccc4c3N(c3ccccc3)CC4)c(N(c3ccc(-c4ccccc4)cc3)c3cccc(-c4ccc5c(c4)oc4ccccc45)c3)cc21 ; CC1(C)c2ccccc2-c2c(-c3cccc4c3N(c3ccccc3)CC4)cc(N(c3ccc(-c4ccccc4)cc3)c3ccc(-c4ccccc4)s3)cc21]

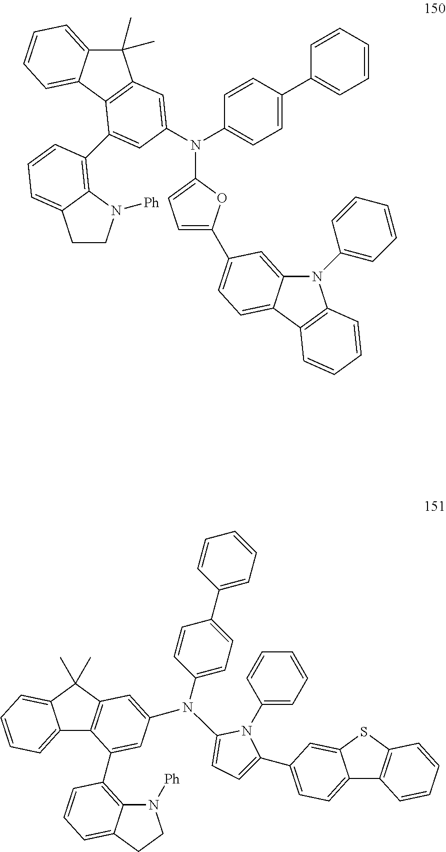 [SMILES: CC1(C)c2ccccc2-c2c(-c3cccc4c3N(c3ccccc3)CC4)cc(N(c3ccc(-c4ccccc4)cc3)c3ccc(-c4ccc5c6ccccc6n(-c6ccccc6)c5c4)o3)cc21 ; CC1(C)c2ccccc2-c2c(-c3cccc4c3N(c3ccccc3)CC4)cc(N(c3ccc(-c4ccccc4)cc3)c3ccc(-c4ccc5c(c4)sc4ccccc45)n3-c3ccccc3)cc21]

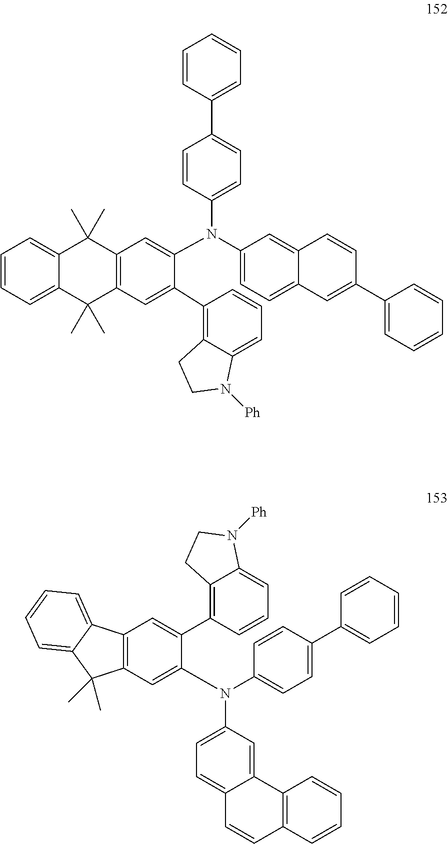 [SMILES: CC1(C)c2ccccc2C(C)(C)c2cc(N(c3ccc(-c4ccccc4)cc3)c3ccc4cc(-c5ccccc5)ccc4c3)c(-c3cccc4c3CCN4c3ccccc3)cc21 ; CC1(C)c2ccccc2-c2cc(-c3cccc4c3CCN4c3ccccc3)c(N(c3ccc(-c4ccccc4)cc3)c3ccc4ccc5ccccc5c4c3)cc21]

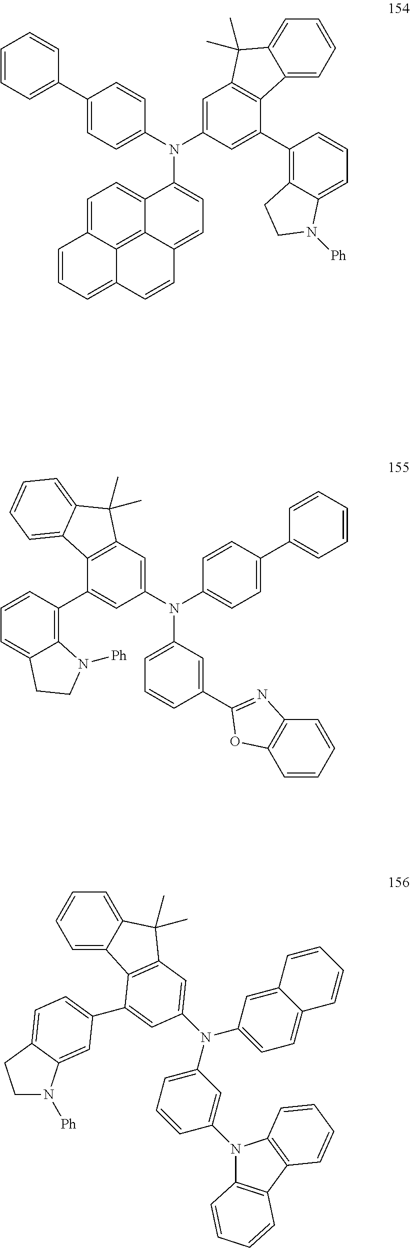 [SMILES: CC1(C)c2ccccc2-c2c(-c3cccc4c3CCN4c3ccccc3)cc(N(c3ccc(-c4ccccc4)cc3)c3ccc4ccc5cccc6ccc3c4c56)cc21 ; CC1(C)c2ccccc2-c2c(-c3cccc4c3N(c3ccccc3)CC4)cc(N(c3ccc(-c4ccccc4)cc3)c3cccc(-c4nc5ccccc5o4)c3)cc21 ; CC1(C)c2ccccc2-c2c(-c3ccc4c(c3)N(c3ccccc3)CC4)cc(N(c3cccc(-n4c5ccccc5c5ccccc54)c3)c3ccc4ccccc4c3)cc21]

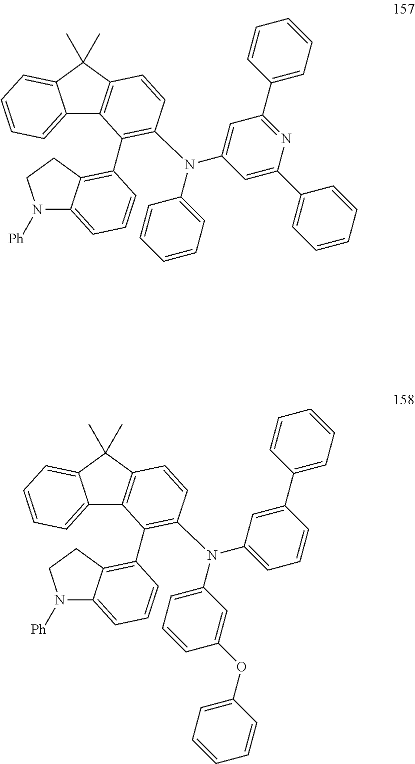 [SMILES: CC1(C)c2ccccc2-c2c1ccc(N(c1ccccc1)c1cc(-c3ccccc3)nc(-c3ccccc3)c1)c2-c1cccc2c1CCN2c1ccccc1 ; CC1(C)c2ccccc2-c2c1ccc(N(c1cccc(Oc3ccccc3)c1)c1cccc(-c3ccccc3)c1)c2-c1cccc2c1CCN2c1ccccc1]

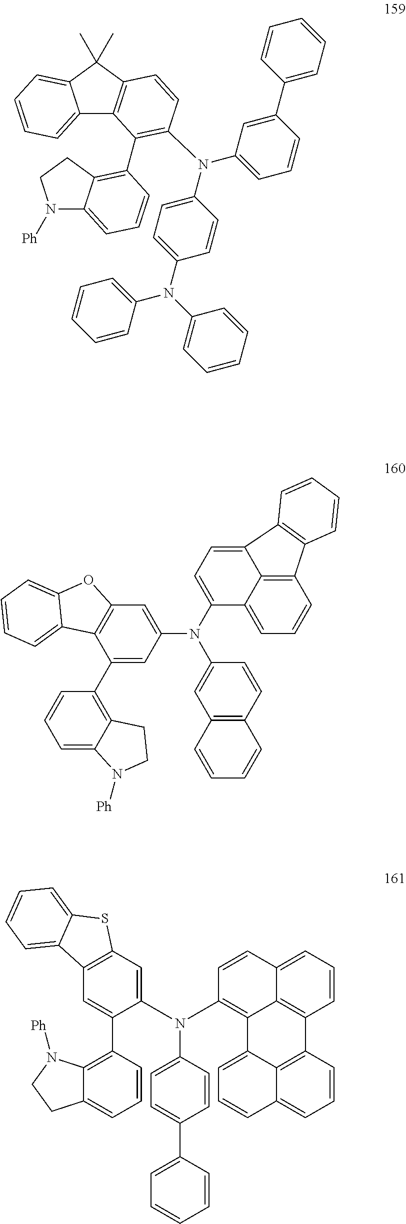 [SMILES: CC1(C)c2ccccc2-c2c1ccc(N(c1ccc(N(c3ccccc3)c3ccccc3)cc1)c1cccc(-c3ccccc3)c1)c2-c1cccc2c1CCN2c1ccccc1 ; c1ccc(N2CCc3c(-c4cc(N(c5ccc6ccccc6c5)c5ccc6c7c(cccc57)-c5ccccc5-6)cc5oc6ccccc6c45)cccc32)cc1 ; c1ccc(-c2ccc(N(c3cc4sc5ccccc5c4cc3-c3cccc4c3N(c3ccccc3)CC4)c3ccc4cccc5c6cccc7cccc(c3c45)c76)cc2)cc1]

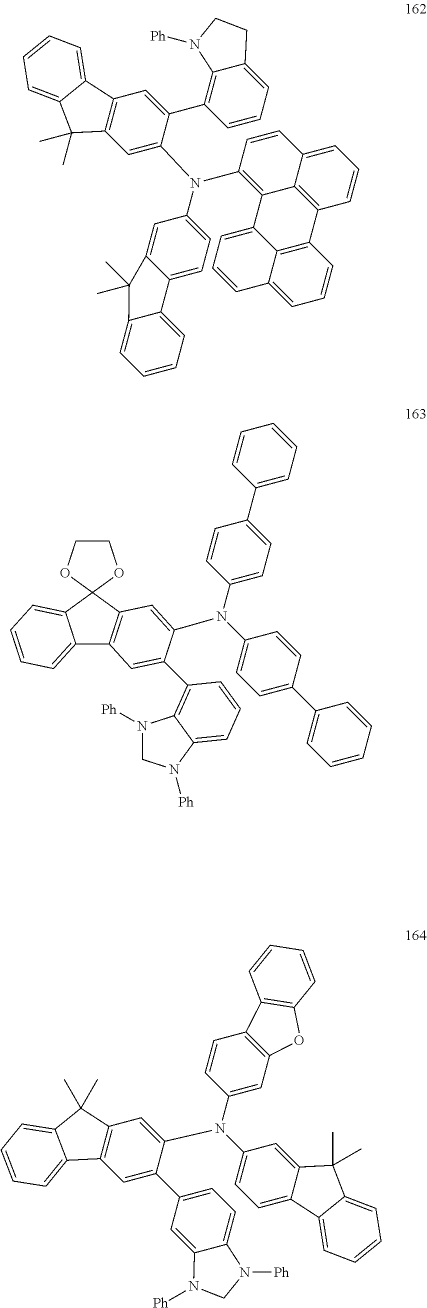 [SMILES: CC1(C)c2ccccc2-c2ccc(N(c3cc4c(cc3-c3cccc5c3N(c3ccccc3)CC5)-c3ccccc3C4(C)C)c3ccc4cccc5c6cccc7cccc(c3c45)c76)cc21 ; c1ccc(-c2ccc(N(c3ccc(-c4ccccc4)cc3)c3cc4c(cc3-c3cccc5c3N(c3ccccc3)CN5c3ccccc3)-c3ccccc3C43OCCO3)cc2)cc1 ; CC1(C)c2ccccc2-c2ccc(N(c3ccc4c(c3)oc3ccccc34)c3cc4c(cc3-c3ccc5c(c3)N(c3ccccc3)CN5c3ccccc3)-c3ccccc3C4(C)C)cc21]

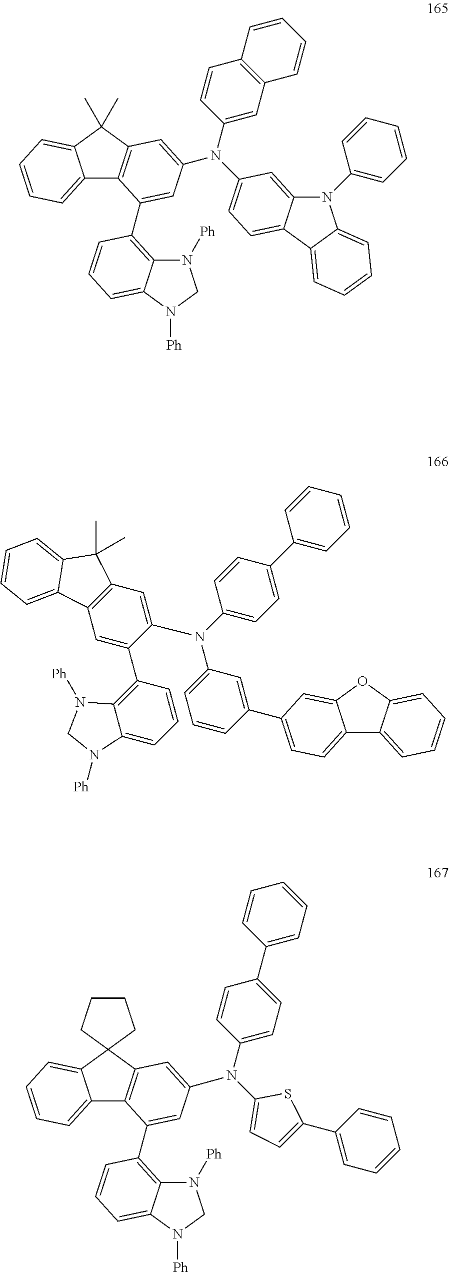 [SMILES: CC1(C)c2ccccc2-c2c(-c3cccc4c3N(c3ccccc3)CN4c3ccccc3)cc(N(c3ccc4ccccc4c3)c3ccc4c5ccccc5n(-c5ccccc5)c4c3)cc21 ; CC1(C)c2ccccc2-c2cc(-c3cccc4c3N(c3ccccc3)CN4c3ccccc3)c(N(c3ccc(-c4ccccc4)cc3)c3cccc(-c4ccc5c(c4)oc4ccccc45)c3)cc21 ; c1ccc(-c2ccc(N(c3cc(-c4cccc5c4N(c4ccccc4)CN5c4ccccc4)c4c(c3)C3(CCCC3)c3ccccc3-4)c3ccc(-c4ccccc4)s3)cc2)cc1]

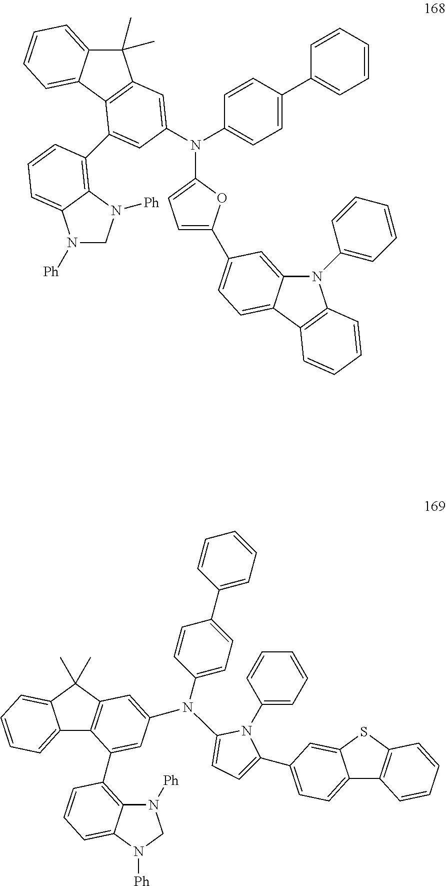 [SMILES: CC1(C)c2ccccc2-c2c(-c3cccc4c3N(c3ccccc3)CN4c3ccccc3)cc(N(c3ccc(-c4ccccc4)cc3)c3ccc(-c4ccc5c6ccccc6n(-c6ccccc6)c5c4)o3)cc21 ; CC1(C)c2ccccc2-c2c(-c3cccc4c3N(c3ccccc3)CN4c3ccccc3)cc(N(c3ccc(-c4ccccc4)cc3)c3ccc(-c4ccc5c(c4)sc4ccccc45)n3-c3ccccc3)cc21]

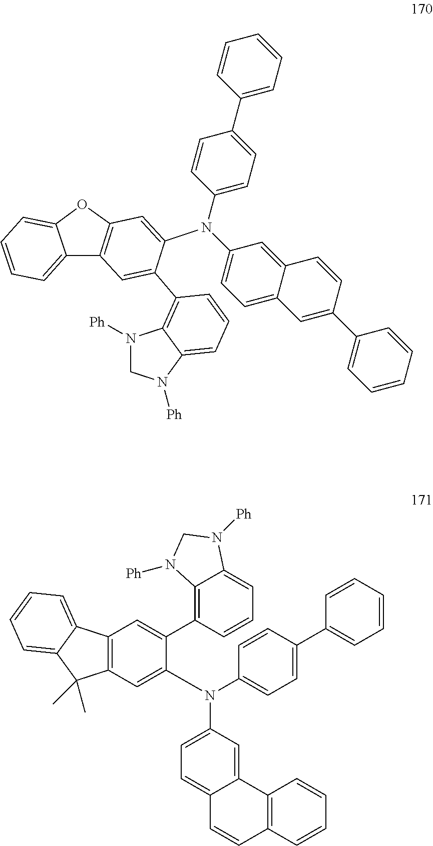 [SMILES: c1ccc(-c2ccc(N(c3ccc4cc(-c5ccccc5)ccc4c3)c3cc4oc5ccccc5c4cc3-c3cccc4c3N(c3ccccc3)CN4c3ccccc3)cc2)cc1 ; CC1(C)c2ccccc2-c2cc(-c3cccc4c3N(c3ccccc3)CN4c3ccccc3)c(N(c3ccc(-c4ccccc4)cc3)c3ccc4ccc5ccccc5c4c3)cc21]

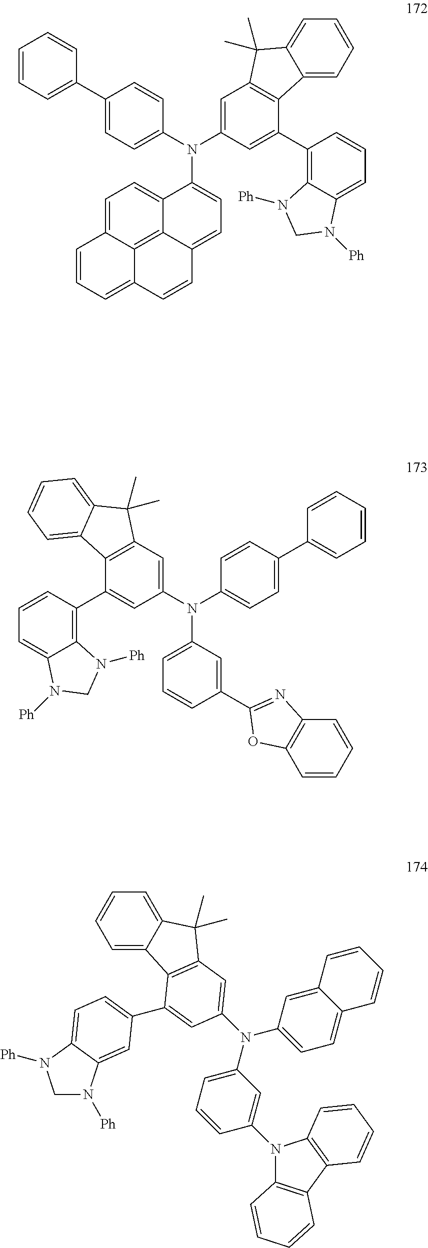 [SMILES: CC1(C)c2ccccc2-c2c(-c3cccc4c3N(c3ccccc3)CN4c3ccccc3)cc(N(c3ccc(-c4ccccc4)cc3)c3ccc4ccc5cccc6ccc3c4c56)cc21 ; CC1(C)c2ccccc2-c2c(-c3cccc4c3N(c3ccccc3)CN4c3ccccc3)cc(N(c3ccc(-c4ccccc4)cc3)c3cccc(-c4nc5ccccc5o4)c3)cc21 ; CC1(C)c2ccccc2-c2c(-c3ccc4c(c3)N(c3ccccc3)CN4c3ccccc3)cc(N(c3cccc(-n4c5ccccc5c5ccccc54)c3)c3ccc4ccccc4c3)cc21]

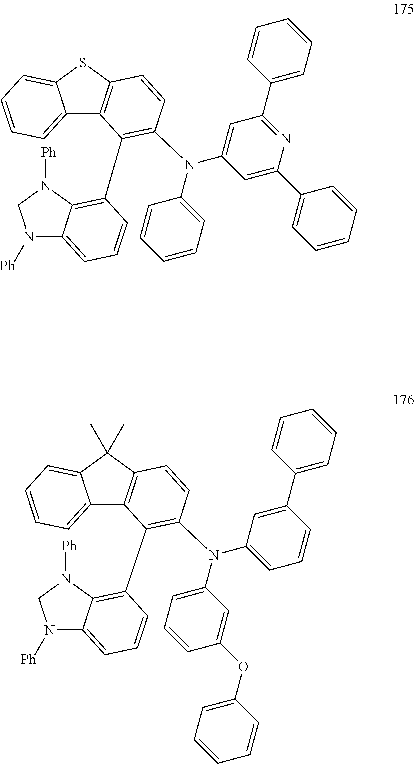 [SMILES: c1ccc(-c2cc(N(c3ccccc3)c3ccc4sc5ccccc5c4c3-c3cccc4c3N(c3ccccc3)CN4c3ccccc3)cc(-c3ccccc3)n2)cc1 ; CC1(C)c2ccccc2-c2c1ccc(N(c1cccc(Oc3ccccc3)c1)c1cccc(-c3ccccc3)c1)c2-c1cccc2c1N(c1ccccc1)CN2c1ccccc1]

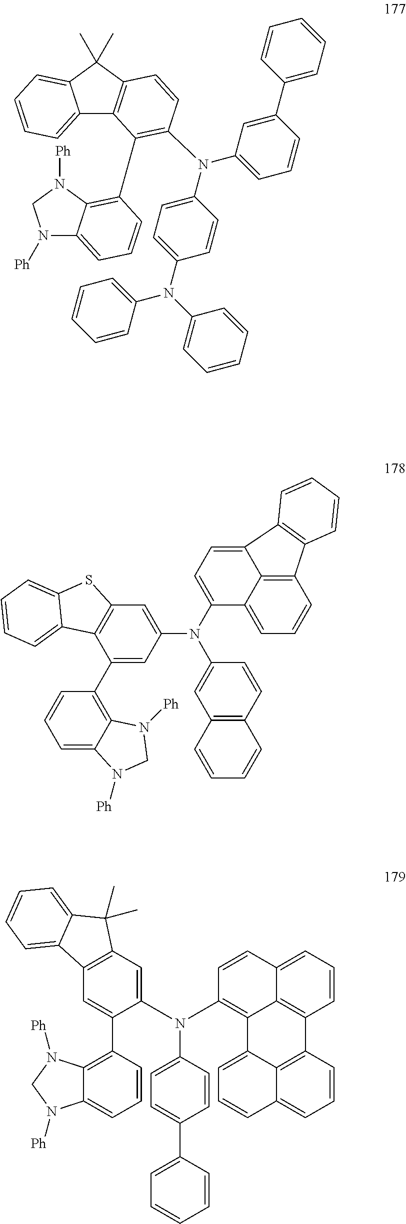 [SMILES: CC1(C)c2ccccc2-c2c1ccc(N(c1ccc(N(c3ccccc3)c3ccccc3)cc1)c1cccc(-c3ccccc3)c1)c2-c1cccc2c1N(c1ccccc1)CN2c1ccccc1 ; c1ccc(N2CN(c3ccccc3)c3c(-c4cc(N(c5ccc6ccccc6c5)c5ccc6c7c(cccc57)-c5ccccc5-6)cc5sc6ccccc6c45)cccc32)cc1 ; CC1(C)c2ccccc2-c2cc(-c3cccc4c3N(c3ccccc3)CN4c3ccccc3)c(N(c3ccc(-c4ccccc4)cc3)c3ccc4cccc5c6cccc7cccc(c3c45)c76)cc21]

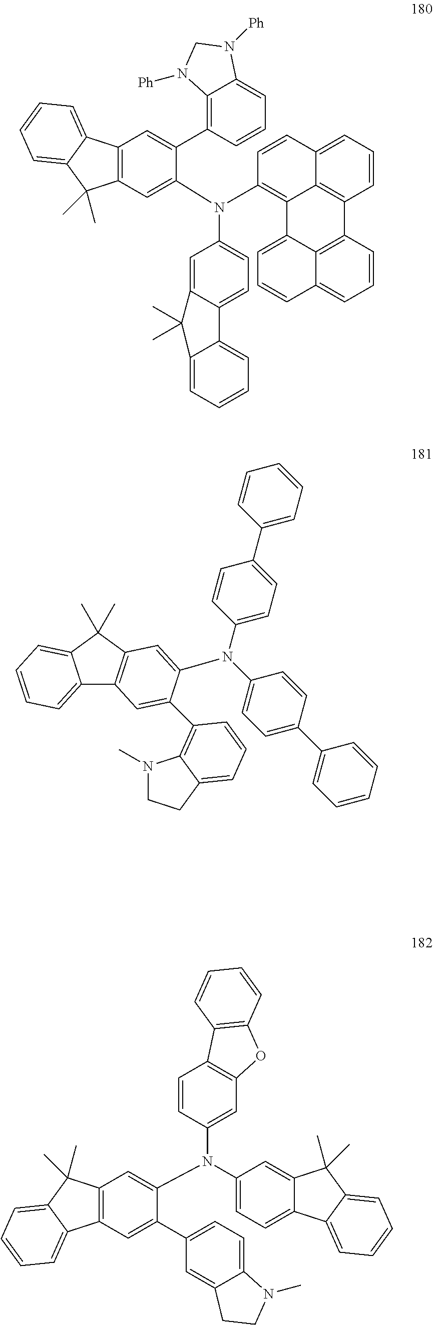 [SMILES: CC1(C)c2ccccc2-c2ccc(N(c3cc4c(cc3-c3cccc5c3N(c3ccccc3)CN5c3ccccc3)-c3ccccc3C4(C)C)c3ccc4cccc5c6cccc7cccc(c3c45)c76)cc21 ; CN1CCc2cccc(-c3cc4c(cc3N(c3ccc(-c5ccccc5)cc3)c3ccc(-c5ccccc5)cc3)C(C)(C)c3ccccc3-4)c21 ; CN1CCc2cc(-c3cc4c(cc3N(c3ccc5c(c3)C(C)(C)c3ccccc3-5)c3ccc5c(c3)oc3ccccc35)C(C)(C)c3ccccc3-4)ccc21]

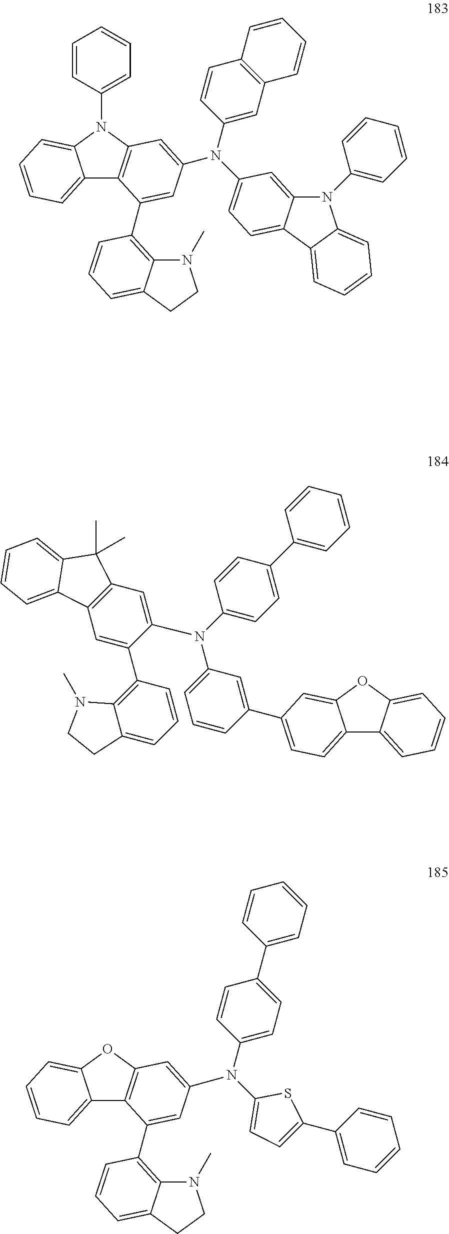 [SMILES: CN1CCc2cccc(-c3cc(N(c4ccc5ccccc5c4)c4ccc5c6ccccc6n(-c6ccccc6)c5c4)cc4c3c3ccccc3n4-c3ccccc3)c21 ; CN1CCc2cccc(-c3cc4c(cc3N(c3ccc(-c5ccccc5)cc3)c3cccc(-c5ccc6c(c5)oc5ccccc56)c3)C(C)(C)c3ccccc3-4)c21 ; CN1CCc2cccc(-c3cc(N(c4ccc(-c5ccccc5)cc4)c4ccc(-c5ccccc5)s4)cc4oc5ccccc5c34)c21]

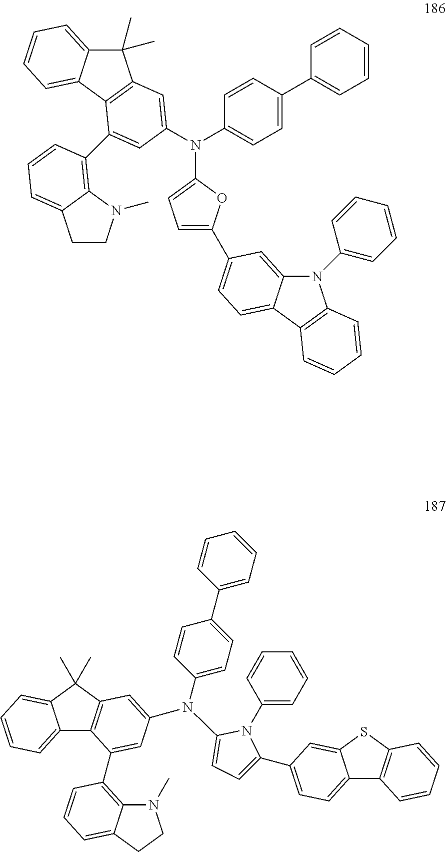 [SMILES: CN1CCc2cccc(-c3cc(N(c4ccc(-c5ccccc5)cc4)c4ccc(-c5ccc6c7ccccc7n(-c7ccccc7)c6c5)o4)cc4c3-c3ccccc3C4(C)C)c21 ; CN1CCc2cccc(-c3cc(N(c4ccc(-c5ccccc5)cc4)c4ccc(-c5ccc6c(c5)sc5ccccc56)n4-c4ccccc4)cc4c3-c3ccccc3C4(C)C)c21]

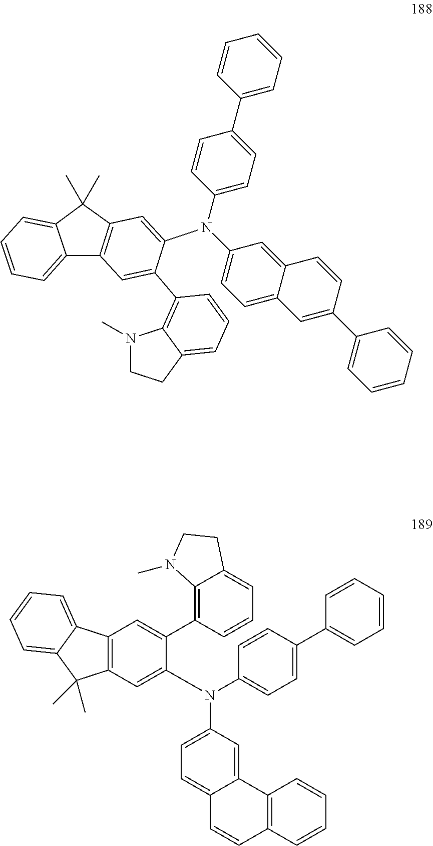 [SMILES: CN1CCc2cccc(-c3cc4c(cc3N(c3ccc(-c5ccccc5)cc3)c3ccc5cc(-c6ccccc6)ccc5c3)C(C)(C)c3ccccc3-4)c21 ; CN1CCc2cccc(-c3cc4c(cc3N(c3ccc(-c5ccccc5)cc3)c3ccc5ccc6ccccc6c5c3)C(C)(C)c3ccccc3-4)c21]

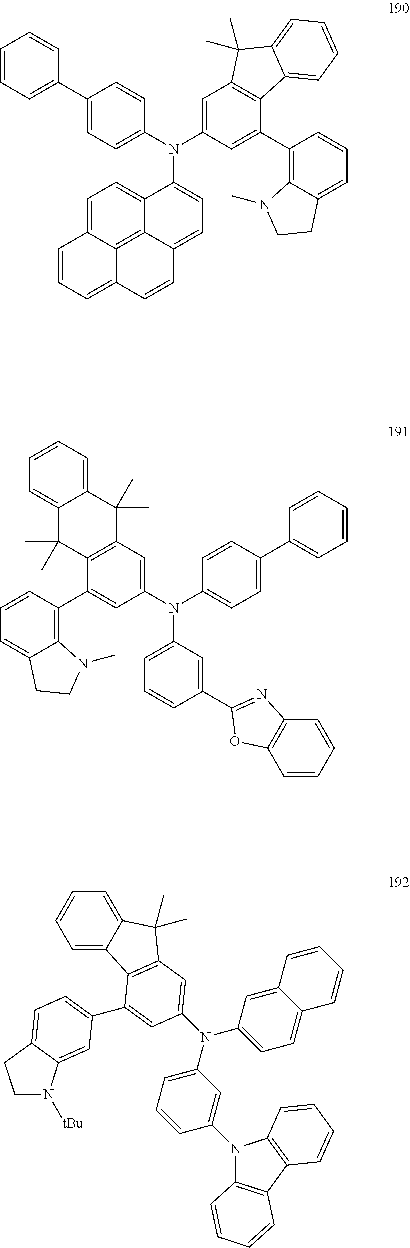 [SMILES: CN1CCc2cccc(-c3cc(N(c4ccc(-c5ccccc5)cc4)c4ccc5ccc6cccc7ccc4c5c67)cc4c3-c3ccccc3C4(C)C)c21 ; CN1CCc2cccc(-c3cc(N(c4ccc(-c5ccccc5)cc4)c4cccc(-c5nc6ccccc6o5)c4)cc4c3C(C)(C)c3ccccc3C4(C)C)c21 ; CC1(C)c2ccccc2-c2c(-c3ccc4c(c3)N(C(C)(C)C)CC4)cc(N(c3cccc(-n4c5ccccc5c5ccccc54)c3)c3ccc4ccccc4c3)cc21]

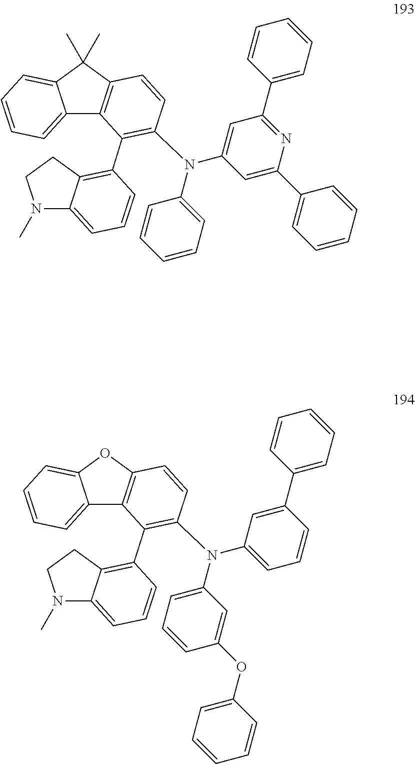 [SMILES: CN1CCc2c(-c3c(N(c4ccccc4)c4cc(-c5ccccc5)nc(-c5ccccc5)c4)ccc4c3-c3ccccc3C4(C)C)cccc21 ; CN1CCc2c(-c3c(N(c4cccc(Oc5ccccc5)c4)c4cccc(-c5ccccc5)c4)ccc4oc5ccccc5c34)cccc21]

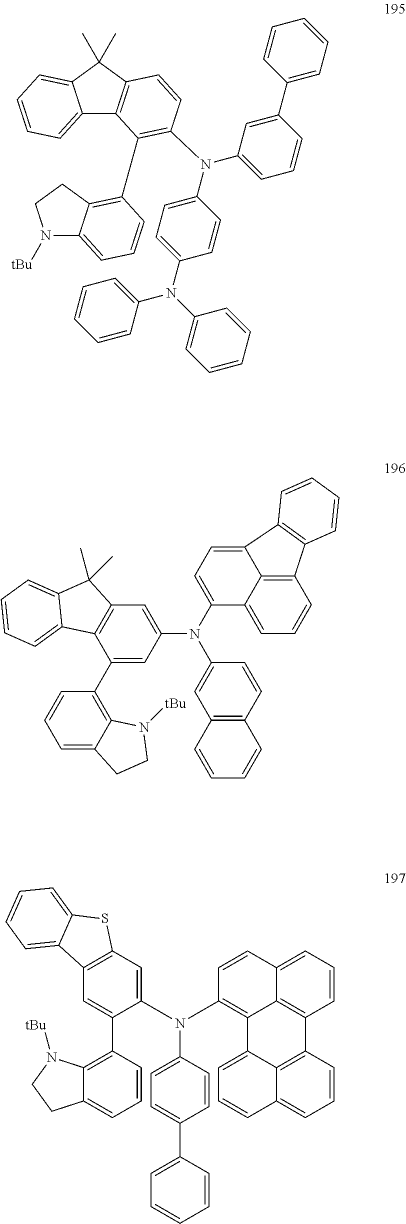 [SMILES: CC1(C)c2ccccc2-c2c1ccc(N(c1ccc(N(c3ccccc3)c3ccccc3)cc1)c1cccc(-c3ccccc3)c1)c2-c1cccc2c1CCN2C(C)(C)C ; CC1(C)c2ccccc2-c2c(-c3cccc4c3N(C(C)(C)C)CC4)cc(N(c3ccc4ccccc4c3)c3ccc4c5c(cccc35)-c3ccccc3-4)cc21 ; CC(C)(C)N1CCc2cccc(-c3cc4c(cc3N(c3ccc(-c5ccccc5)cc3)c3ccc5cccc6c7cccc8cccc(c3c56)c87)sc3ccccc34)c21]

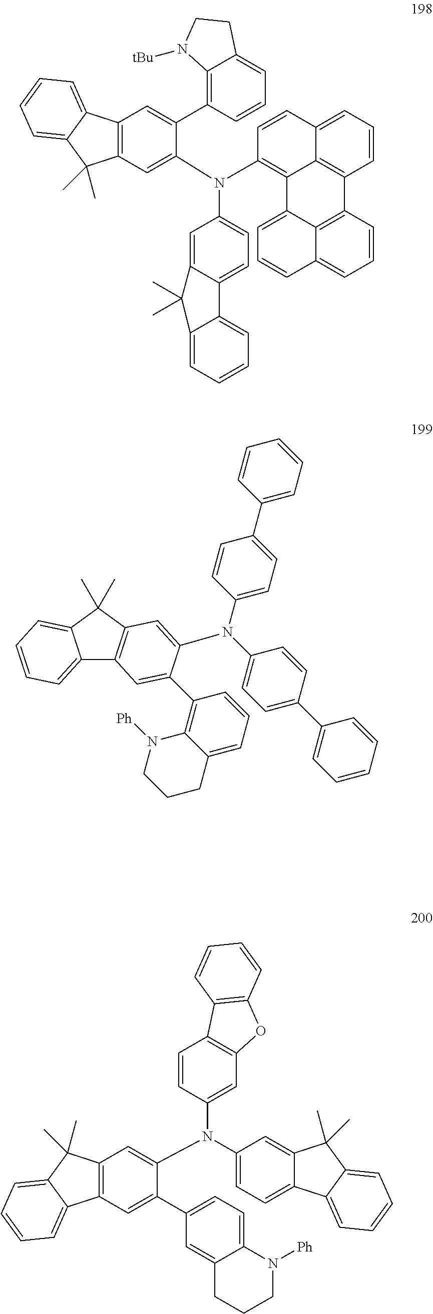 [SMILES: CC1(C)c2ccccc2-c2ccc(N(c3cc4c(cc3-c3cccc5c3N(C(C)(C)C)CC5)-c3ccccc3C4(C)C)c3ccc4cccc5c6cccc7cccc(c3c45)c76)cc21 ; CC1(C)c2ccccc2-c2cc(-c3cccc4c3N(c3ccccc3)CCC4)c(N(c3ccc(-c4ccccc4)cc3)c3ccc(-c4ccccc4)cc3)cc21 ; CC1(C)c2ccccc2-c2ccc(N(c3ccc4c(c3)oc3ccccc34)c3cc4c(cc3-c3ccc5c(c3)CCCN5c3ccccc3)-c3ccccc3C4(C)C)cc21]

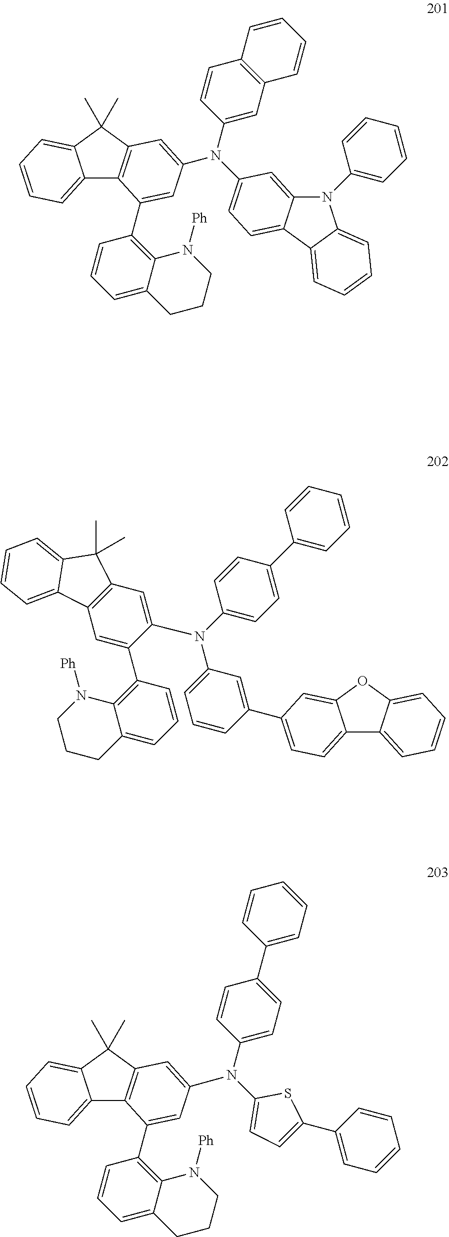 [SMILES: CC1(C)c2ccccc2-c2c(-c3cccc4c3N(c3ccccc3)CCC4)cc(N(c3ccc4ccccc4c3)c3ccc4c5ccccc5n(-c5ccccc5)c4c3)cc21 ; CC1(C)c2ccccc2-c2cc(-c3cccc4c3N(c3ccccc3)CCC4)c(N(c3ccc(-c4ccccc4)cc3)c3cccc(-c4ccc5c(c4)oc4ccccc45)c3)cc21 ; CC1(C)c2ccccc2-c2c(-c3cccc4c3N(c3ccccc3)CCC4)cc(N(c3ccc(-c4ccccc4)cc3)c3ccc(-c4ccccc4)s3)cc21]

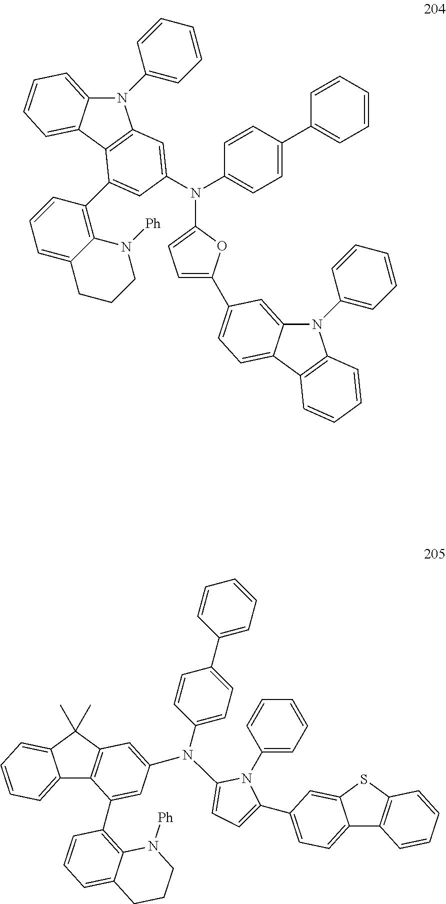 [SMILES: c1ccc(-c2ccc(N(c3cc(-c4cccc5c4N(c4ccccc4)CCC5)c4c5ccccc5n(-c5ccccc5)c4c3)c3ccc(-c4ccc5c6ccccc6n(-c6ccccc6)c5c4)o3)cc2)cc1 ; CC1(C)c2ccccc2-c2c(-c3cccc4c3N(c3ccccc3)CCC4)cc(N(c3ccc(-c4ccccc4)cc3)c3ccc(-c4ccc5c(c4)sc4ccccc45)n3-c3ccccc3)cc21]

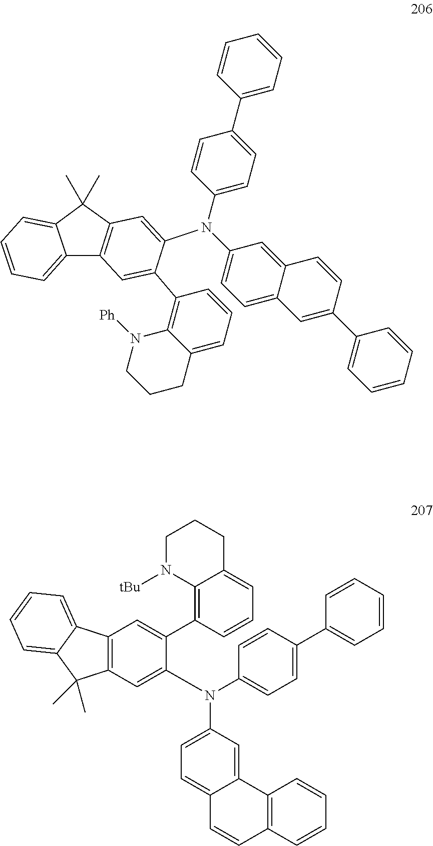 [SMILES: CC1(C)c2ccccc2-c2cc(-c3cccc4c3N(c3ccccc3)CCC4)c(N(c3ccc(-c4ccccc4)cc3)c3ccc4cc(-c5ccccc5)ccc4c3)cc21 ; CC1(C)c2ccccc2-c2cc(-c3cccc4c3N(C(C)(C)C)CCC4)c(N(c3ccc(-c4ccccc4)cc3)c3ccc4ccc5ccccc5c4c3)cc21]

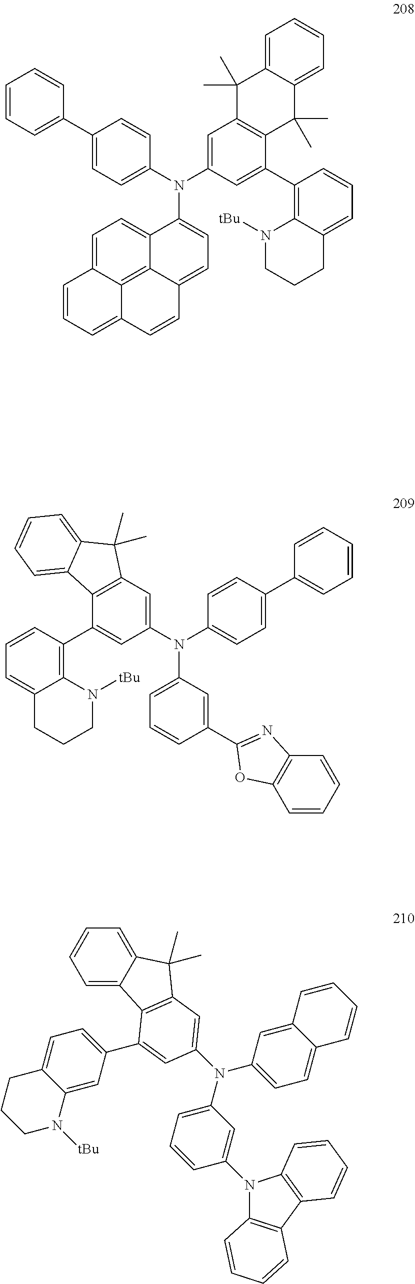 [SMILES: CC1(C)c2ccccc2C(C)(C)c2c(-c3cccc4c3N(C(C)(C)C)CCC4)cc(N(c3ccc(-c4ccccc4)cc3)c3ccc4ccc5cccc6ccc3c4c56)cc21 ; CC1(C)c2ccccc2-c2c(-c3cccc4c3N(C(C)(C)C)CCC4)cc(N(c3ccc(-c4ccccc4)cc3)c3cccc(-c4nc5ccccc5o4)c3)cc21 ; CC1(C)c2ccccc2-c2c(-c3ccc4c(c3)N(C(C)(C)C)CCC4)cc(N(c3cccc(-n4c5ccccc5c5ccccc54)c3)c3ccc4ccccc4c3)cc21]

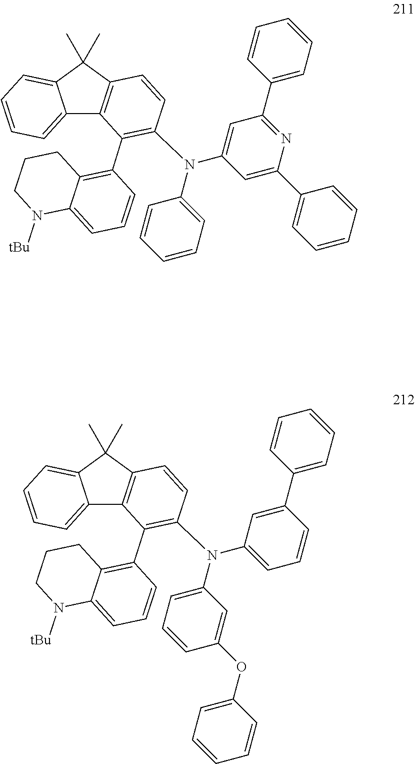 [SMILES: CC1(C)c2ccccc2-c2c1ccc(N(c1ccccc1)c1cc(-c3ccccc3)nc(-c3ccccc3)c1)c2-c1cccc2c1CCCN2C(C)(C)C ; CC1(C)c2ccccc2-c2c1ccc(N(c1cccc(Oc3ccccc3)c1)c1cccc(-c3ccccc3)c1)c2-c1cccc2c1CCCN2C(C)(C)C]

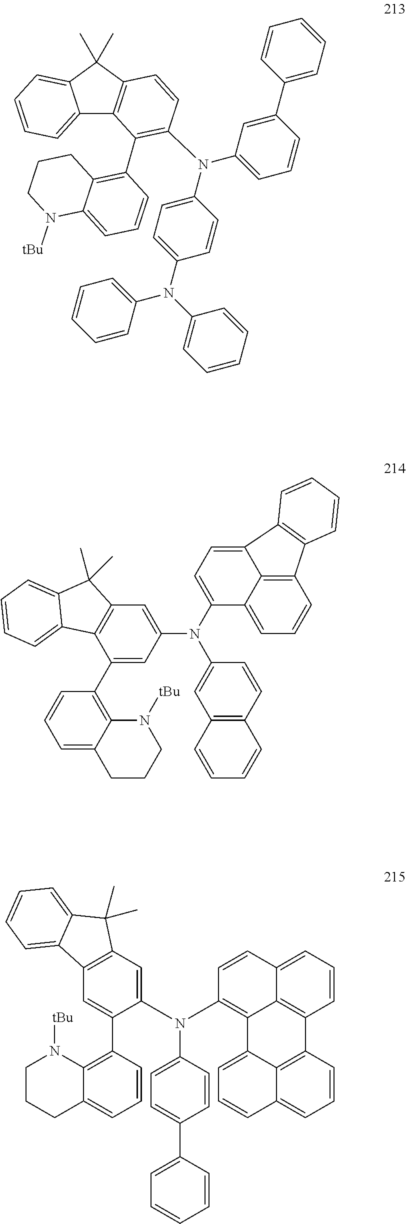 [SMILES: CC1(C)c2ccccc2-c2c1ccc(N(c1ccc(N(c3ccccc3)c3ccccc3)cc1)c1cccc(-c3ccccc3)c1)c2-c1cccc2c1CCCN2C(C)(C)C ; CC1(C)c2ccccc2-c2c(-c3cccc4c3N(C(C)(C)C)CCC4)cc(N(c3ccc4ccccc4c3)c3ccc4c5c(cccc35)-c3ccccc3-4)cc21 ; CC1(C)c2ccccc2-c2cc(-c3cccc4c3N(C(C)(C)C)CCC4)c(N(c3ccc(-c4ccccc4)cc3)c3ccc4cccc5c6cccc7cccc(c3c45)c76)cc21]

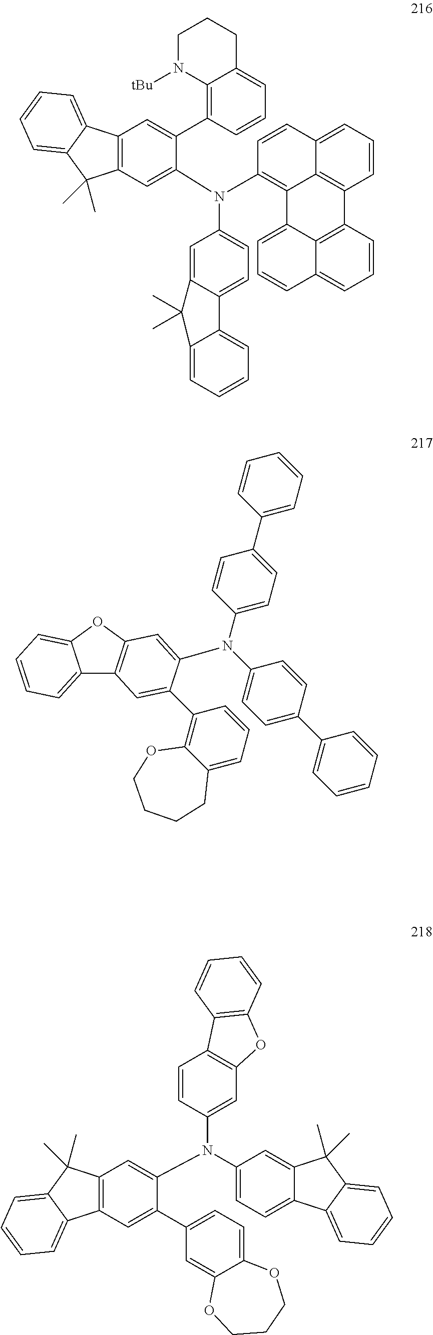 [SMILES: CC1(C)c2ccccc2-c2ccc(N(c3cc4c(cc3-c3cccc5c3N(C(C)(C)C)CCC5)-c3ccccc3C4(C)C)c3ccc4cccc5c6cccc7cccc(c3c45)c76)cc21 ; c1ccc(-c2ccc(N(c3ccc(-c4ccccc4)cc3)c3cc4oc5ccccc5c4cc3-c3cccc4c3OCCCC4)cc2)cc1 ; CC1(C)c2ccccc2-c2ccc(N(c3ccc4c(c3)oc3ccccc34)c3cc4c(cc3-c3ccc5c(c3)OCCCO5)-c3ccccc3C4(C)C)cc21]

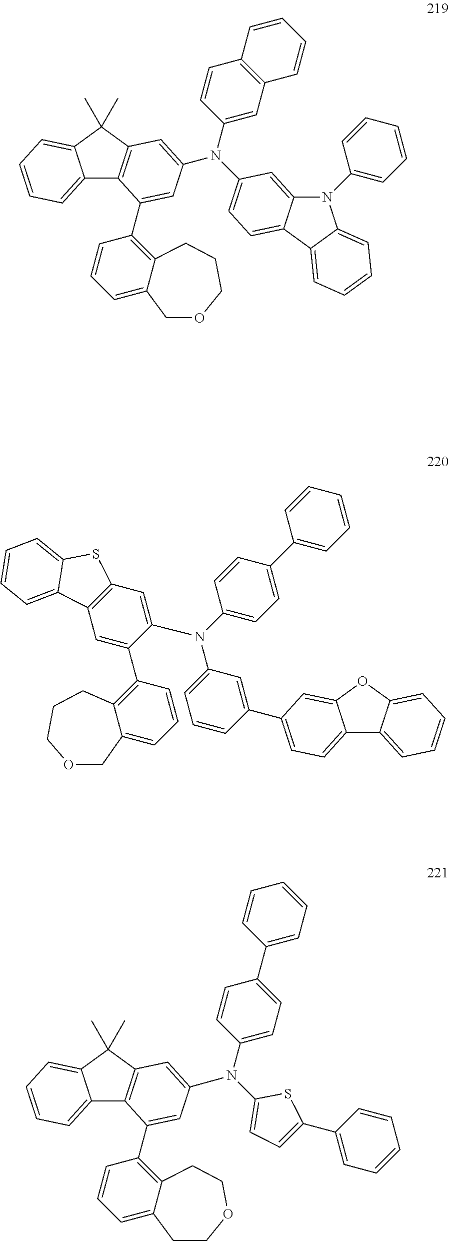 [SMILES: CC1(C)c2ccccc2-c2c(-c3cccc4c3CCCOC4)cc(N(c3ccc4ccccc4c3)c3ccc4c5ccccc5n(-c5ccccc5)c4c3)cc21 ; c1ccc(-c2ccc(N(c3cccc(-c4ccc5c(c4)oc4ccccc45)c3)c3cc4sc5ccccc5c4cc3-c3cccc4c3CCCOC4)cc2)cc1 ; CC1(C)c2ccccc2-c2c(-c3cccc4c3CCOCC4)cc(N(c3ccc(-c4ccccc4)cc3)c3ccc(-c4ccccc4)s3)cc21]

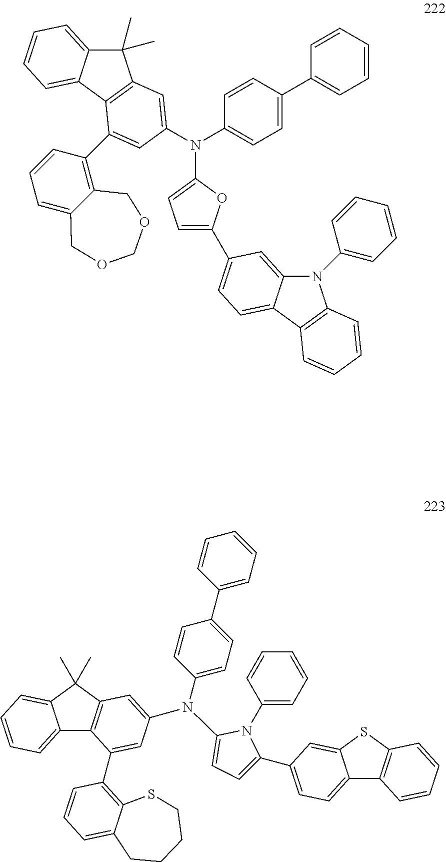 [SMILES: CC1(C)c2ccccc2-c2c(-c3cccc4c3COCOC4)cc(N(c3ccc(-c4ccccc4)cc3)c3ccc(-c4ccc5c6ccccc6n(-c6ccccc6)c5c4)o3)cc21 ; CC1(C)c2ccccc2-c2c(-c3cccc4c3SCCCC4)cc(N(c3ccc(-c4ccccc4)cc3)c3ccc(-c4ccc5c(c4)sc4ccccc45)n3-c3ccccc3)cc21]

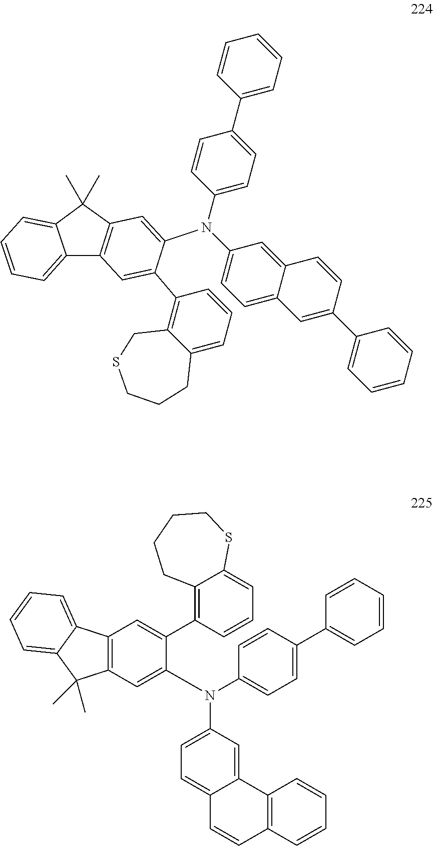 [SMILES: CC1(C)c2ccccc2-c2cc(-c3cccc4c3CSCCC4)c(N(c3ccc(-c4ccccc4)cc3)c3ccc4cc(-c5ccccc5)ccc4c3)cc21 ; CC1(C)c2ccccc2-c2cc(-c3cccc4c3CCCCS4)c(N(c3ccc(-c4ccccc4)cc3)c3ccc4ccc5ccccc5c4c3)cc21]

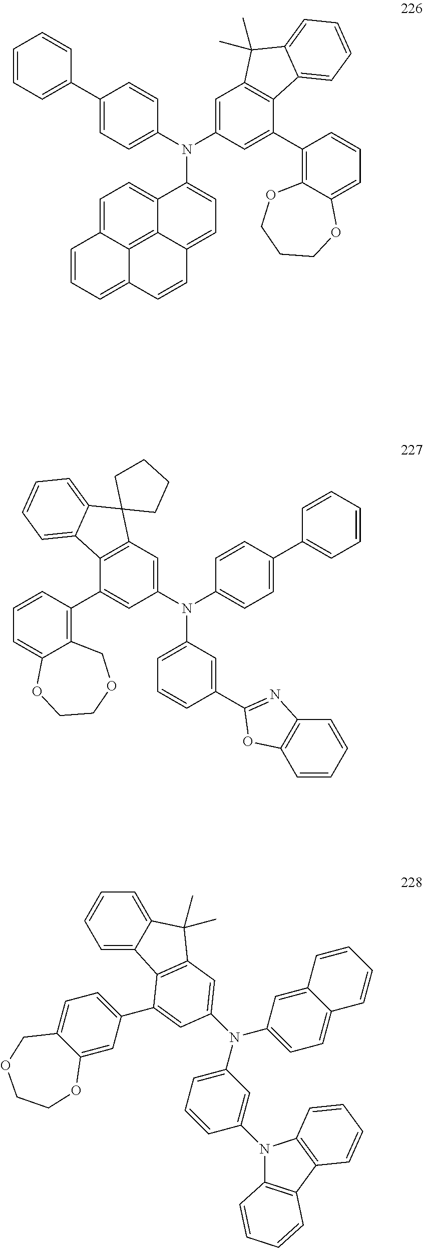 [SMILES: CC1(C)c2ccccc2-c2c(-c3cccc4c3OCCCO4)cc(N(c3ccc(-c4ccccc4)cc3)c3ccc4ccc5cccc6ccc3c4c56)cc21 ; c1ccc(-c2ccc(N(c3cccc(-c4nc5ccccc5o4)c3)c3cc(-c4cccc5c4COCCO5)c4c(c3)C3(CCCC3)c3ccccc3-4)cc2)cc1 ; CC1(C)c2ccccc2-c2c(-c3ccc4c(c3)OCCOC4)cc(N(c3cccc(-n4c5ccccc5c5ccccc54)c3)c3ccc4ccccc4c3)cc21]

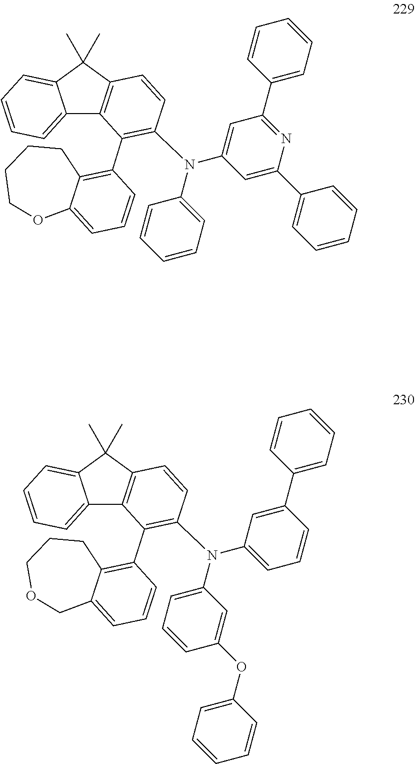 [SMILES: CC1(C)c2ccccc2-c2c1ccc(N(c1ccccc1)c1cc(-c3ccccc3)nc(-c3ccccc3)c1)c2-c1cccc2c1CCCCO2 ; CC1(C)c2ccccc2-c2c1ccc(N(c1cccc(Oc3ccccc3)c1)c1cccc(-c3ccccc3)c1)c2-c1cccc2c1CCCOC2]

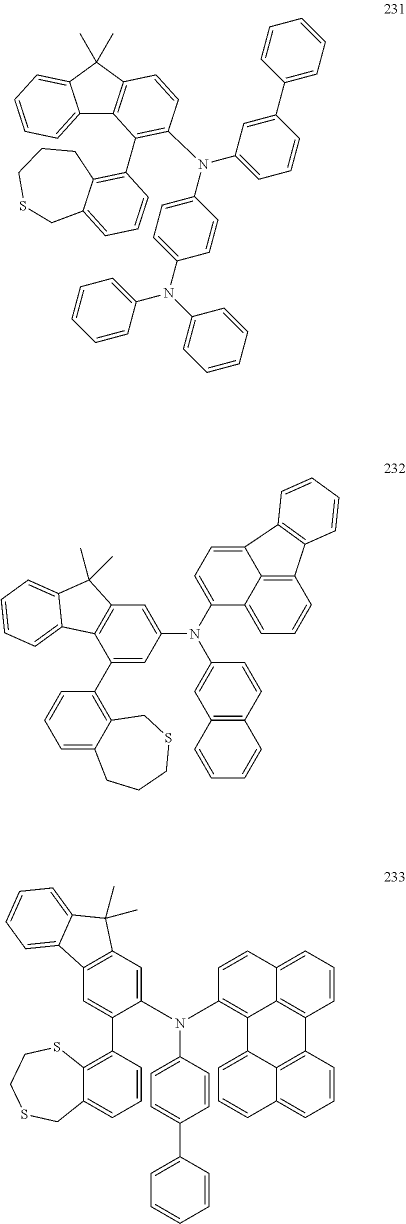 [SMILES: CC1(C)c2ccccc2-c2c1ccc(N(c1ccc(N(c3ccccc3)c3ccccc3)cc1)c1cccc(-c3ccccc3)c1)c2-c1cccc2c1CCCSC2 ; CC1(C)c2ccccc2-c2c(-c3cccc4c3CSCCC4)cc(N(c3ccc4ccccc4c3)c3ccc4c5c(cccc35)-c3ccccc3-4)cc21 ; CC1(C)c2ccccc2-c2cc(-c3cccc4c3SCCSC4)c(N(c3ccc(-c4ccccc4)cc3)c3ccc4cccc5c6cccc7cccc(c3c45)c76)cc21]

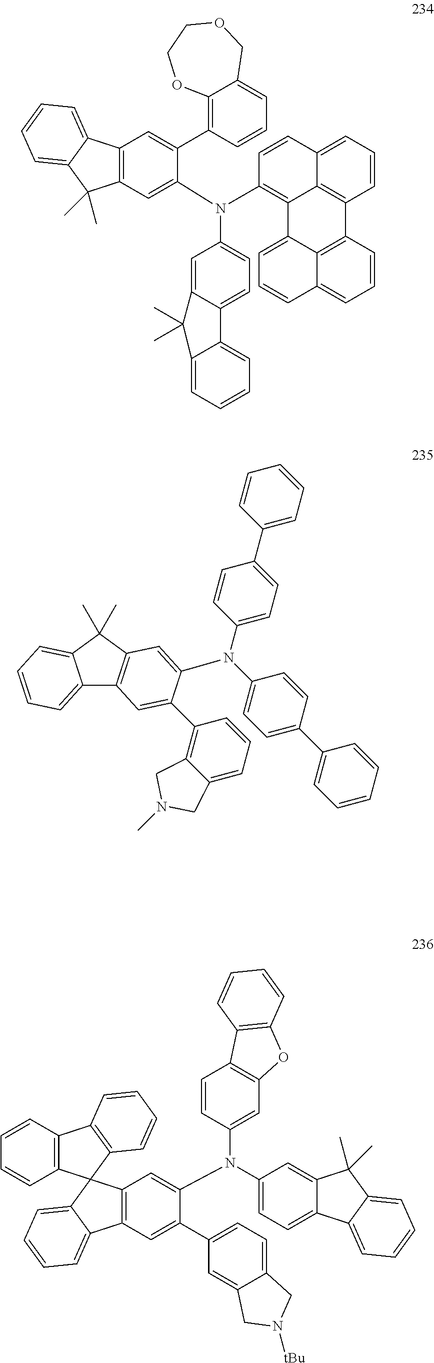 [SMILES: CC1(C)c2ccccc2-c2ccc(N(c3cc4c(cc3-c3cccc5c3OCCOC5)-c3ccccc3C4(C)C)c3ccc4cccc5c6cccc7cccc(c3c45)c76)cc21 ; CN1Cc2cccc(-c3cc4c(cc3N(c3ccc(-c5ccccc5)cc3)c3ccc(-c5ccccc5)cc3)C(C)(C)c3ccccc3-4)c2C1 ; CC1(C)c2ccccc2-c2ccc(N(c3ccc4c(c3)oc3ccccc34)c3cc4c(cc3-c3ccc5c(c3)CN(C(C)(C)C)C5)-c3ccccc3C43c4ccccc4-c4ccccc43)cc21]

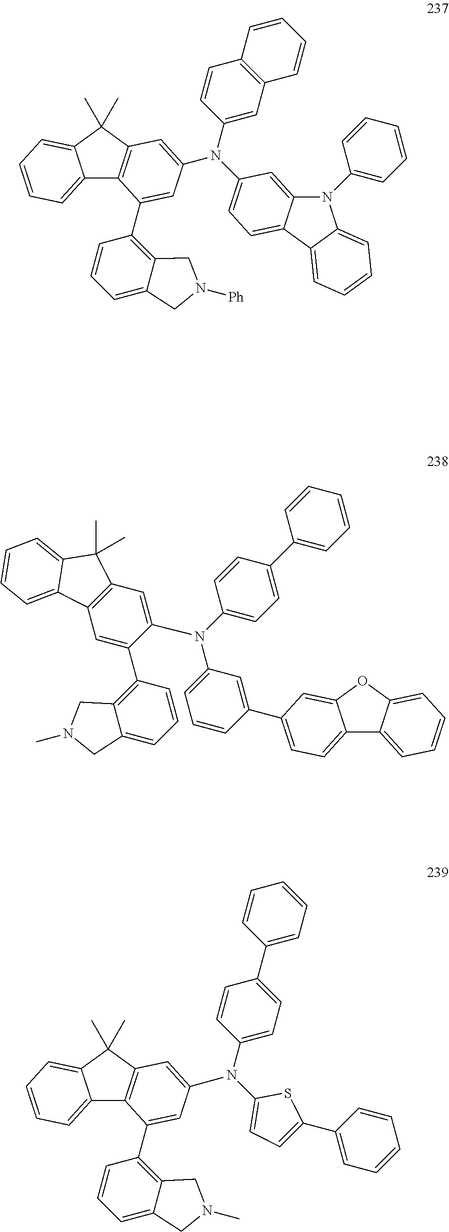 [SMILES: CC1(C)c2ccccc2-c2c(-c3cccc4c3CN(c3ccccc3)C4)cc(N(c3ccc4ccccc4c3)c3ccc4c5ccccc5n(-c5ccccc5)c4c3)cc21 ; CN1Cc2cccc(-c3cc4c(cc3N(c3ccc(-c5ccccc5)cc3)c3cccc(-c5ccc6c(c5)oc5ccccc56)c3)C(C)(C)c3ccccc3-4)c2C1 ; CN1Cc2cccc(-c3cc(N(c4ccc(-c5ccccc5)cc4)c4ccc(-c5ccccc5)s4)cc4c3-c3ccccc3C4(C)C)c2C1]

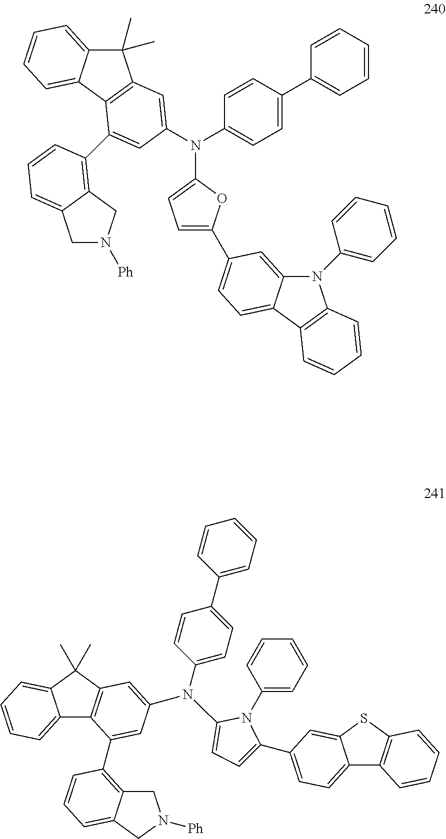 [SMILES: CC1(C)c2ccccc2-c2c(-c3cccc4c3CN(c3ccccc3)C4)cc(N(c3ccc(-c4ccccc4)cc3)c3ccc(-c4ccc5c6ccccc6n(-c6ccccc6)c5c4)o3)cc21 ; CC1(C)c2ccccc2-c2c(-c3cccc4c3CN(c3ccccc3)C4)cc(N(c3ccc(-c4ccccc4)cc3)c3ccc(-c4ccc5c(c4)sc4ccccc45)n3-c3ccccc3)cc21]

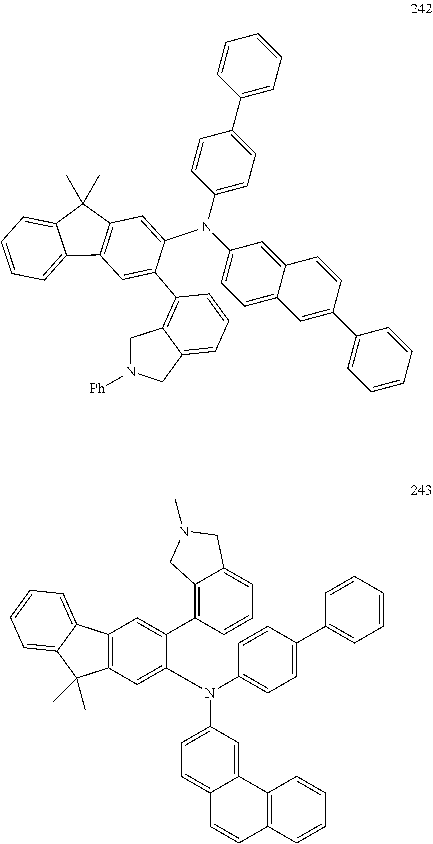 [SMILES: CC1(C)c2ccccc2-c2cc(-c3cccc4c3CN(c3ccccc3)C4)c(N(c3ccc(-c4ccccc4)cc3)c3ccc4cc(-c5ccccc5)ccc4c3)cc21 ; CN1Cc2cccc(-c3cc4c(cc3N(c3ccc(-c5ccccc5)cc3)c3ccc5ccc6ccccc6c5c3)C(C)(C)c3ccccc3-4)c2C1]

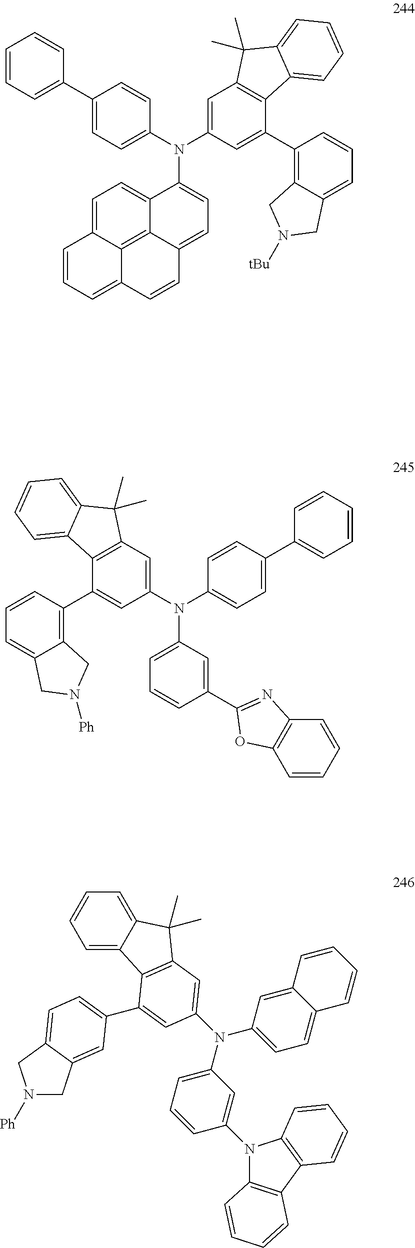 [SMILES: CC1(C)c2ccccc2-c2c(-c3cccc4c3CN(C(C)(C)C)C4)cc(N(c3ccc(-c4ccccc4)cc3)c3ccc4ccc5cccc6ccc3c4c56)cc21 ; CC1(C)c2ccccc2-c2c(-c3cccc4c3CN(c3ccccc3)C4)cc(N(c3ccc(-c4ccccc4)cc3)c3cccc(-c4nc5ccccc5o4)c3)cc21 ; CC1(C)c2ccccc2-c2c(-c3ccc4c(c3)CN(c3ccccc3)C4)cc(N(c3cccc(-n4c5ccccc5c5ccccc54)c3)c3ccc4ccccc4c3)cc21]

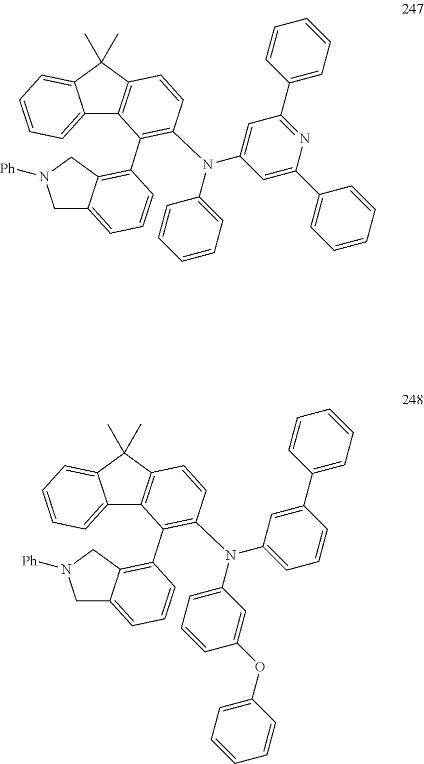 [SMILES: CC1(C)c2ccccc2-c2c1ccc(N(c1ccccc1)c1cc(-c3ccccc3)nc(-c3ccccc3)c1)c2-c1cccc2c1CN(c1ccccc1)C2 ; CC1(C)c2ccccc2-c2c1ccc(N(c1cccc(Oc3ccccc3)c1)c1cccc(-c3ccccc3)c1)c2-c1cccc2c1CN(c1ccccc1)C2]

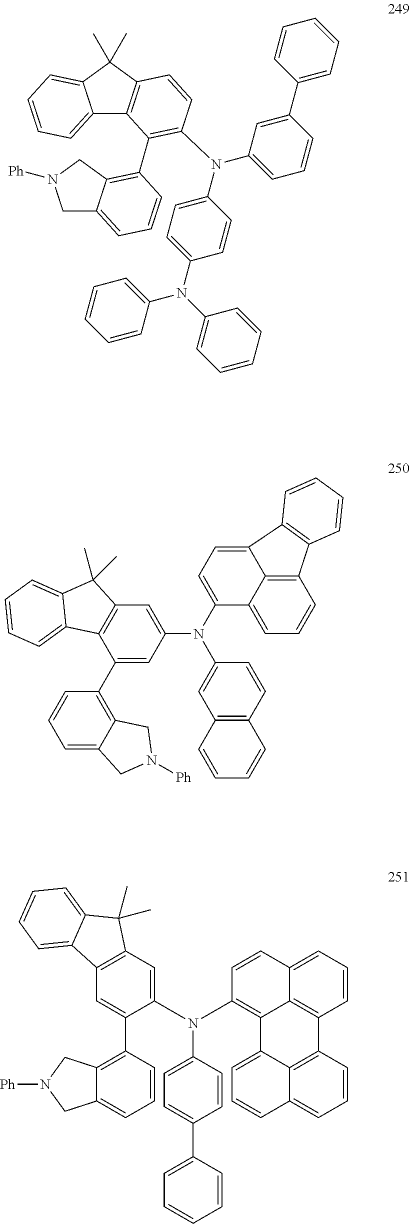 [SMILES: CC1(C)c2ccccc2-c2c1ccc(N(c1ccc(N(c3ccccc3)c3ccccc3)cc1)c1cccc(-c3ccccc3)c1)c2-c1cccc2c1CN(c1ccccc1)C2 ; CC1(C)c2ccccc2-c2c(-c3cccc4c3CN(c3ccccc3)C4)cc(N(c3ccc4ccccc4c3)c3ccc4c5c(cccc35)-c3ccccc3-4)cc21 ; CC1(C)c2ccccc2-c2cc(-c3cccc4c3CN(c3ccccc3)C4)c(N(c3ccc(-c4ccccc4)cc3)c3ccc4cccc5c6cccc7cccc(c3c45)c76)cc21]

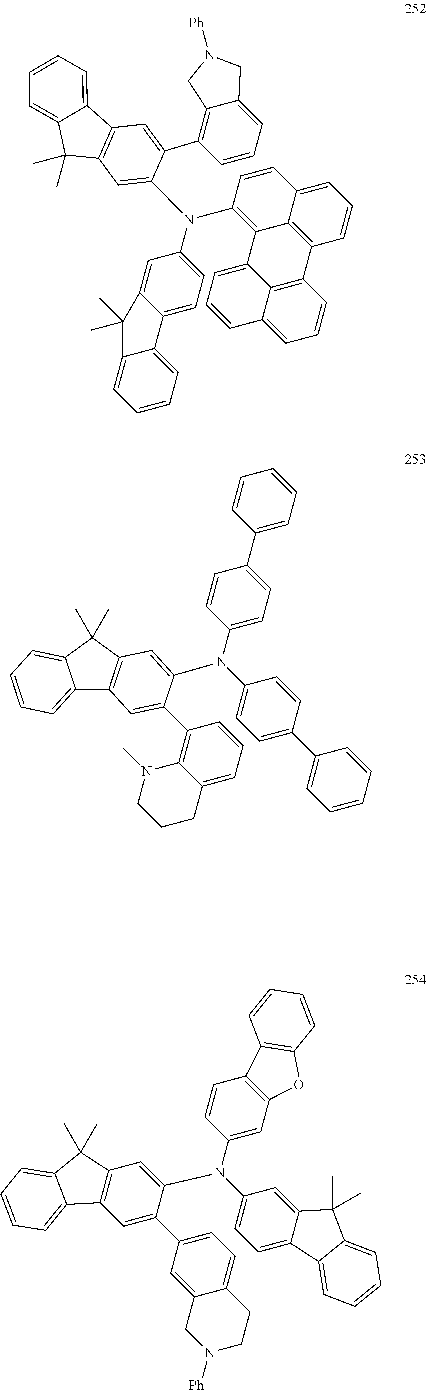 [SMILES: CC1(C)c2ccccc2-c2ccc(N(c3cc4c(cc3-c3cccc5c3CN(c3ccccc3)C5)-c3ccccc3C4(C)C)c3ccc4cccc5c6cccc7cccc(c3c45)c76)cc21 ; CN1CCCc2cccc(-c3cc4c(cc3N(c3ccc(-c5ccccc5)cc3)c3ccc(-c5ccccc5)cc3)C(C)(C)c3ccccc3-4)c21 ; CC1(C)c2ccccc2-c2ccc(N(c3ccc4c(c3)oc3ccccc34)c3cc4c(cc3-c3ccc5c(c3)CN(c3ccccc3)CC5)-c3ccccc3C4(C)C)cc21]

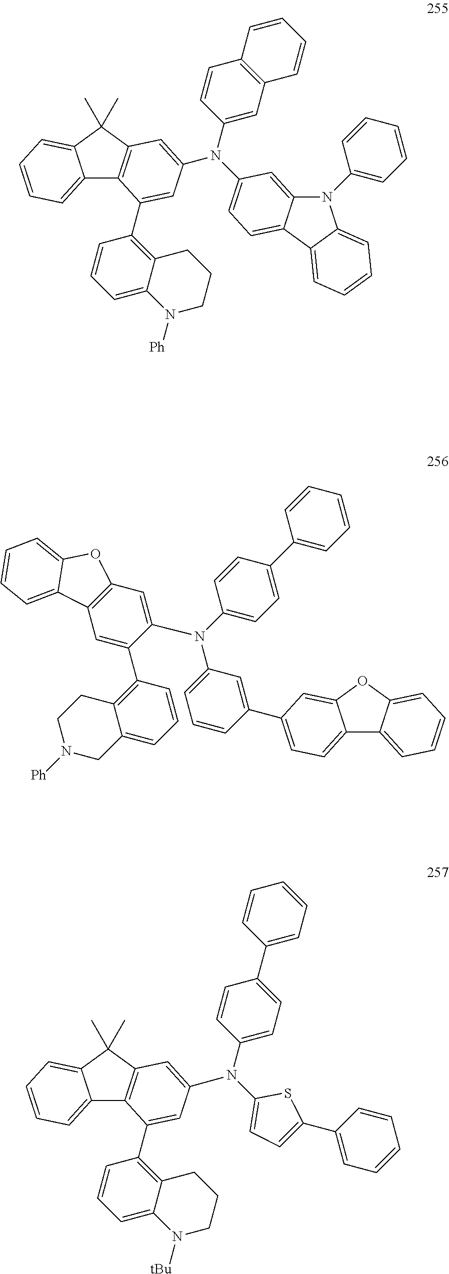 [SMILES: CC1(C)c2ccccc2-c2c(-c3cccc4c3CCCN4c3ccccc3)cc(N(c3ccc4ccccc4c3)c3ccc4c5ccccc5n(-c5ccccc5)c4c3)cc21 ; c1ccc(-c2ccc(N(c3cccc(-c4ccc5c(c4)oc4ccccc45)c3)c3cc4oc5ccccc5c4cc3-c3cccc4c3CCN(c3ccccc3)C4)cc2)cc1 ; CC1(C)c2ccccc2-c2c(-c3cccc4c3CCCN4C(C)(C)C)cc(N(c3ccc(-c4ccccc4)cc3)c3ccc(-c4ccccc4)s3)cc21]

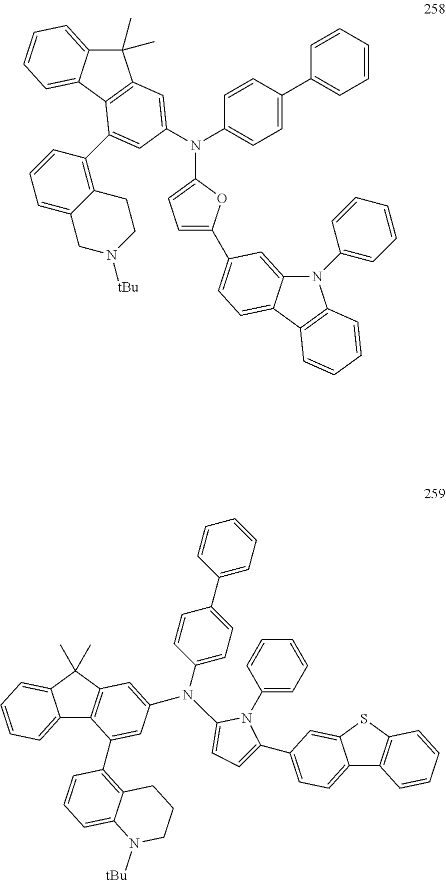 [SMILES: CC1(C)c2ccccc2-c2c(-c3cccc4c3CCN(C(C)(C)C)C4)cc(N(c3ccc(-c4ccccc4)cc3)c3ccc(-c4ccc5c6ccccc6n(-c6ccccc6)c5c4)o3)cc21 ; CC1(C)c2ccccc2-c2c(-c3cccc4c3CCCN4C(C)(C)C)cc(N(c3ccc(-c4ccccc4)cc3)c3ccc(-c4ccc5c(c4)sc4ccccc45)n3-c3ccccc3)cc21]

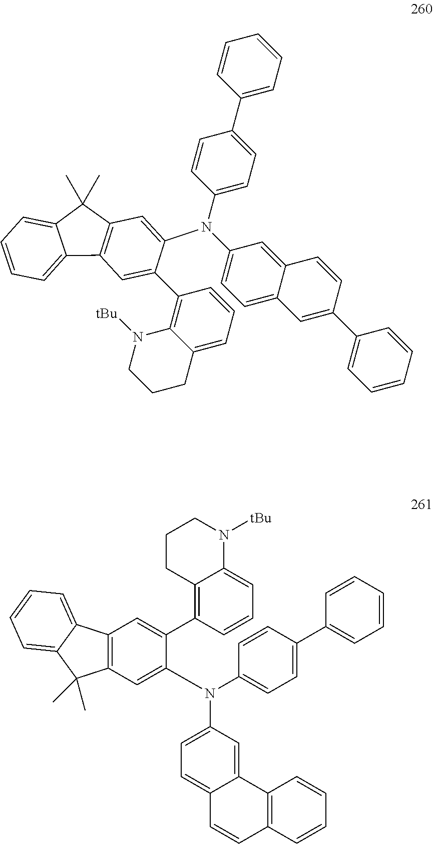 [SMILES: CC1(C)c2ccccc2-c2cc(-c3cccc4c3N(C(C)(C)C)CCC4)c(N(c3ccc(-c4ccccc4)cc3)c3ccc4cc(-c5ccccc5)ccc4c3)cc21 ; CC1(C)c2ccccc2-c2cc(-c3cccc4c3CCCN4C(C)(C)C)c(N(c3ccc(-c4ccccc4)cc3)c3ccc4ccc5ccccc5c4c3)cc21]

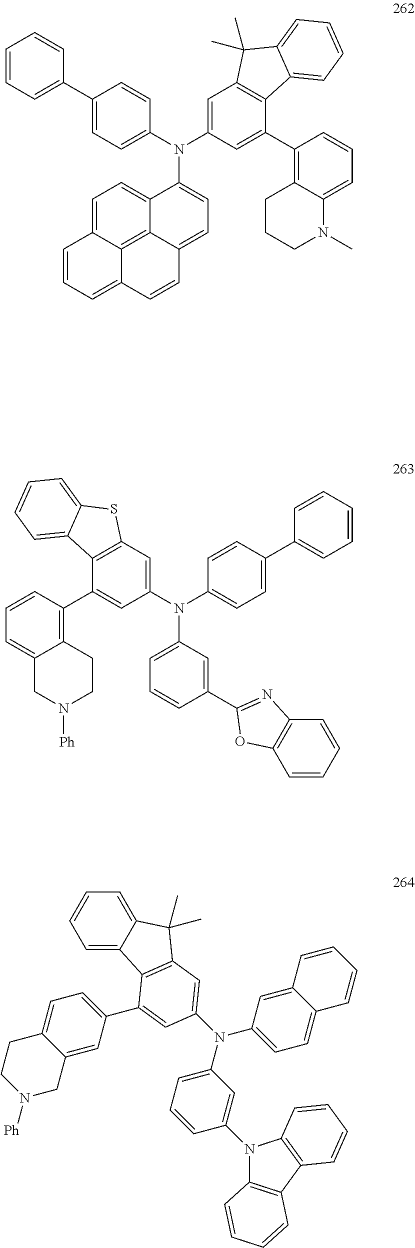 [SMILES: CN1CCCc2c(-c3cc(N(c4ccc(-c5ccccc5)cc4)c4ccc5ccc6cccc7ccc4c5c67)cc4c3-c3ccccc3C4(C)C)cccc21 ; c1ccc(-c2ccc(N(c3cccc(-c4nc5ccccc5o4)c3)c3cc(-c4cccc5c4CCN(c4ccccc4)C5)c4c(c3)sc3ccccc34)cc2)cc1 ; CC1(C)c2ccccc2-c2c(-c3ccc4c(c3)CN(c3ccccc3)CC4)cc(N(c3cccc(-n4c5ccccc5c5ccccc54)c3)c3ccc4ccccc4c3)cc21]

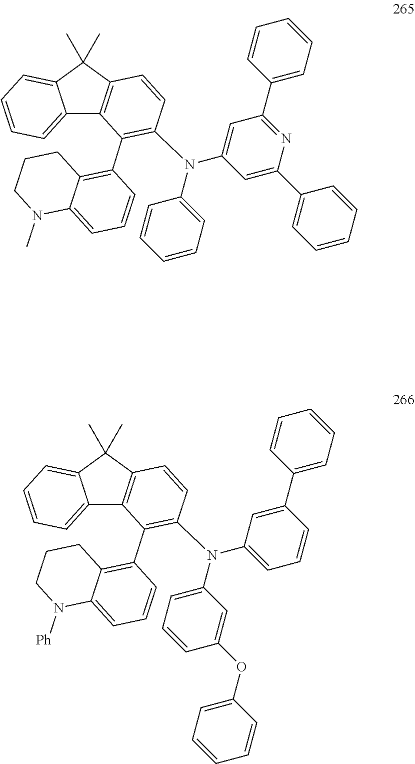 [SMILES: CN1CCCc2c(-c3c(N(c4ccccc4)c4cc(-c5ccccc5)nc(-c5ccccc5)c4)ccc4c3-c3ccccc3C4(C)C)cccc21 ; CC1(C)c2ccccc2-c2c1ccc(N(c1cccc(Oc3ccccc3)c1)c1cccc(-c3ccccc3)c1)c2-c1cccc2c1CCCN2c1ccccc1]

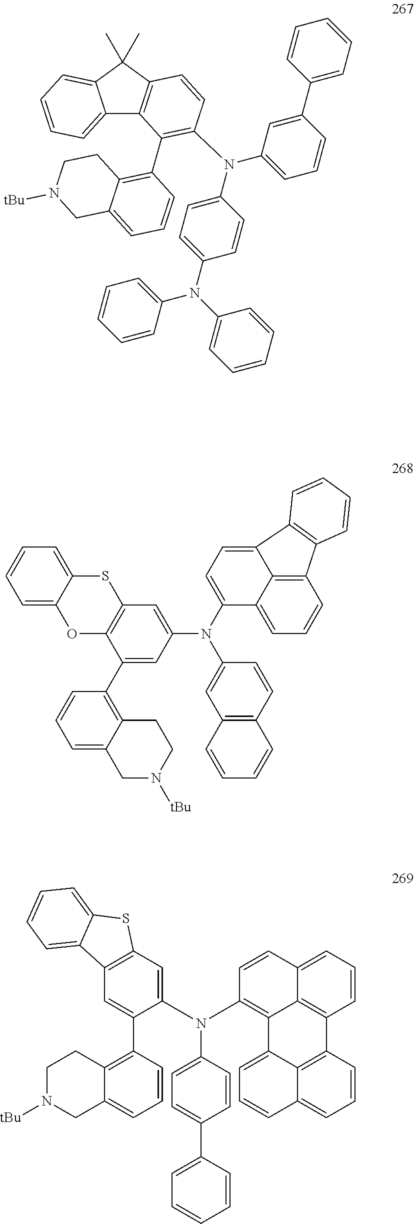 [SMILES: CC1(C)c2ccccc2-c2c1ccc(N(c1ccc(N(c3ccccc3)c3ccccc3)cc1)c1cccc(-c3ccccc3)c1)c2-c1cccc2c1CCN(C(C)(C)C)C2 ; CC(C)(C)N1CCc2c(cccc2-c2cc(N(c3ccc4ccccc4c3)c3ccc4c5c(cccc35)-c3ccccc3-4)cc3c2Oc2ccccc2S3)C1 ; CC(C)(C)N1CCc2c(cccc2-c2cc3c(cc2N(c2ccc(-c4ccccc4)cc2)c2ccc4cccc5c6cccc7cccc(c2c45)c76)sc2ccccc23)C1]

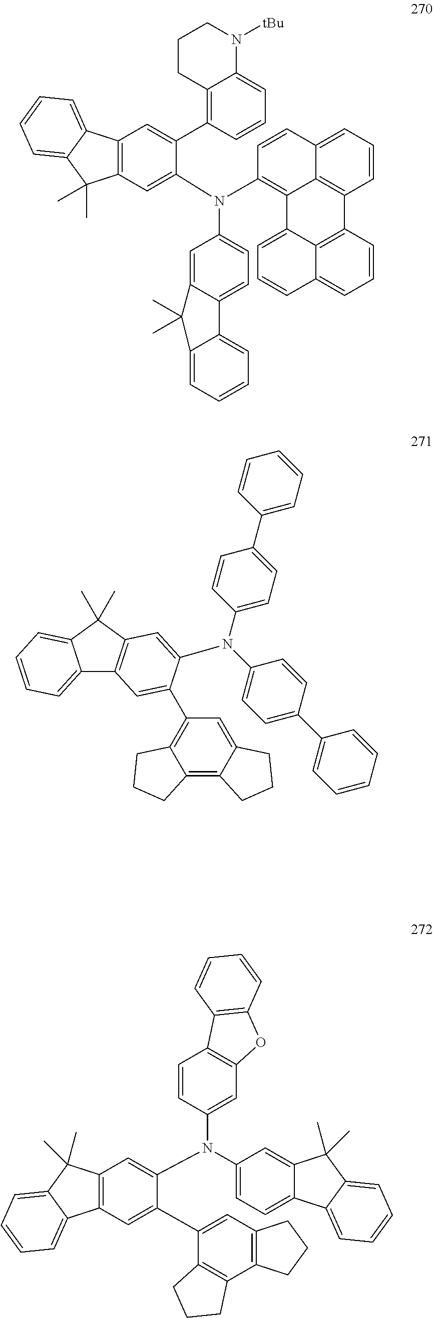 [SMILES: CC1(C)c2ccccc2-c2ccc(N(c3cc4c(cc3-c3cccc5c3CCCN5C(C)(C)C)-c3ccccc3C4(C)C)c3ccc4cccc5c6cccc7cccc(c3c45)c76)cc21 ; CC1(C)c2ccccc2-c2cc(-c3cc4c(c5c3CCC5)CCC4)c(N(c3ccc(-c4ccccc4)cc3)c3ccc(-c4ccccc4)cc3)cc21 ; CC1(C)c2ccccc2-c2ccc(N(c3ccc4c(c3)oc3ccccc34)c3cc4c(cc3-c3cc5c(c6c3CCC6)CCC5)-c3ccccc3C4(C)C)cc21]

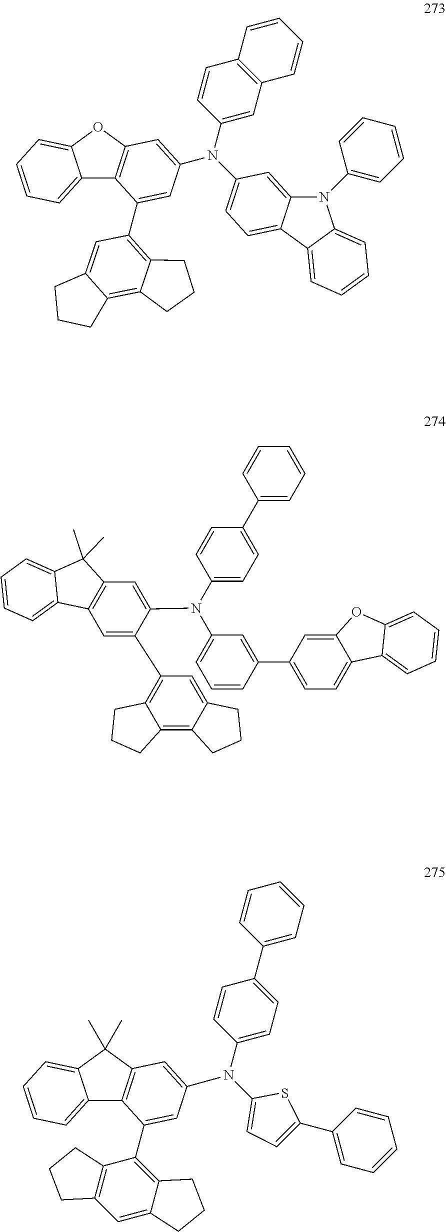 [SMILES: c1ccc(-n2c3ccccc3c3ccc(N(c4ccc5ccccc5c4)c4cc(-c5cc6c(c7c5CCC7)CCC6)c5c(c4)oc4ccccc45)cc32)cc1 ; CC1(C)c2ccccc2-c2cc(-c3cc4c(c5c3CCC5)CCC4)c(N(c3ccc(-c4ccccc4)cc3)c3cccc(-c4ccc5c(c4)oc4ccccc45)c3)cc21 ; CC1(C)c2ccccc2-c2c(-c3c4c(cc5c3CCC5)CCC4)cc(N(c3ccc(-c4ccccc4)cc3)c3ccc(-c4ccccc4)s3)cc21]

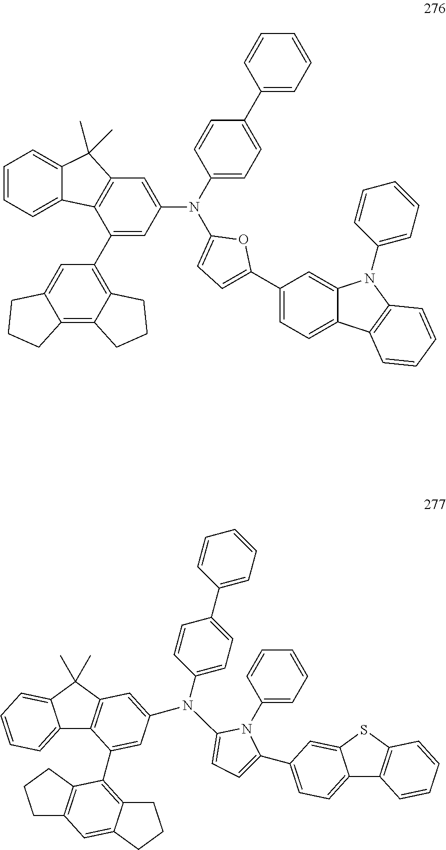 [SMILES: CC1(C)c2ccccc2-c2c(-c3cc4c(c5c3CCC5)CCC4)cc(N(c3ccc(-c4ccccc4)cc3)c3ccc(-c4ccc5c6ccccc6n(-c6ccccc6)c5c4)o3)cc21 ; CC1(C)c2ccccc2-c2c(-c3c4c(cc5c3CCC5)CCC4)cc(N(c3ccc(-c4ccccc4)cc3)c3ccc(-c4ccc5c(c4)sc4ccccc45)n3-c3ccccc3)cc21]

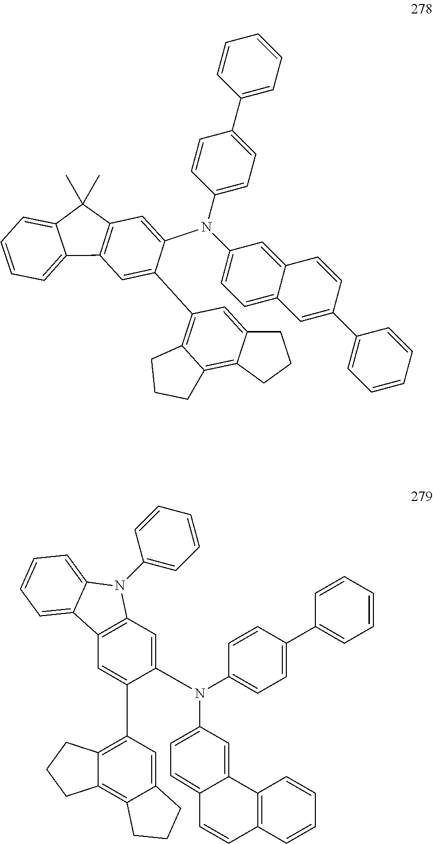 [SMILES: CC1(C)c2ccccc2-c2cc(-c3cc4c(c5c3CCC5)CCC4)c(N(c3ccc(-c4ccccc4)cc3)c3ccc4cc(-c5ccccc5)ccc4c3)cc21 ; c1ccc(-c2ccc(N(c3ccc4ccc5ccccc5c4c3)c3cc4c(cc3-c3cc5c(c6c3CCC6)CCC5)c3ccccc3n4-c3ccccc3)cc2)cc1]

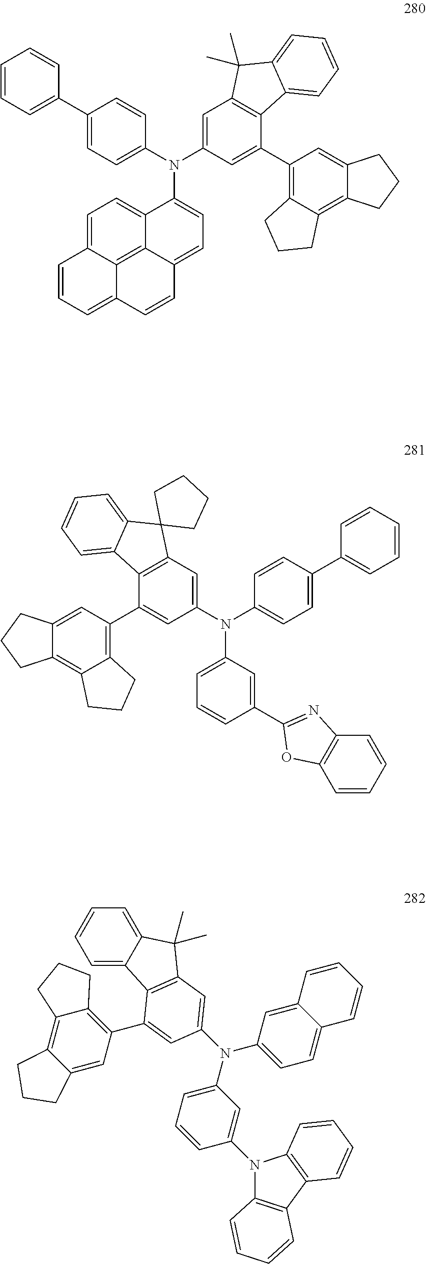 [SMILES: CC1(C)c2ccccc2-c2c(-c3cc4c(c5c3CCC5)CCC4)cc(N(c3ccc(-c4ccccc4)cc3)c3ccc4ccc5cccc6ccc3c4c56)cc21 ; c1ccc(-c2ccc(N(c3cccc(-c4nc5ccccc5o4)c3)c3cc(-c4cc5c(c6c4CCC6)CCC5)c4c(c3)C3(CCCC3)c3ccccc3-4)cc2)cc1 ; CC1(C)c2ccccc2-c2c(-c3cc4c(c5c3CCC5)CCC4)cc(N(c3cccc(-n4c5ccccc5c5ccccc54)c3)c3ccc4ccccc4c3)cc21]

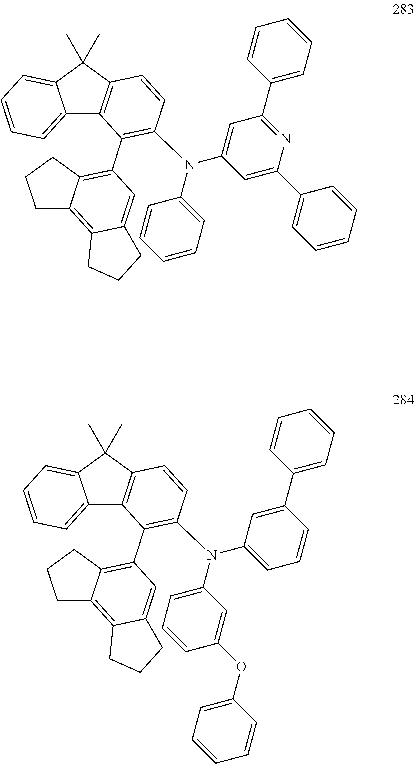 [SMILES: CC1(C)c2ccccc2-c2c1ccc(N(c1ccccc1)c1cc(-c3ccccc3)nc(-c3ccccc3)c1)c2-c1cc2c(c3c1CCC3)CCC2 ; CC1(C)c2ccccc2-c2c1ccc(N(c1cccc(Oc3ccccc3)c1)c1cccc(-c3ccccc3)c1)c2-c1cc2c(c3c1CCC3)CCC2]

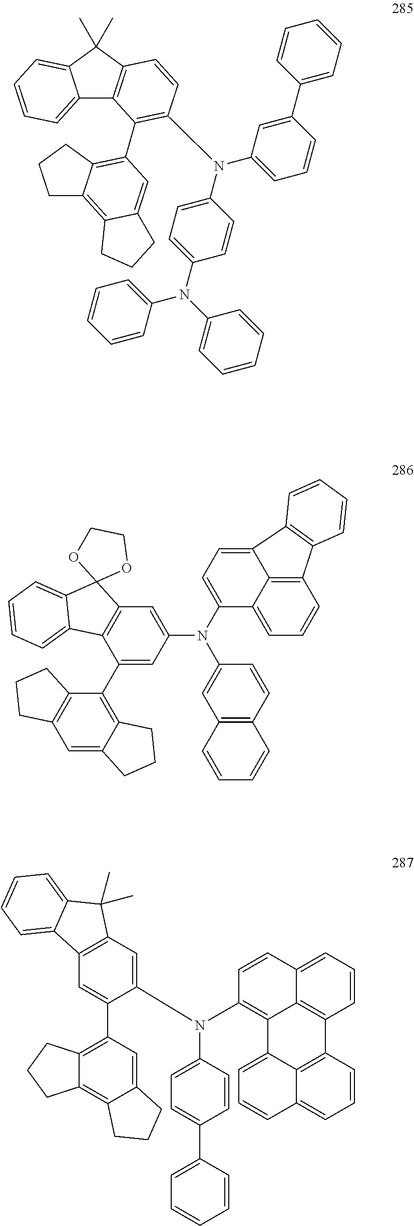 [SMILES: CC1(C)c2ccccc2-c2c1ccc(N(c1ccc(N(c3ccccc3)c3ccccc3)cc1)c1cccc(-c3ccccc3)c1)c2-c1cc2c(c3c1CCC3)CCC2 ; c1ccc2c(c1)-c1cccc3c(N(c4cc(-c5c6c(cc7c5CCC7)CCC6)c5c(c4)C4(OCCO4)c4ccccc4-5)c4ccc5ccccc5c4)ccc-2c13 ; CC1(C)c2ccccc2-c2cc(-c3cc4c(c5c3CCC5)CCC4)c(N(c3ccc(-c4ccccc4)cc3)c3ccc4cccc5c6cccc7cccc(c3c45)c76)cc21]

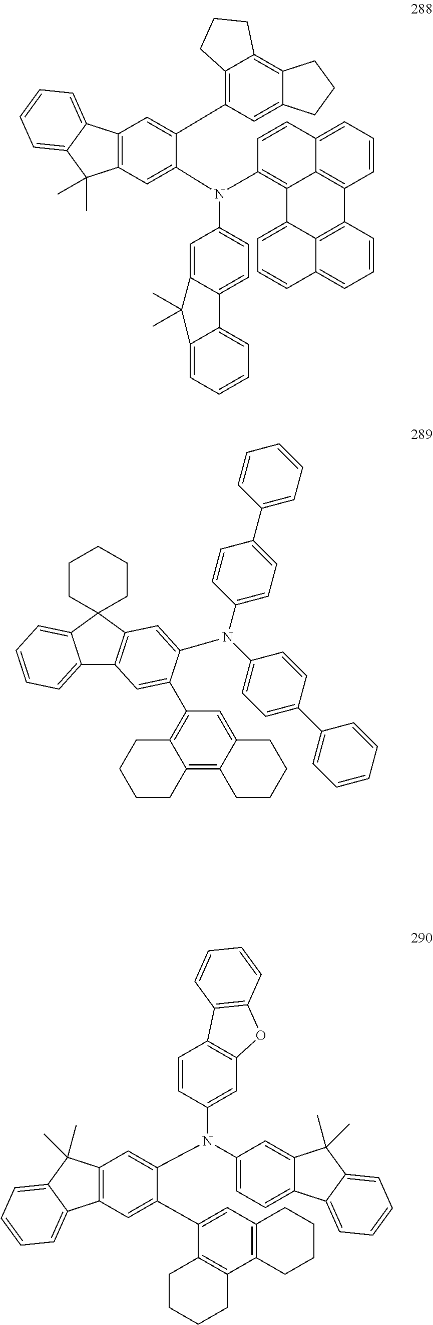 [SMILES: CC1(C)c2ccccc2-c2ccc(N(c3cc4c(cc3-c3cc5c(c6c3CCC6)CCC5)-c3ccccc3C4(C)C)c3ccc4cccc5c6cccc7cccc(c3c45)c76)cc21 ; c1ccc(-c2ccc(N(c3ccc(-c4ccccc4)cc3)c3cc4c(cc3-c3cc5c(c6c3CCCC6)CCCC5)-c3ccccc3C43CCCCC3)cc2)cc1 ; CC1(C)c2ccccc2-c2ccc(N(c3ccc4c(c3)oc3ccccc34)c3cc4c(cc3-c3cc5c(c6c3CCCC6)CCCC5)-c3ccccc3C4(C)C)cc21]

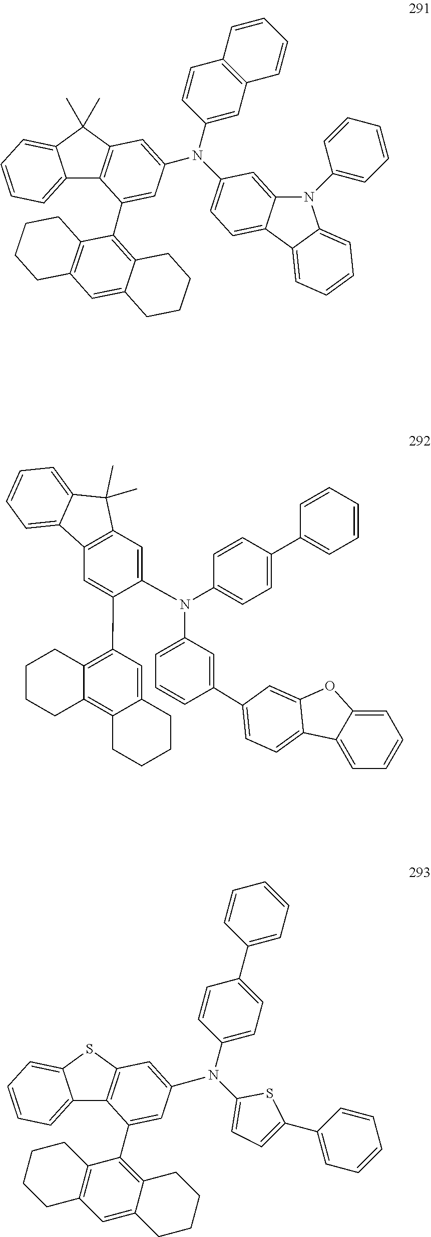 [SMILES: CC1(C)c2ccccc2-c2c(-c3c4c(cc5c3CCCC5)CCCC4)cc(N(c3ccc4ccccc4c3)c3ccc4c5ccccc5n(-c5ccccc5)c4c3)cc21 ; CC1(C)c2ccccc2-c2cc(-c3cc4c(c5c3CCCC5)CCCC4)c(N(c3ccc(-c4ccccc4)cc3)c3cccc(-c4ccc5c(c4)oc4ccccc45)c3)cc21 ; c1ccc(-c2ccc(N(c3cc(-c4c5c(cc6c4CCCC6)CCCC5)c4c(c3)sc3ccccc34)c3ccc(-c4ccccc4)s3)cc2)cc1]

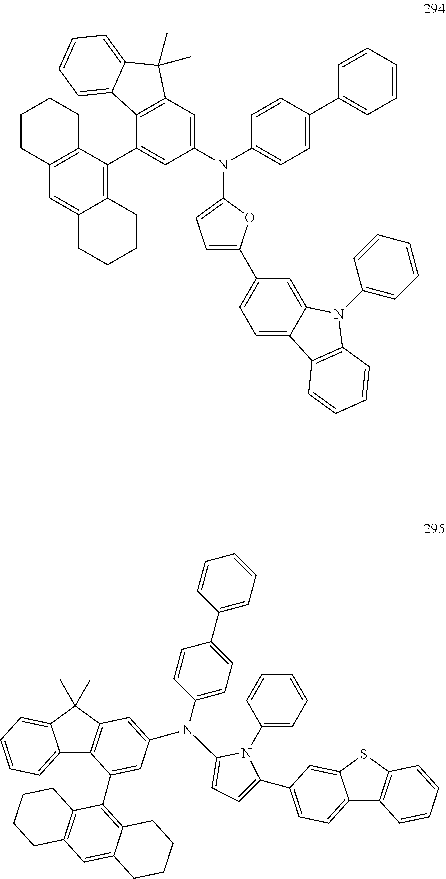 [SMILES: CC1(C)c2ccccc2-c2c(-c3c4c(cc5c3CCCC5)CCCC4)cc(N(c3ccc(-c4ccccc4)cc3)c3ccc(-c4ccc5c6ccccc6n(-c6ccccc6)c5c4)o3)cc21 ; CC1(C)c2ccccc2-c2c(-c3c4c(cc5c3CCCC5)CCCC4)cc(N(c3ccc(-c4ccccc4)cc3)c3ccc(-c4ccc5c(c4)sc4ccccc45)n3-c3ccccc3)cc21]

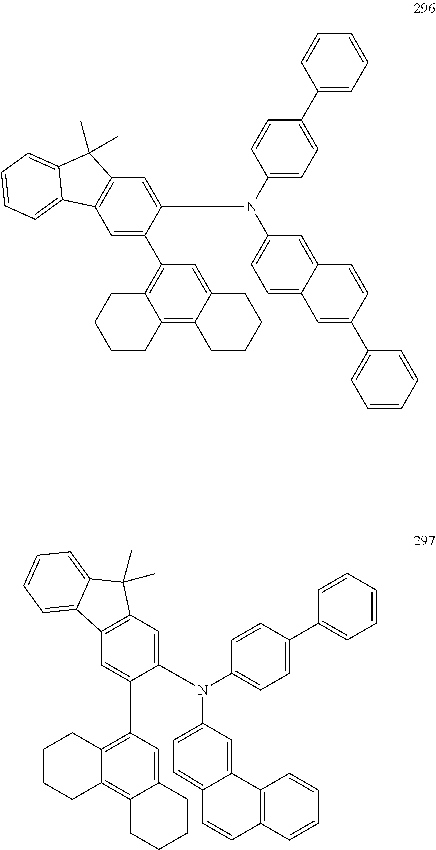 [SMILES: CC1(C)c2ccccc2-c2cc(-c3cc4c(c5c3CCCC5)CCCC4)c(N(c3ccc(-c4ccccc4)cc3)c3ccc4cc(-c5ccccc5)ccc4c3)cc21 ; CC1(C)c2ccccc2-c2cc(-c3cc4c(c5c3CCCC5)CCCC4)c(N(c3ccc(-c4ccccc4)cc3)c3ccc4ccc5ccccc5c4c3)cc21]

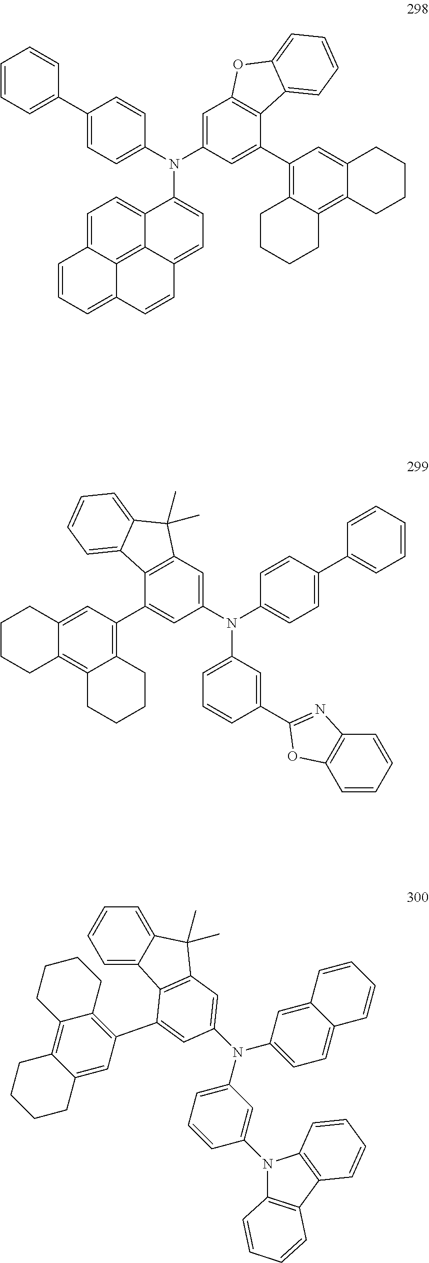 [SMILES: c1ccc(-c2ccc(N(c3cc(-c4cc5c(c6c4CCCC6)CCCC5)c4c(c3)oc3ccccc34)c3ccc4ccc5cccc6ccc3c4c56)cc2)cc1 ; CC1(C)c2ccccc2-c2c(-c3cc4c(c5c3CCCC5)CCCC4)cc(N(c3ccc(-c4ccccc4)cc3)c3cccc(-c4nc5ccccc5o4)c3)cc21 ; CC1(C)c2ccccc2-c2c(-c3cc4c(c5c3CCCC5)CCCC4)cc(N(c3cccc(-n4c5ccccc5c5ccccc54)c3)c3ccc4ccccc4c3)cc21]

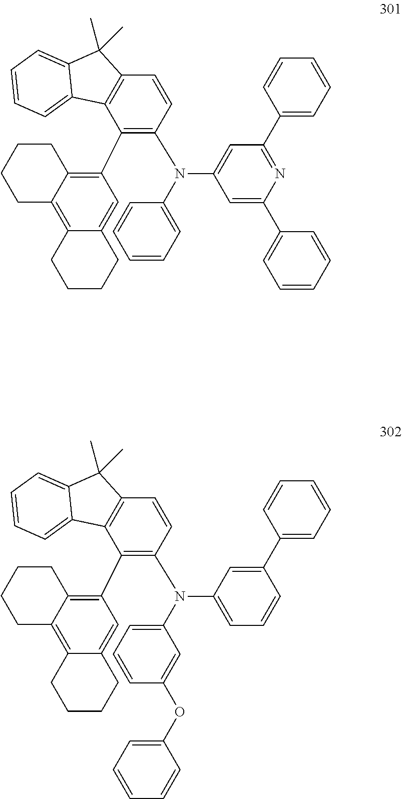 [SMILES: CC1(C)c2ccccc2-c2c1ccc(N(c1ccccc1)c1cc(-c3ccccc3)nc(-c3ccccc3)c1)c2-c1cc2c(c3c1CCCC3)CCCC2 ; CC1(C)c2ccccc2-c2c1ccc(N(c1cccc(Oc3ccccc3)c1)c1cccc(-c3ccccc3)c1)c2-c1cc2c(c3c1CCCC3)CCCC2]

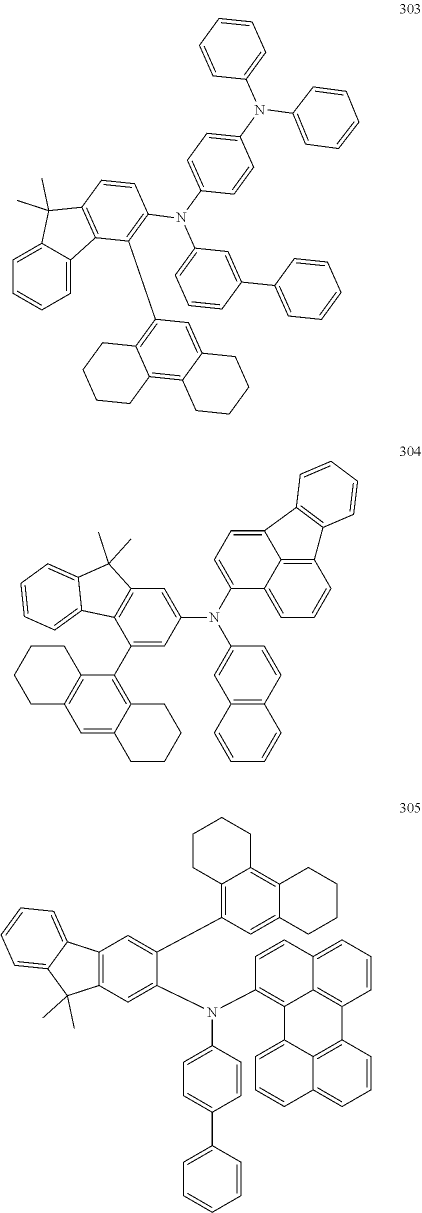 [SMILES: CC1(C)c2ccccc2-c2c1ccc(N(c1ccc(N(c3ccccc3)c3ccccc3)cc1)c1cccc(-c3ccccc3)c1)c2-c1cc2c(c3c1CCCC3)CCCC2 ; CC1(C)c2ccccc2-c2c(-c3c4c(cc5c3CCCC5)CCCC4)cc(N(c3ccc4ccccc4c3)c3ccc4c5c(cccc35)-c3ccccc3-4)cc21 ; CC1(C)c2ccccc2-c2cc(-c3cc4c(c5c3CCCC5)CCCC4)c(N(c3ccc(-c4ccccc4)cc3)c3ccc4cccc5c6cccc7cccc(c3c45)c76)cc21]

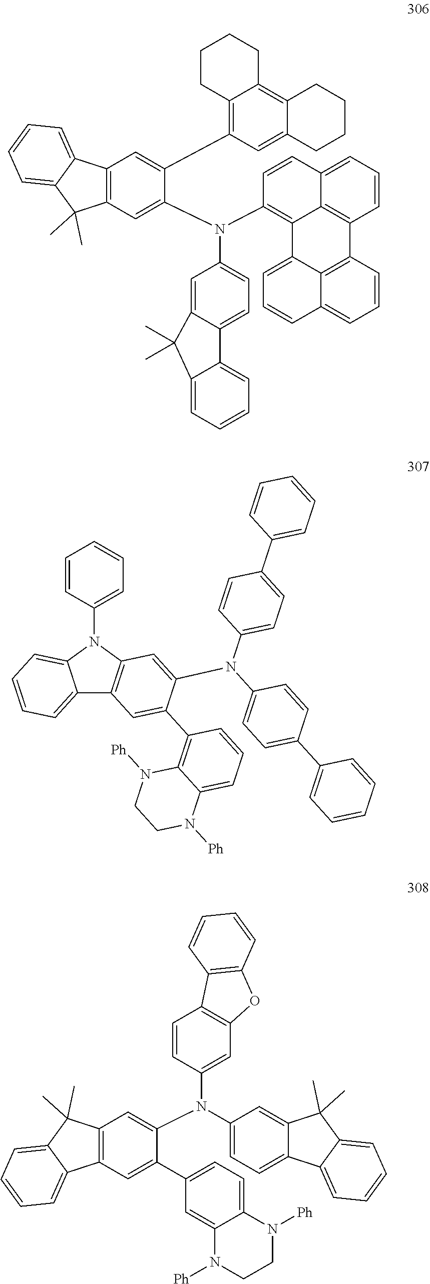 [SMILES: CC1(C)c2ccccc2-c2ccc(N(c3cc4c(cc3-c3cc5c(c6c3CCCC6)CCCC5)-c3ccccc3C4(C)C)c3ccc4cccc5c6cccc7cccc(c3c45)c76)cc21 ; c1ccc(-c2ccc(N(c3ccc(-c4ccccc4)cc3)c3cc4c(cc3-c3cccc5c3N(c3ccccc3)CCN5c3ccccc3)c3ccccc3n4-c3ccccc3)cc2)cc1 ; CC1(C)c2ccccc2-c2ccc(N(c3ccc4c(c3)oc3ccccc34)c3cc4c(cc3-c3ccc5c(c3)N(c3ccccc3)CCN5c3ccccc3)-c3ccccc3C4(C)C)cc21]

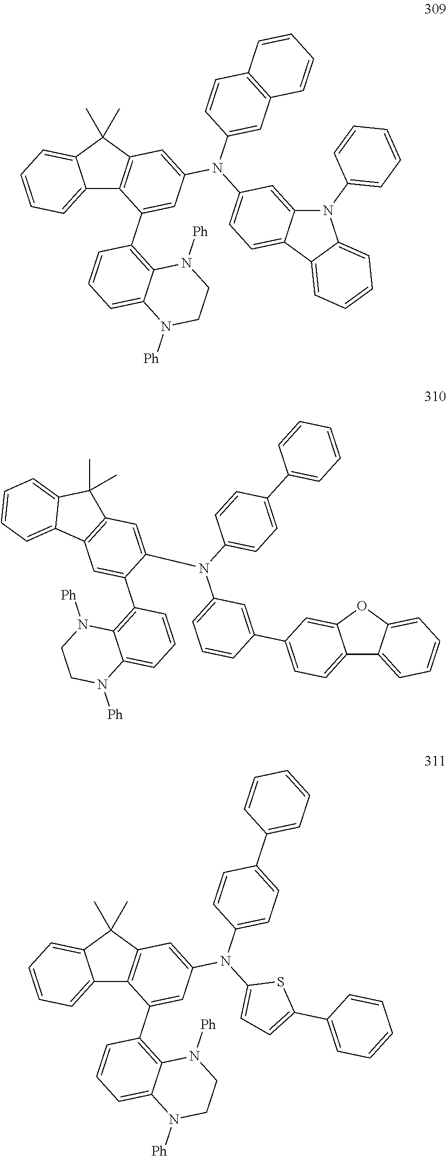 [SMILES: CC1(C)c2ccccc2-c2c(-c3cccc4c3N(c3ccccc3)CCN4c3ccccc3)cc(N(c3ccc4ccccc4c3)c3ccc4c5ccccc5n(-c5ccccc5)c4c3)cc21 ; CC1(C)c2ccccc2-c2cc(-c3cccc4c3N(c3ccccc3)CCN4c3ccccc3)c(N(c3ccc(-c4ccccc4)cc3)c3cccc(-c4ccc5c(c4)oc4ccccc45)c3)cc21 ; CC1(C)c2ccccc2-c2c(-c3cccc4c3N(c3ccccc3)CCN4c3ccccc3)cc(N(c3ccc(-c4ccccc4)cc3)c3ccc(-c4ccccc4)s3)cc21]

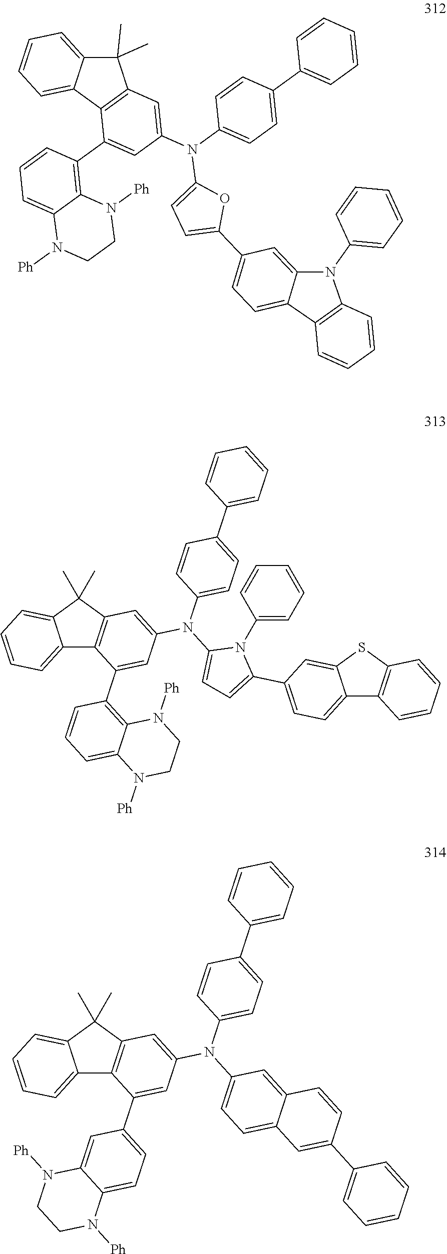 [SMILES: CC1(C)c2ccccc2-c2c(-c3cccc4c3N(c3ccccc3)CCN4c3ccccc3)cc(N(c3ccc(-c4ccccc4)cc3)c3ccc(-c4ccc5c6ccccc6n(-c6ccccc6)c5c4)o3)cc21 ; CC1(C)c2ccccc2-c2c(-c3cccc4c3N(c3ccccc3)CCN4c3ccccc3)cc(N(c3ccc(-c4ccccc4)cc3)c3ccc(-c4ccc5c(c4)sc4ccccc45)n3-c3ccccc3)cc21 ; CC1(C)c2ccccc2-c2c(-c3ccc4c(c3)N(c3ccccc3)CCN4c3ccccc3)cc(N(c3ccc(-c4ccccc4)cc3)c3ccc4cc(-c5ccccc5)ccc4c3)cc21]

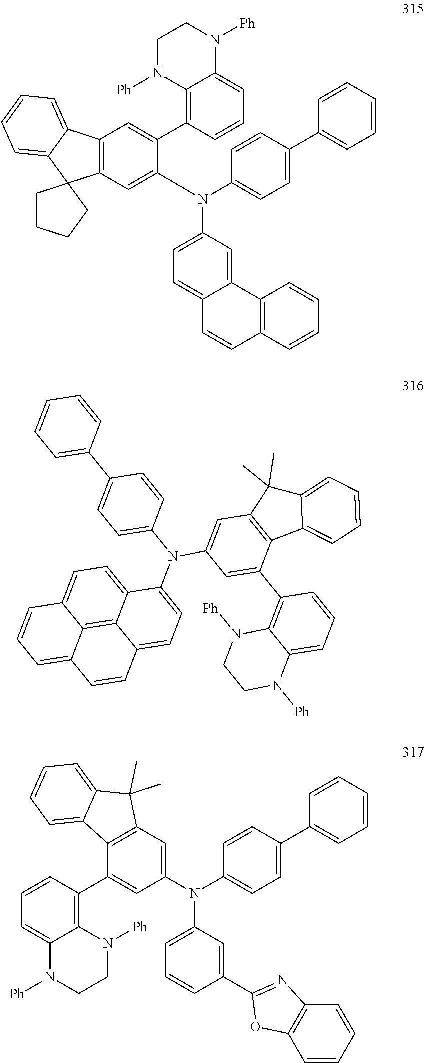 [SMILES: c1ccc(-c2ccc(N(c3ccc4ccc5ccccc5c4c3)c3cc4c(cc3-c3cccc5c3N(c3ccccc3)CCN5c3ccccc3)-c3ccccc3C43CCCC3)cc2)cc1 ; CC1(C)c2ccccc2-c2c(-c3cccc4c3N(c3ccccc3)CCN4c3ccccc3)cc(N(c3ccc(-c4ccccc4)cc3)c3ccc4ccc5cccc6ccc3c4c56)cc21 ; CC1(C)c2ccccc2-c2c(-c3cccc4c3N(c3ccccc3)CCN4c3ccccc3)cc(N(c3ccc(-c4ccccc4)cc3)c3cccc(-c4nc5ccccc5o4)c3)cc21]

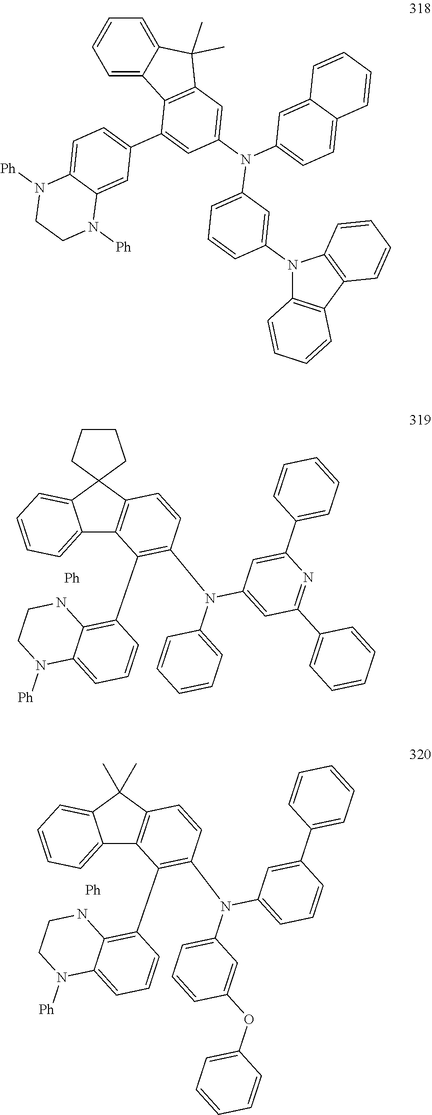 [SMILES: CC1(C)c2ccccc2-c2c(-c3ccc4c(c3)N(c3ccccc3)CCN4c3ccccc3)cc(N(c3cccc(-n4c5ccccc5c5ccccc54)c3)c3ccc4ccccc4c3)cc21 ; c1ccc(-c2cc(N(c3ccccc3)c3ccc4c(c3-c3cccc5c3NCCN5c3ccccc3)-c3ccccc3C43CCCC3)cc(-c3ccccc3)n2)cc1 ; "Ph" ; CC1(C)c2ccccc2-c2c1ccc(N(c1cccc(Oc3ccccc3)c1)c1cccc(-c3ccccc3)c1)c2-c1cccc2c1NCCN2c1ccccc1 ; "Ph"]

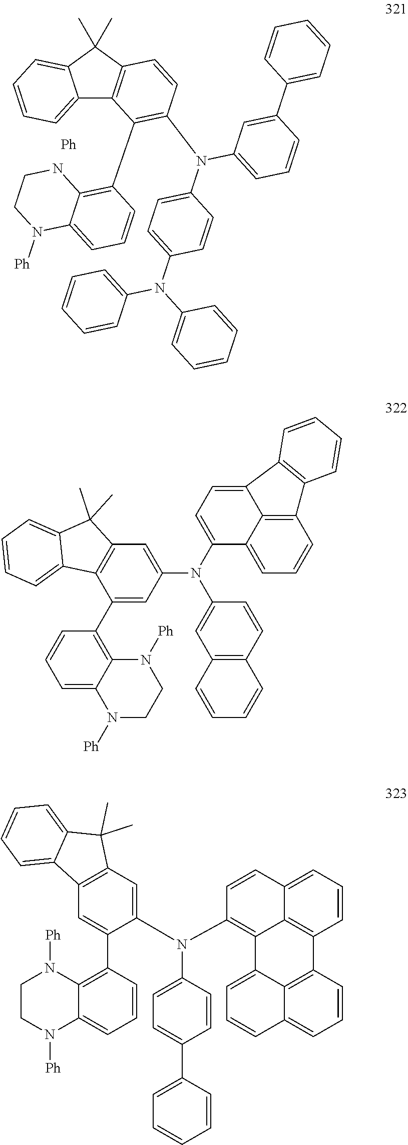 [SMILES: CC1(C)c2ccccc2-c2c1ccc(N(c1ccc(N(c3ccccc3)c3ccccc3)cc1)c1cccc(-c3ccccc3)c1)c2-c1cccc2c1NCCN2c1ccccc1 ; "Ph" ; CC1(C)c2ccccc2-c2c(-c3cccc4c3N(c3ccccc3)CCN4c3ccccc3)cc(N(c3ccc4ccccc4c3)c3ccc4c5c(cccc35)-c3ccccc3-4)cc21 ; CC1(C)c2ccccc2-c2cc(-c3cccc4c3N(c3ccccc3)CCN4c3ccccc3)c(N(c3ccc(-c4ccccc4)cc3)c3ccc4cccc5c6cccc7cccc(c3c45)c76)cc21]

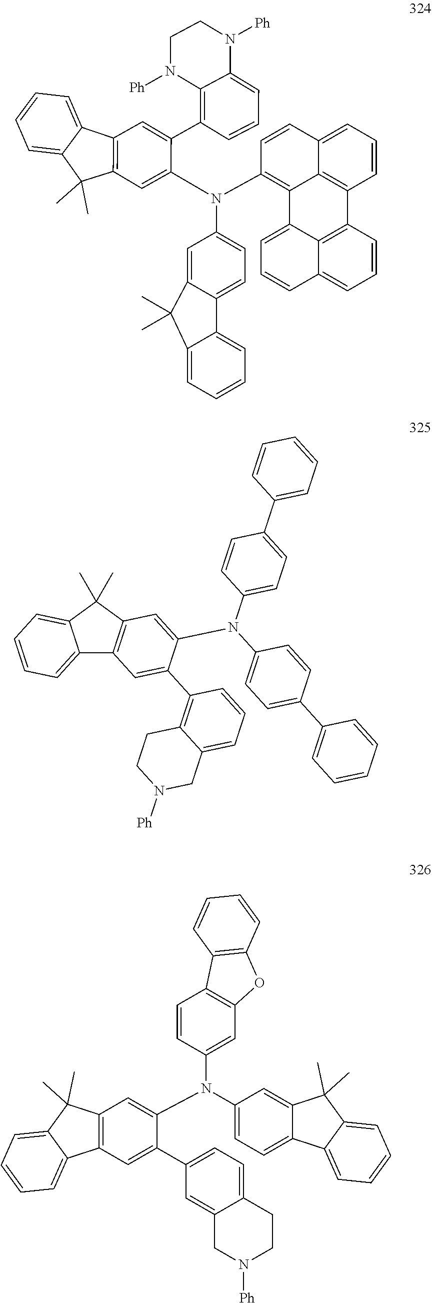 [SMILES: CC1(C)c2ccccc2-c2ccc(N(c3cc4c(cc3-c3cccc5c3N(c3ccccc3)CCN5c3ccccc3)-c3ccccc3C4(C)C)c3ccc4cccc5c6cccc7cccc(c3c45)c76)cc21 ; CC1(C)c2ccccc2-c2cc(-c3cccc4c3CCN(c3ccccc3)C4)c(N(c3ccc(-c4ccccc4)cc3)c3ccc(-c4ccccc4)cc3)cc21 ; CC1(C)c2ccccc2-c2ccc(N(c3ccc4c(c3)oc3ccccc34)c3cc4c(cc3-c3ccc5c(c3)CN(c3ccccc3)CC5)-c3ccccc3C4(C)C)cc21]

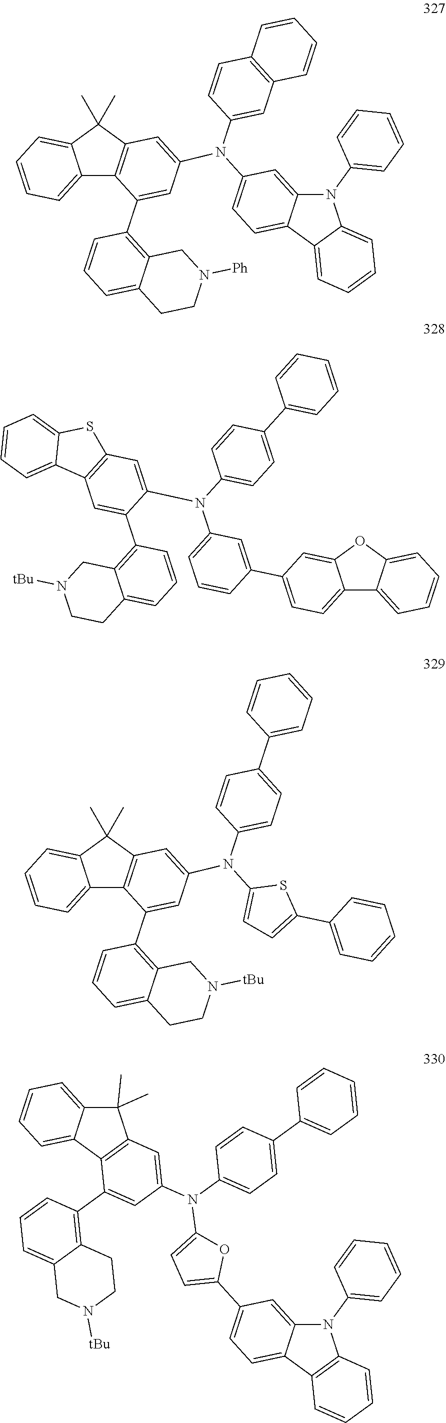 [SMILES: CC1(C)c2ccccc2-c2c(-c3cccc4c3CN(c3ccccc3)CC4)cc(N(c3ccc4ccccc4c3)c3ccc4c5ccccc5n(-c5ccccc5)c4c3)cc21 ; CC(C)(C)N1CCc2cccc(-c3cc4c(cc3N(c3ccc(-c5ccccc5)cc3)c3cccc(-c5ccc6c(c5)oc5ccccc56)c3)sc3ccccc34)c2C1 ; CC1(C)c2ccccc2-c2c(-c3cccc4c3CN(C(C)(C)C)CC4)cc(N(c3ccc(-c4ccccc4)cc3)c3ccc(-c4ccccc4)s3)cc21 ; CC1(C)c2ccccc2-c2c(-c3cccc4c3CCN(C(C)(C)C)C4)cc(N(c3ccc(-c4ccccc4)cc3)c3ccc(-c4ccc5c6ccccc6n(-c6ccccc6)c5c4)o3)cc21]

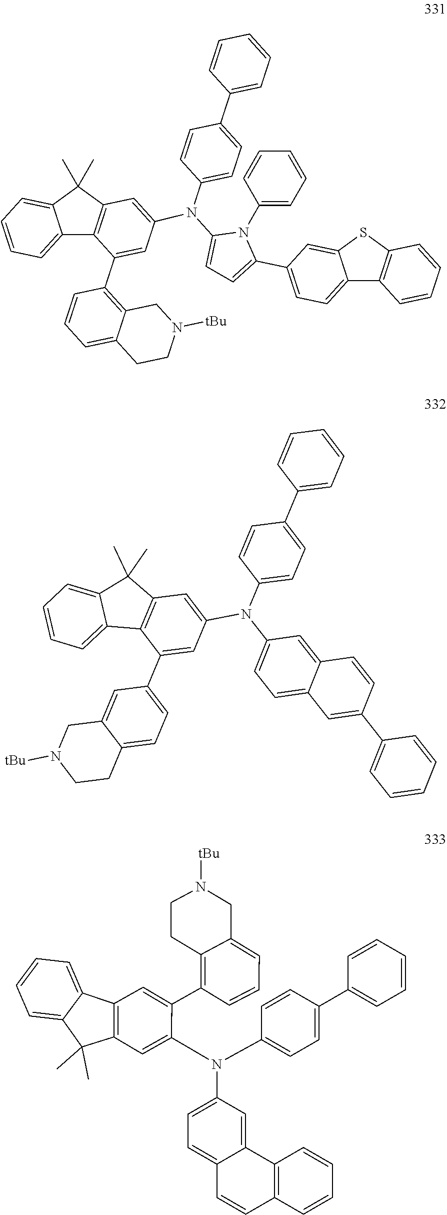 [SMILES: CC1(C)c2ccccc2-c2c(-c3cccc4c3CN(C(C)(C)C)CC4)cc(N(c3ccc(-c4ccccc4)cc3)c3ccc(-c4ccc5c(c4)sc4ccccc45)n3-c3ccccc3)cc21 ; CC1(C)c2ccccc2-c2c(-c3ccc4c(c3)CN(C(C)(C)C)CC4)cc(N(c3ccc(-c4ccccc4)cc3)c3ccc4cc(-c5ccccc5)ccc4c3)cc21 ; CC1(C)c2ccccc2-c2cc(-c3cccc4c3CCN(C(C)(C)C)C4)c(N(c3ccc(-c4ccccc4)cc3)c3ccc4ccc5ccccc5c4c3)cc21]

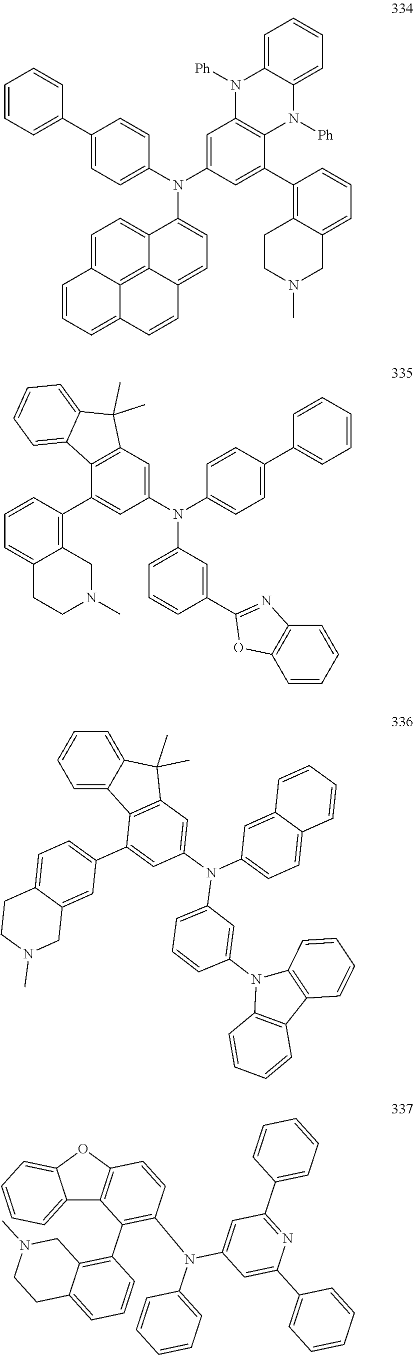 [SMILES: CN1CCc2c(cccc2-c2cc(N(c3ccc(-c4ccccc4)cc3)c3ccc4ccc5cccc6ccc3c4c56)cc3c2N(c2ccccc2)c2ccccc2N3c2ccccc2)C1 ; CN1CCc2cccc(-c3cc(N(c4ccc(-c5ccccc5)cc4)c4cccc(-c5nc6ccccc6o5)c4)cc4c3-c3ccccc3C4(C)C)c2C1 ; CN1CCc2ccc(-c3cc(N(c4cccc(-n5c6ccccc6c6ccccc65)c4)c4ccc5ccccc5c4)cc4c3-c3ccccc3C4(C)C)cc2C1 ; CN1CCc2cccc(-c3c(N(c4ccccc4)c4cc(-c5ccccc5)nc(-c5ccccc5)c4)ccc4oc5ccccc5c34)c2C1]

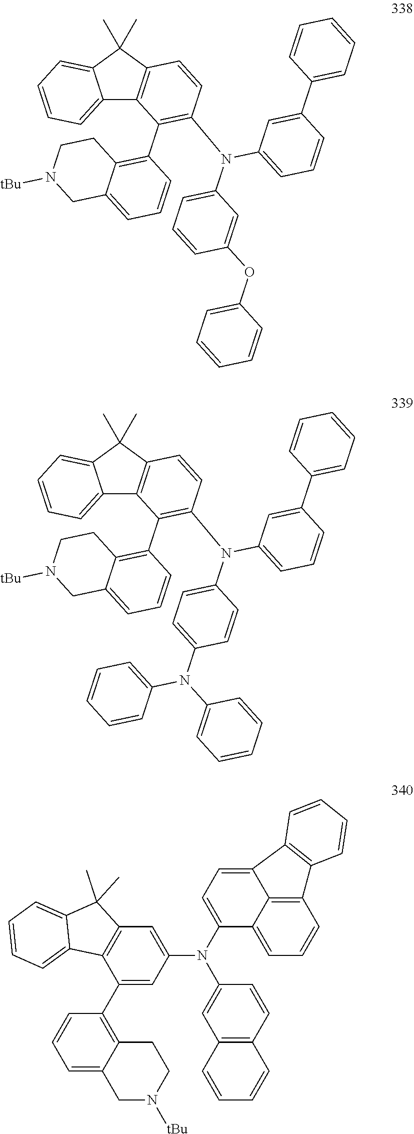 [SMILES: CC1(C)c2ccccc2-c2c1ccc(N(c1cccc(Oc3ccccc3)c1)c1cccc(-c3ccccc3)c1)c2-c1cccc2c1CCN(C(C)(C)C)C2 ; CC1(C)c2ccccc2-c2c1ccc(N(c1ccc(N(c3ccccc3)c3ccccc3)cc1)c1cccc(-c3ccccc3)c1)c2-c1cccc2c1CCN(C(C)(C)C)C2 ; CC1(C)c2ccccc2-c2c(-c3cccc4c3CCN(C(C)(C)C)C4)cc(N(c3ccc4ccccc4c3)c3ccc4c5c(cccc35)-c3ccccc3-4)cc21]

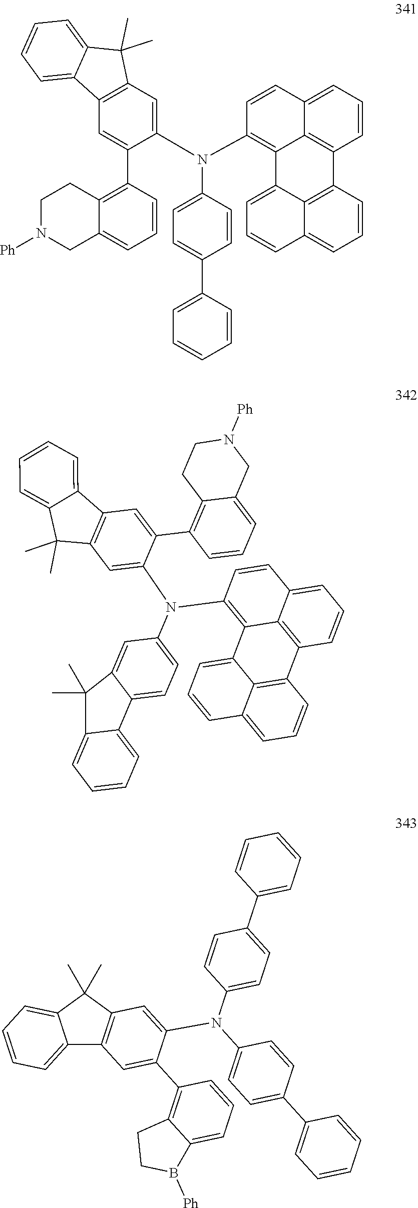 [SMILES: CC1(C)c2ccccc2-c2cc(-c3cccc4c3CCN(c3ccccc3)C4)c(N(c3ccc(-c4ccccc4)cc3)c3ccc4cccc5c6cccc7cccc(c3c45)c76)cc21 ; CC1(C)c2ccccc2-c2ccc(N(c3cc4c(cc3-c3cccc5c3CCN(c3ccccc3)C5)-c3ccccc3C4(C)C)c3ccc4cccc5c6cccc7cccc(c3c45)c76)cc21 ; CC1(C)c2ccccc2-c2cc(-c3cccc4c3CCB4c3ccccc3)c(N(c3ccc(-c4ccccc4)cc3)c3ccc(-c4ccccc4)cc3)cc21]

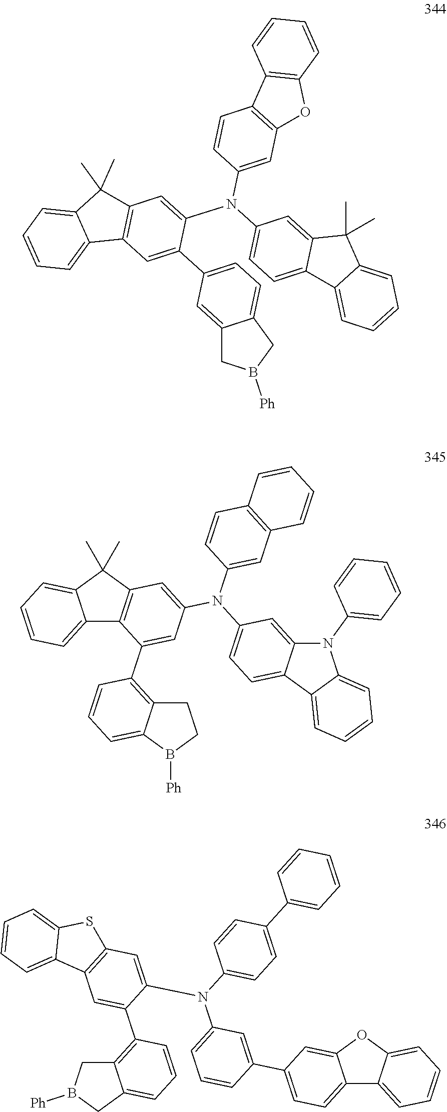 [SMILES: CC1(C)c2ccccc2-c2ccc(N(c3ccc4c(c3)oc3ccccc34)c3cc4c(cc3-c3ccc5c(c3)CB(c3ccccc3)C5)-c3ccccc3C4(C)C)cc21 ; CC1(C)c2ccccc2-c2c(-c3cccc4c3CCB4c3ccccc3)cc(N(c3ccc4ccccc4c3)c3ccc4c5ccccc5n(-c5ccccc5)c4c3)cc21 ; c1ccc(B2Cc3cccc(-c4cc5c(cc4N(c4ccc(-c6ccccc6)cc4)c4cccc(-c6ccc7c(c6)oc6ccccc67)c4)sc4ccccc45)c3C2)cc1]

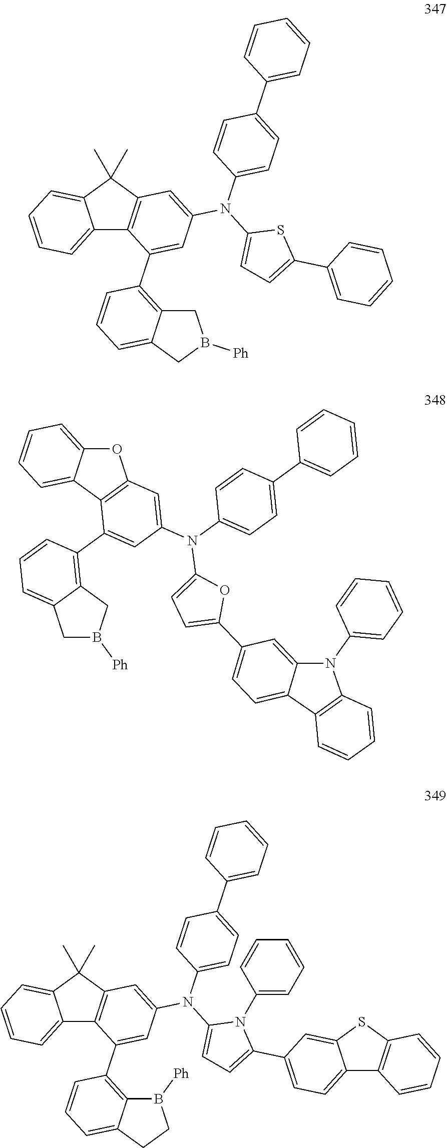 [SMILES: CC1(C)c2ccccc2-c2c(-c3cccc4c3CB(c3ccccc3)C4)cc(N(c3ccc(-c4ccccc4)cc3)c3ccc(-c4ccccc4)s3)cc21 ; c1ccc(B2Cc3cccc(-c4cc(N(c5ccc(-c6ccccc6)cc5)c5ccc(-c6ccc7c8ccccc8n(-c8ccccc8)c7c6)o5)cc5oc6ccccc6c45)c3C2)cc1 ; CC1(C)c2ccccc2-c2c(-c3cccc4c3B(c3ccccc3)CC4)cc(N(c3ccc(-c4ccccc4)cc3)c3ccc(-c4ccc5c(c4)sc4ccccc45)n3-c3ccccc3)cc21]

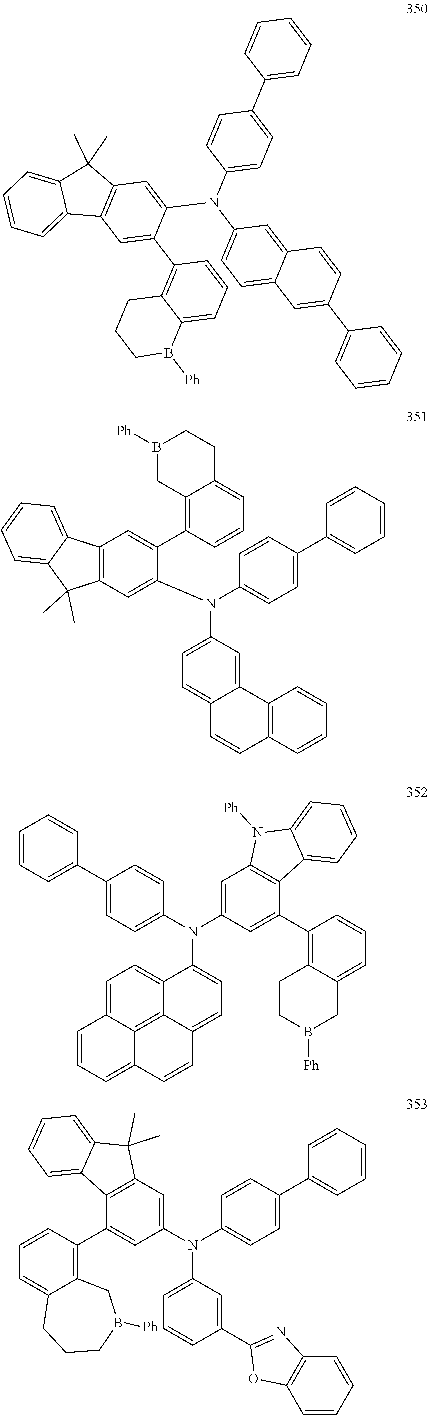

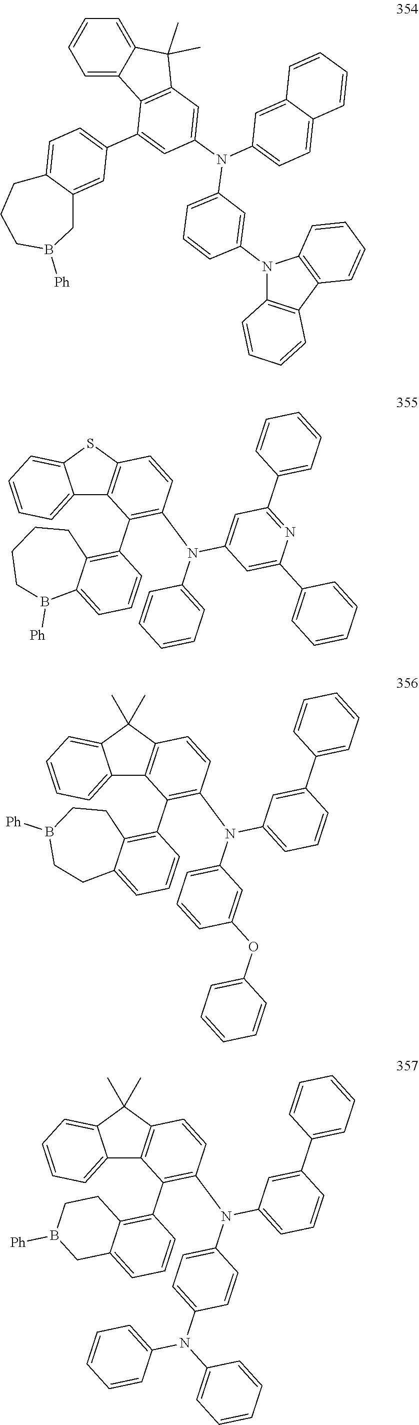 [SMILES: CC1(C)c2ccccc2-c2c(-c3ccc4c(c3)CB(c3ccccc3)CCC4)cc(N(c3cccc(-n4c5ccccc5c5ccccc54)c3)c3ccc4ccccc4c3)cc21 ; c1ccc(B2CCCCc3c2cccc3-c2c(N(c3ccccc3)c3cc(-c4ccccc4)nc(-c4ccccc4)c3)ccc3sc4ccccc4c23)cc1 ; CC1(C)c2ccccc2-c2c1ccc(N(c1cccc(Oc3ccccc3)c1)c1cccc(-c3ccccc3)c1)c2-c1cccc2c1CCB(c1ccccc1)CC2 ; CC1(C)c2ccccc2-c2c1ccc(N(c1ccc(N(c3ccccc3)c3ccccc3)cc1)c1cccc(-c3ccccc3)c1)c2-c1cccc2c1CCB(c1ccccc1)C2]

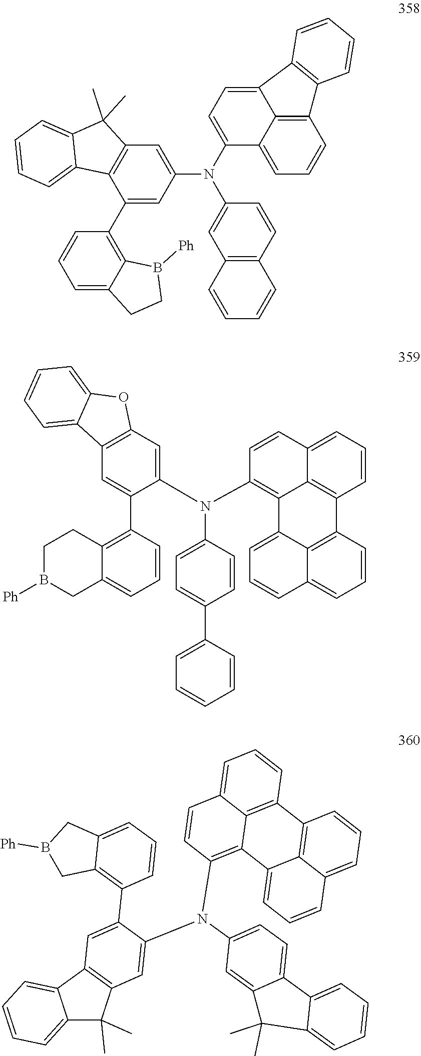 [SMILES: CC1(C)c2ccccc2-c2c(-c3cccc4c3B(c3ccccc3)CC4)cc(N(c3ccc4ccccc4c3)c3ccc4c5c(cccc35)-c3ccccc3-4)cc21 ; c1ccc(B2CCc3c(cccc3-c3cc4c(cc3N(c3ccc(-c5ccccc5)cc3)c3ccc5cccc6c7cccc8cccc(c3c56)c87)oc3ccccc34)C2)cc1 ; CC1(C)c2ccccc2-c2ccc(N(c3cc4c(cc3-c3cccc5c3CB(c3ccccc3)C5)-c3ccccc3C4(C)C)c3ccc4cccc5c6cccc7cccc(c3c45)c76)cc21]

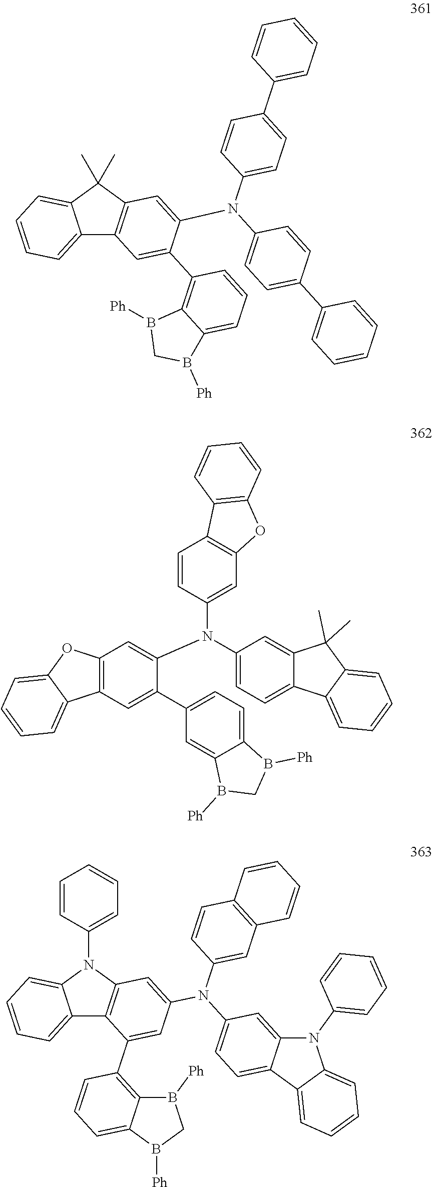 [SMILES: CC1(C)c2ccccc2-c2cc(-c3cccc4c3B(c3ccccc3)CB4c3ccccc3)c(N(c3ccc(-c4ccccc4)cc3)c3ccc(-c4ccccc4)cc3)cc21 ; CC1(C)c2ccccc2-c2ccc(N(c3ccc4c(c3)oc3ccccc34)c3cc4oc5ccccc5c4cc3-c3ccc4c(c3)B(c3ccccc3)CB4c3ccccc3)cc21 ; c1ccc(B2CB(c3ccccc3)c3c2cccc3-c2cc(N(c3ccc4ccccc4c3)c3ccc4c5ccccc5n(-c5ccccc5)c4c3)cc3c2c2ccccc2n3-c2ccccc2)cc1]

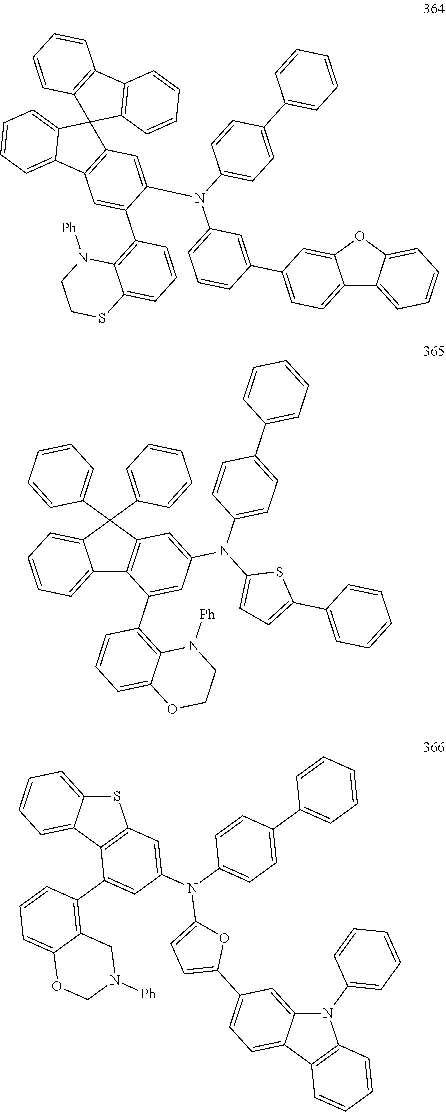 [SMILES: c1ccc(-c2ccc(N(c3cccc(-c4ccc5c(c4)oc4ccccc45)c3)c3cc4c(cc3-c3cccc5c3N(c3ccccc3)CCS5)-c3ccccc3C43c4ccccc4-c4ccccc43)cc2)cc1 ; c1ccc(-c2ccc(N(c3cc(-c4cccc5c4N(c4ccccc4)CCO5)c4c(c3)C(c3ccccc3)(c3ccccc3)c3ccccc3-4)c3ccc(-c4ccccc4)s3)cc2)cc1 ; c1ccc(-c2ccc(N(c3cc(-c4cccc5c4CN(c4ccccc4)CO5)c4c(c3)sc3ccccc34)c3ccc(-c4ccc5c6ccccc6n(-c6ccccc6)c5c4)o3)cc2)cc1]

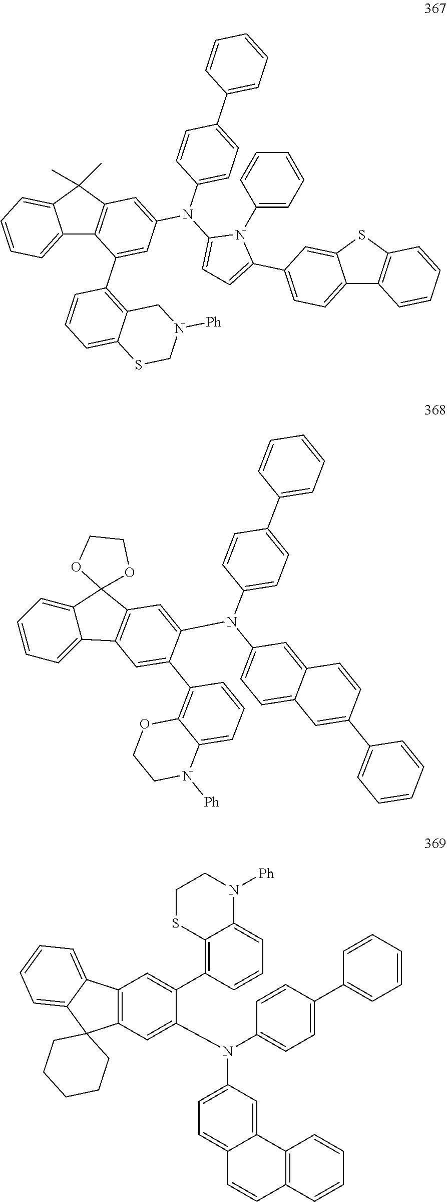 [SMILES: CC1(C)c2ccccc2-c2c(-c3cccc4c3CN(c3ccccc3)CS4)cc(N(c3ccc(-c4ccccc4)cc3)c3ccc(-c4ccc5c(c4)sc4ccccc45)n3-c3ccccc3)cc21 ; c1ccc(-c2ccc(N(c3ccc4cc(-c5ccccc5)ccc4c3)c3cc4c(cc3-c3cccc5c3OCCN5c3ccccc3)-c3ccccc3C43OCCO3)cc2)cc1 ; c1ccc(-c2ccc(N(c3ccc4ccc5ccccc5c4c3)c3cc4c(cc3-c3cccc5c3SCCN5c3ccccc3)-c3ccccc3C43CCCCC3)cc2)cc1]

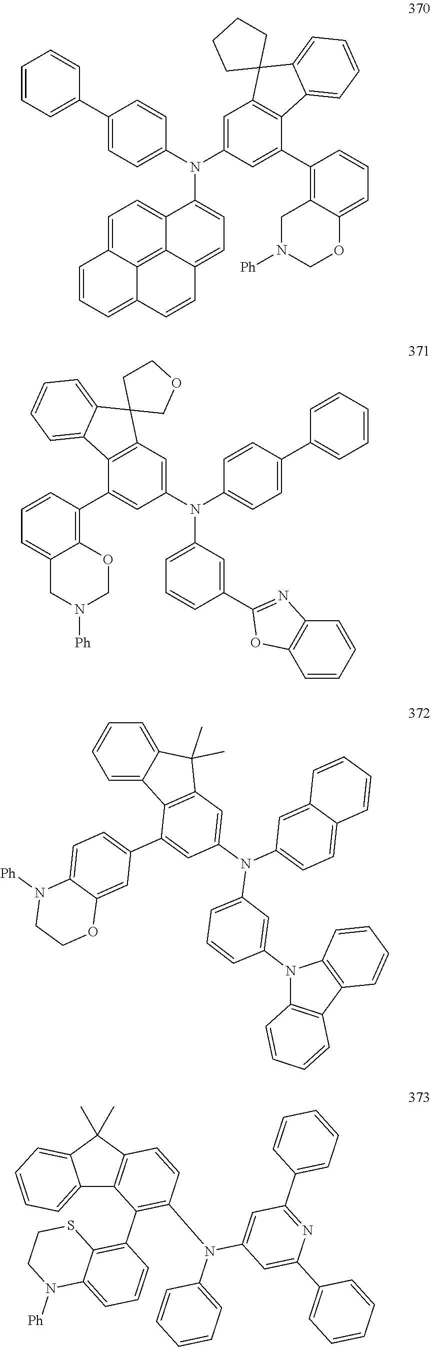

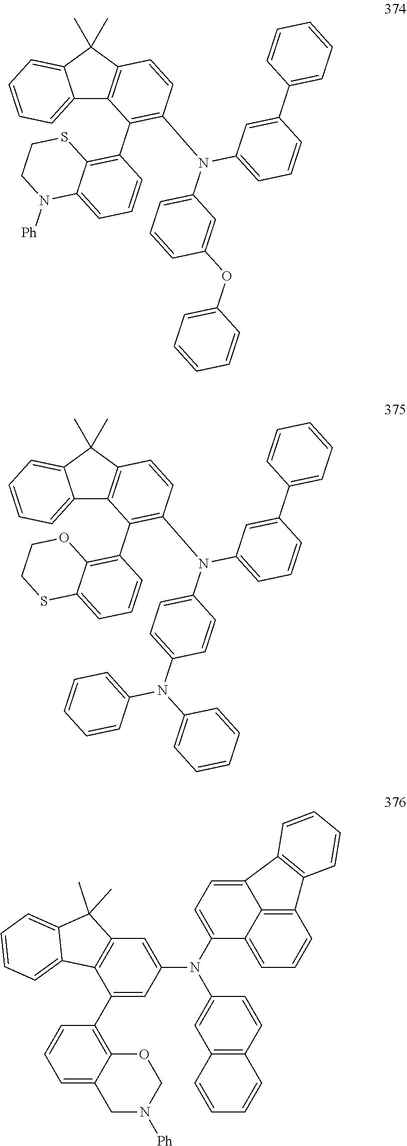 [SMILES: CC1(C)c2ccccc2-c2c1ccc(N(c1cccc(Oc3ccccc3)c1)c1cccc(-c3ccccc3)c1)c2-c1cccc2c1SCCN2c1ccccc1 ; CC1(C)c2ccccc2-c2c1ccc(N(c1ccc(N(c3ccccc3)c3ccccc3)cc1)c1cccc(-c3ccccc3)c1)c2-c1cccc2c1OCCS2 ; CC1(C)c2ccccc2-c2c(-c3cccc4c3OCN(c3ccccc3)C4)cc(N(c3ccc4ccccc4c3)c3ccc4c5c(cccc35)-c3ccccc3-4)cc21]

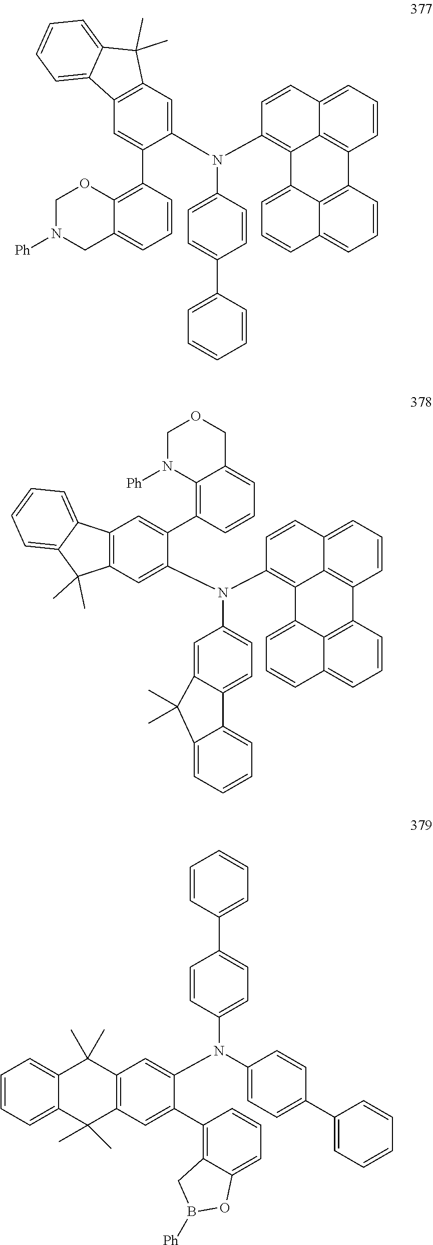 [SMILES: CC1(C)c2ccccc2-c2cc(-c3cccc4c3OCN(c3ccccc3)C4)c(N(c3ccc(-c4ccccc4)cc3)c3ccc4cccc5c6cccc7cccc(c3c45)c76)cc21 ; CC1(C)c2ccccc2-c2ccc(N(c3cc4c(cc3-c3cccc5c3N(c3ccccc3)COC5)-c3ccccc3C4(C)C)c3ccc4cccc5c6cccc7cccc(c3c45)c76)cc21 ; CC1(C)c2ccccc2C(C)(C)c2cc(N(c3ccc(-c4ccccc4)cc3)c3ccc(-c4ccccc4)cc3)c(-c3cccc4c3CB(c3ccccc3)O4)cc21]

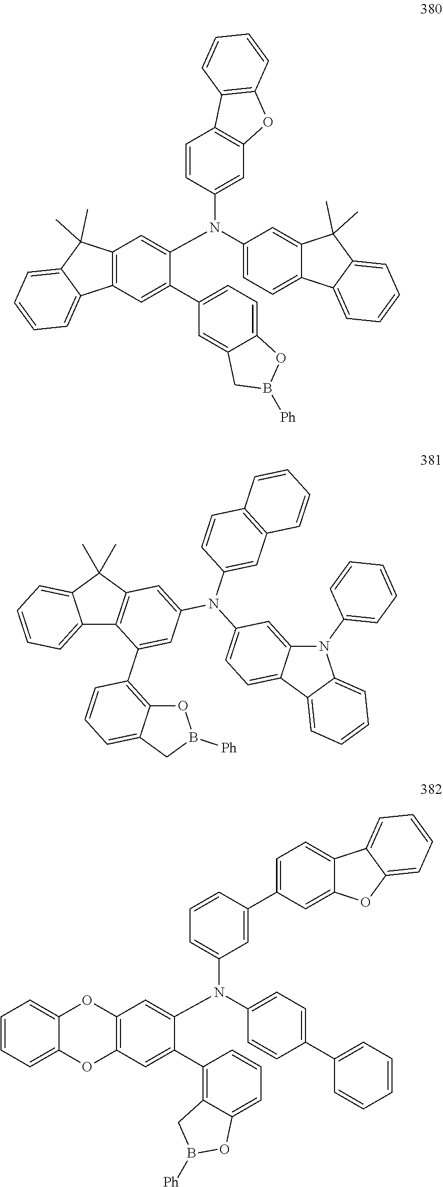 [SMILES: CC1(C)c2ccccc2-c2ccc(N(c3ccc4c(c3)oc3ccccc34)c3cc4c(cc3-c3ccc5c(c3)CB(c3ccccc3)O5)-c3ccccc3C4(C)C)cc21 ; CC1(C)c2ccccc2-c2c(-c3cccc4c3OB(c3ccccc3)C4)cc(N(c3ccc4ccccc4c3)c3ccc4c5ccccc5n(-c5ccccc5)c4c3)cc21 ; c1ccc(B2Cc3c(cccc3-c3cc4c(cc3N(c3ccc(-c5ccccc5)cc3)c3cccc(-c5ccc6c(c5)oc5ccccc56)c3)Oc3ccccc3O4)O2)cc1]

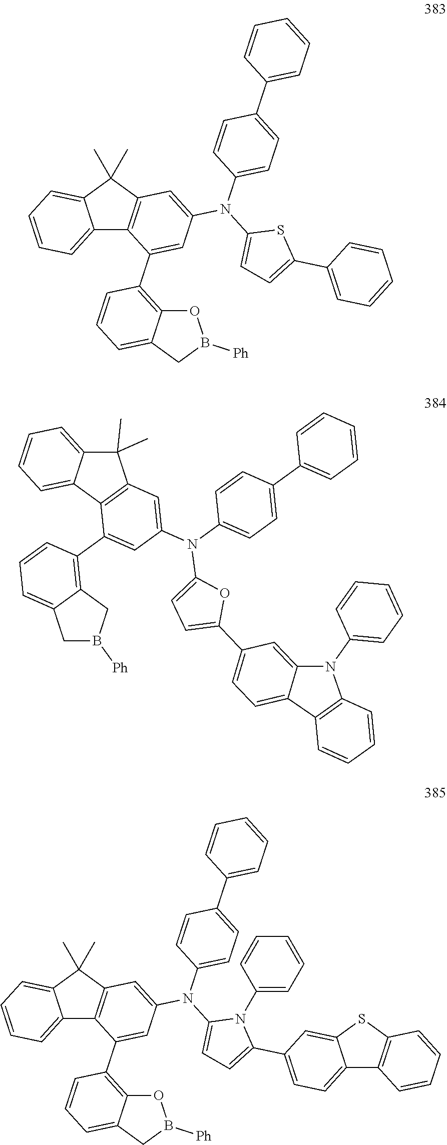 [SMILES: CC1(C)c2ccccc2-c2c(-c3cccc4c3OB(c3ccccc3)C4)cc(N(c3ccc(-c4ccccc4)cc3)c3ccc(-c4ccccc4)s3)cc21 ; CC1(C)c2ccccc2-c2c(-c3cccc4c3CB(c3ccccc3)C4)cc(N(c3ccc(-c4ccccc4)cc3)c3ccc(-c4ccc5c6ccccc6n(-c6ccccc6)c5c4)o3)cc21 ; CC1(C)c2ccccc2-c2c(-c3cccc4c3OB(c3ccccc3)C4)cc(N(c3ccc(-c4ccccc4)cc3)c3ccc(-c4ccc5c(c4)sc4ccccc45)n3-c3ccccc3)cc21]

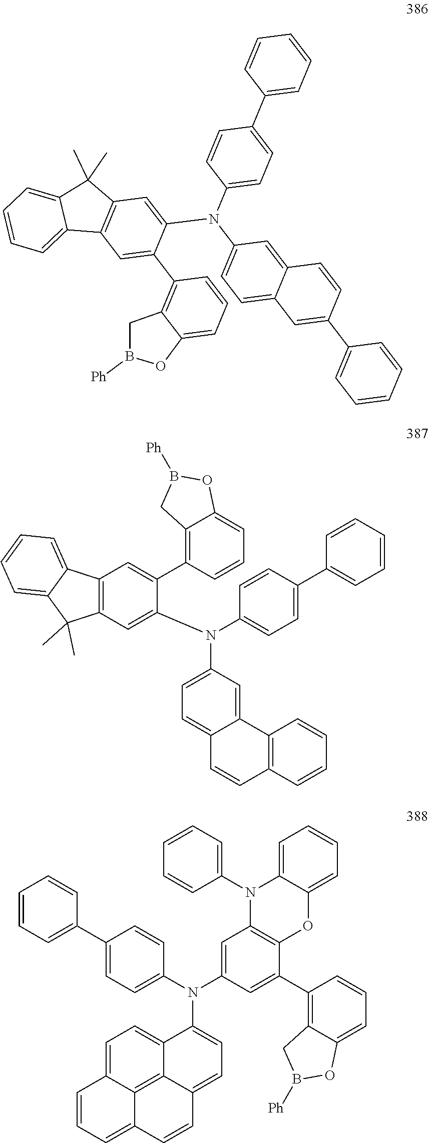

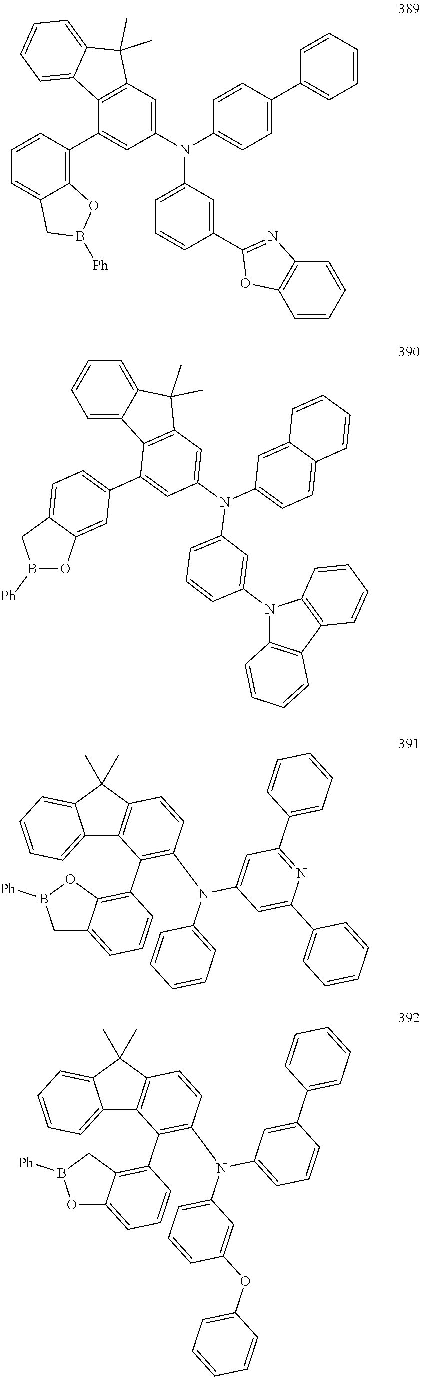

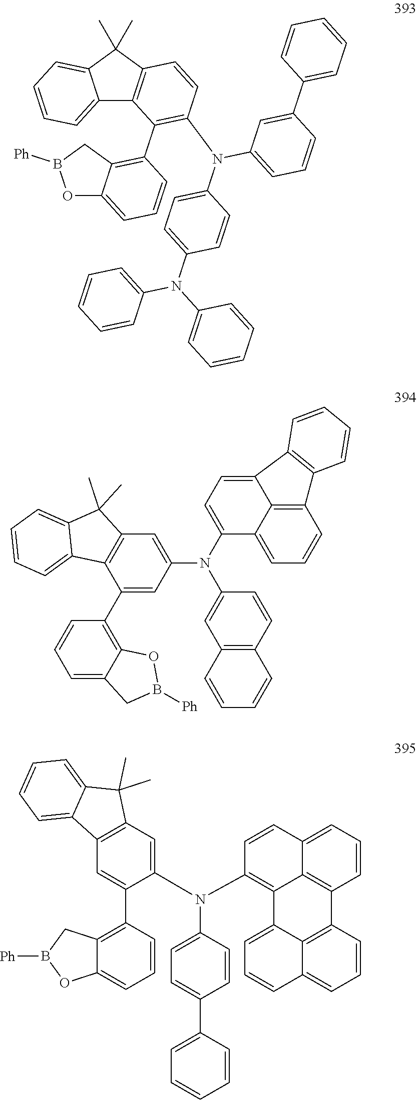 [SMILES: CC1(C)c2ccccc2-c2c1ccc(N(c1ccc(N(c3ccccc3)c3ccccc3)cc1)c1cccc(-c3ccccc3)c1)c2-c1cccc2c1CB(c1ccccc1)O2 ; CC1(C)c2ccccc2-c2c(-c3cccc4c3OB(c3ccccc3)C4)cc(N(c3ccc4ccccc4c3)c3ccc4c5c(cccc35)-c3ccccc3-4)cc21 ; CC1(C)c2ccccc2-c2cc(-c3cccc4c3CB(c3ccccc3)O4)c(N(c3ccc(-c4ccccc4)cc3)c3ccc4cccc5c6cccc7cccc(c3c45)c76)cc21]

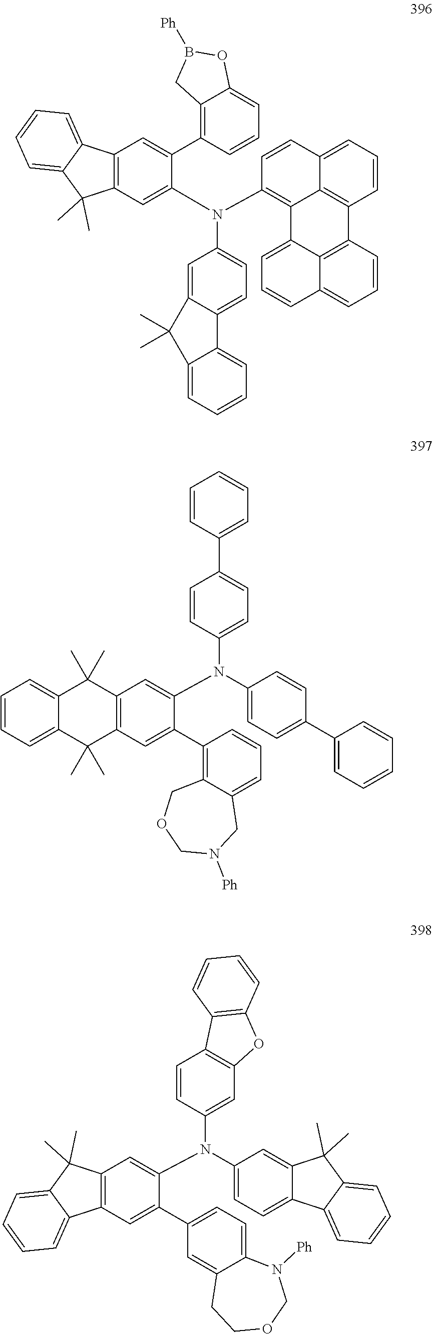 [SMILES: CC1(C)c2ccccc2-c2ccc(N(c3cc4c(cc3-c3cccc5c3CB(c3ccccc3)O5)-c3ccccc3C4(C)C)c3ccc4cccc5c6cccc7cccc(c3c45)c76)cc21 ; CC1(C)c2ccccc2C(C)(C)c2cc(N(c3ccc(-c4ccccc4)cc3)c3ccc(-c4ccccc4)cc3)c(-c3cccc4c3COCN(c3ccccc3)C4)cc21 ; CC1(C)c2ccccc2-c2ccc(N(c3ccc4c(c3)oc3ccccc34)c3cc4c(cc3-c3ccc5c(c3)CCOCN5c3ccccc3)-c3ccccc3C4(C)C)cc21]

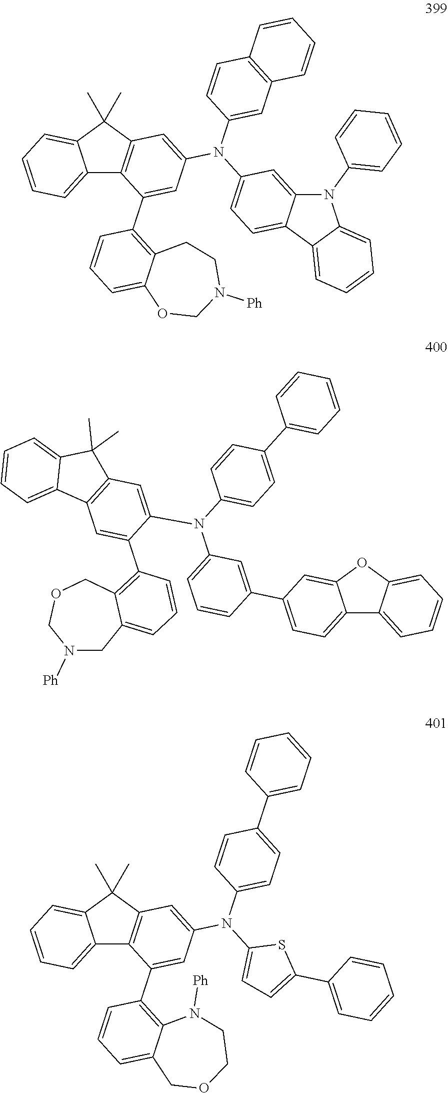 [SMILES: CC1(C)c2ccccc2-c2c(-c3cccc4c3CCN(c3ccccc3)CO4)cc(N(c3ccc4ccccc4c3)c3ccc4c5ccccc5n(-c5ccccc5)c4c3)cc21 ; CC1(C)c2ccccc2-c2cc(-c3cccc4c3COCN(c3ccccc3)C4)c(N(c3ccc(-c4ccccc4)cc3)c3cccc(-c4ccc5c(c4)oc4ccccc45)c3)cc21 ; CC1(C)c2ccccc2-c2c(-c3cccc4c3N(c3ccccc3)CCOC4)cc(N(c3ccc(-c4ccccc4)cc3)c3ccc(-c4ccccc4)s3)cc21]

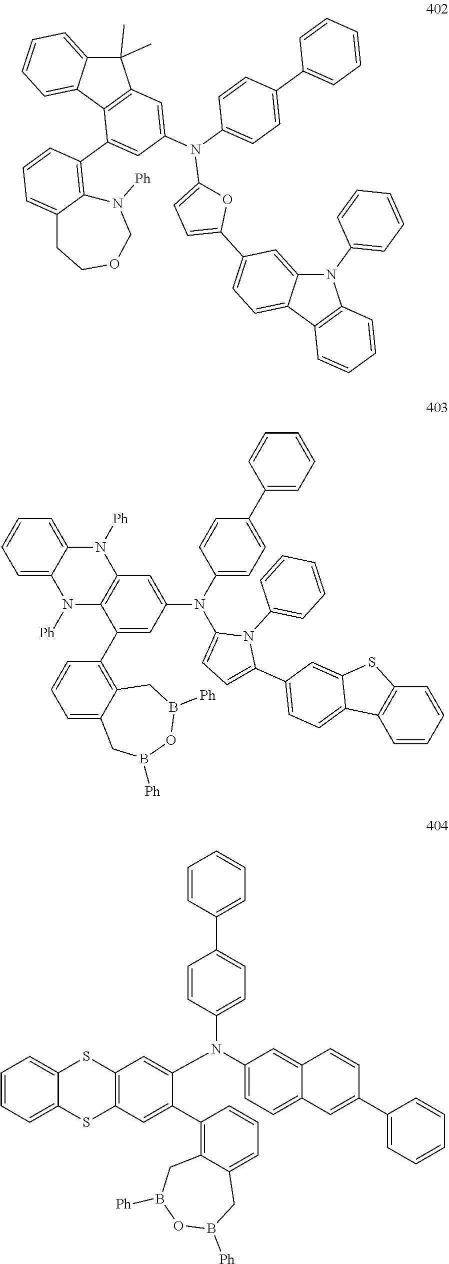 [SMILES: CC1(C)c2ccccc2-c2c(-c3cccc4c3N(c3ccccc3)COCC4)cc(N(c3ccc(-c4ccccc4)cc3)c3ccc(-c4ccc5c6ccccc6n(-c6ccccc6)c5c4)o3)cc21 ; c1ccc(B2Cc3cccc(-c4cc(N(c5ccc(-c6ccccc6)cc5)c5ccc(-c6ccc7c(c6)sc6ccccc67)n5-c5ccccc5)cc5c4N(c4ccccc4)c4ccccc4N5c4ccccc4)c3CB(c3ccccc3)O2)cc1 ; c1ccc(B2Cc3cccc(-c4cc5c(cc4N(c4ccc(-c6ccccc6)cc4)c4ccc6cc(-c7ccccc7)ccc6c4)Sc4ccccc4S5)c3CB(c3ccccc3)O2)cc1]

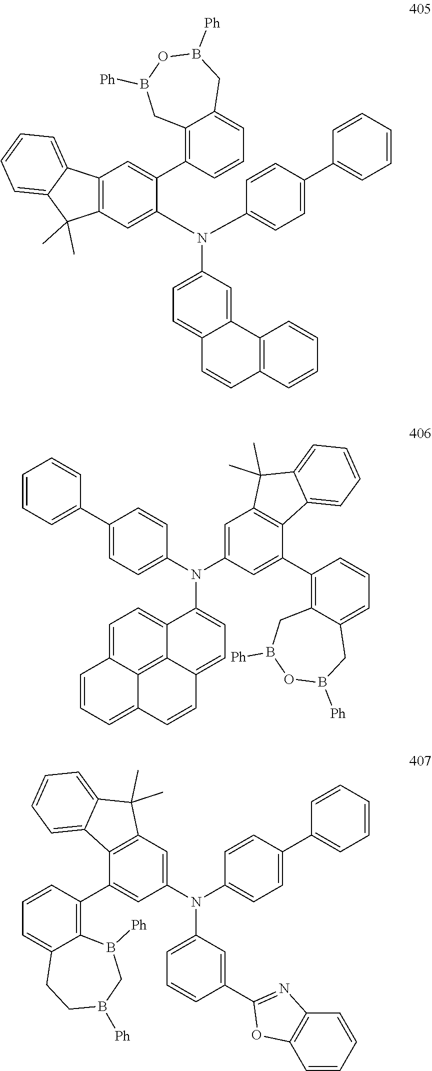 [SMILES: CC1(C)c2ccccc2-c2cc(-c3cccc4c3CB(c3ccccc3)OB(c3ccccc3)C4)c(N(c3ccc(-c4ccccc4)cc3)c3ccc4ccc5ccccc5c4c3)cc21 ; CC1(C)c2ccccc2-c2c(-c3cccc4c3CB(c3ccccc3)OB(c3ccccc3)C4)cc(N(c3ccc(-c4ccccc4)cc3)c3ccc4ccc5cccc6ccc3c4c56)cc21 ; CC1(C)c2ccccc2-c2c(-c3cccc4c3B(c3ccccc3)CB(c3ccccc3)CC4)cc(N(c3ccc(-c4ccccc4)cc3)c3cccc(-c4nc5ccccc5o4)c3)cc21]

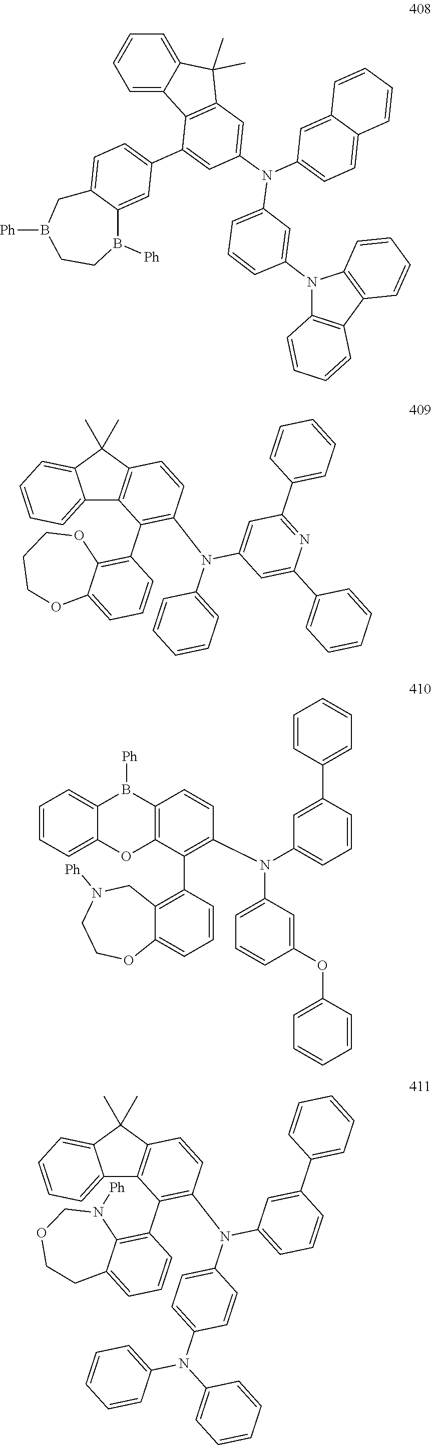

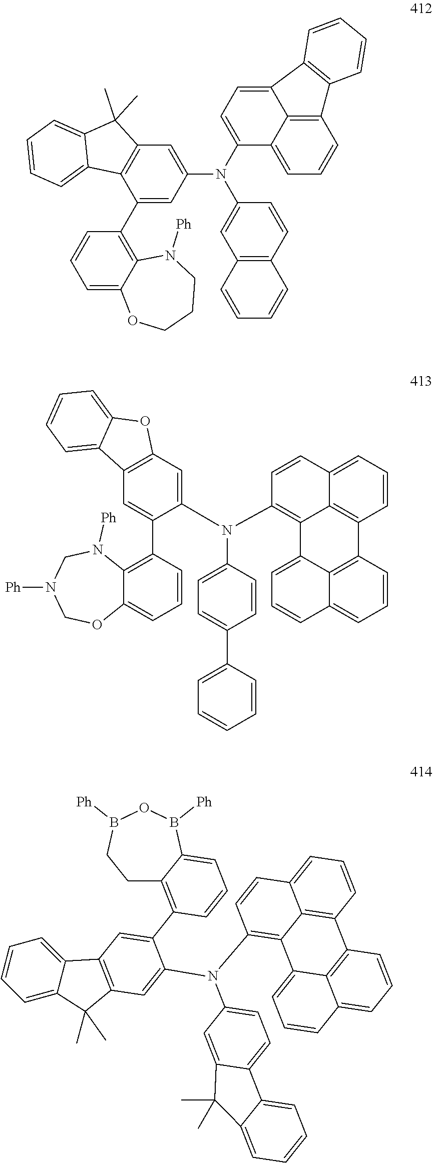 [SMILES: CC1(C)c2ccccc2-c2c(-c3cccc4c3N(c3ccccc3)CCCO4)cc(N(c3ccc4ccccc4c3)c3ccc4c5c(cccc35)-c3ccccc3-4)cc21 ; c1ccc(-c2ccc(N(c3cc4oc5ccccc5c4cc3-c3cccc4c3N(c3ccccc3)CN(c3ccccc3)CO4)c3ccc4cccc5c6cccc7cccc(c3c45)c76)cc2)cc1 ; CC1(C)c2ccccc2-c2ccc(N(c3cc4c(cc3-c3cccc5c3CCB(c3ccccc3)OB5c3ccccc3)-c3ccccc3C4(C)C)c3ccc4cccc5c6cccc7cccc(c3c45)c76)cc21]

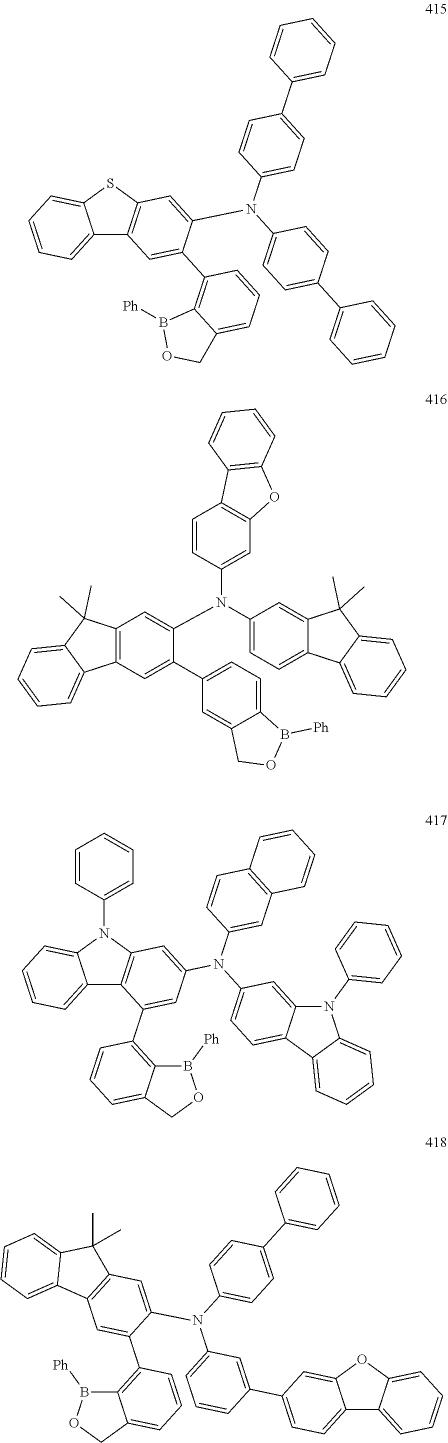 [SMILES: c1ccc(B2OCc3cccc(-c4cc5c(cc4N(c4ccc(-c6ccccc6)cc4)c4ccc(-c6ccccc6)cc4)sc4ccccc45)c32)cc1 ; CC1(C)c2ccccc2-c2ccc(N(c3ccc4c(c3)oc3ccccc34)c3cc4c(cc3-c3ccc5c(c3)COB5c3ccccc3)-c3ccccc3C4(C)C)cc21 ; c1ccc(B2OCc3cccc(-c4cc(N(c5ccc6ccccc6c5)c5ccc6c7ccccc7n(-c7ccccc7)c6c5)cc5c4c4ccccc4n5-c4ccccc4)c32)cc1 ; CC1(C)c2ccccc2-c2cc(-c3cccc4c3B(c3ccccc3)OC4)c(N(c3ccc(-c4ccccc4)cc3)c3cccc(-c4ccc5c(c4)oc4ccccc45)c3)cc21]

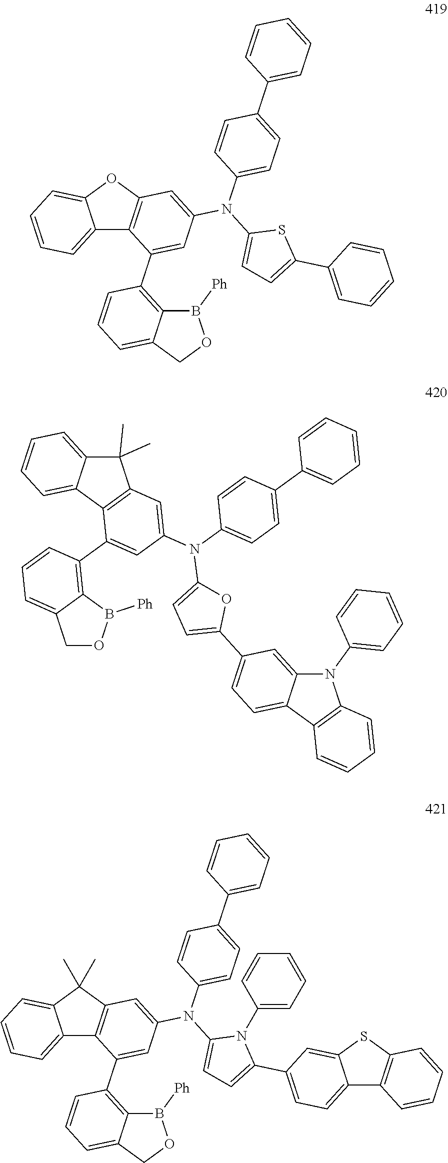 [SMILES: c1ccc(B2OCc3cccc(-c4cc(N(c5ccc(-c6ccccc6)cc5)c5ccc(-c6ccccc6)s5)cc5oc6ccccc6c45)c32)cc1 ; CC1(C)c2ccccc2-c2c(-c3cccc4c3B(c3ccccc3)OC4)cc(N(c3ccc(-c4ccccc4)cc3)c3ccc(-c4ccc5c6ccccc6n(-c6ccccc6)c5c4)o3)cc21 ; CC1(C)c2ccccc2-c2c(-c3cccc4c3B(c3ccccc3)OC4)cc(N(c3ccc(-c4ccccc4)cc3)c3ccc(-c4ccc5c(c4)sc4ccccc45)n3-c3ccccc3)cc21]

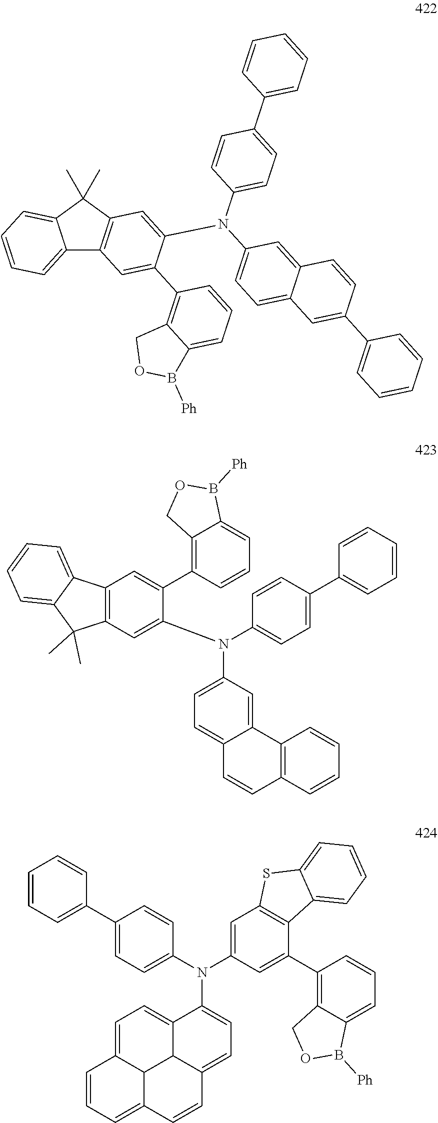 [SMILES: CC1(C)c2ccccc2-c2cc(-c3cccc4c3COB4c3ccccc3)c(N(c3ccc(-c4ccccc4)cc3)c3ccc4cc(-c5ccccc5)ccc4c3)cc21 ; CC1(C)c2ccccc2-c2cc(-c3cccc4c3COB4c3ccccc3)c(N(c3ccc(-c4ccccc4)cc3)c3ccc4ccc5ccccc5c4c3)cc21 ; C1=CC2=CC=C3C(N(c4ccc(-c5ccccc5)cc4)c4cc(-c5cccc6c5COB6c5ccccc5)c5c(c4)sc4ccccc45)=CC=C4C=CC(=C1)C2C43]

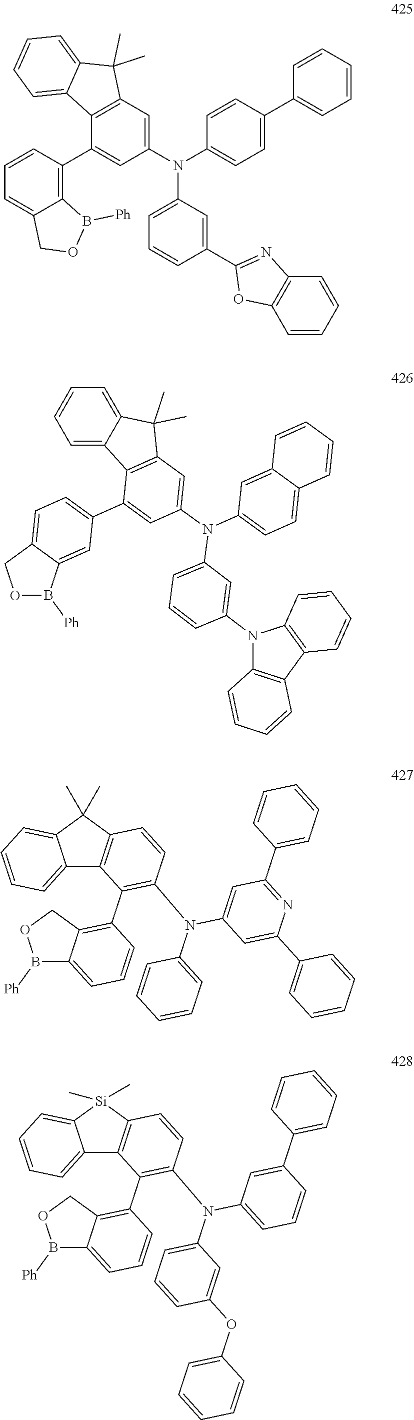 [SMILES: CC1(C)c2ccccc2-c2c(-c3cccc4c3B(c3ccccc3)OC4)cc(N(c3ccc(-c4ccccc4)cc3)c3cccc(-c4nc5ccccc5o4)c3)cc21 ; CC1(C)c2ccccc2-c2c(-c3ccc4c(c3)B(c3ccccc3)OC4)cc(N(c3cccc(-n4c5ccccc5c5ccccc54)c3)c3ccc4ccccc4c3)cc21 ; CC1(C)c2ccccc2-c2c1ccc(N(c1ccccc1)c1cc(-c3ccccc3)nc(-c3ccccc3)c1)c2-c1cccc2c1COB2c1ccccc1 ; C[Si]1(C)c2ccccc2-c2c1ccc(N(c1cccc(Oc3ccccc3)c1)c1cccc(-c3ccccc3)c1)c2-c1cccc2c1COB2c1ccccc1]

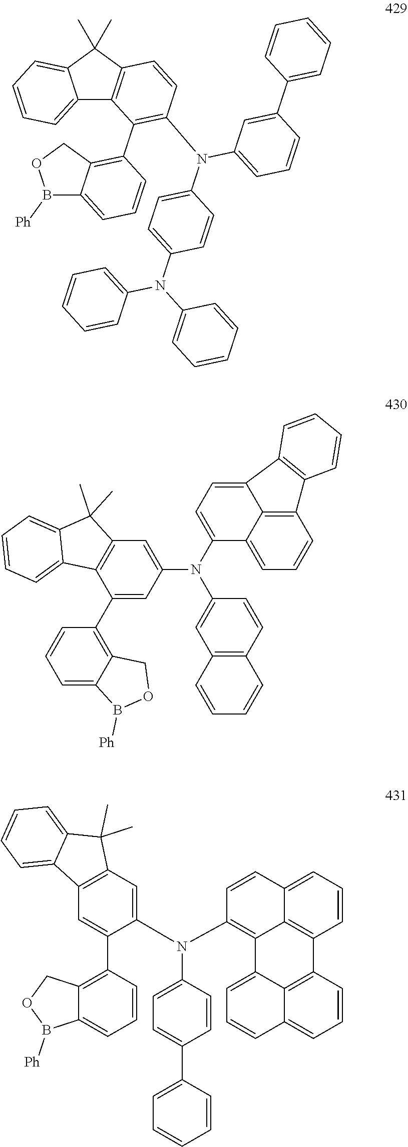 [SMILES: CC1(C)c2ccccc2-c2c1ccc(N(c1ccc(N(c3ccccc3)c3ccccc3)cc1)c1cccc(-c3ccccc3)c1)c2-c1cccc2c1COB2c1ccccc1 ; CC1(C)c2ccccc2-c2c(-c3cccc4c3COB4c3ccccc3)cc(N(c3ccc4ccccc4c3)c3ccc4c5c(cccc35)-c3ccccc3-4)cc21 ; CC1(C)c2ccccc2-c2cc(-c3cccc4c3COB4c3ccccc3)c(N(c3ccc(-c4ccccc4)cc3)c3ccc4cccc5c6cccc7cccc(c3c45)c76)cc21]

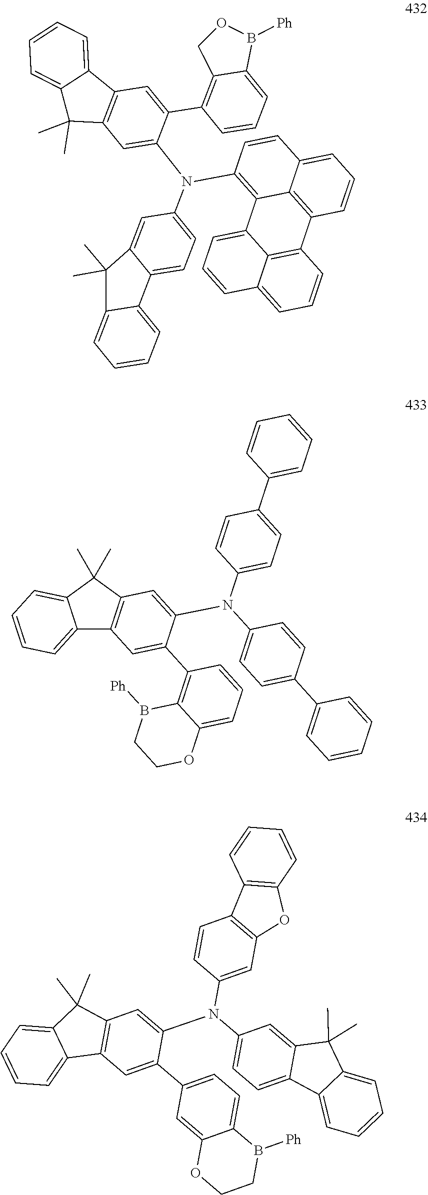 [SMILES: CC1(C)c2ccccc2-c2ccc(N(c3cc4c(cc3-c3cccc5c3COB5c3ccccc3)-c3ccccc3C4(C)C)c3ccc4cccc5c6cccc7cccc(c3c45)c76)cc21 ; CC1(C)c2ccccc2-c2cc(-c3cccc4c3B(c3ccccc3)CCO4)c(N(c3ccc(-c4ccccc4)cc3)c3ccc(-c4ccccc4)cc3)cc21 ; CC1(C)c2ccccc2-c2ccc(N(c3ccc4c(c3)oc3ccccc34)c3cc4c(cc3-c3ccc5c(c3)OCCB5c3ccccc3)-c3ccccc3C4(C)C)cc21]

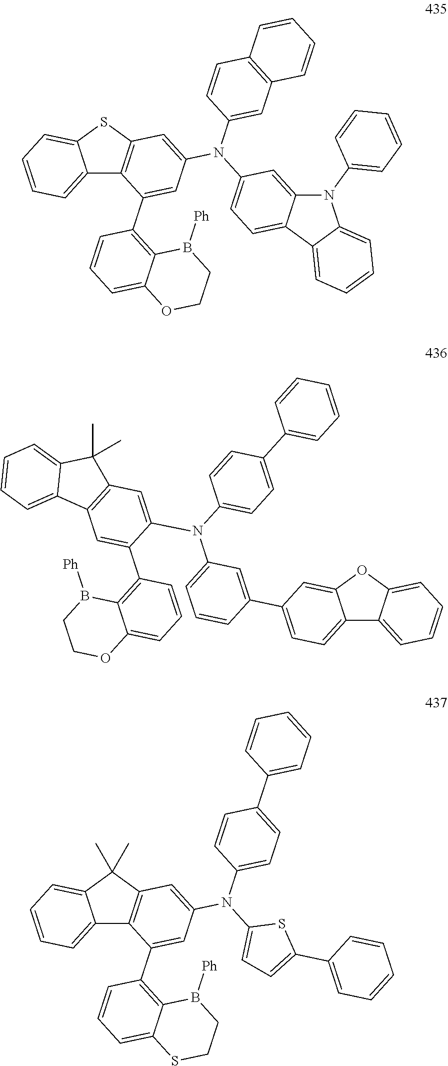 [SMILES: c1ccc(B2CCOc3cccc(-c4cc(N(c5ccc6ccccc6c5)c5ccc6c7ccccc7n(-c7ccccc7)c6c5)cc5sc6ccccc6c45)c32)cc1 ; CC1(C)c2ccccc2-c2cc(-c3cccc4c3B(c3ccccc3)CCO4)c(N(c3ccc(-c4ccccc4)cc3)c3cccc(-c4ccc5c(c4)oc4ccccc45)c3)cc21 ; CC1(C)c2ccccc2-c2c(-c3cccc4c3B(c3ccccc3)CCS4)cc(N(c3ccc(-c4ccccc4)cc3)c3ccc(-c4ccccc4)s3)cc21]

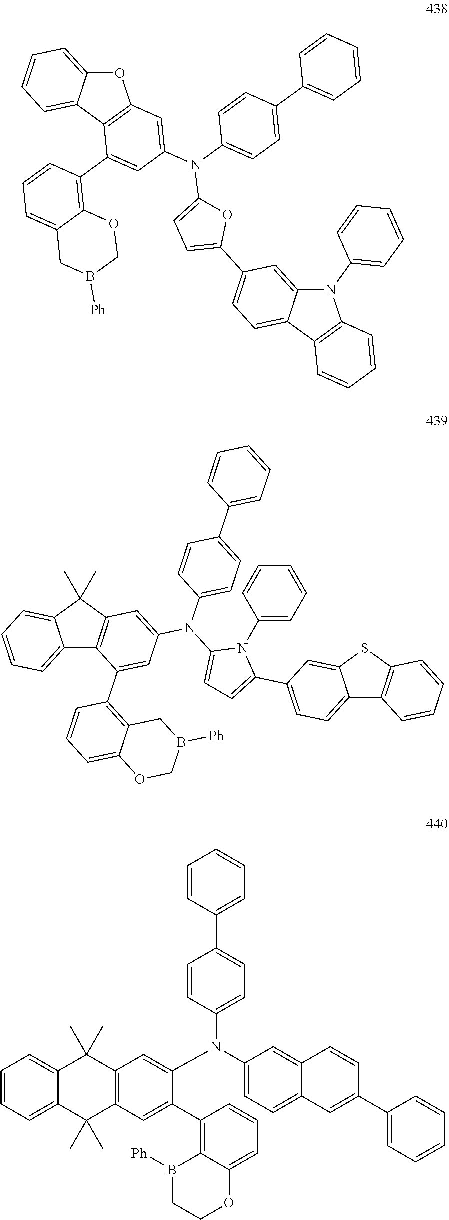 [SMILES: c1ccc(B2COc3c(cccc3-c3cc(N(c4ccc(-c5ccccc5)cc4)c4ccc(-c5ccc6c7ccccc7n(-c7ccccc7)c6c5)o4)cc4oc5ccccc5c34)C2)cc1 ; CC1(C)c2ccccc2-c2c(-c3cccc4c3CB(c3ccccc3)CO4)cc(N(c3ccc(-c4ccccc4)cc3)c3ccc(-c4ccc5c(c4)sc4ccccc45)n3-c3ccccc3)cc21 ; CC1(C)c2ccccc2C(C)(C)c2cc(N(c3ccc(-c4ccccc4)cc3)c3ccc4cc(-c5ccccc5)ccc4c3)c(-c3cccc4c3B(c3ccccc3)CCO4)cc21]

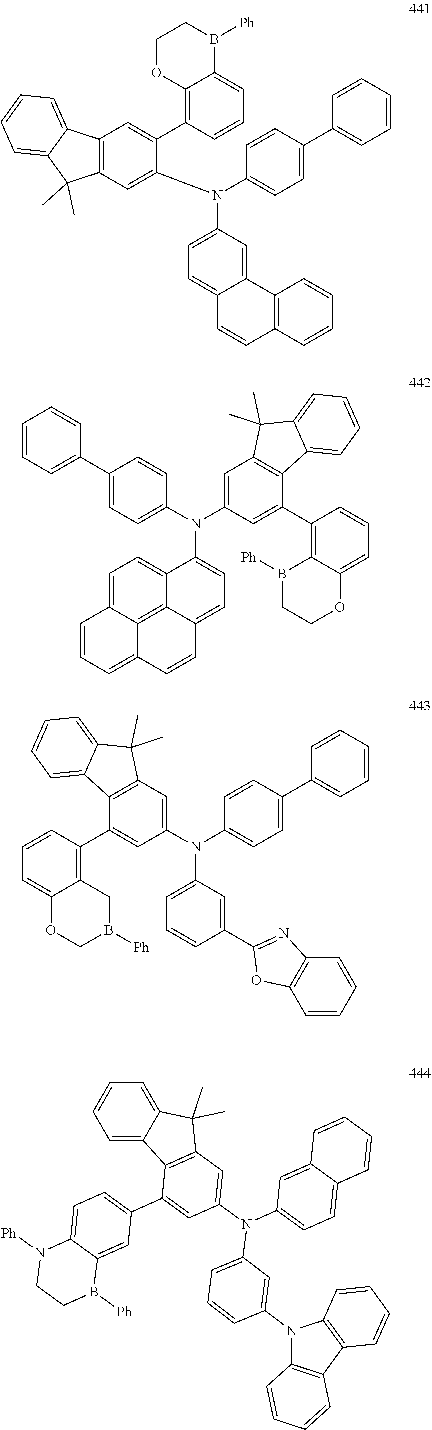 [SMILES: CC1(C)c2ccccc2-c2cc(-c3cccc4c3OCCB4c3ccccc3)c(N(c3ccc(-c4ccccc4)cc3)c3ccc4ccc5ccccc5c4c3)cc21 ; CC1(C)c2ccccc2-c2c(-c3cccc4c3B(c3ccccc3)CCO4)cc(N(c3ccc(-c4ccccc4)cc3)c3ccc4ccc5cccc6ccc3c4c56)cc21 ; CC1(C)c2ccccc2-c2c(-c3cccc4c3CB(c3ccccc3)CO4)cc(N(c3ccc(-c4ccccc4)cc3)c3cccc(-c4nc5ccccc5o4)c3)cc21 ; CC1(C)c2ccccc2-c2c(-c3ccc4c(c3)B(c3ccccc3)CCN4c3ccccc3)cc(N(c3cccc(-n4c5ccccc5c5ccccc54)c3)c3ccc4ccccc4c3)cc21]

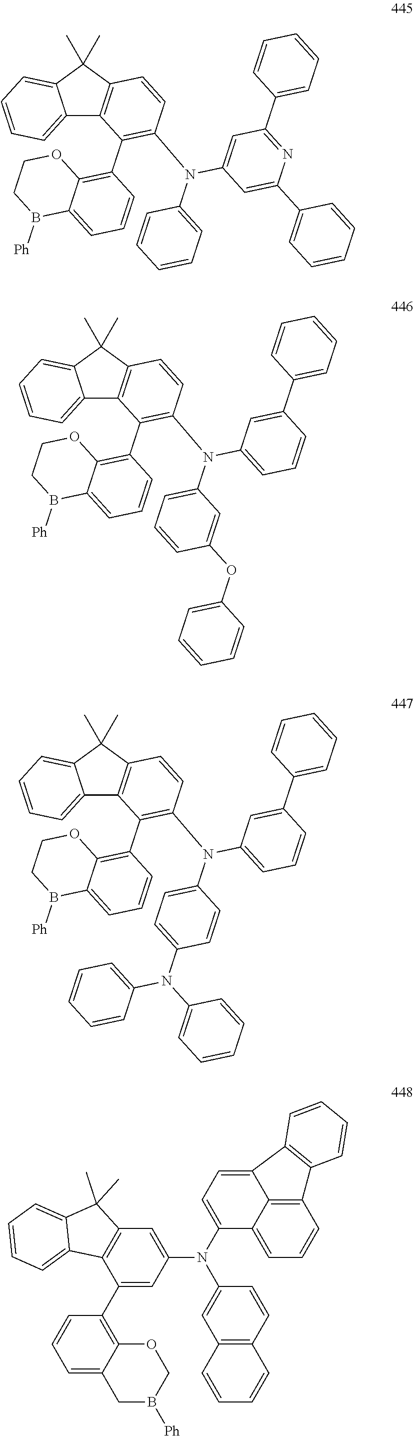 [SMILES: CC1(C)c2ccccc2-c2c1ccc(N(c1ccccc1)c1cc(-c3ccccc3)nc(-c3ccccc3)c1)c2-c1cccc2c1OCCB2c1ccccc1 ; CC1(C)c2ccccc2-c2c1ccc(N(c1cccc(Oc3ccccc3)c1)c1cccc(-c3ccccc3)c1)c2-c1cccc2c1OCCB2c1ccccc1 ; CC1(C)c2ccccc2-c2c1ccc(N(c1ccc(N(c3ccccc3)c3ccccc3)cc1)c1cccc(-c3ccccc3)c1)c2-c1cccc2c1OCCB2c1ccccc1 ; CC1(C)c2ccccc2-c2c(-c3cccc4c3OCB(c3ccccc3)C4)cc(N(c3ccc4ccccc4c3)c3ccc4c5c(cccc35)-c3ccccc3-4)cc21]

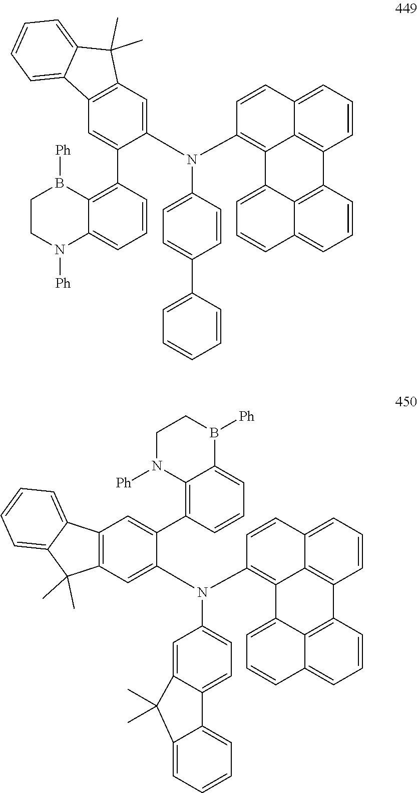 [SMILES: CC1(C)c2ccccc2-c2cc(-c3cccc4c3B(c3ccccc3)CCN4c3ccccc3)c(N(c3ccc(-c4ccccc4)cc3)c3ccc4cccc5c6cccc7cccc(c3c45)c76)cc21 ; CC1(C)c2ccccc2-c2ccc(N(c3cc4c(cc3-c3cccc5c3N(c3ccccc3)CCB5c3ccccc3)-c3ccccc3C4(C)C)c3ccc4cccc5c6cccc7cccc(c3c45)c76)cc21]

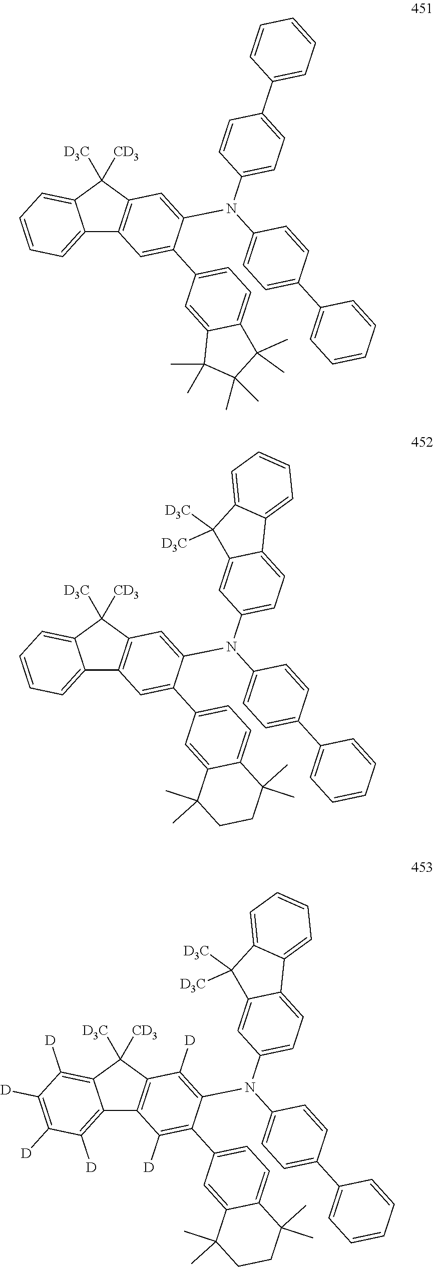

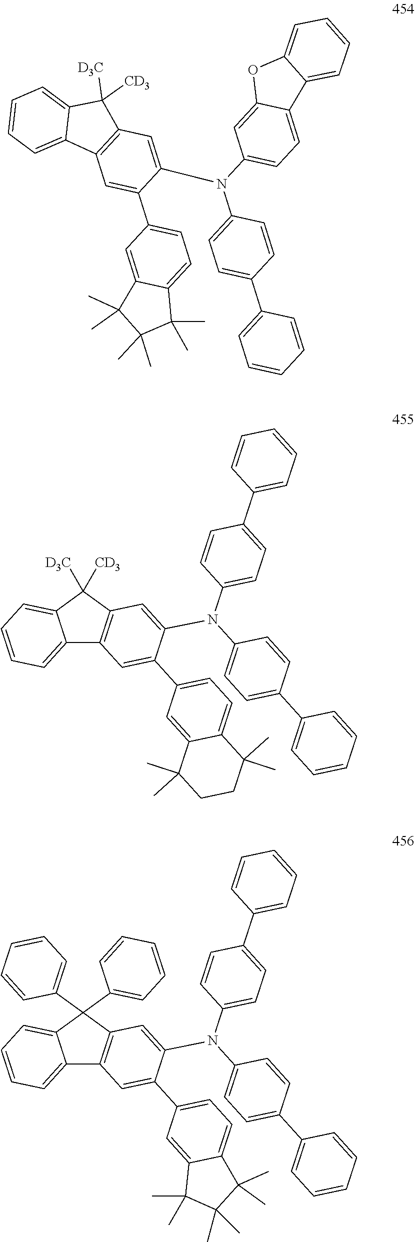 [SMILES: [2H]C([2H])([2H])C1(C([2H])([2H])[2H])c2ccccc2-c2cc(-c3ccc4c(c3)C(C)(C)C(C)(C)C4(C)C)c(N(c3ccc(-c4ccccc4)cc3)c3ccc4c(c3)oc3ccccc34)cc21 ; [2H]C([2H])([2H])C1(C([2H])([2H])[2H])c2ccccc2-c2cc(-c3ccc4c(c3)C(C)(C)CCC4(C)C)c(N(c3ccc(-c4ccccc4)cc3)c3ccc(-c4ccccc4)cc3)cc21 ; CC1(C)c2ccc(-c3cc4c(cc3N(c3ccc(-c5ccccc5)cc3)c3ccc(-c5ccccc5)cc3)C(c3ccccc3)(c3ccccc3)c3ccccc3-4)cc2C(C)(C)C1(C)C]

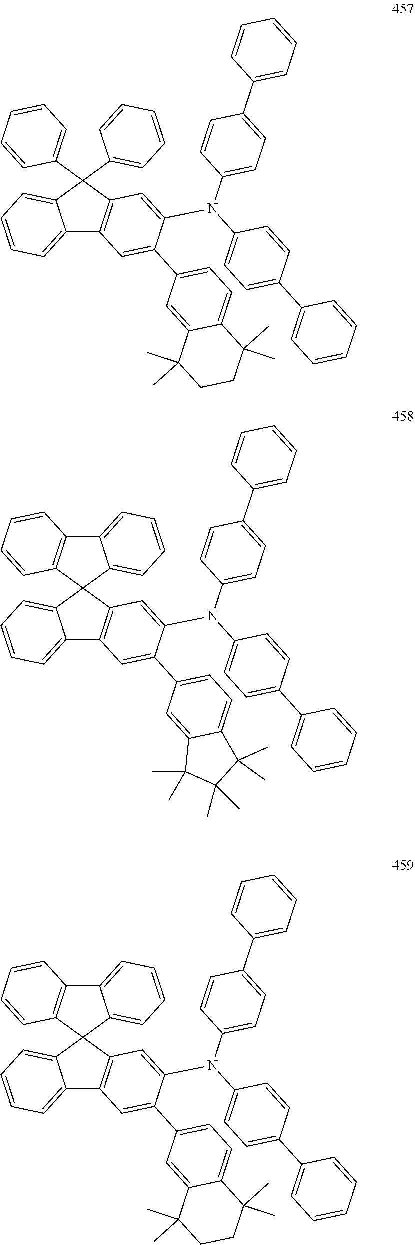 [SMILES: CC1(C)CCC(C)(C)c2cc(-c3cc4c(cc3N(c3ccc(-c5ccccc5)cc3)c3ccc(-c5ccccc5)cc3)C(c3ccccc3)(c3ccccc3)c3ccccc3-4)ccc21 ; CC1(C)c2ccc(-c3cc4c(cc3N(c3ccc(-c5ccccc5)cc3)c3ccc(-c5ccccc5)cc3)C3(c5ccccc5-c5ccccc53)c3ccccc3-4)cc2C(C)(C)C1(C)C ; CC1(C)CCC(C)(C)c2cc(-c3cc4c(cc3N(c3ccc(-c5ccccc5)cc3)c3ccc(-c5ccccc5)cc3)C3(c5ccccc5-c5ccccc53)c3ccccc3-4)ccc21]

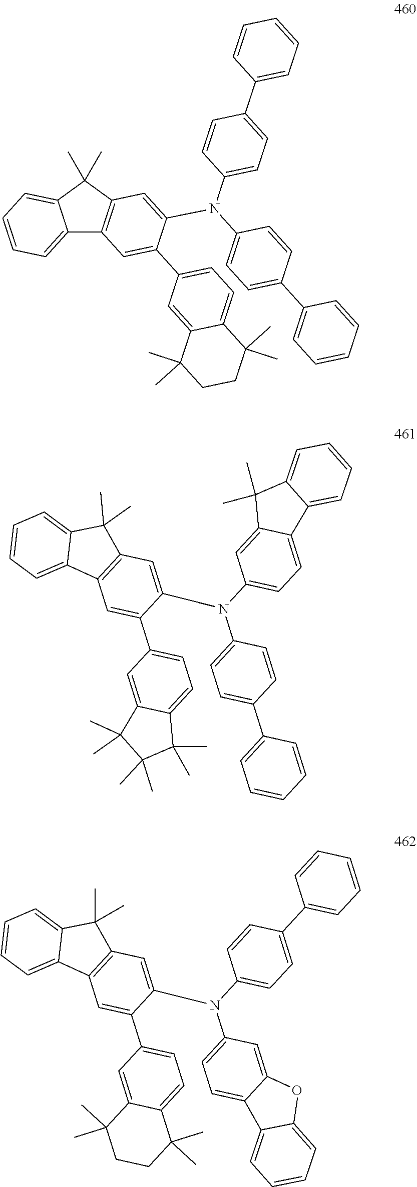 [SMILES: CC1(C)CCC(C)(C)c2cc(-c3cc4c(cc3N(c3ccc(-c5ccccc5)cc3)c3ccc(-c5ccccc5)cc3)C(C)(C)c3ccccc3-4)ccc21 ; CC1(C)c2ccccc2-c2ccc(N(c3ccc(-c4ccccc4)cc3)c3cc4c(cc3-c3ccc5c(c3)C(C)(C)C(C)(C)C5(C)C)-c3ccccc3C4(C)C)cc21 ; CC1(C)CCC(C)(C)c2cc(-c3cc4c(cc3N(c3ccc(-c5ccccc5)cc3)c3ccc5c(c3)oc3ccccc35)C(C)(C)c3ccccc3-4)ccc21]

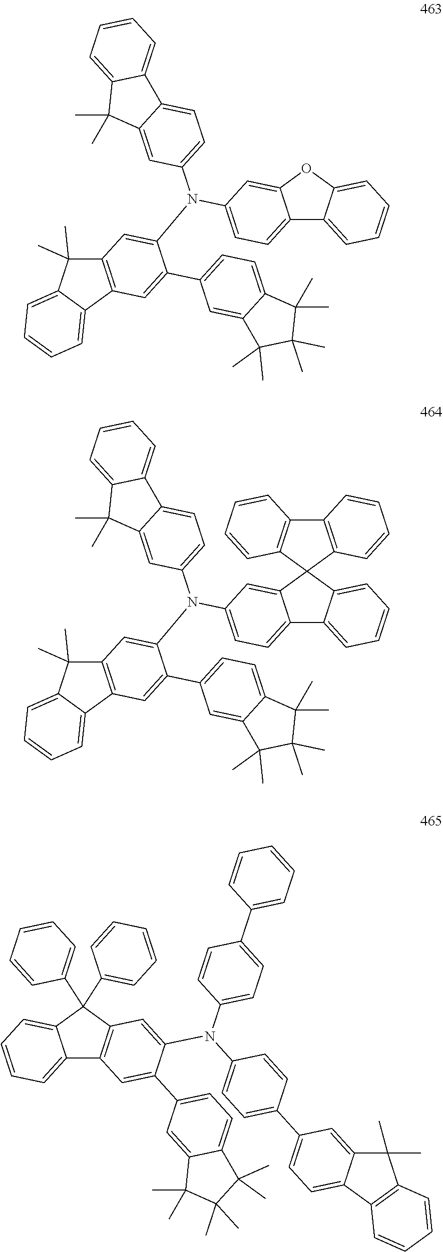 [SMILES: CC1(C)c2ccccc2-c2ccc(N(c3ccc4c(c3)oc3ccccc34)c3cc4c(cc3-c3ccc5c(c3)C(C)(C)C(C)(C)C5(C)C)-c3ccccc3C4(C)C)cc21 ; CC1(C)c2ccccc2-c2ccc(N(c3ccc4c(c3)C3(c5ccccc5-c5ccccc53)c3ccccc3-4)c3cc4c(cc3-c3ccc5c(c3)C(C)(C)C(C)(C)C5(C)C)-c3ccccc3C4(C)C)cc21 ; CC1(C)c2ccccc2-c2ccc(-c3ccc(N(c4ccc(-c5ccccc5)cc4)c4cc5c(cc4-c4ccc6c(c4)C(C)(C)C(C)(C)C6(C)C)-c4ccccc4C5(c4ccccc4)c4ccccc4)cc3)cc21]

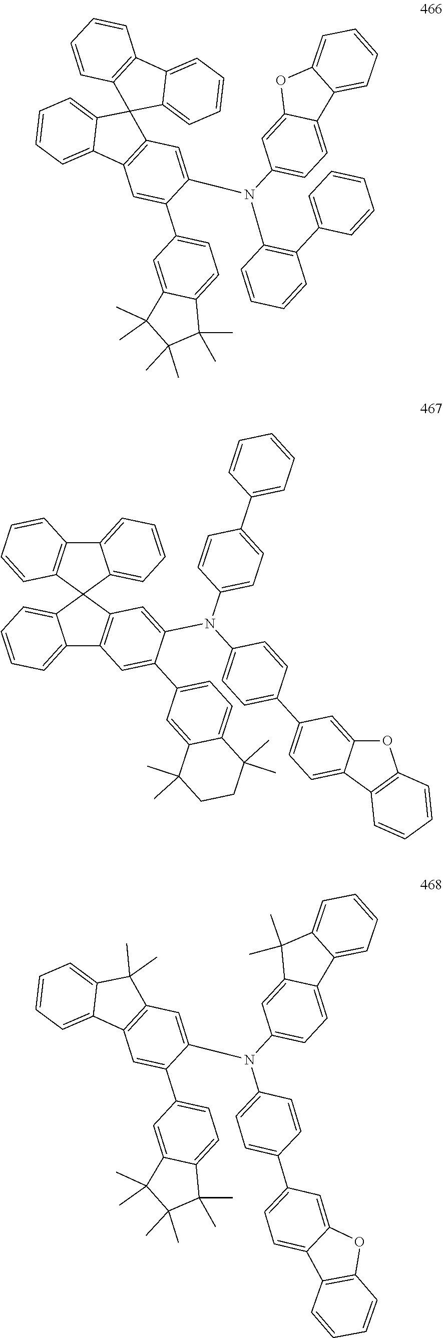 [SMILES: CC1(C)c2ccc(-c3cc4c(cc3N(c3ccc5c(c3)oc3ccccc35)c3ccccc3-c3ccccc3)C3(c5ccccc5-c5ccccc53)c3ccccc3-4)cc2C(C)(C)C1(C)C ; CC1(C)CCC(C)(C)c2cc(-c3cc4c(cc3N(c3ccc(-c5ccccc5)cc3)c3ccc(-c5ccc6c(c5)oc5ccccc56)cc3)C3(c5ccccc5-c5ccccc53)c3ccccc3-4)ccc21 ; CC1(C)c2ccccc2-c2ccc(N(c3ccc(-c4ccc5c(c4)oc4ccccc45)cc3)c3cc4c(cc3-c3ccc5c(c3)C(C)(C)C(C)(C)C5(C)C)-c3ccccc3C4(C)C)cc21]

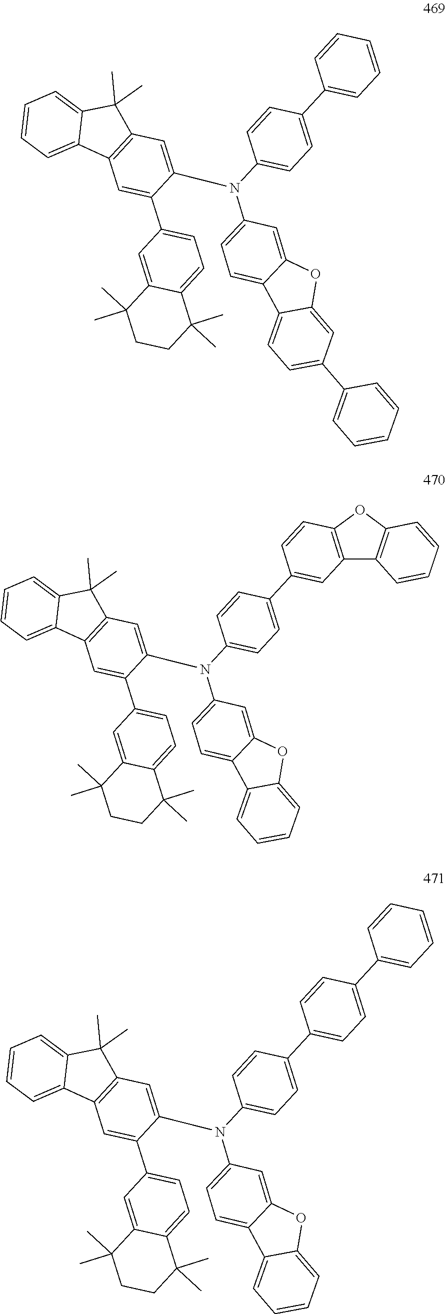 [SMILES: CC1(C)CCC(C)(C)c2cc(-c3cc4c(cc3N(c3ccc(-c5ccccc5)cc3)c3ccc5c(c3)oc3cc(-c6ccccc6)ccc35)C(C)(C)c3ccccc3-4)ccc21 ; CC1(C)CCC(C)(C)c2cc(-c3cc4c(cc3N(c3ccc(-c5ccc6oc7ccccc7c6c5)cc3)c3ccc5c(c3)oc3ccccc35)C(C)(C)c3ccccc3-4)ccc21 ; CC1(C)CCC(C)(C)c2cc(-c3cc4c(cc3N(c3ccc(-c5ccc(-c6ccccc6)cc5)cc3)c3ccc5c(c3)oc3ccccc35)C(C)(C)c3ccccc3-4)ccc21]

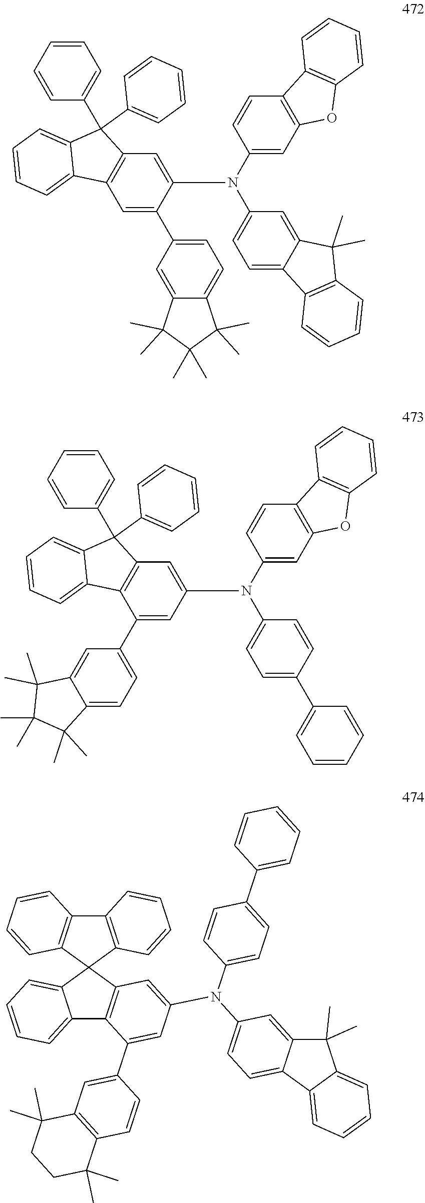

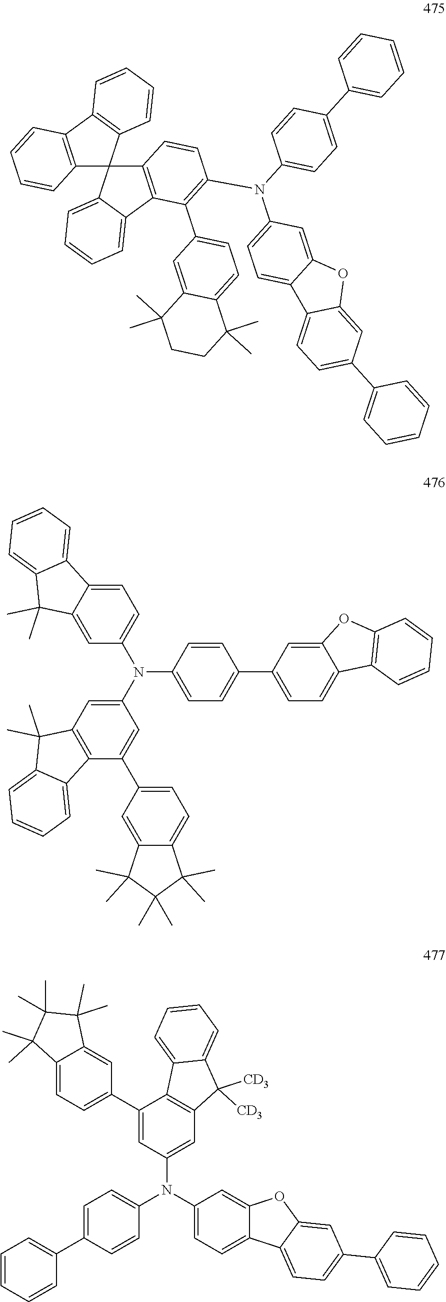 [SMILES: CC1(C)CCC(C)(C)c2cc(-c3c(N(c4ccc(-c5ccccc5)cc4)c4ccc5c(c4)oc4cc(-c6ccccc6)ccc45)ccc4c3-c3ccccc3C43c4ccccc4-c4ccccc43)ccc21 ; CC1(C)c2ccccc2-c2ccc(N(c3ccc(-c4ccc5c(c4)oc4ccccc45)cc3)c3cc(-c4ccc5c(c4)C(C)(C)C(C)(C)C5(C)C)c4c(c3)C(C)(C)c3ccccc3-4)cc21 ; [2H]C([2H])([2H])C1(C([2H])([2H])[2H])c2ccccc2-c2c(-c3ccc4c(c3)C(C)(C)C(C)(C)C4(C)C)cc(N(c3ccc(-c4ccccc4)cc3)c3ccc4c(c3)oc3cc(-c5ccccc5)ccc34)cc21]

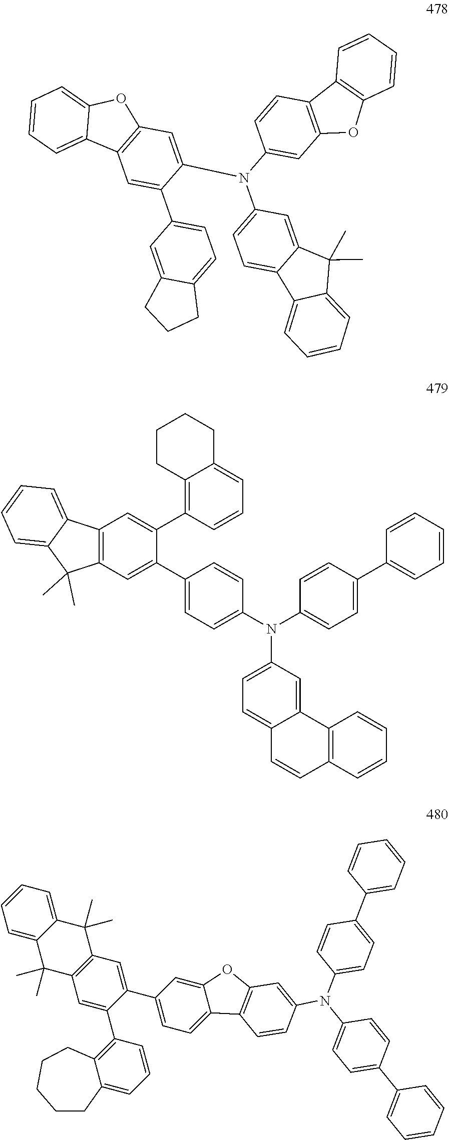 [SMILES: CC1(C)c2ccccc2-c2ccc(N(c3ccc4c(c3)oc3ccccc34)c3cc4oc5ccccc5c4cc3-c3ccc4c(c3)CCC4)cc21 ; CC1(C)c2ccccc2-c2cc(-c3cccc4c3CCCC4)c(-c3ccc(N(c4ccc(-c5ccccc5)cc4)c4ccc5ccc6ccccc6c5c4)cc3)cc21 ; CC1(C)c2ccccc2C(C)(C)c2cc(-c3cccc4c3CCCCC4)c(-c3ccc4c(c3)oc3cc(N(c5ccc(-c6ccccc6)cc5)c5ccc(-c6ccccc6)cc5)ccc34)cc21]

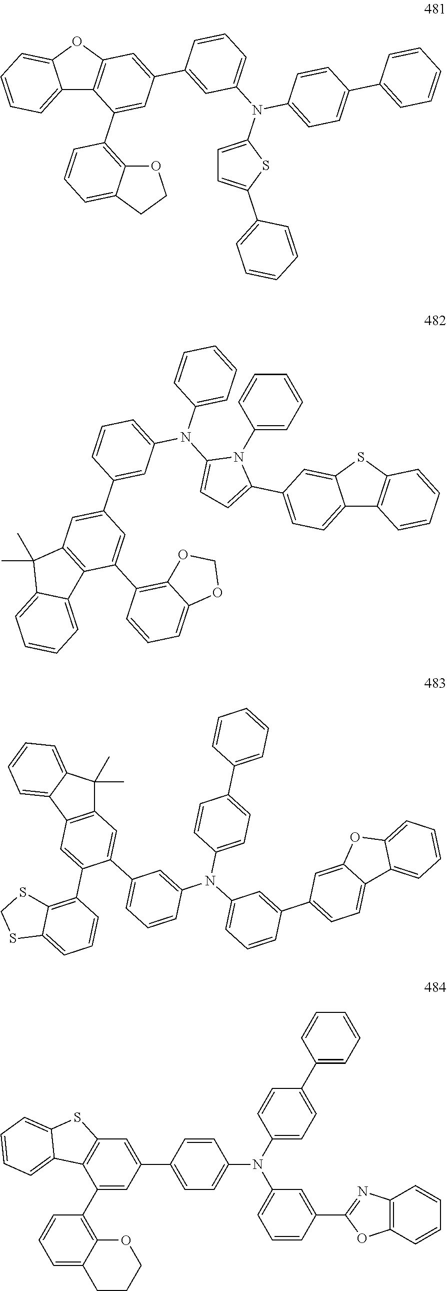 [SMILES: c1ccc(-c2ccc(N(c3cccc(-c4cc(-c5cccc6c5OCC6)c5c(c4)oc4ccccc45)c3)c3ccc(-c4ccccc4)s3)cc2)cc1 ; CC1(C)c2ccccc2-c2c(-c3cccc4c3OCO4)cc(-c3cccc(N(c4ccccc4)c4ccc(-c5ccc6c(c5)sc5ccccc56)n4-c4ccccc4)c3)cc21 ; CC1(C)c2ccccc2-c2cc(-c3cccc4c3SCS4)c(-c3cccc(N(c4ccc(-c5ccccc5)cc4)c4cccc(-c5ccc6c(c5)oc5ccccc56)c4)c3)cc21 ; c1ccc(-c2ccc(N(c3ccc(-c4cc(-c5cccc6c5OCCC6)c5c(c4)sc4ccccc45)cc3)c3cccc(-c4nc5ccccc5o4)c3)cc2)cc1]

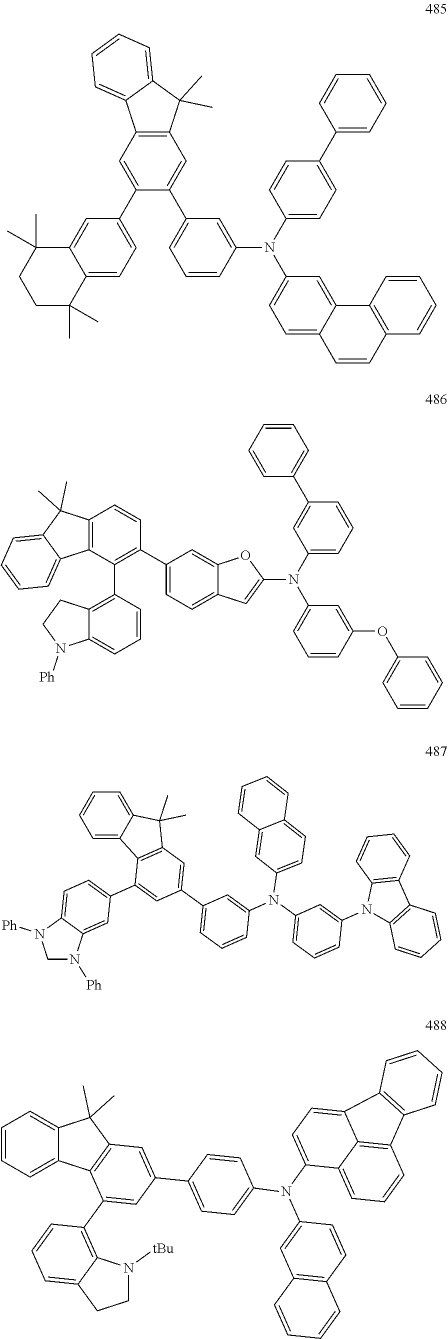 [SMILES: CC1(C)CCC(C)(C)c2cc(-c3cc4c(cc3-c3cccc(N(c5ccc(-c6ccccc6)cc5)c5ccc6ccc7ccccc7c6c5)c3)C(C)(C)c3ccccc3-4)ccc21 ; CC1(C)c2ccccc2-c2c1ccc(-c1ccc3cc(N(c4cccc(Oc5ccccc5)c4)c4cccc(-c5ccccc5)c4)oc3c1)c2-c1cccc2c1CCN2c1ccccc1 ; CC1(C)c2ccccc2-c2c(-c3ccc4c(c3)N(c3ccccc3)CN4c3ccccc3)cc(-c3cccc(N(c4cccc(-n5c6ccccc6c6ccccc65)c4)c4ccc5ccccc5c4)c3)cc21 ; CC1(C)c2ccccc2-c2c(-c3cccc4c3N(C(C)(C)C)CC4)cc(-c3ccc(N(c4ccc5ccccc5c4)c4ccc5c6c(cccc46)-c4ccccc4-5)cc3)cc21]

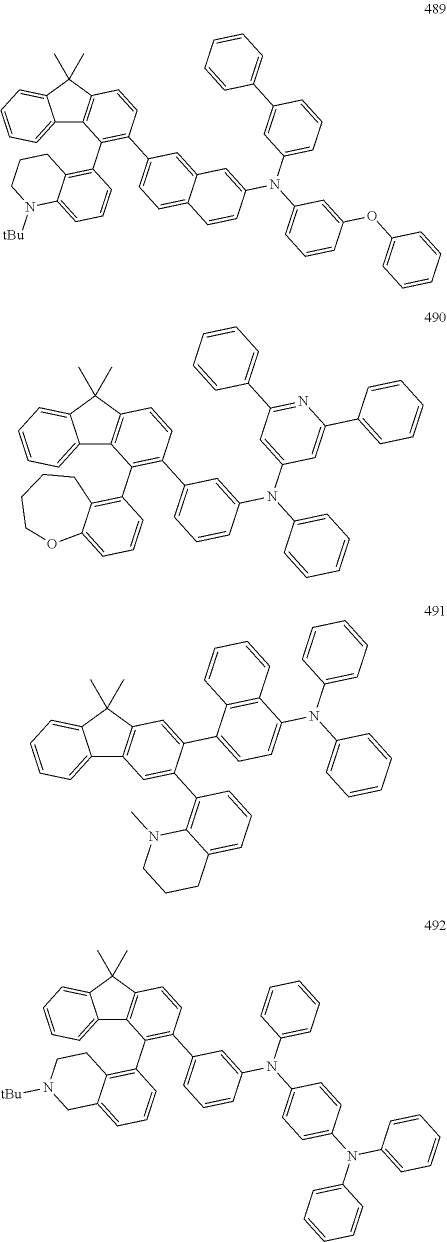 [SMILES: CC1(C)c2ccccc2-c2c1ccc(-c1ccc3ccc(N(c4cccc(Oc5ccccc5)c4)c4cccc(-c5ccccc5)c4)cc3c1)c2-c1cccc2c1CCCN2C(C)(C)C ; CC1(C)c2ccccc2-c2c1ccc(-c1cccc(N(c3ccccc3)c3cc(-c4ccccc4)nc(-c4ccccc4)c3)c1)c2-c1cccc2c1CCCCO2 ; CN1CCCc2cccc(-c3cc4c(cc3-c3ccc(N(c5ccccc5)c5ccccc5)c5ccccc35)C(C)(C)c3ccccc3-4)c21 ; CC1(C)c2ccccc2-c2c1ccc(-c1cccc(N(c3ccccc3)c3ccc(N(c4ccccc4)c4ccccc4)cc3)c1)c2-c1cccc2c1CCN(C(C)(C)C)C2]

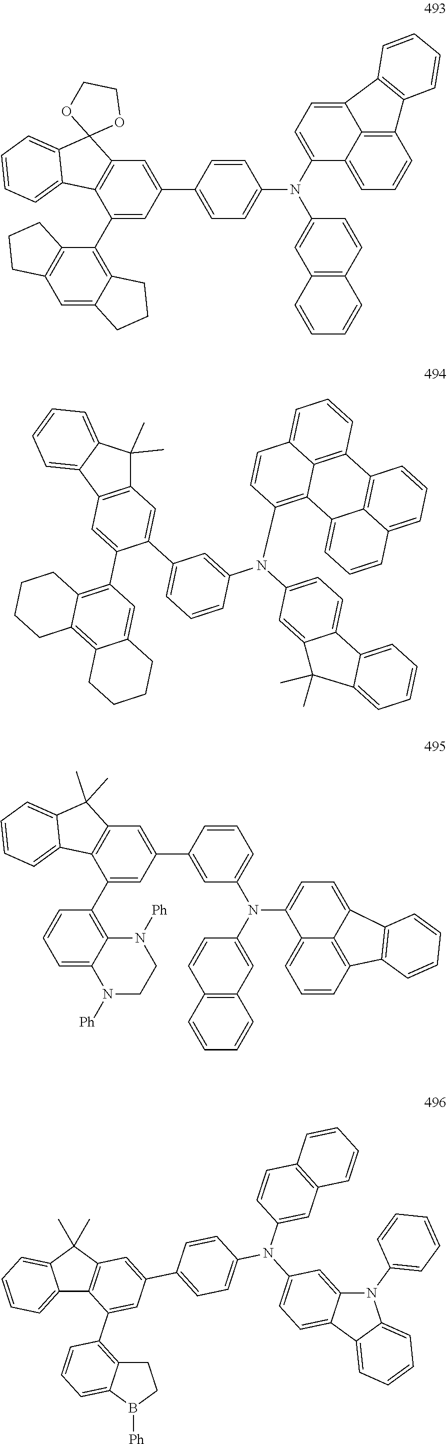 [SMILES: c1ccc2c(c1)-c1cccc3c(N(c4ccc(-c5cc(-c6c7c(cc8c6CCC8)CCC7)c6c(c5)C5(OCCO5)c5ccccc5-6)cc4)c4ccc5ccccc5c4)ccc-2c13 ; CC1(C)c2ccccc2-c2ccc(N(c3cccc(-c4cc5c(cc4-c4cc6c(c7c4CCCC7)CCCC6)-c4ccccc4C5(C)C)c3)c3ccc4cccc5c6cccc7cccc(c3c45)c76)cc21 ; CC1(C)c2ccccc2-c2c(-c3cccc4c3N(c3ccccc3)CCN4c3ccccc3)cc(-c3cccc(N(c4ccc5ccccc5c4)c4ccc5c6c(cccc46)-c4ccccc4-5)c3)cc21 ; CC1(C)c2ccccc2-c2c(-c3cccc4c3CCB4c3ccccc3)cc(-c3ccc(N(c4ccc5ccccc5c4)c4ccc5c6ccccc6n(-c6ccccc6)c5c4)cc3)cc21]

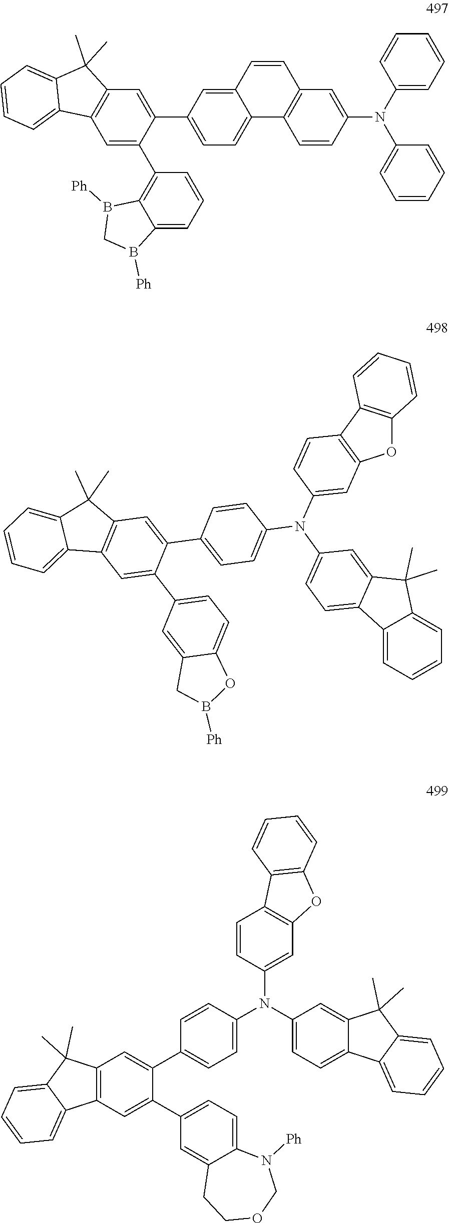 [SMILES: CC1(C)c2ccccc2-c2cc(-c3cccc4c3B(c3ccccc3)CB4c3ccccc3)c(-c3ccc4c(ccc5cc(N(c6ccccc6)c6ccccc6)ccc54)c3)cc21 ; CC1(C)c2ccccc2-c2ccc(N(c3ccc(-c4cc5c(cc4-c4ccc6c(c4)CB(c4ccccc4)O6)-c4ccccc4C5(C)C)cc3)c3ccc4c(c3)oc3ccccc34)cc21 ; CC1(C)c2ccccc2-c2ccc(N(c3ccc(-c4cc5c(cc4-c4ccc6c(c4)CCOCN6c4ccccc4)-c4ccccc4C5(C)C)cc3)c3ccc4c(c3)oc3ccccc34)cc21]

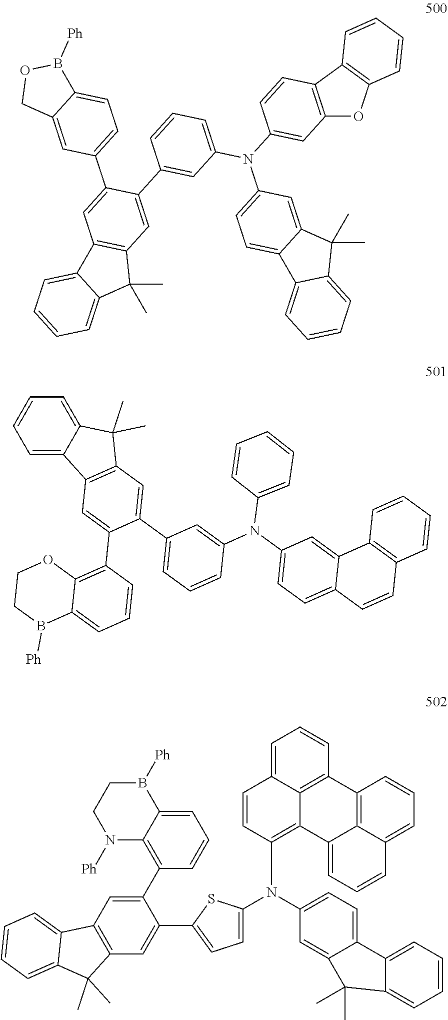 [SMILES: CC1(C)c2ccccc2-c2ccc(N(c3cccc(-c4cc5c(cc4-c4ccc6c(c4)COB6c4ccccc4)-c4ccccc4C5(C)C)c3)c3ccc4c(c3)oc3ccccc34)cc21 ; CC1(C)c2ccccc2-c2cc(-c3cccc4c3OCCB4c3ccccc3)c(-c3cccc(N(c4ccccc4)c4ccc5ccc6ccccc6c5c4)c3)cc21 ; CC1(C)c2ccccc2-c2ccc(N(c3ccc(-c4cc5c(cc4-c4cccc6c4N(c4ccccc4)CCB6c4ccccc4)-c4ccccc4C5(C)C)s3)c3ccc4cccc5c6cccc7cccc(c3c45)c76)cc21]

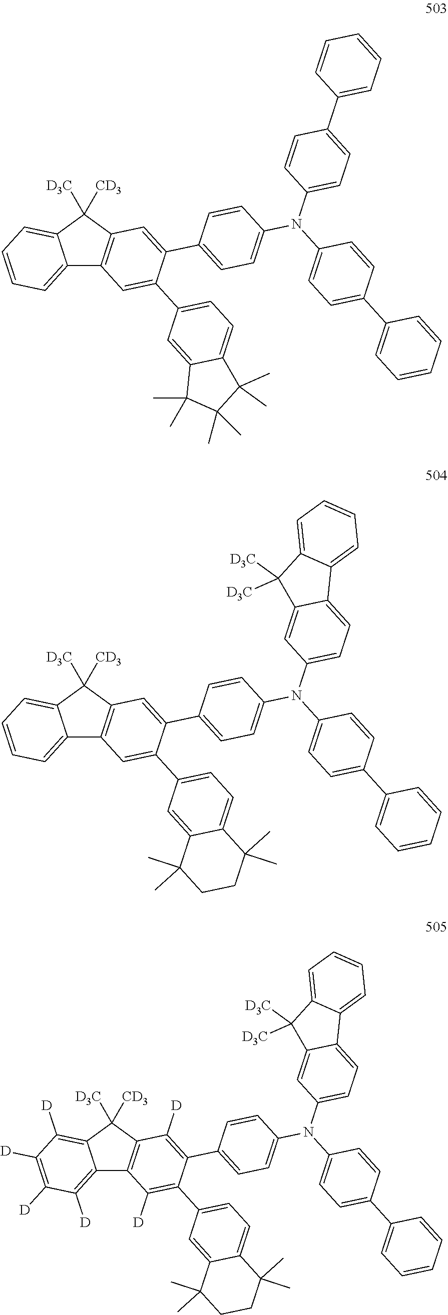

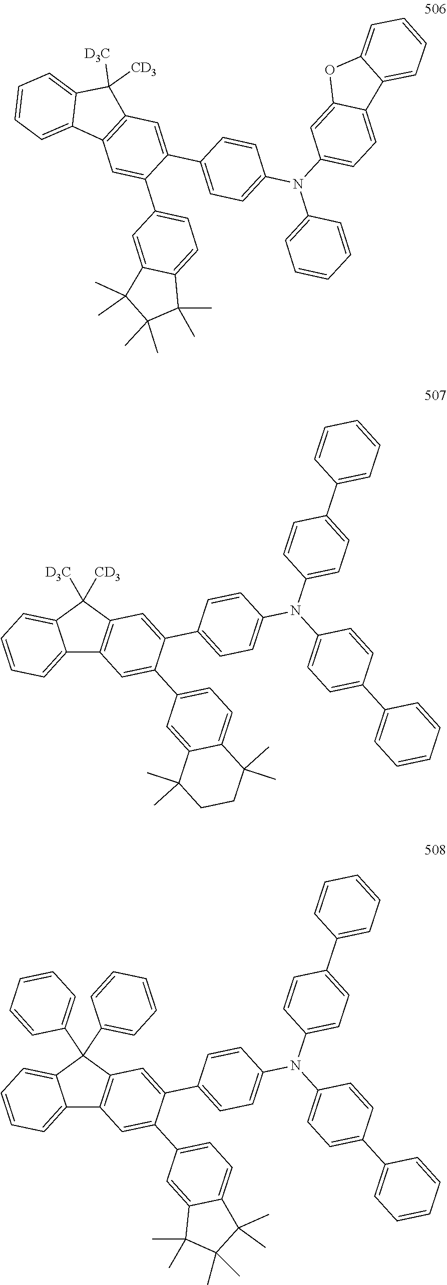 [SMILES: [2H]C([2H])([2H])C1(C([2H])([2H])[2H])c2ccccc2-c2cc(-c3ccc4c(c3)C(C)(C)C(C)(C)C4(C)C)c(-c3ccc(N(c4ccccc4)c4ccc5c(c4)oc4ccccc45)cc3)cc21 ; [2H]C([2H])([2H])C1(C([2H])([2H])[2H])c2ccccc2-c2cc(-c3ccc4c(c3)C(C)(C)CCC4(C)C)c(-c3ccc(N(c4ccc(-c5ccccc5)cc4)c4ccc(-c5ccccc5)cc4)cc3)cc21 ; CC1(C)c2ccc(-c3cc4c(cc3-c3ccc(N(c5ccc(-c6ccccc6)cc5)c5ccc(-c6ccccc6)cc5)cc3)C(c3ccccc3)(c3ccccc3)c3ccccc3-4)cc2C(C)(C)C1(C)C]

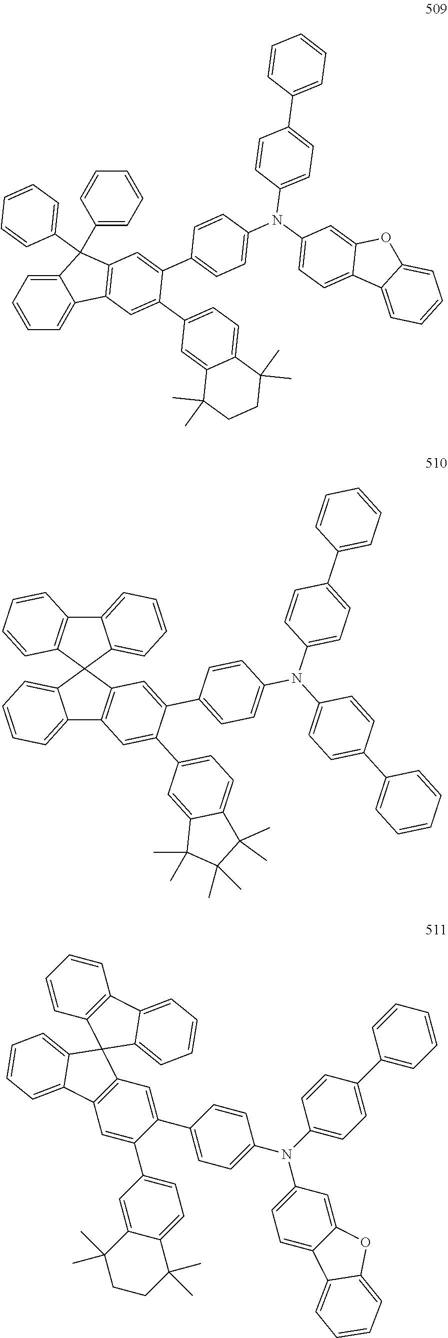 [SMILES: CC1(C)CCC(C)(C)c2cc(-c3cc4c(cc3-c3ccc(N(c5ccc(-c6ccccc6)cc5)c5ccc6c(c5)oc5ccccc56)cc3)C(c3ccccc3)(c3ccccc3)c3ccccc3-4)ccc21 ; CC1(C)c2ccc(-c3cc4c(cc3-c3ccc(N(c5ccc(-c6ccccc6)cc5)c5ccc(-c6ccccc6)cc5)cc3)C3(c5ccccc5-c5ccccc53)c3ccccc3-4)cc2C(C)(C)C1(C)C ; CC1(C)CCC(C)(C)c2cc(-c3cc4c(cc3-c3ccc(N(c5ccc(-c6ccccc6)cc5)c5ccc6c(c5)oc5ccccc56)cc3)C3(c5ccccc5-c5ccccc53)c3ccccc3-4)ccc21]

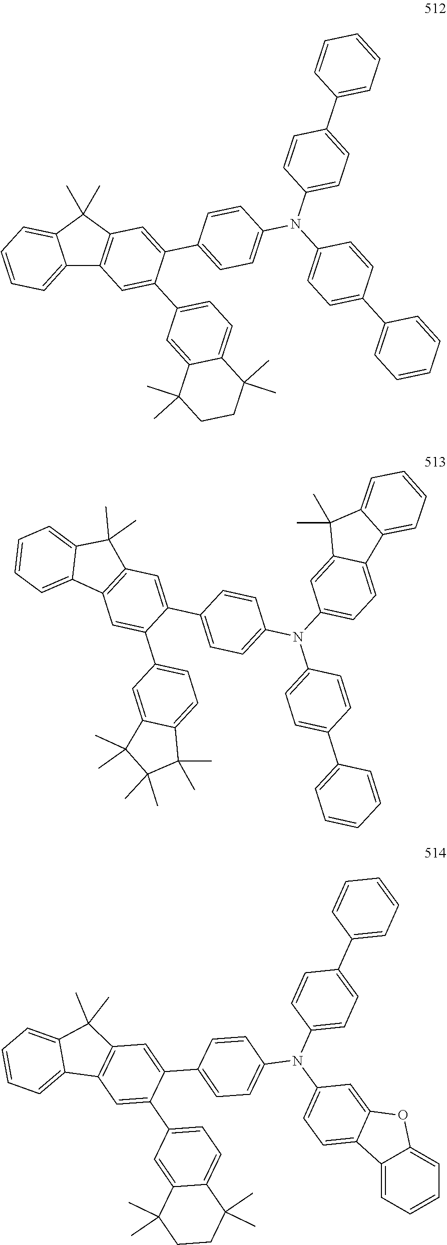 [SMILES: CC1(C)CCC(C)(C)c2cc(-c3cc4c(cc3-c3ccc(N(c5ccc(-c6ccccc6)cc5)c5ccc(-c6ccccc6)cc5)cc3)C(C)(C)c3ccccc3-4)ccc21 ; CC1(C)c2ccccc2-c2ccc(N(c3ccc(-c4ccccc4)cc3)c3ccc(-c4cc5c(cc4-c4ccc6c(c4)C(C)(C)C(C)(C)C6(C)C)-c4ccccc4C5(C)C)cc3)cc21 ; CC1(C)CCC(C)(C)c2cc(-c3cc4c(cc3-c3ccc(N(c5ccc(-c6ccccc6)cc5)c5ccc6c(c5)oc5ccccc56)cc3)C(C)(C)c3ccccc3-4)ccc21]

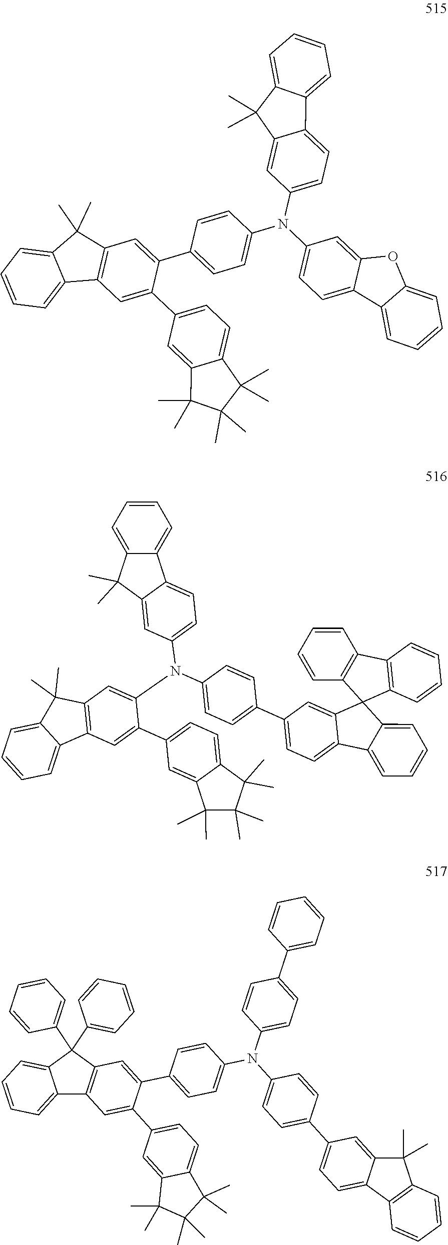 [SMILES: CC1(C)c2ccccc2-c2ccc(N(c3ccc(-c4cc5c(cc4-c4ccc6c(c4)C(C)(C)C(C)(C)C6(C)C)-c4ccccc4C5(C)C)cc3)c3ccc4c(c3)oc3ccccc34)cc21 ; CC1(C)c2ccccc2-c2ccc(N(c3ccc(-c4ccc5c(c4)C4(c6ccccc6-c6ccccc64)c4ccccc4-5)cc3)c3cc4c(cc3-c3ccc5c(c3)C(C)(C)C(C)(C)C5(C)C)-c3ccccc3C4(C)C)cc21 ; CC1(C)c2ccccc2-c2ccc(-c3ccc(N(c4ccc(-c5ccccc5)cc4)c4ccc(-c5cc6c(cc5-c5ccc7c(c5)C(C)(C)C(C)(C)C7(C)C)-c5ccccc5C6(c5ccccc5)c5ccccc5)cc4)cc3)cc21]

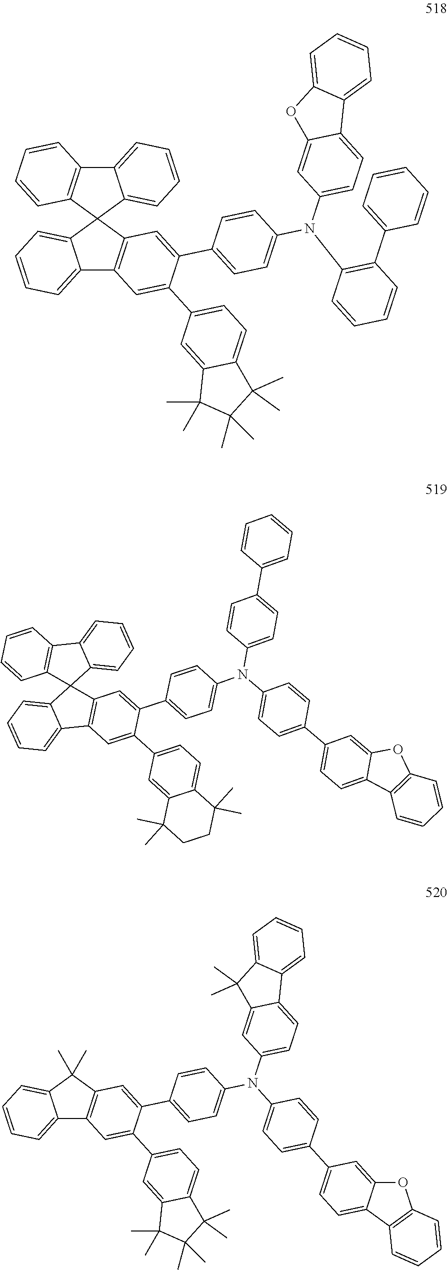 [SMILES: CC1(C)c2ccc(-c3cc4c(cc3-c3ccc(N(c5ccc6c(c5)oc5ccccc56)c5ccccc5-c5ccccc5)cc3)C3(c5ccccc5-c5ccccc53)c3ccccc3-4)cc2C(C)(C)C1(C)C ; CC1(C)CCC(C)(C)c2cc(-c3cc4c(cc3-c3ccc(N(c5ccc(-c6ccccc6)cc5)c5ccc(-c6ccc7c(c6)oc6ccccc67)cc5)cc3)C3(c5ccccc5-c5ccccc53)c3ccccc3-4)ccc21 ; CC1(C)c2ccccc2-c2ccc(N(c3ccc(-c4ccc5c(c4)oc4ccccc45)cc3)c3ccc(-c4cc5c(cc4-c4ccc6c(c4)C(C)(C)C(C)(C)C6(C)C)-c4ccccc4C5(C)C)cc3)cc21]

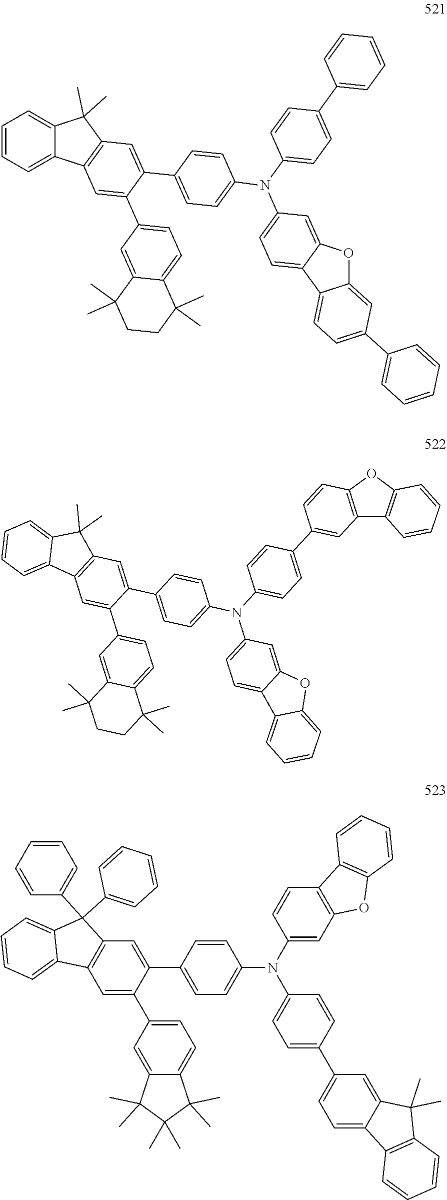 [SMILES: CC1(C)CCC(C)(C)c2cc(-c3cc4c(cc3-c3ccc(N(c5ccc(-c6ccccc6)cc5)c5ccc6c(c5)oc5cc(-c7ccccc7)ccc56)cc3)C(C)(C)c3ccccc3-4)ccc21 ; CC1(C)CCC(C)(C)c2cc(-c3cc4c(cc3-c3ccc(N(c5ccc(-c6ccc7oc8ccccc8c7c6)cc5)c5ccc6c(c5)oc5ccccc56)cc3)C(C)(C)c3ccccc3-4)ccc21 ; CC1(C)c2ccccc2-c2ccc(-c3ccc(N(c4ccc(-c5cc6c(cc5-c5ccc7c(c5)C(C)(C)C(C)(C)C7(C)C)-c5ccccc5C6(c5ccccc5)c5ccccc5)cc4)c4ccc5c(c4)oc4ccccc45)cc3)cc21]

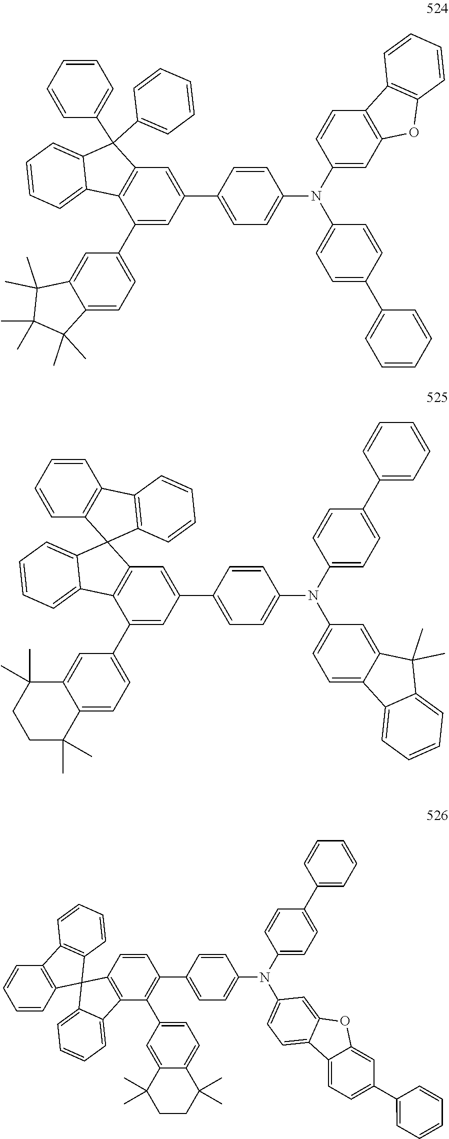

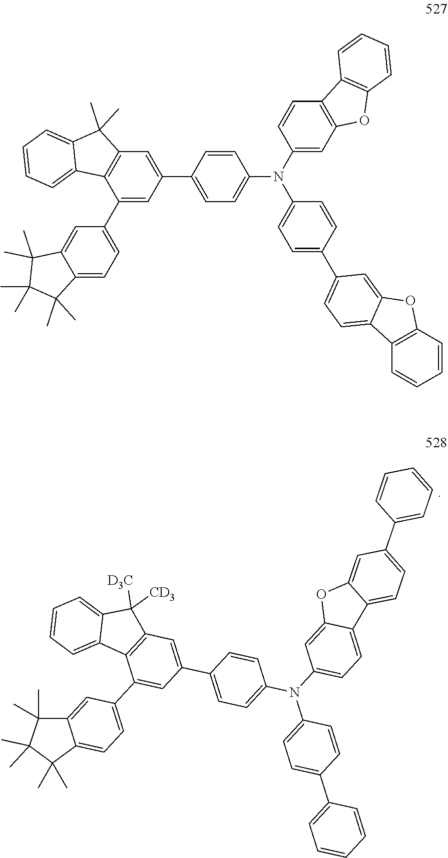 [SMILES: CC1(C)c2ccccc2-c2c(-c3ccc4c(c3)C(C)(C)C(C)(C)C4(C)C)cc(-c3ccc(N(c4ccc(-c5ccc6c(c5)oc5ccccc56)cc4)c4ccc5c(c4)oc4ccccc45)cc3)cc21 ; [2H]C([2H])([2H])C1(C([2H])([2H])[2H])c2ccccc2-c2c(-c3ccc4c(c3)C(C)(C)C(C)(C)C4(C)C)cc(-c3ccc(N(c4ccc(-c5ccccc5)cc4)c4ccc5c(c4)oc4cc(-c6ccccc6)ccc45)cc3)cc21]

20. An organic optoelectronic device, comprising a first electrode, a second electrode and an organic layer, wherein the organic layer is at least one of a hole injection layer, a hole transport layer, a light-emitting layer, an electron injection layer or an electron transport layer, and the organic layer comprising a compound having a chemical structure shown in formula (I): 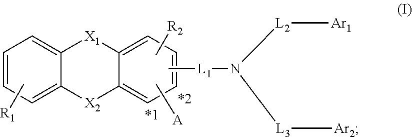
where group A is one or more selected from of the following groups: 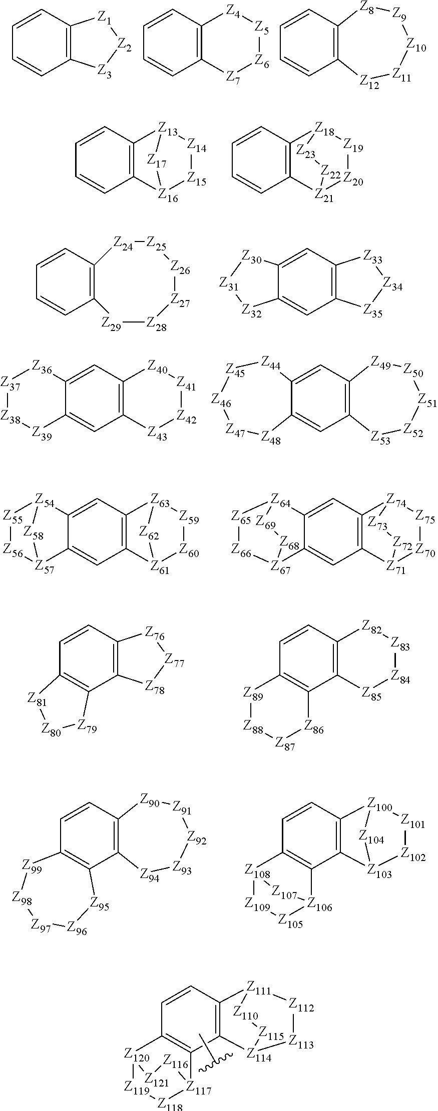
Z₁-Z₇₅, Z₇₆-Z₁₂1 are each independently selected from CR₃R₄, NR, SiR₆R₇, BRs, O or S; *1, *2 are connection sites of group A; *1 or *2 is capable of being connected to any position on group A;
X₁, X₂ are each independently selected from a single bond, CR₉R₁₀, NR₁₁, SiR₁₂R₁₃, 0 or S; or R₉, R₁₀ are bonded to form a ring;
R₁-R₁₃ are the same or different, and are each independently selected from hydrogen, deuterium, substituted or unsubstituted straight or branched C1-C30 alkyl groups, substituted or unsubstituted C1-C30 heteroalkyl groups, substituted or unsubstituted C3-C30 cycloalkyl groups, substituted or unsubstituted C3-C30 heterocycloalkyl groups, substituted or unsubstituted C6-C30 aryl groups, or substituted or unsubstituted C6-C30 heteroaryl groups;
L₁-L₃ are the same or different, and are each independently selected from a single bond, substituted or unsubstituted C6-C30 arylene groups, or substituted or unsubstituted C3-C30 heteroarylene groups;
A₁ and Ar₂ are the same or different, and are each independently selected from substituted or unsubstituted C6-C30 aryl groups, or substituted or unsubstituted C6-C30 heteroaryl groups.
